(19) United States
(12) Patent Application Publication
(10) Pub. No.: US 2020/0392470 A1
(43) Pub. Date: Dec. 17, 2020

(54) NOVEL ACYLTRANSFERASES, VARIANT THIOESTERASES, AND USES THEREOF

(71) Applicant: Corbion Biotech, Inc., South San Francisco, CA (US)

(72) Inventors: Jeffrey Leo Moseley, Redwood City, CA (US); Jason Casolari, Palo Alto, CA (US); Xinhua Zhao, Dublin, CA (US); Aren Ewing, South San Francisco, CA (US); Aravind Somanchi, Redwood City, CA (US); Scott Franklin, La Jolla, CA (US); David Davis, South San Francisco, CA (US)

(21) Appl. No.: 16/998,268

(22) Filed: Aug. 20, 2020

Related U.S. Application Data
(63) Continuation of application No. 15/725,222, filed on Oct. 4, 2017, now abandoned.
(60) Provisional application No. 62/404,667, filed on Oct. 5, 2016.

(51) Int. Cl. C12N 9/10; C12N 15/82; C12P 7/64

(52) U.S. Cl. CPC C12N 9/1029; C12N 9/1025; C12N 2800/22; C12P 7/6463; C12Y 203/01051; C12N 15/82

(57) ABSTRACT

Disclosed are microalgal cells having an ablated or downregulated fatty acyl-ACP thioesterase (FATA) gene, wherein the cell is modified to express a heterologous lysophosphatidic acid acyltransferase (LPAAT) comprising an amino acid sequence that has at least 80% identity to an acyltransferase encoded by SEQ ID NO: 90, 89, 92, 93 or 95 and wherein the modified microalgal cell produces an oil with an elevated ratio of saturated-unsaturated-saturated triglycerides over trisaturated triglycerides as compared to a corresponding unmodified cell. Also disclosed are microalgal oils comprising at least 60% stearate-oleate-stearate (SOS) triglycerides, less than 5% trisaturates and wherein the fatty acid profile of the oil comprises at least 50% C18:0. Related methods of producing an oil are also disclosed.

### CROSS-REFERENCE TO RELATED APPLICATIONS

This application is a continuation of U.S. application Ser. No. 15/725,222, filed Oct. 4, 2017, which claims the benefit under 35 U.S.C. 119(e) of U.S. Provisional Patent Application No. 62/404,667, filed Oct. 5, 2016, entitled “Novel Acyltransferases, Variant Thioesterases, And Uses Thereof”, each of which is incorporated herein by reference in its entirety for all purposes.

### INCORPORATION-BY-REFERENCE OF MATERIAL SUBMITTED ELECTRONICALLY

This application includes a list of sequences, as shown at the end of the detailed description. The instant application contains a Sequence Listing which has been submitted electronically in ASCII format and is hereby incorporated by reference in its entirety. Said ASCII copy, dated Aug. 11, 2020, is named CORBP072US_SL.txt and is 606,605 bytes in size.

### FIELD OF THE INVENTION

Embodiments of the present invention relate to oils/fats, fuels, foods, and oleochemicals and their production from cultures of genetically engineered cells. Embodiments relate to nucleic acids and proteins that are involved in the fatty acid synthetic pathways; oils with a high content of triglycerides bearing fatty acyl groups upon the glycerol backbone in particular regiospecific patterns, highly stable oils, oils with high levels of oleic or mid-chain fatty acids, and products produced from such oils.

### BACKGROUND OF THE INVENTION

Co-owned patent applications WO2008/151149, WO2010/063031, WO2010/063032, WO2011/150410, WO2011/150411, WO2012/061647, WO2012/061647, WO2012/106560, WO2013/158938, WO2014/120829, WO2014/151904, WO2015/051319, WO2016/007862, WO2016/014968, WO2016/044779, and WO2016/164495 relate to microbial oils and methods for producing those oils in host cells, including microalgae. These publications also describe the use of such oils to make foods, oleochemicals, fuels and other products.

Certain enzymes of the fatty acyl-CoA elongation pathway function to extend the length of fatty acyl-CoA molecules. Elongase-complex enzymes extend fatty acyl-CoA molecules in 2 carbon additions, for example myristoyl-CoA to palmitoyl-CoA, stearoyl-CoA to arachidyl-CoA, or oleoyl-CoA to eicosanoyl-CoA, eicosanoyl-CoA to erucyl-CoA. In addition, elongase enzymes also extend acyl chain length in 2 carbon increments. KCS enzymes condense acyl-CoA molecules with two carbons from malonyl-CoA to form beta-ketoacyl-CoA. KCS and elongases may show specificity for condensing acyl substrates of particular carbon length, modification (such as hydroxylation), or degree of saturation. For example, the jojoba (Simmondsia chinensis) beta-ketoacyl-CoA synthase has been demonstrated to prefer monounsaturated and saturated C18- and C20-CoA substrates to elevate production of erucic acid in transgenic plants (Lassner et al., Plant Cell, 1996, Vol 8(2), pp. 281-292), whereas specific elongase enzymes of Trypanosoma brucei show preference for elongating short and midchain saturated CoA substrates (Lee et al., Cell, 2006, Vol 126(4), pp. 691-9).

The type II fatty acid biosynthetic pathway employs a series of reactions catalyzed by soluble proteins with intermediates shuttled between enzymes as thioesters of acyl carrier protein (ACP). By contrast, the type I fatty acid biosynthetic pathway uses a single, large multifunctional polypeptide.

The oleaginous, non-photosynthetic alga, Prototheca moriformis, stores copious amounts of triacylglyceride oil under conditions when the nutritional carbon supply is in excess, but cell division is inhibited due to limitation of other essential nutrients. Bulk biosynthesis of fatty acids with carbon chain lengths up to C18 occurs in the plastids; fatty acids are then exported to the endoplasmic reticulum where (if it occurs) elongation past C18 and incorporation into triacylglycerides (TAGs) is believed to occur. Lipids are stored in large cytoplasmic organelles called lipid bodies until environmental conditions change to favor growth, whereupon they are mobilized to provide energy and carbon molecules for anabolic metabolism.

### SUMMARY OF THE INVENTION

In various aspects, the inventions disclosed herein include one or more of the following embodiments. The embodiments can be practiced alone or in combination with each other.

### Embodiment 1

This embodiment of the invention provides a recombinant vector construct or a host cell comprising nucleic acids that encode an acyltransferase that optionally is operable to produce an altered fatty acid profile or an altered sn-2 profile in an oil produced by a host cell expressing the nucleic acids. The nucleic acids can be a nucleic acid construct or a vector construct that also includes one or more regulatory elements. The one or more regulatory elements include promoters, targeting sequences, secretion signals and other elements that control or direct the expression of the encoded protein in the host cell. The acyltransferase encoded by the nucleic acids have 75%, 80%, 85%, 90%, 95%, 98%, 99%, or 100%, or at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196. The acyl transferases of this invention is a lysophosphatidic acid acyltransferase (LPAAT), glycerol phosphate acyltransferase (GPAT), diacyl glycerol acyltransferase (DGAT), lysophosphatidylcholine acyltransferase (LPCAT), or phospholipase A2 (PLA2). The acyltransferases of the invention are shown in Table 5. In one embodiment, the acyltransferases of the invention have acyltransferase activity and the amino acid sequence comprises at least 96.3%, 98%, or 99% identity to an acyltransferase of clade 1 of Table 5. In another embodiment, the acyltransferases of the invention have acyltransferase activity and the amino acid sequence comprises at least 93.9%, 98%, or 99% identity to an acyltransferase of clade 2 of Table 5. In one embodiment, the acyltransferases of the invention have acyltransferase activity and the amino acid sequence comprises at least 86.5%, 90%, 95%, 98%, or 99% identity to an acyltransferase of clade 3 of Table 5. In one embodiment, the acyltransferases of the invention have acyltransferase activity and the amino acid sequence comprises at least 78.5%, 80%, 85%, 90%, 95%, 98%, or 99% identity to an acyltransferase of clade 4 of Table 5. In one embodiment, the recombinant vector construct of host cell comprises nucleic acids that 75%, 80%, 85%, 90%, 95%, 98%, 99%, or 100%, or at least 75%, 80%, 85%, 90%, 95%, 98%, 99%, or 100% identity to an acyltransferase encoded by SEQ ID NOs: 19, 20, 21, 22, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125.

### Embodiment 2

This embodiment of the invention provides nucleic acids that encode an acyltransferase that when expressed produces an altered fatty acid profile or an altered sn-2 profile in an oil produced by a host cell expressing the nucleic acids. The nucleic acids can be a nucleic acid construct or a vector construct that also includes one or more regulatory elements. The one or more regulatory elements include promoters, targeting sequences, secretion signals and other elements that control or direct the expression of the encoded protein in the host cell. The acyltransferase encoded by the nucleic acids have 75%, 80%, 85%, 90%, 95%, 98%, 99%, or 100%, or at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196. The acyl transferases of this invention is a lysophosphatidic acid acyltransferase (LPAAT), glycerol phosphate acyltransferase (GPAT), diacyl glycerol acyltransferase (DGAT), lysophosphatidylcholine acyltransferase (LPCAT), or phospholipase A2 (PLA2). The acyltransferases of the invention are shown in Table 5. In one embodiment, the acyltransferases of the invention have acyltransferase activity and the amino acid sequence comprises at least 96.3%, 98%, or 99% identity to an acyltransferase of clade 1 of Table 5. In another embodiment, the acyltransferases of the invention have acyltransferase activity and the amino acid sequence comprises at least 93.9%, 98%, or 99% identity to an acyltransferase of clade 2 of Table 5. In one embodiment, the acyltransferases of the invention have acyltransferase activity and the amino acid sequence comprises at least 86.5%, 90%, 95%, 98%, or 99% identity to an acyltransferase of clade 3 of Table 5. In one embodiment, the acyltransferases of the invention have acyltransferase activity and the amino acid sequence comprises at least 78.5%, 80%, 85%, 90%, 95%, 98%, or 99% identity to an acyltransferase of clade 4 of Table 5. In one embodiment, the nucleic acids comprise nucleic acids that are 75%, 80%, 85%, 90%, 95%, 98%, 99%, or 100%, or at least 75%, 80%, 85%, 90%, 95%, 98%, 99%, or 100% identity to an acyltransferase encoded by SEQ ID NOs: 19, 20, 21, 22, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125.

### Embodiment 3

This embodiment of the invention provides codon-optimized nucleic acids that encodes an acyltransferase operable to produce an altered fatty acid profile and/or an altered sn-2 profile in an oil produced by a host cell expressing the nucleic acids. In one aspect, the codons are optimized for expression in the host cell, including host cells derived from plants. In another aspect, the codons are optimized for expression in Prototheca or Chlorella. In a further aspect the codons are optimized for expression in Prototheca moriformis or Chlorella protothecoides. The codon-optimized nucleic acids can be a nucleic acid construct or a vector construct that also includes one or more regulatory elements. The one or more regulatory elements are also codon-optimized for Prototheca or Chlorella. The one or more regulatory elements include promoters, targeting sequences, secretion signals and other elements that control or direct the expression of the encoded protein in the host cell. The acyltransferase encoded by the codon-optimized nucleic acids have 75%, 80%, 85%, 90%, 95%, 98%, 99%, or 100%, or at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196. When the codons are optimized for expression in a host organism, at least 60%, 65%, 70%, 75%, 80%, 85%, 90%, 95%, or 100% of the codons used is the most preferred codon. Alternately, at least 60%, 65%, 70%, 75%, 80%, 85%, 90%, 95%, or 100% of the codons used is the first or second most preferred codon. The codon-optimized nucleic acids encode acyltransferases that are shown in Table 5. In one embodiment, the acyltransferases of the invention have acyltransferase activity and the amino acid sequence comprises at least 96.3%, 98%, or 99% identity to an acyltransferase of clade 1 of Table 5. In another embodiment, the acyltransferases of the invention have acyltransferase activity and the amino acid sequence comprises at least 93.9%, 98%, or 99% identity to an acyltransferase of clade 2 of Table 5. In one embodiment, the acyltransferases of the invention have acyltransferase activity and the amino acid sequence comprises at least 86.5%, 90%, 95%, 98%, or 99% identity to an acyltransferase of clade 3 of Table 5. In one embodiment, the acyltransferases of the invention have acyltransferase activity and the amino acid sequence comprises at least 78.5%, 80%, 85%, 90%, 95%, 98%, or 99% identity to an acyltransferase of clade 4 of Table 5. The acyltransferase encoded by the codon-optimized nucleic acids have 75%, 80%, 85%, 90%, 95%, 98%, 99%, or 100%, or at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196. In one embodiment, the codon-optimizes nucleic acids comprise nucleic acids that 75%, 80%, 85%, 90%, 95%, 98%, 99%, or 100%, or at least 75%, 80%, 85%, 90%, 95%, 98%, 99%, or 100% identity to an acyltransferase encoded by SEQ ID NOs: 19, 20, 21, 22, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125.

### Embodiment 4

In this embodiment, the invention provides host cells that are oleaginous microorganism cells or plant cells. The microorganisms of the invention are eukaryotic microorganism. In one aspect, the host cells are microalgae. In one embodiment, the microalgae are of the phylum Chlorophyta, the class Trebouxiophytae, the order Chlorellales, or the family Chlorellacae. In one embodiment, the microalgae are of the genus Prototheca or Chlorella. In one embodiment, the microalgae are of the species Prototheca moriformis, Prototheca zopfii, Prototheca wickerhamii Prototheca blaschkeae, Prototheca chlorelloides, Prototheca crieana, Prototheca dilamenta, Prototheca hydrocarbonea, Prototheca kruegeri, Prototheca portoricensis, Prototheca salmonis, Prototheca segbwema, Prototheca stagnorum, Prototheca trispora Prototheca ulmea, or Prototheca viscosa. Preferably, the microalga is of the species Prototheca moriformis. In one embodiment, the microalgae are of the species Chlorella autotrophica, Chlorella colonials, Chlorella lewinii, Chlorella minutissima, Chlorella pituitam, Chlorella pulchelloides, Chlorella pyrenoidosa, Chlorella rotunda, Chlorella singularis, Chlorella sorokiniana, Chlorella variabilis, or Chlorella volutis. Preferably, the microalga is of the species Chlorella protothecoides or Auxenochlorella protothecoides. The host cells express the nucleic acids for Embodiments relating to acyltransferases of the invention.

### Embodiment 5

In this embodiment, the acyl transferase is lysophosphatidic acid acyltransferase (LPAAT), glycerol phosphate acyltransferase (GPAT), diacyl glycerol acyltransferase (DGAT), lysophosphatidylcholine acyltransferase (LPCAT), or phospholipase A2 (PLA2). In one embodiment, the acyltransferases of the invention are shown in Table 5. In one embodiment, the acyltransferases of the invention have acyltransferase activity and the amino acid sequence comprises at least 96.3%, 98%, or 99% identity to an acyltransferase of clade 1 of Table 5. In another embodiment, the acyltransferases of the invention have acyltransferase activity and the amino acid sequence comprises at least 93.9%, 98%, or 99% identity to an acyltransferase of clade 2 of Table 5. In one embodiment, the acyltransferases of the invention have acyltransferase activity and the amino acid sequence comprises at least 86.5%, 90%, 95%, 98%, or 99% identity to an acyltransferase of clade 3 of Table 5. In one embodiment, the acyltransferases of the invention have acyltransferase activity and the amino acid sequence comprises at least 78.5%, 80%, 85%, 90%, 95%, 98%, or 99% identity to an acyltransferase of clade 4 of Table 5. The acyltransferase have 75%, 80%, 85%, 90%, 95%, 98%, 99%, or 100%, or at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196.

### Embodiment 6

In this embodiment, nucleic acids encoding acyltransferases increases the production of C8:0 and/or C10:0 fatty acids or alters the sn-2 profile in the host cell. The acyltransferases of the invention have acyltransferase activity and the amino acid sequence comprises at least 96.3%, 98%, or 99% identity to an acyltransferase of clade 1 of Table 5. In another embodiment, the acyltransferases of the invention have acyltransferase activity and the amino acid sequence comprises at least 93.9%, 98%, or 99% identity to an acyltransferase of clade 2 of Table 5. In one embodiment, the acyltransferases of the invention have acyltransferase activity and the amino acid sequence comprises at least 86.5%, 90%, 95%, 98%, or 99% identity to an acyltransferase of clade 3 of Table 5. In one embodiment, the acyltransferases of the invention have acyltransferase activity and the amino acid sequence comprises at least 78.5%, 80%, 85%, 90%, 95%, 98%, or 99% identity to an acyltransferase of clade 4 of Table 5. The C8:0 or the C10:0 content of the oil of the host cell is increased by 5%, 10%, 15%, 20%, 25%, 30%, 35%, 40%, 45%, 50%, 55%, 60%, 70%, 80%, 90%, or higher as compared the C8:0 and/or C10:0 content of a cell oil that does not express the recombinant nucleic acids encoding the LPAATs of the invention. The sn-2 profile of the oil is altered by the expression of the LPAATs of the invention and/or the C8:0 and/or C10:0 fatty acid at the sn-2 position is increased by 5%, 10%, 15%, 20%, 25%, 30%, 35%, 40%, 45%, 50%, 55%, 60%, 70%, 80%, 90%, or higher as compared to the C8:0 and/or C10:0 fatty acid at the sn-2 position of the cell oil that does not express the recombinant nucleic acids encoding the LPAATs of the invention. The acyltransferase encoded by the codon-optimized nucleic acids have 75%, 80%, 85%, 90%, 95%, 98%, 99%, or 100%, or at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196.

### Embodiment 7

This embodiment comprises nucleic acids encoding LPAATs, shown in Table 5, and disclosed herein. The LPAATs encoded by the nucleic acids have 75%, 80%, 85%, 90%, 95%, 98%, 99%, or 100%, or at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, or 180.

### Embodiment 8

In this embodiment, nucleic acids encoding GPATs of the invention have 75%, 80%, 85%, 90%, 95%, 98%, 99%, or 100%, or at least 75%, 80%, 85%, 90%, 95%, 98%, 99%, or 100% identity to SEQ ID NOs: 181, 182, 183, 184, 185, or 186.

### Embodiment 9

In this embodiment, nucleic acids encoding DGATs of the invention have 75%, 80%, 85%, 90%, 95%, 98%, 99%, or 100%, or at least 75%, 80%, 85%, 90%, 95%, 98%, 99%, or 100% identity to SEQ ID NOs: 187, or 188.

### Embodiment 10

In this embodiment, nucleic acids encoding LPCATs of the invention have 75%, 80%, 85%, 90%, 95%, 98%, 99%, or 100%, or at least 75%, 80%, 85%, 90%, 95%, 98%, 99%, or 100% identity to SEQ ID NOs: 189, 190, 191, or 192,

### Embodiment 11

This embodiment comprises nucleic acids encoding PLA2s. The PLA2s encoded by the nucleic acids have 75%, 80%, 85%, 90%, 95%, 98%, 99%, or 100%, or at least 75%, 80%, 85%, 90%, 95%, 98%, 99%, or 100% identity to SEQ ID NOs: 193, 194, 195, or 196.

### Embodiment 12

This embodiment is a method of cultivating a host cell expressing nucleic acids that encode the one or more acyl transferases of embodiments 1-11

### Embodiment 13

This embodiment is a method of producing an oil by cultivating host cells that express nucleic acids that encode the one or more acyl transferases of Embodiments 1-12 and recovering the oil.

### Embodiment 14

This embodiment is an oil produced by cultivating host cells that express the one or more nucleic acids that encode the acyltransferases of Examples 1-11, and recovering the oil from the host cell. When the host cell is a microalgae, the cell oil produced by the host cell has sterols that are different than the sterols produced by a plant cell. The cell oil has a sterol profile that is different than an oil obtained from a plant.

### Embodiment 15

In this embodiment, a recombinant acyltransferase is provided. The recombinant acyltransferase can be produced by a host cell. The glycosylation of the recombinant acyl transferase is altered from the glycosylation pattern observed in the acyl transferase produced by the non-recombinant, wild-type cell from which the gene encoding the acyl transferase was derived. In one embodiment, the recombinant acyltransferase the invention have acyltransferase activity and the amino acid sequence comprises at least 96.3%, 98%, or 99% identity to an acyltransferase of clade 1 of Table 5. In one embodiment, the recombinant acyltransferase the invention have acyltransferase activity and the amino acid sequence comprises at least 93.9%, 98%, or 99% identity to an acyltransferase of clade 2 of Table 5. In one embodiment, the acyltransferases of the invention have acyltransferase activity and the amino acid sequence comprises at least 86.5%, 90%, 95%, 98%, or 99% identity to an acyltransferase of clade 3 of Table 5. In one embodiment, the acyltransferases of the invention have acyltransferase activity and the amino acid sequence comprises at least 78.5%, 80%, 85%, 90%, 95%, 98%, or 99% identity to an acyltransferase of clade 4 of Table 5. The acyltransferase encoded have 75%, 80%, 85%, 90%, 95%, 98%, 99%, or 100%, or at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196.

### Embodiment 16

This embodiment of the invention provides a recombinant vector construct or a host cell comprising nucleic acids that encode a variant Brassica fatty acyl-ACP thioesterase that optionally is operable to produce an altered fatty acid profile in an oil produced by a host cell expressing the nucleic acids. The nucleic acids can be a nucleic acid construct or a vector construct that also includes one or more regulatory elements. The one or more regulatory elements include promoters, targeting sequences, secretion signals and other elements that control or direct the expression of the encoded protein in the host cell. The thioesterase encoded by the nucleic acids have 75%, 80%, 85%, 90%, 95%, 98%, 99%, or 100%, or at least 75%, 80%, 85%, 90%, 95%, 98%, 99%, or 100% identity to SEQ ID NOs: 165, 166, 167, or 168 and comprise one or more of amino acid variants D124A, D209A, D127A or D212A. In one embodiment, the Brassica RAPA, Brassica napus or the Brassica juncea thioesterases of the invention have fatty acyl hydrolysis activity and prefer to hydrolyze long chain fatty acyl groups from the acyl carrier protein. In one embodiment, the thioesterase genes, isolated from higher plants, are altered to create variant thioesterases that have certain amino acids that have been altered from the wild type enzyme. Due to the altered amino acid(s), the substrate specificity of the thioesterase is altered. The variant BnOTE enzymes increased C18:0 content by DCW, decreased C18:1 content by DCW, and decreased C18:2 content by DCW in host cells and the oils recovered from the host cells.

### Embodiment 17

This embodiment of the invention provides a recombinant vector construct or a host cell comprising nucleic acids that encode a Garcinia mangostana variant fatty acyl-ACP thioesterase (GmFATA) that optionally is operable to produce an altered fatty acid profile in an oil produced by a host cell expressing the nucleic acids. The nucleic acids can be a nucleic acid construct or a vector construct that also includes one or more regulatory elements. The one or more regulatory elements include promoters, targeting sequences, secretion signals and other elements that control or direct the expression of the encoded protein in the host cell. The variant Garcinia thioesterase encoded by the nucleic acids have 75%, 80%, 85%, 90%, 95%, 98%, 99%, or 100%, or at least 75%, 80%, 85%, 90%, 95%, 98%, 99%, or 100% identity to SEQ ID NOs: 137, 138, 139, 140, 141, 142, 143, 144, 145, 146, 147, 148, 149, 150, comprise one more of amino acid variants D variants L91F, L91K, L91S, G96A, G96T, G96V, G108A, G108V, S111A, S111V T156F, T156A, T156K, T156V, or V193A. In one embodiment, the G. mangostana thioesterases of the invention have fatty acyl hydrolysis activity and prefer to hydrolyze long chain fatty acyl groups from the acyl carrier protein. In one embodiment, the thioesterase genes, isolated from higher plants, are altered to create variant thioesterases that have certain amino acids that have been altered from the wild type enzyme. Due to the altered amino acid(s), the substrate specificity of the thioesterase is altered. The variant BnOTE enzymes increased C18:0 content by DCW, decreased C18:1 content by DCW, and decreased C18:2 content by DCW in host cells and the oils recovered from the host cells.

### Embodiment 18

This embodiment of the invention provides nucleic acids that encode variant Brassica thioesterases or variant Garcinia thioestrases that when expressed produce an altered fatty acid profile in an oil produced by a host cell expressing the nucleic acids. The nucleic acids can be a nucleic acid construct or a vector construct that also includes one or more regulatory elements. The one or more regulatory elements include promoters, targeting sequences, secretion signals and other elements that control or direct the expression of the encoded protein in the host cell. The variant Brassica thioesterases encoded by the nucleic acids have 75%, 80%, 85%, 90%, 95%, 98%, 99%, or 100%, or at least 75%, 80%, 85%, 90%, 95%, 98%, 99%, or 100% identity to SEQ ID NOs: 165, 166, 167, or 168 and comprise one or more of amino acid variants D124A, D209A, D127A or D212A. The variant variant Garcinia thioestrases encoded by the nucleic acids have 75%, 80%, 85%, 90%, 95%, 98%, 99%, or 100%, or at least 75%, 80%, 85%, 90%, 95%, 98%, 99%, or 100% identity to SEQ ID NOs: 137, 138, 139, 140, 141, 142, 143, 144, 145, 146, 147, 148, 149, 150 and comprise one or more of amino acid variants L91F, L91K, L91S, G96A, G96T, G96V, G108A, G108V, S111A, S111V T156F, T156A, T156K, T156V, or V193A.

### Embodiment 19

This embodiment of the invention provides codon-optimized nucleic acids that encodes a variant Brassica thioesterase or a variant Garcinia thioestrase operable to produce an altered fatty acid profile in an oil produced by a host cell expressing the nucleic acids. In one aspect, the codons are optimized for expression in the host cell, including host cells derived from plants. In another aspect, the codons are optimized for expression in Prototheca or Chlorella. In a further aspect the codons are optimized for expression in Prototheca moriformis or Chlorella protothecoides. The codon-optimized nucleic acids can be a nucleic acid construct or a vector construct that also includes one or more regulatory elements. The one or more regulatory elements are also codon-optimized for Prototheca or Chlorella. The one or more regulatory elements include promoters, targeting sequences, secretion signals and other elements that control or direct the expression of the encoded protein in the host cell. The variant Brassica thioesterases encoded by the nucleic acids have 75%, 80%, 85%, 90%, 95%, 98%, 99%, or 100%, or at least 75%, 80%, 85%, 90%, 95%, 98%, 99%, or 100% identity to SEQ ID NOs: 165, 166, 167, or 168 and comprise one or more of amino acid variants D124A, D209A, D127A or D212A. The variant variant Garcinia thioestrases encoded by the nucleic acids have 75%, 80%, 85%, 90%, 95%, 98%, 99%, or 100%, or at least 75%, 80%, 85%, 90%, 95%, 98%, 99%, or 100% identity to SEQ ID NOs: 137, 138, 139, 140, 141, 142, 143, 144, 145, 146, 147, 148, 149, or 150 and comprise one or more of amino acid variants L91F, L91K, L91S, G96A, G96T, G96V, G108A, G108V, S111A, S111V T156F, T156A, T156K, T156V, or V193A. When the codons are optimized for expression in a host organism, at least 60%, 65%, 70%, 75%, 80%, 85%, 90%, 95%, or 100% of the codons used is the most preferred codon. Alternately, at least 60%, 65%, 70%, 75%, 80%, 85%, 90%, 95%, or 100% of the codons used is the first or second most preferred codon. The codon-optimized nucleic acids encode variant Brassica thioesterases and variant Garcinia thioestrases. In one embodiment, the variant Brassica thioesterases and variant Garcinia thioestrases of the invention have thioesterase activity.

### Embodiment 20

In this embodiment, the invention provides host cells that are oleaginous microorganism cells or plant cells. The microorganisms of the invention are eukaryotic microorganism. In one aspect, the host cells are microalgae. In one embodiment, the microalgae are of the phylum Chlorophyta, the class Trebouxiophytae, the order Chlorellales, or the family Chlorellacae. In one embodiment, the microalgae are of the genus Prototheca or Chlorella. In one embodiment, the microalgae are of the species Prototheca moriformis, Prototheca zopfii, Prototheca wickerhamii Prototheca blaschkeae, Prototheca chlorelloides, Prototheca crieana, Prototheca dilamenta, Prototheca hydrocarbonea, Prototheca kruegeri, Prototheca portoricensis, Prototheca salmonis, Prototheca segbwema, Prototheca stagnorum, Prototheca trispora Prototheca ulmea, or Prototheca viscosa. Preferably, the microalga is of the species Prototheca moriformis. In one embodiment, the microalgae are of the species Chlorella autotrophica, Chlorella colonials, Chlorella lewinii, Chlorella minutissima, Chlorella pituitam, Chlorella pulchelloides, Chlorella pyrenoidosa, Chlorella rotunda, Chlorella singularis, Chlorella sorokiniana, Chlorella variabilis, or Chlorella volutis. Preferably, the microalga is of the species Chlorella protothecoides or Auxenochlorella protothecoides. The host cells express the nucleic acids for Embodiments relating to acyltransferases of the invention.

### Embodiment 21

In this embodiment, the nucleic acid encoding the variant Brassica thioesterase encodes a variant thioesterase that has 75%, 80%, 85%, 90%, 95%, 98%, 99%, or 100%, or at least 75%, 80%, 85%, 90%, 95%, 98%, 99%, or 100% identity to SEQ ID NOs: 165, 166, 167, or 168 and comprise one or more of amino acid variants D124A, D209A, D127A or D212A. In another aspect, the nucleic acid encoding the variant Garcinia thioesterase encodes a variant thioesterase that has 75%, 80%, 85%, 90%, 95%, 98%, 99%, or 100%, or at least 75%, 80%, 85%, 90%, 95%, 98%, 99%, or 100% identity to SEQ ID NOs: 137, 138, 139, 140, 141, 142, 143, 144, 145, 146, 147, 148, 149, or 150, and comprise one or more of amino acid variants L91F, L91K, L91S, G96A, G96T, G96V, G108A, G108V, S111A, S111V T156F, T156A, T156K, T156V, or V193A.

### Embodiment 22

In this embodiment, nucleic acids encoding a variant Brassica thioesterase or a variant Garcinia thioesetrase that decrease the production of C18:0 and/or decrease the production of C18:1 fatty acids and/or decreases the production of C18:2 fatty acids sn-2 in the host cell.

### Embodiment 23

In this embodiment, nucleic acids encoding a variant Brassica thioesterase of the invention have SEQ ID NOs: 165, 166, 167, or 168 and comprise one or more of amino acid variants D124A, D209A, D127A or D212A.

### Embodiment 24

In this embodiment, nucleic acids encoding a variant Garcinia thioesetrase of the invention have 75%, 80%, 85%, 90%, 95%, 98%, 99%, or 100%, or at least 75%, 80%, 85%, 90%, 95%, 98%, 99%, or 100% identity to SEQ ID NOs: 137, 138, 139, 140, 141, 142, 143, 144, 145, 146, 147, 148, 149, or 150 and comprise one or more of amino acid variants L91F, L91K, L91S, G96A, G96T, G96V, G108A, G108V, S111A, S111V T156F, T156A, T156K, T156V, or V193A.

### Embodiment 25

This embodiment is a method of cultivating a host cell expressing nucleic acids that encode the one or more acyl transferases of embodiments 16-24.

### Embodiment 26

This embodiment is a method of producing an oil by cultivating host cells that express nucleic acids that encode the one or more variant thioesterases of Embodiments 16-25 and recovering the oil.

### Embodiment 27

This embodiment is an oil produced by cultivating host cells that express the one or more nucleic acids that encode the variant transferases of Examples 16-24, and recovering the oil from the host cell. When the host cell is a microalgae, the cell oil produced by the host cell has sterols that are different than the sterols produced by a plant cell. The cell oil has a sterol profile that is different than an oil obtained from a plant.

### Embodiment 28

In this embodiment, a recombinant variant thioesterase is provided. The recombinant variant thioesterase is produce by a host cell. The glycosylation of the recombinant variant thioesterase is altered from the glycosylation pattern observed in the variant thioesterase produced by the non-recombinant, wild-type cell from which the gene encoding the variant thioesterase was derived.

By way of example and not intended to be the only combination, the acyltransferase and/or the variant acyl-ACP thioesterrases of the invention can be expressed in a cell in which an endogenous desaturase, KAS, and/or fatty acyl-ACP thioesterase has been ablated or downregulated as demonstrated in the Examples. The co-expression of an acyltransferase and/or a variant acyl-ACP thioesterase concomitantly with an invertase is an embodiment of the invention, as was demonstrated in the disclosed Examples. Additionally, the expression of an acyltansferase and/or a variant acyl-ACP thioesterase with concomitant expression of a invertase and ablation or downregulation of a desaturase, KAS and/or fatty acyl-ACP thioesterase is an embodiment of the invention, as demonstrated in the disclosed Examples.

### BRIEF DESCRIPTION OF THE DRAWINGS

FIG. 1. TAG profiles of S7815 versus the S6573 parent. TAGs in brackets co-elute with the peak of the main TAG, but are present in trace amounts, and do not contribute significantly to the area. M=myristate (C14:0), P=palmitate (C16:0), Po=palmitoleate (C16:1), Ma=margaric (C17:0), S=stearate (C18:0), O=oleate (C18:1), L=linoleate (C18:2), Ln=linolenate (C18:3 α), A=arachidate (C20:0), B=behenate (C22:0), Lg=lignocerate (C24:0), Hx=hexacosanoate (C26:0). Sat-Sat-Sat=trisaturates. See Example 5.

FIG. 2. TAG profiles of lipids from fermentations of S7815 versus S6573. TAGs in brackets co-elute with the peak of the main TAG, but are present in trace amounts, and do not contribute significantly to the area. M=myristate (C14:0), P=palmitate (C16:0), S=stearate (C18:0), O=oleate (C18:1), L=linoleate (C18:2), Ln=linolenate (C18:3 α), A=arachidate (C20:0), B=behenate (C22:0), Lg=lignocerate (C24:0), Hx=hexacosanoate (C26:0). Sat-Sat-Sat=trisaturates. See Example 5.

### DETAILED DESCRIPTION OF THE INVENTION

### I. Definitions

An “allele” refers to a copy of a gene where an organism has multiple similar or identical gene copies, even if on the same chromosome. An allele may encode the same or similar protein.

An “oil,” “cell oil” or “cell fat” shall mean a predominantly triglyceride oil obtained from an organism, where the oil has not undergone blending with another natural or synthetic oil, or fractionation so as to substantially alter the fatty acid profile of the triglyceride. In connection with an oil comprising triglycerides of a particular regiospecificity, the cell oil or cell fat has not been subjected to interesterification or other synthetic process to obtain that regiospecific triglyceride profile, rather the regiospecificity is produced naturally, by a cell or population of cells. For a cell oil produced by a cell, the sterol profile of oil is generally determined by the sterols produced by the cell, not by artificial reconstitution of the oil by adding sterols in order to mimic the cell oil. In connection with a cell oil or cell fat, and as used generally throughout the present disclosure, the terms oil, and fat are used interchangeably, except where otherwise noted. Thus, an “oil” or a “fat” can be liquid, solid, or partially solid at room temperature, depending on the makeup of the substance and other conditions. Here, the term “fractionation” means removing material from the oil in a way that changes its fatty acid profile relative to the profile produced by the organism, however accomplished. The terms “oil,” “cell oil” and “cell fat” encompass such oils obtained from an organism, where the oil has undergone minimal processing, including refining, bleaching, deodorized, and/or degumming, which does not substantially change its triglyceride profile. A cell oil can also be a “noninteresterified cell oil”, which means that the cell oil has not undergone a process in which fatty acids have been redistributed in their acyl linkages to glycerol and remain essentially in the same configuration as when recovered from the organism.

As used herein, an oil is said to be “enriched” in one or more particular fatty acids if there is at least a 10% increase in the mass of that fatty acid in the oil relative to the non-enriched oil. For example, in the case of a cell expressing a heterologous FatB gene described herein, the oil produced by the cell is said to be enriched in, e.g., C8 and C16 fatty acids if the mass of these fatty acids in the oil is at least 10% greater than in oil produced by a cell of the same type that does not express the heterologous FatB gene (e.g., wild type oil).

“Exogenous gene” shall mean a nucleic acid that codes for the expression of an RNA and/or protein that has been introduced into a cell (e.g. by transformation/transfection), and is also referred to as a “transgene”. A cell comprising an exogenous gene may be referred to as a recombinant cell, into which additional exogenous gene(s) may be introduced. The exogenous gene may be from a different species (and so heterologous), or from the same species (and so homologous), relative to the cell being transformed. Thus, an exogenous gene can include a homologous gene that occupies a different location in the genome of the cell or is under different control, relative to the endogenous copy of the gene. An exogenous gene may be present in more than one copy in the cell. An exogenous gene may be maintained in a cell as an insertion into the genome (nuclear or plastid) or as an episomal molecule.

“FADc”, also referred to as “FAD2” or “FAD” is a gene encoding a delta-12 fatty acid desaturase. “SAD” is a gene encoding a stearoyl ACP desaturase, a delta-9 fatty acid desaturase. The desaturases desaturates a fatty acyl chain to create a double bond. SAD converts stearic acid, C18:0 to oleic acid, C18:1 and FAD converts oleic acid, C18:1 to linoleic acid, C18:2.

“Fatty acids” shall mean free fatty acids, fatty acid salts, or fatty acyl moieties in a glycerolipid. It will be understood that fatty acyl groups of glycerolipids can be described in terms of the carboxylic acid or anion of a carboxylic acid that is produced when the triglyceride is hydrolyzed or saponified.

“Fixed carbon source” is a molecule(s) containing carbon, typically an organic molecule that is present at ambient temperature and pressure in solid or liquid form in a culture media that can be utilized by a microorganism cultured therein. Accordingly, carbon dioxide is not a fixed carbon source. Typical fixed carbon source include sucrose, glucose, fructose and other well-known monosaccharides, disaccharides and polysaccharides.

“In operable linkage” is a functional linkage between two nucleic acid sequences, such a control sequence (typically a promoter) and the linked sequence (typically a sequence that encodes a protein, also called a coding sequence). A promoter is in operable linkage with an exogenous gene if it can mediate transcription of the gene.

“Microalgae” are eukaryotic microbial organisms that contain a chloroplast or other plastid, and optionally that is capable of performing photosynthesis, or a prokaryotic microbial organism capable of performing photosynthesis. Microalgae include obligate photoautotrophs, which cannot metabolize a fixed carbon source as energy, as well as heterotrophs, which can live solely off of a fixed carbon source. Microalgae also include mixotrophic organisms that can perform photosynthesis and metabolize one or more fixed carbon source. Microalgae include unicellular organisms that separate from sister cells shortly after cell division, such as Chlamydomonas, as well as microbes such as, for example, volvox, which is a simple multicellular photosynthetic microbe of two distinct cell types. Microalgae include cells such as Chlorella, Dunaliella, and Prototheca. Microalgae also include other microbial photosynthetic organisms that exhibit cell-cell adhesion, such as Agmenellum, Anabaena, and Pyrobotrys. Microalgae also include obligate heterotrophic microorganisms that have lost the ability to perform photosynthesis, such as certain dinoflagellate algae species and species of the genus Prototheca.

As used with respect to nucleic acids, the term “isolated” refers to a nucleic acid that is free of at least one other component that is typically present with the naturally occurring nucleic acid. Thus, a naturally occurring nucleic acid is isolated if it has been purified away from at least one other component that occurs naturally with the nucleic acid.

In connection with fatty acid length, “mid-chain” shall mean C8 to C16 fatty acids.

In connection with a recombinant cell, the term “knockdown” refers to a gene that has been partially suppressed (e.g., by about 1-95%) in terms of the production or activity of a protein encoded by the gene. Inhibitory RNA technology to down-regulate or knockdown expression of a gene are well known. These techniques include dsRNA, hairpin RNA, antisense RNA, interfering RNA (RNAi) and others.

Also, in connection with a recombinant cell, the term “knockout” refers to a gene that has been completely or nearly completely (e.g., >95%) suppressed in terms of the production or activity of a protein encoded by the gene. Knockouts can be prepared by ablating the gene by homologous recombination of a nucleic acid sequence into a coding sequence, gene deletion, mutation or other method. When homologous recombination is performed, the nucleic acid that is inserted (“knocked-in”) can be a sequence that encodes an exogenous gene of interest or a sequence that does not encode for a gene of interest. The ablation by homologous recombination can be performed in one, two or more alleles of the gene of interest.

An “oleaginous” cell is a cell capable of producing at least 20% lipid by dry cell weight, naturally or through recombinant or classical strain improvement. An “oleaginous microbe” or “oleaginous microorganism” is a microbe, including a microalga that is oleaginous (especially eukaryotic microalgae that store lipid). An oleaginous cell also encompasses a cell that has had some or all of its lipid or other content removed, and both live and dead cells.

An “ordered oil” or “ordered fat” is one that forms crystals that are primarily of a given polymorphic structure. For example, an ordered oil or ordered fat can have crystals that are greater than 50%, 60%, 70%, 80%, or 90% of the 13 or 13′ polymorphic form.

In connection with a cell oil, a “profile” is the distribution of particular species or triglycerides or fatty acyl groups within the oil. A “fatty acid profile” is the distribution of fatty acyl groups in the triglycerides of the oil without reference to attachment to a glycerol backbone. Fatty acid profiles are typically determined by conversion to a fatty acid methyl ester (FAME), followed by gas chromatography (GC) analysis with flame ionization detection (FID), as in Example 1. The fatty acid profile can be expressed as one or more percent of a fatty acid in the total fatty acid signal determined from the area under the curve for that fatty acid. FAME-GC-FID measurement approximate weight percentages of the fatty acids. A “sn-2 profile” is the distribution of fatty acids found at the sn-2 position of the triacylglycerides in the oil. A “regiospecific profile” is the distribution of triglycerides with reference to the positioning of acyl group attachment to the glycerol backbone without reference to stereospecificity. In other words, a regiospecific profile describes acyl group attachment at sn-1/3 vs. sn-2. Thus, in a regiospecific profile, POS (palmitate-oleate-stearate) and SOP (stearate-oleate-palmitate) are treated identically. A “stereospecific profile” describes the attachment of acyl groups at sn-1, sn-2 and sn-3. Unless otherwise indicated, triglycerides such as SOP and POS are to be considered equivalent. A “TAG profile” is the distribution of fatty acids found in the triglycerides with reference to connection to the glycerol backbone, but without reference to the regiospecific nature of the connections. Thus, in a TAG profile, the percent of SSO in the oil is the sum of SSO and SOS, while in a regiospecific profile, the percent of SSO is calculated without inclusion of SOS species in the oil. In contrast to the weight percentages of the FAME-GC-FID analysis, triglyceride percentages are typically given as mole percentages; that is the percent of a given TAG molecule in a TAG mixture.

The term “percent sequence identity,” in the context of two or more amino acid or nucleic acid sequences, refers to two or more sequences or subsequences that are the same or have a specified percentage of amino acid residues or nucleotides that are the same, when compared and aligned for maximum correspondence, as measured using a sequence comparison algorithm or by visual inspection. For sequence comparison to determine percent nucleotide or amino acid identity, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted using the NCBI BLAST software (ncbi.nlm.nih.gov/BLAST/) set to default parameters. For example, to compare two nucleic acid sequences, one may use blastn with the “BLAST 2 Sequences” tool Version 2.0.12 (Apr. 21, 2000) set at the following default parameters: Matrix: BLOSUM62; Reward for match: 1; Penalty for mismatch: −2; Open Gap: 5 and Extension Gap: 2 penalties; Gap x drop-off: 50; Expect: 10; Word Size: 11; Filter: on. For a pairwise comparison of two amino acid sequences, one may use the “BLAST 2 Sequences” tool Version 2.0.12 (Apr. 21, 2000) with blastp set, for example, at the following default parameters: Matrix: BLOSUM62; Open Gap: 11 and Extension Gap: 1 penalties; Gap x drop-off 50; Expect: 10; Word Size: 3; Filter: on.

“Recombinant” is a cell, nucleic acid, protein or vector that has been modified due to the introduction of an exogenous nucleic acid or the alteration of a native nucleic acid. Thus, e.g., recombinant cells can express genes that are not found within the native (non-recombinant) form of the cell or express native genes differently than those genes are expressed by a non-recombinant cell. Recombinant cells can, without limitation, include recombinant nucleic acids that encode for a gene product or for suppression elements such as mutations, knockouts, antisense, interfering RNA (RNAi), hairpin RNA or dsRNA that reduce the levels of active gene product in a cell. A “recombinant nucleic acid” is a nucleic acid originally formed in vitro, in general, by the manipulation of nucleic acid, e.g., using polymerases, ligases, exonucleases, and endonucleases, using chemical synthesis, or otherwise is in a form not normally found in nature. Recombinant nucleic acids may be produced, for example, to place two or more nucleic acids in operable linkage. Thus, an isolated nucleic acid or an expression vector formed in vitro by ligating DNA molecules that are not normally joined in nature, are both considered recombinant for the purposes of this invention. Once a recombinant nucleic acid is made and introduced into a host cell or organism, it may replicate using the in vivo cellular machinery of the host cell; however, such nucleic acids, once produced recombinantly, although subsequently replicated intracellularly, are still considered recombinant for purposes of this invention. Similarly, a “recombinant protein” is a protein made using recombinant techniques, i.e., through the expression of a recombinant nucleic acid. A recombinant protein will have a different pattern of glycosylation than the protein isolated from the wild-type organism.

The genes can be used in a variety of genetic constructs including plasmids or other vectors for expression or recombination in a host cell. The genes can be codon optimized for expression in a target host cell. The proteins produced by the genes can be used in vivo or in purified form.

For example, the gene can be prepared in an expression vector comprising an operably linked promoter and 5′UTR. Where a plastidic cell is used as the host, a suitably active plastid targeting peptide can be fused to the FATB gene, as in the examples below. Generally, for the newly identified FATB genes, there are roughly 50 amino acids at the N-terminal that constitute a plastid transit peptide, which are responsible for transporting the enzyme to the chloroplast. In the examples below, this transit peptide is replaced with a 38 amino acid sequence that is effective in the Prototheca moriformis host cell for transporting the enzyme to the plastids of those cells. Thus, the invention contemplates deletions and fusion proteins in order to optimize enzyme activity in a given host cell. For example, a transit peptide from the host or related species may be used instead of that of the newly discovered plant genes described here.

A selectable marker gene may be included in the vector to assist in isolating a transformed cell. Examples of selectable markers useful in microlagae include sucrose invertase antibiotic resistance genes and other genes useful as selectable markers. The S. carlbergensis MEL1 gene (conferring the ability to grow on melibiose), A. thaliana THIC gene (conferring the ability to grow in media free of thiamine, Saccharomyces sucrose invertase (conferring the ability to grow on sucrose) are disclosed in the Examples. Other known selectable markers are useful and within the ambit of a skilled artisan.

The terms “triglyceride”, “triacylglyceride” and “TAG” are used interchangeably as is known in the art.

### II. Embodiments of the Invention

Illustrative embodiments of the present invention feature oleaginous cells that produce altered fatty acid profiles and/or altered regiospecific distribution of fatty acids in glycerolipids, and products produced from the cells. Examples of oleaginous cells include microbial cells having a type II fatty acid biosynthetic pathway, including plastidic oleaginous cells such as those of oleaginous algae and, where applicable, oil producing cells of higher plants including but not limited to commercial oilseed crops such as soy, corn, rapeseed/canola, cotton, flax, sunflower, safflower and peanut. Other specific examples of cells include heterotrophic or obligate heterotrophic microalgae of the phylum Chlorophtya, the class Trebouxiophytae, the order Chlorellales, or the family Chlorellacae. Examples of oleaginous microalgae and methods of cultivation are also provided in co-owned applications WO2008/151149, WO2010/063031, WO2010/063032, WO2011/150410, WO2011/150411, WO2012/061647, WO2012/061647, WO2012/106560, and WO2013/158938, WO2014/120829, WO2014/151904, WO2015/051319, WO2016/007862, WO2016/014968, WO2016/044779, WO2016/164495, all of which are incorporated by reference, including species of Chlorella and Prototheca, a genus comprising obligate heterotrophs. The oleaginous cells can be, for example, capable of producing 25%, 30%, 40%, 50%, 60%, 70%, 80%, 85%, or about 90% oil by cell weight, ±5%. Optionally, the oils produced can be low in highly unsaturated fatty acids such as DHA or EPA fatty acids. For example, the oils can comprise less than 5%, 2%, or 1% DHA and/or EPA. The above-mentioned publications also disclose methods for cultivating such cells and extracting oil, especially from microalgal cells; such methods are applicable to the cells disclosed herein and incorporated by reference for these teachings. When microalgal cells are used they can be cultivated autotrophically (unless an obligate heterotroph) or in the dark using a sugar (e.g., glucose, fructose and/or sucrose) In any of the embodiments described herein, the cells can be heterotrophic cells comprising an exogenous invertase gene so as to allow the cells to produce oil from a sucrose feedstock. Alternately, or in addition, the cells can metabolize xylose from cellulosic feedstocks. For example, the cells can be genetically engineered to express one or more xylose metabolism genes such as those encoding an active xylose transporter, a xylulose-5-phosphate transporter, a xylose isomerase, a xylulokinase, a xylitol dehydrogenase and a xylose reductase. See WO2012/154626, “GENETICALLY ENGINEERED MICROORGANISMS THAT METABOLIZE XYLOSE”, published Nov. 15, 2012, including disclosure of genetically engineered Prototheca strains that utilize xylose.

The host cells expressing the acyltransferases or the variant B. napus thioesterases or the variant G. mangostana thioesterase may, optionally, be cultivated in a bioreactor/fermenter. For example, heterotrophic oleaginous microalgal cells can be cultivated on a sugar-containing nutrient broth. Optionally, cultivation can proceed in two stages: a seed stage and a lipid-production stage. In the seed stage, the number of cells is increased from a starter culture. Thus, the seed stage(s) typically includes a nutrient rich, nitrogen replete, media designed to encourage rapid cell division. After the seed stage(s), the cells may be fed sugar under nutrient-limiting (e.g. nitrogen sparse) conditions so that the sugar will be converted into triglycerides. As used herein, “standard lipid production conditions” are disclosed here. In one embodiment, the culture conditions are nitrogen limiting. Sugar and other nutrients can be added during the fermentation but no additional nitrogen is added. The cells will consume all or nearly all of the nitrogen present, but no additional nitrogen is provided. For example, the rate of cell division in the lipid-production stage can be decreased by 50%, 80%, or more relative to the seed stage. Additionally, variation in the media between the seed stage and the lipid-production stage can induce the recombinant cell to express different lipid-synthesis genes and thereby alter the triglycerides being produced. For example, as discussed below, nitrogen and/or pH sensitive promoters can be placed in front of endogenous or exogenous genes. This is especially useful when an oil is to be produced in the lipid-production phase that does not support optimal growth of the cells in the seed stage.

The oleaginous cells express one or more exogenous genes encoding fatty acid biosynthesis enzymes. As a result, some embodiments feature cell oils that were not obtainable from a non-plant or non-seed oil, or not obtainable at all.

The oleaginous cells, including microalgal cells, can be improved via classical strain improvement techniques such as UV and/or chemical mutagenesis followed by screening or selection under environmental conditions, including selection on a chemical or biochemical toxin. For example the cells can be selected on a fatty acid synthesis inhibitor, a sugar metabolism inhibitor, or an herbicide. As a result of the selection, strains can be obtained with increased yield on sugar, increased oil production (e.g., as a percent of cell volume, dry weight, or liter of cell culture), or improved fatty acid or TAG profile. Co-owned application PCT/US2016/025023 filed on 31 Mar. 2016, herein incorporated by reference, describes methods for classically mutagenizing oleaginous cells.

The cells can be selected on one or more of 1,2-Cyclohexanedione; 19-Norethindone acetate; 2,2-dichloropropionic acid; 2,4,5-trichlorophenoxyacetic acid; 2,4,5-trichlorophenoxyacetic acid, methyl ester; 2,4-dichlorophenoxyacetic acid; 2,4-dichlorophenoxyacetic acid, butyl ester; 2,4-dichlorophenoxyacetic acid, isooctyl ester; 2,4-dichlorophenoxyacetic acid, methyl ester; 2,4-dichlorophenoxybutyric acid; 2,4-dichlorophenoxybutyric acid, methyl ester; 2,6-dichlorobenzonitrile; 2-deoxyglucose; 5-Tetradecyloxy-w-furoic acid; A-922500; acetochlor; alachlor; ametryn; amphotericin; atrazine; benfluralin; bensulide; bentazon; bromacil; bromoxynil; Cafenstrole; carbonyl cyanide m-chlorophenyl hydrazone (CCCP); carbonyl cyanide-p-trifluoromethoxyphenylhydrazone (FCCP); cerulenin; chlorpropham; chlorsulfuron; clofibric acid; clopyralid; colchicine; cycloate; cyclohexamide; C75; DACTHAL (dimethyl tetrachloroterephthalate); dicamba; dichloroprop ((R)-2-(2,4-dichlorophenoxy)propanoic acid); Diflufenican; dihyrojasmonic acid, methyl ester; diquat; diuron; dimethylsulfoxide; Epigallocatechin gallate (EGCG); endothall; ethalfluralin; ethanol; ethofumesate; Fenoxaprop-p-ethyl; Fluazifop-p-Butyl; fluometuron; fomasefen; foramsulfuron; gibberellic acid; glufosinate ammonium; glyphosate; haloxyfop; hexazinone; imazaquin; isoxaben; Lipase inhibitor THL ((−)-Tetrahydrolipstatin); malonic acid; MCPA (2-methyl-4-chlorophenoxyacetic acid); MCPB (4-(4-chloro-o-tolyloxy)butyric acid); mesotrione; methyl dihydrojasmonate; metolachlor; metribuzin; Mildronate; molinate; naptalam; norharman; orlistat; oxadiazon; oxyfluorfen; paraquat; pendimethalin; pentachlorophenol; PF-04620110; phenethyl alcohol; phenmedipham; picloram; Platencin; Platensimycin; prometon; prometryn; pronamide; propachlor; propanil; propazine; pyrazon; Quizalofop-p-ethyl; s-ethyl dipropylthiocarbamate (EPTC); s,s,s-tributylphosphorotrithioate; salicylhydroxamic acid; sesamol; siduron; sodium methane arsenate; simazine; T-863 (DGAT inhibitor); tebuthiuron; terbacil; thiobencarb; tralkoxydim; triallate; triclopyr; triclosan; trifluralin; and vulpinic acid and others.

The oleaginous cells produce a storage oil, which is primarily triacylglyceride and may be stored in storage bodies of the cell. A raw oil may be obtained from the cells by disrupting the cells and isolating the oil. The raw oil may comprise sterols produced by the cells. Patent applications WO2008/151149, WO2010/063031, WO2010/063032, WO2011/150410, WO2011/150411, WO2012/061647, WO2012/061647, WO2012/106560, WO2013/158938, WO2014/120829, WO2014/151904, WO2015/051319, WO2016/007862, WO2016/014968, WO2016/044779, and WO2016/164495 disclose heterotrophic cultivation and oil isolation techniques for oleaginous microalgae. For example, oil may be obtained by providing or cultivating, drying and pressing the cells. The oils produced may be refined, bleached and deodorized (RBD) as known in the art or as described in WO2010/120939. The raw or RBD oils may be used in a variety of food, chemical, and industrial products or processes. Even after such processing, the oil may retain a sterol profile characteristic of the source. Sterol profiles of microalga and the microalgal cell oils are disclosed below. After recovery of the oil, a valuable residual biomass remains. Uses for the residual biomass include the production of paper, plastics, absorbents, adsorbents, drilling fluids, as animal feed, for human nutrition, or for fertilizer.

In an embodiment of the invention nucleic acids that encode novel acyl transferases are provided. The novel acyltransferases are useful in altering the fatty acid profile and/or altering the regiospecific profile of an oil produced by a host cell. The nucleic acids of the invention may contain control sequences upstream and downstream in operable linkage with the gene of interest. These control sequences include promoters, targeting sequences, untranslated sequences and other control elements. Nucleic acids of the invention encode acyltransferases that function in type II fatty acid synthesis. The acyltransferase genes are isolated from higher plants and can be expressed in a wide variety of host cells. The acyltransferases include lysophosphatidic acid acyltransferase (LPAAT), glycerol phosphate acyltransferase (GPAT), diacyl glycerol acyltransferase (DGAT), lysophosphatidylcholine acyltransferase (LPCAT), or phospholipase A2 (PLA2). and other lipid biosynthetic pathway genes as discussed herein. The acyltransferases of the invention are shown in Table 5. In one embodiment, the acyltransferases of the invention have acyltransferase activity and the amino acid sequence comprises at least 96.3%, 98%, or 99% identity to an acyltransferase of clade 1 of Table 5. In another embodiment, the acyltransferases of the invention have acyltransferase activity and the amino acid sequence comprises at least 93.9%, 98%, or 99% identity to an acyltransferase of clade 2 of Table 5. In one embodiment, the acyltransferases of the invention have acyltransferase activity and the amino acid sequence comprises at least 86.5%, 90%, 95%, 98%, or 99% identity to an acyltransferase of clade 3 of Table 5. In one embodiment, the acyltransferases of the invention have acyltransferase activity and the amino acid sequence comprises at least 78.5%, 80%, 85%, 90%, 95%, 98%, or 99% identity to an acyltransferase of clade 4 of Table 5. The acyltransferases when expressed increase the SOS, POP, POS, SLS, PLO, and/or PLO content DCW in host cells and the oils recovered from the host cells. The acyltransferases when expressed in host cells decreases the sat-sat-sat content of the oil by DCW. The acyltransferases when expressed in host cells increases the sat-unsat-sat/sat-sat-sat ratio of the oil by DCW.

In an embodiment of the invention nucleic acids that encode variant Brassica napus thiosterases (FATA) are provided. The novel thioesterases are useful in altering the fatty acid profile of an oil produced by a host cell. The variant Brassica napus thiosterases prefer to hydrolyze long chain fatty acyl groups from the acyl carrier protein. The nucleic acids of the invention may contain control sequences upstream and downstream in operable linkage with the gene of interest. These control sequences include promoters, targeting sequences, untranslated sequences and other control elements. Nucleic acids of the invention encode thiosterases that function in type II fatty acid synthesis. The thioesterase genes, isolated from higher plants, are altered to create variant thioesterases that have certain amino acids that have been altered from the wild type enzyme. Due to the altered amino acid(s), the substrate specificity of the thioesterase is altered. The variant thioesterases can be expressed in a wide variety of host cells. The nucleic acids encode the variant thioesterases having amino acid sequences that are 75%, 80%, 85%, 90%, 95%, 98%, 99%, or 100%, or at least 75%, 80%, 85%, 90%, 95%, 98%, 99%, or 100% identical to SEQ ID NOs: 165, 166, 167, or 198_and comprise one or more of amino acid variants D124A, D209A, D127A or D212A. The variant BnOTE enzymes increased C18:0 content by DCW, decreased C18:1 content by DCW, and decreased C18:2 content by DCW in host cells and the oils recovered from the host cells.

In an embodiment of the invention nucleic acids that encode variant Garcinia mangostana thiosterases (FATA) are provided. The novel thioesterases are useful in altering the fatty acid profile of an oil produced by a host cell. The variant Garcinia mangostana thiosterases prefer to hydrolyze long chain fatty acyl groups from the acyl carrier protein. The nucleic acids of the invention may contain control sequences upstream and downstream in operable linkage with the gene of interest. These control sequences include promoters, targeting sequences, untranslated sequences and other control elements. Nucleic acids of the invention encode thiosterases that function in type II fatty acid synthesis. The thioesterase genes, isolated from higher plants, are altered to create variant thioesterases that have certain amino acids that have been altered from the wild type enzyme. Due to the altered amino acid(s), the substrate specificity of the thioesterase is altered. The variant thioesterases can be expressed in a wide variety of host cells. The nucleic acids encode the variant thioesterases having amino acid sequences that are 75%, 80%, 85%, 90%, 95%, 98%, 99%, or 100%, or at least 75%, 80%, 85%, 90%, 95%, 98%, 99%, or 100% identical to SEQ ID NOs: 137, 138, 139, 140, 141, 142, 143, 144, 145, 146, 147, 148, 149, or 150 and comprise one or more of amino acid variants L91F, L91K, L91S, G96A, G96T, G96V, G108A, G108V, S111A, S111V T156F, T156A, T156K, T156V, or V193A. The variant GmFATA enzymes increased C18:0 content by DCW, decreased C18:1 content by DCW, and decreased C18:2 content by DCW in host cells and the oils recovered from the host cells.

The nucleic acids of the invention can be codon optimized for expression in a target host cell (e.g., using the codon usage tables of Tables 1a, 1b, 2a, and 2b. For example, at least 60%, 65%, 70%, 75%, 80%, 85%, 90%, 95%, or 100% of the codons used can be the most preferred codon according to Tables 1a, 1b, 2a, and 2b. Alternately, at least 60%, 65%, 70%, 75%, 80%, 85%, 90%, 95%, or 100% of the codons used can be the first or second most preferred codon according to Tables 1a, 1b, 2a, and 2b. Preferred codons for Prototheca strains and for Chlorella protothecoides are shown below in Tables 1a and 1b, respectively.

TABLE 1a
Preferred codon usage in Prototheca strains.

| | | | | | | | |
|---|---|---|---|---|---|---|---|
| Ala | GCG | 345 | (0.36) | Asn | AAT | 8 | (0.04) |
| | GCA | 66 | (0.07) | | AAC | 201 | (0.96) |
| | GCT | 101 | (0.11) | | | | |
| | GCC | 442 | (0.46) | Pro | CCG | 161 | (0.29) |
| | | | | | CCA | 49 | (0.09) |
| Cys | TGT | 12 | (0.10) | | CCT | 71 | (0.13) |
| | TGC | 105 | (0.90) | | CCC | 267 | (0.49) |
| Asp | GAT | 43 | (0.12) | Gln | CAG | 226 | (0.82) |
| | GAC | 316 | (0.88) | | CAA | 48 | (0.18) |
| Glu | GAG | 377 | (0.96) | Arg | AGG | 33 | (0.06) |
| | GAA | 14 | (0.04) | | AGA | 14 | (0.02) |
| | | | | | CGG | 102 | (0.18) |
| Phe | TTT | 89 | (0.29) | | CGA | 49 | (0.08) |
| | TTC | 216 | (0.71) | | CGT | 51 | (0.09) |
| | | | | | CGC | 331 | (0.57) |
| Gly | GGG | 92 | (0.12) | | | | |
| | GGA | 56 | (0.07) | Ser | AGT | 16 | (0.03) |
| | GGT | 76 | (0.10) | | AGC | 123 | (0.22) |
| | GGC | 559 | (0.71) | | TCG | 152 | (0.28) |
| | | | | | TCA | 31 | (0.06) |
| His | CAT | 42 | (0.21) | | TCT | 55 | (0.10) |
| | CAC | 154 | (0.79) | | TCC | 173 | (0.31) |
| Ile | ATA | 4 | (0.01) | Thr | ACG | 184 | (0.38) |
| | ATT | 30 | (0.08) | | ACA | 24 | (0.05) |
| | ATC | 338 | (0.91) | | ACT | 21 | (0.05) |
| | | | | | ACC | 249 | (0.52) |
| Lys | AAG | 284 | (0.98) | | | | |
| | AAA | 7 | (0.02) | Val | GTG | 308 | (0.50) |
| | | | | | GTA | 9 | (0.01) |
| Leu | TTG | 26 | (0.04) | | GTT | 35 | (0.06) |
| | TTA | 3 | (0.00) | | GTC | 262 | (0.43) |
| | CTG | 447 | (0.61) | | | | |
| | CTA | 20 | (0.03) | Trp | TGG | 107 | (1.00) |
| | CTT | 45 | (0.06) | | | | |
| | CTC | 190 | (0.26) | Tyr | TAT | 10 | (0.05) |
| | | | | | TAC | 180 | (0.95) |
| Met | ATG | 191 | (1.00) | | | | |

| | |
|---|---|
| Stop | TGA/TAG/TAA |

TABLE 1b
Preferred codon usage in Chlorella protothecoides.

| | | | |
|---|---|---|---|
| TTC (Phe) | TAC (Tyr) | TGC (Cys) | TGA (Stop) |
| TGG (Trp) | CCC (Pro) | CAC (His) | CGC (Arg) |
| CTG (Leu) | CAG (Gln) | ATC (Ile) | ACC (Thr) |
| GAC (Asp) | TCC (Ser) | ATG (Met) | AAG (Lys) |
| GCC (Ala) | AAC (Asn) | GGC (Gly) | GTG (Val) |
| GAG (Glu) | | | |

TABLE 2a
Codon usage for Cuphea wrightii

UUU F 0.48 19.5 (52) UCU S 0.21 19.5 (52) UAU Y 0.45 6.4 (17) UGU C 0.41 10.5 (28)
UUC F 0.52 21.3 (57) UCC S 0.26 23.6 (63) UAC Y 0.55 7.9 (21) UGC C 0.59 15.0 (40)
UUA L 0.07 5.2 (14) UCA S 0.18 16.8 (45) UAA * 0.33 0.7 (2) UGA * 0.33 0.7 (2)
UUG L 0.19 14.6 (39) UCG S 0.11 9.7 (26) UAG * 0.33 0.7 (2) UGG W 1.00 15.4 (41)
CUU L 0.27 21.0 (56) CCU P 0.48 21.7 (58) CAU H 0.60 11.2 (30) CGU R 0.09 5.6 (15)
CUC L 0.22 17.2 (46) CCC P 0.16 7.1 (19) CAC H 0.40 7.5 (20) CGC R 0.13 7.9 (21)
CUA L 0.13 10.1 (27) CCA P 0.21 9.7 (26) CAA Q 0.31 8.6 (23) CGA R 0.11 6.7 (18)
CUG L 0.12 9.7 (26) CCG P 0.16 7.1 (19) CAG Q 0.69 19.5 (52) CGG R 0.16 9.4 (25)
AUU I 0.44 22.8 (61) ACU T 0.33 16.8 (45) AAU N 0.66 31.4 (84) AGU S 0.18 16.1 (43)
AUC I 0.29 15.4 (41) ACC T 0.27 13.9 (37) AAC N 0.34 16.5 (44) AGC S 0.07 6.0 (16)
AUA I 0.27 13.9 (37) ACA T 0.26 13.5 (36) AAA K 0.42 21.0 (56) AGA R 0.24 14.2 (38)
AUG M 1.00 28.1 (75) ACG T 0.14 7.1 (19) AAG K 0.58 29.2 (78) AGG R 0.27 16.1 (43)
GUU V 0.28 19.8 (53) GCU A 0.35 31.4 (84) GAU D 0.63 35.9 (96) GGU G 0.29 26.6 (71)
GUC V 0.21 15.0 (40) GCC A 0.20 18.0 (48) GAC D 0.37 21.0 (56) GGC G 0.20 18.0 (48)
GUA V 0.14 10.1 (27) GCA A 0.33 29.6 (79) GAA E 0.41 18.3 (49) GGA G 0.35 31.4 (84)
GUG V 0.36 25.1 (67) GCG A 0.11 9.7 (26) GAG E 0.59 26.2 (70) GGG G 0.16 14.2 (38)

TABLE 2b
Codon usage for Arabidopsis

UUU F 0.51 21.8 (678320) UCU S 0.28 25.2 (782818) UAU Y 0.52 14.6 (455089) UGU C 0.60 10.5
(327640)
UUC F 0.49 20.7 (642407) UCC S 0.13 11.2 (348173) UAC Y 0.48 13.7 (427132) UGC C 0.40 7.2 (222769)
UUA L 0.14 12.7 (394867) UCA S 0.20 18.3 (568570) UAA * 0.36 0.9 (29405) UGA * 0.44 1.2 (36260)
UUG L 0.22 20.9 (649150) UCG S 0.10 9.3 (290158) UAG * 0.20 0.5 (16417) UGG W 1.00 12.5 (388049)
CUU L 0.26 24.1 (750114) CCU P 0.38 18.7 (580962) CAU H 0.61 13.8 (428694) CGU R 0.17 9.0 (280392)
CUC L 0.17 16.1 (500524) CCC P 0.11 5.3 (165252) CAC H 0.39 8.7 (271155) CGC R 0.07 3.8 (117543)
CUA L 0.11 9.9 (307000) CCA P 0.33 16.1 (502101) CAA Q 0.56 19.4 (604800) CGA R 0.12 6.3 (195736)
CUG L 0.11 9.8 (305822) CCG P 0.18 8.6 (268115) CAG Q 0.44 15.2 (473809) CGG R 0.09 4.9 (151572)
AUU I 0.41 21.5 (668227) ACU T 0.34 17.5 (544807) AAU N 0.52 22.3 (693344) AGU S 0.16 14.0 (435738)
AUC I 0.35 18.5 (576287) ACC T 0.20 10.3 (321640) AAC N 0.48 20.9 (650826) AGC S 0.13 11.3 (352568)
AUA I 0.24 12.6 (391867) ACA T 0.31 15.7 (487161) AAA K 0.49 30.8 (957374) AGA R 0.35 19.0 (589788)
AUG M 1.00 24.5 (762852) ACG T 0.15 7.7 (240652) AAG K 0.51 32.7 (1016176) AGG R 0.20 11.0
(340922)
GUU V 0.40 27.2 (847061) GCU A 0.43 28.3 (880808) GAU D 0.68 36.6 (1139637) GGU G 0.34 22.2
(689891)
GUC V 0.19 12.8 (397008) GCC A 0.16 10.3 (321500) GAC D 0.32 17.2 (535668) GGC G 0.14 9.2
(284681)
GUA V 0.15 9.9 (308605) GCA A 0.27 17.5 (543180) GAA E 0.52 34.3 (1068012) GGA G 0.37 24.2
(751489)
GUG V 0.26 17.4 (539873) GCG A 0.14 9.0 (280804) GAG E 0.48 32.2 (1002594) GGG G 0.16 10.2
(316620)

The cell oils of this invention can be distinguished from conventional vegetable or animal triacylglycerol sources in that the sterol profile will be indicative of the host organism as distinguishable from the conventional source. Conventional sources of oil include soy, corn, sunflower, safflower, palm, palm kernel, coconut, cottonseed, canola, rape, peanut, olive, flax, tallow, lard, cocoa, shea, mango, sal, illipe, kokum, and allanblackia.

The oils provided herein are not vegetable oils. Vegetable oils are oils extracted from plants and plant seeds. Vegetable oils can be distinguished from the non-plant oils provided herein on the basis of their oil content. A variety of methods for analyzing the oil content can be employed to determine the source of the oil or whether adulteration of an oil provided herein with an oil of a different (e.g. plant) origin has occurred. The determination can be made on the basis of one or a combination of the analytical methods. These tests include but are not limited to analysis of one or more of free fatty acids, fatty acid profile, total triacylglycerol content, diacylglycerol content, peroxide values, spectroscopic properties (e.g. UV absorption), sterol profile, sterol degradation products, antioxidants (e.g. tocopherols), pigments (e.g. chlorophyll), d13C values and sensory analysis (e.g. taste, odor, and mouth feel). Many such tests have been standardized for commercial oils such as the Codex Alimentarius standards for edible fats and oils.

Sterol profile analysis is a particularly well-known method for determining the biological source of organic matter. Campesterol, b-sitosterol, and stigamsterol are common plant sterols, with b-sitosterol being a principle plant sterol. For example, b-sitosterol was found to be in greatest abundance in an analysis of certain seed oils, approximately 64% in corn, 29% in rapeseed, 64% in sunflower, 74% in cottonseed, 26% in soybean, and 79% in olive oil (Gul et al. J. Cell and Molecular Biology 5:71-79, 2006).

The sterol profile of a microalgal oil is distinct from the sterol profile of oils obtained from higher plants or animals. Oil isolated from Prototheca moriformis strain UTEX1435 were separately clarified (CL), refined and bleached (RB), or refined, bleached and deodorized (RBD) and were tested for sterol content according to the procedure described in JAOCS vol. 60, no. 8, August 1983. Results of the analysis are shown Table 3 below (units in mg/100 g):

TABLE 3
(units in mg/100 g)

| | | | | | Refined, |
|---|---|---|---|---|---|
| | | | | Refined & | bleached, & |
| | Sterol | Crude | Clarified | bleached | deodorized |
| 1 | Ergosterol | 384 | 398 | 293 | 302 |
| | | (56%) | (55%) | (50%) | (50%) |
| 2 | 5,22-cholestadien-24- | 14.6 | 18.8 | 14 | 15.2 |
| | methyl-3-ol | (2.1%) | (2.6%) | (2.4%) | (2.5%) |
| | (Brassicasterol) | | | | |
| 3 | 24-methylcholest-5- | 10.7 | 11.9 | 10.9 | 10.8 |
| | en-3-ol (Campesterol or | (1.6%) | (1.6%) | (1.8%) | (1.8%) |
| | 22,23- | | | | |
| | dihydrobrassicasterol) | | | | |
| 4 | 5,22-cholestadien-24- | 57.7 | 59.2 | 46.8 | 49.9 |
| | ethyl-3-ol (Stigmasterol | (8.4%) | (8.2%) | (7.9%) | (8.3%) |
| | or poriferasterol) | | | | |
| 5 | 24-ethylcholest-5-en- | 9.64 | 9.92 | 9.26 | 10.2 |
| | 3-ol (β-Sitosterol or | (1.4%) | (1.4%) | (1.6%) | (1.7%) |
| | clionasterol) | | | | |
| 6 | Other sterols | 209 | 221 | 216 | 213 |
| | Total sterols | 685.64 | 718.82 | 589.96 | 601.1 |

These results show three striking features. First, ergosterol was found to be the most abundant of all the sterols, accounting for about 50% or more of the total sterols. The amount of ergosterol is greater than that of campesterol, β-sitosterol, and stigmasterol combined. Ergosterol is steroid commonly found in fungus and not commonly found in plants, and its presence particularly in significant amounts serves as a useful marker for non-plant oils. Secondly, the oil was found to contain brassicasterol. With the exception of rapeseed oil, brassicasterol is not commonly found in plant based oils. Thirdly, less than 2% β-sitosterol was found to be present. β-sitosterol is a prominent plant sterol not commonly found in microalgae, and its presence particularly in significant amounts serves as a useful marker for oils of plant origin. In summary, Prototheca moriformis strain UTEX1435 has been found to contain both significant amounts of ergosterol and only trace amounts of β-sitosterol as a percentage of total sterol content. Accordingly, the ratio of ergosterol:β-sitosterol or in combination with the presence of brassicasterol can be used to distinguish this oil from plant oils.

In some embodiments, the oil content of an oil provided herein contains, as a percentage of total sterols, less than 20%, 15%, 10%, 5%, 4%, 3%, 2%, or 1% β-sitosterol. In other embodiments the oil is free from β-sitosterol.

In some embodiments, the oil is free from one or more of β-sitosterol, campesterol, or stigmasterol. In some embodiments the oil is free from β-sitosterol, campesterol, and stigmasterol. In some embodiments the oil is free from campesterol. In some embodiments the oil is free from stigmasterol.

In some embodiments, the oil content of an oil provided herein comprises, as a percentage of total sterols, less than 20%, 15%, 10%, 5%, 4%, 3%, 2%, or 1% 24-ethylcholest-5-en-3-ol. In some embodiments, the 24-ethylcholest-5-en-3-ol is clionasterol. In some embodiments, the oil content of an oil provided herein comprises, as a percentage of total sterols, at least 1%, 2%, 3%, 4%, 5%, 6%, 7%, 8%, 9%, or 10% clionasterol.

In some embodiments, the oil content of an oil provided herein contains, as a percentage of total sterols, less than 20%, 15%, 10%, 5%, 4%, 3%, 2%, or 1% 24-methylcholest-5-en-3-ol. In some embodiments, the 24-methylcholest-5-en-3-ol is 22, 23-dihydrobrassicasterol. In some embodiments, the oil content of an oil provided herein comprises, as a percentage of total sterols, at least 1%, 2%, 3%, 4%, 5%, 6%, 7%, 8%, 9%, or 10% 22,23-dihydrobrassicasterol.

In some embodiments, the oil content of an oil provided herein contains, as a percentage of total sterols, less than 20%, 15%, 10%, 5%, 4%, 3%, 2%, or 1% 5,22-cholestadien-24-ethyl-3-ol. In some embodiments, the 5, 22-cholestadien-24-ethyl-3-ol is poriferasterol. In some embodiments, the oil content of an oil provided herein comprises, as a percentage of total sterols, at least 1%, 2%, 3%, 4%, 5%, 6%, 7%, 8%, 9%, or 10% poriferasterol.

In some embodiments, the oil content of an oil provided herein contains ergosterol or brassicasterol or a combination of the two. In some embodiments, the oil content contains, as a percentage of total sterols, at least 5%, 10%, 20%, 25%, 35%, 40%, 45%, 50%, 55%, 60%, or 65% ergosterol. In some embodiments, the oil content contains, as a percentage of total sterols, at least 25% ergosterol. In some embodiments, the oil content contains, as a percentage of total sterols, at least 40% ergosterol. In some embodiments, the oil content contains, as a percentage of total sterols, at least 5%, 10%, 20%, 25%, 35%, 40%, 45%, 50%, 55%, 60%, or 65% of a combination of ergosterol and brassicasterol.

In some embodiments, the oil content contains, as a percentage of total sterols, at least 1%, 2%, 3%, 4%, or 5% brassicasterol. In some embodiments, the oil content contains, as a percentage of total sterols less than 10%, 9%, 8%, 7%, 6%, or 5% brassicasterol.

In some embodiments the ratio of ergosterol to brassicasterol is at least 5:1, 10:1, 15:1, or 20:1.

In some embodiments, the oil content contains, as a percentage of total sterols, at least 5%, 10%, 20%, 25%, 35%, 40%, 45%, 50%, 55%, 60%, or 65% ergosterol and less than 20%, 15%, 10%, 5%, 4%, 3%, 2%, or 1% β-sitosterol. In some embodiments, the oil content contains, as a percentage of total sterols, at least 25% ergosterol and less than 5% β-sitosterol. In some embodiments, the oil content further comprises brassicasterol.

Sterols contain from 27 to 29 carbon atoms (C27 to C29) and are found in all eukaryotes. Animals exclusively make C27 sterols as they lack the ability to further modify the C27 sterols to produce C28 and C29 sterols. Plants however are able to synthesize C28 and C29 sterols, and C28/C29 plant sterols are often referred to as phytosterols. The sterol profile of a given plant is high in C29 sterols, and the primary sterols in plants are typically the C29 sterols b-sitosterol and stigmasterol. In contrast, the sterol profiles of non-plant organisms contain greater percentages of C27 and C28 sterols. For example the sterols in fungi and in many microalgae are principally C28 sterols. The sterol profile and particularly the striking predominance of C29 sterols over C28 sterols in plants has been exploited for determining the proportion of plant and marine matter in soil samples (Huang, Wen-Yen, Meinschein W. G., “Sterols as ecological indicators”; Geochimica et Cosmochimia Acta. Vol 43. pp 739-745).

In some embodiments the primary sterols in the microalgal oils provided herein are sterols other than b-sitosterol and stigmasterol. In some embodiments of the microalgal oils, C29 sterols make up less than 50%, 40%, 30%, 20%, 10%, or 5% by weight of the total sterol content.

In some embodiments the microalgal oils provided herein contain C28 sterols in excess of C29 sterols. In some embodiments of the microalgal oils, C28 sterols make up greater than 50%, 60%, 70%, 80%, 90%, or 95% by weight of the total sterol content. In some embodiments the C28 sterol is ergosterol. In some embodiments the C28 sterol is brassicasterol.

Where a fatty acid profile of a triglyceride (also referred to as a “triacylglyceride” or “TAG”) cell oil is given here, it will be understood that this refers to a nonfractionated sample of the storage oil extracted from the cell analyzed under conditions in which phospholipids have been removed or with an analysis method that is substantially insensitive to the fatty acids of the phospholipids (e.g. using chromatography and mass spectrometry). The oil may be subjected to an RBD process to remove phospholipids, free fatty acids and odors yet have only minor or negligible changes to the fatty acid profile of the triglycerides in the oil. Because the cells are oleaginous, in some cases the storage oil will constitute the bulk of all the TAGs in the cell. Examples 1 and 2 below give analytical methods for determining TAG fatty acid composition and regiospecific structure.

Broadly categorized, certain embodiments of the invention include (i) recombinant oleaginous cells that comprise an ablation of one or two or all alleles of an endogenous polynucleotide, including polynucleotides encoding lysophosphatidic acid acyltransferase (LPAAT) or (ii) cells that produce oils having low concentrations of polyunsaturated fatty acids, including cells that are auxotrophic for unsaturated fatty acids; (iii) cells producing oils having high concentrations of particular fatty acids due to expression of one or more exogenous genes encoding enzymes that transfer fatty acids to glycerol or a glycerol ester; (iv) cells producing regiospecific oils, (v) genetic constructs or cells encoding a an LPAAT, a lysophosphatidylcholine acyltransferase (LPCAT), a phosphatidylcholine diacylglycerol cholinephosphotransferase (PDCT), diacylglycerol cholinephosphotransferase (DAG-CPT) or fatty acyl elongase (FAE), (vi) cells producing low levels of saturated fatty acids and/or high levels of C18:1, C18:2, C18:3, C20:1 or C22:1, (vii) and other inventions related to producing cell oils with altered profiles. The embodiments also encompass the oils made by such cells, the residual biomass from such cells after oil extraction, oleochemicals, fuels and food products made from the oils and methods of cultivating the cells.

In any of the embodiments below, the cells used are optionally cells having a type II fatty acid biosynthetic pathway such as plant cells, yeast cells, microalgal cells including heterotrophic or obligate heterotrophic microalgal cells, including cells classified as Chlorophyta, Trebouxiophyceae, Chlorellales, Chlorellaceae, or Chlorophyceae, or cells engineered to have a type II fatty acid biosynthetic pathway using the tools of synthetic biology (i.e., transplanting the genetic machinery for a type II fatty acid biosynthesis into an organism lacking such a pathway). Use of a host cell with a type II pathway avoids the potential for non-interaction between an exogenous acyl-ACP thioesterase or other ACP-binding enzyme and the multienzyme complex of type I cellular machinery. In specific embodiments, the cell is of the species Prototheca moriformis, Prototheca krugani, Prototheca stagnora or Prototheca zopfii or has a 23S rRNA sequence with at least 65, 70, 75, 80, 85, 90 or 95% nucleotide identity SEQ ID NO: 25. By cultivating in the dark or using an obligate heterotroph, the cell oil produced can be low in chlorophyll or other colorants. For example, the cell oil can have less than 100, 50, 10, 5, 1, 0.0.5 ppm of chlorophyll without substantial purification.

The stable carbon isotope value δ13C is an expression of the ratio of ¹³C/¹²C relative to a standard (e.g. PDB, carbonite of fossil skeleton of Belemnite americana from Peedee formation of South Carolina). The stable carbon isotope value δ13C (‰) of the oils can be related to the δ13C value of the feedstock used. In some embodiments the oils are derived from oleaginous organisms heterotrophically grown on sugar derived from a C4 plant such as corn or sugarcane. In some embodiments the δ13C (‰) of the oil is from −10 to −17‰ or from −13 to −16‰.

In specific embodiments and examples discussed below, one or more fatty acid synthesis genes (e.g., encoding an acyl-ACP thioesterase, a keto-acyl ACP synthase, an LPAAT, an LPCAT, a PDCT, a DAG-CPT, an FAE a stearoyl ACP desaturase, or others described herein) is incorporated into a microalga. It has been found that for certain microalga, a plant fatty acid synthesis gene product is functional in the absence of the corresponding plant acyl carrier protein (ACP), even when the gene product is an enzyme, such as an acyl-ACP thioesterase, that requires binding of ACP to function. Thus, optionally, the microalgal cells can utilize such genes to make a desired oil without co-expression of the plant ACP gene.

For the various embodiments of recombinant cells comprising exogenous genes or combinations of genes, it is contemplated that substitution of those genes with genes having 60%, 70%, 80%, 85%, 90%, 91%, 92%, 93%, 94%, 95%, 96%, 97%, 98%, or 99% or 100% nucleic acid sequence identity can give similar results, as can substitution of genes encoding proteins having 60%, 70%, 80%, 85%, 90%, 91% 92%, 93%, 94%, 95%, 95.5%, 96%, 96.5%, 97%, 97.5%, 98%, 98.5%, 99% or 100% amino acid sequence identity. Nucleic acids encoding the acyltransferases encode acyltransferases that have 60%, 65%, 70%, 75%, 80%, 85%, 90%, 95%, 98%, 99%, or 100%, or at least 60%, 65%, 70%, 75%, 80%, 85%, 90%, 95%, 98%, 99%, or 100% amino acid sequence identity to the acyltransferase disclosed in clade 1, clade 2, clade 3 or clade 4 of Table 5. Likewise, for novel regulatory elements, it is contemplated that substitution of those nucleic acids with nucleic acids having 60%, 70%, 80%, 85%, 90%, 91%, 92%, 93%, 94%, 95%, 96%, 97%, 98%, 99% or 100% nucleic acid can be efficacious. In the various embodiments, it will be understood that sequences that are not necessary for function (e.g. FLAG® tags or inserted restriction sites) can often be omitted in use or ignored in comparing genes, proteins and variants.

The novel genes and gene combinations reported here can be used in higher plants using techniques that are well known in the art. For example, the use of exogenous lipid metabolism genes in higher plants is described in U.S. Pat. Nos. 6,028,247; 5,850,022; 5,639,790; 5,455,167; 5,512,482; and 5,298,421 disclose higher plants with exogenous acyl-ACP thioesterases. WO2009129582 and WO1995027791 disclose cloning of LPAAT in plants. FAD2 ablation and/or down regulation in higher plants is taught in WO 2013112578, and WO2008/006171. SAD ablation and/or down regulation in higher plants is taught in WO 2013112578, and WO 2008006171.

The expression of the novel acyltransferases is shown in Examples 4, 5, 6 and 7. The expression of Cuphea paucipetala or Cuphea ignea LPATs markedly increased the C8:0 and C10:0 fraction of the cell oil. Additionally, the expression of Cuphea paucipetala or Cuphea ignea LPAATs markedly increased the incorporation of C8:0 and C10:0 fatty acids in the sn-2 position of the TAG. This is disclosed in Example 4.

The expression of LPAT genes in host cells increased C18:2 levels and elevated the sat-unsat-sat/sat-sat-sat, (e.g., SOS/SSS) ratio of the cell oil. For example, the expression of Theobroma cacoa LPAT2 drives the transfer of unsaturated fatty acids toward the sn-2 position and reduces the incorporation of saturated fatty acids at sn-2.

The novel LPAATs, GPATs, DGATs, LPCATs, and PLA2 with specificity for mid-chain fatty acids are disclosed. In Example 7, expression of LPAATs and DGATs are disclosed.

When an acyltransferase of the invention is expressed in a host cell, one or more additional exogenous genes can concomitantly be expressed. An embodiment of this invention provides host cells that express a recombinant acyltransferase and concomitantly express one or more additional recombinant genes. The one or more additional genes include invertase, fatty acyl-ACP thioesterase (FATA, FATB), melibiase, ketoacyl synthase (KASI, KASII, KASIII, KASIV), antibiotic selective markers, tags such as FLAG, and THIC. In Examples 4, 5, 6, and 7, the co-expression of nucleic acids that encode LPAATs co-expressed with one or more exogenous genes that encode invertase, fatty acyl-ACP thioesterase, melibiase, ketoacyl synthase, THIC are disclosed.

When an acyltransferase of the invention is expressed in a host cell, an endogenous gene of the host call can concomitantly be ablated or downregulated, thereby eliminating or decreasing the expression of the gene of the host cell. This can be accomplished by using homologous recombination techniques or other RNA inhibitory technologies. The ablated or downregulated gene can be any gene in the host cell. The ablated or downregulated endogenous gene can be stearoyl ACP desaturase, fatty acyl desaturase, fatty acyl-ACP thioesterase (FATA or FATB), ketoacyl synthase (KASI, KASII, KASIII or KAS IV), or an acyltransferase (LPAAT, DGAT, GPAT, LPCAT). When an endogenous is ablated, one, two or more alleles of the endogenous can be ablated. In Example 5, the expression of a Brassica LPAAT, while concomitantly ablating an endogenous stearoyl ACP desaturase is disclosed. In Example 6, LPAATs, GPATs, DGATs, LPCATs and PLA2s with specificity for mid-chain fatty acids were expressed, while ablating a gene encoding stearoyl ACP desaturase. In Example 7 the down regulation of an endogenous FAD2 and a hairpin RNA is disclosed. In co-owned PCT/US2016/026265, applicants disclosed concomitant ablation of an endogenous LPAAT and expression of an exogenous LPAAT.

In one embodiment, the expression of the acyl transferases alters the fatty acid profile and/or the sn-2 profile of the oil produced by the host organism. The fatty acid profiles and the sn-2 profiles that result from the expression of various acyltransferases are disclosed in Tables 6, 7, 10, 11, 12, 13, 16, 17, 18, 19, 20, 22, 23, and 24. The invention provides host cells with altered fatty acid profiles and altered sn-2 profiles according to Tables 6, 7, 10, 11, 12, 13, 16, 17, 18, 19, 20, 22, 23, and 24.

As described in PCT/US2016/026265, co-owned by applicant, transcript profiling was used to discover promoters that modulate expression in response to low nitrogen conditions. The promoters are useful to selectively express various genes and to alter the fatty acid composition of microbial oils. In accordance with an embodiment, there are non-natural constructs comprising a heterologous promoter and a gene, wherein the promoter comprises at least 60%, 65%, 70%, 75%, 80%, 85%, 90%, 95%, or 100% sequence identity to any of the promoters of SEQ ID NOs: 1-18 and the gene is differentially expressed under low vs. high nitrogen conditions. In particular, the Prototheca moriformis AMT02 (SEQ ID NO: 18) and AMT03 promoter (SEQ ID NO: 18) are useful promoters for controlling the expression of an exogenous gene. For example, the promoters can be placed in front of a FAD2 gene in a linoleic acid auxotroph to produce an oil with less than 5, 4, 3, 2, or 1% linoleic acid after culturing first under high nitrogen conditions, then next culturing under low nitrogen conditions. Additional promoters, in particulare Prototheca and Chlorella promoters are described in the sequences and descriptions in this application. For example, the Prototheca HXT1, SAD, LDH1 and other Prototheca promoters are described in Examples 6, 7, 8, and 9. Additionally, the Chlorella SAD, ACT and other Chlorella promoters are described in Examples 6, 7, 8, and 9.

In embodiments of the present invention, oleaginous cells expressing one or more of the genes encoding acyltransferases and/or variant FATA can produce an oil with at least 20, 40, 60 or 70% of C8, C10, C12, C14, C16, or C18 fatty acids.

The invention also provides host cells expressing one or more of the genes encoding acyltransferases and/or variant FATA can produce an oil enriched is oils that are sat-unsat-sat. Oils of this type include SOS, POP, POS, SLS, PLO, PLO. The sat-unsat-sat oils comprise at least 30%, 40%, 50%, 60%, 65%, 70%, 75%, 80%, 85%, 90%, or 95% of the cell oil by dry cell weight.

The invention also provides host cells expressing one or more of the genes encoding acyltransferases and/or variant FATA can produce an oil that is decreased in tri-saturated oils, sat-sat-sat. Oils of this type include PPP, PSS, PPS, SSS, SPS, and PSP. The sat-sat-sat oils comprise less than 50%, 40%, 30%, 20%, 15%, 10%, 8%, 6%, 5%, 4%, 3%, 2%, or 1% of the cell oil by molar fraction or dry cell weight.

The host cells of the invention can produce 25%, 30%, 40%, 50%, 60%, 70%, 80%, 85%, or about 90% oil by cell weight, ±5%. Optionally, the oils produced can be low in DHA or EPA fatty acids. For example, the oils can comprise less than 5%, 2%, or 1% DHA and/or EPA.

In other embodiments of the invention, there is a process for producing an oil, triglyceride, fatty acid, or derivative of any of these, comprising transforming a cell with any of the nucleic acids discussed herein. In another embodiment, the transformed cell is cultivated to produce an oil and, optionally, the oil is extracted. Oil extracted in this way can be used to produce food, oleochemicals or other products.

The oils discussed above alone or in combination are useful in the production of foods, fuels and chemicals (including plastics, foams, films, etc). The oils, triglycerides, fatty acids from the oils may be subjected to C—H activation, hydroamino methylation, methoxy-carbonation, ozonolysis, enzymatic transformations, epoxidation, methylation, dimerization, thiolation, metathesis, hydro-alkylation, lactonization, or other chemical processes.

After extracting the oil, a residual biomass may be left, which may have use as a fuel, as an animal feed, or as an ingredient in paper, plastic, or other product. For example, residual biomass from heterotrophic algae can be used in such products.

### EXAMPLES

### Example 1: Fatty Acid Analysis by Fatty Acid Methyl Ester Detection

Lipid samples were prepared from dried biomass. 20-40 mg of dried biomass was resuspended in 2 mL of 5% H₂SO₄ in MeOH, and 200 ul of toluene containing an appropriate amount of a suitable internal standard (C19:0) was added. The mixture was sonicated briefly to disperse the biomass, then heated at 70-75° C. for 3.5 hours. 2 mL of heptane was added to extract the fatty acid methyl esters, followed by addition of 2 mL of 6% K₂CO₃ (aq) to neutralize the acid. The mixture was agitated vigorously, and a portion of the upper layer was transferred to a vial containing Na₂SO₄ (anhydrous) for gas chromatography analysis using standard FAME GC/FID (fatty acid methyl ester gas chromatography flame ionization detection) methods. Fatty acid profiles reported below were determined by this method.

### Example 2: Analysis of Regiospecific Profile

LC/MS TAG distribution analyses were carried out using a Shimadzu Nexera ultra high performance liquid chromatography system that included a SIL-30AC autosampler, two LC-30AD pumps, a DGU-20A5 in-line degasser, and a CTO-20A column oven, coupled to a Shimadzu LCMS 8030 triple quadrupole mass spectrometer equipped with an APCI source. Data was acquired using a Q3 scan of m/z 350-1050 at a scan speed of 1428 u/sec in positive ion mode with the CID gas (argon) pressure set to 230 kPa. The APCI, desolvation line, and heat block temperatures were set to 300, 250, and 200° C., respectively, the flow rates of the nebulizing and drying gases were 3.0 L/min and 5.0 L/min, respectively, and the interface voltage was 4500 V. Oil samples were dissolved in dichloromethane-methanol (1:1) to a concentration of 5 mg/mL, and 0.8 μL of sample was injected onto Shimadzu Shim-pack XR-ODS III (2.2 μm, 2.0×200 mm) maintained at 30° C. A linear gradient from 30% dichloromethane-2-propanol (1:1)/acetonitrile to 51% dichloromethane-2-propanol (1:1)/acetonitrile over 27 minutes at 0.48 mL/min was used for chromatographic separations.

### Example 3: Cultivation of Microalgae

### Standard Lipid Production Conditions:

Cells scraped from a source plate with toothpicks were used to inoculate pre-seed cultures of 0.5 mL EB03, 0.5% glucose, 1×DAS2 cultures in 96-well blocks. Pre-seed cultures were grown for 70-75 h at 28° C., 900 rpm in a Multitron shaker. 40 μl of pre-seed cultures were used to inoculate seed cultures of 0.46 mL H29, 4% glucose, 25 mM citrate pH 5 or 100 mM PIPES pH 7.3, 1×DAS2 (8% inoculum), and grown for 24-28 h at 28° C., 900 rpm in a Multitron shaker. 40 μL of seed cultures were used to inoculate lipid production cultures of 0.46 mL H43, 6% glucose, 25 mM citrate pH 5, 1×DAS2 (8% inoculum), and grown for 70-75 h at 28° C., 900 rpm in a Multitron shaker. Fatty acid profiles and lipid titer analyses were performed as disclosed in Examples 1 and 2.

### 50 mL Shake Flask Format

Cells scraped from a source plate with inoculation loops, or cell cultures from cryovials were used to inoculate pre-seed cultures of 10 mL EB03, 0.5% glucose, 1×DAS2 cultures in 50 mL bioreactor tubes. Pre-seed cultures were grown for 70-75 h at 28° C., 200 rpm in a Kuhner shaker. 0.8 mL of pre-seed cultures were used to inoculate seed cultures of 10 mL H29, 4% glucose, 25 mM citrate pH 5 or 100 mM PIPES pH 7.3, 1×DAS2 (8% inoculum), and grown for 24-28 h at 28° C., 200 rpm in a Kuhner shaker. 100 μL of seed cultures were used to inoculate lipid production cultures of 49.9 mL H43, 6% glucose, 25 mM citrate pH 5 or 100 mM PIPES pH 7.3, 1×DAS2 (0.2% inoculum), and grown for 118-122 h at 28° C., 200 rpm in a Kuhner shaker. Fatty acid profiles and lipid titer analyses were performed as disclosed in Examples 1 and 2.

### EB03

| Dry chemicals | |
|---|---|
| Component | Concentration (g/L) |
| K2HPO4 | 3 |
| Sodium Phosphate Dibasic Heptahydrate | 5.66 |
| (Na2HPO4 7H2O) | |
| citric acid monohydrate | 1.2 |
| ammonium sulfate | 1 |
| MgSO4 7H2O | 0.23 |
| CaCl2 2H2O | 0.03 |

| Stock solutions | |
|---|---|
| Component | Concentration (mL/L) |
| 100X C-Trace (3) | 10 |
| Antifoam Sigma 204 | 0.225 |

### H29

| Dry chemicals | Final |
|---|---|
| Component | Concentration (g/L) |
| K₂HPO₄ (Potassium phosphate | 0.25 |
| dibasic anhydrous) | |
| NaH₂PO₄ (Sodium phosphate | 0.18 |
| monobasic) | |
| MgSO₄•7H₂O (Magnesium | 0.24 |
| sulfate heptahydrate) | |
| Citric acid monohydrate | 0.25 |
| Stock solutions | |
| Component | Concentration (mL/L) |
| 0.017M stock CaCl₂•2H₂O | 10 |
| 0.151M (NH₄)₂SO₄ | 52.2 |
| 100X C-Trace (2) | 10 |
| Antifoam Sigma 204 | 0.225 |

### H43

| Dry chemicals | Final |
|---|---|
| Component | Concentration (g/L) |
| K2HPO4 | 0.25 |
| NaH2PO4 | 0.18 |
| MgSO4 7H2O | 0.24 |
| Citric acid H2O | 0.25 |
| Stock solutions | |
| Component | Concentration (mL/L) |
| 0.017M stock CaCl2 2H2O | 10 |
| 100X C-Trace (2) | 10 |
| Antifoam Sigma 204 | 0.225 |
| 0.151M (NH4)2SO4 | 12.5 |

### 1000×DAS2

| Dry chemicals | Final |
|---|---|
| Component | Concentration (g/L) |
| Thiamine-HCl | 0.67 |
| d-Biotin | 0.010 |
| Cyanocobalimin (vit B-12) | 0.008 |
| Calcium Pantothenate | 0.02 |
| PABA (p-aminobenzoic acid) | 0.04 |

### 100×C-Trace(2)

| Dry chemicals | Final |
|---|---|
| Component | Concentration (g/L) |
| CuSO4—5H2O | 0.011 |
| CoC12—6H2O | 0.081 |
| H3BO3 | 0.33 |
| ZnSO4—7H2O | 1.4 |
| MnSO4—H2O | 0.81 |
| Na2MoO4—2H2O | 0.039 |
| FeSO4—7H2O | 0.11 |
| NiCl2—6H2O | 0.013 |
| Citric Acid Monohydrate | 3.0 |

### 100×C-Trace (3)

| Dry chemicals | Final |
|---|---|
| Component | Concentration (g/L) |
| CuSO4—5H2O | 0.011 |
| H3BO3 | 0.33 |
| ZnSO4—7H2O | 1.4 |
| MnSO4—H2O | 0.81 |
| Na2MoO4—2H2O | 0.039 |
| FeSO4—7H2O | 0.11 |
| NiCl2—6H2O | 0.013 |
| Citric Acid Monohydrate | 3.0 |

### Example 4: Identification of Novel LPAAT Genes from Sequenced Transcriptomes and Engineering Sn-2 Tag Regiospecificity in UTEX1435 by Expression of Heterologous LPAAT Genes from Cuphea paucipetala, Cuphea ignea, Cuphea painteri, and Cuphea hookeriana

Lysophosphatidic acyltransferase (LPAAT) genes from plant seeds were cloned and expressed in the transgenic strain, S6511, derived from UTEX 1435 (P. moriformis). Expression of the heterologous LPAATs increases C8:0 and C10:0 fatty acid levels and dramatically increases incorporation of C8:0 and C10:0 fatty acids at the sn-2 position of triacylglycerols (TAGs) in transgenic strains.

TAGs are synthesized from various chain length acyl-CoAs and glycerol-3-phosphate by consecutive action of three ER-resident enzymes of the Kennedy pathway-glycerol phosphate acyltransferase (GPAT), LPAAT, and diacylglycerol acyltransferase (DGAT). Substrate specificities of these acyltransferases are known to determine the fatty acid composition of the resulting TAGs. LPAAT acylates the sn-2 hydroxyl group of lysophosphatidic acid (LPA) to form phosphatidic acid (PA), a precursor to TAG. In co-owned applications WO2013/158938, WO2015/051139, and PCT/US2016/026265 we demonstrated expression of LPAAT from Cocos nucifera (CnLPAAT, accession no. AAC49119; Knutzon et al., 1995).

Strain S6511 expresses the acyl-ACP thioesterase (FATB2) gene from Cuphea hookeriana (ChFATB2), leading to C8:0 and C10:0 fatty acid accumulation of ca. 14% and 28%, respectively. Strain S6511 is a strain made according to the methods disclosed in co-owned WO2010/063031 and WO2010/063032, herein incorporated by reference. Briefly, S6511 is a strain that express sucrose invertase and a C. hookeriana FATB2. The construct pSZ3101: 6S::CrTUB2-ScSUC2-CvNR_a:PmAMT03-CpSAD1tp_trimmed:ChFATB2-CvNR_d::6S was engineered into S3150, a strain classically mutagenized to increase lipid yield. We identified novel C8:0- and C10:0-specific LPAATs from seeds exhibiting high levels of C8:0 and C10:0 fatty acids. After we identified and cloned LPAATs we expressed the LPAAT genes in S6511.

### Method for Identification of LPAATs

Seeds were obtained from species exhibiting elevated levels of midchain and other specialized fatty acids (Table 4).

TABLE 4
Fatty acid profiles of mature seeds.

| | | | | | | | | | C18:1 | | | | | | | C22: | |
|---|---|---|---|---|---|---|---|---|---|---|---|---|---|---|---|---|---|
| | | C8: | C10: | C12: | C14: | C16: | C18: | C18: | (petro- | C18: | C20: | C20: | C22: | C22: | C22: | 2n9, | C22: |
| | | 0 | 0 | 0 | 0 | 0 | 0 | 1 | selinate) | 2 | 0 | 1 | 0 | 1n17 | 1n9 | 17 | 2n6 |
| S01_Cc | Cinnamomum | 0.4 | 54.7 | 39.0 | 1.6 | 0.7 | 0.1 | 2.9 | | 0.6 | 0.0 | | | | | | |
| | camphora | | | | | | | | | | | | | | | | |
| S02_Uc | Umbellularia | 0.9 | 28.8 | 63.0 | 2.3 | 0.4 | 0.1 | 3.4 | | 0.6 | 0.0 | | | | | | |
| | californica | | | | | | | | | | | | | | | | |
| S03_Ld | Limnanthes | 0.0 | 0.0 | 0.0 | 0.4 | 0.7 | 0.4 | 2.7 | | 1.5 | 1.5 | 59.9 | 0.3 | 2.8 | 17.4 | 9.3 | 0.5 |
| | douglasii | | | | | | | | | | | | | | | | |
| S04_Chs | Cuphea | 0.2 | 6.5 | 83.7 | 5.1 | 1.1 | 0.1 | 0.0 | | 1.7 | 0.1 | | | | | | |
| | hyssopifolia | | | | | | | | | | | | | | | | |
| S05_Ccr | Cuphea | 1.6 | 8.1 | 59.2 | 15.2 | 3.9 | 0.6 | 0.0 | | 5.4 | 0.2 | | | | | | |
| | carthagenensis | | | | | | | | | | | | | | | | |
| S06_Cpr | Cuphea | 2.0 | 11.5 | 61.3 | 10.8 | 2.7 | 0.5 | 0.0 | | 5.2 | 0.1 | | | | | | |
| | parsonsia | | | | | | | | | | | | | | | | |
| S07_Cg | Cuphia | 7.1 | 85.1 | 1.7 | 0.3 | 1.0 | 0.2 | 0.0 | | 2.1 | 0.1 | | | | | | |
| | glossostoma | | | | | | | | | | | | | | | | |
| S08_Cht | Cuphea | 3.5 | 44.3 | 40.0 | 4.3 | 1.2 | 0.3 | 2.2 | | 3.6 | 0.1 | | | | | | |
| | heterophylla | | | | | | | | | | | | | | | | |
| S11_Dc | Daucus | 0.0 | 0.0 | 0.0 | 0.1 | 5.9 | 0.8 | 11.5 | 65.9 | 13.0 | 0.5 | 0.3 | 0.3 | | | | |
| | carrota | | | | | | | | | | | | | | | | |
| S14_Cw | Cuphea | 0.5 | 20.2 | 62.5 | 5.8 | 2.2 | 0.3 | 2.7 | | 4.7 | | | | | | | |
| | wrightii | | | | | | | | | | | | | | | | |
| S15_Bj | Brassica | 0.0 | 0.0 | 0.0 | 0.1 | 3.2 | 0.7 | 12.1 | | 19.2 | 0.5 | 6.3 | 0.8 | | 38.9 | | 1.3 |
| | juncea | | | | | | | | | | | | | | | | |
| S16_Br | Brassica | 0.0 | 0.0 | 0.0 | 0.1 | 2.8 | 1.0 | 16.0 | | 16.8 | 0.7 | 8.3 | 1.0 | | 40.4 | | 0.8 |
| | rapa | | | | | | | | | | | | | | | | |
| | nipposinica | | | | | | | | | | | | | | | | |
| S17_Ca | Cuphea | 90.8 | 2.7 | 0.0 | 0.1 | 1.2 | 0.1 | 1.8 | | 2.8 | | | | | | | |
| | avigera var. | | | | | | | | | | | | | | | | |
| | pulcherrima | | | | | | | | | | | | | | | | |
| S18_Ch | Cuphea | 64.7 | 29.7 | 0.1 | 0.2 | 1.3 | 0.1 | 1.9 | | 2.0 | | | | | | | |
| | hookeriana | | | | | | | | | | | | | | | | |
| S19_Cpal | Cuphea | 28.9 | 0.8 | 1.3 | 55.1 | 6.2 | 0.2 | 3.0 | | 3.4 | | | | | | | |
| | palustris | | | | | | | | | | | | | | | | |
| S20_Cpai | Cuphea | 67.0 | 20.8 | 0.1 | 0.2 | 2.6 | 0.3 | 3.1 | | 4.5 | | | | | | | |
| | painteri | | | | | | | | | | | | | | | | |
| S21_Cpau | Cuphea | 1.5 | 91.0 | 1.2 | 0.7 | 1.5 | 0.2 | 1.1 | | 2.1 | | | | | | | |
| | paucipetala | | | | | | | | | | | | | | | | |
| S22_Chook | Cuphea | 62.8 | 31.9 | 0.2 | 0.2 | 1.0 | 0.1 | 2.1 | | 1.2 | | | | | | | |
| | hookeriana | | | | | | | | | | | | | | | | |
| S23_Cglut | Cuphea | 5.2 | 29.9 | 46.4 | 3.9 | 1.9 | 0.4 | 0.0 | | 8.1 | | | | | | | |
| | glutinosa | | | | | | | | | | | | | | | | |
| S24_Caequ | Cuphea | 27.1 | 0.0 | 1.4 | 57.4 | 6.0 | 0.2 | 3.2 | | 3.8 | | | | | | | |
| | aequipetala | | | | | | | | | | | | | | | | |
| S25_Ccalc | Cuphea | 8.0 | 20.4 | 46.8 | 7.6 | 3.2 | 0.6 | 3.7 | | 8.5 | | | | | | | |
| | calcarata | | | | | | | | | | | | | | | | |
| S26_Chook | Cuphea | 70.4 | 23.1 | 0.1 | 0.2 | 1.5 | 0.2 | 2.5 | | 1.8 | | | | | | | |
| | hookeriana | | | | | | | | | | | | | | | | |
| S27_Cproc | Cuphea | 0.9 | 86.3 | 0.0 | 1.6 | 2.2 | 0.4 | 3.2 | | 3.3 | | | | | | | |
| | procumbens | | | | | | | | | | | | | | | | |
| S28_Cignea | Cuphea | 3.1 | 84.9 | 0.7 | 0.3 | 2.6 | 0.2 | 2.9 | | 4.4 | | | | | | | |
| | ignea | | | | | | | | | | | | | | | | |
| S35_Ccras | Cuphea | 1.3 | 87.7 | 1.3 | 0.4 | 2.0 | 0.5 | 3.3 | | 2.7 | | | | | | | |
| | crassiflora | | | | | | | | | | | | | | | | |
| S36_Ckoe | Cuphea | 0.0 | 87.4 | 1.4 | 0.8 | 2.2 | 0.4 | 2.3 | | 4.5 | | | | | | | |
| | koehneana | | | | | | | | | | | | | | | | |
| S37_Clept | Cuphea | 1.3 | 86.1 | 1.3 | 0.4 | 2.2 | 0.5 | 3.1 | | 4.1 | | | | | | | |
| | leptopoda | | | | | | | | | | | | | | | | |
| S40_Clop | Cuphea | 0.5 | 82.3 | 2.4 | 1.6 | 3.0 | 0.6 | 3.9 | | 4.9 | | | | | | | |
| | lophostoma | | | | | | | | | | | | | | | | |
| S41_Sal | Sassafras | 4.3 | 65.2 | 22.8 | 0.9 | 0.8 | 5.1 | 0.0 | | 0.6 | | | | | | | |
| | albidum db | | | | | | | | | | | | | | | | |

The percentage of each fatty acid making up the seed oil is shown; abundant and unusual fatty acid species are indicated in bold.

Briefly, RNA was extracted from dried plant seeds and submitted for paired-end sequencing using the Illumina Hiseq 2000 platform. RNA sequence reads were assembled into corresponding seed transcriptomes using the Trinity software package. LPAAT-containing cDNA contigs were identified by mining transcriptomes for sequences with homology to a known LPAAT that was previously identified in-house, CuPSR23 LPAAT2-1 (see WO2013/158938), using BLAST. For some sequences, a high-confidence, full-length transcript was assembled using Trinity. The resulting amino acid sequences of all new LPAATs were subjected to phylogenetic analyses using previously known, full-length LPAAT sequences (available via NCBI) as well as sequences of previously known LPAATs whose sequences were derived at Solazyme. The analysis showed that the amino acid sequences of the newly discovered LPPAATs were not similar to previously known LPAATs. Table 5 shows the clade analysis in which the novel LPAATs were clustered according to a neighbor joining algorithm. These were found to form 4 clades as listed in Table 5.

TABLE 5
Clade Analysis of LPAATs

| | | | | Percent |
|---|---|---|---|---|
| | | | | amino acid |
| | Amino Acid | | | identity |
| Clade | SEQ ID Nos. | | | to members |
| No. | in Clade | Full Genus Species | Function | of clade |
| 1 | S15 BjLPAAT1d | Brassica juncea | | 96.3 |
| | S15 BjLPAAT1c | Brassica juncea | | |
| | S15 BjLPAAT1a | Brassica juncea | | |
| | S15 BjLPAAT1b | Brassica juncea | | |
| 2 | CuPSR23LPAAT2-1 | Cuphea PSR23 | Prefer C8/ | 93.9 |
| | S40 ClopLPAAT1 | Cuphea lophostoma | C10 sn-2 | |
| | S21 CpauLPAAT1 | Cuphea paucipetala | | |
| | S37 CleptLPAAT1 | Cuphea leptopoda | | |
| | S27 CprocLPAAT1b | Cuphea procumbens | | |
| | S27 CprocLPAAT1 | Cuphea procumbens | | |
| | S04 ChsLPAAT2 | Cuphea hyssopifolia | | |
| | S28 CigneaLPAAT1 | Cuphea ignea | | |
| | S05 CcrLPAAT2a | Cuphea carthagenensis | | |
| | S06 CprLPAAT1 | Cuphea parsonsia | | |
| | S05 CcrLPAAT2b | Cuphea carthagenensis | | |
| | S17 CaLPAAT3 | Cuphea avigera var. | | |
| | | pulcherrima | | |
| | S26 ChookLPAAT1 | Cuphea hookeriana | | |
| | S20 CpaiLPAAT1 | Cuphea painteri | | |
| | S04 ChsLPAAT1 | Cuphea hyssopifolia | | |
| | S25 Ccalc1a | Cuphea calcarata | | |
| | S25 Ccalc1b | Cuphea calcarata | | |
| | S14 CwLPAAT1 | Cuphea wrightii | | |
| | S08 ChtLPAAT1a | Cuphea heterophylla | | |
| | S08 ChtLPAAT1b | Cuphea heterophylla | | |
| | S36 CkoeLPAAT2 | Cuphea koehneana | | |
| | S02 UcLPAAT1b | Umbellularia californica | | |
| | S02 UcLPAAT1a | Umbellularia californica | | |
| | S01 CcLPAAT1a | Cinnamomum camphora | | |
| | S01 CcLPAAT1b | Cinnamomum camphora | | |
| | S41 SaILPAAT1 | Sassafras albidum db | | |
| 3 | S14 CwLPAAT2a | Cuphea wrightii | C18:2 | 86.5 |
| | S14 CwLPAAT2b | Cuphea wrightii | | |
| | S25 CcalcLPAAT2 | Cuphea calcarata | | |
| | S19 CpaILPAAT1 | Cuphea palustris | | |
| | S22 ChookLPAAT3b | Cuphea hookeriana | | |
| | S17 CaLPAAT1 | Cuphea avigera var. | | |
| | | pulcherrima | | |
| | S22 ChookLPAAT3a | Cuphea hookeriana | | |
| | CuPSR23LPAAT3-1 | Cuphea PSR23 | | |
| | S27 CprocLPAAT2b | Cuphea procumbens | | |
| | S27 CprocLPAAT2a | Cuphea procumbens | | |
| | S18 ChLPAAT2a | Cuphea hookeriana | | |
| | S24 CaequLPAAT1d | Cuphea aequipetala | | |
| | S24 CaequLPAAT1b | Cuphea aequipetala | | |
| | S24 CaequLPAAT1a | Cuphea aequipetala | | |
| | S24 CaequLPAAT1c | Cuphea aequipetala | | |
| | S23 CglutLPAAT1a | Cuphea glutinosa | | |
| | S23 CglutLPAAT1b | Cuphea glutinosa | | |
| | S26 ChookLPAAT2b | Cuphea hookeriana | | |
| | S07 CgLPAAT1c | Cuphia glossostoma | | |
| | S07 CgLPAAT1b | Cuphia glossostoma | | |
| | S07 CgLPAAT1a | Cuphia glossostoma | | |
| | S28 CigneaLPAAT2 | Cuphea ignea | | |
| | S36 CkoeLPAAT1 | Cuphea koehneana | | |
| | S35 CcrasLPAAT1a | Cuphea crassiflora | | |
| | S35 CcrasLPAAT1c | Cuphea crassiflora | | |
| | S35 CcrasLPAAT1b | Cuphea crassiflora | | |
| | S35 CcrasLPAAT1d | Cuphea crassiflora | | |
| 4 | Gh LPAAT2B | Garcinia hombroriana | Reduced | 78.5 |
| | Gi LPAAT2B-1 | Garcinia indica | trisaturates, | |
| | Gh LPAAT2A | Garcinia hombroriana | increase | |
| | Gi LPAAT2A | Garcinia indica | unsaturates | |
| | Gh LPAAT2C | Garcinia hombroriana | at Sn-2 | |
| | Gi LPAAT2C-2 | Garcinia indica | position | |
| | S03 LdLPAAT1 | Limnanthes douglasii | | |
| | S11 DcLPAAT1 | Daucus carrota | | |
| | | (carrot) | | |
| | S11 DcLPAAT2 | Daucus carrota | | |
| | | (carrot) | | |
| | S11 DcLPAAT2 | Daucus carrota | | |
| | (truncated) | (carrot) | | |

Functionality of LPAATs in P. moriformis

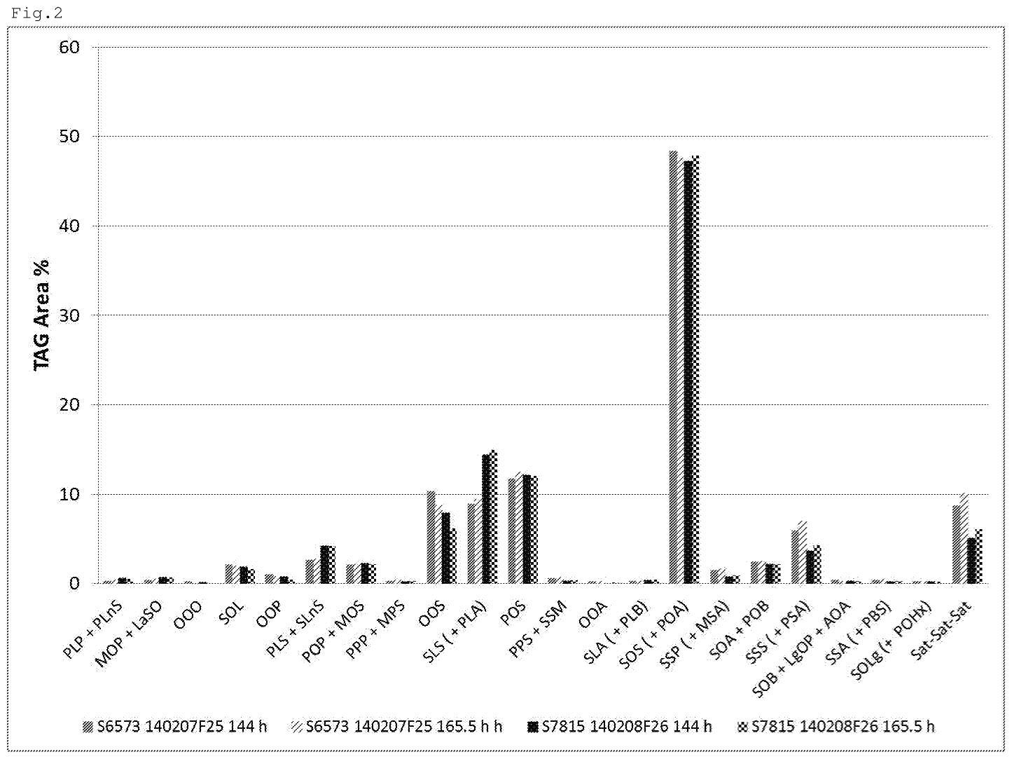

To increase the levels of C8:0 and C10:0 fatty acids in strain S6511, as well as to test the functionality of the newly identified LPAATs, we identified midchain-specific LPAATs from the transcriptomes of species exhibiting high levels of C8:0 and C10:0 fatty acids in their oil seeds and introduced the genes into S6511. LPAATs that co-clustered with CuPSR23 LPAAT2-1, specifically CpauLPAAT1, CigneaLPAAT1, ChookLPAAT1, and CpaiLPAAT1, were selected for synthesis and testing. CpauLPAAT1, CigneaLPAAT1, ChookLPAAT1, and CpaiLPAAT1 were synthesized in a codon-optimized form to reflect UTEX 1435 codon usage. Transgenic strains were generated via transformation of the strain S6511 with a construct encoding one of the four LPAAT genes. The construct pSZ3840 encoding CpauLPAAT1 is shown as an example, but identical methods were used to generate each of the remaining three constructs. Construct pSZ3840 can be written as pLOOP::PmHXT1-ScarMEL1-CvNR:PmAMT3-CpauLPAAT1-CvNR::pLOOP. The sequence of the transforming DNA is provided in FIG. 2 (pSZ3840). The relevant restriction sites in the construct from 5′-3′, BspQI, KpnI, SpeI, XhoI, EcoRI, SpeI, XhoI, SacI, BspQI, respectively, are indicated in lowercase, bold, and underlined. BspQI sites delimit the 5′ and 3′ ends of the transforming DNA. Bold lowercase sequences at the 5′ and 3′ end of the construct represent genomic DNA from UTEX 1435 that target integration to the pLOOP locus via homologous recombination. Proceeding in the 5′ to 3′ direction, the selection cassette has the P. moriformis HXT1 promoter driving expression of the Saccharomyces carlsbergensis MEL1 (conferring the ability to grow on melibiose) and the Chlorella vulgaris Nitrate reductase (NR) gene 3′ UTR. The promoter is indicated by lowercase, boxed text. The initiator ATG and terminator TGA for ScarMEL1 are indicated in bold, uppercase italics, while the coding region is indicated with lowercase italics. The 3′ UTR is indicated by lowercase underlined text. The second cassette containing the codon optimized CpauLPAAT1 gene from Cuphea paucipetala is driven by the P. moriformis AMT3 promoter and has the Chlorella vulgaris Nitrate reductase (NR) gene 3′ UTR. In this cassette, the AMT3 promoter is indicated by lowercase, boxed text. The initiator ATG and terminator TGA for the CpauLPAAT1 gene are indicated in bold, uppercase italics, while the coding region is indicated by lowercase italics. The 3′ UTR is indicated by lowercase underlined text. The final construct was sequenced to ensure correct reading frame and targeting sequences.

SEQ ID NO: 19
pSZ3840/D2554 transforming construct (CpauLPAAT1)

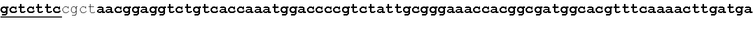

aatacaatattcagtatgtcgcgggcggcgacggcggggagctgatgtcgcgctgggtattgcttaatcgccagcttcgcccccgt
cttggcgcgaggcgtgaacaagccgaccgatgtgcacgagcaaatcctgacactagaagggctgactcgcccggcacggctgaa
ttacacaggcttgcaaaaataccagaatttgcacgcaccgtattcgcggtattttgttggacagtgaatagcgatgcggcaatggc
ttgtggcgttagaaggtgcgacgaaggtggtgccaccactgtgccagccagtcctggcggctcccagggccccgatcaagagcca
ggacatccaaactacccacagcatcaacgccccggcctatactcgaaccccacttgcactctgcaatggtatgggaaccacgggg

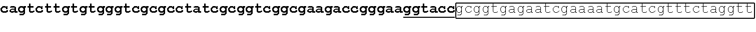

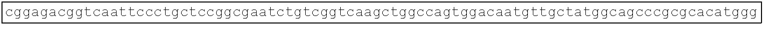

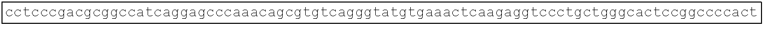

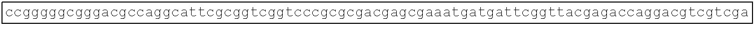

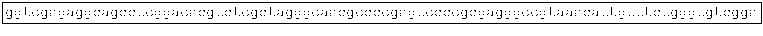

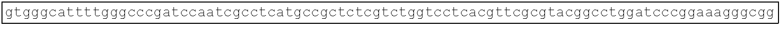

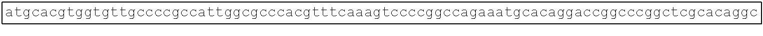

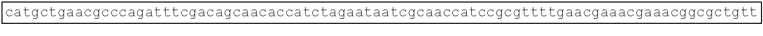

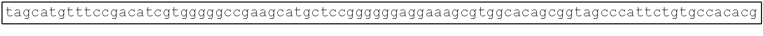

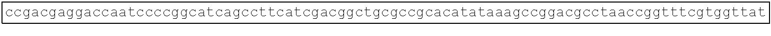

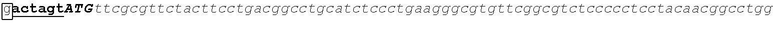

gcctgacgccccagatgggctgggacaactggaacacgttcgcctgcgacgtctccgagcagctgctgctggacacggccga
ccgcatctccgacctgggcctgaaggacatgggctacaagtacatcatcctggacgactgctggtcctccggccgcgactccga
cggcttcctggtcgccgacgagcagaagttccccaacggcatgggccacgtcgccgaccacctgcacaacaactccttcctgtt
cggcatgtactcctccgcgggcgagtacacgtgcgccggctaccccggctccctgggccgcgaggaggaggacgcccagttct
tcgcgaacaaccgcgtggactacctgaagtacgacaactgctacaacaagggccagttcggcacgcccgagatctcctacca
ccgctacaaggccatgtccgacgccctgaacaagacgggccgccccatcttctactccctgtgcaactggggccaggacctga
ccttctactggggctccggcatcgcgaactcctggcgcatgtccggcgacgtcacggcggagttcacgcgccccgactcccgct
gcccctgcgacggcgacgagtacgactgcaagtacgccggcttccactgctccatcatgaacatcctgaacaaggccgccccc
atgggccagaacgcgggcgtcggcggctggaacgacctggacaacctggaggtcggcgtcggcaacctgacggacgacga
ggagaaggcgcacttctccatgtgggccatggtgaagtcccccctgatcatcggcgcgaacgtgaacaacctgaaggcctcct
cctactccatctactcccaggcgtccgtcatcgccatcaaccaggactccaacggcatccccgccacgcgcgtctggcgctacta
cgtgtccgacacggacgagtacggccagggcgagatccagatgtggtccggccccctggacaacggcgaccaggtcgtggc
gctgctgaacggcggctccgtgtcccgccccatgaacacgaccctggaggagatcttcttcgactccaacctgggctccaagaa
gctgacctccacctgggacatctacgacctgtgggcgaaccgcgtcgacaactccacggcgtccgccatcctgggccgcaaca
agaccgccaccggcatcctgtacaacgccaccgagcagtcctacaaggacggcctgtccaagaacgacacccgcctgttcgg
ccagaagatcggctccctgtcccccaacgcgatcctgaacacgaccgtccccgcccacggcatcgcgttctaccgcctgcgcccc

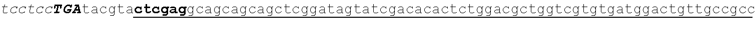

acacttgctgccttgacctgtgaatatccctgccgcttttatcaaacagcctcagtgtgtttgatcttgtgtgtacgcgcttttgcgagtt
gctagctgcttgtgctatttgcgaataccacccccagcatccccttccctcgtttcatatcgcttgcatcccaaccgcaacttatctacg
ctgtcctgctatccctcagcgctgctcctgctcctgctcactgcccctcgcacagccttggtttgggctccgcctgtattctcctggtact

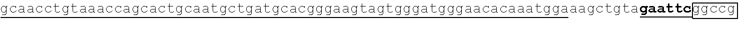

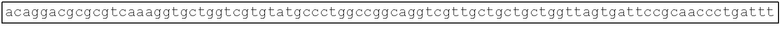

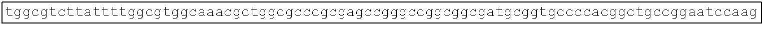

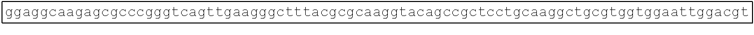

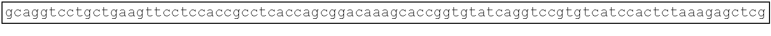

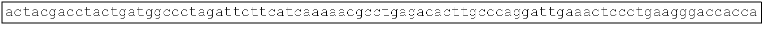

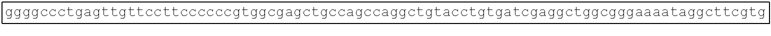

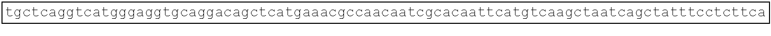

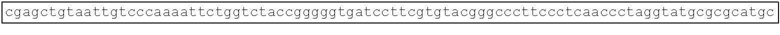

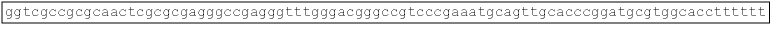

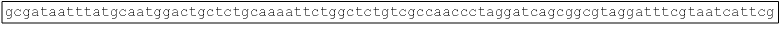

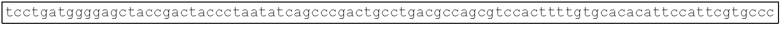

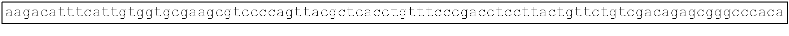

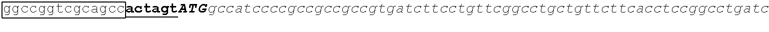

atcaacctgttccaggccctgtgcttcgtgctggtgtggcccctgtccaagaacgcctaccgccgcatcaaccgcgtgttcgccg
agctgctgctgtccgagctgctgtgcctgttcgactggtgggccggcgccaagctgaagctgttcaccgaccccgagaccttcc
gcctgatgggcaaggagcacgccctggtgatcatcaaccacatgaccgagctggactggatgctgggctgggtgatgggcca
gcacctgggctgcctgggctccatcctgtccgtggccaagaagtccaccaagttcctgcccgtgctgggctggtccatgtggttct
ccgagtacctgtacatcgagcgctcctgggccaaggaccgcaccaccctgaagtcccacatcgagcgcctgaccgactacccc
ctgcccttctggatggtgatcttcgtggagggcacccgcttcacccgcaccaagctgctggccgcccagcagtacgccgcctcct
ccggcctgcccgtgccccgcaacgtgctgatcccccgcaccaagggcttcgtgtcctgcgtgtcccacatgcgctccttcgtgccc
gccgtgtacgacgtgaccgtggccttccccaagacctcccccccccccaccctgctgaacctgttcgagggccagtccatcgtgc
tgcacgtgcacatcaagcgccacgccatgaaggacctgcccgagtccgacgacgccgtggcccagtggtgccgcgacaagtt
cgtggagaaggacgccctgctggacaagcacaacgccgaggacaccttctccggccaggaggtgcaccgcaccggctcccg
ccccatcaagtccctgctggtggtgatctcctgggtggtggtgatcaccttcggcgccctgaagttcctgcagtggtcctcctgga
agggcaaggccttctccgtgatcggcctgggcatcgtgaccctgctgatgcacatgctgatcctgtcctcccaggccgagcgctc

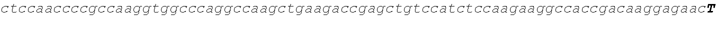

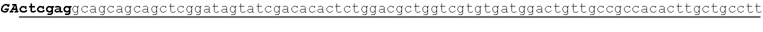

gacctgtgaatatccctgccgcttttatcaaacagcctcagtgtgtttgatcttgtgtgtacgcgcttttgcgagttgctagctgcttgtg
ctatttgcgaataccacccccagcatccccttccctcgtttcatatcgcttgcatcccaaccgcaacttatctacgctgtcctgctatcc
ctcagcgctgctcctgctcctgctcactgcccctcgcacagccttggtttgggctccgcctgtattctcctggtactgcaacctgtaaac

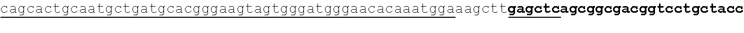

gtacgacgttgggcacgcccatgaaagtttgtataccgagcttgttgagcgaactgcaagcgcggctcaaggatacttgaactcct
ggattgatatcggtccaataatggatggaaaatccgaacctcgtgcaagaactgagcaaacctcgttacatggatgcacagtcgc
cagtccaatgaacattgaagtgagcgaactgttcgcttcggtggcagtactactcaaagaatgagctgctgttaaaaatgcactct
cgttctctcaagtgagtggcagatgagtgctcacgccttgcacttcgctgcccgtgtcatgccctgcgccccaaaatttgaaaaaag
ggatgagattattgggcaatggacgacgtcgtcgctccgggagtcaggaccggcggaaaataagaggcaacacactccgcttctt

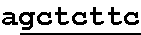

The sequence for all of the other LPAAT constructs are identical to that of pSZ3840 with the exception of the encoded LPAAT. The LPAAT sequence alone with flanking SpeI and XhoI restriction sites is provided for the remaining LPAAT constructs are shown below. The amino acid sequence of the LPAAT proteins is provided below.

pSZ3841/D2555 (CpaiLPAAT1)
SEQ ID NO: 20
actagt [symbol] gccatcccctccgccgccgtggtgttcctgttcggcctgc
tgttcttcacctccggcctgatcatcaacctgttccaggccttctgctt
cgtgctgatctcccccctgtccaagaacgcctaccgccgcatcaaccgc
gtgacgccgagctgctgcccctggagacctgtggctgttccactggtgc
gccggcgccaagctgaagctgttcaccgaccccgagaccttccgcctga
tgggcaaggagcacgccctggtgatcatcaaccacaagatcgagctgga
ctggatggtgggctgggtgctgggccagcacctgggctgcctgggctcc
atcctgtccgtggccaagaagtccaccaagttcctgcccgtgttcggct
ggtccctgtggttctccggctacctgttcctggagcgctcctgggccaa
ggacaagatcaccctgaagtcccacatcgagtccctgaaggactacccc
ctgcccttctggctgatcatcttcgtggagggcacccgcttcacccgca
ccaagctgctggccgcccagcagtacgccgcctcctccggcctgcccgt
gccccgcaacgtgctgatcccccacaccaagggcttcgtgtcctccgtg
tcccacatgcgctccttcgtgcccgccatctacgacgtgaccgtggcct
tccccaagacctcccccccccccaccatgctgaagctgacgagggccag
tccgtggagctgcacgtgcacatcaagcgccacgccatgaaggacctgc
ccgagtccgacgacgccgtggcccagtggtgccgcgacaagttcgtgga
gaaggacgccctgctggacaagcacaactccgaggacaccttctccggc
caggaggtgcaccacgtgggccgccccatcaaggccctgctggtggtga
tctcctgggtggtggtgatcatcttcggcgccctgaagttcctgctgtg
gtcctccctgctgtcctcctggaagggcaaggccactccgtgatcggcc
tgggcatcgtggccggcatcgtgaccctgctgatgcacatcctgatcct
gtcctcccaggccgagggctccaaccccgtgaaggccgcccccgccaag
ctgaagaccgagctgtcctcctccaagaaggtgaccaacaaggagaac [symbol] [symbol]
ctcgag
pSZ3842/D2556 (CigneaLPAAT1)
SEQ ID NO: 21
actagt [symbol] gccatcgccgccgccgccgtgatcttcctgttcggcctgc
tgttcttcgcctccggcatcatcatcaacctgttccaggccctgtgctt
cgtgctgatctggcccctgtccaagaacgtgtaccgccgcatcaaccgc
gtgacgccgagctgctgctgatggacctgctgtgcctgttccactggtg
ggccggcgccaagatcaagctgacaccgaccccgagaccttccgcctga
tgggcatggagcacgccctggtgatcatgaaccacaagaccgacctgga
ctggatggtgggctggatcctgggccagcacctgggctgcctgggctcc
atcctgtccatcgccaagaagtccaccaagttcatccccgtgctgggct
ggtccgtgtggactccgagtacctgttcctggagcgctcctgggccaag
gacaagtccaccctgaagtcccacatggagaagctgaaggactaccccc
tgcccttctggctggtgatcttcgtggagggcacccgcttcacccgcac
caagctgctggccgcccagcagtacgccgcctcctccggcctgcccgtg
ccccgcaacgtgctgatcccccacaccaagggcttcgtgtcctgcgtgt
ccaacatgcgctccacgtgcccgccgtgtacgacgtgaccgtggccttc
cccaagtcctcccccccccccaccatgctgaagctgttcgagggccagt
ccatcgtgctgcacgtgcacatcaagcgccacgccctgaaggacctgcc
cgagtccgacgacgccgtggcccagtggtgccgcgacaagttcgtggag
aaggacgccctgctggacaagcacaacgccgaggacaccttctccggcc
aggaggtgcaccacatcggccgccccatcaagtccctgctggtggtgat
cgcctgggtggtggtgatcatcttcggcgccctgaagttcctgcagtgg
tcctccctgctgtccacctggaagggcaaggccttctccgtgatcggcc
tgggcatcgccaccctgctgatgcacatgctgatcctgtcctcccaggc
cgagcgctccaaccccgccaaggtggccaag [symbol] ctcgag
pSZ3844/D2557 (ChookLPAAT1)
SEQ ID NO: 22
actagt [symbol] gccatcccctccgccgccgtggtgttcctgttcggcctgc
tgttcttcacctccggcctgatcatcaacctgttccaggccttctgctt
cgtgctgatctcccccctgtccaagaacgcctaccgccgcatcaaccgc
gtgacgccgagctgctgcccctggagacctgtggctgttccactggtgc
gccggcgccaagctgaagctgttcaccgaccccgagaccttccgcctga
tgggcaaggagcacgccctggtgatcatcaaccacaagatcgagctgga
ctggatggtgggctgggtgctgggccagcacctgggctgcctgggctcc
atcctgtccgtggccaagaagtccaccaagttcctgcccgtgttcggct
ggtccctgtggttctccgagtacctgttcctggagcgctcctgggccaa
ggacaagatcaccctgaagtcccacatcgagtccctgaaggactacccc
ctgcccttctggctgatcatcttcgtggagggcacccgcttcacccgca
ccaagctgctggccgcccagcagtacgccgcctcctccggcctgcccgt
gccccgcaacgtgctgatcccccacaccaagggcttcgtgtcctccgtg
tcccacatgcgctccttcgtgcccgccatctacgacgtgaccgtggcct
tccccaagacctcccccccccccaccatgctgaagctgacgagggccag
tccgtggagctgcacgtgcacatcaagcgccacgccatgaaggacctgc
ccgagtccgacgacgccgtggcccagtggtgccgcgacaagttcgtgga
gaaggacgccctgctggacaagcacaactccgaggacaccttctccggc
caggaggtgcaccacgtgggccgccccatcaaggccctgctggtggtga
tctcctgggtggtggtgatcatcttcggcgccctgaagttcctgctgtg
gtcctccctgctgtcctcctggaagggcaaggccactccgtgatcggcc
tgggcatcgtggccggcatcgtgaccctgctgatgcacatcctgatcct
gtcctcccaggccgagggctccaaccccgtgaaggccgcccccgccaag
ctgaagaccgagctgtcctcctccaagaaggtgaccaacaaggagaac [symbol] [symbol]
ctcgag

To determine the impact of the CpauLPAAT1, CigneaLPAAT1, ChookLPAAT1, and CpaiLPAAT1 genes on mid-chain fatty acid accumulation, the above constructs containing the codon optimized CpauLPAAT1, CigneaLPAAT1, ChookLPAAT1, and CpaiLPAAT1 genes were transformed into strain S6511. Primary transformants were clonally purified and grown under standard lipid production conditions at pH7.0 (all the strains require growth at pH 7.0 to allow for maximal expression of the LPAAT gene driven by the pH-regulated AMT3 promoter). The resulting profiles from a set of representative clones arising from these transformations are shown in Table 6.

TABLE 6
Transformants of pSZ3840 (CpauLPAAT1), pSZ3841 (CpaiLPAAT1),
pSZ3842 (CigneaLPAAT1), and pSZ3844 (ChookLPAAT1). The fatty acid profiles for
transgenic strains expressing LPAATs derived from C. paucipetala, C. painteri, C. ignea, and
C. hookeriana.

| | Sample ID | C8:0 | C10:0 | C12:0 | C14:0 | C16:0 | C18:0 | C18:1 | C18:2 | C18:3 a |
|---|---|---|---|---|---|---|---|---|---|---|
| Parent | S6511a | 14.4 | 27.7 | 0.6 | 1.3 | 8.8 | 1.6 | 38.2 | 5.4 | 0.4 |
| | S6511b | 14.5 | 27.7 | 0.6 | 1.3 | 8.6 | 1.6 | 38.4 | 5.3 | 0.4 |
| pSZ3840 CpauLPAAT1 | S6511; T792; D2554-20 | 16.6 | 29.9 | 0.7 | 1.3 | 8.0 | 1.0 | 35.2 | 5.2 | 0.5 |
| | S6511; T792; D2554-17 | 14.6 | 28.7 | 0.6 | 1.3 | 8.4 | 1.7 | 37.1 | 5.7 | 0.5 |
| | S6511; T792; D2554-41 | 15.2 | 28.5 | 0.7 | 1.3 | 8.3 | 1.4 | 37.5 | 5.2 | 0.4 |
| | S6511; T792; D2554-35 | 14.7 | 28.4 | 0.6 | 1.3 | 8.6 | 1.6 | 37.3 | 5.6 | 0.5 |
| | S6511; T792; D2554-27 | 15.2 | 27.6 | 0.7 | 1.3 | 9.5 | 1.5 | 37.1 | 5.1 | 0.4 |
| pSZ3841 CpaiLPAAT1 | S6511; T792; D2555-34 | 17.3 | 29.5 | 0.7 | 1.3 | 7.8 | 1.2 | 35.1 | 5.1 | 0.4 |
| | S6511; T792; D2555-43 | 17.5 | 29.1 | 0.7 | 1.3 | 8.0 | 0.9 | 35.4 | 5.0 | 0.5 |
| | S6511; T792; D2555-10 | 15.7 | 28.3 | 0.7 | 1.3 | 8.6 | 1.6 | 36.2 | 5.7 | 0.5 |
| | S6511; T792; D2555-22 | 16.0 | 27.9 | 0.7 | 1.3 | 8.4 | 0.9 | 37.8 | 5.0 | 0.4 |
| | S6511; T792; D2555-44 | 15.3 | 27.5 | 0.6 | 1.3 | 8.1 | 1.8 | 38.2 | 5.4 | 0.4 |
| pSZ3842 CigneaLPAAT1 | S6511; T792; D2556-38 | 16.2 | 29.2 | 0.7 | 1.3 | 8.1 | 1.3 | 36.1 | 5.2 | 0.5 |
| | S6511; T792; D2556-22 | 14.3 | 28.5 | 0.7 | 1.3 | 8.5 | 1.6 | 37.6 | 5.7 | 0.5 |
| | S6511; T792; D2556-44 | 13.6 | 28.4 | 0.7 | 1.4 | 9.0 | 1.5 | 36.3 | 6.7 | 0.7 |
| | S6511; T792; D2556-14 | 14.1 | 28.0 | 0.6 | 1.3 | 8.6 | 1.7 | 38.0 | 5.6 | 0.5 |
| | S6511; T792; D2556-36 | 14.3 | 28.0 | 0.6 | 1.3 | 8.6 | 1.7 | 37.9 | 5.7 | 0.5 |
| pSZ3844 ChookLPAAT1 | S6511; T792; D2557-47 | 15.8 | 29.3 | 0.7 | 1.3 | 8.2 | 1.2 | 36.5 | 5.0 | 0.5 |
| | S6511; T792; D2557-24 | 16.8 | 28.8 | 0.7 | 1.3 | 8.1 | 1.2 | 35.8 | 5.4 | 0.5 |
| | S6511; T792; D2557-30 | 15.2 | 28.3 | 0.7 | 1.3 | 8.5 | 1.6 | 36.8 | 5.7 | 0.5 |
| | S6511; T792; D2557-39 | 14.7 | 28.2 | 0.7 | 1.3 | 8.7 | 1.5 | 37.3 | 5.7 | 0.5 |
| | S6511; T792; D2557-26 | 15.3 | 27.7 | 0.7 | 1.4 | 8.7 | 0.9 | 37.7 | 5.4 | 0.5 |

The transformants in Table 6 display a marked increase in the production of C8:0 and C10:0 fatty acids upon expression of the heterologous LPAATs. To determine if expression of the heterologous LPAAT genes affected the regiospecificity of fatty acids at the sn-2 position, we analyzed TAGs from representative D2554 (CpauLPAAT1), D2555 (CpaiLPAAT1), D2556 (CigneaLPAAT1), and D2557 (ChookLPAAT1) strains utilizing the porcine pancreatic lipase method. Cells were grown under conditions to maximize midchain fatty acid levels and to generate sufficient biomass for TAG analysis. TAG and sn-2 profiles are shown in Table 7.

Table 7:

Inclusion of C8:0 and C10:0 fatty acids at the sn-2 position of TAGs. Selected transformants were subjected to porcine pancreatic lipase determination of fatty acid inclusion at the sn-2 position. The general fatty acid distribution in triacylglycerols (TAG) is shown to indicate fatty acid abundance for each transformant. In addition, the sn-2-specific distribution is shown. Numbers highlighted in bold and italic reflect significantly increased inclusion of the noted fatty acid compared to the parent S6511.

TABLE 7

| | | | | S6511; T792; | | S6511; T792; | | S6511; T792; | | S6511; T792; | |
|---|---|---|---|---|---|---|---|---|---|---|---|
| | | | | D2554-20 | | D2555-34 | | D2556-38 | | D2557-24 | |
| Strain: | | S6511 | | (CpauLPAAT1) | | (CpaiLPAAT1) | | (CigneaLPAAT1) | | (ChookLPAAT1) | |
| Analysis | | TAG | sn-2 | TAG | sn-2 | TAG | sn-2 | TAG | sn-2 | TAG | sn-2 |
| Fatty Acid | C8:0 | 14.4 | 8.5 | 16.6 | 12.8 | 17.3 | 22.3 | 16.2 | 10.0 | 16.8 | 29.1 |
| (area %) | C10:0 | 27.7 | 26.4 | 29.9 | 39.0 | 29.5 | 22.2 | 29.2 | 36.2 | 28.8 | 19.4 |
| | C12:0 | 0.6 | 0.4 | 0.7 | 0.3 | 0.7 | 0.4 | 0.7 | 0.4 | 0.7 | 0.3 |
| | C14:0 | 1.3 | 1.0 | 1.3 | 1.0 | 1.3 | 0.9 | 1.3 | 1.2 | 1.3 | 0.9 |
| | C16:0 | 8.8 | 0.9 | 8.0 | 1.1 | 7.8 | 1.1 | 8.1 | 1.2 | 8.1 | 0.9 |
| | C18:0 | 1.6 | 0.2 | 1.0 | 0.4 | 1.2 | 0.5 | 1.3 | 0.5 | 1.2 | 0.3 |
| | C18:1 | 38.2 | 52.5 | 35.2 | 37.8 | 35.1 | 43.6 | 36.1 | 42.2 | 35.8 | 40.7 |
| | C18:2 | 5.4 | 8.9 | 5.2 | 6.2 | 5.1 | 7.9 | 5.2 | 7.0 | 5.4 | 7.1 |
| | C18:3 α | 0.4 | 0.8 | 0.5 | 0.7 | 0.4 | 0.9 | 0.5 | 0.8 | 0.5 | 0.7 |
| | C8 + C10 | 42.2 | 34.9 | 46.4 | 51.8 | 46.8 | 44.5 | 45.5 | 46.1 | 45.6 | 48.5 |
| | sum | | | | | | | | | | |

As disclosed in Table 7, the CpauLPAAT1 and CigneaLPAAT1 genes show remarkable specificity towards C10:0 fatty acids. D2554-20 exhibits 39.0% of C10:0 in the sn-2 position versus just 26.4% in the S6511 base strain without the heterologous LPAAT, demonstrating a 1.5 fold increase in C10:0 inclusion at the sn-2 position. D2556-38 exhibits 36.2% of C10:0 in the sn-2 position versus 26.4% in the S6511 base strain, demonstrating a 1.4 fold increase in C10:0 inclusion at the sn-2 position. Although there is a small increase in C8:0 levels in the D2554-20 and D2555-34 strains, the vast majority of sn-2 targeting is C10:0-specific. Similarly, CpaiLPAAT1 and ChookLPAAT1 show remarkable specificity towards C8:0 fatty acids. D2555-34 exhibits 22.3% C8:0 in the sn-2 position versus just 8.5% in the S6511 base strain without the heterologous LPAAT, demonstrating a 2.6 fold increase in C8:0 inclusion at the sn-2 position. D2557-24 exhibits 29.1% C8:0 in the sn-2 position versus 8.5%, demonstrating a 3.4 fold increase in C8:0 inclusion at the sn-2 position. We teach that CpauLPAAT1 and CigneaLPAAT1 are C10:0-specific LPAATs and that CpaiLPAAT1 and ChookLPAAT1 are C8:0-specific LPAATs. Knutzon D S, Lardizabal K D, Nelsen J S, Bleibaum J L, Davies H M, Metz J G (1995) Cloning of a coconut endosperm cDNA encoding a 1-acyl-sn-glycerol-3-phosphate acyltransferase that accepts medium-chain-length substrates. Plant Physiol 109:999-1006

### Amino Acid Sequences for Novel LPAAT Genes

CpauLPAAT1
SEQ ID NO: 23
MAIPAAAVIFLFGLLFFTSGLIINLFQALCFVLVWPLSKNAYRRINRV
FAELLLSELLCLFDWWAGAKLKLFTDPETFRLMGKEHALVIINHMTEL
DWMLGWVMGQHLGCLGSILSVAKKSTKFLPVLGWSMWFSEYLYIERSW
AKDRTTLKSHIERLTDYPLPFWMVIFVEGTRFTRTKLLAAQQYAASSG
LPVPRNVLIPRTKGFVSCVSHMRSFVPAVYDVTVAFPKTSPPPTLLNL
FEGQSIVLHVHIKRHAMKDLPESDDAVAQWCRDKFVEKDALLDKHNAE
DTFSGQEVHRTGSRPIKSLLVVISWVVVITFGALKFLQWSSWKGKAFS
VIGLGIVTLLMHMLILSSQAERSSNPAKVAQAKLKTELSISKKATDKEN
CprocLPAAT1
SEQ ID NO: 24
MAIPAAAVIFLFGLIFFASGLIINLFQALCFVLIWPISKNAYRRINRVF
AELLLSELLCLFDWWAGAKLKLFTDPETFRLMGKEHALVIINHMTELDW
MVGWVMGQHFGCLGSILSVAKKSTKFLPVLGWSMWFTEYLYIERSWNKD
KSTLKSHIERLKDYPLPFWLVIFAEGTRFTQTKLLAAQQYAASSGLPVP
RNVLIPRTKGFVSCVSHMRSFVPAVYDLTVAFPKTSPPPTLLNLFEGQS
VVLHVHIKRHAMKDLPESDDEVAQWCRDKFVEKDALLDKHNAEDTFSGQ
ELQHTGRRPIKSLLVVISWVVVIAFGALKFLQWSSWKGKAFSVIGLGIV
TLLMHMLILSSQAERSKPAKVAQAKLKTELSISKTVTDKEN
CprocLPAAT1b
SEQ ID NO: 25
MAIPAAAVIFLFGLIFFASGLIINLFQALCFVLIWPISKNAYRRINRVF
AELLLSELLCLFDWWAGAKLKLFTDPETFRLMGKEHALVIINHMTELDW
MVGWVMGQHFGCLGSILSVAKKSTKFLPVLGWSMWFTEYLYIERSWNKD
KSTLKSHIERLKDYPLPFWLVIFAEGTRFTQTKLLAAQQYAASSGLPVP
RNVLIPRTKGFVSCVSHMRSFVPAVYDLTVAFPKTSPPPTLLNLFEGQS
VVLHVHIKRHAMKDLPESDDEVAQWCRDKFVEK
CprocLPAAT2a
SEQ ID NO: 26
IVNLVQAVCFVLVRPLSKNTYRRINRVVAELLWLELVWLIDWWAGVKIK
VFTDHETFHLMGKEHALVICNHKSDIDWLVGWVLAQRSGCLGSTLAVMK
KSSKFLPVIGWSMWFSEYLFLERNWAKDESTLKSGLNRLKDYPLPFWLA
LFVEGTRFTRAKLLAAQQYAASSGLPVPRNVLIPRTKGFVSSVSHMRSF
VPAIYDVTVAIPKTSPPPTLIRMFKGQSSVLHVHLKRHVMKDLPESDDA
VAQWCRDIFVEKDALLDKHNADDTFSGQELQDTGRPIKSLLVVISWAVL
EVFGAVKFLQWSSLLSSWKGLAFSGIGLGIITLLMHILILFSQSERSTP
AKVAPAKAKIEGESSKTEMEKEK
CprocLPAAT2b
SEQ ID NO: 27
IVNLVQAVCFVLVRPLSKNTYRRINRVVAELLWLELVWLIDWWAGVKIK
VFTDHETFHLMGKEHALVICNHKSDIDWLVGWVLAQRSGCLGSTLAVMK
KSSKFLPVIGWSMWFSEYLFLERNWAKDESTLKSGLNRLKDYPLPFWLA
LFVEGTRFTRAKLLAAQQYAASSGLPVPRNVLIPRTKGFVSSVSHMRSF
VPAIYDVTVAIPKTSPPPTLIRMFKGQSSVLHVHLKRHVMKDLPESDDA
VAQWCRDIFVEKDALLDKHNADDTFSGQELQDTGRPIKSLLV
CpaiLPAAT1
SEQ ID NO: 28
MAIPSAAVVFLFGLLFFTSGLIINLFQAFCFVLISPLSKNAYRRINRVF
AELLPLEFLWLFHWCAGAKLKLFTDPETFRLMGKEHALVIINHKIELDW
MVGWVLGQHLGCLGSILSVAKKSTKFLPVFGWSLWFSGYLFLERSWAKD
KITLKSHIESLKDYPLPFWLIIFVEGTRFTRTKLLAAQQYAASSGLPVP
RNVLIPHTKGFVSSVSHMRSFVPAIYDVTVAFPKTSPPPTMLKLFEGQS
VELHVHIKRHAMKDLPESDDAVAQWCRDKFVEKDALLDKHNSEDTFSGQ
EVHHVGRPIKALLVVISWVVVIIFGALKFLLWSSLLSSWKGKAFSVIGL
GIVAGIVTLLMHILILSSQAEGSNPVKAAPAKLKTELSSSKKVTNKEN
ChookLPAAT1
SEQ ID NO: 29
MAIPSAAVVFLFGLLFFTSGLIINLFQAFCFVLISPLSKNAYRRINRVF
AELLPLEFLWLFHWCAGAKLKLFTDPETFRLMGKEHALVIINHKIELDW
MVGWVLGQHLGCLGSILSVAKKSTKFLPVFGWSLWFSEYLFLERSWAKD
KITLKSHIESLKDYPLPFWLIIFVEGTRFTRTKLLAAQQYAASSGLPVP
RNVLIPHTKGFVSSVSHMRSFVPAIYDVTVAFPKTSPPPTMLKLFEGQS
VELHVHIKRHAMKDLPESDDAVAQWCRDKFVEKDALLDKHNSEDTFSGQ
EVHHVGRPIKALLVVISWVVVIIFGALKFLLWSSLLSSWKGKAFSVIGL
GIVAGIVTLLMHILILSSQAEGSNPVKAAPAKLKTELSSSKKVTNKEN
ChookLPAAT2a
SEQ ID NO: 30
LSLLFFVSGLIVNLVQAVCFVLIRPLSKNTYRRINRVVAELLWLELVWL
IDWWAGVKIKVFTDHETFNLMGKEHALVVCNHKSDIDWLVGWVLAQRSG
CLGSTLAVMKKSSKFLPVIGWSMWFSEYLFLERSWAKDESTLKSGLKRL
KDYPLPFWLALFVEGTRFTQAKLLAAQQYAASSGLPVPRNVLIPRTKGF
VSSVSHMRSFVPAIYDVTVAIPKTSVPPTMLRIFKGQSSVLHVHLKRHL
MKDLPESDDAVAQWCRDIFVEKDALLDKHNAEDTFSGQELQDIGRPIKS
LLVVISWAVLVIFGAVKFLQWSSLLSSWKGLAFSGIGLGIVTLLMHILI
LFSQSERSTPAKVAPAKPKNEGESSKTEMEKEH
ChookLPAAT2b
SEQ ID NO: 31
QIKVFTDHETFNLMGKEHALVVCNHKSDIDWLVGWVLAQWSGCLGSTLA
VMKKSSKFLPVIGWSMWFSEYLFLERSWAKDESTLKSGLKRLKDYPLPF
WLALFVEGTRFTQAKLLAAQQYAASSGLPVPRNVLIPRTKGFVSSVSHM
RSFVPAIYDVTVAIPKTSVPPTMLRIFKGQSSVLHVHLKRHLMKDLPES
DDAVAQWCRDIFVEKDALLDKHNAEDTFSGQELQDIGRPIKSLLVVISW
AVLVIFGAVKFLQWSSLLSSWKGLAFSGIGLGIVTLLMHILILFSQSER
STPAKVAPAKLKKEGESSKPETDKQN
ChookLPAAT3a
SEQ ID NO: 32
LSLLFFVSGLIVNLVQAVCFVLIRPLLKNTYRRINRVVAELLWLELVWL
IDWWAGIKIKVFTDHETFHLMGKEHALVICNHKSDIDWLVGWVLAQRSG
CLGSTLAVMKKSSKFLPVIGWSMWFSEYLFLERNWAKDESTLKSGLNRL
KDYPLPFWLALFVEGTRFTRAKLLAAQQYAASSGLPVPRNVLIPRTKGF
VSSVSQMRSFVPAIYDVTVAIPKTSPPPTLLRMFKGQSSVLHVHLKRHL
MNDLPESDDAVAQWCRDIFVEKDALLDKHNAEDTFSGQELQDTGRPIKS
LLVVISWATLVVFGAVKFLQWSSLLSSWKGLAFSGIGLGIITLLMHILI
LFSQSERSTPAKVAPAKPKNEGESSKTEMEKEH
ChookLPAAT3b
SEQ ID NO: 33
LSLLFFVSGLIVNLVQAVCFVLIRPLLKNTYRRINRVVAELLWLELVWL
IDWWAGIKIKVFTDHETFHLMGKEHALVICNHKSDIDWLVGWVLAQRSG
CLGSTLAVMKKSSKFLPVIGWSMWFSEYLFLERNWAKDESTLKSGLNRL
KDYPLPFWLALFVEGTRFTRAKLLAAQQYAASSGLPVPRNVLIPRTKGF
VSSVSQMRSFVPAIYDVTVAIPKTSPPPTLLRMFKGQSSVLHVHLKRHL
MNDLPESDDAVAQWCRDIFVEKDALLDKHNAEDTFSGQELQDIGRPIKS
LLVVISWAVLEIFGAVKFLQWSSLLSSWKGLAFSGIGLGIVTLLMHILI
LFSQSERSTPAKVAPAKPKKEGESSKPETDKEN
CigneaLPAAT1
SEQ ID NO: 34
MAIAAAAVIFLFGLLFFASGIIINLFQALCFVLIWPLSKNVYRRINRVF
AELLLMDLLCLFHWWAGAKIKLFTDPETFRLMGMEHALVIMNHKTDLDW
MVGWILGQHLGCLGSILSIAKKSTKFIPVLGWSVWFSEYLFLERSWAKD
KSTLKSHMEKLKDYPLPFWLVIFVEGTRFTRTKLLAAQQYAASSGLPVP
RNVLIPHTKGFVSCVSNMRSFVPAVYDVTVAFPKSSPPPTMLKLFEGQS
IVLHVHIKRHALKDLPESDDAVAQWCRDKFVEKDALLDKHNAEDTFSGQ
EVHHIGRPIKSLLVVIAWVVVIIFGALKFLQWSSLLSTWKGKAFSVIGL
GIATLLMHMLILSSQAERSNPAKVAK
CigneaLPAAT2
SEQ ID NO: 35
MAIAAAAVIFLFGLLFFASGIIINLFQALCFVLIWPLSKNVYRRINRVF
AELLLMDLLCLFHWWAGAKIKLFTDPETFRLMGMEHALVIMNHKTDLDW
MVGWILGQHLGCLGSILSIAKKSTKFIPVLGWSVWFSEYLFLERSWAKD
ESTLKSGLNRLKDYPLPFWLALFVEGTRFTRAKLLAAQQYAASSGLPVP
KNVLIPRTKGFVSSVSHMRSFVPAIYDVTVAIPKTSAPPTLLRMFKGQS
SVLHVHLKRHLMKDLPESDDAVAQWCRDIFVEKDALLDKHNAEDTFSGQ
ELHDIGRPVKSLLVVISWAMLVVFGAVKFLQWSSLLSSWKGLAFSGIGL
GIITLLMHILILFSQSERSTPAKVAPAKQKNNEGESSKTEMEKEH
DeLPAAT1
SEQ ID NO: 36
SGLVVNLIQAFFFVLVRPFSKNAYRKINRVVAELLWLELIWLIDWWAGV
KIQLYTDPETFKLMGKEHALVICNHKSDIDWLVGWILAQRSGCLGSALA
VMKKSSKFLPVIGWSMWFSEYLFLERSWAKDENTLKSGFQRLRDFPHAF
WLALFVEGTRFTQAKLLAAQEYASSMGLPAPRNVLIPRTKGFVTAVTHM
RPFVPAVYDVTLAIPKTSPPPTMLRLFKGQSSVVHIHLKRHLMSDLPKS
DDSVAQWCKDAFVVKDNLLDKHKENDSFGDGVLQDTGRPLNSLVVVISW
ACLLIFGALKFFQWSSILSSWKGLAFSAVGLGIVTVLMQILIQFSQSER
SNRPMPSKHAK
DeLPAAT2
SEQ ID NO: 37
MAIPTAAYVVPLGAIFFFSGLLVNLIQAFFFITVWPLSKKTYIRINKVI
VELLWLEFVWLADWWAGLKIEVYADAETFQLMGKEHALVICNHKSDIDW
LVGWILAQRAGCLGSSFAVTKKSARYLPVVGWSIWFSGAIFLERSWEKD
ENTLKAGFQRLREFPCAFWLGLFVEGTRFTQAKLLAAQEYASTMGLPFP
RNVLIPRTKGFIAAVNHMREFVPAIYDLTFAFPKDSPPPTMLRLLKGQP
SVVHVHIKRHLMKDLPEKNEAVAQWCKDVFLVKDKLLDKHKDDGSFGDG
ELHEIGRPLKSLVVVTTWACLLILGTLKFLLWSSLLSSWKGLIFSATGL
AVLTVLMQFLIQSTQSERSNPASLSK
CerLPAAT1a
SEQ ID NO: 38
LGLLFFISGLAVNLIQAVCFVFLRPLSKNTYRKINRVLAELLWLQLVWL
VDWWAGVKIKVFADRESFNLMGKEHALVICNHKSDIDWLVGWVLAQRSG
CLGSSLAVMKKSSKFLPVIGWSMWFSEYLFLERSWAKDESTLKEGLRRL
KDFPRPFWLALFVEGTRFTQAKLLAAQEYATSQGLPVPRNVLIPRTKVH
VHVKRHLMKELPETDEAVAQWCKDLFVEKDKLLDKHVAEDTFSDQPLQD
IGRPVKPLLVVSSWACLVAYGALKFLQWSSLLSSWKGIAVSAVALAIVT
ILMQIMILFSQSERSIPAKVA
CerLPAAT1b
SEQ ID NO: 39
LGLLFFISGLAVNLIQAVCFVFLRPLSKNTYRKINRVLAELLWLQLVWL
VDWWAGVKIKVFADRESFNLMGKEHALVICNHKSDIDWLVGWVLAQRSG
CLGSSLAVMKKSSKFLPVIGWSMWFSEYLFLERSWAKDESTLKEGLRRL
KDFPRPFWLALFVEGTRFTQAKLLAAQEYATSQGLPVPRNVLIPRTKGF
VSAVSHMRSFVPAVYDMTVAIPKSSPSPTMLRLFKGQSSVVHVHVKRHL
MKELPETDEAVAQWCKDLFVEKDKLLDKHVAEDTFSDQPLQDIGRPVKP
LLVVSSWACLVAYGALKFLQWSSLLSSWKGIAVSAVALAIVTILMQIMI
LFSQSERSIPTKVA
CerLPAAT2a
SEQ ID NO: 40
MAIAAAAVVFLFGLLFFTSGLIINLAQAVCFVLIWPLSKNAYRRINRVF
AELLLLELLWLFHWRAGAKLKLFADPETFRLFGKEHALVICNHRTDLDW
MVGWVLGQHFGCLGSILSVAKKSTKFLPVLGWSMWFSEYLFLERSWAKD
KSTLKSHTERLKDYPLPFWLGIFVEGTRFTRAKLLAAQQYAASSGLPVP
RNVLIPHTKLHVHIKRYAMKDLPESDDAVAQWCRDIYVEKDAFLDKHNA
EDTFSGQEVHHIGRPIKSLLVVISWVVVIIFGALKFLRWSSLLSSWKGK
AFSVIGLGIVTLLVNILILSSQAERSNPAKVAPAKLKTELSPSKKVTNK
EN
CerLPAAT2b
SEQ ID NO: 41
MAIAAAAVVFLFGLLFFTSGLIINLAQAVCFVLIWPLSKNAYRRINRVF
AELLLLELLWLFHWRAGAKLKLFADPETFRLFGKEHALVICNHRTDLDW
MVGWVLGQHFGCLGSILSVAKKSTKFLPVLGWSMWFSEYLFLERSWAKD
KSTLKSHTERLKDYPLPFWLGIFVEGTRFTRAKLLAAQQYAASSGLPVP
RNVLIPHTKGFVSSMSHMRSFVPAVYDLTVAFPKTSPPPTLLKLFEGQS
VVLHVHIKRYAMKDLPESDDAVAQWCRDIYVEKDAFLDKHNAEDTFSGQ
EVHHIGRPIKSLLVVISWVVVIIFGALKFLRWSSLLSSWKGKAFSVIGL
GIVTLLVNILILSSQAERSNPAKVAPAKLKTELSPSKKVTNKEN
BrLPAAT1a
SEQ ID NO: 42
AAAVIVPLGILFFISGLVVNLLQAICYVLIRPLSKNTYRKINRVVAETL
WLELVWIVDWWAGVKIQVFADNETFNRMGKEHALVVCNHRSDIDWLVGW
ILAQRSGCLGSALAVMKKSSKFLPVIGWSMWFSEYLFLERNWAKDESTL
KSGLQRLNDFPRPFWLALFVEGTRFTEAKLKAAQEYAASSELPVPRNVL
IPRTKGFVSAVSNMRSFVPAIYDMTVAIPKTSPPPTMLRLFKGQPSVVH
VHIKCHSMKDLPESDDAIAQWCRDQFVAKDALLDKHIAADTFPGQQEQN
IGRPIKSLAVVLSWSCLLILGAMKFLHWSNLFSSWKGIAFSALGLGIIT
LCMQILIRSSQSERSTPAKVVPAKPKDNHNDSGSSSQTE
BrLPAAT1b
SEQ ID NO: 43
AAAVIVPLGILFFISGLVVNLLQAVCYVLVRPMSKNTYRKINRVVAETL
WLELVWIVDWWAGVKIQVFADDETFNRMGKEHALVVCNHRSDIDWLVGW
ILAQRSGCLGSALAVMKKSSKFLPVIGWSMWFSEYLFLERNWAKDESTL
KSGLQRLNDFPRPFWLALFVEGTRFTEAKLKAAQEYAASSELPVPRNVL
IPRTKGFVSAVSNMRSFVPAIYDMTVAIPKTSPPPTMLRLFKGQPSVVH
VHIKCHSMKDLPESDDAIAQWCRDQFVAKDALLDKHIAADTFPGQQEQN
IGRPIKSLAVVLSWSCLLILGAMKFLHWSNLFSSWKGIAFSALGLGIIT
LCMQILIRSSQSERSTPAKVVPAKPKDNHNDSGSSSQTE
BrLPAAT1e
SEQ ID NO: 44
MAIAAAVIVPLGLLFFISGLLMNLLQAICYVLVRPLSKNTYRKINRVVA
ETLWLELVWIVDWWAGVKIKVFADNETFSRMGKEHALVVCNHRSDIDWL
VGWILAQRSGCLGSALAVMKKSSKFLPVIGWSMWFSEYLFLERNWAKDE
STLKSGLQRLNDFPRPFWLALFVEGTRFTEAKLKAAQEYAASSELPVPR
NVLIPRTKGFVSAVSNMRSFVPAIYDMTVAIPKTSPPPTMLRLFKGQPS
VVHVHIKCHSMKDLPESDDAIAQWCRDQFVAKDALLDKHIAADTFPGQQ
EQNIGRPIKSLAVVLSWSCLLILGAMKFLHWSNLFSSWKGIAFSALGLG
IITLCMQILIRSSQSERSTPAKVVPAKPKDNHNDSGSSSQTE
BjLPAAT1a
SEQ ID NO: 45
INLVVAETLWLELVWIVDWWAGVKIQVFADDETFNRMGKEHALVVCNHR
SDIDWLVGWILAQRSGCLGSALAVMKKSSKFLPVIGWSMWFSEYLFLER
NWAKDESTLKSGLQRLNDFPRPFWLALFVEGTRFTEAKLKAAQEYAASS
ELPVPRNVLIPRTKGFVSAVSNMRSFVPAIYDMTVAIPKTSPPPTMLRL
FKGQPSVVHVHIKCHSMKDLPESDDAIAQWCRDQFVAKDALLDKHIAAD
TFPGQKEQNIGRPIKSLAVSLIKTFPWLHPHQLTNIFVLFQVVVSWACL
LTLGAMKFLHWSNLFSSWKGIALSAFGLGIITLCMQILIRSSQSERSTP
AKVAPAKPK
BjLPAAT1b
SEQ ID NO: 46
INLVVAETLWLELVWIVDWWAGVKIQVFADDETFNRMGKEHALVVCNHR
SDIDWLVGWILAQRSGCLGSALAVMKKSSKFLPVIGWSMWFSEYLFLER
NWAKDESTLKSGLQRLNDFPRPFWLALFVEGTRFTEAKLKAAQEYAASS
ELPVPRNVLIPRTKGFVSAVSNMRSFVPAIYDMTVAIPKTSPPPTMLRL
FKGQPSVVHVHIKCHSMKDLPEPEDEIAQWCRDQFVAKDALLDKHIAAD
TFPGQKEQNIGRPIKSLAVVVSWACLLTLGAMKFLHWSNLFSSWKGIAL
SAFGLGIITLCMQILIRSSQSERSTPAKVAPAKPK
BjLPAAT1e
SEQ ID NO: 47
INLVVAETLWLELVWIVDWWAGVKIQVFADDETFNRMGKEHALVVCNHR
SDIDWLVGWILAQRSGCLGSALAVMKKSSKFLPVIGWSMWFSEYLFLER
NWAKDESTLKSGLQRLNDFPRPFWLALFVEGTRFTEAKLKAAQEYAASS
ELPVPRNVLIPRTKGFVSAVSNMRSFVPAIYDMTVAIPKTSPPPTMLRL
FKGQPSVVHVHIKCHSMKDLPESDDAIAQWCRDQFVAKDALLDKHIAAD
TFPGQQEQNIGRPIKSLAVVLSWSCLLILGAMKFLHWSNLFSSWKGIAF
SALGLGIITLCMQILIRSSQSERSTPAKVVPAKPKDNHNDSGSSSQTE
BjLPAAT1d
SEQ ID NO: 48
INLVVAETLWLELVWIVDWWAGVKIQVFADDETFNRMGKEHALVVCNHR
SDIDWLVGWILAQRSGCLGSALAVMKKSSKFLPVIGWSMWFSEYLFLER
NWAKDESTLKSGLQRLNDFPRPFWLALFVEGTRFTEAKLKAAQEYAASS
ELPVPRNVLIPRTKGFVSAVSNMRSFVPAIYDMTVAIPKTSPPPTMLRL
FKGQPSVVHVHIKCHSMKDLPESDDAIAQWCRDQFVAKDALLDKHIAAD
TFPGQQEQNIGRPIKSLAVSLS
CeLPAAT1a
SEQ ID NO: 49
MAIGVAAIVVPLGLLFILSGLMVNLIQAICFILVRPLSKNMYRRVNRVV
VELLWLELIWLIDWWGGVKVDVYADSETFQSLGKEHALVVSNHRSDIDW
LVGWVLAQRSGCLGSTLAVMKKSSKFLPVIGWSMWFSEYVFLERSWAKD
ESTLKSGLRRLKDFPRPFWLALFVEGTRFTQAKLLAAREYAASTGLPIP
RNVLIPRTKGFVSAVSNMRSFVPAIYDVTVAIPKTQPSPTMLRIFNRQP
SVVHVHIKRHSMNQLPQTDEGVGQWCKDIFVAKDALLDRHLAE
CcLPAAT1b
SEQ ID NO: 50
MAIGVAAIVVPLGLLFILSGLMVNLIQAICFILVRPLSKNMYRRVNRVV
VELLWLELIWLIDWWGGVKVDVYADSETFQSLGKEHALVVSNHRSDIDW
LVGWVLAQRSGCLGSTLAVMKKSSKFLPVIGWSMWFSEYVFLERSWAKD
ESTLKSGLRRLKDFPRPFWLALFVEGTRFTQAKLLAAREYAASTGLPIP
RNVLIPRTKGFVSAVSNMRSFVPAIYDVTVAIPKTQPSPTMLRIFNRQP
SVVHVHIKRHSMNQLPQTDEGVAQWCKDIFVAKDALLDRHLAEGKFDEK
EFKRIRRPIKSLLVISSWSFLLMFGVFKFLKWSALLSTWKGVAVSTTVL
LLVTVVMYMFILFSQSERSSPRKVAPSGPENG
UcLPAAT1a
SEQ ID NO: 51
MAIGVAAIVVPLGLLFILSGLIINLIQAICFILVRPLSKNMYRKVNRVV
VELLWLELIWLIDWWGGVKVDVYADSETFQSLGKEHALVVSNHRSDIDW
LVGWVLAQRSGCLGSTLAVMKKSSKFLPVIGWSMWFSEYVFLERSWAKD
ESTLKSGLQRLKDFPRPFWLALFVEGTRFTQAKLLAAQEYAASTGLPIP
RNVLIPRTKGFVSAVSNMRSFVPAIYDVTVAIPKTQPSPTMLRIFNRQP
SVVHVHIKRHSMNQLPQTDEGVAQWCKDIFVAKDALLDRHLAEGKFDEK
EFKLIRRPIKSLLVISSWSFLLMFGVFKFLKWSALLSTWKGVAVSTAVL
LLVTVVMYMFILFSQSERSSPRKVAPIGPENG
UcLPAAT1b
SEQ ID NO: 52
MAIGVAAIVVPLGLLFILSGLIINLIQAICFILVRPLSKNMYRKVNRVV
VELLWLELIWLIDWWGGVKVDVYADSETFQSLGKEHALVVSNHRSDIDW
LVGWVLAQRSGCLGSTLAVMKKSSKFLPVIGWSMWFSEYVFLERSWAKD
ESTLKSGLQRLKDFPRPFWLALFVEGTRFTQAKLLAAQEYAASTGLPIP
RNVLIPRTKGFVSAVSNMRSFVPAIYDVTVAIPKTQPSPTMLRIFNRQP
SVVHVHIKRHSMNQLPQTDEGVAQWCKDIFVAKDALLDRHLAE
LdLPAAT1
SEQ ID NO: 53
SLLFFMSGLVVNFIQAVFYVLVRPISKNTYRRINTLVAELLWLELVWVI
DWWAGVKVQLYTDTESFRLMGKEHALLICNHRSDIDWLIGWVLAQRCGC
LSSSIAVMKKSSKFLPVIGWSMWFSEYLFLERNWAKDENTLKSGLQRLN
DFPKPFWLALFVEGTRFTKAKLLAAQEYAASAGLPVPRNVLIPRTKGFV
SAVSNMRSFVPAIYDLTVAIPKTTEQPTMLRLFRGKSSVVHVHLKRHLM
KDLPKTDDGVAQWCKDQFISKDALLDKHVAEDTFSGLEVQDIGRPMKSL
VVVVSWMCLLCLGLVKFLQWSALLSSWKGMMITTFVLGIVTVLMHILIR
SSQSEHSTPAK
CaequLPAAT1a
SEQ ID NO: 54
QRSGCLGSTLAVMKKSSKFLPVIGWSMWFSEYLFLERSWAKDESTLKSG
LKRLKDYPLPFWLALFVEGTRFTQAKLLAAQQYAASSGLPVPRNVLIPR
PTKGFVSSVSHMRSFVAIYDVTVAIPKMSTPPTMLRIFKGQSSVLHVHL
KRHLMKDLPESDDAVAQWCRDIFVEKDALLDKHNAEDTFSGQELQDIGR
PVKSLLVVISWAVLVIFGAVKFLQWSSLLSSWKGLAFSGIGLGIVTLLM
HILILFSQSERSTPAKVAPAKPKKEGESSKTETEKEN
CaequLPAAT1b
SEQ ID NO: 55
DWWAGVKIKVFTDHETLSLMGKEHALVISNHKSDIDWLVGWVLAQRSGC
LGSTLAVMKKSSKFLPVIGWSMWFSEYLFLERSWAKDESTLKSGLKRLK
DYPLPFWLALFVEGTRFTQAKLLAAQQYAASSGLPVPRNVLIPRTKGFV
SSVSHMRSFVPAIYDVTVAIPKMSTPPTMLRIFKGQSSVLHVHLKRHLM
KDLPESDDAVAQWCRDIFVEKDALLDKHN
AEDTFSGQELQDIGRPVKSLLV
CaequLPAAT1e
SEQ ID NO: 56
DWWAGVKIKVFTDHETLSLMGKEHALVISNHKSDIDWLVGWVLAQRSGC
LGSTLAVMKKSSKFLPVIGWSMWFSEYLFLERSWAKDESTLKSGLKRLK
DYPLPFWLALFVEGTRFTQAKLLAAQQYAASSGLPVPRNVLIPRTKGFV
SSVSHMRSFVPAIYDVTVAIPKMSTPPTMLRIFKGQSSVLHVHLKRHLM
KDLPESDDAVAQWCRDIFVEKDALLDKHNAEDTFSGQELQDIGRPVKSL
LVVISWAVLVIFGAVKFLQWSSLLSSWKGLAFSGIGLGIVTLLMHILIL
FSQSERSTPAKVAPAKPKKEGESSKTETEKEN
CaequLPAAT1d
SEQ ID NO: 57
QRSGCLGSTLAVMKKSSKFLPVIGWSMWFSEYLFLERSWAKDESTLKSG
LKRLKDYPLPFWLALFVEGTRFTQAKLLAAQQYAASSGLPVPRNVLIPR
TKGFVSSVSHMRSFVPAIYDVTVAIPKMSTPPTMLRIFKGQSSVLHVHL
KRHLMKDLPESDDAVAQWCRDIFVEKDALLDKHNAEDTFSGQELQDIGR
PVKSLLV
CglutLPAAT1a
SEQ ID NO: 58
LSLLFFVSGLFVNLVQAVCFVLIRPFSKNTYRRINRVVAELLWLELVWL
IDWWAGVKIKVFTDHETLSLMGKEHALVISNHKSDIDWLVGWVLAQRSG
CLGSTLAVMKKSSKFLPVIGWSMWFSEYLFLERSWAKDESTLKSGLKRL
KDYPLPFWLALFVEGTRFTQAKLLAAQQYAASSGLPVPRNVLIPRTKGF
VSSVSHMRSFVPAIYDVTVAIPKMSTPPTMLRIFKGQSSVLHVHLKRHL
MKDLPESDDAVAQWCRDIFVEKDALLDKHNAEDTFSGQELQDIGRPVKS
LLVVISWAVLVIFGAVKFLQWSSLLSSWKGLAFSGIGLGIVTLLMHILI
LFSQSERSTPAKVAPAKPKKEGESSKTETEKEN
CglutLPAAT1b
SEQ ID NO: 59
QAVCFVLIRPFSKNTYRRINRVVAELLWLELVWLIDWWAGVKIKVFTDH
ETLSLMGKEHALVISNHKSDIDWLVGWVLAQRSGCLGSTLAVMKKSSKF
LPVIGWSMWFSEYLFLERSWAKDESTLKSGLKRLKDYPLPFWLALFVEG
TRFTQAKLLAAQQYAASSGLPVPRNVLIPRTKGFVSSVSHMRSFVPAIY
DVTVAIPKMSTPPTMLRIFKGQSSVLHVHLKRHLMKDLPESDDAVAQWC
RDIFVEKDALLDKHNAEDTFSGQELQDIGRPVKSLLVVISWAVLVIFGA
VKFLQWSSLLSSWKGLAFSGIGLGIVTLLMHILILFSQSERSTPAKVAP
AKPKKEGESSKTETEKEN
CprLPAAT1
SEQ ID NO: 60
MAIAAAAVVFLFGLLFFTSGLIINLAQAVCFVLIWPLSKNAYRRINRVF
AELLLLELLWLFHWRAGAKLKLFADPETFRLFGKEHALVICNHRTDLDW
MVGWVLGQHFGCLGSILSVAKKSTKFLPVLGWSMWFSEYLFLERSWAKD
KSTLKSHTERLKDYPLPFWLGIFVEGTRFTRAKLLAAQQYAASSGLPVP
RNVLIPHTKGFVSSMSHMRSFVPAVYDLTVAFPKTSPPPTLLKLFEGQS
VVLHVHIKRYAMKDLPESDDAVAQWCRDIYVEKDAFLDKHNAEDTFSGQ
EVHHIGRPIKSLLVVISWVVVIIFGALKFLRWSSLLSSWKGKAFSVIGL
GIVTLLVNILILSSQAERSNPAKVVPAKLKTELSPSKKVTNKEN
ChsLPAAT1
SEQ ID NO: 61
MAIPSAAVVFLFGLLFFASGLIINLVQAVCFVLIWPLSKNTCRRINIVF
QDMLLSELLWLFHWRAGAKLKFFTDPETYRHMGKEHALVITNHRTDLDW
MIGWVLGEHLGCLGSILSVVKKSTKFLPVLGWSMWFSEYLFLERNWAKD
KSTFKSHIERLEDFPQPFWFGIFVEGTRFTRAKLLAAQQYAASSGLPVP
RNVLIPHTKGFVSSVSHMRSFVPAVYETTMTFPKTSPPPTLLKLFEGQP
LVLHIHMKRHAMKDIPESDDAVAQWCRDKFVEKDALLDKHNAEDTFGGL
EVHIGRSIKSLMVVICWVVVIIFGALKFLQWSSLLSSWKGIAFIGIGLG
IVNLLVHVLILSSQAERSAPTKVAPAKLKTKLLSSKKITNKEN
ChsLPAAT2
SEQ ID NO: 62
MAIPSAAVVFLFGLLFFASGLIINLVQAVCFVLIWPLSKNTCRRINIVF
QDMLLSELLWLFHWRAGAKLKFFTDPETYRHMGKEHALVITNHRTDLDW
MIGWVLGEHLGCLGSILSVVKKSTKFLPVLGWSMWFSEYLFLERNWAKD
KSTFKSHIERLEDFPQPFWFGIFVEGTRFTRAKLLAAQQYAASSGLPVP
RNVLIPRTKGFVSSVSHMRSFVPAIYDVTVAIPKTSPPPTMLRMFKGQS
SVLHVHLKRHLMKDLPESDDAVAQWCRDIFVEKDALLDKHNAEDTFSGQ
ELQDIGRPIKSLVVVISWAALVVFGAVKFLQWSSLLSSWKGLAFSGIGL
GIITLLMHILILFSQSERSTPAKVAPAKPKREGESSKTEMDKEN
CcaleLPAAT1a
SEQ ID NO: 63
MAIPAAAVVFLFGLLFFPSGLIINLFQAVCFVLIWPFSRNTCRRINIVF
QEMLLSELLWLFHWRAGAKLKLFADPETYRHMGKEHALLITNHRTDLDW
MIGWALGQHLGCLGSILSVVKKSTKFLPSHIERLEDFPQPFWMAIFVEG
TRFTRAKLLAAQQYAASSGLPVPRNVLIPRTKGFVSCVSHMRSFVPAVY
ETTMTFPKTSPPPTLLKLFEGQPIVLHVHMKRHAMKDIPESDEAVAQWC
RDKFVEKDSLLDKHNAGDTFSCQEIHIGRPIKSLMVVISWVVVIIFGAL
KFLQWSSLLSSWKGIAFSGIGLGIVTLLVHILILSSQAERSTPAKVAPA
KLKTELSSSTKVTNKEN
CcaleLPAAT1b
SEQ ID NO: 64
MAIPAAAVVFLFGLLFFPSGLIINLFQAVCFVLIWPFSRNTCRRINIVF
QEMLLSELLWLFHWRAGAKLKLFADPETYRHMGKEHALLITNHRTDLDW
MIGWALGQHLGCLGSILSVVKKSTKFLPVLGWSMWFSEYLFLERNWAKD
KSTFKSHIERLEDFPQPFWMAIFVEGTRFTRAKLLAAQQYAASSGLPVP
RNVLIPRTKGFVSCVSHMRSFVPAVYETTMTFPKTSPPPTLLKLFEGQP
IVLHVHMKRHAMKDIPESDEAVAQWCRDKFVEKDSLLDKHNAGDTFSCQ
EIHIGRPIKSLMVVISWVVVIIFGALKFLQWSSLLSSWKGIAFSGIGLG
IVTLLVHILILSSQAERSTPAKVAPAKLKTELSSSTKVTNKEN
CcaleLPAAT2
SEQ ID NO: 65
LSLLFFVSGLIVNLVQAVCFVLIRPLSKNTYRRINRVVAELLWLELVWL
IDWWAGVKIKVFTDHETFRLMGTEHALVISNHKSDIDWLVGWVLAQRSG
CLGSTLAVMKKSSKFLPVIGWSMWFSEYLFLERSWAKDESTLKSGLNRL
KDYPLPFWLALFVEGTRFTRAKLLAAQQYAASSGLPVPRNVLIPRTKGF
VSSVSHMRSFVPAIYDVTVAIPKTSPPPTMLRMFKGQSSVLHVHLKRHL
MKDLPESDDAVAQWCRDIFVEKDALLDKHNAEDTFSGQELQDIGRPIKS
LVVVISWAALVVFGAVKFLQWSSLLSSWKGLAFSGIALGIITLLMHILI
LFSQSERSTPAKVAPAKPKKEGESSKTETDKEN
ChtLPAAT1a
SEQ ID NO: 66
MAIPAAAVIFLFSILFFASGLIINLVQAVCFVLIWPLSKNTCRRINLVF
QEMLLSELLGLFHWRAGAKLKLYTDPETYPLLGKEHALLMINHRTDLDW
MIGWVLGQHLGCLGSILSVVKKSTKFLPVLGWSMWFSEYLFLERNWAKD
KSTFKSHIERLEDFPQPFWMAIFVEGTRFTRAKLLAAQQYAASSGLPVP
RNVLIPHTKGFVSTVSHMRSFVPAVYDTTLTFPKTSPPPTLLNLFAGQP
IVLHIHIKRHAMKDIPESDDAVAQWCRDKFVEKDALLDKHNAEDAFSDQ
EFPISRSIKSLMVVISWVMVIIFGALKFLQWSSLLSSWKGKAFSVIAVG
IVTLLMHMSILSSQAERSNPAKVALPKLKTELPSSKKVLNKEN
ChtLPAAT1b
SEQ ID NO: 67
MAIPAAAVIFLFSILFFASGLIINLVQAVCFVLIWPLSKNTCRRINLVF
QEMLLSELLGLFHWRAGAKLKLYTDPETYPLLGKEHALLMINHRTDLDW
MIGWVLGQHLGCLGSILSVVKKSTKFLPVLGWSMWFSEYLFLERNWAKD
KSTFKSHIERLEDFPQPFWMAIFVEGTRFTRAKLLAAQQYAASSGLPVP
RNVLIPHTKGFVSTVSHMRSFVPAVYDTTLTFPKTSPPPTLLNLFAGQP
IVLHIHIKRHAMKDIPESDDAVAQWCRDKFVEKDALLDKHNAEDAFSDQ
EFPISRSIKSLMVVISWVMVIIFGALKFLQWSSLLSSWKGIAFSGIGLG
IVTLLMHILILSSQAERSTPAKVAQAKVKTELPSSTKVTNKGN
CwLPAAT1
SEQ ID NO: 68
MAIPAAAVIFLFGILFFASGLIINLVQAVCFVLIWPLSKNTCRRINLVF
QEMLLSELLWLFHWRAGAELKLFTDPETYRLLGKEHALVMTNHRTDLDW
MIGWVTGQHLGCLGSILSIAKKSTKFLPVLGWSMWFSEYLFLERNWAKD
KSTFKSHIERLEDFPQPFWMAIFVEGTRFTRAKLLAAQQYAASSGLPVP
RNVLIPHTKGFVSSVCHMRSFVPAVYDTTLTFPKNSPPPTLLNLFAGQP
IVLHIHIKRHAMKDMPKSDDAVAQWCRDKFVKKDALLDKHNTEDTFSDQ
EFPIGRPIKSLMVVISWVVVIIFGTLKFLQWSSLLSSWKGIAFSGIGLG
IVTLLVHILILSSQAERSTPPKVAPAKLKTELSSTTKVINKGN
CwLPAAT2b
SEQ ID NO: 69
LGLLFFVSGLIVNLVQAVCFVLIRPLSKNTYRRLNRVVAELLWLELVWL
IDWWAGVKIKVFTDHETFHLMGKEHALVICNHKSDIDWLVGWVLAQRSG
CLGSTLAVMKKSSKFLPVIGWSMWFSEYLFLERSWAKDESTLKSGLNRL
KDYPLPFWLALFVEGTRFTRAKLLAAQQYAASSGLPVPRNVLIPRTKGF
RVSSVSHMRSFVPAIYDVTVAIPKTSPPPTMLMFKGQSSVDALLDKHNA
DDTFSGQELHDIGRPIKSLLVVISWAVLVVFGAVKFLQWSSLLSSWKGI
AFSGIGLGIVTLLVHILILSSQAERSTSAKVAQAKVKTELSSSKKVKNK
GN
CwLPAAT2a
SEQ ID NO: 70
LGLLFFVSGLIVNLVQAVCFVLIRPLSKNTYRRLNRVVAELLWLELVWL
IDWWAGVKIKVFTDHETFHLMGKEHALVICNHKSDIDWLVGWVLAQRSG
CLGSTLAVMKKSSKFLPVIGWSMWFSEYLFLERSWAKDESTLKSGLNRL
KDYPLPFWLALFVEGTRFTRAKLLAAQQYAASSGLPVPRNVLIPRTKGF
VSSVSHMRSFVPAIYDVTVAIPKTSPPPTMLRMFKGQSSVLHVHLKRHL
MKDLPESDDAVAQWCRDIFVEKDVLLDKHNAEDTFSGQELQDIGRPVKS
LLVVISWTLLVIFGAVKFLQWSSLLSSWKGLAFSGIGLGIVTLLMHILI
LFSQSERSTPAKVAPAKPKKEGESSKMETDKEN
CgLPAAT1a
SEQ ID NO: 71
LAGWMGSSSGCLGSTLAVMKKSSKFLPVIGWSMWFSEYLFLERSWAKDE
STLKSGLNRLKDYPLPFWLALFVEGTRFTRAKLLAAQQYAASLGLPVPR
NVLIPRTKGFVSSVSHMRSFVPAIYDVTVAIPKTSPPPTMIRMFKGQSS
VLHVHLKRHVMKDLPESDDAVAQWCRDIFVEKDALLDKHNAEDTFSGQE
LQDTGRPIKSLLVVISWAVLEVFGAVKFLQWSSLLSSWKGLAFSGIGLG
IITLLMHILILFSQSERSTPAKVAPAKPKNEGESSKAEMEKEK
CgLPAAT1b
SEQ ID NO: 72
LAGWMGSSSGCLGSTLAVMKKSSKFLPVIGWSMWFSEYLFLERSWAKDE
STLKSGLNRLKDYPLPFWLALFVEGTRFTRAKLLAAQQYAASLGLPVPR
NVLIPRTKGFVSSVSHMRSFVPAIYDVTVAIPKTSPPPTMIRMFKGQSS
VLHVHLKRHVMKDLPESDDAVAQWCRDIFVEKDALLDKHNAEDTFSGQE
LQDTGRPIKSLLVRCFLVLSLIYLNGIMLKLRGPCLQVVISWAVLEVFG
AVKFLQWSSLLSSWKGLAFSGIGLGIITLLMHILILFSQSERSTPAKVA
PAKPKNEGESSKAEMEKEK
CgLPAAT1c
SEQ ID NO: 73
LAGWMGSSSGCLGSTLAVMKKSSKFLPVIGWSMWFSEYLFLERSWAKDE
STLKSGLNRLKDYPLPFWLALFVEGTRFTRAKLLAAQQYAASLGLPVPR
NVLIPRTKGFVSSVSHMRSFVPAIYDVTVAIPKTSPPPTMIRMFKGQSS
VLHVHLKRHVMKDLPESDDAVAQWCRDIFVEKDALLDKHNAEDTFSGQE
LQDTGRPIKSLLVVTSWAVLVISGAVKFLQWSSLLSSWKGLAFSGIGLG
IVTLLMHILILFSQSERSTPAKVAPAKPKKEGESSKTEKDKEN
CpalLPAAT1
SEQ ID NO: 74
LGLLFFVSGLIVNLVQAVCFVLIRPLSKNTYRRINRVVAELLWLELVWL
IDWWAGVKIKVFTDHETLSLMGKEHALVICNHKSDIDWLVGWVLAQRSG
CLGSTLAVMKKSSKFLPVIGWSMWFSEYLFLERSWAKDENTLKSGLNRL
KDYPLPFWLALFVEGTRFTRAKLLAAQQYATSSGLPVPRNVLIPRTKGF
VSSVSHMRSFVPAIYDVTVAIPKTSPPPTMLRMFKGQSSVLHVHLKRHL
MKDLPESDDAVAQWCRDIFVEKDALLDKHNAEDTFSGQELQDTGRPIKS
LLVVISWAVLVIFGAVKFLQWSSLLSSWKGLAFSGVGLGIITLLMHILI
LFSQSERSTPAKVAPAKPKKDGESSKTEIEKEN
CaLPAAT1
SEQ ID NO: 75
MAIAAAAVIVPVSLLFFVSGLIVNLVQAVCFVLIRPLFKNTYRRINRVV
AELLWLELVWLIDWWAGVKIKVFTDHETFHLMGKEHALVICNHKSDIDW
LVGWVLAQRSGCLGSTLAVMKKSSKFLPVIGWSMWFSEYLFLERNWAKD
ESTLKSGLNRLKDYPLPFWLALFVEGTRFTRAKLLAAQQYAASSGLPVP
RNVLIPRTKGFVSSVSHMRSFVPAIYDVTVAIPKTSPPPTLLRMFKGQS
SVLHVHLKRHQMNDLPESDDAVAQWCRDIFVEKDALLDKHNAEDTFSGQ
ELQDTGRPIKSLLIVISWAVLVVFGAVKFLQWSSLLSSWKGLAFSGIGL
GVITLLMHILILFSQSERSTPAKVAPAKPKIEGESSKTEMEKEH
CaLPAAT3
SEQ ID NO: 76
MTIASAAVVFLFGILLFTSGLIINLFQAFCSVLVWPLSKNAYRRINRVF
AEFLPLEFLWLFHWWAGAKLKLFTDPETFRLMGKEHALVIINHKIELDW
MVGWVLGQHLGCLGSILSVAKKSTKFLPVFGWSLWFSEYLFLERNWAKD
KKTLKSHIERLKDYPLPFWLIIFVEGTRFTRTKLLAAQQYAASAGLPVP
TRNVLIPHTKGFVSSVSHMRSFVPAIYDVTVAFPKSPPPTMLKLFEGHF
VELHVHIKRHAMKDLPESEDAVAQWCRDKFVEKDALLDKHNAEDTFSGQ
EVHHVGRPIKSLLVVISWVVVIIFGALKFLQWSSLLSSWKGIAFSVIGL
GTVALLMQILILSSQAERSIPAKETPANLKTELSSSKKVTNKEN
SalLPAAT1
SEQ ID NO: 77
MAIGAAAIVVPLGLLFMLSGLMVNLIQAICFILVRPLSKNMYRRVNRVV
VELLWLELIWLIDWWGGVKVDVYADSETFQSLGKEHALVVSNHKSDIDW
LVGWVLAQRSGCLGSTLAVMKKSSKFLPVIGWSMWFSEYVFLERSWAKD
ESTLKSGLQRLKDFPRPFWLALFVEGTRFTQAKLLAAQEYAASTGLPIP
RNVLIPRTKGFVSAVSNMRSFVPAIYDVTVAIPKTQPSPTMLRIFNRQP
SVVHVRIKRHSMNQLPPTDEGVAQWCKDIFVAKDALLDRHLAEGKFDEK
EFKRIRRPIKSLLVISSWSFLLLFGVFKFLKWSALLSTWKGVAVSTAVL
LLVTVVMYMFILFSQSERSSPRKVAPSGPENG
CleptLPAAT1
SEQ ID NO: 78
MAIPAAVVIFLFGLLFFSSGLIINLFQALCFVLIWPLSKNAYRRINRVF
AELLLSELLCLFDWWAGAKLKLFTDPETFRLMGKEHALVIINHMTELDW
MVGWVMGQHFGCLGSILSVAKKSTKFLPVLGWSMWFTEYLYIERSWDKD
KSTLKSHIERLKDYPLPFWLVIFAEGTRFTRTKLLAAQQYAASSGLPVP
RNVLIPRTKGFVSCVNHMRSFVPAVYDLTVAFPKTSPPPTLLNLFEGQS
VVLHVHIKRHAMKDLPESDDAVAQWCRDKFVEKDALLDKHNAEDTFSSQ
EVHHTGSRPIKSLLVVISWVVVITFGALKFLQWSSWKGKAFSVIGLGIV
TLLMHMLILSSQAERSKPAKVTQAKLKTELSISKKVTDKEN
ClopLPAAT1
SEQ ID NO: 79
MAIAAAAVIFLFGLLFFASGLIINLFQALCFVLIRPLSKNAYRRINRVF
AELLLSELLCLFDWWAGAKLKLFTDPETLRLMGKEHALIIINHMTELDW
MVGWVMGQHFGCLGSIISVAKKSTKFLPVLGWSMWFSEYLYLERSWAKD
KSTLKSHIERLKDYPLPFWLVIFVEGTRFTRTKLLAAQEYAASSGLPVP
RNVLIPRTKGFVSCVNHMRSFVPAVYDVTVAFPKTSPQPTLLNLFEGRS
IVLHVHIKRHAMKDLPESDDAVAQWCRDKFVEKDALLDKHNAEDTFSGQ
EVHHTGRRPIKSLLVVMSWVVVTTFGALKFLQWSSWKGKAFSVIGLGIV
TLLMHVLILSSQAERSNPAKVVQAELNTELSISKKVTNKGN
CcrasLPAAT1a
SEQ ID NO: 80
MAIPAAAVIFLFGLIFFASGLIINLFQALCFVLIWPLWKNAYRRINRVF
AELLLSELLCLFDWWAGAKLKLFTDPETFRLMGKEHALVIINHMTELDW
MVGWVMGQHFGCLGSILSVAKKSTKFLPVLGWSMWFTEYLYIERSWDKD
KSTLKSHIERLKDYPLPFWLVIFAEGTRFTRTKLLAAQQYAASSGLPVP
RNVLIPRTKGFVSSVSHMRSFVPAIYDVTVAIPKTSPPPTLIRMFKGQS
SVLHVHLKRHVMKDLPESDDAVAQWCRDIFVEKDALLDKHNAEDTFSGQ
ELQDTGRPIKSLLVVISWAVLEVFGAVKFLQWSSLLSSWKGLAFSGIGL
GIITLLMHILILFSQSERSTPAKVAPAKAK
CcrasLPAAT1b
SEQ ID NO: 81
MAIPAAAVIFLFGLIFFASGLIINLFQALCFVLIWPLWKNAYRRINRVF
AELLLSELLCLFDWWAGAKLKLFTDPETFRLMGKEHALVIINHMTELDW
MVGWVMGQHFGCLGSILSVAKKSTKFLPVLGWSMWFTEYLYIERSWDKD
GKSTLKSHIERLKDYPLPFWLVIFAETRFTRTKLLAAQQYAASSGLPVP
RNVLIPRTKGFVSSVSHMRSFVPAIYDVTVAIPKTSPPPTLIRMFKGQS
SVLHVHLKRHVMKDLPESDDAVAQWCRDIFVEKDALLDKHNAEDTFSGQ
ELQDTGRPIKSLLVRCFLVLSLIYLNGIILKLCGLCLQVVISWAVLEVF
GAVKFLQWSSLLSSWKGLAFSGIGLGIITLLMHILILFSQSERSTPAKV
APAKAK
CcrasLPAAT1c
SEQ ID NO: 82
MAIPAAAVIFLFGLIFFASGLIINLFQALCFVLIWPLWKNAYRRINRVF
AELLLSELLCLFDWWAGAKLKLFTDPETFRLMGKEHALVIINHMTELDW
MVGWVMGQHFGCLGSILSVAKKSTKFLPVLGWSMWFTEYLYIERSWDKD
KSTLKSHIERLKDYPLPFWLVIFAEGTRFTRTKLLAAQQYAASSGLPVP
RNVLIPRTKGFVSSVSHMRSFVPAIYDVTVAIPKTSPPPTLIRMFKGQS
SVLHVHLKRHVMKDLPESDDAVAQWCRDIFVEKDALLDKHNAEDTFSGQ
ELQDTGRPIKSLLVVISWAVLEVFGAVKFLQWSSLLSSWKGLAFSGIGL
GIITLLMHILILFSQSERSTPAKVAPAKAKMEGESSKTEMEMEK
CcrasLPAAT1d
SEQ ID NO: 83
MAIPAAAVIFLFGLIFFASGLIINLFQALCFVLIWPLWKNAYRRINRVF
AELLLSELLCLFDWWAGAKLKLFTDPETFRLMGKEHALVIINHMTELDW
MVGWVMGQHFGCLGSILSVAKKSTKFLPVLGWSMWFTEYLYIERSWDKD
KSTLKSHIERLKDYPLPFWLVIFAEGTRFTRTKLLAAQQYAASSGLPVP
RNVLIPRTKGFVSSVSHMRSFVPAIYDVTVAIPKTSPPPTLIRMFKGQS
SVLHVHLKRHVMKDLPESDDAVAQWCRDIFVEKDALLDKHNAEDTFSGQ
ELQDTGRPIKSLLVRCFLVLSLIYLNGIILKLCGLCLQVVISWAVLEVF
GAVKFLQWSSLLSSWKGLAFSGIGLGIITLLMHILILFSQSERSTPAKV
APAKAKMEGESSKTEMEMEK
CkoeLPAAT1
SEQ ID NO: 84
MAIAAAPVIFLFGLLFFASGLIINLFQAICFVLIWPLSKNAYRRINRVF
AELLLSELLCLFDWWAGAKLKLFTDPETFRLMGKEHALVITNHKIDLDW
MIGWILGQHFGCLGSVISIAKKSTKFLPIFGWSLWFSEYLFLERNWAKD
KRTLKSHIERMKDYPLPLWLILFVEGTRFTRTKLLAAQQYAASSGLPVP
RNVLIPHTKGFVSSVSHMRSFVPAIYDVTVAIPKTSPPPTLIRMFKGQS
SVLHVHLKRHLMKDLPESDDAVAQWCRDIFVEKDALLDKHNAEDTFSGQ
ELQETGRPIKSLLVVISWAVLEVYGAVKFLQWSSLLSSWKGLAFSGIGL
GLITLLMHILILFSQSERSTPAKVAPAKPKKEGESSKTEMEKEK
CkoeLPAAT2
SEQ ID NO: 85
MHVLLEMVTFRFSSFFVFDNVQALCFVLIWPLSKSAYRKINRVFAELLL
SELLCLFDWWAGAKLKLFTDPETFRLMGKEHALVITNHKIDLDWMIGWI
LGQHFGCLGSVISIAKKSTKFLPIFGWSLWFSEYLFLERNWAKDKRTLK
SHIERMKDYPLPLWLILFVEGTRFTRTKLLAAQQYAASSGLPVPRNVLI
PHTKGFVSSVSHMRSFVPAVYDVTVAFPKTSPPPTMLSLFEGQSVVLHV
THIKRHAMKDLPDSDDAVAQWCRDKFVEKDALLDKHNAEDFSGQEVHHV
GRPIKSLLVVISWMVVIIFGALKFLQWSSLLSSWKGKAFSAIGLGIATL
LMHVLVVFSQADRSNPAKVPPAKLNTELSSSKKVTNKEN

### Example 5: Expression of LPAATs to Improve Sn-2 Selectivity in Prototheca moriformis

In the example we disclose genetically engineered Prototheca moriformis strains in which we have modified fatty acid and triacylglycerol biosynthesis to maximize the accumulation of Stearoyl-Oleoyl-Stearoyl (SOS) TAGs, and minimize the production of trisaturated TAGs. Oils from these strains resemble plant seed oils known as “structuring fats”, which have high proportions of Saturated-Oleate-Saturated TAGs and low levels of trisaturates. These structuring fats (often called “butters”) are generally solid at room temperature but melt sharply between 35-40° C.

Strains with high SOS and low trisaturates were obtained by three successive transformations, beginning with S5100, a classically improved derivative of S376 (improved to increase lipid titer), a wild type isolate of Prototheca moriformis. S5100 was transformed with a construct to which increased expression of PmKASII-1 and ablated the SAD2-1 allele. The resultant strain, S5780, produced oil with increased C18:0 and lower C16:0 content relative to S5100. S5780 was prepared according to the methods disclosed in co-owned application WO2013/158938 and as described below. C18:0 levels were increased further by transformation of S5780 with a construct overexpressing the C18:0-specific FATA1 thioesterase gene from Garcinia mangostana (GarmFATA1), generating strain S6573. S6573 was disclosed in co-owned application WO2015/051319. Finally, accumulation of trisaturated TAGS was reduced by expression of genes encoding LPAATs from Brassica napus, Theobroma cacao, Garcinia hombororiana or Garcinia indica in S6573 as described below.

### Construct Used for SAD2 Knockout and PmKASII-1 Overexpression in S5100 to Produce S5780

The sequence of the transforming DNA from the SAD2-1 ablation, PmKASII over-expression construct, pSZ2624, is shown below. The construct is written as: pSZ2624: SAD2-1vD::PmKASII-1tp_PmKASII-1_FLAG-CvNR:CpACT-AtTHIC-CpEF1a::SAD2-1vE Relevant restriction sites are indicated in lowercase, bold, and are from 5′-3′ PmeI, SpeI, AscI, ClaI, SacI, AvrII, EcoRV, AflII, KpnI, XbaI, MfeI, BamHI, BspQI and PmeI. Underlined sequences at the 5′ and 3′ flanks of the construct represent genomic DNA from P. moriformis that enable targeted integration of the transforming DNA via homologous recombination at the SAD2-1 locus. The SAD2-1 5′ integration flank contained the endogeneous SAD2-1 promoter, enabling the in situ activation of the PmKASII gene. Proceeding in the 5′ to 3′ direction, the region encoding the PmKASII plastid targeting sequence is indicated by lowercase, underlined italics. The sequence that encodes the mature PmKASII polypeptide is indicated with lowercase italics, while a 3×FLAG epitope encoding sequence is in bold italics. The initiator ATG and terminator TGA for PmKASII-FLAG are indicated by uppercase italics. The 3′ UTR of the Chlorella vulgaris nitrate reductase (CvNR) gene is indicated by small capitals. Two spacer regions are represented by lowercase text. The CpACT promoter driving the expression of the AtTHIC gene (encoding 4-amino-5-hydroxymethyl-2-methylpyrimidine synthase activity, thereby permitting the strain to grow in the absence of exogeneous thiamine) is indicated by lowercase, boxed text. The initiator ATG and terminator TGA for AtTHIC are indicated by uppercase italics, while the coding region is indicated with lowercase italics. The 3′ UTR of the Chlorella protothecoides EF1a (CpEF1a) gene is indicated by small capitals. The use of THIC as a selection marker was described in co-owned applications WO2011/150410 and WO2013/150411.

pSZ2624 Nucleotide sequence of the transforming
DNA
SEQ ID NO: 86
gtttaaacGCCGGTCACCACCCGCATGCTCGTACTACAGCGCACGCACC
GCTTCGTGATCCACCGGGTGAACGTAGTCCTCGACGGAAACATCTGGTT
CGGGCCTCCTGCTTGCACTCCCGCCCATGCCGACAACCTTTCTGCTGTT
ACCACGACCCACAATGCAACGCGACACGACCGTGTGGGACTGATCGGTT
CACTGCACCTGCATGCAATTGTCACAAGCGCTTACTCCAATTGTATTCG
TTTGTTTTCTGGGAGCAGTTGCTCGACCGCCCGCGTCCCGCAGGCAGCG
ATGACGTGTGCGTGGCCTGGGTGTTTCGTCGAAAGGCCAGCAACCCTAA
ATCGCAGGCGATCCGGAGATTGGGATCTGATCCGAGTTTGGACCAGATC
CGCCCCGATGCGGCACGGGAACTGCATCGACTCGGCGCGGAACCCAGCT
TTCGTAAATGCCAGATTGGTGTCCGATACCTGGATTTGCCATCAGCGAA
ACAAGACTTCAGCAGCGAGCGTATTTGGCGGGCGTGCTACCAGGGTTGC
ATACATTGCCCATTTCTGTCTGGACCGCTTTACTGGCGCAGAGGGTGAG
TTGATGGGGTTGGCAGGCATCGAAACGCGCGTGCATGGTGTGCGTGTCT
GTTTTCGGCTGCACGAATTCAATAGTCGGATGGGCGACGGTAGAATTGG
GTGTGGCGCTCGCGTGCATGCCTCGCCCCGTCGGGTGTCATGACCGGGA
CTGGAATCCCCCCTCGCGACCATCTTGCTAACGCTCCCGACTCTCCCGA
CCGCGCGCAGGATAGACTCTTGTTCAACCAATCGACAactagtATGcag
accgcccaccagcgcccccccaccgagggccactgatcggcgcccgcct
gcccaccgcctcccgccgcgccgtgcgccgcgcctggtcccgcatcgcc
cgcgggcgcgccgccgccgccgccgacgccaaccccgcccgccccgagc
gccgcgtggtgatcaccggccagggcgtggtgacctccctgggccagac
catcgagcagactactcctccctgctggagggcgtgtccggcatctccc
agatccagaagacgacaccaccggctacaccaccaccatcgccggcgag
atcaagtccctgcagctggacccctacgtgcccaagcgctgggccaagc
gcgtggacgacgtgatcaagtacgtgtacatcgccggcaagcaggccct
ggagtccgccggcctgcccatcgaggccgccggcctggccggcgccggc
ctggaccccgccctgtgcggcgtgctgatcggcaccgccatggccggca
tgacctccacgccgccggcgtggaggccctgacccgcggcggcgtgcgc
aagatgaaccccactgcatccccactccatctccaacatgggcggcgcc
atgctggccatggacatcggatcatgggccccaactactccatctccac
cgcctgcgccaccggcaactactgcatcctgggcgccgccgaccacatc
cgccgcggcgacgccaacgtgatgctggccggcggcgccgacgccgcca
tcatcccctccggcatcggcggcttcatcgcctgcaaggccctgtccaa
gcgcaacgacgagcccgagcgcgcctcccgcccctgggacgccgaccgc
gacggatcgtgatgggcgagggcgccggcgtgctggtgctggaggagct
ggagcacgccaagcgccgcggcgccaccatcctggccgagctggtgggc
ggcgccgccacctccgacgcccaccacatgaccgagcccgacccccagg
gccgcggcgtgcgcctgtgcctggagcgcgccctggagcgcgcccgcct
ggcccccgagcgcgtgggctacgtgaacgcccacggcacctccaccccc
gccggcgacgtggccgagtaccgcgccatccgcgccgtgatcccccagg
actccctgcgcatcaactccaccaagtccatgatcggccacctgctggg
cggcgccggcgccgtggaggccgtggccgccatccaggccctgcgcacc
ggctggctgcaccccaacctgaacctggagaaccccgcccccggcgtgg
accccgtggtgctggtgggcccccgcaaggagcgcgccgaggacctgga
cgtggtgctgtccaactccttcggcttcggcggccacaactcctgcgtg
atcttccgcaagtacgacgag

[symbol]

[symbol]

[symbol]

[symbol]

[symbol]

[symbol]

TGAatcgatAGATCTCTTAAGGCAGCAGCAGCTCGGATAGTATCGACA
CACTCTGGACGCTGGTCGTGTGATGGACTGTTGCCGCCACACTTGCTGC
CTTGACCTGTGAATATCCCTGCCGCTTTTATCAAACAGCCTCAGTGTGT
TTGATCTTGTGTGTACGCGCTTTTGCGAGTTGCTAGCTGCTTGTGCTAT
TTGCGAATACCACCCCCAGCATCCCCTTCCCTCGTTTCATATCGCTTGC
ATCCCAACCGCAACTTATCTACGCTGTCCTGCTATCCCTCAGCGCTGCT
CCTGCTCCTGCTCACTGCCCCTCGCACAGCCTTGGTTTGGGCTCCGCCT
GTATTCTCCTGGTACTGCAACCTGTAAACCAGCACTGCAATGCTGATGC
ACGGGAAGTAGTGGGATGGGAACACAAATGGAAAGCTTAATTAAgagct
ccgcgtctcgaacagagcgcgcagaggaacgctgaaggtctcgcctctg
tcgcacctcagcgcggcatacaccacaataaccacctgacgaatgcgct
tggttcttcgtccattagcgaagcgtccggttcacacacgtgccacgtt
ggcgaggtggcaggtgacaatgatcggtggagctgatggtcgaaacgtt
cacagcctaggtgatatccatcttaaggatctaagtaagattcgaagcg
ctcgaccgtgccggacggactgcagccccatgtcgtagtgaccgccaat
gtaagtgggctggcgtaccctgtacgtgagtcaacgtcactgcacgcgc
accaccctctcgaccggcaggaccaggcatcgcgagatacagcgcgagc
cagacacggagtgccgagctatgcgcacgctccaactaggtaccagttt
aggtccagcgtccgtggggggggacgggctgggagcttgggccgggaag
ggcaagacgatgcagtccctctggggagtcacagccgactgtglgtgtt
gcactgtgcggcccgcagcactcacacgcaaaatgcctggccgacaggc
aggccctgtccagtgcaacatccacggtccctctcatcaggctcacctt
gctcattgacataacggaatgcgtaccgctctttcagatctgtccatcc
agagaggggagcaggctccccaccgacgctgtcaaacttgcttcctgcc
caaccgaaaacattattgtttgagggggggggggggggggcagattgca
tggcgggatatctcgtgaggaacatcactgggacactgtggaacacagt
gagtgcagtatgcagagcatgtatgctaggggtcagcgcaggaaggggg
cctttcccagtctcccatgccactgcaccgtatccacgactcaccagga
ccagcttcttgatcggcttccgctcccgtggacaccagtglgtagcctc
tggactccaggtatgcgtgcaccgcaaaggccagccgatcgtgccgatt
cctgggtggaggatatgagtcagccaacttggggctcagagtgcacact
ggggcacgatacgaaacaacatctacaccgtgtcctccatgctgacaca
ccacagcttcgctccacctgaatgtgggcgcatgggcccgaatcacagc
caatgtcgctgctgccataatgtgatccagaccctctccgcccagatgc
cgagcggatcgtgggcgctgaatagattcctgtttcgatcactgtttgg
gtcctttccttttcgtctcggatgcgcgtctcgaaacaggctgcgtcgg
gctttcggatcccttttgctccctccgtcaccatcctgcgcgcgggcaa
gttgcttgaccctgggctgataccagggttggagggtattaccgcgtca
ggccattcccagcccggattcaattcaaagtctgggccaccaccctccg
ccgctctgtctgatcactccacattcgtgcatacactacgttcaagtcc
tgatccaggcgtgtctcgggacaaggtgtgcttgagtttgaatctcaag
gacccactccagcacagctgctggttgaccccgccctcgcaatctagaA
TGgccgcgtccgtccactgcaccctgatgtccgtggtctgcaacaacaa
gaaccactccgcccgccccaagctgcccaactcctccctgctgcccgga
tcgacgtggtggtccaggccgcggccacccgatcaagaaggagacgacg
accacccgcgccacgctgacgacgacccccccacgaccaactccgagcg
cgccaagcagcgcaagcacaccatcgacccctcctcccccgacaccagc
ccatcccctccacgaggagtgatccccaagtccacgaaggagcacaagg
aggtggtgcacgaggagtccggccacgtcctgaaggtgcccaccgccgc
gtgcacctgtccggcggcgagcccgccacgacaactacgacacgtccgg
cccccagaacgtcaacgcccacatcggcctggcgaagctgcgcaaggag
tggatcgaccgccgcgagaagctgggcacgccccgctacacgcagatgt
actacgcgaagcagggcatcatcacggaggagatgctgtactgcgcgac
gcgcgagaagctggaccccgagacgtccgctccgaggtcgcgcggggcc
gcgccatcatcccctccaacaagaagcacctggagctggagcccatgat
cgtgggccgcaagacctggtgaaggtgaacgcgaacatcggcaactccg
ccgtggcctcctccatcgaggaggaggtctacaaggtgcagtgggccac
catgtggggcgccgacaccatcatggacctgtccacgggccgccacatc
cacgagacgcgcgagtggatcctgcgcaactccgcggtccccgtgggca
ccgtccccatctaccaggcgctggagaaggtggacggcatcgcggagaa
cctgaactgggaggtgaccgcgagacgctgatcgagcaggccgagcagg
gcgtggactacttcacgatccacgcgggcgtgctgctgcgctacatccc
cctgaccgccaagcgcctgacgggcatcgtgtcccgcggcggctccatc
cacgcgaagtggtgcctggcctaccacaaggagaacttcgcctacgagc
actgggacgacatcctggacatctgcaaccagtacgacgtcgccctgtc
catcggcgacggcctgcgccccggctccatctacgacgccaacgacacg
gcccagacgccgagctgctgacccagggcgagctgacgcgccgcgcgtg
ggagaaggacgtgcaggtgatgaacgagggccccggccacgtgcccatg
cacaagatccccgagaacatgcagaagcagctggagtggtgcaacgagg
cgcccactacaccctgggccccctgacgaccgacatcgcgcccggctac
gaccacatcacctccgccatcggcgcggccaacatcggcgccctgggca
ccgccctgctgtgctacgtgacgcccaaggagcacctgggcctgcccaa
ccgcgacgacgtgaaggcgggcgtcatcgcctacaagatcgccgcccac
gcggccgacctggccaagcagcacccccacgcccaggcgtgggacgacg
cgctgtccaaggcgcgatcgagaccgctggatggaccagacgcgctgtc
cctggaccccatgacggcgatgtccaccacgacgagacgctgcccgcgg
acggcgcgaaggtcgcccacactgctccatgtgcggccccaagactgct
ccatgaagatcacggaggacatccgcaagtacgccgaggagaacggcta
cggctccgccgaggaggccatccgccagggcatggacgccatgtccgag
gagacaacatcgccaagaagacgatctccggcgagcagcacggcgaggt
cggcggcgagatctacctgcccgagtcctacgtcaaggccgcgcagaag
TGAcaattgACGGAGCGTCGTGCGGGAGGGAGTGTGCCGAGCGGGGAGT
CCCGGTCTGTGCGAGGCCCGGCAGCTGACGCTGGCGAGCCGTACGCCCC
GAGGGTCCCCCTCCCCTGCACCCTCTTCCCCTTCCCTCTGACGGCCGCG
CCTGTTCTTGCATGTTCAGCGACggatccTAGGGAGCGACGAGTGTGCG
TGCGGGGCTGGCGGGAGTGGGACGCCCTCCTCGCTCCTCTCTGTTCTGA
ACGGAACAATCGGCCACCCCGCGCTACGCGCCACGCATCGAGCAACGAA
GAAAACCCCCCGATGATAGGTTGCGGTGGCTGCCGGGATATAGATCCGG
CCGCACATCAAAGGGCCCCTCCGCCAGAGAAGAAGCTCCTTTCCCAGCA
GACTCCTTCTGCTGCCAAAACACTTCTCTGTCCACAGCAACACCAAAGG
ATGAACAGATCAACTTGCGTCTCCGCGTAGCTTCCTCGGCTAGCGTGCT
TGCAACAGGTCCCTGCACTATTATCTTCCTGCTTTCCTCTGAATTATGC
GGCAGGCGAGCGCTCGCTCTGGCGAGCGCTCCTTCGCGCCGCCCTCGCT
GATCGAGTGTACAGTCAATGAATGGTCCTGGGCGAAGAACGAGGGAATT
TGTGGGTAAAACAAGCATCGTCTCTCAGGCCCCGGCGCAGTGGCCGTTA
AAGTCCAAGACCGTGACCAGGCAGCGCAGCGCGTCCGTGTGCGGGCCCT
GCCTGGCGGCTCGGCGTGCCAGGCTCGAGAGCAGCTCCCTCAGGTCGCC
TTGGACGGCCTCTGCGAGGCCGGTGAGGGCCTGCAGGAGCGCCTCGAGC
GTGGCAGTGGCGGTCGTATCCGGGTCGCCGGTCACCGCCTGCGACTCGC
CATCCgaagagcgtttaaac

Construct D1683 (pSZ2624), was transformed into S5100. Primary transformants were clonally purified and grown under standard lipid production conditions at pH 5. Integration of pSZ2624 at the SAD2-1 locus was verified by DNA blot analysis. The fatty acid profiles and lipid titers of lead strains were assayed in 50-mL shake flasks (Table 8). Simultaneous ablation of SAD2-1 and over-expression of PmKASII (driven in situ by the SAD2-1 promoter) resulted in C18:0 levels up to 26.1%. C16:0 accumulation was reduced from 15.3% in S5100 to <6% the strains derived from D1683, demonstrating that PmKASII-1 over-expression promoted the elongation of C16:0 to C18:0. S5780 was chosen for further development as it had the highest lipid titer relative to the S5100 parent.

TABLE 8
Fatty acid profiles of SAD2-1 ablation, PmKASII-1 overexpression
strains derived from D1683-1, compared to the S5100 parent.

| Primary | | | S5100; T531; D1683.1 | | | | |
|---|---|---|---|---|---|---|---|
| Strain | | S5100 | S5780 | S5781 | S5782 | S5783 | S5784 |
| Fatty Acid | C14:0 | 0.7 | 0.7 | 0.8 | 0.7 | 0.7 | 0.7 |
| Area % | C16:0 | 15.3 | 5.9 | 6.0 | 6.0 | 5.8 | 5.8 |
| | C16:1 | 0.5 | 0.1 | 0.0 | 0.1 | 0.0 | 0.0 |
| | C18:0 | 4.0 | 25.6 | 26.1 | 26.0 | 25.0 | 25.3 |
| | C18:1 | 71.0 | 55.7 | 54.5 | 54.6 | 56.3 | 55.6 |
| | C18:2 | 7.3 | 8.0 | 8.5 | 8.5 | 8.1 | 8.4 |
| | C18:3 α | 0.5 | 0.7 | 0.8 | 0.8 | 0.7 | 0.7 |
| | C20:0 | 0.3 | 1.8 | 1.9 | 1.8 | 1.8 | 1.8 |
| | C20:1 | 0.2 | 0.6 | 0.6 | 0.6 | 0.7 | 0.7 |
| | C22:0 | 0.1 | 0.2 | 0.3 | 0.3 | 0.3 | 0.2 |
| | C24:0 | 0.1 | 0.4 | 0.4 | 0.4 | 0.4 | 0.4 |
| | saturates | 20.6 | 34.7 | 35.6 | 35.4 | 34.1 | 34.5 |

We disclose additional methods of elevating C18:0 levels that can be used in conjunction with SAD2 knockout and KASII over-expression. Previously we described acyl-ACP thioesterases from Brassica napus (BnFATA) (Co-owned application WO2012/106560), Garcinia mangostana (GarmFATA1) (Co-owned application WO2015/051319) and Theobroma cacao (TcFATA) (Co-owned application WO2013/158938) with specificity towards cleavage of C18:0-ACP, and we observed that average C18:0 levels were higher in strains in which we replaced the native BnFATA transit peptide with the Chlorella protothecoides SAD1 transit peptide (CpSAD1tp). A DNA construct was made for expression of a chimeric gene encoding CpSAD1tp fused to the predicted GarmFATA1 mature polypeptide and a FLAG tag sequence.

The sequence of the transforming DNA from the GarmFATA1 expression construct pSZ3204 is shown below. The construct is written as pSZ3204: 6SA::CrTUB2-ScSUC2-CvNR:PmSAD2-2-CpSAD1tp_GarmFATA1_FLAG-CvNR::6SB. Relevant restriction sites are indicated in lowercase, bold, and are from 5′-3′ BspQI, KpnI, XbaI, MfeI, BamHI, AvrII, EcoRV, SpeI, AscI, ClaI, AflII, SacI and BspQI. Underlined sequences at the 5′ and 3′ flanks of the construct represent genomic DNA from P. moriformis that enable targeted integration of the transforming DNA via homologous recombination at the 6S locus. Proceeding in the 5′ to 3′ direction, the CrTUB2 promoter driving the expression of Saccharomyces cerevisiae SUC2 (ScSUC2) gene, enabling strains to utilize exogeneous sucrose, is indicated by lowercase, boxed text. The initiator ATG and terminator TGA of ScSUC2 are indicated by uppercase italics, while the coding region is represented by lowercase italics. The 3′ UTR of the CvNR gene is indicated by small capitals. A spacer region is represented by lowercase text. The P. moriformis SAD2-2 (PmSAD2-2) promoter driving the expression of the chimeric CpSAD1tp_GarmFATA1_FLAG gene is indicated by lowercase, boxed text. The initiator ATG and terminator TGA are indicated by uppercase italics; the sequence encoding CpSAD1tp is represented by lowercase, underlined italics; the sequence encoding the GarmFATA1 mature polypeptide is indicated by lowercase italics; and the 3×FLAG epitope tag is represented by uppercase, bold italics. A second CvNR 3′ UTR is indicated by small capitals.

pSZ3204
SEQ ID NO: 87
gctcttcGCCGCCGCCACTCCTGCTCGAGCGCGCCCGCGCGTGCGCCGC
CAGCGCCTTGGCCTTTTCGCCGCGCTCGTGCGCGTCGCTGATGTCCATC
ACCAGGTCCATGAGGTCTGCCTTGCGCCGGCTGAGCCACTGCTTCGTCC
GGGCGGCCAAGAGGAGCATGAGGGAGGACTCCTGGTCCAGGGTCCTGAC
GTGGTCGCGGCTCTGGGAGCGGGCCAGCATCATCTGGCTCTGCCGCACC
GAGGCCGCCTCCAACTGGTCCTCCAGCAGCCGCAGTCGCCGCCGACCCT
GGCAGAGGAAGACAGGTGAGGGGGGTATGAATTGTACAGAACAACCACG
AGCCTTGTCTAGGCAGAATCCCTACCAGTCATGGCTTTACCTGGATGAC
GGCCTGCGAACAGCTGTCCAGCGACCCTCGCTGCCGCCGCTTCTCCCGC
ACGCTTCTTTCCAGCACCGTGATGGCGCGAGCCAGCGCCGCACGCTGGC
GCTGCGCTTCGCCGATCTGAGGACAGTCGGGGAACTCTGATCAGTCTAA
ACCCCCTTGCGCGTTAGTGTTGCCATCCTTTGCAGACCGGTGAGAGCCG
ACTTGTTGTGCGCCACCCCCCACACCACCTCCTCCCAGACCAATTCTGT
CACCTTTTTGGCGAAGGCATCGGCCTCGGCCTGCAGAGAGGACAGCAGT
GCCCAGCCGCTGGGGGTTGGCGGATGCACGCTCAggtaccattcttgcg
ctatgacacttccagcaaaaggtagggcgggctgcgagacggcttcccg
gcgctgcatgcaacaccgatgatgcttcgaccccccgaagctccttcgg
ggctgcatgggcgctccgatgccgctccagggcgagcgctgtttaaata
gccaggcccccgattgcaaagacattatagcgagctaccaaagccatat
tcaaacacctagatcactaccacttctacacaggccactcgagcttgtg
atcgcactccgctaagggggcgcctcttcctcttcgtttcagtcacaac
ccgcaaactctagaatatcaATGctgctgcaggccttcctgttcctgct
ggccggcttcgccgccaagatcagcgcctccatgacgaacgagacgtcc
gaccgccccctggtgcacttcacccccaacaagggctggatgaacgacc
ccaacggcctgtggtacgacgagaaggacgccaagtggcacctgtactt
ccagtacaacccgaacgacaccgtctgggggacgcccttgttctggggc
cacgccacgtccgacgacctgaccaactgggaggaccagcccatcgcca
tcgccccgaagcgcaacgactccggcgccttctccggctccatggtggt
ggactacaacaacacctccggcttcttcaacgacaccatcgacccgcgc
cagcgctgcgtggccatctggacctacaacaccccggagtccgaggagc
agtacatctcctacagcctggacggcggctacaccttcaccgagtacca
gaagaaccccgtgctggccgccaactccacccagttccgcgacccgaag
gtcttctggtacgagccctcccagaagtggatcatgaccgcggccaagt
cccaggactacaagatcgagatctactcctccgacgacctgaagtcctg
gaagctggagtccgcgttcgccaacgagggcttcctcggctaccagtac
gagtgccccggcctgatcgaggtccccaccgagcaggaccccagcaagt
cctactgggtgatgttcatctccatcaaccccggcgccccggccggcgg
ctccttcaaccagtacttcgtcggcagcttcaacggcacccacttcgag
gccttcgacaaccagtcccgcgtggtggacttcggcaaggactactacg
ccctgcagaccttcttcaacaccgacccgacctacgggagcgccctggg
catcgcgtgggcctccaactgggagtactccgccttcgtgcccaccaac
ccctggcgctcctccatgtccctcgtgcgcaagttctccctcaacaccg
agtaccaggccaacccggagacggagctgatcaacctgaaggccgagcc
gatcctgaacatcagcaacgccggcccctggagccggttcgccaccaac
accacgttgacgaaggccaacagctacaacgtcgacctgtccaacagca
ccggcaccctggagttcgagctggtgtacgccgtcaacaccacccagac
gatctccaagtccgtgttcgcggacctctccctctggttcaagggcctg
gaggaccccgaggagtacctccgcatgggcttcgaggtgtccgcgtcct
ccttcttcctggaccgcgggaacagcaaggtgaagttcgtgaaggagaa
cccctacttcaccaaccgcatgagcgtgaacaaccagcccttcaagagc
gagaacgacctgtcctactacaaggtgtacggcttgctggaccagaaca
tcctggagctgtacttcaacgacggcgacgtcgtgtccaccaacaccta
cttcatgaccaccgggaacgccctgggctccgtgaacatgacgacgggg
gtggacaacctgttctacatcgacaagttccaggtgcgcgaggtcaagT
GAcaattgGCAGCAGCAGCTCGGATAGTATCGACACACTCTGGACGCTG
GTCGTGTGATGGACTGTTGCCGCCACACTTGCTGCCTTGACCTGTGAAT
ATCCCTGCCGCTTTTATCAAACAGCCTCAGTGTGTTTGATCTTGTGTGT
ACGCGCTTTTGCGAGTTGCTAGCTGCTTGTGCTATTTGCGAATACCACC
CCCAGCATCCCCTTCCCTCGTTTCATATCGCTTGCATCCCAACCGCAAC
TTATCTACGCTGTCCTGCTATCCCTCAGCGCTGCTCCTGCTCCTGCTCA
CTGCCCCTCGCACAGCCTTGGTTTGGGCTCCGCCTGTATTCTCCTGGTA
CTGCAACCTGTAAACCAGCACTGCAATGCTGATGCACGGGAAGTAGTGG
GATGGGAACACAAATGGAggatcccgcgtctcgaacagagcgcgcagag
gaacgctgaaggtctcgcctctgtcgcacctcagcgcggcatacaccac
aataaccacctgacgaatgcgcttggttcttcgtccattagcgaagcgt
ccggttcacacacgtgccacgttggcgaggtggcaggtgacaatgatcg
gtggagctgatggtcgaaacgttcacagcctagggatatcctgaagaat
gggaggcaggtgttgttgattatgagtgtgtaaaagaaaggggtagaga
gccgtcctcagatccgactactatgcaggtagccgctcgcccatgcccg
cctggctgaatattgatgcatgcccatcaaggcaggcaggcatactgtg
cacgcaccaagcccacaatcttccacaacacacagcatgtaccaacgca
cgcgtaaaagttggggtgctgccagtgcgtcatgccaggcatgatgtgc
tcctgcacatccgccatgatctcctccatcgtctcgggtgtttccggcg
cctggtccgggagccgttccgccagatacccagacgccacctccgacct
cacggggtacttttcgagcgtctgccggtagtcgacgatcgcgtccacc
atggagtagccgaggcgccggaactggcgtgacggagggaggagaggga
ggagagagaggggggggggggggggggatgattacacgccagtctcaca
acgcatgcaagacccgtttgattatgagtacaatcatgcactactagat
ggatgagcgccaggcataaggcacaccgacgttgatggcatgagcaact
cccgcatcatatttcctattgtcctcacgccaagccggtcaccatccgc
atgctcatattacagcgcacgcaccgcttcgtgatccaccgggtgaacg
tagtcctcgacggaaacatctggctcgggcctcgtgctggcactccctc
ccatgccgacaacctttctgctgtcaccacgacccacgatgcaacgcga
cacgacccggtgggactgatcggttcactgcacctgcatgcaattgtca
caagcgcatactccaatcgtatccgtttgatttctgtgaaaactcgctc
gaccgcccgcgtcccgcaggcagcgatgacgtgtgcgtgacctgggtgt
ttcgtcgaaaggccagcaaccccaaatcgcaggcgatccggagattggg
atctgatccgagcttggaccagatcccccacgatgcggcacgggaactg
catcgactcggcgcggaacccagcMcgtaaatgccagattggtgtccga
taccttgatttgccatcagcgaaacaagacttcagcagcgagcgtattt
ggcgggcgtgctaccagggttgcatacattgcccatttctgtctggacc
gctttaccggcgcagagggtgagttgatggggttggcaggcatcgaaac
gcgcgtgcatggtgtgtgtgtctgttttcggctgcacaatttcaatagt
cggatgggcgacggtagaattgggtgttgcgctcgcgtgcatgcctcgc
cccgtcgggtgtcatgaccgggactggaatcccccctcgcgaccctcct
gctaacgctcccgactctcccgcccgcgcgcaggatagactctagttca
accaatcgacaactagtATGgccaccgcatccactactcggcgacaatg
cccgctgcggcgacctgcgtcgctcggcgggctccgggccccggcgccc
agcgaggcccctccccgtgcgcgggcgcgccatccccccccgcatcatc
gtggtgtcctcctcctcctccaaggtgaaccccctgaagaccgaggccg
tggtgtcctccggcctggccgaccgcctgcgcctgggctccctgaccga
ggacggcctgtcctacaaggagaagacatcgtgcgctgctacgaggtgg
gcatcaacaagaccgccaccgtggagaccatcgccaacctgctgcagga
ggtgggctgcaaccacgcccagtccgtgggctactccaccggcggatac
caccacccccaccatgcgcaagctgcgcctgatctgggtgaccgcccgc
atgcacatcgagatctacaagtaccccgcctggtccgacgtggtggaga
tcgagtcctggggccagggcgagggcaagatcggcacccgccgcgactg
gatcctgcgcgactacgccaccggccaggtgatcggccgcgccacctcc
aagtgggtgatgatgaaccaggacacccgccgcctgcagaaggtggacg
tggacgtgcgcgacgagtacctggtgcactgcccccgcgagctgcgcct
ggccaccccgaggagaacaactcctccctgaagaagatctccaagctgg
aggacccctcccagtactccaagctgggcctggtgccccgccgcgccga
cctggacatgaaccagcacgtgaacaacgtgacctacatcggctgggtg
ctggagtccatgccccaggagatcatcgacacccacgagctgcagacca
tcaccctggactaccgccgcgagtgccagcacgacgacgtggtggactc
cctgacctcccccgagccctccgaggacgccgaggccgtgacaaccaca
acggcaccaacggctccgccaacgtgtccgccaacgaccacggctgccg
caacacctgcacctgctgcgcctgtccggcaacggcctggagatcaacc
gcggccgcaccgagtggcgcaagaagcccacccgc

[symbol]

[symbol]

[symbol]

[symbol]

[symbol]

[symbol]

TGAatcgatagatctcttaagGCAGCAGCAGCTCGGATA
GTATCGACACACTCTGGACGCTGGTCGTGTGATGGACTGTTGCCGCCAC
ACTTGCTGCCTTGACCTGTGAATATCCCTGCCGCTTTTATCAAACAGCC
TCAGTGTGTTTGATCTTGTGTGTACGCGCTTTTGCGAGTTGCTAGCTGC
TTGTGCTATTTGCGAATACCACCCCCAGCATCCCCTTCCCTCGTTTCAT
ATCGCTTGCATCCCAACCGCAACTTATCTACGCTGTCCTGCTATCCCTC
AGCGCTGCTCCTGCTCCTGCTCACTGCCCCTCGCACAGCCTTGGTTTGG
GCTCCGCCTGTATTCTCCTGGTACTGCAACCTGTAAACCAGCACTGCAA
TGCTGATGCACGGGAAGTAGTGGGATGGGAACACAAATGGAaagcttaa
ttaagagctcTTGTTTTCCAGAAGGAGTTGCTCCTTGAGCCTTTCATTC
TCAGCCTCGATAACCTCCAAAGCCGCTCTAATTGTGGAGGGGGTTCGAA
TTTAAAAGCTTGGAATGTTGGTTCGTGCGTCTGGAACAAGCCCAGACTT
GTTGCTCACTGGGAAAAGGACCATCAGCTCCAAAAAACTTGCCGCTCAA
CACCGCGTACCTCTGCTTTGCGCAATCTGCCCTGTTGAAATCGCCACCA
CATTCATATTGTGACGCTTGAGCAGTCTGTAATTGCCTCAGAATGTGGA
ATCATCTGCCCCCTGTGCGAGCCCATGCCAGGCATGTCGCGGGCGAGGA
CACCCGCCACTCGTACAGCAGACCATTATGCTACCTCACAATAGTTCAT
AACAGTGACCATATTTCTCGAAGCTCCCCAACGAGCACCTCCATGCTCT
GAGTGGCCACCCCCCGGCCCTGGTGCTTGCGGAGGGCAGGTCAACCGGC
ATGGGGCTACCGAAATCCCCGACCGGATCCCACCACCCCCGCGATGGGA
AGAATCTCTCCCCGGGATGTGGGCCCACCACCAGCACAACCTGCTGGCC
CAGGCGAGCGTCAAACCATACCACACAAATATCCTTGGCATCGGCCCTG
AATTCCTTCTGCCGCTCTGCTACCCGGTGCTTCTGTCCGAAGCAGGGGT
TGCTAGGGATCGCTCCGAGTCCGCAAACCCTTGTCGCGTGGCGGGGCTT
GTTCGAGCTTgaagagc

Construct D1940 (pSZ3204), was transformed into the S5780 parent strain. Primary transformants were clonally purified and grown under standard lipid production conditions at pH 5. Integration of pSZ3204 at the 6S locus was verified by DNA blot analysis. The fatty acid profiles and lipid titers of lead strains were assayed in 50-mL shake flasks (Table 9). Over-expression of GarmFATA1 (driven by the SAD2-2 promoter) resulted in C18:0 levels up to 54.3%. C16:0 levels were comparable in strains derived from D1940 and the S5780 parent. S6573 was chosen for further development as it had the highest lipid titer of the strains with >50% C18:0.

TABLE 9
Fatty acid profiles of GarmFATA1 overexpressing stable strains
derived from D1940 primary transformants.

| Primary | | | D1683.1 | D1940.19 | | | D1940.20 | D1940.23 | D1940.46 | D1940.5 |
|---|---|---|---|---|---|---|---|---|---|---|
| Strain | | S5100 | S5780 | S6571 | S6572 | S6573 | S6574 | S6575 | S6578 | S6580 |
| Fatty Acid Area % | C14:0 | 0.7 | 0.0 | 0.8 | 0.0 | 0.8 | 0.7 | 0.7 | 0.0 | 0.0 |
| | C16:0 | 18.0 | 5.9 | 6.3 | 6.6 | 6.3 | 5.0 | 5.1 | 5.0 | 5.3 |
| | C16:1 | 0.5 | 0.0 | 0.1 | 0.1 | 0.1 | 0.0 | 0.1 | 0.1 | 0.1 |
| | C18:0 | 3.9 | 29.0 | 52.7 | 54.3 | 53.7 | 43.1 | 46.0 | 45.4 | 47.9 |
| | C18:1 | 69.8 | 54.3 | 31.4 | 30.1 | 30.5 | 41.5 | 38.5 | 40.0 | 37.2 |
| | C18:2 | 5.9 | 6.4 | 5.7 | 5.8 | 5.6 | 6.3 | 6.2 | 6.1 | 6.2 |
| | C18:3 α | 0.5 | 0.7 | 0.6 | 0.6 | 0.6 | 0.6 | 0.5 | 0.6 | 0.5 |
| | C20:0 | 0.3 | 2.4 | 1.8 | 1.6 | 1.7 | 2.1 | 2.0 | 2.0 | 2.0 |
| | C20:1 | 0.1 | 0.6 | 0.1 | 0.1 | 0.1 | 0.2 | 0.1 | 0.1 | 0.1 |
| | C22:0 | 0.1 | 0.3 | 0.2 | 0.2 | 0.2 | 0.3 | 0.3 | 0.2 | 0.2 |
| | C24:0 | 0.0 | 0.0 | 0.0 | 0.0 | 0.0 | 0.0 | 0.0 | 0.0 | 0.0 |
| | saturates | 23.1 | 37.7 | 61.9 | 62.8 | 62.8 | 51.2 | 54.2 | 52.7 | 55.5 |

Lysophosphatidic acid acetyltransferase (LPAAT) enzymes are responsible for the transfer of acyl groups to the sn-2 position on the glycerol backbone. We disclose here that we can reduce the accumulation of excessive amounts of trisaturates in our high SOS strains by expressing heterologous LPAAT genes which were better than the endogenous acyltransferases at discriminating against saturated fatty acids. Expression of LPAT2 homologs from B. napus, T cacao, Garcinia hombroriana and Garcinia indica and their effect on the formation of trisaturated TAGs in the high-C18:0 S6573 strain is disclosed below.

The sequence of the transforming DNA from the BnLPAT2(Bn1.13) expression construct pSZ4198 is shown below The construct is written as pSZ4198: PLOOP::PmHXT1-ScarMEL1-CvNR:PmSAD2-2v2-BnLPAT2(Bn1.13)-CvNR::PLOOP. Relevant restriction sites are indicated in lowercase, bold, and are from 5′-3′ BspQI, KpnI, SpeI, SnaBI, EcoRI, SpeI, ClaI, BglII, AflII, HindIII, SacI and BspQI. Underlined sequences at the 5′ and 3′ flanks of the construct represent genomic DNA from P. moriformis that enable targeted integration of the transforming DNA via homologous recombination at the PLOOP locus. Proceeding in the 5′ to 3′ direction, the PmHXT1 promoter driving the expression of S. carlbergensis MEL1 (ScarMEL1) gene, enabling strains to utilize exogeneous melibiose, is indicated by lowercase, boxed text. The initiator ATG and terminator TGA of ScarMEL1 are indicated by uppercase italics, while the coding region is represented by lowercase italics. The 3′ UTR of the CvNR gene is indicated by small capitals. The P. moriformis SAD2-2v2 promoter driving the expression of the BnLPAT2(Bn1.13) gene is indicated by lowercase, boxed text. The initiator ATG and terminator TGA are indicated by uppercase italics; the sequence encoding BnLPAT2(Bn1.13) is represented by lowercase, underlined italics. A second CvNR 3′ UTR is indicated by small capitals. The Brassica napus LPAAT2(BN1.13) sequence is from Genbank accession GU045434.

SEQ ID NO: 88: Nucleotide sequence of the transforming DNA from pSZ4198
gctcttccgctAACGGAGGTCTGTCACCAAATGGACCCCGTCTATTGCGGGAAACCACG
GCGATGGCACGTTTCAAAACTTGATGAAATACAATATTCAGTATGTCGCGGGCGG
CGACGGCGGGGAGCTGATGTCGCGCTGGGTATTGCTTAATCGCCAGCTTCGCCCC
CGTCTTGGCGCGAGGCGTGAACAAGCCGACCGATGTGCACGAGCAAATCCTGAC
ACTAGAAGGGCTGACTCGCCCGGCACGGCTGAATTACACAGGCTTGCAAAAATA
CCAGAATTTGCACGCACCGTATTCGCGGTATTTTGTTGGACAGTGAATAGCGATG
CGGCAATGGCTTGTGGCGTTAGAAGGTGCGACGAAGGTGGTGCCACCACTGTGC
CAGCCAGTCCTGGCGGCTCCCAGGGCCCCGATCAAGAGCCAGGACATCCAAACT
ACCCACAGCATCAACGCCCCGGCCTATACTCGAACCCCACTTGCACTCTGCAATG
GTATGGGAACCACGGGGCAGTCTTGTGTGGGTCGCGCCTATCGCGGTCGGCGAA
GACCGGGAAggtaccgcggtgagaatcgaaaatgcatcgtttctaggttcggagacggtcaattccctgctccggcgaatctg
tcggtcaagctggccagtggacaatgttgctatggcagcccgcgcacatgggcctcccgacgcggccatcaggagcccaaacagc
gtgtcagggtatgtgaaactcaagaggtccctgctgggcactccggccccactccgggggcgggacgccaggcattcgcggtcggt
cccgcgcgacgagcgaaatgatgattcggttacgagaccaggacgtcgtcgaggtcgagaggcagcctcggacacgtctcgctag
ggcaacgccccgagtccccgcgagggccgtaaacattgtttctgggtgtcggagtgggcattttgggcccgatccaatcgcctcatgc
cgctctcgtctggtcctcacgttcgcgtacggcctggatcccggaaagggcggatgcacgtggtgttgccccgccattggcgcccac
gtttcaaagtccccggccagaaatgcacaggaccggcccggctcgcacaggccatgctgaacgcccagatttcgacagcaacacca
tctagaataatcgcaaccatccgcgttttgaacgaaacgaaacggcgctgtttagcatgtttccgacatcgtgggggccgaagcatgct
ccggggggaggaaagcgtggcacagcggtagcccattctgtgccacacgccgacgaggaccaatccccggcatcagccttcatcg
acggctgcgccgcacatataaagccggacgcctaaccggtttcgtggttatgactagtATGttcgcgttctacttcctgacggcctgc
atctccctgaagggcgtgacggcgtctccccctcctacaacggcctgggcctgacgccccagatgggctgggacaactggaaca
cgacgcctgcgacgtctccgagcagctgctgctggacacggccgaccgcatctccgacctgggcctgaaggacatgggctaca
agtacatcatcctggacgactgctggtcctccggccgcgactccgacggcacctggtcgccgacgagcagaagaccccaacgg
catgggccacgtcgccgaccacctgcacaacaactccacctgacggcatgtactcctccgcgggcgagtacacgtgcgccggct
accccggctccctgggccgcgaggaggaggacgcccagacttcgcgaacaaccgcgtggactacctgaagtacgacaactgc
tacaacaagggccagacggcacgcccgagatctcctaccaccgctacaaggccatgtccgacgccctgaacaagacgggccg
ccccatcttctactccctgtgcaactggggccaggacctgaccttctactggggctccggcatcgcgaactcctggcgcatgtccgg
cgacgtcacggcggagacacgcgccccgactcccgctgcccctgcgacggcgacgagtacgactgcaagtacgccggcacc
actgctccatcatgaacatcctgaacaaggccgcccccatgggccagaacgcgggcgtcggcggctggaacgacctggacaa
cctggaggtcggcgtcggcaacctgacggacgacgaggagaaggcgcacttaccatgtgggccatggtgaagtcccccctgat
catcggcgcgaacgtgaacaacctgaaggcctcctcctactccatctactcccaggcgtccgtcatcgccatcaaccaggactcc
aacggcatccccgccacgcgcgtctggcgctactacgtgtccgacacggacgagtacggccagggcgagatccagatgtggtc
cggccccctggacaacggcgaccaggtcgtggcgctgctgaacggcggctccgtgtcccgccccatgaacacgaccctggagg
agatcacttcgactccaacctgggctccaagaagctgacctccacctgggacatctacgacctgtgggcgaaccgcgtcgacaa
ctccacggcgtccgccatcctgggccgcaacaagaccgccaccggcatcctgtacaacgccaccgagcagtcctacaaggacg
gcctgtccaagaacgacacccgcctgacggccagaagatcggctccctgtcccccaacgcgatcctgaacacgaccgtccccg
cccacggcatcgcgactaccgcctgcgcccctcctccTGAtacgtactcgagGCAGCAGCAGCTCGGATAGT
ATCGACACACTCTGGACGCTGGTCGTGTGATGGACTGTTGCCGCCACACTTGCTG
CCTTGACCTGTGAATATCCCTGCCGCTTTTATCAAACAGCCTCAGTGTGTTTGATC
TTGTGTGTACGCGCTTTTGCGAGTTGCTAGCTGCTTGTGCTATTTGCGAATACCAC
CCCCAGCATCCCCTTCCCTCGTTTCATATCGCTTGCATCCCAACCGCAACTTATCT
ACGCTGTCCTGCTATCCCTCAGCGCTGCTCCTGCTCCTGCTCACTGCCCCTCGCAC
AGCCTTGGTTTGGGCTCCGCCTGTATTCTCCTGGTACTGCAACCTGTAAACCAGC
ACTGCAATGCTGATGCACGGGAAGTAGTGGGATGGGAACACAAATGGAAagctgtag
aattcctggctcgggcctcgtgctggcactccctcccatgccgacaacctttctgctgtcaccacgacccacgatgcaacgcgacacg
acccggtgggactgatcggttcactgcacctgcatgcaattgtcacaagcgcatactccaatcgtatccgtttgatttctgtgaaaactcg
ctcgaccgcccgcgtcccgcaggcagcgatgacgtgtgcgtgacctgggtgtttcgtcgaaaggccagcaaccccaaatcgcaggc
gatccggagattgggatctgatccgagcttggaccagatcccccacgatgcggcacgggaactgcatcgactcggcgcggaaccca
gctttcgtaaatgccagattggtgtccgataccttgatttgccatcagcgaaacaagacttcagcagcgagcgtatttggcgggcgtgct
accagggttgcatacattgcccatttctgtctggaccgctttaccggcgcagagggtgagttgatggggttggcaggcatcgaaacgc
gcgtgcatggtgtgtgtgtctgttttcggctgcacaatttcaatagtcggatgggcgacggtagaattgggtgttgcgctcgcgtgcatgc
ctcgccccgtcgggtgtcatgaccgggactggaatcccccctcgcgaccctcctgctaacgctcccgactctcccgcccgcgcgcag
gatagactctagttcaaccaatcgacaactagtATGgccatggccgccgccgtgatcgtgcccctgggcatcctgacttcatctcc
ggcctggtggtgaacctgctgcaggccatctgctacgtgctgatccgccccctgtccaagaacacctaccgcaagatcaaccgcg
tggtggccgagaccctgtggctggagctggtgtggatcgtggactggtgggccggcgtgaagatccaggtgacgccgacaacg
agaccacaaccgcatgggcaaggagcacgccctggtggtgtgcaaccaccgctccgacatcgactggctggtgggctggatcc
tggcccagcgctccggctgcctgggctccgccctggccgtgatgaagaagtcctccaagacctgcccgtgatcggctggtccatgt
ggactccgagtacctgacctggagcgcaactgggccaaggacgagtccaccctgaagtccggcctgcagcgcctgaacgactt
cccccgcccataggctggccctgacgtggagggcacccgatcaccgaggccaagctgaaggccgcccaggagtacgccgc
ctcctccgagctgcccgtgccccgcaacgtgctgatcccccgcaccaagggcttcgtgtccgccgtgtccaacatgcgctccttcgt
gcccgccatctacgacatgaccgtggccatccccaagacctcccccccccccaccatgctgcgcctgacaagggccagccctcc
gtggtgcacgtgcacatcaagtgccactccatgaaggacctgcccgagtccgacgacgccatcgcccagtggtgccgcgacca
gacgtggccaaggacgccctgctggacaagcacatcgccgccgacaccaccccggccagcaggagcagaacatcggccgc
cccatcaagtccctggccgtggtgctgtcctggtcctgcctgctgatcctgggcgccatgaagacctgcactggtccaacctgactc
ctcctggaagggcatcgccactccgccctgggcctgggcatcatcaccctgtgcatgcagatcctgatccgctcctcccagtccga
gcgctccacccccgccaaggtggtgcccgccaagcccaaggacaaccacaacgactccggctcctcctcccagaccgaggtg
gagaagcagaagTGAatcgatagatctcttaagGCAGCAGCAGCTCGGATAGTATCGACACACT
CTGGACGCTGGTCGTGTGATGGACTGTTGCCGCCACACTTGCTGCCTTGACCTGT
GAATATCCCTGCCGCTTTTATCAAACAGCCTCAGTGTGTTTGATCTTGTGTGTACG
CGCTTTTGCGAGTTGCTAGCTGCTTGTGCTATTTGCGAATACCACCCCCAGCATCC
CCTTCCCTCGTTTCATATCGCTTGCATCCCAACCGCAACTTATCTACGCTGTCCTG
CTATCCCTCAGCGCTGCTCCTGCTCCTGCTCACTGCCCCTCGCACAGCCTTGGTTT
GGGCTCCGCCTGTATTCTCCTGGTACTGCAACCTGTAAACCAGCACTGCAATGCT
GATGCACGGGAAGTAGTGGGATGGGAACACAAATGGAaagcttaattaagagctcAGCGG
CGACGGTCCTGCTACCGTACGACGTTGGGCACGCCCATGAAAGTTTGTATACCGA
GCTTGTTGAGCGAACTGCAAGCGCGGCTCAAGGATACTTGAACTCCTGGATTGAT
ATCGGTCCAATAATGGATGGAAAATCCGAACCTCGTGCAAGAACTGAGCAAACC
TCGTTACATGGATGCACAGTCGCCAGTCCAATGAACATTGAAGTGAGCGAACTGT
TCGCTTCGGTGGCAGTACTACTCAAAGAATGAGCTGCTGTTAAAAATGCACTCTC
GTTCTCTCAAGTGAGTGGCAGATGAGTGCTCACGCCTTGCACTTCGCTGCCCGTG
TCATGCCCTGCGCCCCAAAATTTGAAAAAAGGGATGAGATTATTGGGCAATGGA
CGACGTCGTCGCTCCGGGAGTCAGGACCGGCGGAAAATAAGAGGCAACACACTC
CGCTTCTTAgctcttc

Additional transforming constructs to test the activity of LPAATs from B. napus, T cacao, G. hombroriana and G. indica contained the same selectable marker, restriction sites, promoters and 3′ UTR elements as pSZ4198. The coding sequences of BnLPAT2(Bn1.5), TcLPAT2, GhomLPAT2A, GhomLPAT2B, GhomLPAT2C, GindLPAT2A, GindLPAT2B and GindLPAT2C are shown in below. In each case the initiator ATG and terminator TGA are indicated by uppercase italics; the sequence encoding the LPAT2 homolog is represented by lowercase italics. The Brassica napus LPAAT2(BN1.13) sequence is from Genbank accession GU045435. The Theobroma cacao LPAAT2 sequence is from the cocoaGenDB database.

SEQ ID NO: 89 Nucleotide sequence of the BnLPAT2(1.5) coding sequence,
used in the transforming DNA from pSZ4202
ATGgccatggccgccgccgccgtgatcgtgcccctgggcatcctgacttcatctccggcctggtggtgaacctgctgcaggccgt
gtgctacgtgctgatccgccccctgtccaagaacacctaccgcaagatcaaccgcgtggtggccgagaccctgtggctggagctg
gtgtggatcgtggactggtgggccggcgtgaagatccaggtgacgccgacgacgagaccacaaccgcatgggcaaggagca
cgccctggtggtgtgcaaccaccgctccgacatcgactggctggtgggctggatcctggcccagcgctccggctgcctgggctcc
gccctggccgtgatgaagaagtcctccaagacctgcccgtgatcggctggtccatgtggactccgagtacctgacctggagcgca
actgggccaaggacgagtccaccctgaagtccggcctgcagcgcctgaacgacacccccgccccactggctggccctgacgtg
gagggcacccgatcaccgaggccaagctgaaggccgcccaggagtacgccgcctcctcccagctgcccgtgccccgcaacgt
gctgatcccccgcaccaagggcttcgtgtccgccgtgtccaacatgcgctccttcgtgcccgccatctacgacatgaccgtggccat
ccccaagacctcccccccccccaccatgctgcgcctgacaagggccagccctccgtggtgcacgtgcacatcaagtgccactcc
atgaaggacctgcccgagtccgacgacgccatcgcccagtggtgccgcgaccagacgtggccaaggacgccctgctggacaa
gcacatcgccgccgacaccaccccggccagaaggagcacaacatcggccgccccatcaagtccctggccgtggtggtgtcctg
ggcctgcctgctgaccctgggcgccatgaagacctgcactggtccaacctgactcctccctgaagggcatcgccctgtccgccctg
ggcctgggcatcatcaccctgtgcatgcagatcctgatccgctcctcccagtccgagcgctccacccccgccaaggtggcccccg
ccaagcccaaggacaagcaccagtccggctcctcctcccagaccgaggtggaggagaagcagaagTGA
SEQ ID NO: 90 Nucleotide sequence of the TcLPAT2 coding sequence, used
in the transforming DNA from pSZ4206
ATGgccatcgccgccgccgccgtgatcgtgcccctgggcctgctgttcttcatctccggcctggtggtgaacctgatccaggccctgtgcttc
gtgctgatccgccccctgtccaagaacacctaccgcaagatcaaccgcgtggtggccgagctgctgtggctggagctgatctggctggtgg
actggtgggccggcgtgaagatcaaggtgttcatggaccccgagtccttcaacctgatgggcaaggagcacgccctggtggtggccaacc
accgctccgacatcgactggctggtgggctggctgctggcccagcgctccggctgcctgggctccgccctggccgtgatgaagaagtcctcc
aagttcctgcccgtgatcggctggtccatgtggttctccgagtacctgttcctggagcgctcctgggccaaggacgagaacaccctgaaggc
cggcctgcagcgcctgaaggacttcccccgccccttctggctggccttcttcgtggagggcacccgcttcacccaggccaagttcctggccgc
ccaggagtacgccgcctcccagggcctgcccatcccccgcaacgtgctgatcccccgcaccaagggcttcgtgtccgccgtgtcccacatgc
gctccttcgtgcccgccatctacgacatgaccgtggccatccccaagtcctccccctcccccaccatgctgcgcctgttcaagggccagccctc
cgtggtgcacgtgcacatcaagcgctgcctgatgaaggagctgcccgagaccgacgaggccgtggcccagtggtgcaaggacatgttcg
tggagaaggacaagctgctggacaagcacatcgccgaggacaccttctccgaccagcccatgcaggacctgggccgccccatcaagtcc
ctgctggtggtggcctcctgggcctgcctgatggcctacggcgccctgaagttcctgcagtgctcctccctgctgtcctcctggaagggcatcg
ccttcttcctggtgggcctggccatcgtgaccatcctgatgcacatcctgatcctgttctcccagtccgagcgctccacccccgccaaggtggc
ccccggcaagcccaagaacgacggcgagacctccgaggcccgccgcgacaagcagcagTGA
SEQ ID NO: 91 Nucleotide sequence of the GhomLPAT2A coding sequence,
used in the transforming DNA from pSZ4412.
ATGgccatccccgccgccatcgtgatcgtgcccgtgggcctgctgttcttcatctccggcctgatcgtgaacctgctgcaggccctgtgcttcg
tgctgatccgccccctgtccaagtccgcctaccgcaccatcaaccgccagctggtggagctgctgtggctggagctggtgtgcatcgtggac
tggtgggcccgcgtgaagatccagctgttcaccgacaaggagaccctgaactccatgggcaaggagcacgccctggtgatgtgcaacca
ccgctccgacatcgactggctggtgggctggatcctggcccagcgctccggctgcctgggctccaccgtggccgtgatgaagaagtcctcca
aggtgctgcccgtgatcggctggtccatgtggttctccgagtacctgttcctggagcgcaactgggccaaggacgagtccaccctgaagtcc
ggcctgcagcgcctgcgcgacttcccccgccccttctggctggccctgttcgtggagggcacccgcttcacccagcccaagctgctggccgcc
caggagtacgccgcctccaccggcctgcccatcccccgcaacgtgctgatcccccgcaccaagggcttcgtgtccgccgtgtccatcacccgc
tccttcgtgcccgtgatctacgacatcaccgtggccatccccaagtcctccccccagcccaccatgctgcgcctgttcaagggccagtcctccg
tggtgcacgtgcacctgaagcgccacctgatgaaggacctgcccgagtccgacgacgacgtggcccagtggtgccgcgaccagttcgtgg
tgaaggactccctgctggacaagcacatcgccgaggacaccttctccgaccaggagctgcaggacatcggccgccccatcaagtccctgg
tggtgttcacctcctgggtgtgcatcatcaccttcggcgccctgaagttcctgcagtggtcctccctgctgcactcctggaagggcatcgccat
ctccgcctccggcctggccatcgtgaccgtgctgatgcacatcctgatccgcttctcccagtccgagcactccacctccgccaagatcgccgcc
gagaagcacaagaacggcggcgtgtcccaggagatgggccgcgagaagcagcacTGA
SEQ ID NO: 92 Nucleotide sequence of the GhomLPAT2B coding sequence,
used in the transforming DNA from pSZ4413.
ATGgagatccccgccgtggccgtgatcgtgcccatcggcatcctgttcttcatctccggcctgatcgtgaacctgatgcaggccatctgcttc
ttcctgatccgccccctgtccaagaacacccaccgcatcgtgaaccgccagctggccgagctgctgtggctggagctgatctggatcgtgga
ctggtgggccggcgtgaagatccagctgttcaccgacaaggagaccctgcacctgatgggcaaggagcacgccctggtgatctgcaacc
actcctccgacatcgactggctggtgggctggctgctgtgccagcgctccggctgcctgggctccgccctggccgtgatgaagtcctcctcca
aggtgctgcccgtgatcggctggtccatgtggttctccgagtacctgttcctggagcgctcctgggccaaggacgagtccaccctgaagtcc
ggcctgcagcgcctgaaggacttcccccgccccttctggctggccctgttcgtggagggcacccgcttcacccaggccaagctgctggccgc
ccaggagtacgccatgtccgccggcctgcccgtgccccgcaacgtgctgatcccccgcaccaagggcttcgtgtccgccgtgtccaacatgc
gctccttcgtgcccgccatctacgacgtgaccgtggccatccccaagtcctccgtgcagcccaccatgctgcgcctgttcaagggccagtcctc
cgtggtgcaggtgcacctgaagcgccactccatgaaggacctgcccgagtccgaggacgacgtggcccagtggtgccgcgaccgcttcgt
ggtgaaggactccctgctggacaagcacaaggtggaggacaccttcaccgaccaggagctgcaggacctgggccgccccatcaagtccc
tggtggtggtgacctgctgggcctgcatcatcatcttcggcatcctgaagttcctgcagtggtcctccctgctgtactcctggaagggcatggc
catctccgcctccggcctggccgtggtgaccttcctgatgcagatcctgatccgcttctcccagtccgagcgctccacccccgccaagatcgcc
cccgccaagcccaacaaggccggcaactcctccgagaccgtgcgcgacaagcaccagTGA
SEQ ID NO: 93 Nucleotide sequence of the GhomLPAT2C coding sequence,
used in the transforming DNA from pSZ4414.
ATGgccatccccgccgccatcatcatcgtgcccctgggcctgatcttcttcacctccggcctgatcatcaacctgatccaggccgtgtgctacg
tgctgatccgccccctgtccaagtccaccttccgccgcatcaaccgcgagctggccgagctgctgtggctggagctggtgtgggtggtggac
tggtgggccggcgtgaagatccagctgttcaccgacaaggagaccctgcactccatgggcaaggagcacgccctggtgatctgcaaccac
cgctccgacatcgactggctggtgggctggatcctggcccagcgctccggctgcctgggctccgccctggccgtgatgaagaagtcctccaa
ggtgctgcccgtgatcggctggtccatgtggttctccgagtacttcttcctggagcgcaactgggccatggacgagtccaccctgaagtccg
gcctgcagcgcctgaaggacttcccccagcccttctggctggccctgttcgtggagggcacccgcttcacccagcccaagctgctggccgccc
aggagtacgccgcctccgccggcctgcccatcccccgcaacgtgctgatcccccgcaccaagggcttcgtgtccgccgtgaacatcatgcgc
tccttcgtgcccgccatctacgacgtgaccgtggccatccccaagtcctccccccagcccaccatgctgcgcctgttcaagggccagtcctccg
tggtgcacgtgcacctgaagcgccacctgatggaggacctgcccgagaccgacgacgacgtggcccagtggtgccgcgaccgcttcgtgg
tgaaggactccctgctggacaagtacgtggccgaggacaccttctccgaccaggagctgcaggacctgggccgccccatcaagtccctgg
tggtggtgacctcctgggtgtgcatcatcgccttcggctccctgaagttcctgcagtggtcctccctgctgtactcctggaagggcatcgtgat
ctccgccgcctccctggccgtggtgaccgtgctgatgcagatcctgatccgcttctcccagtccgagcgctccacctccgccaagatcgccgcc
gccaagcgcaagaacgtgggcgagcacTGA
SEQ ID NO: 94 Nucleotide sequence of the GindPAT2A coding sequence,
used in the transforming DNA from pSZ4415.
ATGgccatccccgtggtggtggtgatcgtgcccgtgggcctgctgttcttcatctccggcctgatcgtgaacctgctgcaggccctgtgcttc
gtgctgatccgccccctgtccaagtccgcctaccgcaccatcaaccgccagctggtggagctgctgtggctggagctggtgtgcatcgtgga
ctggtgggcccgcgtgaagatccagctgttcatcgacaaggagaccctgaactccatgggcaaggagcacgccctggtgatgtgcaacc
accgctcctacatcgactggctggtgggctggatcctggcccagcgctccggctgcctgggctccaccgtggccgtgatgaagaagtcctcc
aaggtgctgcccgtgatcggctggtccatgtggttctccgagtacctgttcctggagcgcaactgggccaaggacgagtccaccctgaagt
ccggcctgcagcgcctgcgcgacttcccccgccccttctggctggccctgttcgtggagggcacccgcttcacccagcccaagctgctggccg
cccaggagtacgccgcctccaccggcctgcccatcccccgcaacgtgctgatcccccgcaccaagggcttcgtgtccgccgtgtccatcaccc
gctccttcgtgcccgtgatctacgacatcaccgtggccatccccaagtcctcctcccagcccaccatgctgaagctgttcaagggccagtcctc
cgtggtgcacgtgcacctgaagcgccacctgatgaaggacctgcccgagtccgacgacgacgtggcccagtggtgccgcgcccagttcgt
ggtgaaggactccctgctggacaagcacatcgccgaggacaccttctccgaccaggagctgcaggacatcggccgccccatcaagtccct
ggtggtgttcacctcctgggtgtgcatcatcaccttcggcgccctgaagttcctgcagtggtcctccctgctgcactcctggaagggcatcgcc
atctccgcctccggcctggccatcgtgaccgtgctgatgcacatcctgatccgcttctcccagtccgagcactccacctccgccaagatcgccg
ccgagaagcacaagaacggcggcgtgtcccaggagatgggccgcgagaagcagcacTGA
SEQ ID NO: 95 Nucleotide sequence of the GindPAT2B coding sequence,
used in the transforming DNA from pSZ4416.
ATGggcatccccgccgtggccgtgatcgtgcccatcggcatcctgttcttcatctccggcttcatcgtgaacctgatgcaggccatctgcttcg
tgctgatccgccccctgtccaagaacacctaccgcatcgtgaaccgccagctggccgagttcctgtggctggagctgatctgggtggtggac
tggtgggccggcgtgaagatccagctgttcaccgacaaggagaccctgcacctgatgggcaaggagcacgccctggtgatctgcaacca
ccgctccgacatcgactggctggtgggctggctgctgtgccagcgctccggctgcctgggctccgccctggccgtgatgaagtcctcctccaa
ggtgctgcccgtgatcggctggtccatgtggttctccgagtacctgttcctggagcgctcctgggccaaggacgagtccaccctgaagctgg
gcctgcagcgcctgaaggacttcccccgccccttctggctggccctgttcgtggagggcacccgcttcacccaggccaagctgctggccgccc
aggagtacgccatgtccgccggcctgcccgtgccccgcaacgtgctgatcccccgcaccaagggcttcgtgtccgccgtgtccaacatgcgc
tccttcgtgcccgccatctacgacgtgaccgtggccatccccaagtcctccgtgcagcccaccatgctgggcctgttcaagggccagtcctgc
gtggtgcaggtgcacctgaagcgccacctgatgaaggacctgcccgagtccgaggacgacgtggcccagtggtgccgcgagcgcttcgt
ggtgaaggactccctgctggacaagcacaaggtggaggacaccttctccgaccaggagctgcaggacctgggccgccccatcaagtccct
ggtggtggtgatctcctgggcctgcatcctgatcttctggatcctgaagttcctgcagtggtcctccctgctgtactcctggaagggcatcgcc
atctccgcctgcgccatggccgtgatcgccttcctgatgcagatcctgctgcgcttctcccagtccgagcgctccacccccgccaagatcgccc
ccgccaagcccaacaacgcccgcaactcctccgagaccgtgcgcgacaagcaccagTGA
SEQ ID NO: 96 Nucleotide sequence of the GindPAT2C coding sequence,
used in the transforming DNA from pSZ4417.
ATGgccatccccgccgccatcatcatcgtgcccctgggcctgatcttcttcacctccggcttcatcatcaacctgatccaggccgtgtgctacg
tgctgatccgccccctgtccaagtccaccttccgccgcatcaaccgccagctggccgagctgctgtggctggagctggtgtgggtggtggac
tggtgggccggcgtgaagatccagctgttcaccaacaaggagaccctgcactccatcggcaaggagcacgccctggtgatctgcaaccag
cgctccgacatcgactggctggtgggctggatcctggcccagcgctccggctgcctgggctccgccctggccgtgatgaagaagtcctccaa
ggtgctgcccgtgatcggctggtccatgtggttctccgagtacctgttcctggagcgcaactgggccatggacgagtccaccctgaagtccg
gcctgcagtggctgaaggacttcccccagcccttctggctggccctgttcgtggagggcacccgcttcacccagcccaagctgctggccgcc
caggagtacgccgcctccgccggcctgcccatcccccgcaacgtgctgatcccccgcaccaagggcttcgtgtccgccgtgaacatcatgcg
ctccttcgtgcccgccgtgtacgacgtgaccgtggccatccccaagtcctccccccagcccaccatgctgcgcctgttcaagggccagtcctcc
gtggtgcacgtgcacctgaagcgccacctgatggaggacctgcccgagaccgacgacgacgtggcccagtggtgccgcgaccgcttcgtg
gtgaaggactccctgctggacaagcacctggccgaggacaccttctccgaccaggagctgcaggacctgggccgccccatcaagtccctg
gtggtggtgacctcctgggtgtgcatcatcgccttcggcgccctgaagttcctgcagtggtcctccctgctgtactcctggaagggcatcgtg
atctccgccgcctccctggccgtggtgaccgtgctgatgcagatcctgatccgcttctcccagtccgagcgctccacctccgccaaggtggtg
gccgagaagcgcaagaacgtgggcgagcacTGA

Constructs D2971, D2973, D2975, D3219, D3221, D3223, D3225, D3227 and D3229, derived from pSZ4198, pSZ4202, pSZ4206, pSZ4412, pSZ4413, pSZ4414, pSZ4415, pSZ4416 and pSZ4417, respectively, were transformed into the S6573 parent strain. The fatty acid profiles of primary transformants are shown in Table 10. Also shown are the SOS/SSS ratios determined by LC/MS multiple response measurements. Expression of LPAT2 genes had no discernable effect on C16:0 or C18:0 accumulation, but C18:2 levels increased by 1-2% compared to the S6573 parent in strains when expressing the D2971, D2973, D2975, D3221, D3223, and D3227 constructs. Expression of LPAT2 genes increased C18:2 and also elevated ratios of SOS/SSS, showing reduced accumulation of trisaturated TAGs.

TABLE 10
Fatty acid profiles and SOS/SSS ratios of D2971, D2973, D2975,
D3219, D3221, D3223, D3225, D3227 and D3229 primary transformants.

| Strain | LPAAT gene | SOS/SSS | C14:0 | C16:0 | C18:0 | C18:1 | C18:2 | C18:3 α | C20:0 | saturates |
|---|---|---|---|---|---|---|---|---|---|---|
| S5100 | | | 0.7 | 17.7 | 4.1 | 68.5 | 6.8 | 0.6 | 0.4 | 23.3 |
| S6573.1 | | 15 | 0.8 | 6.2 | 50.7 | 33.7 | 5.6 | 0.7 | 1.5 | 59.8 |
| D2971.1 | BnLPAT2(1.13) | 23 | 0.8 | 6.1 | 51.4 | 30.5 | 8.6 | 0.6 | 1.4 | 60.2 |
| D2971.2 | | 16 | 0.8 | 6.1 | 54.3 | 28.9 | 7.0 | 0.6 | 1.5 | 63.3 |
| D2971.4 | | 16 | 0.8 | 6.4 | 53.3 | 29.5 | 7.3 | 0.6 | 1.4 | 62.6 |
| S6573.2 | | 14 | 0.8 | 6.6 | 52.8 | 31.7 | 5.2 | 0.6 | 1.5 | 62.3 |
| D2973.2 | BnLPAT2(1.5) | 22 | 0.8 | 6.2 | 53.4 | 28.3 | 6.4 | 0.6 | 1.7 | 62.7 |
| D2973.38 | | 23 | 0.9 | 7.5 | 51.2 | 29.1 | 6.5 | 0.5 | 1.4 | 61.7 |
| D2973.24 | | 24 | 0.9 | 6.8 | 51.7 | 29.2 | 6.3 | 0.5 | 1.6 | 61.5 |
| S6573.3 | | 14 | 0.8 | 6.6 | 52.8 | 31.7 | 5.2 | 0.6 | 1.5 | 62.3 |
| D2975.33 | TcLPAT2 | 27 | 0.8 | 6.6 | 52.7 | 29.7 | 7.1 | 0.6 | 1.5 | 62.3 |
| D2975.13 | | 32 | 0.8 | 6.5 | 52.4 | 30.2 | 7.3 | 0.6 | 1.4 | 61.7 |
| D2975.35 | | 27 | 0.8 | 6.5 | 52.8 | 29.6 | 7.3 | 0.6 | 1.5 | 62.2 |
| S6573.4 | | 12 | 0.9 | 6.4 | 54.9 | 28.9 | 5.7 | 0.6 | 1.7 | 64.5 |
| D3219.19 | GhomLPAT2A | 12 | 0.9 | 7.1 | 52.4 | 31.2 | 4.8 | 0.5 | 2.0 | 63.1 |
| D3219.20 | | 14 | 0.9 | 6.6 | 53.2 | 30.6 | 5.5 | 0.6 | 1.7 | 63.0 |
| D3219.32 | | 15 | 0.8 | 6.4 | 53.1 | 29.8 | 6.5 | 0.6 | 1.5 | 62.6 |
| S6573.5 | | 12 | 0.9 | 6.4 | 53.7 | 30.3 | 5.5 | 0.6 | 1.6 | 63.3 |
| D3220.1 | GhomLPAT2B | 27 | 0.9 | 6.6 | 52.2 | 30.0 | 7.0 | 0.7 | 1.4 | 61.9 |
| D3221.39 | | 20 | 0.9 | 6.7 | 53.9 | 28.7 | 6.7 | 0.6 | 1.5 | 63.7 |
| D3221.40 | | 22 | 0.8 | 6.5 | 53.7 | 29.1 | 6.8 | 0.6 | 1.4 | 63.2 |
| S6573.6 | | 14 | 0.8 | 6.3 | 54.0 | 30.2 | 5.5 | 0.6 | 1.6 | 63.4 |
| D3223.2 | GhomLPAT2C | 20 | 0.8 | 6.5 | 53.0 | 29.3 | 7.3 | 0.6 | 1.5 | 62.4 |
| D3223.6 | | 21 | 0.8 | 6.5 | 53.5 | 29.3 | 7.0 | 0.6 | 1.4 | 62.7 |
| D3223.7 | | 21 | 0.8 | 6.4 | 52.5 | 30.7 | 6.6 | 0.5 | 1.5 | 61.8 |
| D3225.5 | GindLPAT2A | 13 | 0.9 | 6.6 | 53.5 | 30.2 | 5.6 | 0.6 | 1.6 | 63.2 |
| S6573.7 | | 12 | 0.9 | 6.5 | 53.5 | 29.9 | 5.7 | 0.6 | 1.8 | 63.3 |
| D3227.6 | GindLPAT2B | 23 | 0.8 | 6.4 | 54.1 | 28.8 | 6.8 | 0.6 | 1.6 | 63.5 |
| D3227.3 | | 21 | 0.8 | 6.5 | 53.9 | 29.0 | 6.7 | 0.6 | 1.5 | 63.4 |
| D3227.17 | | 22 | 0.8 | 6.6 | 53.8 | 28.8 | 7.0 | 0.6 | 1.4 | 63.3 |
| S6573.8 | | 11 | 0.8 | 6.4 | 54.3 | 30.1 | 5.4 | 0.6 | 1.7 | 63.8 |
| D3229.41 | GindLPAT2C | 11 | 0.9 | 6.6 | 54.2 | 29.7 | 5.6 | 0.6 | 1.7 | 63.9 |
| D3229.27 | | 13 | 0.8 | 6.4 | 54.1 | 30.0 | 5.6 | 0.6 | 1.7 | 63.6 |
| D3229.33 | | 12 | 0.8 | 6.4 | 54.0 | 30.2 | 5.5 | 0.6 | 1.7 | 63.5 |

Table 11 presents the TAG composition of the lipids produced by D2971, D2973, D2975, D3221, D3223, and D3227 primary transformants relative to the S6573 parent. SOS levels in the LPAT2-expressing strains were equivalent or slightly higher than in the S6573 controls. Trisaturates declined by up to 53%, and total Sat-Unsat-Sat levels improved in all of the strains expressing heterologous LPAT2 genes. Among the LPAT2 genes, the strains expressing the T. cacao LPAT2 homolog showed the greatest improvements in their TAG profiles).

TABLE 11
TAG composition of D2971, D2973, D2975, D3221, D3223,
and D3227 primary transformants relative to the S6573 parent.

| | | LPAAT gene | | | | | | | | |
|---|---|---|---|---|---|---|---|---|---|---|
| | | BnLPAT2 | BnLPAT2 | | | Ghom | | Ghom | Gind | |
| | | (1.13) | (1.5) | TcLPAT2 | | LPAT2B | | LPAT2C | LPAT2B | |
| | | Strain | | | | | | | | |
| | | D2971.1 | D2973.38 | D2975.33 | D2975.13 | D3221.39 | D3221.40 | D3223.6 | D3227.3 | D3227.6 |
| % S6573 | SOS | 100 | 100 | 110 | 104 | 107 | 107 | 108 | 103 | 105 |
| TAG | Sat-Sat-Sat | 57 | 63 | 48 | 47 | 74 | 62 | 68 | 62 | 70 |
| | Sat-U-Sat | 109 | 107 | 113 | 110 | 112 | 112 | 109 | 108 | 107 |
| | Sat-O-Sat | 97 | 100 | 105 | 102 | 106 | 105 | 102 | 104 | 104 |
| | Sat-L-Sat | 174 | 147 | 155 | 155 | 139 | 143 | 141 | 130 | 125 |
| | U-U-U/Sat | 85 | 86 | 72 | 83 | 64 | 69 | 78 | 82 | 79 |

We analyzed the fatty acid profiles, TAG profiles and lipid titers from 50 mL shake flask cultures of stable lines generated from D2975-33. C18:0 and C16:0 levels were comparable between the strains and the S6573 control, and lipid titers ranged from 75-105% of the parent strain titer (Table 12). C18:2 levels increased by more than 2% in the TcLPAT2-expressing strains.

TABLE 12
Fatty acid profiles of TcLPAT2-expressing stable lines made
from D2975-33.

| Primary | | D1940.19 | D2975.33 | | | | |
|---|---|---|---|---|---|---|---|
| Strain | | S6573 | S7813 | S7815 | S7816 | S7817 | S7819 |
| Fatty Acid | C12:0 | 0.2 | 0.2 | 0.2 | 0.2 | 0.2 | 0.2 |
| Area % | C14:0 | 0.9 | 0.7 | 0.8 | 0.8 | 0.7 | 0.7 |
| | C16:0 | 6.5 | 5.9 | 6.1 | 5.9 | 6.1 | 6.0 |
| | C16:1 | 0.1 | 0.1 | 0.1 | 0.1 | 0.1 | 0.1 |
| | cis-9 | | | | | | |
| | C17:0 | 0.2 | 0.2 | 0.2 | 0.2 | 0.2 | 0.2 |
| | C18:0 | 56.1 | 55.6 | 55.9 | 56.2 | 53.9 | 53.9 |
| | C18:1 | 28.1 | 26.8 | 26.6 | 26.5 | 28.8 | 28.4 |
| | C18:2 | 5.5 | 8.1 | 7.7 | 7.9 | 7.7 | 7.8 |
| | C18:3 α | 0.6 | 0.5 | 0.6 | 0.5 | 0.6 | 0.7 |
| | C20:0 | 1.5 | 1.5 | 1.4 | 1.3 | 1.3 | 1.5 |
| | C22:0 | 0.2 | 0.2 | 0.1 | 0.1 | 0.1 | 0.2 |
| | C24:0 | 0.1 | 0.1 | 0.1 | 0.1 | 0.1 | 0.1 |
| | saturates | 65.7 | 64.4 | 65.0 | 64.9 | 62.8 | 62.9 |

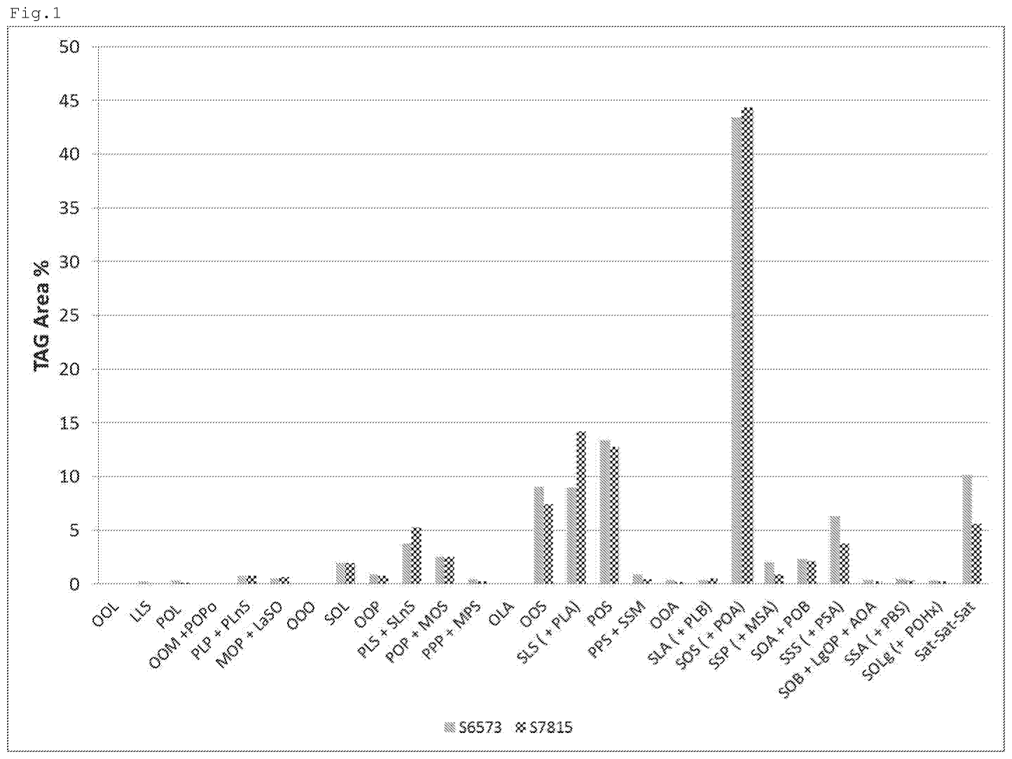

The TAG profiles of S6573 and S7815 are compared in FIG. 1. SOS levels in the LPAT2-expressing strains were higher than in the S6573 control. Trisaturates were reduced from 10.2% in S6573 to 5.6% in S7815. Much of the improvement in total sat-unsat-sat levels in S7815 came from a 4% increase in stearate-linoleate-stearate (SLS) and a 1.5% increase in palmitate-linoleate-stearate (PLS), consistent with the enhanced C18:2 content of that strain. These results indicate that the T. cacoa LPAT2 reduces the incorporation of saturated fatty acids at the sn-2 position.

The performance of S7815 versus the S6573 parent strain was compared in high-density fermentations. The fatty acid profile of each strain at the two time points of the fermentations are shown in Table 13. The strains had very similar composition, with 5.5-5.7% C16:0, 56.4-56.8% C18:0, and 27.2-28.6% C18:1 as the major fatty acids. As was observed in the shake flask assays, (see Table 12), C18:2 levels increased from 5.5% in S6573 to 7.7% in S7815(Table 13). Normalized lipid titers and yields were comparable between the two strains, indicating that expression of the TcLPAT2 gene in S7815 did not have deleterious effects on growth or lipid accumulation.

TABLE 13
Fatty acid profiles of S7815 versus S6573 fermentations.

| Strain | | S6573 | | S7815 | |
|---|---|---|---|---|---|
| Fermentation | | 140207F25 | | 140208F26 | |
| Fatty Acid | C12:0 | 0.19 | 0.20 | 0.20 | 0.21 |
| Area % | C14:0 | 0.71 | 0.72 | 0.66 | 0.66 |
| | C16:0 | 5.69 | 5.73 | 5.57 | 5.54 |
| | C16:1 cis-7 | 0.05 | 0.05 | 0.05 | 0.06 |
| | C16:1 cis-9 | 0.07 | 0.06 | 0.05 | 0.05 |
| | C17:0 | 0.11 | 0.11 | 0.12 | 0.11 |
| | C8:0 | 56.01 | 56.78 | 55.50 | 56.37 |
| | C8:1 | 29.31 | 28.58 | 27.92 | 27.19 |
| | C8:2 | 5.56 | 5.51 | 7.75 | 7.70 |
| | C8:3 α | 0.34 | 0.32 | 0.40 | 0.37 |
| | C20:0 | 1.51 | 1.50 | 1.35 | 1.34 |
| | C22:0 | 0.16 | 0.16 | 0.14 | 0.14 |
| | C24:0 | 0.10 | 0.09 | 0.09 | 0.08 |
| | sum C18 | 91.22 | 91.19 | 91.57 | 91.63 |
| | saturates | 64.54 | 65.34 | 63.69 | 64.51 |
| | unsaturates | 35.46 | 34.64 | 36.30 | 35.49 |

Table 13 compares the TAG profiles of the lipids produced during high-density fermentation of S7815 versus S6573. SOS and Sat-Oleate-Sat levels were almost identical between S7815 and the S6573 control. However, Sat-Linoleate-Sat levels increased by more than 7%, and di-unsaturated and tri-unsaturated TAGs (U—U-U/Sat) declined by more than 3% in S7815 compared to S6573. Trisaturates at the end points of the fermentations were reduced from 10.1% in S6573 to 6.1% in S7815. These results indicate that the activity of T. cacoa LPAT2 drives the transfer of unsaturated fatty acids towards the sn-2 position and discriminates against the incorporation of saturated fatty acids at sn-2.

### Example 6: Identification and Expression of Novel LPAAT, GPAT, DGAT, LPCAT and PLA2 with Specificity for Mid-Chain Fatty Acids

In this example, we demonstrate the effect of expression of LPAAT, GPAT, DGAT, LPCAT and PLA2 enzymes involved in triacylglycerol biosynthesis (in previously described P. moriformis (UTEX 1435) transgenic strains, S7858 and S8174. S7858 and S8174 were prepared according to co-owned WO2015/051319, herein incorporated by reference. In addition co-owned WO2010/063031 and WO2010/063032 teach the expression Cuphea hookerianas FATB2. Briefly, strain S7858 is a strain that express sucrose invertase and a Cuphea. hookeriana FATB2. To make S7858, the construct pSZ4329 (SEQ ID NO: 197) was engineered into S3150, a strain classically mutagenized to increase lipid yield. The plasmid, pSZ4329 is written as THI4α::CrTUB2-ScSUC2-PmPGH:PmAcp-P1p-CpSAD1tp_trimmed_ChFATB2_FLAG-CvNR::THI4a The annotation of the coding portions of pSZ4329 is shown in the Table A below.

TABLE A

| | | Nucleotide | Nucleotide | Nucleotide |
|---|---|---|---|---|
| pSZ4329 | Identity | Number | Number | Length |
| THI4a 3′ flank | 3′ flanking sequences | 5,692 | 6,394 | 703 |
| | of endogenous THI4 | | | |
| CvNR | 3′UTR | 5,278 | 5,679 | 402 |
| ChFATB2 | CDS | 4,105 | 5,271 | 1,167 |
| CpSAD1tp-trimmed | CDS | 3,991 | 4,104 | 114 |
| PmACP-P1 promoter | promoter | 3,411 | 3,981 | 571 |
| Buffer DNA | | 3,199 | 3,404 | 206 |
| UTR04424=PmPGH | 3′UTR | 2,749 | 3,192 | 444 |
| UTR | | | | |
| ScSUC2(o) | CDS | 1,144 | 2,742 | 1,599 |
| CrTUB2 promoter | promoter | | | |
| THI4a 5′ flank | 5′ flanking sequences | 820 | 1,131 | 312 |
| | of endogenous THI4 | 27 | 813 | 787 |

Strain S7858, accumulates C8:0 fatty acids to about 12% and C10:0 fatty acids to about 22-24%. Briefly, strain S8174 is a strain that express sucrose invertase and a Cuphea. Avigera var. pulcherrima FATB2. To make S8174, the construct pSZ5078 (SEQ ID NO: 198) was engineered into S3150, a strain classically mutagenized to increase lipid yield. pSZ5078 is written as THI4a5′::CrTUB2_ScSUC2_PmPGH:PmAMT3_CpSAD1tp_trimmed-CaFATB1_Flag_CvNR::THI4a3′. Strain S8174 accumulates C8:0 fatty acids to about 24% and C10:0 fatty acids to about 10%. The annotation of the coding portions of pSZ5078 is shown in the Table B below.

TABLE B

| | | Nucleotide | Nucleotide | Nucleotide |
|---|---|---|---|---|
| pSZ5078 | Identity | Number | Number | Length |
| THI4a 3′ | 3′ flanking sequences | 6,200 | 6,902 | 703 |
| flank | of endogenous THI4 | | | |
| CvNR | 3′UTR | 5,786 | 6,187 | 402 |
| CaFATB1 | CDS | 4,602 | 5,771 | 1,170 |
| wild-type | | | | |
| CpSAD1tp | CDS | 4,488 | 4,601 | 114 |
| AMT3 | promoter eukaryotic | 3,411 | 4,481 | 1,071 |
| Buffer DNA | misc_feature | 3,199 | 3,404 | 206 |
| PmPGH | 3′UTR | 2,749 | 3,192 | 444 |
| ScSUC2(o) | CDS | 1,144 | 2,742 | 1,599 |
| CrTUB2 | promoter | 820 | 1,131 | 312 |
| promoter | | | | |
| THI4a 5′ | 5′ flanking sequences | 27 | 813 | 787 |
| flank | of endogenous THI4 | | | |

The pool of acyl-CoAs in the ER can be utilized for the synthesis of TAGs as well as phospholipids and long chain fatty acids. The enzymes involved in the synthesis of TAGS and phospholids actively compete against each other for the same substrates. Acyl-CoAs can associate with lysophosphatidate to form phosphatidate which is converted to phosphatidylcholine (PC) and other phospholipid species. PC can be desaturated by FAD2 and FAD3 enzymes to generate polyunsaturated fatty acids, which can be cleaved by phosphotransferases and reenter the acyl-CoA pool. Acyl-CoAs can also be generated from PC directly by acyl-CoA:lysophosphatidylcholine acyltransferase (LPCAT). LPCAT can also catalyze the reverse reaction to consume acyl-CoA. Removal of fatty acids from PC to form acyl-CoAs can also be catalyzed by phospholipase A2 (PLA2). TAG formation in the ER from acyl-CoAs requires action of glycerol phosphate acyltransferase (GPAT), lysophosphatidic acid acyltransferase (LPAAT) and diacyl glycerol acyltransferase (DGAT).

The endogenous P. moriformis TAG biosynthesis machinery has evolved to function with the longer chain fatty acids that the strain normally makes. We introduced heterologous acyltransferases and phospholipases from species that naturally accumulate high levels of short chain fatty acids into Prototheca to increase accumulation of C8:0 fatty acids. We identified the following plant enzymes in NCBI as shown in Table 14 below.

TABLE 14
Genes representing target enzymes identified from higher plants
that produce high amounts of C8:0 and C10:0. All these genes were
synthesized with codon usage optimized for expression in Prototheca.

| Species | Gene | Enzyme |
|---|---|---|
| [symbol] | cnLPAAT1 | LPAAT |
| Cuphea [symbol] | [symbol] LPAAT1 | |
| Cuphea [symbol] | [symbol] LPAAT1 | |
| Cuphea [symbol] | [symbol] LPAAT1 | |
| Cuphea [symbol] | [symbol] LPAAT1 | |
| Cuphea [symbol] | [symbol] LPAAT1 | |
| Cuphea avigera var. [symbol] | [symbol] LPAAT1 | |
| Cuphea avigera var. [symbol] | [symbol] LPAAT2 | |
| Cuphea [symbol] | [symbol] LPAAT1 | |
| Cuphea [symbol] | [symbol] LPAAT1 | |
| Cuphea [symbol] | [symbol] LPAAT2 | |
| Cuphea [symbol] | [symbol] LPAAT2 | |
| Cuphea [symbol] | [symbol] LPAAT2 | |
| Cuphea avigera var. [symbol] | [symbol] GPAT9 [symbol] | GPAT |
| Cuphea [symbol] | [symbol] GPAT9 [symbol] 1 | |
| Cuphea [symbol] | [symbol] GPAT9 [symbol] 2 | |
| Cuphea [symbol] | [symbol] GPAT9 [symbol] 2 | |
| Cuphea [symbol] | [symbol] GPAT9 [symbol] 2 | |
| Cuphea [symbol] | [symbol] GPAT9 [symbol] 2 | |
| Cuphea avigera var. [symbol] | [symbol] DGAT1 | DGAT |
| Cuphea [symbol] | [symbol] DGAT1 [symbol] 1 | |
| Cuphea avigera var. [symbol] | [symbol] LPCAT | LPCAT |
| Cuphea [symbol] | [symbol] LPCAT | |
| Cuphea [symbol] | [symbol] LPCAT | |
| Cuphea [symbol] | [symbol] LPCAT1 | |
| Cuphea avigera var. [symbol] | [symbol] PLA2 [symbol] 1 | PLA2 |
| Cuphea [symbol] | [symbol] PLA2 [symbol] 1 | |
| Cuphea [symbol] | [symbol] PLA2 [symbol] 2 | |
| Cuphea [symbol] | [symbol] PLA2 [symbol] 2 | |

[symbol]

indicates data missing or illegible when filed

We made a set of constructs expressing heterologous short chain specific acyltransferases and PLA2s as shown in Table 15. The genes were codon optimized to reflect UTEX 1435 codon usage.

TABLE 15
List of constructs transformed into S7858 or S8174

| D# | Strain | Construct |
|---|---|---|
| D4289 | S7858 | SAD2-1vD::CpauLPAAT1 [symbol] PmATP:PmHXT1 [symbol] ScarMEL-PmPGK::SAD2Bex |
| D4290 | S7858 | SAD2-1vD:: [symbol] LPAAT1 [symbol] PmATP:PmHXT1 [symbol] ScarMEL-PmPGK::SAD2Bex |
| D4291 | S7858 | SAD2-1vD::CigneaLPAAT1 [symbol] PmATP:PmHXT1 [symbol] ScarMEL-PmPGK::SAD2Bex |
| D4292 | S7858 | SAD2-1vD:: [symbol] LPAAT1 [symbol] PmATP:PmHXT1 [symbol] ScarMEL-PmPGK::SAD2Bex |
| D4293 | S7858 | SAD2-1vD::ChookLPAAT1 [symbol] PmATP:PmHXT1 [symbol] ScarMEL-PmPGK::SAD2Bex |
| D4404 | S7858 | SAD2-1vD::CnLPAAT1 [symbol] PmATP:PmHXT1 [symbol] ScarMEL-PmPGK::SAD2Bex |
| D4517 | S8174 | SAD2-1vD::CavigLPAAT1 [symbol] PmATP:PmHXT1 [symbol] ScarMEL-PmPGK::SAD2Bex |
| D4518 | S8174 | SAD2-1vD::CavigLPAAT2 [symbol] PmATP:PmHXT1 [symbol] ScarMEL-PmPGK::SAD2Bex |
| D4519 | S8174 | SAD2-1vD::CavigLPAAT1 [symbol] PmATP:PmHXT1 [symbol] ScarMEL-PmPGK::SAD2Bex |
| D4690 | S8174 | SAD2-1vD::CuPSR23 LPAAT2 [symbol] 1 [symbol] PmATP:PmHXT1 [symbol] ScarMEL-PmPGK::SAD2Bex |
| D4728 | S8174 | SAD2-1vD::CkoeLPAAT [symbol] PmATP:PmHXT1 [symbol] ScarMEL-PmPGK::SAD2Bex |
| D4729 | S8174 | SAD2-1vD::CkoeLPAAT2 [symbol] PmATP:PmHXT1 [symbol] ScarMEL-PmPGK::SAD2Bex |
| D4730 | S8174 | SAD2-1vD::CprocLPAAT2 [symbol] PmATP:PmHXT1 [symbol] ScarMEL-PmPGK::SAD2Bex |
| D4551/D5683 | S8174 | SAD2-1vD::CavigGPAT9 [symbol] PmATP:PmHXT1 [symbol] ScarMEL-PmPGK::SAD2Bex |
| D4552/D4684 | S8174 | SAD2-1vD::ChookGPAT9-1-PmATP:PmHXT1 [symbol] ScarMEL-PmPGK::SAD2Bex |
| D4553/D4685 | S8174 | SAD2-1vD::CignGPAT9-1-PmATP:PmHXT1 [symbol] ScarMEL-PmPGK::SAD2Bex |
| D4554/D4686 | S8174 | SAD2-1vD::CignGPAT9-2-PmATP:PmHXT1 [symbol] ScarMEL-PmPGK::SAD2Bex |
| D4724 | S8174 | SAD2-1vD:: [symbol] GPAT9-1-PmATP:PmHXT1 [symbol] ScarMEL-PmPGK::SAD2Bex |
| D4725 | S8174 | SAD2-1vD:: [symbol] GPAT9-2-PmATP:PmHXT1 [symbol] ScarMEL-PmPGK::SAD2Bex |
| D4549 | S8174 | SAD2-1vD::CavigDGAT1 [symbol] PmATP:PmHXT1 [symbol] ScarMEL-PmPGK::SAD2Bex |
| D4681 | S8174 | SAD2-1vD::CavigDGAT1 [symbol] PmATP:PmHXT1 [symbol] ScarMEL-PmPGK::SAD2Bex |
| D4556/D4688 | S8174 | SAD2-1vD::CavigLPCAT [symbol] PmATP:PmHXT1 [symbol] ScarMEL-PmPGK::SAD2Bex |
| D4726 | S8174 | SAD2-1vD:: [symbol] LPCAT-PmATP:PmHXT1 [symbol] ScarMEL-PmPGK::SAD2Bex |
| D4556/D4689 | S8174 | SAD2-1vD::CpauLPCAT-PmATP:PmHXT1 [symbol] ScarMEL-PmPGK::SAD2Bex |
| D4727 | S8174 | SAD2-1vD::CschuLPCAT-PmATP:PmHXT1 [symbol] ScarMEL-PmPGK::SAD2Bex |
| D4732 | S8174 | SAD2-1vD::CavigPLA2-1-PmATP:PmHXT1 [symbol] ScarMEL-PmPGK::SAD2Bex |
| D4734 | S8174 | SAD2-1vD::CignPLA2-1-PmATP:PmHXT1 [symbol] ScarMEL-PmPGK::SAD2Bex |
| D4735 | S8174 | SAD2-1vD::CuPSR23PLA2-2-PmATP:PmHXT1 [symbol] ScarMEL-PmPGK::SAD2Bex |
| D4736 | S8174 | SAD2-1vD::CprocPLA2-2-PmATP:PmHXT1 [symbol] ScarMEL-PmPGK::SAD2Bex |

[symbol]

indicates data missing or illegible when filed

All the constructs shown in Table 15 can be written as SAD2-1vD::gene of interest-PmATP-PmHXT1-ScarMEL-PmPGK::SAD2B, and were made to target the transforming DNA to the SAD2 locus on the genome, thereby disrupting the expression of at least one allele of the endogenous stearoyl ACP desaturase. Sequences of all the transforming DNAs are provided below. The relevant restriction sites in the construct from 5′-3′ are- Pme I, BspQ I, Kpn I, Xho I, Avr II, Spe I, SnaB I, EcoR V, Sac I, BspQ I, Pme I respectively are indicated in lowercase, bold, and underlined. Pme I sites delimit the 5′ and 3′ ends of the transforming DNA. Bold, lowercase sequences at the 5′ and 3′ end of the construct represent genomic DNA from UTEX 1435 that target integration to the SAD2 locus via homologous recombination, wherein the SAD2 5′ flank provides the promoter for the gene of interest downstream. The primary construct was made with the previously characterized CnLPAAT gene as shown below and all other constructs were made by replacing the CnLPAAT gene with other genes of interest using the restriction sites, Kpn I and Xho I that span the gene on either side. Proceeding in the 5′ to 3′ direction, the first cassette has the codon optimized Cocos nucifera LPAAT and the Prototheca moriformis ATP synthase (PmATP) gene 3′ UTR. The initiator ATG and terminator TGA for cDNAs are indicated by uppercase italics, while the coding region is indicated with lowercase italics. The 3′ UTR is indicated by lowercase underlined text. The second cassette containing the selection gene melibiose from Saccharomyces carlsbergensis (ScarMEL1) is driven by the endogenous HXT1 promoter, and has the endogenous phosphoglycerate kinase (PmPGK) gene 3′ UTR. In this cassette, the PmHXT1 promoter is indicated by lowercase, boxed text. The initiator ATG and terminator TGA for the ScarMEL1 gene are indicated in uppercase italics, while the coding region is indicated by lowercase italics. The 3′ UTR is indicated by lowercase underlined text. All the final constructs were sequenced to ensure correct reading frames and targeting sequences.

pSZX61 Sequence of the transforming DNA expressing
CnLPAAT downstream of the SAD2 promoter in the cassette followed by the ScarMEL1
gene for selection downstream of the PmHXT1 promoter in the second cassette.
SEQ ID NO: 97
gtttaaacgccggtcaccacccgcatgctcgtactacagcgcacgcaccgcttcgtgatccaccgggtgaacgtagtcctcgacgg
aaacatctggttcgggcctcctgcttgcactcccgcccatgccgacaacctttctgctgttaccacgacccacaatgcaacgcgaca
cgaccgtgtgggactgatcggttcactgcacctgcatgcaattgtcacaagcgcttactccaattgtattcgtttgttttctgggagc
agttgctcgaccgcccgcgtcccgcaggcagcgatgacgtgtgcgtggcctgggtgtttcgtcgaaaggccagcaaccctaaatcg
caggcgatccggagattgggatctgatccgagtttggaccagatccgccccgatgcggcacgggaactgcatcgactcggcgcgg
aacccagctttcgtaaatgccagattggtgtccgatacctggatttgccatcagcgaaacaagacttcagcagcgagcgtatttgg
cgggcgtgctaccagggttgcatacattgcccatttctgtctggaccgctttactggcgcagagggtgagttgatggggttggcagg
catcgaaacgcgcgtgcatggtgtgcgtgtctgttttcggctgcacgaattcaatagtcggatgggcgacggtagaattgggtgtg
gcgctcgcgtgcatgcctcgccccgtcgggtgtcatgaccgggactggaatcccccctcgcgaccatcttgctaacgctcccgactc
tcccgaccgcgcgcaggatagactcttgttcaaccaatcgacaggtaccATGgacgcctccggcgcctcctccttcctgcgcggccgct
gcctggagtcctgcttcaaggcctccttcggctacgtaatgtcccagcccaaggacgccgccggccagccctcccgccgccccgccgacgcc
gacgacttcgtggacgacgaccgctggatcaccgtgatcctgtccgtggtgcgcatcgccgcctgcttcctgtccatgatggtgaccaccatc
gtgtggaacatgatcatgctgatcctgctgccctggccctacgcccgcatccgccagggcaacctgtacggccacgtgaccggccgcatgct
gatgtggattctgggcaaccccatcaccatcgagggctccgagttctccaacacccgcgccatctacatctgcaaccacgcctccctggtgg
acatcttcctgatcatgtggctgatccccaagggcaccgtgaccatcgccaagaaggagatcatctggtatcccctgttcggccagctgtac
gtgctggccaaccaccagcgcatcgaccgctccaacccctccgccgccatcgagtccatcaaggaggtggcccgcgccgtggtgaagaag
aacctgtccctgatcatcttccccgagggcacccgctccaagaccggccgcctgctgcccttcaagaagggcttcatccacatcgccctccag
acccgcctgcccatcgtgccgatggtgctgaccggcacccacctggcctggcgcaagaactccctgcgcgtgcgccccgcccccatcaccgt
gaagtacttctcccccatcaagaccgacgactgggaggaggagaagatcaaccactacgtggagatgatccacgccctgtacgtggacc
acctgcccgagtcccagaagcccctggtgtccaagggccgcgacgcctccggccgctccaactccTGAttaattaactcgagatgtggaga
tgtagggtggtcgactcgttggaggtgggtgtttttttttatcgagtgcgcggcgcggcaaacgggtccctttttatcgaggtgttccca
acgccgcaccgccctcttaaaacaacccccaccaccacttgtcgaccttctcgtttgttatccgccacggcgccccggaggggcgtcg
tctggccgcgcgggcagctgtatcgccgcgctcgctccaatggtgtgtaatcttggaaagataataatcgatggatgaggaggaga
gcgtgggagatcagagcaaggaatatacagttggcacgaagcagcagcgtactaagctgtagcgtgttaagaaagaaaaactcg

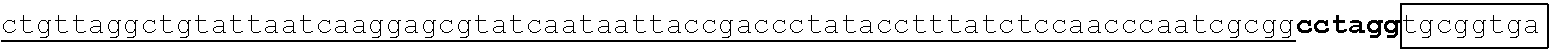

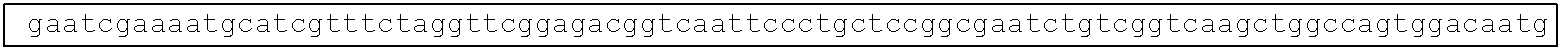

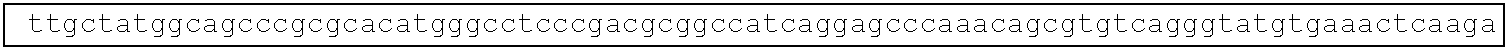

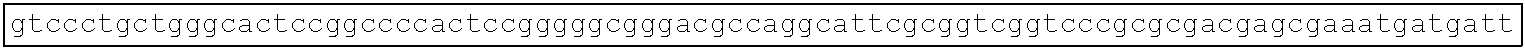

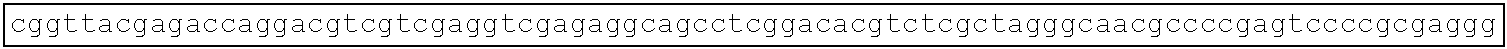

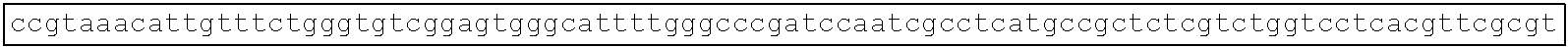

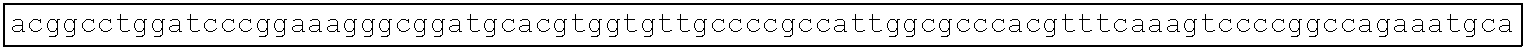

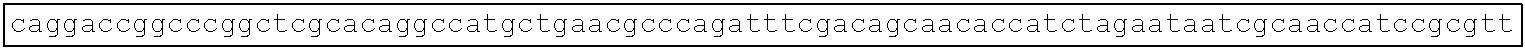

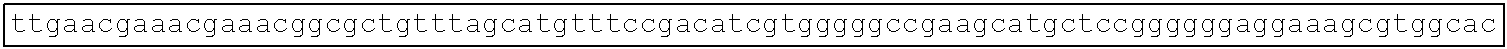

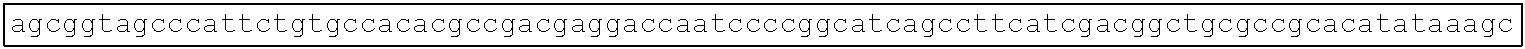

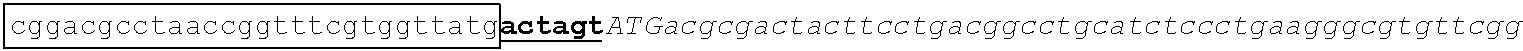

cgtctccccctcctacaacggcctgggcctgacgccccagatgggctgggacaactggaacacgttcgcctgcgacgtctccga
gcagctgctgctggacacggccgaccgcatctccgacctgggcctgaaggacatgggctacaagtacatcatcctggacgact
gctggtcctccggccgcgactccgacggcttcctggtcgccgacgagcagaagaccccaacggcatgggccacgtcgccgac
cacctgcacaacaactccttcctgacggcatgtactcctccgcgggcgagtacacgtgcgccggctaccccggctccctgggcc
gcgaggaggaggacgcccagttcttcgcgaacaaccgcgtggactacctgaagtacgacaactgctacaacaagggccagt
tcggcacgcccgagatctcctaccaccgctacaaggccatgtccgacgccctgaacaagacgggccgccccatcttctactccct
gtgcaactggggccaggacctgaccttctactggggctccggcatcgcgaactcctggcgcatgtccggcgacgtcacggcgg
agttcacgcgccccgactcccgctgcccctgcgacggcgacgagtacgactgcaagtacgccggcttccactgctccatcatga
acatcctgaacaaggccgcccccatgggccagaacgcgggcgtcggcggctggaacgacctggacaacctggaggtcggcg
tcggcaacctgacggacgacgaggagaaggcgcacttctccatgtgggccatggtgaagtcccccctgatcatcggcgcgaa
cgtgaacaacctgaaggcctcctcctactccatctactcccaggcgtccgtcatcgccatcaaccaggactccaacggcatcccc
gccacgcgcgtctggcgctactacgtgtccgacacggacgagtacggccagggcgagatccagatgtggtccggccccctgg
acaacggcgaccaggtcgtggcgctgctgaacggcggctccgtgtcccgccccatgaacacgaccctggaggagatcttcttc
gactccaacctgggctccaagaagctgacctccacctgggacatctacgacctgtgggcgaaccgcgtcgacaactccacggc
gtccgccatcctgggccgcaacaagaccgccaccggcatcctgtacaacgccaccgagcagtcctacaaggacggcctgtcca
agaacgacacccgcctgttcggccagaagatcggctccctgtcccccaacgcgatcctgaacacgaccgtccccgcccacggc
atcgcgttctaccgcctgcgcccctcctccTGAtacaacttattacgtattctgaccggcgctgatgtggcgcggacgccgtcgtac
tctttcagactttactcttgaggaattgaacctttctcgcttgctggcatgtaaacattggcgcaattaattgtgtgatgaagaaaggg
tggcacaagatggatcgcgaatgtacgagatcgacaacgatggtgattgttatgaggggccaaacctggctcaatcttgtcgcatgt
ccggcgcaatgtgatccagcggcgtgactctcgcaacctggtagtgtgtgcgcaccgggtcgctttgattaaaactgatcgcattgcc
atcccgtcaactcacaagcctactctagctcccattgcgcactcgggcgcccggctcgatcaatgttctgagcggagggcgaagcgt
caggaaatcgtctcggcagctggaagcgcatggaatgcggagcggagatcgaatcagatatcAAGCTCCATCgagctccagc
cacggcaacaccgcgcgccttgcggccgagcacggcgacaagaacctgagcaagatctgcgggctgatcgccagcgacgaggg
ccggcacgagatcgcctacacgcgcatcgtggacgagttcttccgcctcgaccccgagggcgccgtcgccgcctacgccaacatga
tgcgcaagcagatcaccatgcccgcgcacctcatggacgacatgggccacggcgaggccaacccgggccgcaacctcttcgccga
cttctccgcggtcgccgagaagatcgacgtctacgacgccgaggactactgccgcatcctggagcacctcaacgcgcgctggaag
gtggacgagcgccaggtcagcggccaggccgccgcggaccaggagtacgtcctgggcctgccccagcgcttccggaaactcgcc
gagaagaccgccgccaagcgcaagcgcgtcgcgcgcaggcccgtcgccttctcctggatctccgggcgcgagatcatggtctagg
gagcgacgagtgtgcgtgcggggctggcgggagtgggacgccctcctcgctcctctctgttctgaacggaacaatcggccaccccg
cgctacgcgccacgcatcgagcaacgaagaaaaccccccgatgataggttgcggtggctgccgggatatagatccggccgcaca
tcaaagggcccctccgccagagaagaagctcctttcccagcagactcctgaagagcgtttaaac.

The sequence for all of the other acyltransferase constructs are identical to that of pSZEX61 with the exception of the encoded acyltransferase. The acyltransferase sequence alone is provided below for the remaining acyltransferase constructs.

SEQ ID NO: 98 CpauLPAAT1
ggtaccATGgccatccccgccgccgccgtgatcttcctgttcggcctgctgttcttcacctccggcctgatcatcaacctgttccagg
ccctgtgcttcgtgctggtgtggcccctgtccaagaacgcctaccgccgcatcaaccgcgtgttcgccgagctgctgctgtccgagc
tgctgtgcctgttcgactggtgggccggcgccaagctgaagctgttcaccgaccccgagaccttccgcctgatgggcaaggagca
cgccctggtgatcatcaaccacatgaccgagctggactggatgctgggctgggtgatgggccagcacctgggctgcctgggctcc
atcctgtccgtggccaagaagtccaccaagttcctgcccgtgctgggctggtccatgtggttctccgagtacctgtacatcgagcgct
cctgggccaaggaccgcaccaccctgaagtcccacatcgagcgcctgaccgactaccccctgcccttctggatggtgatcttcgtg
gagggcacccgcttcacccgcaccaagctgctggccgcccagcagtacgccgcctcctccggcctgcccgtgccccgcaacgtg
ctgatcccccgcaccaagggcttcgtgtcctgcgtgtcccacatgcgctccttcgtgcccgccgtgtacgacgtgaccgtggccttcc
ccaagacctcccccccccccaccctgctgaacctgttcgagggccagtccatcgtgctgcacgtgcacatcaagcgccacgccat
gaaggacctgcccgagtccgacgacgccgtggcccagtggtgccgcgacaagttcgtggagaaggacgccctgctggacaag
cacaacgccgaggacaccttctccggccaggaggtgcaccgcaccggctcccgccccatcaagtccctgctggtggtgatctcct
gggtggtggtgatcaccttcggcgccctgaagttcctgcagtggtcctcctggaagggcaaggccttctccgtgatcggcctgggc
atcgtgaccctgctgatgcacatgctgatcctgtcctcccaggccgagcgctcctccaaccccgccaaggtggcccaggccaagc
tgaagaccgagctgtccatctccaagaaggccaccgacaaggagaacTGActcgag
SEQ ID NO: 99 CprocLPAAT1
ggtacc

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ctcgag
SEQ ID NO: 100 CpaiLPAAT1
ggtaccATGgccatcccctccgccgccgtggtgacctgacggcctgctgacttcacctccggcctgatcatcaacctgaccagg
ccactgcacgtgctgatctcccccctgtccaagaacgcctaccgccgcatcaaccgcgtgacgccgagctgctgcccctggagtt
cctgtggctgaccactggtgcgccggcgccaagctgaagctgacaccgaccccgagaccaccgcctgatgggcaaggagcac
gccctggtgatcatcaaccacaagatcgagctggactggatggtgggctgggtgctgggccagcacctgggctgcctgggctcca
tcctgtccgtggccaagaagtccaccaagacctgcccgtgacggctggtccctgtggactccggctacctgacctggagcgctcc
tgggccaaggacaagatcaccctgaagtcccacatcgagtccctgaaggactaccccctgccataggctgatcatcacgtgga
gggcacccgatcacccgcaccaagctgctggccgcccagcagtacgccgcctcctccggcctgcccgtgccccgcaacgtgct
gatcccccacaccaagggcttcgtgtcctccgtgtcccacatgcgctccttcgtgcccgccatctacgacgtgaccgtggccttcccc
aagacctcccccccccccaccatgctgaagctgacgagggccagtccgtggagctgcacgtgcacatcaagcgccacgccatg
aaggacctgcccgagtccgacgacgccgtggcccagtggtgccgcgacaagacgtggagaaggacgccctgctggacaagc
acaactccgaggacaccactccggccaggaggtgcaccacgtgggccgccccatcaaggccctgctggtggtgatctcctgggt
ggtggtgatcatcacggcgccctgaagacctgctgtggtcctccctgctgtcctcctggaagggcaaggccactccgtgatcggcc
tgggcatcgtggccggcatcgtgaccctgctgatgcacatcctgatcctgtcctcccaggccgagggctccaaccccgtgaaggc
cgcccccgccaagctgaagaccgagctgtcctcctccaagaaggtgaccaacaaggagaacTGActcgag
SEQ ID NO: 101 ChookLPAAT1
ggtaccATGgccatcccctccgccgccgtggtgacctgacggcctgctgacttcacctccggcctgatcatcaacctgaccagg
ccactgatcgtgctgatctcccccctgtccaagaacgcctaccgccgcatcaaccgcgtgacgccgagctgctgcccctggagtt
cctgtggctgaccactggtgcgccggcgccaagctgaagctgacaccgaccccgagaccaccgcctgatgggcaaggagcac
gccctggtgatcatcaaccacaagatcgagctggactggatggtgggctgggtgctgggccagcacctgggctgcctgggctcca
tcctgtccgtggccaagaagtccaccaagacctgcccgtgacggctggtccctgtggactccgagtacctgacctggagcgctcc
tgggccaaggacaagatcaccctgaagtcccacatcgagtccctgaaggactaccccctgccataggctgatcatcacgtgga
gggcacccgatcacccgcaccaagctgctggccgcccagcagtacgccgcctcctccggcctgcccgtgccccgcaacgtgct
gatcccccacaccaagggcttcgtgtcctccgtgtcccacatgcgctccttcgtgcccgccatctacgacgtgaccgtggccttcccc
aagacctcccccccccccaccatgctgaagctgacgagggccagtccgtggagctgcacgtgcacatcaagcgccacgccatg
aaggacctgcccgagtccgacgacgccgtggcccagtggtgccgcgacaagacgtggagaaggacgccctgctggacaagc
acaactccgaggacaccactccggccaggaggtgcaccacgtgggccgccccatcaaggccctgctggtggtgatctcctgggt
ggtggtgatcatcacggcgccctgaagacctgctgtggtcctccctgctgtcctcctggaagggcaaggccactccgtgatcggcc
tgggcatcgtggccggcatcgtgaccctgctgatgcacatcctgatcctgtcctcccaggccgagggctccaaccccgtgaaggc
cgcccccgccaagctgaagaccgagctgtcctcctccaagaaggtgaccaacaaggagaacTGActcgag
SEQ ID NO: 102 CignLPAAT1
ggtaccATGgccatcgccgccgccgccgtgatcacctgacggcctgctgacttcgcctccggcatcatcatcaacctgaccag
gccctgtgcttcgtgctgatctggcccctgtccaagaacgtgtaccgccgcatcaaccgcgtgttcgccgagctgctgctgatggac
ctgctgtgcctgaccactggtgggccggcgccaagatcaagctgacaccgaccccgagaccaccgcctgatgggcatggagca
cgccctggtgatcatgaaccacaagaccgacctggactggatggtgggctggatcctgggccagcacctgggctgcctgggctc
catcctgtccatcgccaagaagtccaccaagacatccccgtgctgggctggtccgtgtggactccgagtacctgacctggagcgc
tcctgggccaaggacaagtccaccctgaagtcccacatggagaagctgaaggactaccccctgccataggctggtgatcacgt
ggagggcacccgatcacccgcaccaagctgctggccgcccagcagtacgccgcctcctccggcctgcccgtgccccgcaacgt
gctgatcccccacaccaagggcttcgtgtcctgcgtgtccaacatgcgctccttcgtgcccgccgtgtacgacgtgaccgtggcctt
ccccaagtcctcccccccccccaccatgctgaagctgacgagggccagtccatcgtgctgcacgtgcacatcaagcgccacgcc
ctgaaggacctgcccgagtccgacgacgccgtggcccagtggtgccgcgacaagacgtggagaaggacgccctgctggacaa
gcacaacgccgaggacaccactccggccaggaggtgcaccacatcggccgccccatcaagtccctgctggtggtgatcgcctg
ggtggtggtgatcatcacggcgccctgaagacctgcagtggtcctccctgctgtccacctggaagggcaaggccactccgtgatc
ggcctgggcatcgccaccctgctgatgcacatgctgatcctgtcctcccaggccgagcgctccaaccccgccaaggtggccaag
TGActcgag
SEQ ID NO: 103 CavigLPAAT1
ggtaccATGaccatcgcctccgccgccgtggtgttcctgttcggcatcctgctgttcacctccggcctgatcatcaacctgttccag
gccttctgctccgtgctggtgtggcccctgtccaagaacgcctaccgccgcatcaaccgcgtgttcgccgagttcctgcccctggag
ttcctgtggctgttccactggtgggccggcgccaagctgaagctgttcaccgaccccgagaccttccgcctgatgggcaaggagc
acgccctggtgatcatcaaccacaagatcgagctggactggatggtgggctgggtgctgggccagcacctgggctgcctgggctc
catcctgtccgtggccaagaagtccaccaagacctgcccgtgttcggctggtccctgtggttctccgagtacctgttcctggagcgc
aactgggccaaggacaagaagaccctgaagtcccacatcgagcgcctgaaggactaccccctgcccttctggctgatcatcttcg
tggagggcacccgcttcacccgcaccaagctgctggccgcccagcagtacgccgcctccgccggcctgcccgtgccccgcaac
gtgctgatcccccacaccaagggatcgtgtcctccgtgtcccacatgcgctccacgtgcccgccatctacgacgtgaccgtggcct
tccccaagacctcccccccccccaccatgctgaagctgttcgagggccacttcgtggagctgcacgtgcacatcaagcgccacgc
catgaaggacctgcccgagtccgaggacgccgtggcccagtggtgccgcgacaagttcgtggagaaggacgccctgctggac
aagcacaacgccgaggacaccttctccggccaggaggtgcaccacgtgggccgccccatcaagtccctgctggtggtgatctcc
tgggtggtggtgatcatcttcggcgccctgaagttcctgcagtggtcctccctgctgtcctcctggaagggcatcgccttctccgtgat
cggcctgggcaccgtggccctgctgatgcagatcctgatcctgtcctcccaggccgagcgctccatccccgccaaggagaccccc
gccaacctgaagaccgagctgtcctcctccaagaaggtgaccaacaaggagaacTGActcgag
SEQ ID NO: 104 CavigLPAAT2
ggtaccATGgccatcgccgccgccgccgtgatcgtgcccgtgtccctgctgttcttcgtgtccggcctgatcgtgaacctggtgca
ggccgtgtgcttcgtgctgatccgccccctgttcaagaacacctaccgccgcatcaaccgcgtggtggccgagctgctgtggctgg
agctggtgtggctgatcgactggtgggccggcgtgaagatcaaggtgttcaccgaccacgagaccttccacctgatgggcaagg
agcacgccctggtgatctgcaaccacaagtccgacatcgactggctggtgggctgggtgctggcccagcgctccggctgcctggg
ctccaccctggccgtgatgaagaagtcctccaagttcctgcccgtgatcggctggtccatgtggactccgagtacctgttcctggag
cgcaactgggccaaggacgagtccaccctgaagtccggcctgaaccgcctgaaggactaccccctgcccttctggctggccctgt
tcgtggagggcacccgcttcacccgcgccaagctgctggccgcccagcagtacgccgcctcctccggcctgcccgtgccccgca
acgtgctgatcccccgcaccaagggatcgtgtcctccgtgtcccacatgcgctcatcgtgcccgccatctacgacgtgaccgtgg
ccatccccaagacctcccccccccccaccctgctgcgcatgttcaagggccagtcctccgtgctgcacgtgcacctgaagcgcca
ccagatgaacgacctgcccgagtccgacgacgccgtggcccagtggtgccgcgacatcttcgtggagaaggacgccctgctgg
acaagcacaacgccgaggacaccttctccggccaggagctgcaggacaccggccgccccatcaagtccctgctgatcgtgatct
cctgggccgtgctggtggtgttcggcgccgtgaagttcctgcagtggtcctccctgctgtcctcctggaagggcctggccttctccgg
catcggcctgggcgtgatcaccctgctgatgcacatcctgatcctgttctcccagtccgagcgctccacccccgccaaggtggccc
ccgccaagcccaagatcgagggcgagtcctccaagaccgagatggagaaggagcacTGActcgag
SEQ ID NO: 105 CpalLPAAT1
ggtaccATGgccatcgccgccgccgccgtgatcgtgcccctgggcctgctgttcttcgtgtccggcctgatcgtgaacctggtgca
ggccgtgtgcttcgtgctgatccgccccctgtccaagaacacctaccgccgcatcaaccgcgtggtggccgagctgctgtggctgg
agctggtgtggctgatcgactggtgggccggcgtgaagatcaaggtgttcaccgaccacgagaccctgtccctgatgggcaagg
agcacgccctggtgatctgcaaccacaagtccgacatcgactggctggtgggctgggtgctggcccagcgctccggctgcctggg
ctccaccctggccgtgatgaagaagtcctccaagttcctgcccgtgatcggctggtccatgtggttctccgagtacctgcccgagtcc
gacgacgccgtggcccagtggtgccgcgacatcttcgtggagaaggacgccctgctggacaagcacaacgccgaggacacctt
ctccggccaggagctgcaggacaccggccgccccatcaagtccctgctggtggtgatctcctgggccgtgctggtgatcttcggcg
ccgtgaagttcctgcagtggtcctccctgctgtcctcctggaagggcctggccttctccggcgtgggcctgggcatcatcaccctgct
gatgcacatcctgatcctgttctcccagtccgagcgctccacccccgccaaggtggcccccgccaagcccaagaaggacggcga
gtcctccaagaccgagatcgagaaggagaacgttcctggagcgctcctgggccaaggacgagaacaccctgaagtccggcct
gaaccgcctgaaggactaccccctgcccttctggctggccctgttcgtggagggcacccgcttcacccgcgccaagctgctggcc
gcccagcagtacgccacctcctccggcctgcccgtgccccgcaacgtgctgatcccccgcaccaagggcttcgtgtcctccgtgtc
ccacatgcgctcatcgtgcccgccatctacgacgtgaccgtggccatccccaagacctcccccccccccaccatgctgcgcatgtt
caagggccagtcctccgtgctgcacgtgcacctgaagcgccacctgatgaaggacctTGActcgag
SEQ ID NO: 106 CuPSR23 LPAAT2
ggtaccATGgccatcgccgccgccgccgtgatcacctgttcggcctgatatatcgcctccggcctgatcatcaacctgttccag
gccctgtgcttcgtgctgatccgccccctgtccaagaacgcctaccgccgcatcaaccgcgtgttcgccgagctgctgctgtccgag
ctgctgtgcctgacgactggtgggccggcgccaagctgaagctgttcaccgaccccgagaccaccgcctgatgggcaaggagc
acgccctggtgatcatcaaccacatgaccgagctggactggatggtgggctgggtgatgggccagcacttcggctgcctgggctc
catcatctccgtggccaagaagtccaccaagacctgcccgtgctgggctggtccatgtggactccgagtacctgtacctggagcg
ctcctgggccaaggacaagtccaccctgaagtcccacatcgagcgcctgatcgactaccccctgcccactggctggtgatcacgt
ggagggcacccgcttcacccgcaccaagctgctggccgcccagcagtacgccgtgtcctccggcctgcccgtgccccgcaacgt
gctgatcccccgcaccaagggcacgtgtcctgcgtgtcccacatgcgctccacgtgcccgccgtgtacgacgtgaccgtggccac
cccaagacctcccccccccccaccctgctgaacctgacgagggccagtccatcatgctgcacgtgcacatcaagcgccacgcca
tgaaggacctgcccgagtccgacgacgccgtggccgagtggtgccgcgacaagacgtggagaaggacgccctgctggacaa
gcacaacgccgaggacaccactccggccaggaggtgtgccactccggctcccgccagctgaagtccctgctggtggtgatctcc
tgggtggtggtgaccaccttcggcgccctgaagacctgcagtggtcctcctggaagggcaaggccactccgccatcggcctggg
catcgtgaccctgctgatgcacgtgctgatcctgtcctcccaggccgagcgctccaaccccgccgaggtggcccaggccaagctg
aagaccggcctgtccatctccaagaaggtgaccgacaaggagaacTGActcgag
SEQ ID NO: 107 CkoeLPAAT1
ggtaccATGgccatccccgccgccgtggccgtgatccccatcggcctgctgacatcatctccggcctgatcgtgaacctgatcca
ggccgtggtgtacgtgctgatccgccccctgtccaagaacctgcaccgcaagatcaacaagcccatcgccgagctgctgtggctg
gagctgatctggctggtggactggtgggccggcatcaaggtggaggtgtacgccgactcccagaccctggagctgatgggcaag
gagcacgccctgctgatctgcaaccaccgctccgacatcgactggctggtgggctgggtgctggcccagcgcgcccgctgcctgg
gctccgccctggccatcatgaagaagtccgccaagttcctgcccgtgatcggctggtccatgtggactccgactacatcacctgga
ccgcacctgggccaaggacgagaagaccctgaagtccggatcgagcgcctggccgacttccccatgccatctggctggccctg
acgtggagggcacccgatcaccaaggccaagctgctggccgcccaggagtacgccgcctcccgcggcctgcccgtgccccag
aacgtgctgatcccccgcaccaagggatcgtgaccgccgtgacccacatgcgctcctacgtgcccgccatctacgactgcaccg
tggacatctccaaggcccaccccgccccctccatcctgcgcctgatccgcggccagtcctccgtggtgaaggtgcagatcacccg
ccactccatgcaggagctgcccgagaccgccgacggcatctcccagtggtgcatggacctgttcgtgaccaaggacggcacctg
gagaagtaccactccaaggacatcacggctccctgcccgtgcagaacatcggccgccccgtgaagtccctgatcgtggtgctgtg
ctggtactgcctgatggccacggcctgacaagacttcatgtggtcctccctgctgtcctcctgggagggcatcctgtccctgggcctg
atcctgctggccgtggccatcgtgatgcagatcctgatccagtccaccgagtccgagcgctccacccccgtgaagtccatccaga
aggacccctccaaggagaccctgctgcagaacTGActcgag
SEQ ID NO: 108 CkoeLPAAT2
ggtaccATGcacgtgctgctggagatggtgaccaccgcactcctccacttcgtgacgacaacgtgcaggccctgtgatcgtgct
gatctggcccctgtccaagtccgcctaccgcaagatcaaccgcgtgacgccgagctgctgctgtccgagctgctgtgcctgacga
ctggtgggccggcgccaagctgaagctgacaccgaccccgagaccaccgcctgatgggcaaggagcacgccctggtgatcac
caaccacaagatcgacctggactggatgatcggctggatcctgggccagcacttcggctgcctgggctccgtgatctccatcgcca
agaagtccaccaagacctgcccatcacggctggtccctgtggactccgagtacctgacctggagcgcaactgggccaaggaca
agcgcaccctgaagtcccacatcgagcgcatgaaggactaccccctgcccctgtggctgatcctgacgtggagggcacccgat
cacccgcaccaagctgctggccgcccagcagtacgccgcctcctccggcctgcccgtgccccgcaacgtgctgatcccccacac
caagggcttcgtgtcctccgtgtcccacatgcgctccttcgtgcccgccgtgtacgacgtgaccgtggccttccccaagacctcccc
cccccccaccatgctgtccctgacgagggccagtccgtggtgctgcacgtgcacatcaagcgccacgccatgaaggacctgccc
gactccgacgacgccgtggcccagtggtgccgcgacaagacgtggagaaggacgccctgctggacaagcacaacgccgagg
acaccactccggccaggaggtgcaccacgtgggccgccccatcaagtccctgctggtggtgatctcctggatggtggtgatcatct
tcggcgccctgaagacctgcagtggtcctccctgctgtcctcctggaagggcaaggccactccgccatcggcctgggcatcgcca
ccctgctgatgcacgtgctggtggtgactcccaggccgaccgctccaaccccgccaaggtgccccccgccaagctgaacaccga
gctgtcctcctccaagaaggtgaccaacaaggagaacTGActcgag
SEQ ID NO: 109 CprocLPAAT2
ggtaccATGgccatccccgccgccgtggccgtgatccccatcggcctgctgacatcatctccggcctgatcgtgaacctgatcca
ggccgtggtgtacgtgctgatccgccccctgtccaagaacctgtaccgcaagatcaacaagcccatcgccgagctgctgtggctg
gagctgatctggctggtggactggtgggccggcatcaaggtggaggtgtacgccgactccgagaccctggagtccatgggcaag
gagcacgccctgctgatctgcaaccaccgctccgacatcgactggctggtgggctgggtgctggcccagcgcgcccgctgcctgg
gctccgccctggccatcatgaagaagtccgccaagttcctgcccgtgatcggctggtccatgtggttctccgactacatcttcctgga
ccgcacctgggagaaggacgagaagaccctgaagtccggcttcgagcgcctggccgacttccccatgcccttctggctggccct
gttcgtggagggcacccgcttcaccaaggccaagctgctggccgcccaggagttcgccgcctcccgcggcctgcccgtgcccca
gaacgtgctgatcccccgcaccaagggcttcgtgaccgccgtgacccacatgcgctcctacgtgcccgccatctacgactgcacc
gtggacatctccaaggcccaccccgccccctccatcctgcgcctgatccgcggccagtcctccgtggtgaaggtgcagatcaccc
gccactccatgcaggagctgcccgagacccccgacggcatctcccagtggtgcatggacctgttcgtgaccaaggacgccttcct
ggagaagtaccactccaaggacatcttcggctccctgcccgtgcacgacatcggccgccccgtgaagtccctgatcgtggtgctgt
gctggtactccctgatggccttcggcactacaagttcttcatgtggtcctccctgctgtcctcctgggagggcatcctgtccctgggcct
ggtgctgatcgtgatcgccatcgtgatgcagatcctgatccagtcctccgagtccgagcgctccacccccgtgaagtccgtgcaga
aggacccctccaaggagaccctgctgcagaacTGActcgag
SEQ ID NO: 110 CavigGPAT9
ggtaccATGgccaccggcggctccctgaagccctcctcctccgacctggacctggaccaccccaacatcgaggactacctgcc
ctccggctcctccatcaacgagcccgccggcaagctgcgcctgcgcgacctgctggacatctcccccaccctgaccgaggccgc
cggcgccatcgtggacgactccttcacccgctgatcaagtccatcccccgcgagccctggaactggaacctgtacctgttccccct
gtggtgcatcggcgtgctgatccgctacttcatcctgttccccggccgcgtgatcgtgctgaccatgggctggatcaccgtgatctcct
catcatcgccgtgcgcgtgctgctgaagggccacgacgccctgcagatcaagctggagcgcctgatcgtgcagctgctgtgctcc
tcatcgtggcctcctggaccggcgtggtgaagtaccacggcccccgcccctccatccgccccaagcaggtgtacgtggccaacc
acacctccatgatcgacttcttcatcctggaccagatgaccgtgttctccgtgatcatgcagaagcaccccggctgggtgggcctgc
tgcagtccaccctgctggagtccgtgggctgcatctggacgaccgcgccgaggccaaggaccgcggcatcgtggccaagaagc
tgtgggaccacgtgcacggcgagggcaacaaccccctgctgatcttccccgagggcacctgcgtgaacaacaactactccgtga
tgttcaagaagggcgccttcgagctgggctgcaccgtgtgccccgtggccatcaagtacaacaagatcttcgtggacgccttctgg
aactccaagaagcagtccttcacccgccacctgctgcagctgatgacctcctgggccgtggtgtgcgacgtgtggtacttggagcc
ccagaccctgaagcccggcgagacccccatcgagttcgccgagcgcgtgcgcgacatcatctccgcccgcgccggcctgaaga
aggtgccctgggacggctacctgaagtactcccgcccctcccccaagcaccgcgagcgcaagcagcagaccttcgccgagtcc
gtgctgcagcgcctggaggagTGActcgag
SEQ ID NO: 111 ChookGPAT9-1
ggtaccATGgccaccgccggctccctgaagccctcccgctccgagctggacttcgaccgccccaacatcgaggactacctgcc
ctccggctcctccatcatcgagcccgccggcaagctgcgcctgcgcgacctgctggacatctcccccaccctgaccgaggccgcc
ggcgccatcgtggacgactccttcacccgctgatcaagtccaacccccccgagccctggaactggaacatctacctgttccccct
gtggtgcttcggcgtgctgatccgctacctgatcctgttccccgcccgcgtgatcgtgctgaccatcggctggatcatcttcctgtcctc
cttcatccccgtgcacctgctgctgaagggccacgacgccctgcgcatcaagctggagcgcctgctggtggagctgatctgctcat
cttcgtggcctcctggaccggcgtggtgaagtaccacggcccccgcccctccatccgccccaagcaggtgtacgtggccaaccac
acctccatgatcgacttcttcatcctggaccagatgaccgtgttctccgtgatcatgcagaagcaccccggctgggtgggcctgctg
cagtccaccctgctggagtccgtgggctgcatctggttcgaccgcgccgaggccaaggaccgcggcatcgtggccaagaagctg
tgggaccacgtgcacggcgagggcaacaaccccctgctgatcttccccgagggcacctgcgtgaacaacaactactccgtgatg
ttcaagaagggcgccttcgagctgggctgcaccgtgtgccccgtggccatcaagtacaacaagatcttcgtggacgccttctggaa
ctccaagaagcagtccttcacccgccacctgctgcagctgatgacctcctgggccgtggtgtgcgacgtgtggtacttggagcccc
agaccctgaagcccggcgagacccccatcgagttcgccgagcgcgtgcgcgacatcatctccgtgcgcgccggcctgaagaag
gtgccctgggacggctacctgaagtactcccgcccctcccccaagcacaccgagcgcaagcagcagaacttcgccgagtccgt
gctgcagcgcctggagaagaagTGActcgag
SEQ ID NO: 112 CignGPAT9-1
ggtaccATGgccaccggcggccgcctgaagccctcctcctccgagctggacctggaccgcgccaacaccgaggactacctgc
cctccggctcctccatcaacgagcccgtgggcaagctgcgcctgcgcgacctgctggacatctcccccaccctgaccgaggccg
ccggcgccatcgtggacgactccttcacccgctgcttcaagtccatcccccccgagccctggaactggaacatctacctgttccccc
tgtggtgatcggcgtgctgatccgctacttcatcctgttccccgcccgcgtgatcgtgctgaccatcggctggatcaccgtgatctcct
catcaccgccgtgcgcacctgctgaagggccacaacgccctgcagatcaagctggagcgcctgatcgtgcagctgctgtgctcc
tcatcgtggcctcctggaccggcgtggtgaagtaccacggcccccgcccctccatccgccccaagcaggtgtacgtggccaacc
acacctccatgatcgacacctgatcctggaccagatgaccgtgactccgtgatcatgcagaagcaccccggctgggtgggcctg
ctgcagtccaccctgctggagtccgtgggctgcatctggacaaccgcgccgaggccaaggaccgcgagatcgtggccaagaag
ctgtgggaccacgtgcacggcgagggcaacaaccccctgctgatcaccccgagggcacctgcgtgaacaaccactactccgtg
atgacaagaagggcgccacgagctgggctgcaccgtgtgccccgtggccatcaagtacaacaagatcacgtggacgccactg
gaactcccgcaagcagtccacaccatgcacctgctgcagctgatgacctcctgggccgtggtgtgcgacgtgtggtacaggagc
cccagaccctgaagcccggcgagaccgccatcgagacgccgagcgcgtgcgcgacatcatctccgtgcgcgccggcctgaag
aaggtgccctgggacggctacctgaagtactcccgcccctcccccaagcaccgcgagtccaagcagcagtccacgccgagtcc
gtgctgcgccgcctggaggagaagTGActcgag
SEQ ID NO: 113 CignGPAT9-2
ggtaccATGgccaccggcggccgcctgaagccctcctcctccgagctggacctggaccgcgccaacaccgaggactacctgc
cctccggctcctccatcaacgagcccgtgggcaagctgcgcctgcgcgacctgctggacatctcccccaccctgaccgaggccg
ccggcgccatcgtggacgactccacacccgctgatcaagtccatcccccccgagccctggaactggaacatctacctgaccccc
tgtggtgatcggcgtgctgatccgctacttcatcctgaccccgcccgcgtgatcgtgctgaccatcggctggatcaccgtgatctcct
catcaccgccgtgcgcacctgctgaagggccacaacgccctgcagatcaagctggagcgcctgatcgtgcagctgctgtgctcc
tccacgtggcctcctggaccggcgtggtgaagtaccacggcccccgcccctccatccgccccaagcaggtgtacgtggccaacc
acacctccatgatcgacacctgatcctggaccagatgaccgtgactccgtgatcatgcagaagcaccccggctgggtgggcctg
ctgcagtccaccctgctggagtccgtgggctgcatctggacaaccgcgccgaggccaaggaccgcgagatcgtggccaagaag
ctgtgggaccacgtgcacggcgagggcaacaaccccctgctgatcaccccgagggcacctgcgtgaacaaccactactccgtg
atgacaagaagggcgccacgagctgggctgcaccgtgtgccccgtggccatcaagtacaacaagatcacgtggacgccactg
gaactccaagaagcactccacacccgccacctgctgcagctgatgacctcctgggccgtggtgtgcgacgtgtggtacaggagc
cccagaccctgaagcccggcgagacccccatcgagacgccgagcgcgtgcgcgacatcatctccgtgcgcgccgacctgaag
aaggtgccctgggacggctacctgaagtactcccgcccctcccccaagcaccgcgagcgcaagcagcagaagacgccgagtc
cgtgctgcgccgcctggaggagaagTGActcgag
SEQ ID NO: 114 CpalGPAT9-1
ggtaccATGgccaccgccggccgcctgaagccctcctcctccgagctggagctggacctggaccgccccaacatcgaggact
acctgccctccggctcctccatcaacgagcccgccggcaagctgcgcctgcgcgacctgctggacatctcccccatgctgaccga
ggccgccggcgccatcgtggacgactccacacccgctgatcaagtccatcccccccgagccctggaactggaacatctacctgt
tccccctgtggtgatcggcgtgctgatccgctacctgatcctgaccccgcccgcgtgatcgtgctgaccgtgggctggatcaccgtg
atctcctccacatcaccgtgcgcacctgctgaagggccacgactccctgcgcatcaagctggagcgcctgatcgtgcagctgttct
gctcctccacgtggcctcctggaccggcgtggtgaagtaccacggcccccgcccctccatccgcccccagcaggtgtacgtggcc
aaccacacctccatgatcgacttcatcatcctgaaccagatgaccgtgactccgccatcatgcagaagcaccccggctgggtggg
cctgatccagtccaccatcctggagtccgtgggctgcatctggacaaccgcgccgaggccaaggaccgcgagatcgtggccaa
gaagctgctggaccacgtgcacggcgagggcaacaaccccctgctgatcaccccgagggcacctgcgtgaacaaccactactc
cgtgatgacaagaagggcgccacgagctgggctgcaccgtgtgccccgtggccatcaagtacaacaagatcacgtggacgcct
tctggaactccaagaagcagtccacaccatgcacctgctgcagctgatgacctcctgggccgtggtgtgcgacgtgtggtacagg
agccccagaccctgaagcccggcgagacccccatcgagacgccgagcgcgtgcgcgacatcatctccgtgcgcgccggcctg
aagaaggtgccctgggacggctacctgaagtactcccgcccctcccccaagcaccgcgagcgcaagcagcagtccacgccga
gtccgtgctgcgccgcctggagaagcgcTGActcgag
SEQ ID NO: 115 CpalGPATt9-2
ggtaccATGgccaccgccggccgcctgaagccctcctcctccgagctggagctggacctggaccgccccaacatcgaggact
acctgccctccggctcctccatcaacgagcccgccggcaagctgcgcctgcgcgacctgctggacatctcccccatgctgaccga
ggccgccggcgccatcgtggacgactccacacccgctgatcaagtccatcccccccgagccctggaactggaacatctacctgt
tccccctgtggtgatcggcgtgctgatccgctacctgatcctgaccccgcccgcgtgatcgtgctgaccgtgggctggatcaccgtg
atctcctccacatcaccgtgcgcacctgctgaagggccacgactccctgcgcatcaagctggagcgcctgatcgtgcagctgttct
gctcctccacgtggcctcctggaccggcgtggtgaagtaccacggcccccgcccctccatccgcccccagcaggtgtacgtggcc
aaccacacctccatgatcgacttcatcatcctgaaccagatgaccgtgactccgccatcatgcagaagcaccccggctgggtggg
cctgatccagtccaccatcctggagtccgtgggctgcatctggacaaccgcgccgaggccaaggaccgcgagatcgtggccaa
gaagctgctggaccacgtgcacggcgagggcaacaaccccctgctgatcaccccgagggcacctgcgtgaacaaccactactc
cgtgatgttcaagaagggcgccttcgagctgggctgcaccgtgtgccccgtggccatcaagtacaacaagatcttcgtggacgcct
tctggaactccaagaagctgtcatcaccatgcacctgctgcagctgatgacctcctgggccgtggtgtgcgacgtgtggtacttgg
agccccagaccctgaagcccggcgagacccccatcgagttcgccgagcgcgtgcgcgacatcatctccgtgcgcgccggcctg
aagaaggtgccctgggacggctacctgaagtactcccgcccctcccccaagcaccgcgagcgcaagcagcagaccttcgccg
agtccgtgctgcgccgcctggaggagaagggcaacgtggtgcccaccgtgaacTGActcgag
SEQ ID NO: 116 CavigDGAT1
ggtaccATGgccatcgccgacggcggcatcatcggcgccgccggctccatctccgccctgaccgccgacaccgaccccccct
ccctgcgccgccgcaacgtgcccgccggccaggcctccgccgtgtccgccttctccaccgagtccatggccaagcacctgtgcga
cccctcccgcgagccctccccctcccccaagtcctccgacgacggcaaggaccccgacatcggctccgtggactccctgaacga
gaagccctcctcccccgccgccggcaagggccgcctgcagcacgacctgcgatcacctaccgcgcctcctcccccgcccaccg
caaggtgaaggagtcccccctgtcctcctccaacatcttcaagcagtcccacgccggcctgttcaacctgtgcgtggtggtgctggt
ggccgtgaactcccgcctgatcatcgagaacctgatgaagtacggcctgctgatcaagaccggcttctggttctcctcccgctccct
gcgcgactggcccctgttcatgtgctgcctgtccctgcccatcttccccctggccgccttcctggtggagaagctggcccagaagaa
ccgcctgcaggagcccaccgtggtgtgctgccacgtgctgatcacctccgtgtccatcctgtaccccgtgctggtgatcctgcgctg
cgactccgccgtgctgtccggcgtggccctgatgctgttcgcctgcatcgtgtggctgaagctggtgtcctacgcccactccaactac
gacatgcgctacgtggccaagtccctggacaagggcgagcccgtggtggactccgtgatcgccgaccacccctaccgcgtgga
ctacaaggacctggtgtacttcatggtggcccccaccctgtgctaccagctgtcctaccccctgaccccctgcgtgcgcaagtcctg
gatcgcccgccaggtgatgaagctggtgctgttcaccggcgtgatgggcttcatcgtggagcagtacatcaaccccatcgtgcag
aactccaagcaccccctgaagggcgacctgctgtacgccatcgagcgcgtgctgaagctgtccgtgcccaacctgtacgtgtggc
tgtgcatgttctactgcttcttccacctgtggctgaacatcctggccgagctgatctgcttcggcgaccgcgagttctacaaggactgg
tggaacgccaagaccgtggaggagtactggcgcatgtggaacatgcccgtgcacaagtggatggtgcgccacatctacttcccct
gcctgcgcaacggcatcccccgcggcgtggccgtgctgatcgccttcctggtgtccgccgtgttccacgagctgtgcatcgccgtgc
cctgccacgtgttcaagctgtgggccttcatcggcatcatgttccaggtgcccctggtgctggtgtccaactgcctgcagaagaagtt
ccagtcctccatggccggcaacatgttatctggttcatcttctgcatcttcggccagcccatgtgcgtgctgctgtactaccacgacct
gatgaaccgcaagggctcccgcatcgacTGActcgag
SEQ ID NO: 117 ChookDGAT1-1
ggtaccATGgccatcgccgacggcggctccgccggcgccgccggctccatctccggctccgacccctccccctccaccgcccc
ctccctgcgccgccgcaacgcctccgccggccaggccttctccaccgagtccatggcccgcgacctgtgcgacccctcccgcga
gccctccctgtcccccaagtcctccgacgacggcaaggaccccgccgacgacatcggcgccgccgactccgtggactccggcg
gcgtgaaggacgagaagccctcctcccaggccgccgccaaggcccgcctggagcacgacctgcgatcacctaccgcgcctcc
tcccccgcccaccgcaaggtgaaggagtcccccctgtcctcctccaacatcttcaagcagtcccacgccggcctgttcaacctgtg
cgtggtggtgctggtggccgtgaactcccgcctgatcatcgagaacctgatgaagtacggcctgctgatcaagaccggcttctggtt
ctcctcccgctccctgcgcgactggcccctgttcatgtgctgcctgtccctgcccatcaccccctggccgccttcctggtggagaagc
tggcccagaagaaccgcctgcaggagcccaccgtggtgtgctgccacgtgatcatcacctccgtgtccatcctgtaccccgtgctg
gtgatcctgcgctgcgactccgccgtgctgtccggcgtggccctgatgctgttcgcctgcatcgtgtggctgaagctggtgtcctacg
cccacgccaactacgacatgcgctccgtggccaagtccctggacaagggcgagaccgtggccgactccgtgatcgtggaccac
ccctaccgcgtggactacaaggacctggtgtacttcatggtggcccccaccctgtgctaccagctgtcctaccccctgaccccctac
gtgcgcaagtcctgggtggcccgccaggtgatgaagctggtgctgttcaccggcgtgatgggcttcatcgtggagcagtacatcaa
ccccatcgtgcagaactccaagcaccccctgaagggcgacctgctgtacgccatcgagcgcgtgctgaagctgtccgtgcccaa
cctgtacgtgtggctgtgcatgttctactgcttcttccacctgtggctgaacatcctggccgagctgacctgcttcggcgaccgcgagt
tctacaaggactggtggaacgccaagaccgtggaggagtactggcgcatgtggaacatgcccgtgcacaagtggatggtgcgc
cacatctacttcccctgcctgcgcaacggcatcccccgcggcgtggccgtgctgatcgccttcctggtgtccgccgtgttccacgag
ctgtgcatcgccgtgccctgccacgtgttcaagctgtgggccttcatcggcatcatgttccaggtgcccctggtgctggtgtccaactg
cctgcagaagaagttccagtcctccatggccggcaacatgttatctggttcatcttctgcatcttcggccagcccatgtgcgtgctgct
gtactaccacgacctgatgaaccgcaagggctcccgcatcgacTGActcgag
SEQ ID NO: 118 CavigLPCAT
ggtaccATGggcctggtgtccgtggccgccgccatcggcgtgtccgtgcccgtggcccgcttcctgctgtgcttcctggccaccat
ccccgtgtccttcctgtggcgcctggtgcccggccgcctgcccaagcacctgtactccgccgcctccggcgccatcctgtcctacct
gtcatcggcgcctcctccaacctgcacttcatcgtgcccatgaccctgggctacctgtccatgctgttcttccgcccatctccggcct
gctgaccttcttcctgggcttcggctacctgatcggctgccacgtgtactacatgtccggcgacgcctggaaggagggcggcatcg
acgccaccggcgccctgatggtgctgaccctgaaggtgatctcctgctccatgaactacaacgacggcctgctgaaggaggagg
gcctgcgcgagtcccagaagaagaaccgcctgaccaagatgccctccctgatcgagtacttcggctactgcctgtgctgcggctc
ccacttcgccggccccgtgtacgagatgaaggactacctggagtggaccgagggcaagggcatctggtcccgctcccagaagg
agcccaagccctccccatcggcggcgccctgcgcgccatcatccaggccgccgtgtgcatggccatgtacctgtacctggtgccc
caccaccccctgacccgcttcaccgagcccgtgtactacgagtggggatcttccgccgcctgtcctaccagtacatggccgccctg
accgcccgctggaagtactacttcatctggtccatctccgaggcctccctgatcatctccggcctgggcttctccggctggaccgagt
cctccccccccaagccccgctgggaccgcgccaagaacgtggacatcatcggcgtggagttcgccaagtcctccgtgcagctgc
ccctggtgtggaacatccaggtgtccatctggctgcgccactacgtgtacgaccgcctggtgcagaacggcaagcgccccggctt
caccagctgctggccacccagaccgtgtccgccgtgtggcacggcctgtaccccggctacatcatatatcgtgcagtccgccctg
atgatcgccggctcccgcgtgatctaccgctggcagcaggccgtgccccccaagatgggcctggtgaagaacatcttcgtgttctt
caacttcgcctacaccctgctggtgctgaactactccgccgtgggcttcatggtgctgtccatgcacgagaccctggcctcctacgg
ctccgtgtactacatcggcaccatcctgcccatcaccctgatcctgctgtcctacgtgatcaagcccggcaagcccgcccgctccaa
ggcccacaaggagcagTGActcgag
SEQ ID NO: 119 CpalLPCAT
ggtaccATGgagctgggctccgtggccgccgccatcggcgtgtccgtgcccgtggcccgcttcctgctgtgcttcctggccaccat
ccccgtgtccttcctgtggcgcctggtgcccggccgcctgcccaagcacctgtactccgccgcctccggcgccatcctgtcctacct
gtcatcggcccctcctccaacctgcacttcatcgtgcccatgaccctgggctacctgtccatgctgttcttccgcccatctccggcct
gctgaccttcttcctgggcttcggctacctgatcggctgccacgtgtactacatgtccggcgacgcctggaaggagggcggcatcg
acgccaccggcgccctgatggtgctgaccctgaaggtgatctcctgctccatcaactacaacgacggcctgctgaaggaggagg
gcctgcgcgagtcccagaagaagaaccgcctgaccaagatgccctccctgatcgagtacatcggctactgcctgtgctgcggctc
ccacttcgccggccccgtgtacgagatgaaggactacctggagtggaccgagggcaagggcgtgtggtcccactccgagaagg
agcccaagccctccccatcggcggcgccctgcgcgccatcatccaggccgccgtgtgcatggccatgtacatgtacctggtgccc
caccaccccctgtcccgatcaccgagcccgtgtactacgagtggggcacttccgccgcctgtcctaccagtacatggccggcctg
accgcccgctggaagtactacttcatctggtccatctccgaggcctccctgatcatctccggcctgggcttctccggctggaccgagt
cctccccccccaagccccgctgggaccgcgccaagaacgtggacatcatcggcgtggagttcgccaagtcctccgtgcagctgc
ccctggtgtggaacatccaggtgtccacctggctgcgccactacgtgtacgaccgcctggtgcagaacggcaagcgccccggctt
caccagctgctggccacccagaccgtgtccgccatctggcacggcctgtaccccggctacatcatatatcgtgcagtccgccctg
atgatcgccggctcccgcgtgatctaccgctggcagcaggccgtgccccccaagatgggcctggtgaagaacatcttcgtgttctt
caacttcgcctacaccctgctggtgctgaactactccgccgtgggcttcatggtgctgtccatgcacgagaccctggcctcctacgg
ctccgtgtactacatcggcaccatcctgcccatcaccctgatcctgctgtcctacgtgatcaagcccggcaagcccgcccgctccaa
ggcccacaaggagcagTGActcgag
SEQ ID NO: 120 CpauLPCAT
ggtaccATGgagctggagatcggctccgtggccgccgccatcggcgtgtccgtgcccgtggcccgatcctgctgtgatcagg
ccaccatccccgtgtccttcctgtgccgcctgctgcccgcccgcctgcccaagcacctgtactccgccgcctccggcgccatcctgt
cctacctgtcatcggcccctcctccaacctgcacttcatcgtgcccatgtccctgggctacctgtccatgctgttcttccgccccttctcc
ggcctgctgaccttcttcagggatcggctacctgatcggctgccacgtgtactacatgtccggcgacgcctggaaggagggcgg
catcgacgccaccggcgccctgatggtgctgaccctgaaggtgatctcctgctccatcaactacaacgacggcctgctgaaggag
gagggcctgcgcgagtcccagaagaagaaccgcctgaccaagatgccctccctgatcgagtacttcggctactgcctgtgctgcg
gctcccacttcgccggccccgtgtacgagatgaaggactacctggagtggaccgagggcaagggcatctggtcccgctccgaga
aggaccccaagccctccccatcggcggcgccctgcgcgccatcatccaggccgccgtgtgcatggccatgcacatgtacctggt
gccccaccaccccctgacccgcttcaccgagcccgtgtactacgagtggggatcttccgccgcctgtcctaccagtacatggccg
cccagaccgcccgctggaagtactacttcatctggtccatctccgaggcctccctgatcatctccggcctgggcttctccggctggac
cgagtcctccccccccaagccccgctgggacaaggccaagaacgtggacatcatcggcgtggagttcgccaagtcctccgtgca
gctgcccctggtgtggaacatccaggtgtccacctggctgcgccactacgtgtacgaccgcctggtgcagaacggcaagcgccc
cggcttcttccagctgctggccacccagaccgtgtccgccgtgtggcacggcctgtaccccggctacatcatcttcttcgtgcagtcc
gccctgatgatcgccggctcccgcgtgatctaccgctggcagcaggccgtgccccagaagatgggcctggtgaagaacatcttcg
tgttcttcaacttcgcctacaccctgctggtgctgaactactccgccgtgggcttcatggtgctgtccatgcacgagaccctggcctcc
tacggctccgtgtactacatcggcaccatcctgcccatcaccctgatcctgctgtcctacgtgatcaagcccggcaagcccacccg
ctccaaggtgcacaaggagcagTGActcgag
SEQ ID NO: 121 CschuLPCAT
ggtaccATGgagctggagatggagcccctggccgccgccatcggcgtgtccgtggccgtgttccgcttcctggtgtgcttcatcg
ccaccatccccgtgtccttcatctgccgcctggtgcccggcggcctgccccgccacctgttctccgccgcctccggcgccgtgctgtc
ctacctgtcatcggatctcctccaacctgcacttcctggtgcccatgaccctgggctacctgtccatgatcctgttccgccgatctgc
ggcatcctgaccttcttcctgggcttcggctacctgatcggctgccacgtgtactacatgtccggcgacgcctggaaggagggcgg
catcgacgccaccggcgccctgatggtgctgaccctgaaggtgatctcctgctccatcaactacaacgacggcctgctgaaggag
gagggcctgcgcgagtcccagaagaagaaccgcctgatccgcctgccctccctgatcgagtacttcggctactgcctgtgctgcg
gctcccacttcgccggccccgtgtacgagatgaaggactacctggactggaccgagggcaagggcatctggtcccactccgaga
agggccccaagccctcccccctgcgcgccgccctgcgcgccatcatccaggccggcttctgcatggccatgtacctgtacctggtg
ccccactaccccctgacccgcttcaccgaccccgtgtactacgagtggggcatcctgcgccgcctgtcctaccagtacatggcctc
cttcaccgcccgctggaagtactacttcatctggtccatctccgaggcctccctgatcatctccggcctgggcttctccggctggacc
gagtcctccccccccaagccccgctgggaccgcgccaagaacgtggacatcctgggcgtggagctggccaagtcctccgtgca
gatccccctggtgtggaacatccaggtgtccacctggctgcgccactacgtgtacgaccgcctggtgcagaacggcaagcgccc
cggatcctgcagctgctggccacccagaccgtgtccgccatctggcacggcgtgtaccccggctacctgatcacttcgtgcagtcc
gccctgatgatcgccggctcccgcgccatctaccgctggcagcaggccgtgccccccaagatgtccctggtgaagaacaccctg
gtgttcttcaacttcgcctacaccctgctggtgctgaactactccgccgtgggcttcatggtgctgtccatgcacgagaccctggcctc
ctacggctccgtgtactacgtgggcaccatcctgcccgtgaccctgatcctgctgggctacgtgatcaagcccggcaagtcccccc
gctccaaggcctccaaggagcagTGActcgag
SEQ ID NO: 122 CavigPLA2-1
ggtaccATGaacttcgacttcctgtccaacatcccctggttcggcgccaaggcctccgacaacgccggctcctcatcggctccg
ccaccatcgtgatccagcagcccccccccgtgtcccgcggcttcgacatccgccactggggctggccctggtccgtgctgtccgtg
ctgccctggggcaagcccggctgcgacgagctgcgcgccccccccaccaccatcaaccgccgcctgaagcgcaacgccacct
ccatgcactcctccgccgtgcgcggcaacgccgaggccgcccgcgtgcgcttccgcccctacgtgtccaaggtgccctggcaca
ccggcttccgcggcctgctgtcccagctgttcccccgctacggccactactgcggccccaactggtcctccggcaagaacggcgg
ctcccccgtgtgggaccagcgccccatcgactggctggactactgctgctactgccacgacatcggctacgacacccacgacca
ggccaagctgctggaggccgacctggccttcctggagtgcctggagcgcccctcctaccccaccaagggcgacgcccacgtgg
cccacatgtacaagaccatgtgcgtgaccggcctgcgcaacgtgctgatcccctaccgcacccagctgctgcgcctgaactcccg
ccagcccctgatcgacttcggctggctgtccaacgccgcctggaagggctggaacgcccagaagtccTGActcgag
SEQ ID NO: 123 CignPLA2-1
ggtaccATGaacctggacttcctgtccaagatcccctggttcgaggccaaggcctccgagaaccccggcctgaacctgggctcc
accaccatcgtgatcaagcagccccgccagggcttcgacatccgccactggggctggccctggtccgtgctgacctggggcaac
cgcgtgaccgacgaggtgcacgccccccccaccaccatcaaccgccgcctgaagcgcaacgccaccggccccgccgtgcag
ggcgacaccgaggccgcccgcctgcgcttccgcccctacgtgtccaaggtgccctggcacaccggcttccgcggcctgctgtccc
agctgttcccccgctacggccactactgcggccccaactggtcctccggcaagaacggcggctcccccgtgtgggaccagcgcc
ccatcgactggctggactactgctgctactgccacgacatcggctacgacacccacgaccaggccaagctgctggaggccgacc
tggccttcctggagtgcctggagcgcccctcctaccccaccaccggcgacgcccacgtggcccacatgtacaagaccatgtgcgt
gaccggcctgcgcaacgtgctgatcccctaccgcacccagctgctgcgcctgaacttccgccagcccctgatcgacttcggctggc
tgtccaacgccgcctggaagggctggtccgcccagaagaccTGActcgag
SEQ ID NO: 124 CuPSR23PLA2-2
ggtaccATGgtgcacctgccccacaccctgaagctgggcctggtgatcgccatctccatctccggcctgtgcactcctccacccc
cgcccgcgccctgaacgtgggcatccaggccgccggcgtgaccgtgtccgtgggcaagggctgctcccgcaagtgcgagtccg
acactgcaaggtgccccccacctgcgctacggcaagtactgcggcctgatgtactccggctgccccggcgagaagccctgcgac
ggcctggacgcctgctgcatgaagcacgacgcctgcgtgcaggccaagaacaacgactacctgtcccaggagtgctcccagaa
cctgctgaactgcatggcctccaccgcatgtccggcggcaagcagacaagggctccacctgccaggtggacgaggtggtggac
gtgctgaccgtggtgatggaggccgccctgctggccggccgctacctgcacaagcccTGActcgag
SEQ ID NO: 125 CprocPLA2-2
ggtaccATGgtgcacctgccccacaccctgaagctgggcctggtgatcgccatctccatctccggcctgtgcctgtcctccacccc
cgcccgcgccctgaacgtgggcatccaggccgccggcgtgaccgtgtccgtgggcaagggctgctcccgcaagtgcgagtccg
acactgcaaggtgccccccacctgcgctacggcaagtactgcggcctgatgtactccggctgccccggcgagaagccctgcgac
ggcctggacgcctgctgcatgaagcacgacgcctgcgtgcaggccaagaacgacgactacctgtcccaggagtgctcccagaa
cctgctgaactgcatggcctccaccgcatgtccggcggcaagcagacaagggctccacctgccaggtggacgaggtggtggac
gtgctgaccgtggtgatggaggccgccctgctggccggccgctacctgcacaagcccTGActcgag

The constructs containing the codon optimized genes described above driven by the UTEX 1453 SAD2 promoter, were transformed into strain S7858 or S8714. Transformations, cell culture, lipid production and fatty acid analysis were all carried out as described herein. The transgenic strains were selected for their ability to grow on melibiose. Stable transformants were grown under standard lipid production conditions at pH5 (for transgenic strains generated in the strain S7858) or at pH7 (for the transgenic strains generated in the strain S8174) for fatty acid analysis.

### Expression of LPAATs

In WO2013/158938 we disclosed that Cocos nucifera LPAAT enzymes exhibit chain length specificity for the fatty acid acyl-CoA that it attach to the glycerol backbone. We disclosed the impact of expressing CnLPAAT in a transgenic strain also expressing a laurate specific thioesterase. In this example we transformed 5 LPAAT enzymes derived from C8-C10 rich Cuphea species and the CnLPAAT into S7858, and the remaining 8 LPAAT enzymes were transformed into S8174. The resulting fatty acid profiles from a set of representative transgenic lines arising from these transformations are shown in Tables 16 and 17. Expression of these genes as shown in Table 16 resulted in increases in C8:0 and/or- C10:0 fatty acid accumulation.

TABLE 16
Fatty acid profiles of representative transgenic strains of S7858
expressing optimized versions of the CpauLPAAT1, CpalLPAAT1, CignLPAAT1,
CprocLPAAT1, ChookLPAAT1 and CnLPAAT1.

| Sample ID | C8:0 | | | C10:0 | | C12:0 | | C8-C10 | |
|---|---|---|---|---|---|---|---|---|---|
| S6165 | 0.00 | | | 0.00 | | 0.05 | | 0.00 | |
| S7858 | 11.70 | | | 23.36 | | 0.48 | | 35.06 | |
| CpauLPAAT1 @ SAD2-1vD locus | | | | | CprocLPAAT1 @ SAD2-1vD locus | | | | |
| Sample ID | C8:0 | C10:0 | C12:0 | C8-C10 | Sample ID | C8:0 | C10:0 | C12:0 | C8-C10 |
| S7858; D4289-7 | 12.69 | 25.06 | 0.51 | 37.75 | S7858; D4292-15 | 11.86 | 24.05 | 0.46 | 35.91 |
| S7858; D4289-12 | 11.98 | 24.54 | 0.48 | 36.52 | S7858; D4292-11 | 11.49 | 24.01 | 0.48 | 35.50 |
| S7858; D4289-2 | 11.68 | 24.14 | 0.49 | 35.82 | S7858; D4292-22 | 11.49 | 23.81 | 0.47 | 35.30 |
| S7858; D4289-13 | 11.53 | 24.18 | 0.49 | 35.71 | S7858; D4292-3 | 11.46 | 23.76 | 0.46 | 35.22 |
| S7858; D4289-11 | 11.47 | 23.85 | 0.46 | 35.32 | S7858; D4292-24 | 11.38 | 23.64 | 0.46 | 35.02 |
| CpaiLPAAT1 @ SAD2-1vD locus | | | | | ChookLPAAT1 @ SAD2-1vD locus | | | | |
| Sample ID | C8:0 | C10:0 | C12:0 | C8-C10 | Sample ID | C8:0 | C10:0 | C12:0 | C8-C10 |
| S7858; D4290-3 | 13.43 | 25.04 | 0.52 | 38.47 | S7858; D4293-4 | 11.09 | 24.48 | 0.51 | 35.57 |
| S7858; D4290-25 | 12.98 | 24.75 | 0.51 | 37.73 | S7858; D4293-16 | 12.03 | 24.24 | 0.48 | 36.27 |
| S7858; D4290-5 | 12.27 | 25.00 | 0.52 | 37.27 | S7858; D4293-6 | 11.83 | 23.79 | 0.48 | 35.62 |
| S7858; D4290-12 | 11.98 | 24.21 | 0.48 | 36.19 | S7858; D4293-2 | 11.81 | 23.69 | 0.47 | 35.50 |
| S7858; D4290-22 | 11.91 | 23.86 | 0.49 | 35.77 | S7858; D4293-12 | 11.65 | 23.11 | 0.49 | 34.76 |
| CignLPAAT1 @ SAD2-1vD locu | | | | | CnLPAAT1 @ SAD2-1vD locus | | | | |
| Sample ID | C8:0 | C10:0 | C12:0 | C8-C10 | Sample ID | C8:0 | C10:0 | C12:0 | C8-C10 |
| S7858; D4291-13 | 12.95 | 24.78 | 0.52 | 37.73 | S7858; D4404-11 | 12.30 | 24.31 | 0.47 | 36.61 |
| S7858; D4291-20 | 12.13 | 24.63 | 0.49 | 36.76 | S7858; D4404-6 | 12.03 | 24.02 | 0.46 | 36.05 |
| S7858; D4291-15 | 12.12 | 24.35 | 0.47 | 36.47 | S7858; D4404-13 | 11.48 | 23.98 | 0.46 | 35.46 |
| S7858; D4291-22 | 11.94 | 24.50 | 0.47 | 36.44 | S7858; D4404-2 | 11.54 | 23.71 | 0.46 | 35.25 |
| S7858; D4291-7 | 12.11 | 23.14 | 0.50 | 35.25 | S7858; D4404-1 | 11.76 | 23.36 | 0.48 | 35.12 |

TABLE 17
Fatty acid profiles of representative transgenic strains of S8174
expressing CavigLPAAT1, CavigLPAAT2, CpalLPAAT1, CuPSR23LPAAT1,
CkoeLPAAT1, CkoeLPAAT2, CprocLPAAT1 and CprocLPAAT2 before lipase treatment.

| Sample ID | C8:0 | | | C10:0 | | C12:0 | | C8-C10 | |
|---|---|---|---|---|---|---|---|---|---|
| S7485 | 0.00 | | | 0.00 | | 0.07 | | 0.00 | |
| S8174 | 24.32 | | | 9.24 | | 0.37 | | 33.56 | |
| CavigLPAAT1 @ SAD2-1vD locus | | | | | CkoeLPAAT1 @ SAD2-1vD locus | | | | |
| | | | | C8- | | | | | C8- |
| Sample ID | C8:0 | C10:0 | C12:0 | C10 | Sample ID | C8:0 | C10:0 | C12:0 | C10 |
| S8174: D4517-23 | 25.42 | 9.63 | 0.39 | 35.05 | S8174; D4728-8 | 25.44 | 10.31 | 0.46 | 35.75 |
| S8174: D4517-9 | 25.44 | 9.61 | 0.39 | 35.05 | S8174; D4728-10 | 24.15 | 9.51 | 0.43 | 33.66 |
| S8174: D4517-8 | 25.09 | 9.84 | 0.39 | 34.93 | S8174; D4728-5 | 23.88 | 9.56 | 0.45 | 33.44 |
| S8174: D4517-18 | 25.20 | 9.65 | 0.39 | 34.85 | S8174; D4728-6 | 23.58 | 9.28 | 0.40 | 32.86 |
| S8174: D4517-2 | 25.20 | 9.57 | 0.37 | 34.77 | S8174; D4728-9 | 23.47 | 9.25 | 0.40 | 32.72 |
| Cavig LPAAT2 @ SAD2-1vD locus | | | | | CkoeLPAAT2-1 @ SAD2-1vD locus | | | | |
| | | | | C8- | | | | | C8- |
| Sample ID | C8:0 | C10:0 | C12:0 | C10 | Sample ID | C8:0 | C10:0 | C12:0 | C10 |
| S8174: D4518-2 | 24.25 | 9.97 | 0.42 | 34.22 | S8174; D4729-2 | 25.20 | 9.81 | 0.43 | 35.01 |
| S8174: D4518-45 | 24.09 | 9.65 | 0.39 | 33.74 | S8174; D4729-1 | 23.49 | 10.60 | 0.46 | 34.09 |
| S8174: D4518-34 | 23.94 | 9.71 | 0.38 | 33.65 | S8174; D4729-4 | 22.25 | 9.45 | 0.40 | 31.70 |
| S8174: D4518-10 | 24.11 | 9.50 | 0.37 | 33.61 | S8174; D4729-5 | 18.24 | 8.22 | 0.35 | 26.46 |
| S8174: D4518-4 | 23.93 | 9.59 | 0.39 | 33.52 | | | | | |
| CpalLPAAT1 @ SAD2-1vD locus | | | | | CprocLPAAT2 @ SAD2-1vD locus | | | | |
| | | | | C8- | | | | | C8- |
| Sample ID | C8:0 | C10:0 | C12:0 | C10 | Sample ID | C8:0 | C10:0 | C12:0 | C10 |
| S8174: D4519-27 | 25.06 | 9.75 | 0.37 | 34.81 | S8174; D4730-14 | 24.97 | 9.92 | 0.41 | 34.89 |
| S8174: D4519-4 | 23.05 | 10.74 | 0.47 | 33.79 | S8174; D4730-13 | 23.26 | 10.72 | 0.49 | 33.98 |
| S8174: D4519-28 | 24.11 | 9.54 | 0.37 | 33.65 | S8174; D4730-1 | 23.79 | 10.15 | 0.49 | 33.94 |
| S8174: D4519-10 | 23.57 | 9.51 | 0.38 | 33.08 | S8174; D4730-7 | 23.42 | 10.13 | 0.36 | 33.55 |
| S8174: D4519-12 | 23.55 | 9.49 | 0.38 | 33.04 | S8174; D4730-5 | 23.69 | 9.49 | 0.42 | 33.18 |
| CuPSR23LPAAT2-1 @ SAD2-1vD locus | | | | | CuPSR23LPAAT4 @ SAD2-1vD locus | | | | |
| | | | | C8- | | | | | C8- |
| Sample ID | C8:0 | C10:0 | C12:0 | C10 | Sample ID | C8:0 | C10:0 | C12:0 | C10 |
| S8174; D4690-2 | 25.88 | 10.62 | 0.43 | 36.50 | S8174; D4731-1 | 25.94 | 10.87 | 0.56 | 36.81 |
| S8174; D4690-1 | 24.60 | 9.82 | 0.44 | 34.42 | S8174; D4731-3 | 22.79 | 11.52 | 0.59 | 34.31 |
| S8174; D4690-3 | 24.13 | 9.62 | 0.47 | 33.75 | S8174; D4731-5 | 22.89 | 11.22 | 0.53 | 34.11 |
| S8174; D4690-4 | 23.38 | 9.97 | 0.41 | 33.35 | S8174; D4731-2 | 22.99 | 11.07 | 0.45 | 34.06 |
| | | | | | S8174; D4731-4 | 21.15 | 9.63 | 0.43 | 30.78 |

To assess the regiospecific activity of novel LPAAT enzymes, oil extracted from some of these transformants were treated with porcine pancreatic lipase, which selectively hydrolyzes the fatty acids at the sn-1 and sn-3 positions from the glycerol unit of the triacylglycerol, leaving monoacylglycerols (MAGs) with fatty acids located only at the sn-2 position. The resulting mixture of monoacylglycrols (2-MAGs), were isolated by solid phase extraction on an amino propyl cartridge followed by transesterifcation to generate fatty acid methyl esters (FAMEs). The fatty acid profiles of these FAMEs, which represent the profile of fatty acids at the sn-2 position of the various TAGs, were determined by GC-FID. When compared to the fatty acid profiles from transesterification of the oil without lipase treatment, the sn-2 fatty acid profiles show that the expressed LPAAT are selective for the sn-2 position.

The sn-2 analyses after lipase treatment disclosed in Table 18 show that CavigLPAAT1, CpaiLPAAT exhibit selectivity for either C8:0 fatty acids and CpauLPAAT, CignLPAAT are selective for C10:0 fatty acids, demonstrating that the heterologous LPAATs expressed in these transgenic strains have activities that acylate at the sn-2 position with preference for C8:0 or C10:0.

TABLE 18
Fatty acid profiles & sn-2 analysis of representative transgenic strains
of S7858 & S8174 expressing codon optimized versions of the CnLPAAT1,
CpauLPAAT1, CpaiLPAAT1, CignLPAAT1, ChookLPAAT1 and CavigLPAAT1,
CavigLPAAT2, CpalLPAAT1

| | [symbol] | | [symbol] | | [symbol] | | [symbol] | | [symbol] | |
|---|---|---|---|---|---|---|---|---|---|---|
| Fatty Acid | FA profile | sn-2 | FA profile | sn-2 | FA profile | sn-2 | FA profile | sn-2 | FA profile | sn-2 |
| C [symbol] | [symbol] | [symbol] | [symbol] | [symbol] | [symbol] | [symbol] | [symbol] | [symbol] | [symbol] | [symbol] |
| C [symbol] | [symbol] | [symbol] | [symbol] | [symbol] | [symbol] | [symbol] | [symbol] | [symbol] | [symbol] | [symbol] |
| C [symbol] | [symbol] | [symbol] | [symbol] | [symbol] | [symbol] | [symbol] | [symbol] | [symbol] | [symbol] | [symbol] |
| C [symbol] | [symbol] | [symbol] | [symbol] | [symbol] | [symbol] | [symbol] | [symbol] | [symbol] | [symbol] | [symbol] |
| C [symbol] | [symbol] | [symbol] | [symbol] | [symbol] | [symbol] | [symbol] | [symbol] | [symbol] | [symbol] | [symbol] |
| C [symbol] | [symbol] | [symbol] | [symbol] | [symbol] | [symbol] | [symbol] | [symbol] | [symbol] | [symbol] | [symbol] |
| C [symbol] | [symbol] | [symbol] | [symbol] | [symbol] | [symbol] | [symbol] | [symbol] | [symbol] | [symbol] | [symbol] |
| C [symbol] | [symbol] | [symbol] | [symbol] | [symbol] | [symbol] | [symbol] | [symbol] | [symbol] | [symbol] | [symbol] |
| C [symbol] | [symbol] | [symbol] | [symbol] | [symbol] | [symbol] | [symbol] | [symbol] | [symbol] | [symbol] | [symbol] |

| | | | |
|---|---|---|---|
| [symbol] | [symbol] | [symbol] | [symbol] |

| | [symbol] | | [symbol] | | [symbol] | | [symbol] | |
|---|---|---|---|---|---|---|---|---|
| Fatty Acid | FA profile | sn-2 | FA profile | sn-2 | FA profile | sn-2 | FA profile | sn-2 |
| C [symbol] | [symbol] | [symbol] | [symbol] | [symbol] | [symbol] | [symbol] | [symbol] | [symbol] |
| C [symbol] | [symbol] | [symbol] | [symbol] | [symbol] | [symbol] | [symbol] | [symbol] | [symbol] |
| C [symbol] | [symbol] | [symbol] | [symbol] | [symbol] | [symbol] | [symbol] | [symbol] | [symbol] |
| C [symbol] | [symbol] | [symbol] | [symbol] | [symbol] | [symbol] | [symbol] | [symbol] | [symbol] |
| C [symbol] | [symbol] | [symbol] | [symbol] | [symbol] | [symbol] | [symbol] | [symbol] | [symbol] |
| C [symbol] | [symbol] | [symbol] | [symbol] | [symbol] | [symbol] | [symbol] | [symbol] | [symbol] |
| C [symbol] | [symbol] | [symbol] | [symbol] | [symbol] | [symbol] | [symbol] | [symbol] | [symbol] |
| C [symbol] | [symbol] | [symbol] | [symbol] | [symbol] | [symbol] | [symbol] | [symbol] | [symbol] |
| C [symbol] | [symbol] | [symbol] | [symbol] | [symbol] | [symbol] | [symbol] | [symbol] | [symbol] |

| | | |
|---|---|---|
| [symbol] | [symbol] | [symbol] |

[symbol]

indicates data missing or illegible when filed

### Expression of GPATs, DGATs, LPCATs and PLA2s:

The constructs expressing the other acyltransferases (GPAT, DGAT, LPCAT, and PLA2) were transformed into S8174. Stable transformants were grown under standard lipid production conditions at pH7 and analyzed for fatty acid profiles. Similar to the transgenic lines expressing LPAATs, expression of these genes (GPAT, DGAT, LPCAT, and PLA2) also resulted in increases in C8:0-C10:0 fatty acid accumulation (Tables 19a, 19b, and 20). The data presented shows that we have identified novel GPATs, DGATs, LPCATs and PLA2s that show high specificity for C8-C10 fatty acids. To determine the regiospecificity of the novel GPAT, DGAT, LPCAT, and PLA2 enzymes, sn-2 analysis is performed as disclosed in this example and elsewhere herein.

TABLE 19a
Fatty acid profiles of representative transgenic strains of S8174
expressing GPATs and DGATs

| Sample ID | C8:0 | | | C10:0 | | C12:0 | | C8-C10 | |
|---|---|---|---|---|---|---|---|---|---|
| S7485 | 0.00 | | | 0.00 | | 0.07 | | 0.00 | |
| S8174 | 24.61 | | | 9.10 | | 0.42 | | 33.71 | |
| CavigGPAT9 @ SAD2-1vD locus | | | | | CignGPAT9-2 @ SAD2-1vD locus | | | | |
| Sample ID | C8:0 | C10:0 | C12:0 | C8-C10 | Sample ID | C8:0 | C10:0 | C12:0 | C8-C10 |
| S8174; D4551-8 | 24.52 | 9.05 | 0.36 | 33.57 | S8174; D4554-9 | 24.49 | 9.13 | 0.45 | 33.62 |
| S8174; D4551-7 | 24.24 | 9.04 | 0.36 | 33.28 | S8174; D4554-3 | 24.28 | 8.90 | 0.42 | 33.18 |
| S8174; D4551-2 | 23.93 | 8.92 | 0.37 | 32.85 | S8174; D4554-7 | 23.86 | 8.96 | 0.43 | 32.82 |
| S8174; D4551-6 | 23.63 | 8.92 | 0.41 | 32.55 | S8174; D4554-8 | 23.99 | 8.81 | 0.39 | 32.80 |
| S8174; D4551-3 | 23.35 | 8.90 | 0.43 | 32.25 | S8174; D4554-4 | 23.87 | 8.78 | 0.4 | 32.65 |
| ChookGPAT9-1 @ SAD2-1vD locus | | | | | CpalGPAT9-1 @ SAD2-1vD locus | | | | |
| Sample ID | C8:0 | C10:0 | C12:0 | C8-C10 | Sample ID | C8:0 | C10:0 | C12:0 | C8-C10 |
| S8174; D4552-6 | 23.57 | 9.00 | 0.36 | 32.57 | S8174; D4724-6 | 25.61 | 9.52 | 0.39 | 35.13 |
| S8174; D4552-4 | 23.62 | 8.87 | 0.37 | 32.49 | S8174; D4724-7 | 24.91 | 9.36 | 0.41 | 34.27 |
| S8174; D4552-9 | 23.39 | 8.97 | 0.40 | 32.36 | S8174; D4724-2 | 24.43 | 9.46 | 0.39 | 33.89 |
| S8174; D4552-8 | 23.28 | 8.80 | 0.40 | 32.08 | S8174; D4724-5 | 24.01 | 9.25 | 0.39 | 33.26 |
| S8174; D4552-11 | 23.18 | 8.80 | 0.44 | 31.98 | S8174; D4724-4 | 24.30 | 8.93 | 0.39 | 33.23 |
| CignGPAT9-1 @ SAD2-1vD locus | | | | | CpalGPAT9-2 @ SAD2-1vD locus | | | | |
| Sample ID | C8:0 | C10:0 | C12:0 | C8-C10 | Sample ID | C8:0 | C10:0 | C12:0 | C8-C10 |
| S8174; D4553-12 | 25.19 | 9.42 | 0.40 | 34.61 | S8174; D4725-5 | 24.24 | 10.30 | 0.48 | 34.54 |
| S8174; D4685-1 | 24.33 | 10.24 | 0.46 | 34.57 | S8174; D4725-6 | 24.81 | 9.29 | 0.41 | 34.10 |
| S8174; D4553-15 | 25.11 | 9.33 | 0.41 | 34.44 | S8174; D4725-7 | 24.35 | 9.51 | 0.42 | 33.86 |
| S8174; D4553-1 | 24.56 | 9.50 | 0.44 | 34.06 | S8174; D4725-8 | 24.37 | 9.39 | 0.40 | 33.76 |
| S8174; D4553-6 | 24.74 | 9.16 | 0.40 | 33.90 | S8174; D4725-9 | 24.28 | 9.29 | 0.41 | 33.57 |

TABLE 19b
Fatty acid profiles of representative transgenic strains of S8174
expressing DGATs

| Sample ID | C8:0 | C10:0 | C12:0 | C8-C10 |
|---|---|---|---|---|
| S7485 | 0.00 | 0.00 | 0.07 | 0.00 |
| S8174 | 24.61 | 9.10 | 0.42 | 33.71 |

Cavig DGAT1 @ SAD2-1vD locus

| | | | | |
|---|---|---|---|---|
| S8174; D4549-7 | 24.89 | 9.28 | 0.36 | 34.17 |
| S8174; D4549-6 | 24.53 | 9.04 | 0.47 | 33.57 |
| S8174; D4549-4 | 23.93 | 8.99 | 0.41 | 32.92 |
| S8174; D4549-1 | 23.93 | 8.97 | 0.38 | 32.90 |
| S8174; D4549-3 | 23.76 | 8.9 | 0.36 | 32.66 |

Chook DGAT1 @ SAD2-1vD locus

| | | | | |
|---|---|---|---|---|
| S8174; D4550-1 | 24.67 | 9.12 | 0.41 | 33.79 |
| S8174; D4550-3 | 24.64 | 9.06 | 0.42 | 33.70 |
| S8174; D4682-1 | 23.72 | 9.68 | 0.5 | 33.40 |
| S8174; D4682-2 | 23.49 | 9.66 | 0.41 | 33.15 |
| S8174; D4550-2 | 22.42 | 8.81 | 0.41 | 31.23 |

TABLE 20
Fatty acid profiles of representative transgenic strains of S8174
expressing LPCATs and PLA2s

| Sample ID | C8:0 | | | C10:0 | | C12:0 | | C8-C10 | |
|---|---|---|---|---|---|---|---|---|---|
| S7485 | 0.00 | | | 0.00 | | 0.07 | | 0.00 | |
| S8174 | 24.61 | | | 9.10 | | 0.42 | | 33.71 | |
| Cavig LPCAT @ SAD2-1vD locus | | | | | Cavig PLA2-1 @ SAD2-1vD locus | | | | |
| Sample ID | C8:0 | C10:0 | C12:0 | C8-C10 | Sample ID | C8:0 | C10:0 | C12:0 | C8-C10 |
| S8174; D4555-1 | 26.6 | 9.38 | 0.47 | 35.98 | S8174; D4732-1 | 26.31 | 11.24 | 0.60 | 37.55 |
| S8174; D4555-3 | 26.4 | 9.47 | 0.39 | 35.87 | S8174; D4732-2 | 25.30 | 11.88 | 0.50 | 37.18 |
| S8174; D4688-1 | 25.95 | 9.67 | 0.44 | 35.62 | S8174; D4732-3 | 25.29 | 11.01 | 0.48 | 36.30 |
| S8174; D4688-3 | 25.47 | 9.89 | 0.44 | 35.36 | S8174; D4732-4 | 25.30 | 11.00 | 0.47 | 36.30 |
| S8174; D4555-2 | 25.52 | 9.55 | 0.36 | 35.07 | S8174; D4732-5 | 25.07 | 11.20 | 0.44 | 36.27 |
| Cpau LPCAT @ SAD2-1vD locus | | | | | CignPLA2-1 @ SAD2-1vD locus | | | | |
| Sample ID | C8:0 | C10:0 | C12:0 | C8-C10 | Sample ID | C8:0 | C10:0 | C12:0 | C8-C10 |
| S8174; D4556-3 | 25.55 | 9.21 | 0.43 | 34.76 | S8174; D4734-6 | 26.39 | 11.34 | 0.47 | 37.73 |
| S8174; D4556-4 | 25.24 | 9.46 | 0.41 | 34.70 | S8174; D4734-1 | 26.17 | 10.90 | 0.46 | 37.07 |
| S8174; D4689-7 | 24.63 | 9.86 | 0.43 | 34.49 | S8174; D4734-5 | 25.58 | 11.12 | 0.57 | 36.70 |
| S8174; D4556-1 | 25.18 | 9.13 | 0.42 | 34.31 | S8174; D4734-4 | 25.48 | 11.17 | 0.57 | 36.65 |
| S8174; D4689-6 | 24.05 | 9.89 | 0.48 | 33.94 | S8174; D4734-2 | 24.75 | 11.32 | 0.46 | 36.07 |
| Cpal LPCAT @ SAD2-1vD locus | | | | | CuPSR23PLA2-2 @ SAD2-1vD locus | | | | |
| Sample ID | C8:0 | C10:0 | C12:0 | C8-C10 | Sample ID | C8:0 | C10:0 | C12:0 | C8-C10 |
| S8174; D4726-4 | 26.34 | 9.76 | 0.41 | 36.10 | S8174; D4735-5 | 25.81 | 11.16 | 0.44 | 36.97 |
| S8174; D4726-2 | 25.92 | 9.9 | 0.44 | 35.82 | S8174; D4735-1 | 25.95 | 10.92 | 0.47 | 36.87 |
| S8174; D4726-3 | 26.15 | 9.62 | 0.41 | 35.77 | S8174; D4735-8 | 25.54 | 10.91 | 0.42 | 36.45 |
| S8174; D4726-5 | 26.09 | 9.55 | 0.41 | 35.64 | S8174; D4735-7 | 25.45 | 10.95 | 0.44 | 36.40 |
| S8174; D4726-1 | 25.64 | 9.57 | 0.39 | 35.21 | S8174; D4735-6 | 25.51 | 10.88 | 0.41 | 36.39 |
| Cschu LPCAT @ SAD2-1vD locus | | | | | Cproc PLA2-2 @ SAD2-1vD locus | | | | |
| Sample ID | C8:0 | C10:0 | C12:0 | C8-C10 | Sample ID | C8:0 | C10:0 | C12:0 | C8-C10 |
| S8174; D4727-1 | 26.24 | 9.95 | 0.45 | 36.19 | S8174; D4736-2 | 25.60 | 10.87 | 0.42 | 36.47 |
| S8174; D4727-7 | 26.26 | 9.84 | 0.42 | 36.10 | S8174; D4736-4 | 25.55 | 10.76 | 0.40 | 36.31 |
| S8174; D4727-9 | 26.13 | 9.87 | 0.42 | 36.00 | S8174; D4736-3 | 25.40 | 10.87 | 0.36 | 36.27 |
| S8174; D4727-11 | 25.99 | 9.97 | 0.44 | 35.96 | S8174; D4736-5 | 25.45 | 10.46 | 0.39 | 35.91 |
| S8174; D4727-16 | 26.28 | 9.68 | 0.44 | 35.96 | S8174; D4736-1 | 24.34 | 11.06 | 0.48 | 35.40 |

### Example 7: Expression of LPAAT and/or DGAT in Prototheca to Produce High SOS and Low Trisaturated TAGs

In this example we describe genetically engineered Prototheca moriformis strains in which we have modified fatty acid and triacylglycerol biosynthesis to maximize the accumulation of Stearoyl-Oleoyl-Stearoyl (SOS) TAGs, and minimize the production of trisaturated TAGs. Tailored oils from these strains resemble plant seed oils known as “structuring fats”, which have high proportions of Saturated-Oleate-Saturated TAGs and low levels of trisaturates. These structuring fats (often called “butters”) are generally solid at room temperature but melt sharply between 35-40° C.

High-SOS strains were obtained by three successive transformations beginning with strain S5100, a classically improved derivative, of a wild type isolate of Prototheca moriformis, S376. Strain S5100 was transformed with plasmid pSZ5654 to generate strain S8754, which produces an oil with increased stearic acid (C18:0) content, lower palmitic acid (C16:0) and reduced linoleic acid (C18:2cisΔ9,12) content relative to S5100. In turn, strain S8754 was transformed with plasmid pSZ5868 to generate strain S8813, which produces oil with higher C18:0, lower C16:0 and improved sn-2 selectivity compared to S8754. Finally, strain S8813 was transformed with plasmids pSZ6383 or pSZ6384 to generate strains S9119, S9120 and S9121, producing oils rich in C18:0 with reduced levels of C18:2cisΔ9,12 and improved sn-3 selectivity.

Construct Used for SAD2 Knockout in S5100

The first intermediate strains were prepared by transformation of strain S5100 with integrative plasmid pSZ5654 (SAD2-1vD::PmKASII-1tp_PmKASII-1_FLAG-CvNR:CrTUB2-PmFAD2hpA-CvNR:PmHXT1-2v2-ScarMEL1-PmPGK::SAD2-1vE). The construct targeted ablation of allele 1 of the endogenous stearoyl-ACP desaturase 2 gene (SAD2), concomitant with expression of the PmKASII gene encoding P. moriformis β-keto-acyl-ACP synthase, and a RNAi hairpin sequence to down-regulate fatty acid desaturase (FAD2) gene expression. Deletion of one allele of SAD2 reduced SAD activity, resulting in elevated levels of C18:0. Overexpression of PmKASII stimulated elongation of C16:0 to C18:0, further increasing C18:0. FAD2 is responsible for the conversion of C18:1cisΔ9 (oleic) to C18:2cisΔ9,12 (linoleic) fatty acids, and RNAi of FAD2 resulted in decreased C18:2. Thus, the first intermediate strains had higher levels of C18:0 and decreased C16:0 and C18:2 fatty acid levels relative to the S5100 parent. The Saccharomyces carlsbergensis MEL1 gene, encoding a secreted melibiase served as a selectable marker as part of plasmid pSZ5654, enabling the strain to grow on melibiose.

The sequence of the pSZ5654 transforming DNA is provided below. Relevant restriction sites in the construct are indicated in lowercase, bold and underlining and are 5′-3′ PmeI, SpeI, AscI, ClaI, SacI, AvrII, EcoRV, EcoRI, SpeI, BsiWI, XhoI, SacI, KpnI, SnaBI, BspQI and PmeI, respectively. PmeI sites delimit the 5′ and 3′ ends of the transforming DNA. Bold, lowercase sequences represent SAD2-1 5′ genomic DNA that permit targeted integration at the SAD2-1 locus via homologous recombination. Proceeding in the 5′ to 3′ direction, bold, lowercase sequences represent SAD2-1 5′ genomic DNA sequences that permit targeted integration at the FATA-1 locus via homologous recombination. The initiator ATG of the sequence encoding the P. moriformis KASII-1 transit peptide (PmKASII-1tp) is indicated by uppercase, bold italics, and the PmKASII-1tp sequence located between the ATG and the AscI site is indicated with lowercase, underlined italics. The PmKASII-1 coding region is indicated by lowercase italics. A sequence encoding a 3×FLAG tag fused to the C-terminus of PmKASII-1 is represented by uppercase italics, and the TGA terminator codon is indicated with uppercase, bold italics. The Chlorella vulgaris nitrate reductase (NR) gene 3′ UTR is indicated by lowercase underlined text. A spacer sequence is represented by lowercase text. The C. reinhardtii TUB2 promoter, driving expression of the PmFAD2hpA sequence is indicated by boxed text. Bold italics denote the PmFAD2hpA sequence followed by lowercase underlined text representing C. vulgaris nitrate reductase 3′ UTR. A second spacer sequence is represented by lowercase text. The P. moriformis HXT1 promoter driving the expression of the S. carlbergensis MEL1 gene is indicated by boxed text. The initiator ATG and terminator TGA for MEL1 gene are indicated by uppercase, bold italics while the coding region is indicated in lowercase italics. The P. moriformis PGK 3′ UTR is indicated by lowercase underlined text. The SAD2-1 3′ genomic region indicated by bold, lowercase text.

Nucleotide sequence of transforming DNA contained in pSZ5654
SEQ ID NO: 126
gtttaaacgccggtcaccacccgcatgctcgtactacagcgcacgcaccgcttcgtgatccaccgggtgaacgtagtcctcgacgg
aaacatctggttcgggcctcctgcttgcactcccgcccatgccgacaacctttctgctgttaccacgacccacaatgcaacgcgaca
cgaccgtgtgggactgatcggttcactgcacctgcatgcaattgtcacaagcgcttactccaattgtattcgtttgttttctgggagc
agttgctcgaccgcccgcgtcccgcaggcagcgatgacgtgtgcgtggcctgggtgtttcgtcgaaaggccagcaaccctaaatcg
caggcgatccggagattgggatctgatccgagtttggaccagatccgccccgatgcggcacgggaactgcatcgactcggcgcgg
aacccagctttcgtaaatgccagattggtgtccgatacctggatttgccatcagcgaaacaagacttcagcagcgagcgtatttgg
cgggcgtgctaccagggttgcatacattgcccatttctgtctggaccgctttactggcgcagagggtgagttgatggggttggcagg
catcgaaacgcgcgtgcatggtgtgcgtgtctgttttcggctgcacgaattcaatagtcggatgggcgacggtagaattgggtgtg
gcgctcgcgtgcatgcctcgccccgtcgggtgtcatgaccgggactggaatcccccctcgcgaccatcttgctaacgctcccgactc

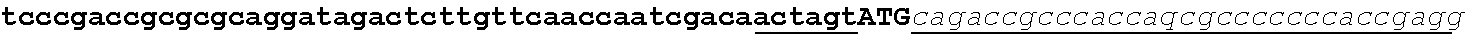

gccactacttcaacacccacctacccaccacctcccaccacaccatacaccacacctaatcccacatcacccacgggcgcgccgc
cgccgccgccgacgccaaccccgcccgccccgagcgccgcgtggtgatcaccggccagggcgtggtgacctccctgggccag
accatcgagcagttctactcctccctgctggagggcgtgtccggcatctcccagatccagaagttcgacaccaccggctacacc
accaccatcgccggcgagatcaagtccctgcagctggacccctacgtgcccaagcgctgggccaagcgcgtggacgacgtga
tcaagtacgtgtacatcgccggcaagcaggccctggagtccgccggcctgcccatcgaggccgccggcctggccggcgccgg
cctggaccccgccctgtgcggcgtgctgatcggcaccgccatggccggcatgacctccttcgccgccggcgtggaggccctgac
ccgcggcggcgtgcgcaagatgaaccccttctgcatccccttctccatctccaacatgggcggcgccatgctggccatggacatc
ggcttcatgggccccaactactccatctccaccgcctgcgccaccggcaactactgcatcctgggcgccgccgaccacatccgcc
gcggcgacgccaacgtgatgctggccggcggcgccgacgccgccatcatcccctccggcatcggcggcttcatcgcctgcaag
gccctgtccaagcgcaacgacgagcccgagcgcgcctcccgcccctgggacgccgaccgcgacggcttcgtgatgggcgagg
gcgccggcgtgctggtgctggaggagctggagcacgccaagcgccgcggcgccaccatcctggccgagctggtgggcggcg
ccgccacctccgacgcccaccacatgaccgagcccgacccccagggccgcggcgtgcgcctgtgcctggagcgcgccctggag
cgcgcccgcctggcccccgagcgcgtgggctacgtgaacgcccacggcacctccacccccgccggcgacgtggccgagtaccg
cgccatccgcgccgtgatcccccaggactccctgcgcatcaactccaccaagtccatgatcggccacctgctgggcggcgccgg
cgccgtggaggccgtggccgccatccaggccctgcgcaccggctggctgcaccccaacctgaacctggagaaccccgcccccg
gcgtggaccccgtggtgctggtgggcccccgcaaggagcgcgccgaggacctggacgtggtgctgtccaactccttcggcttc
ggcggccacaactcctgcgtgatcttccgcaagtacgacgagATGGACTACAAGGACCACGACGGCGACTACAA

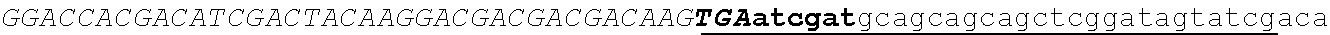

cactctggacgctggtcgtgtgatggactgttgccgccacacttgctgccttgacctgtgaatatccctgccgcttttatcaaacagcc
tcagtgtgtttgatcttgtgtgtacgcgcttttgcgagttgctagctgcttgtgctatttgcgaataccacccccagcatccccttccctc
gtttcatatcgcttgcatcccaaccgcaacttatctacgctgtcctgctatccctcagcgctgctcctgctcctgctcactgcccctcgca
cagccttggtttgggctccgcctgtattctcctggtactgcaacctgtaaaccagcactgcaatgctgatgcacgggaagtagtggga
tgggaacacaaatggagagctccgcgtctcgaacagagcgcgcagaggaacgctgaaggtctcgcctctgtcgcacctcagcgcg
gcatacaccacaataaccacctgacgaatgcgcttggttcttcgtccattagcgaagcgtccggttcacacacgtgccacgttggcg

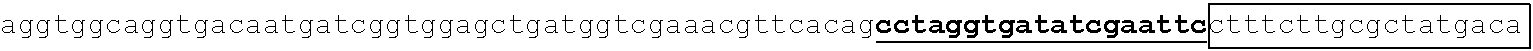

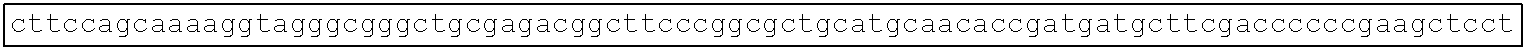

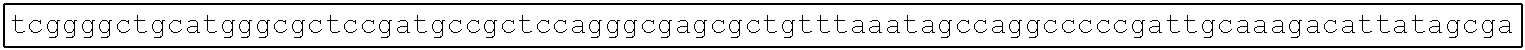

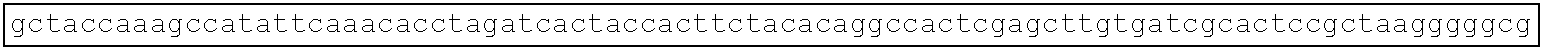

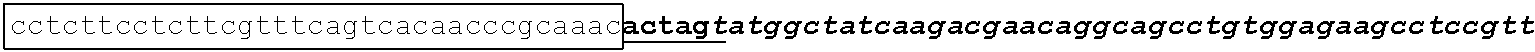

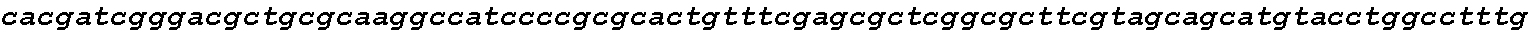

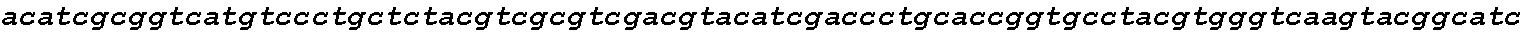

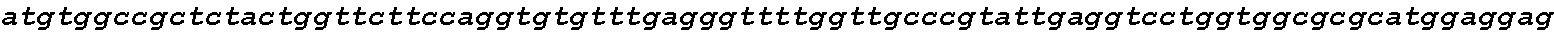

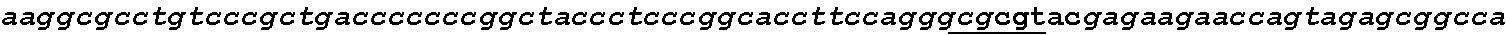

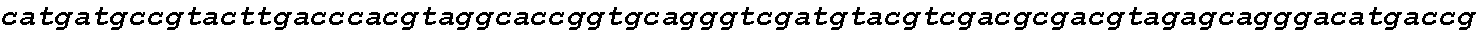

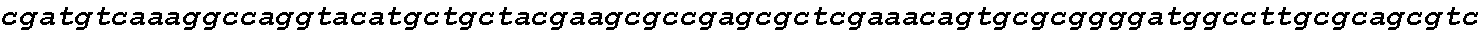

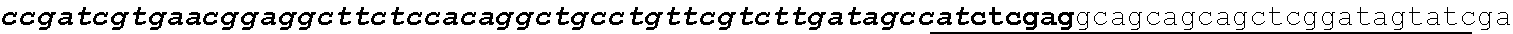

cacactctggacgctggtcgtgtgatggactgttgccgccacacttgctgccttgacctgtgaatatccctgccgcttttatcaaacag
cctcagtgtgtttgatcttgtgtgtacgcgcttttgcgagttgctagctgcttgtgctatttgcgaataccacccccagcatccccttccc
tcgtttcatatcgcttgcatcccaaccgcaacttatctacgctgtcctgctatccctcagcgctgctcctgctcctgctcactgcccctcg
cacagccttggtttgggctccgcctgtattctcctggtactgcaacctgtaaaccagcactgcaatgctgatgcacgggaagtagtgg
gatgggaacacaaatggaaagctgtagagctcgatctaagtaagattcgaagcgctcgaccgtgccggacggactgcagccccat
gtcgtagtgaccgccaatgtaagtgggctggcgtttccctgtacgtgagtcaacgtcactgcacgcgcaccaccctctcgaccggca

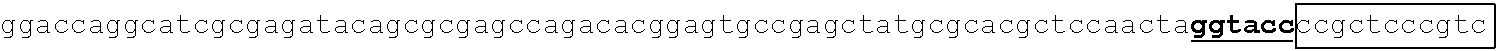

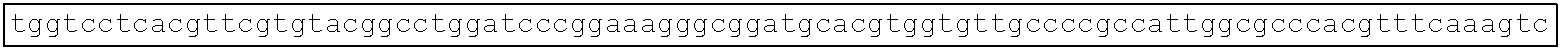

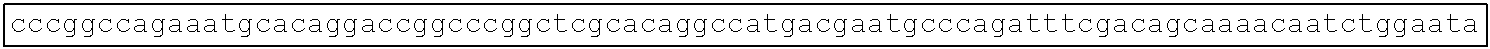

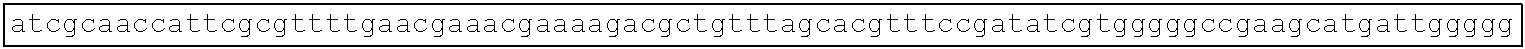

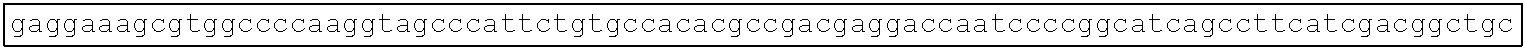

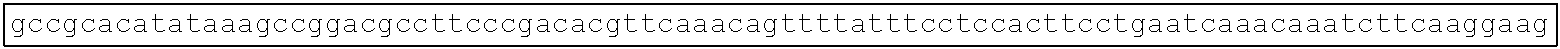

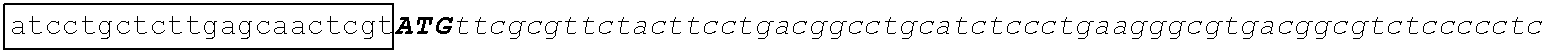

ctacaacggcctgggcctgacgccccagatgggctgggacaactggaacacgttcgcctgcgacgtctccgagcagctgctgc
tggacacggccgaccgcatctccgacctgggcctgaaggacatgggctacaagtacatcatcctggacgactgctggtcctcc
ggccgcgactccgacggcttcctggtcgccgacgagcagaagttccccaacggcatgggccacgtcgccgaccacctgcaca
acaactccttcctgttcggcatgtactcctccgcgggcgagtacacgtgcgccggctaccccggctccctgggccgcgaggagg
aggacgcccagttcttcgcgaacaaccgcgtggactacctgaagtacgacaactgctacaacaagggccagttcggcacgcc
cgagatctcctaccaccgctacaaggccatgtccgacgccctgaacaagacgggccgccccatcttctactccctgtgcaactg
gggccaggacctgaccttctactggggctccggcatcgcgaactcctggcgcatgtccggcgacgtcacggcggagttcacgc
gccccgactcccgctgcccctgcgacggcgacgagtacgactgcaagtacgccggcttccactgctccatcatgaacatcctga
acaaggccgcccccatgggccagaacgcgggcgtcggcggctggaacgacctggacaacctggaggtcggcgtcggcaac
ctgacggacgacgaggagaaggcgcacttctccatgtgggccatggtgaagtcccccctgatcatcggcgcgaacgtgaaca
acctgaaggcctcctcctactccatctactcccaggcgtccgtcatcgccatcaaccaggactccaacggcatccccgccacgcg
cgtctggcgctactacgtgtccgacacggacgagtacggccagggcgagatccagatgtggtccggccccctggacaacggc
gaccaggtcgtggcgctgctgaacggcggctccgtgtcccgccccatgaacacgaccctggaggagatcttcttcgactccaac
ctgggctccaagaagctgacctccacctgggacatctacgacctgtgggcgaaccgcgtcgacaactccacggcgtccgccat
cctgggccgcaacaagaccgccaccggcatcctgtacaacgccaccgagcagtcctacaaggacggcctgtccaagaacgac
acccgcctgttcggccagaagatcggctccctgtcccccaacgcgatcctgaacacgaccgtccccgcccacggcatcgcgttct

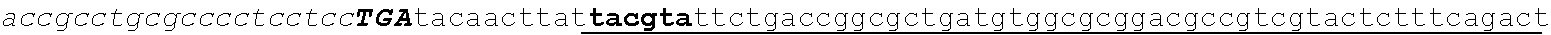

ttactcttgaggaattgaacctttctcgcttgctggcatgtaaacattggcgcaattaattgtgtgatgaagaaagggtggcacaaga
tggatcgcgaatgtacgagatcgacaacgatggtgattgttatgaggggccaaacctggctcaatcttgtcgcatgtccggcgcaat
gtgatccagcggcgtgactctcgcaacctggtagtgtgtgcgcaccgggtcgctttgattaaaactgatcgcattgccatcccgtcaa
ctcacaagcctactctagctcccattgcgcactcgggcgcccggctcgatcaatgttctgagcggagggcgaagcgtcaggaaatcg
tctcggcagctggaagcgcatggaatgcggagcggagatcgaatcaggatccttagggagcgacgagtgtgcgtgcggggctggc
gggagtgggacgccctcctcgctcctctctgttctgaacggaacaatcggccaccccgcgctacgcgccacgcatcgagcaacga
agaaaaccccccgatgataggttgcggtggctgccgggatatagatccggccgcacatcaaagggcccctccgccagagaagaa
gctcctttcccagcagactccttctgctgccaaaacacttctctgtccacagcaacaccaaaggatgaacagatcaacttgcgtctc
cgcgtagcttcctcggctagcgtgcttgcaacaggtccctgcactattatcttcctgctttcctctgaattatgcggcaggcgagcgct
cgctctggcgagcgctccttcgcgccgccctcgctgatcgagtgtacagtcaatgaatggtcctgggcgaagaacgagggaatttg
tgggtaaaacaagcatcgtctctcaggccccggcgcagtggccgttaaagtccaagaccgtgaccaggcagcgcagcgcgtccgt
gtgcgggccctgcctggcggctcggcgtgccaggctcgagagcagctccctcaggtcgccttggacggcctctgcgaggccggtga
gggcctgcaggagcgcctcgagcgtggcagtggcggtcgtatccgggtcgccggtcaccgcctgcgactcgccatccgaagagcg
tttaaac

Construct pSZ5654 was transformed into S5100. Primary transformants were clonally purified and screened under standard lipid production conditions at pH 5. Integration of pSZ5654 at the SAD2-1 locus was verified by DNA blot analysis. The fatty acid profiles and lipid titers of lead strains were assayed in 50-mL shake flasks (Table 21). S8754 was selected as the lead strain for additional rounds of genetic engineering. As shown in Table 21, C16:0 decreased from 17.6% to less than 6%, C18:0 increased from 4.3% to about 28%, C18:2 decreased from 5.8% to 1.3%.

TABLE 21
Fatty acid profiles of SAD2-1 ablation strains.

| Sample ID | S5100 | S8741 | S8742 | S8743 | S8744 | S8745 | S8746 | S8752 | S8753 | S8754 |
|---|---|---|---|---|---|---|---|---|---|---|
| C14:0 | 0.7 | 0.6 | 0.6 | 0.6 | 0.6 | 0.6 | 0.6 | 0.6 | 0.6 | 0.6 |
| C16:0 | 17.6 | 5.9 | 5.9 | 5.8 | 5.9 | 5.9 | 5.9 | 5.9 | 5.8 | 5.9 |
| C16:1 cis-9 | 0.4 | 0.0 | 0.0 | 0.0 | 0.0 | 0.0 | 0.0 | 0.0 | 0.0 | 0.1 |
| C18:0 | 4.3 | 28.2 | 28.1 | 27.7 | 27.8 | 27.4 | 28.2 | 28.3 | 28.3 | 28.1 |
| C18:1 | 69.8 | 60.1 | 60.2 | 60.6 | 60.5 | 60.9 | 60.0 | 60.0 | 60.0 | 60.0 |
| C18:2 | 5.8 | 1.3 | 1.3 | 1.3 | 1.3 | 1.3 | 1.3 | 1.3 | 1.2 | 1.3 |
| C18:3 α | 0.5 | 0.3 | 0.3 | 0.3 | 0.3 | 0.3 | 0.3 | 0.3 | 0.3 | 0.3 |
| C20:0 | 0.3 | 2.2 | 2.2 | 2.2 | 2.2 | 2.2 | 2.2 | 2.2 | 2.2 | 2.2 |
| saturates | 23.2 | 37.5 | 37.5 | 37.1 | 37.2 | 36.8 | 37.7 | 37.7 | 37.7 | 37.6 |
| lipid (g/L) | 13.5 | 12.8 | 12.5 | 12.5 | 12.5 | 12.3 | 12.3 | 12.3 | 12.4 | 12.3 |

### Construct Used for FATA-1 Knockout in S8754

The second intermediate strains were prepared by transformation of strain S8754 with integrative plasmid pSZ5868 (FATA-1vB::CpSAD1tp_GarmFATA1(G108A)_FLAG-PmSAD2-1:PmG3PDH-1-TcLPAT2-PmATP:CrTUB2-ScSUC2-PmPGH::FATA-1vC). This construct targeted ablation of allele 1 of the endogenous fatty acyl-ACP thioesterase gene (FATA-1), and contained expression modules for GarmFATA1(G108A), encoding a variant of the Garcinia mangostana FATA1 thioesterase with improved activity, and TcLPAT2 encoding the Theobroma cacao lysophosphatidic acid acyltransferase (LPAAT). Deletion of one copy of FATA-1 reduced endogenous thioesterase activity, further reducing C16:0 accumulation. Expression of GarmFATA1(G108A) stimulated C18:0-ACP hydrolysis, further increasing C18:0. TcLPAT2 had superior specificity for transfer of C18:1 to the sn-2 position of triacylglycerides than the endogeneous LPAAT, leading to reduced accumulation of trisaturates. The second intermediate strains had increased C18:0 and lower C16:0 compared their parent, S8754. The S. cerevisiae SUC2 gene encoding a secreted sucrose invertase, served as a selectable marker as part of plasmid pSZ5868 and enabled the strain to grow on sucrose.

The sequence of the pSZ5868 transforming DNA is provided below. Relevant restriction sites in the construct are indicated in lowercase, bold and underlining and are 5′-3′ BspQI, PmeI, SpeI, AscI, ClaI, SacI, AvrII, NdeI, NsiI, AfIII, KpnI, XbaI, MfeI, BamHI, BspQI and PmeI, respectively. BspQI and PmeI sites delimit the 5′ and 3′ ends of the transforming DNA. Proceeding in the 5′ to 3′ direction, bold, lowercase sequences represent FATA-1 5′ genomic DNA that permit targeted integration at the FATA-1 locus via homologous recombination. The initiator ATG of the sequence encoding the C. protothecoides SAD1 transit peptide (CpSAD1tp) is indicated by uppercase, bold italics, and the remainder of the CpSAD1tp sequence located between the ATG and the AscI site is indicated with lowercase, underlined italics. The GarmFATA1(G108A) coding region is indicated by lowercase italics. A sequence encoding a 3×FLAG tag fused to the C-terminus of GarmFATA1(G108A) is represented by uppercase italics, and the TGA terminator codon is indicated with uppercase, bold italics. The P. moriformis SAD2-1 3′ UTR is indicated by lowercase underlined text. A spacer sequence is represented by lowercase text. The P. moriformis G3PDH-1 promoter, driving expression of the TcLPAT2 sequence is indicated by boxed text. The initiator ATG and terminator TGA codons of the TcLPAT2 gene are indicated by uppercase, bold italics, while the remainder of the coding region is represented with italics. Lowercase underlined text represents the P. moriformis ATP 3′ UTR. A second spacer sequence is represented by lowercase text. The C. reinhardtii TUB2 promoter driving the expression of the S. cerevisiae SUC2 gene is indicated by boxed text. The initiator ATG and terminator TGA for SUC2 are indicated by uppercase, bold italics while the coding region is indicated in lowercase italics. The P. moriformis PGH 3′ UTR is indicated by lowercase underlined text. The FATA-1 3′ genomic region indicated by bold, lowercase text.

Nucleotide sequence of transforming DNA contained in pSZ5868
SEQ ID NO: 127
gaagagcgcccaatgtttaaacctcttttgctgcgtctcctcaggcttgggggcctccttgggcttgggtgccgccatgatctgcgcg
catcagagaaacgttgctggtaaaaaggagcgcccggctgcgcaatatatatataggcatgccaacacagcccaacctcactcg
ggagcccgtcccaccacccccaagtcgcgtgccttgacggcatactgctgcagaagcttcatgagaatgatgccgaacaagaggg
gcacgaggacccaatcccggacatccttgtcgataatgatctcgtgagtccccatcgtccgcccgacgctccggggagcccgccga
tgctcaagacgagagggccctcgaccaggaggggctggcccgggcgggcactggcgtcgaaggtgcgcccgtcgttcgcctgca
gtcctatgccacaaaacaagtcttctgacggggtgcgtttgctcccgtgcgggcaggcaacagaggtattcaccctggtcatgggg
agatcggcgatcgagctgggataagagatacggtcccgcgcaaggatcgctcatcctggtctgagccggacagtcattctggcaa
gcaatgacaacttgtcaggaccggaccgtgccatatatttctcacctagcgccgcaaaacctaacaatttgggagtcactgtgcca
ctgagttcgactggtagctgaatggagtcgctgctccactaaacgaattgtcagcaccgccagccggccgaggacccgagtcata

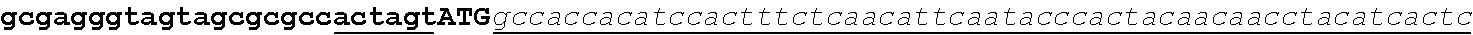

ggcgggctccgggccccggcgcccagcgaggcccctccccgtgcgcgggcgcgccatccccccccgcatcatcgtggtgtcctc
ctcctcctccaaggtgaaccccctgaagaccgaggccgtggtgtcctccggcctggccgaccgcctgcgcctgggctccctgacc
gaggacggcctgtcctacaaggagaagttcatcgtgcgctgctacgaggtgggcatcaacaagaccgccaccgtggagacc
atcgccaacctgctgcaggaggtgggctgcaaccacgcccagtccgtgggctactccaccgccggcttctccaccacccccacc
atgcgcaagctgcgcctgatctgggtgaccgcccgcatgcacatcgagatctacaagtaccccgcctggtccgacgtggtgga
gatcgagtcctggggccagggcgagggcaagatcggcacccgccgcgactggatcctgcgcgactacgccaccggccaggt
gatcggccgcgccacctccaagtgggtgatgatgaaccaggacacccgccgcctgcagaaggtggacgtggacgtgcgcga
cgagtacctggtgcactgcccccgcgagctgcgcctggccttccccgaggagaacaactcctccctgaagaagatctccaagct
ggaggacccctcccagtactccaagctgggcctggtgccccgccgcgccgacctggacatgaaccagcacgtgaacaacgtg
acctacatcggctgggtgctggagtccatgccccaggagatcatcgacacccacgagctgcagaccatcaccctggactaccg
ccgcgagtgccagcacgacgacgtggtggactccctgacctcccccgagccctccgaggacgccgaggccgtgttcaaccaca
acggcaccaacggctccgccaacgtgtccgccaacgaccacggctgccgcaacttcctgcacctgctgcgcctgtccggcaacg
gcctggagatcaaccgcggccgcaccgagtggcgcaagaagcccacccgcATGGACTACAAGGACCACGACGGCG

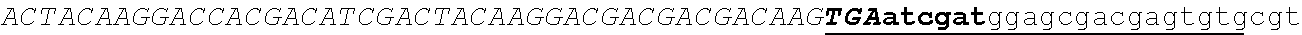

gcggggctggcgggagtgggacgccctcctcgctcctctctgttctgaacggaacaatcggccaccccgcgctacgcgccacgcatc
gagcaacgaagaaaaccccccgatgataggttgcggtggctgccgggatatagatccggccgcacatcaaagggcccctccgcca
gagaagaagctcctttcccagcagactccttctgctgccaaaacacttctctgtccacagcaacaccaaaggatgaacagatcaact
tgcgtctccgcgtagcttcctcggctagcgtgcttgcaacaggtccctgcactattatcttcctgctttcctctgaattatgcggcaggc
gagcgctcgctctggcgagcgctccttcgcgccgccctcgctgatcgagtgtacagtcaatgaatggtgagctccgcgtctcgaaca
gagcgcgcagaggaacgctgaaggtctcgcctctgtcgcacctcagcgcggcatacaccacaataaccacctgacgaatgcgcttg
gttcttcgtccattagcgaagcgtccggttcacacacgtgccacgttggcgaggtggcaggtgacaatgatcggtggagctgatggt

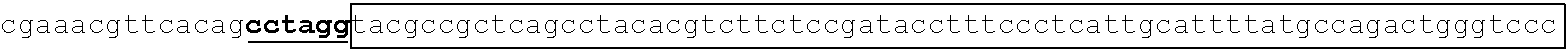

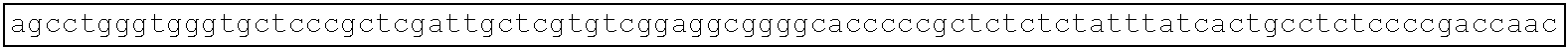

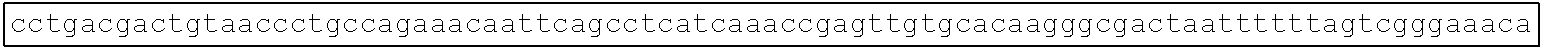

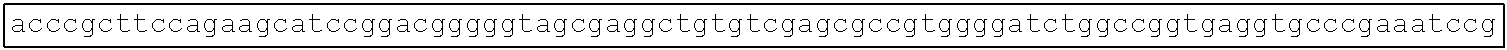

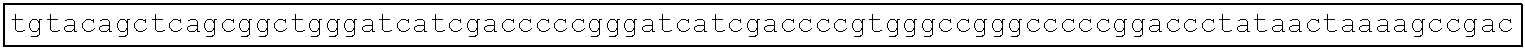

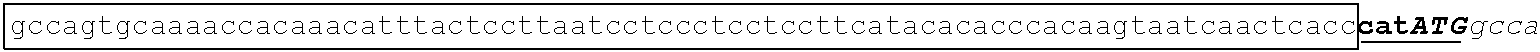

tcgccgccgccgccgtgatcgtgcccctgggcctgctgttcttcatctccggcctggtggtgaacctgatccaggccctgtgcttcg
tgctgatccgccccctgtccaagaacacctaccgcaagatcaaccgcgtggtggccgagctgctgtggctggagctgatctggc
tggtggactggtgggccggcgtgaagatcaaggtgttcatggaccccgagtccttcaacctgatgggcaaggagcacgccct
ggtggtggccaaccaccgctccgacatcgactggctggtgggctggctgctggcccagcgctccggctgcctgggctccgccct
ggccgtgatgaagaagtcctccaagttcctgcccgtgatcggctggtccatgtggttctccgagtacctgttcctggagcgctcct
gggccaaggacgagaacaccctgaaggccggcctgcagcgcctgaaggacttcccccgccccttctggctggccttcttcgtg
gagggcacccgcttcacccaggccaagttcctggccgcccaggagtacgccgcctcccagggcctgcccatcccccgcaacgt
gctgatcccccgcaccaagggcttcgtgtccgccgtgtcccacatgcgctccttcgtgcccgccatctacgacatgaccgtggcc
atccccaagtcctccccctcccccaccatgctgcgcctgttcaagggccagccctccgtggtgcacgtgcacatcaagcgctgcct
gatgaaggagctgcccgagaccgacgaggccgtggcccagtggtgcaaggacatgttcgtggagaaggacaagctgctgg
acaagcacatcgccgaggacaccactccgaccagcccatgcaggacctgggccgccccatcaagtccctgctggtggtggcc
tcctgggcctgcctgatggcctacggcgccctgaagttcctgcagtgctcctccctgctgtcctcctggaagggcatcgccacttc
ctggtgggcctggccatcgtgaccatcctgatgcacatcctgatcctgttctcccagtccgagcgctccacccccgccaaggtgg

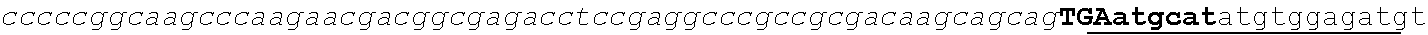

agggtggtcgactcgttggaggtgggtgtttttttttatcgagtgcgcggcgcggcaaacgggtccctttttatcgaggtgttcccaac
gccgcaccgccctcttaaaacaacccccaccaccacttgtcgaccttctcgtttgttatccgccacggcgccccggaggggcgtcgtc
tggccgcgcgggcagctgtatcgccgcgctcgctccaatggtgtgtaatcttggaaagataataatcgatggatgaggaggagagc
gtgggagatcagagcaaggaatatacagttggcacgaagcagcagcgtactaagctgtagcgtgttaagaaagaaaaactcgctg
ttaggctgtattaatcaaggagcgtatcaataattaccgaccctatacctttatctccaacccaatcgcggcttaaggatctaagtaa
gattcgaagcgctcgaccgtgccggacggactgcagccccatgtcgtagtgaccgccaatgtaagtgggctggcgtttccctgtacg
tgagtcaacgtcactgcacgcgcaccaccctctcgaccggcaggaccaggcatcgcgagatacagcgcgagccagacacggagtg

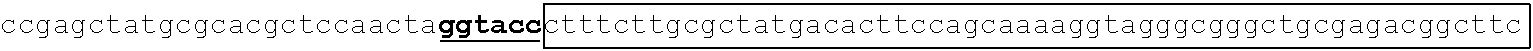

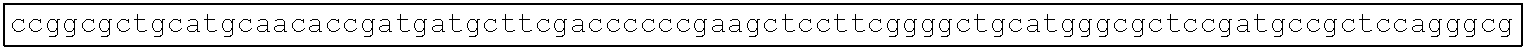

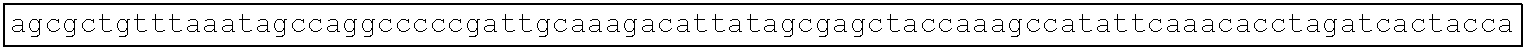

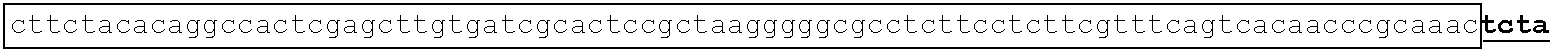

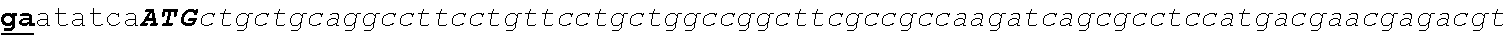

ccgaccgccccctggtgcacttcacccccaacaagggctggatgaacgaccccaacggcctgtggtacgacgagaaggacgc
caagtggcacctgtacttccagtacaacccgaacgacaccgtctgggggacgcccttgactggggccacgccacgtccgacg
acctgaccaactgggaggaccagcccatcgccatcgccccgaagcgcaacgactccggcgccttctccggctccatggtggtg
gactacaacaacacctccggcttcttcaacgacaccatcgacccgcgccagcgctgcgtggccatctggacctacaacaccccg
gagtccgaggagcagtacatctcctacagcctggacggcggctacaccttcaccgagtaccagaagaaccccgtgctggccg
ccaactccacccagaccgcgacccgaaggtcttctggtacgagccctcccagaagtggatcatgaccgcggccaagtcccag
gactacaagatcgagatctactcctccgacgacctgaagtcctggaagctggagtccgcgttcgccaacgagggcacctcgg
ctaccagtacgagtgccccggcctgatcgaggtccccaccgagcaggaccccagcaagtcctactgggtgatgttcatctccat
caaccccggcgccccggccggcggctccttcaaccagtacttcgtcggcagcttcaacggcacccacttcgaggccttcgacaa
ccagtcccgcgtggtggacttcggcaaggactactacgccctgcagaccttcttcaacaccgacccgacctacgggagcgccct
gggcatcgcgtgggcctccaactgggagtactccgccacgtgcccaccaacccctggcgctcctccatgtccctcgtgcgcaag
ttctccctcaacaccgagtaccaggccaacccggagacggagctgatcaacctgaaggccgagccgatcctgaacatcagca
acgccggcccctggagccggttcgccaccaacaccacgttgacgaaggccaacagctacaacgtcgacctgtccaacagcac
cggcaccctggagttcgagctggtgtacgccgtcaacaccacccagacgatctccaagtccgtgttcgcggacctctccctctgg
ttcaagggcctggaggaccccgaggagtacctccgcatgggcttcgaggtgtccgcgtcctccttcttcctggaccgcgggaac
agcaaggtgaagttcgtgaaggagaacccctacttcaccaaccgcatgagcgtgaacaaccagcccttcaagagcgagaac
gacctgtcctactacaaggtgtacggcttgctggaccagaacatcctggagctgtacttcaacgacggcgacgtcgtgtccacc
aacacctacttcatgaccaccgggaacgccctgggctccgtgaacatgacgacgggggtggacaacctgttctacatcgaca

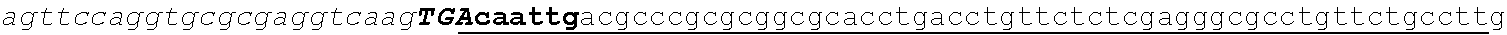

cgaaacaagcccctggagcatgcgtgcatgatcgtctctggcgccccgccgcgcggtttgtcgccctcgcgggcgccgcggccgcg
ggggcgcattgaaattgttgcaaaccccacctgacagattgagggcccaggcaggaaggcgttgagatggaggtacaggagtcaa
gtaactgaaagtttttatgataactaacaacaaagggtcgtttctggccagcgaatgacaagaacaagattccacatttccgtgtag
aggcttgccatcgaatgtgagcgggcgggccgcggacccgacaaaacccttacgacgtggtaagaaaaacgtggcgggcactgtc
cctgtagcctgaagaccagcaggagacgatcggaagcatcacagcacaggatcctgaggacagggtggttggctggatggggaa
acgctggtcgcgggattcgatcctgctgcttatatcctccctggaagcacacccacgactctgaagaagaaaacgtgcacacaca
caacccaaccggccgaatatttgcttccttatcccgggtccaagagagactgcgatgcccccctcaatcagcatcctcctccctgcc
gcttcaatcttccctgcttgcctgcgcccgcggtgcgccgtctgcccgcccagtcagtcactcctgcacaggccccttgtgcgcagtg
ctcctgtaccctttaccgctccttccattctgcgaggccccctattgaatgtattcgttgcctgtgtggccaagcgggctgctgggcgc
gccgccgtcgggcagtgctcggcgactttggcggaagccgattgttcttctgtaagccacgcgcttgctgctttgggaagagaagg
gggggggtactgaatggatgaggaggagaaggaggggtattggtattatctgagttggggaggcagggagagttggaaaatgt
aagtggcacgacgggcaaggagaatggtgagcatgtgcatggtgatgtcgttggtcgaggacgatcctgcacgcgtgtatctgat
gtagaatacggcaatcaccctagtctacatctataccttctccgtataacgccctttccaaatgccctcccgtttctctcctattcttg
atccacatgatgaccctggcactatttcaagggctggagaagagcgtttaaac

Construct pSZ5868 was transformed into S8754. Primary transformants were clonally purified and screened under standard lipid production conditions at pH 5. Integration of pSZ5868 at the FATA-1 locus was verified by DNA blot analysis. The fatty acid profiles and lipid titers of lead strains were assayed in 50-mL shake flasks (Table 22). S8813 was selected as the lead strain for the final round of genetic engineering. As shown in Table 22 as compared to strain S8754, C16:0 decreased from 5.9% to 3.4%, and C18:0 increased from 27.3% to about 45%. C18:2 increased slightly from 1.3% to about 1.6% due to the activity of the T. cacao LPAAT.

TABLE 22
Fatty acid profiles of FATA-1 ablation strains.

| Strain | 55100 | 58754 | 58813 | 58814 |
|---|---|---|---|---|
| C14:0 | 0.7 | 0.6 | 0.5 | 0.5 |
| C16:0 | 18.8 | 5.9 | 3.4 | 3.4 |
| C16:1 cis-9 | 0.5 | 0.0 | 0.0 | 0.0 |
| C18:0 | 4.0 | 27.3 | 45.3 | 44.8 |
| C18:1 | 68.3 | 60.9 | 45.9 | 46.3 |
| C18:2 | 6.3 | 1.3 | 1.5 | 1.6 |
| C18:3 α | 0.6 | 0.3 | 0.3 | 0.3 |
| C20:0 | 0.3 | 2.4 | 2.0 | 2.1 |
| saturates | 24.2 | 37.0 | 52.0 | 51.5 |
| lipid (g/L) | 12.7 | 11.9 | 11.9 | 11.9 |

### Constructs Used for FAD2 Knockout in S8813

The high-SOS strains were generated by transformation of strain S8813 with integrative plasmid pSZ6383 (FAD2-1vA::PmLDH1-AtTHIC-PmHSP90:PmSAD2-2v2-TcDGAT1-CvNR:PmSAD2-1v3-CpSAD1tp_GarmFATA1(G108A)_FLAG-PmSAD2-1::FAD2-1vB), plasmid pSZ6384 (FAD2-1vA::PmLDH1-AtTHIC-PmHSP90:PmSAD2-2v2-TcDGAT2-CvNR:PmSAD2-1v3-CpSAD1tp_GarmFATA1(G108A)_FLAG-PmSAD2-1::FAD2-1vB), or plasmid pSZ6377 (FAD2-1vA::PmLDH1-AtTHIC-PmHSP90: PmSAD2-1v3-CpSAD1tp_GarmFATA1(G108A)_FLAG-PmSAD2-1::FAD2-1vB). These constructs targeted ablation of allele 1 of the endogenous fatty acid desaturase 2 gene (FAD2-1), and contained expression modules for a second copy of GarmFATA1(G108A), and either TcDGAT1 encoding the Theobroma cacao diacylglycerol O-acyltransferase 1 (pSZ6383) or TcDGAT2 encoding the Theobroma cacao diacylglycerol O-acyltransferase 2 (pSZ6384). Deletion of one allele of FAD2 further reduced C18:2 accumulation. Expression of GarmFATA1(G108A) stimulated C18:0-ACP hydrolysis, further increasing C18:0. TcDGAT1 and TcDGAT2 had superior specificity for transfer of C18:0 to the sn-3 position of triacylglycerides than the endogeneous DGAT, leading to an increase in C18:0 and lipid titer, and a reduction in trisaturated TAGs. The final strains had higher C18:0, lower C16:0 and lower C18:2 than their parent, S8813. The Arabidopsis thaliana THIC gene (AtTHIC) catalyzes the conversion of 5-aminoimidazole ribotide (AIR) to 4-amino-5-hydroxymethylpyrimidine (HMP), providing the pyrimidine ring structure for the biosynthesis of thiamine. AtTHIC served as a selectable marker as part of plasmids pSZ6383 and pSZ6384, allowing the strains to grow in the absence of exogenous thiamine.

The sequence of the pSZ6383 transforming DNA is provided below. Relevant restriction sites in the construct are indicated in lowercase, bold and underlined text, and are 5′-3′ BspQI, KpnI, XbaI, SnaBI, BamHI, AvrII, SpeI, ClaI, AflII, EcoRI, SpeI, AscI, ClaI, SacI and BspQ I, respectively. BspQI sites delimit the 5′ and 3′ ends of the transforming DNA. Proceeding in the 5′ to 3′ direction, bold, lowercase sequences represent FAD2-1 5′ genomic DNA that permits targeted integration at the FAD2-1 locus via homologous recombination. The P. moriformis LDH1 promoter driving the expression of the Arabidopsis thaliana THIC gene is indicated by boxed text. The initiator ATG and terminator TGA for AtTHIC are indicated by uppercase, bold italics while the coding region is indicated in lowercase italics. The P. moriformis HSP90 3′ UTR is indicated by lowercase underlined text. A spacer sequence is represented by lowercase text. The P. moriformis SAD2-2 promoter, driving expression of the TcDGAT1 sequence is indicated by boxed text. The initiator ATG and terminator TGA codons of the TcDGAT1 gene are indicated by uppercase, bold italics, while the remainder of the coding region is represented with italics. Lowercase underlined text represents the C. vulgaris NR 3′ UTR. A second spacer sequence is represented by lowercase text. The P. moriformis SAD2-1 promoter, indicated by boxed italicized text, is utilized to drive the expression of the G. mangostana FATA1 gene. The initiator ATG of the sequence encoding the C. protothecoides SAD1 transit peptide (CpSAD1tp) is indicated by uppercase, bold italics, and the remainder of the CpSAD1tp sequence located between the ATG and the AscI site is indicated with lowercase, underlined italics. The GarmFATA1(G108A) coding region is indicated by lowercase italics. A sequence encoding a 3×FLAG tag fused to the C-terminus of GarmFATA1(G108A) is represented by uppercase italics, and the TGA terminator codon is indicated with uppercase, bold italics. The P. moriformis SAD2-1 3′ UTR is indicated by lowercase underlined text. The FAD2-1 3′ genomic region is indicated by bold, lowercase text.

Nucleotide sequence of transforming DNA contained in pSZ6383
SEQ ID NO: 128
gctcttcgcgaaggtcattttccagaacaacgaccatggcttgtcttagcgatcgctcgaatgactgctagtgagtcgtacgctcga
cccagtcgctcgcaggagaacgcggcaactgccgagcttcggcttgccagtcgtgactcgtatgtgatcaggaatcattggcattg
gtagcattataattcggcttccgcgctgtttatgggcatggcaatgtctcatgcagtcgaccttagtcaaccaattctgggtggccag
ctccgggcgaccgggctccgtgtcgccgggcaccacctcctgccatgagtaacagggccgccctctcctcccgacgttggccaact
gaataccgtgtcttggggccctacatgatgggctgcctagtcgggcgggacgcgcaactgcccgcgcaatctgggacgtggtctga
atcctccaggcgggtttccccgagaaagaaagggtgccgatttcaaagcagagccatgtgccgggccctgtggcctgtgttggcgc
ctatgtagtcaccccccctcacccaattgtcgccagtttgcgcaatccataaactcaaaactgcagcttctgagctgcgctgttcaa

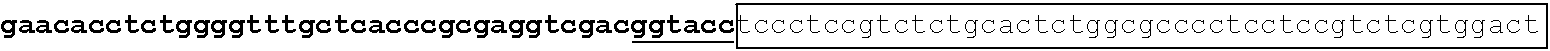

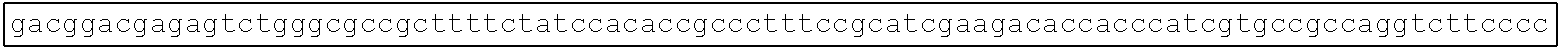

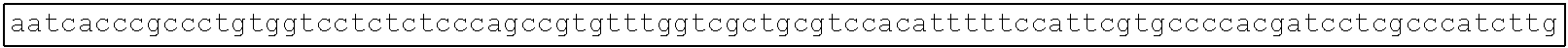

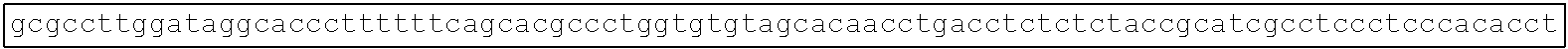

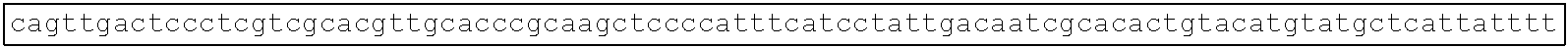

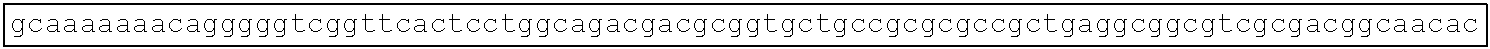

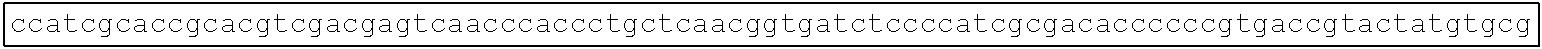

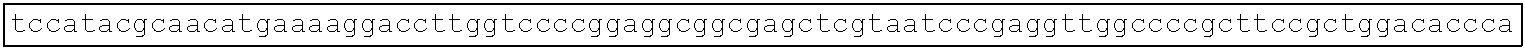

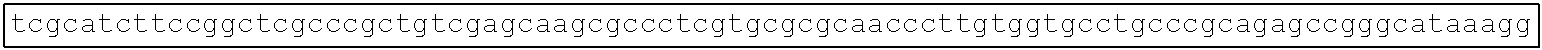

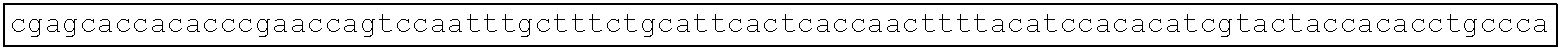

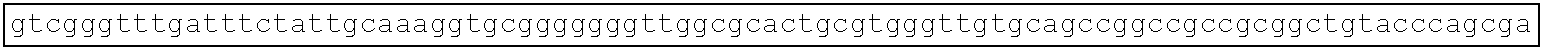

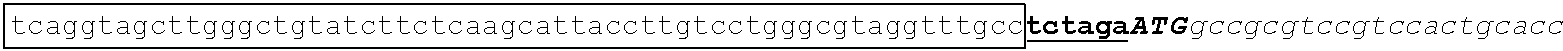

ctgatgtccgtggtctgcaacaacaagaaccactccgcccgccccaagctgcccaactcctccctgctgcccggcttcgacgtgg
tggtccaggccgcggccacccgcttcaagaaggagacgacgaccacccgcgccacgctgacgttcgacccccccacgaccaa
ctccgagcgcgccaagcagcgcaagcacaccatcgacccctcctcccccgacttccagcccatcccctccttcgaggagtgcttc
cccaagtccacgaaggagcacaaggaggtggtgcacgaggagtccggccacgtcctgaaggtgcccttccgccgcgtgcac
ctgtccggcggcgagcccgccttcgacaactacgacacgtccggcccccagaacgtcaacgcccacatcggcctggcgaagct
gcgcaaggagtggatcgaccgccgcgagaagctgggcacgccccgctacacgcagatgtactacgcgaagcagggcatcat
cacggaggagatgctgtactgcgcgacgcgcgagaagctggaccccgagttcgtccgctccgaggtcgcgcggggccgcgc
catcatcccctccaacaagaagcacctggagctggagcccatgatcgtgggccgcaagttcctggtgaaggtgaacgcgaac
atcggcaactccgccgtggcctcctccatcgaggaggaggtctacaaggtgcagtgggccaccatgtggggcgccgacacca
tcatggacctgtccacgggccgccacatccacgagacgcgcgagtggatcctgcgcaactccgcggtccccgtgggcaccgtc
cccatctaccaggcgctggagaaggtggacggcatcgcggagaacctgaactgggaggtgttccgcgagacgctgatcgag
caggccgagcagggcgtggactacttcacgatccacgcgggcgtgctgctgcgctacatccccctgaccgccaagcgcctgac
gggcatcgtgtcccgcggcggctccatccacgcgaagtggtgcctggcctaccacaaggagaacttcgcctacgagcactggg
acgacatcctggacatctgcaaccagtacgacgtcgccctgtccatcggcgacggcctgcgccccggctccatctacgacgcca
acgacacggcccagttcgccgagctgctgacccagggcgagctgacgcgccgcgcgtgggagaaggacgtgcaggtgatg
aacgagggccccggccacgtgcccatgcacaagatccccgagaacatgcagaagcagctggagtggtgcaacgaggcgcc
cttctacaccctgggccccctgacgaccgacatcgcgcccggctacgaccacatcacctccgccatcggcgcggccaacatcgg
cgccctgggcaccgccctgctgtgctacgtgacgcccaaggagcacctgggcctgcccaaccgcgacgacgtgaaggcgggc
gtcatcgcctacaagatcgccgcccacgcggccgacctggccaagcagcacccccacgcccaggcgtgggacgacgcgctgt
ccaaggcgcgcttcgagttccgctggatggaccagttcgcgctgtccctggaccccatgacggcgatgtccttccacgacgaga
cgctgcccgcggacggcgcgaaggtcgcccacttctgctccatgtgcggccccaagttctgctccatgaagatcacggaggac
atccgcaagtacgccgaggagaacggctacggctccgccgaggaggccatccgccagggcatggacgccatgtccgagga
gttcaacatcgccaagaagacgatctccggcgagcagcacggcgaggtcggcggcgagatctacctgcccgagtcctacgtc

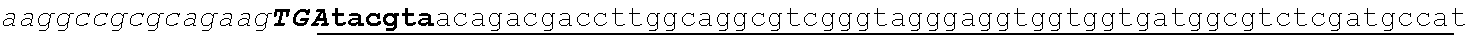

cgcacgcatccaacgaccgtatacgcatcgtccaatgaccgtcggtgtcctctctgcctccgttttgtgagatgtctcaggcttggtgc
atcctcgggtggccagccacgttgcgcgtcgtgctgcttgcctctcttgcgcctctgtggtactggaaaatatcatcgaggcccgttttt
ttgctcccatttcctttccgctacatcttgaaagcaaacgacaaacgaagcagcaagcaaagagcacgaggacggtgaacaagtct
gtcacctgtatacatctatttccccgcgggtgcacctactctctctcctgccccggcagagtcagctgccttacgtgacggatcccgcg
tctcgaacagagcgcgcagaggaacgctgaaggtctcgcctctgtcgcacctcagcgcggcatacaccacaataaccacctgacga
atgcgcttggttcttcgtccattagcgaagcgtccggttcacacacgtgccacgttggcgaggtggcaggtgacaatgatcggtgga

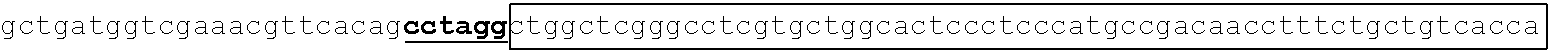

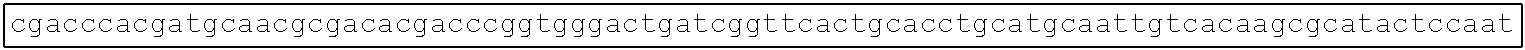

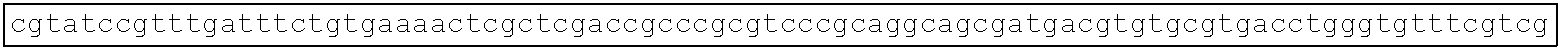

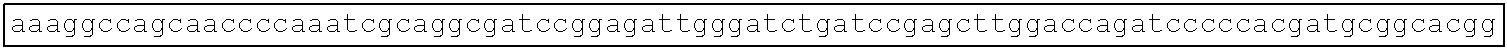

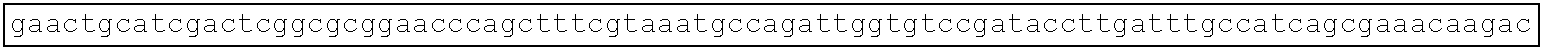

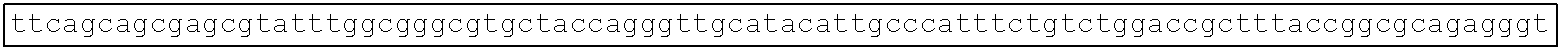

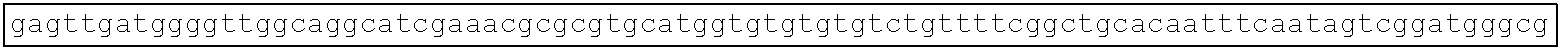

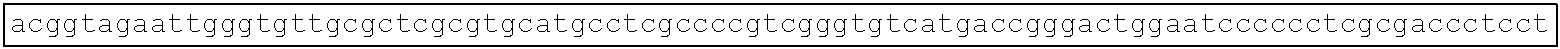

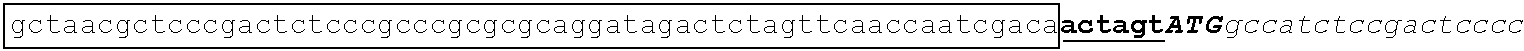

cgagatcctgggctccaccgccaccgtgacctcctcctcccactccgactccgacctgaacctgctgtccatccgccgccgcacct
ccaccaccgccgccgcccgcgcccccgaccgcgacgactccggcaacggcgaggccgtggacgaccgcgaccgcgtggagt
ccgccaacctgatgtccaacgtggccgagaacgccaacgagatgcccaactcctccgacacccgcttcacctaccgcccccgcg
tgcccgcccaccgccgcatcaaggagtcccccctgtcctccggcgccatcttcaagcagtcccacgccggcctgttcaacctgtgc
atcgtggtgctggtggccgtgaactcccgcctgatcatcgagaacctgatgaagtacggctggctgatccgctccggcttctggt
tctcctcccgctccctgtccgactggcccctgttcatgtgctgcctgaccctgcccatcttccccctggccgccttcgtggtggagaa
gctggtgcagcgcaactacatctccgagcccgtggtggtgttcctgcacgccatcatctccaccaccgccgtgctgtaccccgtg
atcgtgaacctgcgctgcgactccgccttcctgtccggcgtggccctgatgctgttcgcctgcatcgtgtggctgaagctggtgtc
ctacgcccacaccaacaacgacatgcgcgccctggccaagtccgccgagaagggcgacgtggacccctcctacgacgtgtcct
tcaagtccctggcctacttcatggtggcccccaccctgtgctaccagcagtcctacccccgcacccccgccgtgcgcaagtcctgg
gtggtgcgccagttcatcaagctgatcgtgttcaccggcctgatgggcttcatcatcgagcagtacatcaaccccatcgtgcag
aactcccagcaccccctgaagggcaacctgctgtacgccatcgagcgcgtgctgaagctgtccgtgcccaacctgtacgtgtgg
ctgtgcatgttctactgcttcttccacctgtggctgaacatcctggccgagctgctgcgcttcggcgaccgcgagttctacaagga
ctggtggaacgccaagaccgtggaggagtactggcgcatgtggaacatgcccgtgcacaagtggatggtgcgccacatctac
ttcccctgcctgcgcaacggcatccccaagggcgtggccatcgtgatcgccttcctggtgtccgccgtgttccacgagctgtgcat
cgccgtgccctgccacatgttcaagctgtgggccttcatcggcatcatgttccaggtgcccctggtgctgatcaccaactacctgc
aggacaagttccgctcctccatggtgggcaacatgatcttctggttcatcttctccatcctgggccagcccatgtgcgtgctgctgt

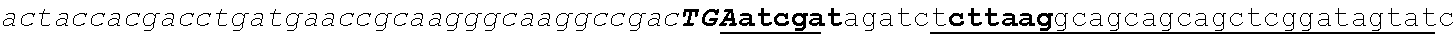

gacacactctggacgctggtcgtgtgatggactgttgccgccacacttgctgccttgacctgtgaatatccctgccgcttttatcaaac
agcctcagtgtgtttgatcttgtgtgtacgcgcttttgcgagttgctagctgcttgtgctatttgcgaataccacccccagcatccccttc
cctcgtttcatatcgcttgcatcccaaccgcaacttatctacgctgtcctgctatccctcagcgctgctcctgctcctgctcactgcccct
cgcacagccttggtttgggctccgcctgtattctcctggtactgcaacctgtaaaccagcactgcaatgctgatgcacgggaagtagt
gggatgggaacacaaatggacttaaggatctaagtaagattcgaagcgctcgaccgtgccggacggactgcagccccatgtcgta
gtgaccgccaatgtaagtgggctggcgtttccctgtacgtgagtcaacgtcactgcacgcgcaccaccctctcgaccggcaggacca
ggcatcgcgagatacagcgcgagccagacacggagtgccgagctatgcgcacgctccaactagatatcatgtggatgatgagcat

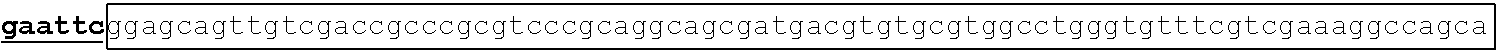

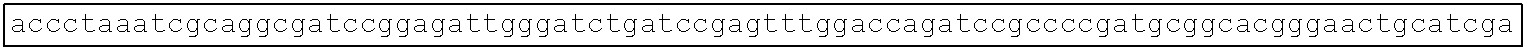

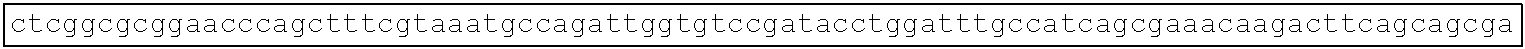

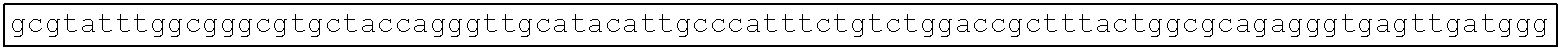

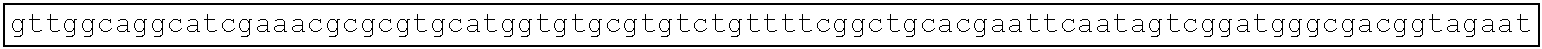

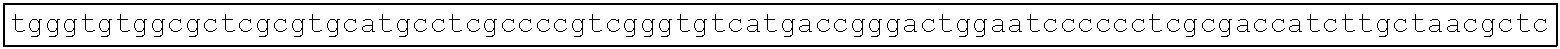

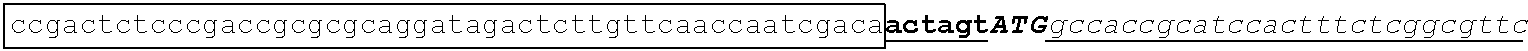

aatgcccactgcggcgacctgcgtcgctcggcgggctccgggccccggcgcccagcgaggcccctccccgtgcgcgggcgcgc
catccccccccgcatcatcgtggtgtcctcctcctcctccaaggtgaaccccctgaagaccgaggccgtggtgtcctccggcctgg
ccgaccgcctgcgcctgggctccctgaccgaggacggcctgtcctacaaggagaagttcatcgtgcgctgctacgaggtgggc
atcaacaagaccgccaccgtggagaccatcgccaacctgctgcaggaggtgggctgcaaccacgcccagtccgtgggctact
ccaccgccggcttctccaccacccccaccatgcgcaagctgcgcctgatctgggtgaccgcccgcatgcacatcgagatctaca
agtaccccgcctggtccgacgtggtggagatcgagtcctggggccagggcgagggcaagatcggcacccgccgcgactgga
tcctgcgcgactacgccaccggccaggtgatcggccgcgccacctccaagtgggtgatgatgaaccaggacacccgccgcctg
cagaaggtggacgtggacgtgcgcgacgagtacctggtgcactgcccccgcgagctgcgcctggccttccccgaggagaaca
actcctccctgaagaagatctccaagctggaggacccctcccagtactccaagctgggcctggtgccccgccgcgccgacctgg
acatgaaccagcacgtgaacaacgtgacctacatcggctgggtgctggagtccatgccccaggagatcatcgacacccacga
gctgcagaccatcaccctggactaccgccgcgagtgccagcacgacgacgtggtggactccctgacctcccccgagccctccg
aggacgccgaggccgtgttcaaccacaacggcaccaacggctccgccaacgtgtccgccaacgaccacggctgccgcaactt
cctgcacctgctgcgcctgtccggcaacggcctggagatcaaccgcggccgcaccgagtggcgcaagaagcccacccgcAT
GGACTACAAGGACCACGACGGCGACTACAAGGACCACGACATCGACTACAAGGACGACGACGACAA

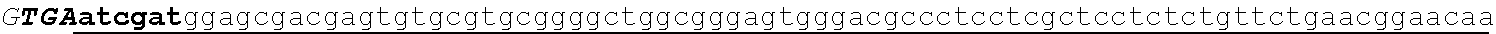

tcggccaccccgcgctacgcgccacgcatcgagcaacgaagaaaaccccccgatgataggttgcggtggctgccgggatatagat
ccggccgcacatcaaagggcccctccgccagagaagaagctcctttcccagcagactccttctgctgccaaaacacttctctgtcca
cagcaacaccaaaggatgaacagatcaacttgcgtctccgcgtagcttcctcggctagcgtgcttgcaacaggtccctgcactattat
cttcctgctttcctctgaattatgcggcaggcgagcgctcgctctggcgagcgctccttcgcgccgccctcgctgatcgagtgtacagt
caatgaatggtgagctcctcactcagcgcgcctgcgcggggatgcggaacgccgccgccgccttgtcttttgcacgcgcgactccgt
cgcttcgcgggtggcacccccattgaaaaaaacctcaattctgtttgtggaagacacggtgtacccccaaccacccacctgcacct
ctattattggtattattgacgcgggagcgggcgttgtactctacaacgtagcgtctctggttttcagctggctcccaccattgtaaatt
cttgctaaaatagtgcgtggttatgtgagaggtatggtgtaacagggcgtcagtcatgttggttttcgtgctgatctcgggcacaag
gcgtcgtcgacgtgacgtgcccgtgatgagagcaataccgcgctcaaagccgacgcatggcctttactccgcactccaaacgact
gtcgctcgtatttttcggatatctattttttaagagcgagcacagcgccgggcatgggcctgaaaggcctcgcggccgtgctcgtgg
tgggggccgcgagcgcgtggggcatcgcggcagtgcaccaggcgcagacggaggaacgcatggtgagtgcgcatcacaagatg
catgtcttgttgtctgtactataatgctagagcatcaccaggggcttagtcatcgcacctgctttggtcattacagaaattgcacaag
ggcgtcctccgggatgaggagatgtaccagctcaagctggagcggcttcgagccaagcaggagcgcggcgcatgacgacctacc
cacatgcgaagagc

The sequence of the pSZ6384 transforming DNA is provided below. Relevant restriction sites in the construct are indicated in lowercase, bold and underlined text, and are 5′-3′ BspQI, KpnI, XbaI, SnaBI, BamHI, AvrII, SpeI, ClaI, AfIII, EcoRI, SpeI, AscI, ClaI, SacI and BspQ I, respectively. BspQI sites delimit the 5′ and 3′ ends of the transforming DNA. Proceeding in the 5′ to 3′ direction, bold, lowercase sequences represent FAD2-1 5′ genomic DNA that permits targeted integration at the FAD2-1 locus via homologous recombination. The P. moriformis LDH1 promoter driving the expression of the Arabidopsis thaliana THIC gene is indicated by boxed text. The initiator ATG and terminator TGA for AtTHIC are indicated by uppercase, bold italics while the coding region is indicated in lowercase italics. The P. moriformis HSP90 3′ UTR is indicated by lowercase underlined text. A spacer sequence is represented by lowercase text. The P. moriformis SAD2-2 promoter, driving expression of the TcDGAT2 sequence is indicated by boxed text. The initiator ATG and terminator TGA codons of the TcDGAT2 gene are indicated by uppercase, bold italics, while the remainder of the coding region is represented with italics. Lowercase underlined text represents the C. vulgaris NR 3′ UTR. A second spacer sequence is represented by lowercase text. The P. moriformis SAD2-1 promoter, indicated by boxed italicized text, is utilized to drive the expression of the G. mangostana FATA1 gene. The initiator ATG of the sequence encoding the C. protothecoides SAD1 transit peptide (CpSAD1tp) is indicated by uppercase, bold italics, and the remainder of the CpSAD1tp sequence located between the ATG and the AscI site is indicated with lowercase, underlined italics. The GarmFATA1(G108A) coding region is indicated by lowercase italics. A sequence encoding a 3×FLAG tag fused to the C-terminus of GarmFATA1(G108A) is represented by uppercase italics, and the TGA terminator codon is indicated with uppercase, bold italics. The P. moriformis SAD2-1 3′ UTR is indicated by lowercase underlined text. The FAD2-1 3′ genomic region is indicated by bold, lowercase text.

Nucleotide sequence of transforming DNA contained in pSZ6384
SEQ ID NO: 129
gctcttcgcgaaggtcattttccagaacaacgaccatggcttgtcttagcgatcgctcgaatgactgctagtgagtcgtacgctcga
cccagtcgctcgcaggagaacgcggcaactgccgagcttcggcttgccagtcgtgactcgtatgtgatcaggaatcattggcattg
gtagcattataattcggcttccgcgctgtttatgggcatggcaatgtctcatgcagtcgaccttagtcaaccaattctgggtggccag
ctccgggcgaccgggctccgtgtcgccgggcaccacctcctgccatgagtaacagggccgccctctcctcccgacgttggccaact
gaataccgtgtcttggggccctacatgatgggctgcctagtcgggcgggacgcgcaactgcccgcgcaatctgggacgtggtctga
atcctccaggcgggtttccccgagaaagaaagggtgccgatttcaaagcagagccatgtgccgggccctgtggcctgtgttggcgc
ctatgtagtcaccccccctcacccaattgtcgccagtttgcgcaatccataaactcaaaactgcagcttctgagctgcgctgttcaa

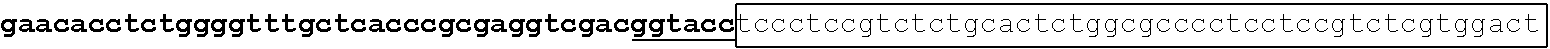

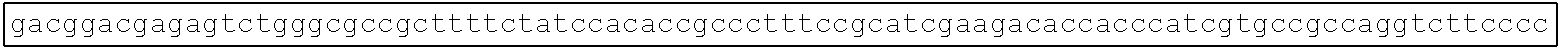

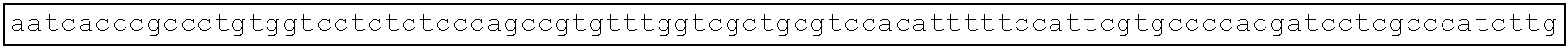

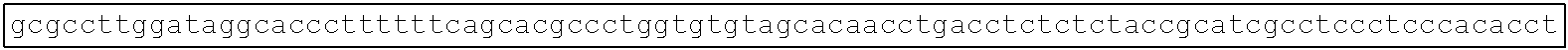

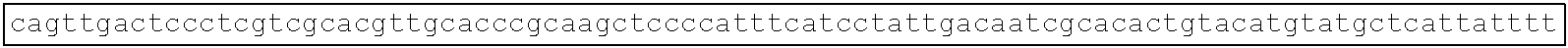

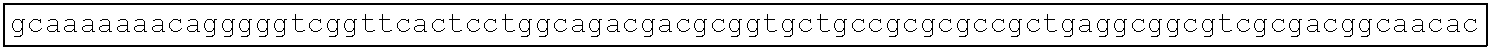

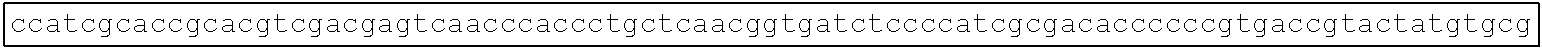

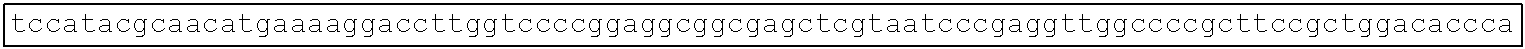

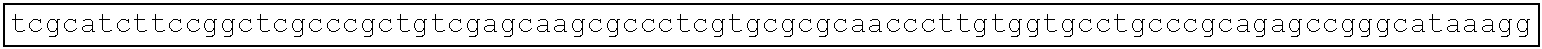

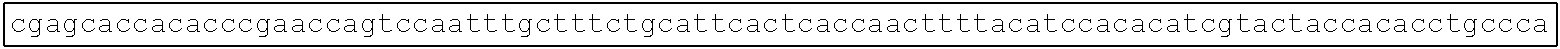

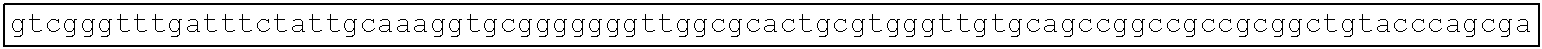

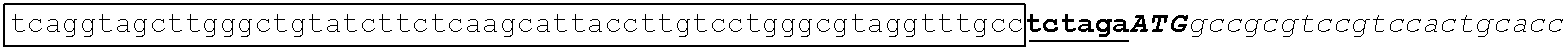

ctgatgtccgtggtctgcaacaacaagaaccactccgcccgccccaagctgcccaactcctccctgctgcccggcttcgacgtgg
tggtccaggccgcggccacccgcttcaagaaggagacgacgaccacccgcgccacgctgacgttcgacccccccacgaccaa
ctccgagcgcgccaagcagcgcaagcacaccatcgacccctcctcccccgacttccagcccatcccctccttcgaggagtgcttc
cccaagtccacgaaggagcacaaggaggtggtgcacgaggagtccggccacgtcctgaaggtgcccttccgccgcgtgcac
ctgtccggcggcgagcccgccttcgacaactacgacacgtccggcccccagaacgtcaacgcccacatcggcctggcgaagct
gcgcaaggagtggatcgaccgccgcgagaagctgggcacgccccgctacacgcagatgtactacgcgaagcagggcatcat
cacggaggagatgctgtactgcgcgacgcgcgagaagctggaccccgagttcgtccgctccgaggtcgcgcggggccgcgc
catcatcccctccaacaagaagcacctggagctggagcccatgatcgtgggccgcaagttcctggtgaaggtgaacgcgaac
atcggcaactccgccgtggcctcctccatcgaggaggaggtctacaaggtgcagtgggccaccatgtggggcgccgacacca
tcatggacctgtccacgggccgccacatccacgagacgcgcgagtggatcctgcgcaactccgcggtccccgtgggcaccgtc
cccatctaccaggcgctggagaaggtggacggcatcgcggagaacctgaactgggaggtgttccgcgagacgctgatcgag
caggccgagcagggcgtggactacttcacgatccacgcgggcgtgctgctgcgctacatccccctgaccgccaagcgcctgac
gggcatcgtgtcccgcggcggctccatccacgcgaagtggtgcctggcctaccacaaggagaacttcgcctacgagcactggg
acgacatcctggacatctgcaaccagtacgacgtcgccctgtccatcggcgacggcctgcgccccggctccatctacgacgcca
acgacacggcccagttcgccgagctgctgacccagggcgagctgacgcgccgcgcgtgggagaaggacgtgcaggtgatg
aacgagggccccggccacgtgcccatgcacaagatccccgagaacatgcagaagcagctggagtggtgcaacgaggcgcc
cttctacaccctgggccccctgacgaccgacatcgcgcccggctacgaccacatcacctccgccatcggcgcggccaacatcgg
cgccctgggcaccgccctgctgtgctacgtgacgcccaaggagcacctgggcctgcccaaccgcgacgacgtgaaggcgggc
gtcatcgcctacaagatcgccgcccacgcggccgacctggccaagcagcacccccacgcccaggcgtgggacgacgcgctgt
ccaaggcgcgcttcgagttccgctggatggaccagttcgcgctgtccctggaccccatgacggcgatgtccttccacgacgaga
cgctgcccgcggacggcgcgaaggtcgcccacttctgctccatgtgcggccccaagttctgctccatgaagatcacggaggac
atccgcaagtacgccgaggagaacggctacggctccgccgaggaggccatccgccagggcatggacgccatgtccgagga
gttcaacatcgccaagaagacgatctccggcgagcagcacggcgaggtcggcggcgagatctacctgcccgagtcctacgtc

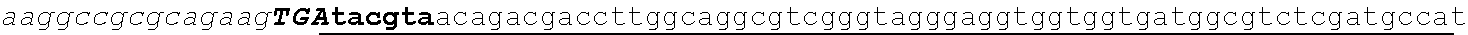

cgcacgcatccaacgaccgtatacgcatcgtccaatgaccgtcggtgtcctctctgcctccgttttgtgagatgtctcaggcttggtgc
atcctcgggtggccagccacgttgcgcgtcgtgctgcttgcctctcttgcgcctctgtggtactggaaaatatcatcgaggcccgttttt
ttgctcccatttcctttccgctacatcttgaaagcaaacgacaaacgaagcagcaagcaaagagcacgaggacggtgaacaagtct
gtcacctgtatacatctatttccccgcgggtgcacctactctctctcctgccccggcagagtcagctgccttacgtgacggatcccgcg
tctcgaacagagcgcgcagaggaacgctgaaggtctcgcctctgtcgcacctcagcgcggcatacaccacaataaccacctgacga
atgcgcttggttcttcgtccattagcgaagcgtccggttcacacacgtgccacgttggcgaggtggcaggtgacaatgatcggtgga

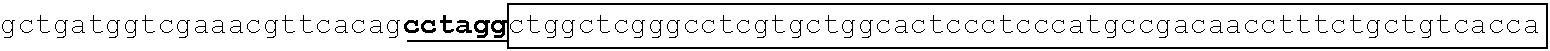

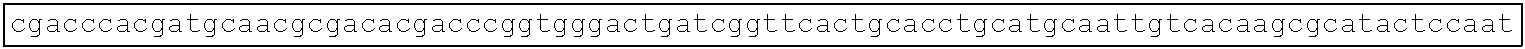

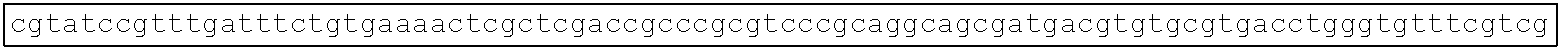

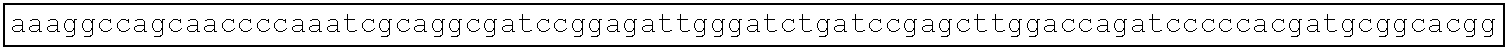

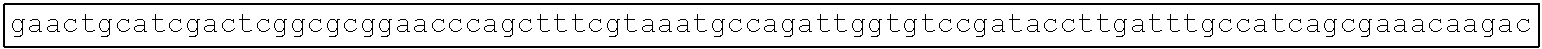

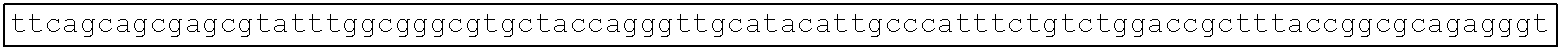

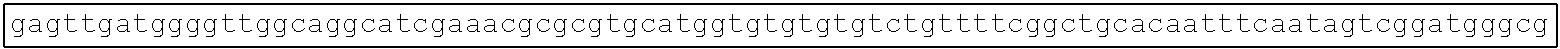

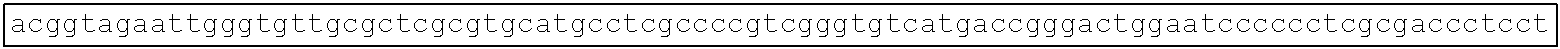

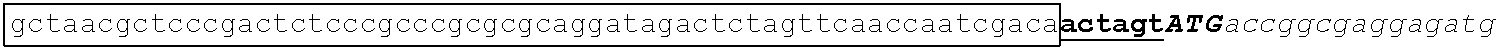

gaggagcgcaaggccaccggctaccgcgagactccggccgccacgagacccctccaacaccatgcacgccctgctggccat
gggcatctggctgggcgccatccacttcaacgccctgctgctgctgactccttcctgacctgcccttctccaagttcctggtggtgt
tcggcctgctgctgctgacatgatcctgcccatcgacccctactccaagttcggccgccgcctgtcccgctacatctccaagcacg
cctgctcctacttccccatcaccctgcacgtggaggacatccacgccttccaccccgaccgcgcctacgtgttcggcttcgagccc
cactccgtgctgcccatcggcgtggtggccctggccgacctgaccggcttcatgcccctgcccaagatcaaggtgctggcctcct
ccgccgtgttctacacccccacctgcgccacatctggacctggctgggcctgacccccgccaccaagaagaacttctcctccctg
ctggacgccggctactcctgcatcctggtgcccggcggcgtgcaggagaccaccacatggagcccggctccgagatcgccttc
ctgcgcgcccgccgcggcttcgtgcgcatcgccatggagatgggctcccccctggtgcccgtgttctgcttcggccagtcccacgt
gtacaagtggtggaagcccggcggcaagttctacctgcagttctcccgcgccatcaagttcacccccatcttcttctggggcatct
tcggctcccccctgccctaccagcaccccatgcacgtggtggtgggcaagcccatcgacgtgaagaagaacccccagcccatc
gtggaggaggtgatcgaggtgcacgaccgcttcgtggaggccctgcaggacctgttcgagcgccacaaggcccaggtgggc

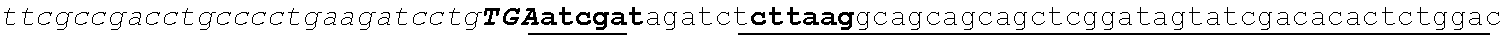

gctggtcgtgtgatggactgttgccgccacacttgctgccttgacctgtgaatatccctgccgcttttatcaaacagcctcagtgtgttt
gatcttgtgtgtacgcgcttttgcgagttgctagctgcttgtgctatttgcgaataccacccccagcatccccttccctcgtttcatatcg
cttgcatcccaaccgcaacttatctacgctgtcctgctatccctcagcgctgctcctgctcctgctcactgcccctcgcacagccttggt
ttgggctccgcctgtattctcctggtactgcaacctgtaaaccagcactgcaatgctgatgcacgggaagtagtgggatgggaacac
aaatggacttaaggatctaagtaagattcgaagcgctcgaccgtgccggacggactgcagccccatgtcgtagtgaccgccaatgt
aagtgggctggcgtttccctgtacgtgagtcaacgtcactgcacgcgcaccaccctctcgaccggcaggaccaggcatcgcgagat

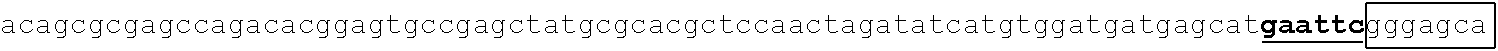

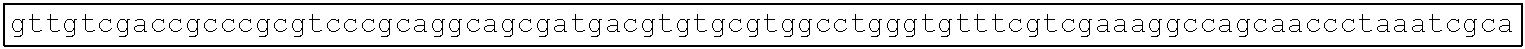

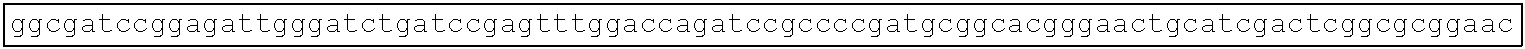

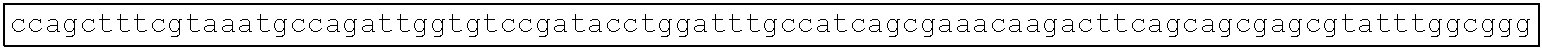

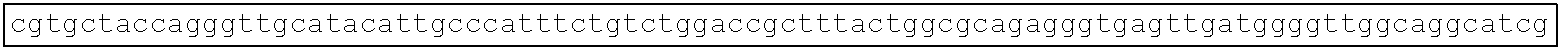

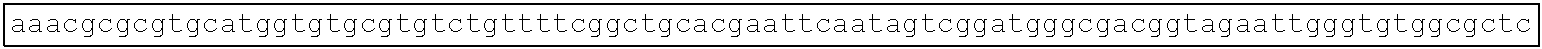

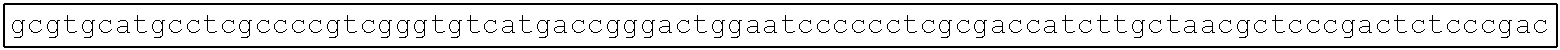

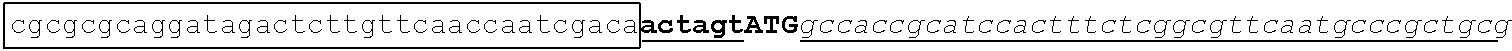

gcgacctgcgtcgctcggcgggctccgggccccggcgcccagcgaggcccctccccgtgcgcgggcgcgccatccccccccgca
tcatcgtggtgtcctcctcctcctccaaggtgaaccccctgaagaccgaggccgtggtgtcctccggcctggccgaccgcctgcg
cctgggctccctgaccgaggacggcctgtcctacaaggagaagttcatcgtgcgctgctacgaggtgggcatcaacaagacc
gccaccgtggagaccatcgccaacctgctgcaggaggtgggctgcaaccacgcccagtccgtgggctactccaccgccggctt
ctccaccacccccaccatgcgcaagctgcgcctgatctgggtgaccgcccgcatgcacatcgagatctacaagtaccccgcctg
gtccgacgtggtggagatcgagtcctggggccagggcgagggcaagatcggcacccgccgcgactggatcctgcgcgacta
cgccaccggccaggtgatcggccgcgccacctccaagtgggtgatgatgaaccaggacacccgccgcctgcagaaggtgga
cgtggacgtgcgcgacgagtacctggtgcactgcccccgcgagctgcgcctggccttccccgaggagaacaactcctccctga
agaagatctccaagctggaggacccctcccagtactccaagctgggcctggtgccccgccgcgccgacctggacatgaacca
gcacgtgaacaacgtgacctacatcggctgggtgctggagtccatgccccaggagatcatcgacacccacgagctgcagacc
atcaccctggactaccgccgcgagtgccagcacgacgacgtggtggactccctgacctcccccgagccctccgaggacgccga
ggccgtgttcaaccacaacggcaccaacggctccgccaacgtgtccgccaacgaccacggctgccgcaacttcctgcacctgct
gcgcctgtccggcaacggcctggagatcaaccgcggccgcaccgagtggcgcaagaagcccacccgcATGGACTACAA

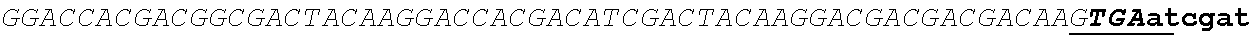

ggagcgacgagtgtgcgtgcggggctggcgggagtgggacgccctcctcgctcctctctgttctgaacggaacaatcggccacccc
gcgctacgcgccacgcatcgagcaacgaagaaaaccccccgatgataggttgcggtggctgccgggatatagatccggccgcaca
tcaaagggcccctccgccagagaagaagctcctttcccagcagactccttctgctgccaaaacacttctctgtccacagcaacacca
aaggatgaacagatcaacttgcgtctccgcgtagcttcctcggctagcgtgcttgcaacaggtccctgcactattatcttcctgctttc
ctctgaattatgcggcaggcgagcgctcgctctggcgagcgctccttcgcgccgccctcgctgatcgagtgtacagtcaatgaatggt
gagctcctcactcagcgcgcctgcgcggggatgcggaacgccgccgccgccttgtcttttgcacgcgcgactccgtcgcttcgcggg
tggcacccccattgaaaaaaacctcaattctgtttgtggaagacacggtgtacccccaaccacccacctgcacctctattattggta
ttattgacgcgggagcgggcgttgtactctacaacgtagcgtctctggttttcagctggctcccaccattgtaaattcttgctaaaat
agtgcgtggttatgtgagaggtatggtgtaacagggcgtcagtcatgttggttttcgtgctgatctcgggcacaaggcgtcgtcgac
gtgacgtgcccgtgatgagagcaataccgcgctcaaagccgacgcatggcctttactccgcactccaaacgactgtcgctcgtatt
tttcggatatctattttttaagagcgagcacagcgccgggcatgggcctgaaaggcctcgcggccgtgctcgtggtgggggccgcg
agcgcgtggggcatcgcggcagtgcaccaggcgcagacggaggaacgcatggtgagtgcgcatcacaagatgcatgtcttgttg
tctgtactataatgctagagcatcaccaggggcttagtcatcgcacctgctttggtcattacagaaattgcacaagggcgtcctccg
ggatgaggagatgtaccagctcaagctggagcggcttcgagccaagcaggagcgcggcgcatgacgacctacccacatgcgaa
gagc

The sequence of the pSZ6377 transforming DNA is provided below. Relevant restriction sites in the construct are indicated in lowercase, bold and underlined text, and are 5′-3′ BspQI, KpnI, XbaI, SnaBI, BamHI, AvrII, SpeI, AscI, ClaI, SacI and BspQ respectively. BspQI sites delimit the 5′ and 3′ ends of the transforming DNA. Proceeding in the 5′ to 3′ direction, bold, lowercase sequences represent FAD2-1 5′ genomic DNA that permits targeted integration at the FAD2-1 locus via homologous recombination. The P. moriformis LDH1 promoter driving the expression of the Arabidopsis thaliana THIC gene is indicated by boxed text. The initiator ATG and terminator TGA for AtTHIC are indicated by uppercase, bold italics while the coding region is indicated in lowercase italics. The P. moriformis HSP90 3′ UTR is indicated by lowercase underlined text. A spacer sequence is represented by lowercase text. The P. moriformis SAD2-1 promoter, indicated by boxed italicized text, is utilized to drive the expression of the G. mangostana FATA1 gene. The initiator ATG of the sequence encoding the C. protothecoides SAD1 transit peptide (CpSAD1tp) is indicated by uppercase, bold italics, and the remainder of the CpSAD1tp sequence located between the ATG and the AscI site is indicated with lowercase, underlined italics. The GarmFATA1(G108A) coding region is indicated by lowercase italics. A sequence encoding a 3×FLAG tag fused to the C-terminus of GarmFATA1(G108A) is represented by uppercase italics, and the TGA terminator codon is indicated with uppercase, bold italics. The P. moriformis SAD2-1 3′ UTR is indicated by lowercase underlined text. The FAD2-1 3′ genomic region is indicated by bold, lowercase text.

Nucleotide sequence of transforming DNA contained in pSZ6377
SEQ ID NO:130
gctcttcgcgaaggtcattttccagaacaacgaccatggcttgtcttagcgatcgctcgaatgactgctagtgagtcgtacgctcga
cccagtcgctcgcaggagaacgcggcaactgccgagcttcggcttgccagtcgtgactcgtatgtgatcaggaatcattggcattg
gtagcattataattcggcttccgcgctgtttatgggcatggcaatgtctcatgcagtcgaccttagtcaaccaattctgggtggccag
ctccgggcgaccgggctccgtgtcgccgggcaccacctcctgccatgagtaacagggccgccctctcctcccgacgttggccaact
gaataccgtgtcttggggccctacatgatgggctgcctagtcgggcgggacgcgcaactgcccgcgcaatctgggacgtggtctga
atcctccaggcgggtttccccgagaaagaaagggtgccgatttcaaagcagagccatgtgccgggccctgtggcctgtgttggcgc
ctatgtagtcaccccccctcacccaattgtcgccagtttgcgcaatccataaactcaaaactgcagcttctgagctgcgctgttcaa

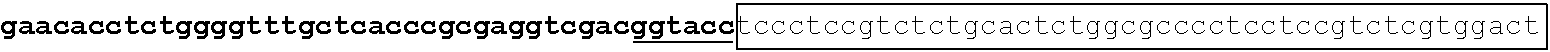

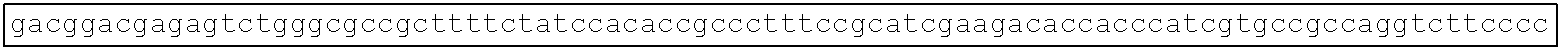

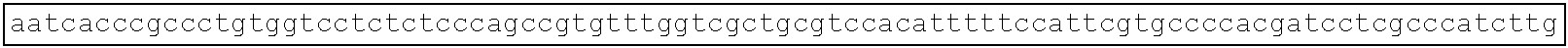

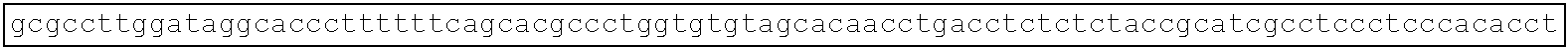

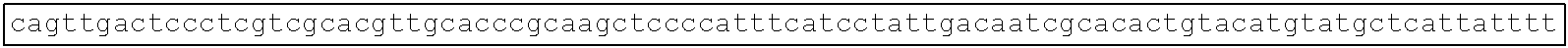

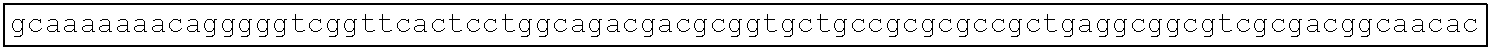

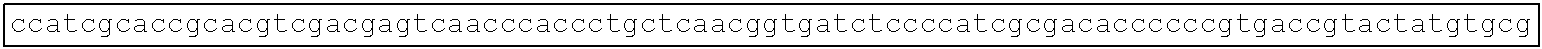

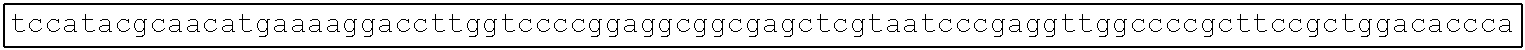

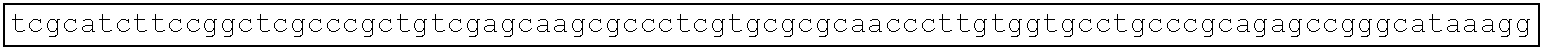

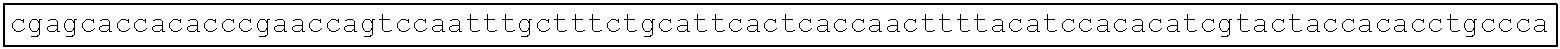

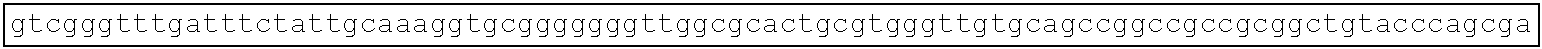

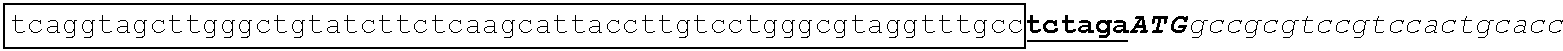

ctgatgtccgtggtctgcaacaacaagaaccactccgcccgccccaagctgcccaactcctccctgctgcccggcttcgacgtgg
tggtccaggccgcggccacccgcttcaagaaggagacgacgaccacccgcgccacgctgacgttcgacccccccacgaccaa
ctccgagcgcgccaagcagcgcaagcacaccatcgacccctcctcccccgacttccagcccatcccctccttcgaggagtgcttc
cccaagtccacgaaggagcacaaggaggtggtgcacgaggagtccggccacgtcctgaaggtgcccttccgccgcgtgcac
ctgtccggcggcgagcccgccttcgacaactacgacacgtccggcccccagaacgtcaacgcccacatcggcctggcgaagct
gcgcaaggagtggatcgaccgccgcgagaagctgggcacgccccgctacacgcagatgtactacgcgaagcagggcatcat
cacggaggagatgctgtactgcgcgacgcgcgagaagctggaccccgagttcgtccgctccgaggtcgcgcggggccgcgc
catcatcccctccaacaagaagcacctggagctggagcccatgatcgtgggccgcaagttcctggtgaaggtgaacgcgaac
atcggcaactccgccgtggcctcctccatcgaggaggaggtctacaaggtgcagtgggccaccatgtggggcgccgacacca
tcatggacctgtccacgggccgccacatccacgagacgcgcgagtggatcctgcgcaactccgcggtccccgtgggcaccgtc
cccatctaccaggcgctggagaaggtggacggcatcgcggagaacctgaactgggaggtgttccgcgagacgctgatcgag
caggccgagcagggcgtggactacttcacgatccacgcgggcgtgctgctgcgctacatccccctgaccgccaagcgcctgac
gggcatcgtgtcccgcggcggctccatccacgcgaagtggtgcctggcctaccacaaggagaacttcgcctacgagcactggg
acgacatcctggacatctgcaaccagtacgacgtcgccctgtccatcggcgacggcctgcgccccggctccatctacgacgcca
acgacacggcccagttcgccgagctgctgacccagggcgagctgacgcgccgcgcgtgggagaaggacgtgcaggtgatg
aacgagggccccggccacgtgcccatgcacaagatccccgagaacatgcagaagcagctggagtggtgcaacgaggcgcc
cttctacaccctgggccccctgacgaccgacatcgcgcccggctacgaccacatcacctccgccatcggcgcggccaacatcgg
cgccctgggcaccgccctgctgtgctacgtgacgcccaaggagcacctgggcctgcccaaccgcgacgacgtgaaggcgggc
gtcatcgcctacaagatcgccgcccacgcggccgacctggccaagcagcacccccacgcccaggcgtgggacgacgcgctgt
ccaaggcgcgcttcgagttccgctggatggaccagttcgcgctgtccctggaccccatgacggcgatgtccttccacgacgaga
cgctgcccgcggacggcgcgaaggtcgcccacttctgctccatgtgcggccccaagttctgctccatgaagatcacggaggac
atccgcaagtacgccgaggagaacggctacggctccgccgaggaggccatccgccagggcatggacgccatgtccgagga
gttcaacatcgccaagaagacgatctccggcgagcagcacggcgaggtcggcggcgagatctacctgcccgagtcctacgtc

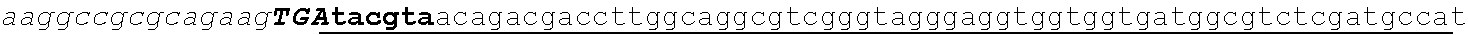

cgcacgcatccaacgaccgtatacgcatcgtccaatgaccgtcggtgtcctctctgcctccgttttgtgagatgtctcaggcttggtgc
atcctcgggtggccagccacgttgcgcgtcgtgctgcttgcctctcttgcgcctctgtggtactggaaaatatcatcgaggcccgttttt
ttgctcccatttcctttccgctacatcttgaaagcaaacgacaaacgaagcagcaagcaaagagcacgaggacggtgaacaagtct
gtcacctgtatacatctatttccccgcgggtgcacctactctctctcctgccccggcagagtcagctgccttacgtgacggatcccgcg
tctcgaacagagcgcgcagaggaacgctgaaggtctcgcctctgtcgcacctcagcgcggcatacaccacaataaccacctgacga
atgcgcttggttcttcgtccattagcgaagcgtccggttcacacacgtgccacgttggcgaggtggcaggtgacaatgatcggtgga

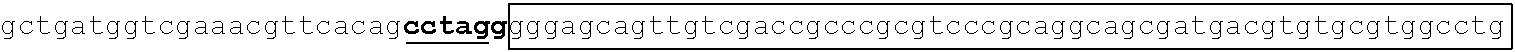

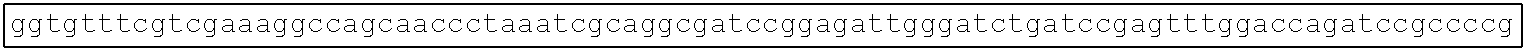

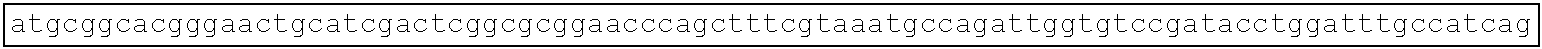

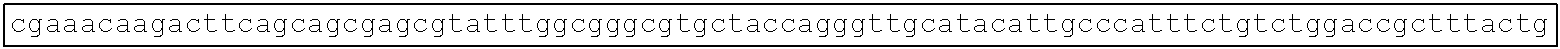

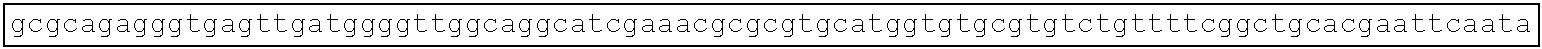

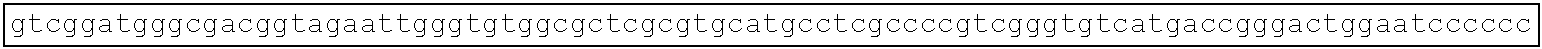

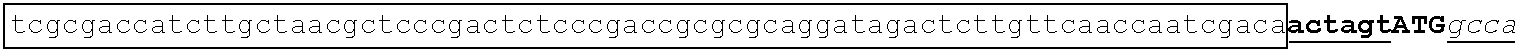

ccgcatccactttctcggcgttcaatgcccgctgcggcgacctgcgtcgctcggcgggctccgggccccggcgcccagcgaggc
ccctccccgtgcgcgggcgcgccatccccccccgcatcatcgtggtgtcctcctcctcctccaaggtgaaccccctgaagaccgag
gccgtggtgtcctccggcctggccgaccgcctgcgcctgggctccctgaccgaggacggcctgtcctacaaggagaagttcatc
gtgcgctgctacgaggtgggcatcaacaagaccgccaccgtggagaccatcgccaacctgctgcaggaggtgggctgcaac
cacgcccagtccgtgggctactccaccgccggcttctccaccacccccaccatgcgcaagctgcgcctgatctgggtgaccgccc
gcatgcacatcgagatctacaagtaccccgcctggtccgacgtggtggagatcgagtcctggggccagggcgagggcaaga
tcggcacccgccgcgactggatcctgcgcgactacgccaccggccaggtgatcggccgcgccacctccaagtgggtgatgatg
aaccaggacacccgccgcctgcagaaggtggacgtggacgtgcgcgacgagtacctggtgcactgcccccgcgagctgcgcc
tggccttccccgaggagaacaactcctccctgaagaagatctccaagctggaggacccctcccagtactccaagctgggcctg
gtgccccgccgcgccgacctggacatgaaccagcacgtgaacaacgtgacctacatcggctgggtgctggagtccatgcccca
ggagatcatcgacacccacgagctgcagaccatcaccctggactaccgccgcgagtgccagcacgacgacgtggtggactcc
ctgacctcccccgagccctccgaggacgccgaggccgtgttcaaccacaacggcaccaacggctccgccaacgtgtccgccaa
cgaccacggctgccgcaacttcctgcacctgctgcgcctgtccggcaacggcctggagatcaaccgcggccgcaccgagtggc
gcaagaagcccacccgcATGGACTACAAGGACCACGACGGCGACTACAAGGACCACGACATCGACTACA

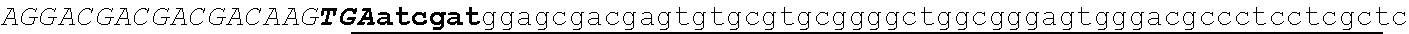

ctctctgttctgaacggaacaatcggccaccccgcgctacgcgccacgcatcgagcaacgaagaaaaccccccgatgataggttgc
ggtggctgccgggatatagatccggccgcacatcaaagggcccctccgccagagaagaagctcctttcccagcagactccttctgct
gccaaaacacttctctgtccacagcaacaccaaaggatgaacagatcaacttgcgtctccgcgtagcttcctcggctagcgtgcttg
caacaggtccctgcactattatcttcctgctttcctctgaattatgcggcaggcgagcgctcgctctggcgagcgctccttcgcgccgc
cctcgctgatcgagtgtacagtcaatgaatggtgagctcctcactcagcgcgcctgcgcggggatgcggaacgccgccgccgcctt
gtcttttgcacgcgcgactccgtcgcttcgcgggtggcacccccattgaaaaaaacctcaattctgtttgtggaagacacggtgtac
ccccaaccacccacctgcacctctattattggtattattgacgcgggagcgggcgttgtactctacaacgtagcgtctctggttttca
gctggctcccaccattgtaaattcttgctaaaatagtgcgtggttatgtgagaggtatggtgtaacagggcgtcagtcatgttggtt
ttcgtgctgatctcgggcacaaggcgtcgtcgacgtgacgtgcccgtgatgagagcaataccgcgctcaaagccgacgcatggcc
tttactccgcactccaaacgactgtcgctcgtatttttcggatatctattttttaagagcgagcacagcgccgggcatgggcctgaa
aggcctcgcggccgtgctcgtggtgggggccgcgagcgcgtggggcatcgcggcagtgcaccaggcgcagacggaggaacgcat
ggtgagtgcgcatcacaagatgcatgtcttgttgtctgtactataatgctagagcatcaccaggggcttagtcatcgcacctgcttt
ggtcattacagaaattgcacaagggcgtcctccgggatgaggagatgtaccagctcaagctggagcggcttcgagccaagcagg
agcgcggcgcatgacgacctacccacatgcgaagagc

Constructs pSZ6383, pSZ6384 and pSZ6377 were transformed into S8813. Primary transformants were clonally purified and screened under standard lipid production conditions at pH 5. Integration of pSZ6383 or pSZ6384 at the FAD2-1 locus was verified by DNA blot analysis. The fatty acid profiles, sn-2 profiles and lipid titers of lead strains were assayed in 50-mL shake flasks (Table 23). FAD2-1 ablation reduced C18:2 to <1% in most strains. Expression of a second copy of GarmFATA1(G108A) and TcDGAT1 (S8990, S8992, S8998 & S8999), or TcDGAT2 (S8994, S9000 & S9047) elevated C18:0 to >56%. The D5393-28 strain, expressing a second copy of GarmFATA1(G108A) without either of the cocoa DGAT genes (pSZ6377) had a similar fatty acid profile, but lower lipid titer. As shown in Table 23, as compared to strain S8813, for strains expressing either TcDGAT1 or TcDGAT2, C16:0 increased from 3.2% to 3.7%-4.0%, C18:0 increased from 45.8% to about 56%, C18:2 decreased from 1.4% to about 1.0%.

TABLE 23
Fatty acid profiles of FAD2-1 ablation strains.

| Strain | S8813 | D5393-28 | S8990 | S8992 | S8998 | S8999 | S8994 | S9000 | S9047 |
|---|---|---|---|---|---|---|---|---|---|
| C12:0 | 0.1 | 0.2 | 0.2 | 0.2 | 0.1 | 0.2 | 0.1 | 0.1 | 0.2 |
| C14:0 | 0.4 | 0.5 | 0.5 | 0.5 | 0.5 | 0.5 | 0.5 | 0.5 | 0.5 |
| C16:0 | 3.2 | 3.8 | 3.7 | 3.8 | 3.9 | 4.0 | 3.7 | 3.8 | 3.5 |
| C16:1 cis-7 | 0.1 | 0.1 | 0.1 | 0.1 | 0.1 | 0.1 | 0.1 | 0.1 | 0.1 |
| C16:1 cis-9 | 0.0 | 0.1 | 0.1 | 0.1 | 0.1 | 0.1 | 0.1 | 0.1 | 0.1 |
| C17:0 | 0.1 | 0.2 | 0.2 | 0.1 | 0.2 | 0.1 | 0.2 | 0.2 | 0.2 |
| C18:0 | 45.8 | 56.0 | 56.6 | 56.0 | 56.2 | 56.0 | 56.3 | 56.4 | 56.5 |
| C18:1 | 45.9 | 35.8 | 35.4 | 35.9 | 35.7 | 35.5 | 35.9 | 35.7 | 35.9 |
| C18:2 | 1.4 | 1.0 | 0.9 | 1.0 | 0.9 | 1.1 | 0.9 | 0.9 | 0.8 |
| C18:3 α | 0.3 | 0.3 | 0.3 | 0.2 | 0.3 | 0.2 | 0.2 | 0.3 | 0.3 |
| C20:0 | 2.0 | 1.6 | 1.6 | 1.5 | 1.6 | 1.5 | 1.5 | 1.5 | 1.5 |
| C22:0 | 0.2 | 0.2 | 0.2 | 0.2 | 0.2 | 0.2 | 0.2 | 0.2 | 0.2 |
| C24:0 | 0.1 | 0.1 | 0.1 | 0.1 | 0.1 | 0.1 | 0.1 | 0.1 | 0.1 |
| saturates | 52.1 | 62.6 | 63.1 | 62.6 | 62.9 | 62.8 | 62.8 | 62.9 | 62.7 |

Liquid chromatography and mass spectrometry were used to analyze the TAG composition of final strains. The strains accumulated 68-71% SOS, with trisaturates ranging from 2.5-2.8%. The D5393-28 strain, expressing a second copy of GarmFATA1(G108A) without either of the cocoa DGAT genes had similar SOS content but slightly higher trisaturates. The TAG composition of a typical Shea stearin and a sample of Kokum butter are shown for comparison

TABLE 24
LC/MS TAG profiles of FAD2-1 ablation strains.

| | | | | | | | | | Shea | Kokum |
|---|---|---|---|---|---|---|---|---|---|---|
| Strain | D5393-28 | S8990 | S8992 | S8998 | S8999 | S8994 | S9000 | S9047 | stearin | butter |
| OOL | | | | | | | | | 0.4 | |
| LLS | | | | | | | | | 0.2 | |
| POL | | | | | | | | | 0.3 | |
| OOO | | | | | | | | | 1.3 | 1.7 |
| SOL | | | | | | | | | 1.0 | 0.4 |
| LaOS + MOP | 0.2 | 0.3 | 0.3 | 0.2 | 0.3 | 0.3 | 0.4 | 0.2 | | |
| OOP | 0.5 | 0.2 | 0.3 | 0.2 | 0.2 | 0.4 | 0.3 | 0.2 | 0.8 | 0.7 |
| PLS (+SLnS) | 0.6 | 0.7 | 0.7 | 0.7 | 0.7 | 0.6 | 0.6 | 0.4 | 0.6 | 0.3 |
| POP (+MOS) | 1.1 | 1.0 | 1.0 | 1.1 | 1.1 | 1.0 | 1.2 | 0.8 | 0.7 | 0.4 |
| OOS | 10.5 | 10.3 | 11.3 | 11.0 | 11.0 | 10.9 | 10.1 | 10.6 | 6.4 | 11.8 |
| SLS (+PLA) | 1.9 | 1.7 | 2.0 | 1.6 | 2.1 | 1.8 | 1.9 | 1.5 | 5.5 | 1.4 |
| POS | 8.4 | 8.5 | 8.4 | 8.7 | 8.9 | 8.4 | 10.0 | 7.7 | 6.3 | 4.8 |
| MaOS | 0.3 | | | | | | | | | |
| SOG | 0.4 | 0.5 | 0.5 | 0.6 | 0.3 | 0.5 | 0.4 | 0.5 | | |
| OOA | 0.5 | 0.3 | 0.4 | 0.4 | 0.4 | 0.4 | 0.4 | 0.4 | 0.2 | 0.2 |
| SOS (+POA) | 68.4 | 69.7 | 68.7 | 69.1 | 68.3 | 69.4 | 68.0 | 71.4 | 69.7 | 76.6 |
| SSP (+MSA) | 0.5 | 0.5 | 0.5 | 0.4 | 0.5 | 0.5 | 0.5 | 0.4 | 0.2 | |
| SOA + POB | 3.9 | 3.8 | 3.5 | 3.6 | 3.4 | 3.5 | 3.5 | 3.4 | 4.0 | 1.0 |
| SSS (+PSA) | 2.6 | 2.3 | 2.2 | 2.1 | 2.3 | 2.2 | 2.3 | 2.1 | 2.0 | 0.5 |
| SOB + LgOP + AOA | 0.4 | 0.2 | 0.2 | 0.3 | 0.3 | 0.3 | 0.3 | 0.3 | 0.4 | |
| SSA (+PBS) | | | | | | | | | 0.2 | |
| SOLg (+POHx) | | | | | | | | | 0.3 | |
| SUM (area %) | 99.8 | 99.9 | 99.8 | 99.9 | 99.8 | 99.9 | 100.0 | 99.9 | 100.0 | 100.0 |
| Sat-Sat-Sat | 3.1 | 2.8 | 2.7 | 2.5 | 2.7 | 2.7 | 2.8 | 2.5 | 2.4 | 0.5 |
| Sat-U-Sat | 84.9 | 85.9 | 84.7 | 85.3 | 85.1 | 85.0 | 86.0 | 85.8 | 87.5 | 84.7 |
| Sat-O-Sat | 82.4 | 83.5 | 82.0 | 82.9 | 82.3 | 82.6 | 83.4 | 83.9 | 81.4 | 83.1 |
| Sat-L-Sat | 2.5 | 2.4 | 2.6 | 2.3 | 2.8 | 2.4 | 2.6 | 1.9 | 6.1 | 1.6 |
| U-U-U/Sat | 11.8 | 11.3 | 12.4 | 12.2 | 12.0 | 12.2 | 11.3 | 11.7 | 10.6 | 14.8 |

La = laurate (C12:0), M = myristate (C14:0), P = palmitate (C16:0), Ma = margarate (C17:0), S = stearate (C18:0), O = oleate (C18:1), L = linoleate (C18:2), Ln = α-linolenate (C18:3 α), A = arachidate (C20:0), G = (C20:1), B = behenate (C22:0), Lg = lignocerate (C24:0), Hx = hexacosanoate (C26:0). Sat = saturated, U = unsaturated

### Example 8 Variant Brassica napus Thioeserase

In this example, we demonstrate the modification of the enzyme specificity of a FATA thioesterase originally isolated from Brassica napus (BnOTE, accession CAA52070), by site directed mutagenesis targeting two amino acids positions D124 and D209).

To determine the impact of each amino acid substitution on the enzyme specificity of the BnOTE, the wild-type and the mutant BnOTE genes were cloned into a vector enabling expression and expressed in P. moriformis strain S8588. Strain S8588 is a strain in which the endogenous FATA1 allele has been disrupted and expresses a Prototheca moriformis KASII gene and sucrose invertase. Recombinant strains with FATA1 disruption and co-expression of P. moriformis KASII and invertase were previously disclosed in co-owned applications WO2012/106560 and WO2013/15898, herein incorporated by reference.

Strains that express wild type or mutant BnOTE enzymes, constructs pSZ6315, pSZ6316, pSZ6317, or pSZ6318 were expressed in S8588. In these constructs, the Saccharomyces carlsbergensis MEL1 gene (Accession no: AAA34770) was utilized as the selectable marker to introduce the wild-type and mutant BnOTE genes into the FAD2-2 locus of P. moriformis strain S8588 by homologous recombination using previously described transformation methods (biolistics). The constructs that have been expressed in S8588 are listed in Table 25.

TABLE 25
DNA lot# and plasmid ID of DNA constructs that expressing wild-type and mutant BnOTE genes

| DNA | Solazynne | |
|---|---|---|
| Lot# | Plasmid | Construct |
| D5309 | pSZ6315 | FAD2-2::PmHXT1-ScarMEL1-PmPGK:PmSAD2-2 V3-CpSADtp-BnOTE-PmSAD2-1 |
| | | utr::FAD2-2 |
| D5310 | pSZ6316 | FAD2-2::PmHXT1-ScarMEL1-PmPGK:PmSAD2-2 V3-CpSADtp-BnOTE(D124A)-PmSAD2-1 |
| | | utr::FAD2-2 |
| D5311 | pSZ6317 | FAD2-2::PmHXT1-ScarMEL1-PmPGK:PmSAD2-2 V3-CpSADtp-BnOTE(D209A)-PmSAD2-1 |
| | | utr::FAD2-2 |
| D5312 | pSZ6318 | FAD2-2::PmHXT1-ScarMEL1-PmPGK:PmSAD2-2 V3-CpSADtp-BnOTE(D124A, D209A)- |
| | | PmSAD2-1 utr::FAD2-2 |

pSZ6315

The construct psZ6315 can be written as FAD2-2::PmHXT1-ScarMEL1-PmPGK:PmSAD2-2 V3-CpSADtp-BnOTE-PmSAD2-1 utr::FAD2-2. The sequence of the pSZ6315 transforming DNA is provided below. Relevant restriction sites in pSZ6315 are indicated in lowercase, bold and underlining and are 5′-3′ SgrAI, Kpn I, SnaBI, AvrII, SpeI, AscI, ClaI, Sac I, SbfI, respectively. SgrAI and SbfI sites delimit the 5′ and 3′ ends of the transforming DNA. Bold, lowercase sequences represent FAD2-2 genomic DNA that permit targeted integration at FAD2-2 locus via homologous recombination. Proceeding in the 5′ to 3′ direction, the P. moriformis HXT1 promoter driving the expression of the Saccharomyces carlsbergensis MEL1 gene is indicated by boxed text. The initiator ATG and terminator TGA for MEL1 gene are indicated by uppercase, bold italics while the coding region is indicated in lowercase italics. The P. moriformis PGK 3′ UTR is indicated by lowercase underlined text followed by the P. moriformis SAD2-2 V3 promoter, indicated by boxed italics text. The Initiator ATG and terminator TGA codons of the wild-type BnOTE are indicated by uppercase, bold italics, while the remainder of the coding region is indicated by bold italics in lower case. The three-nucleotide codon corresponding to the target amino acids, D124 and D209, are in lower case, italicized, bolded and wave underlined. The P. moriformis SAD2-1 3′UTR is again indicated by lowercase underlined text followed by the FAD2-2 genomic region indicated by bold, lowercase text.

Nucleotide sequence of transforming DNA contained in pSZ6315
SEQ ID NO: 131
caccggcgcgctgcttcgcgtgccgggtgcagcaatcagatccaagtctgacgacttgcgcgcacgcgccggatccttcaattccaaagtgtcg
tccgcgtgcgcttcttcgccttcgtcctcttgaacatccagcgacgcaagcgcagggcgctgggcggctggcgtcccgaaccggcctcggcgcac
gcggctgaaattgccgatgtcggcaatgtagtgccgctccgcccacctctcaattaagtttttcagcgcgtggttgggaatgatctgcgctcatg
gggcgaaagaaggggttcagaggtgctttattgttactcgactgggcgtaccagcattcgtgcatgactgattatacatacaaaagtacagctc
gcttcaatgccctgcgattcctactcccgagcgagcactcctctcaccgtcgggttgcttcccacgaccacgccggtaagagggtctgtggcctc
gcgcccctcgcgagcgcatattccagccacgtctgtatgattttgcgctcatacgtctggcccgtcgaccccaaaatgacgggatcctgcataa
tatcgcccgaaatgggatccaggcattcgtcaggaggcgtcagccccgcgggagatgccggtcccgccgcattggaaaggtgtagagggggt

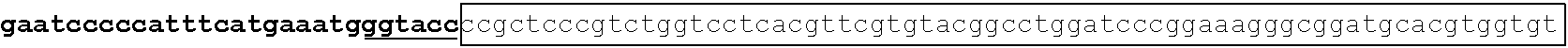

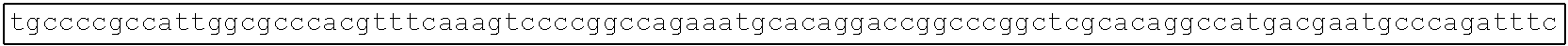

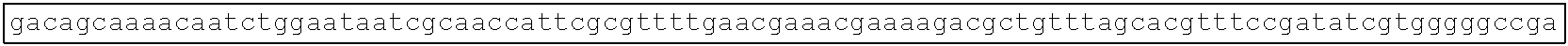

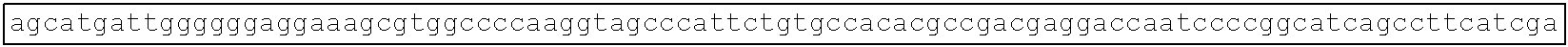

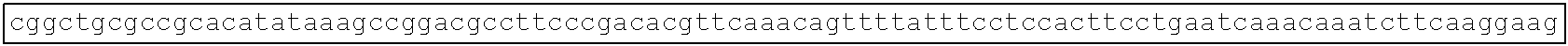

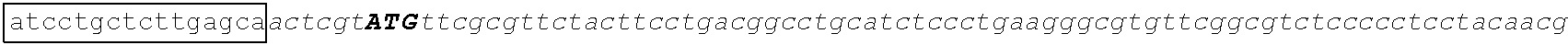

gcctgggcctgacgccccagatgggctgggacaactggaacacgttcgcctgcgacgtctccgagcagctgctgctggacacggccgacc
gcatctccgacctgggcctgaaggacatgggctacaagtacatcatcctggacgactgctggtcctccggccgcgactccgacggcttcctg
gtcgccgacgagcagaagttccccaacggcatgggccacgtcgccgaccacctgcacaacaactccttcctgttcggcatgtactcctccgc
gggcgagtacacgtgcgccggctaccccggctccctgggccgcgaggaggaggacgcccagttcttcgcgaacaaccgcgtggactacct
gaagtacgacaactgctacaacaagggccagttcggcacgcccgagatctcctaccaccgctacaaggccatgtccgacgccctgaacaa
gacgggccgccccatcttctactccctgtgcaactggggccaggacctgaccttctactggggctccggcatcgcgaactcctggcgcatgtc
cggcgacgtcacggcggagttcacgcgccccgactcccgctgcccctgcgacggcgacgagtacgactgcaagtacgccggcttccactgc
tccatcatgaacatcctgaacaaggccgcccccatgggccagaacgcgggcgtcggcggctggaacgacctggacaacctggaggtcgg
cgtcggcaacctgacggacgacgaggagaaggcgcacttctccatgtgggccatggtgaagtcccccctgatcatcggcgcgaacgtga
acaacctgaaggcctcctcctactccatctactcccaggcgtccgtcatcgccatcaaccaggactccaacggcatccccgccacgcgcgtct
ggcgctactacgtgtccgacacggacgagtacggccagggcgagatccagatgtggtccggccccctggacaacggcgaccaggtcgtg
gcgctgctgaacggcggctccgtgtcccgccccatgaacacgaccctggaggagatcttcttcgactccaacctgggctccaagaagctga
cctccacctgggacatctacgacctgtgggcgaaccgcgtcgacaactccacggcgtccgccatcctgggccgcaacaagaccgccaccg
gcatcctgtacaacgccaccgagcagtcctacaaggacggcctgtccaagaacgacacccgcctgttcggccagaagatcggctccctgtc

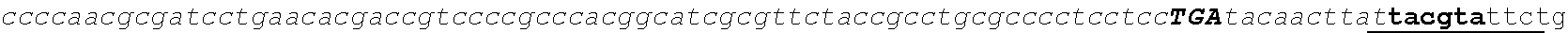

accggcgctgatgtggcgcggacgccgtcgtactctttcagactttactcttgaggaattgaacctttctcgcttgctggcatgtaaacattggcgc
aattaattgtgtgatgaagaaagggtggcacaagatggatcgcgaatgtacgagatcgacaacgatggtgattgttatgaggggccaaacctg
gctcaatcttgtcgcatgtccggcgcaatgtgatccagcggcgtgactctcgcaacctggtagtgtgtgcgcaccgggtcgctttgattaaaactg
atcgcattgccatcccgtcaactcacaagcctactctagctcccattgcgcactcgggcgcccggctcgatcaatgttctgagcggagggcgaag
cgtcaggaaatcgtctcggcagctggaagcgcatggaatgcggagcggagatcgaatcaggatcccgcgtctcgaacagagcgcgcagagga
acgctgaaggtctcgcctctgtcgcacctcagcgcggcatacaccacaataaccacctgacgaatgcgcttggttcttcgtccattagcgaagcgt

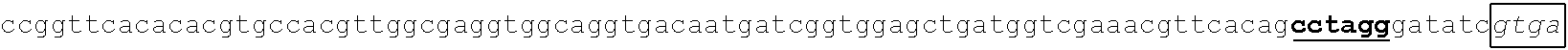

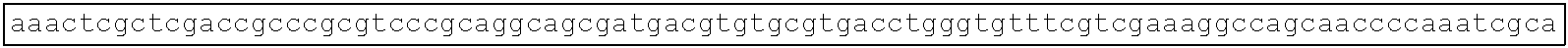

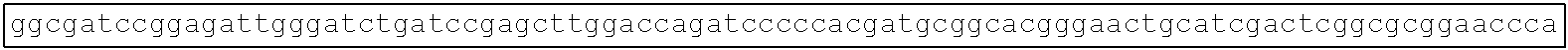

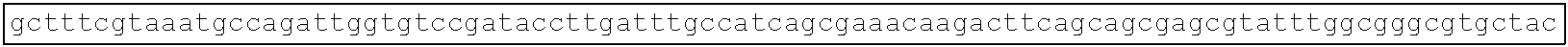

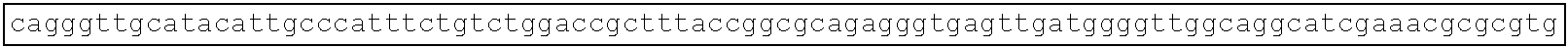

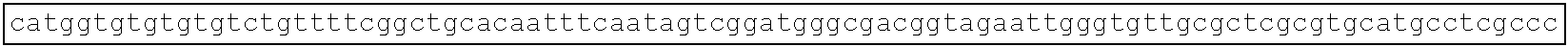

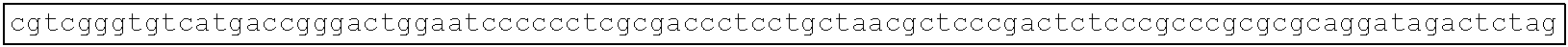

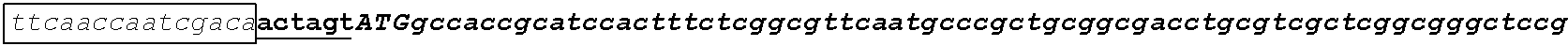

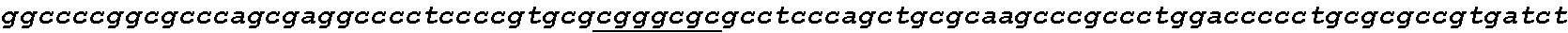

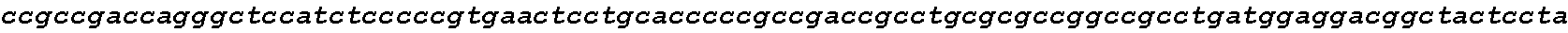

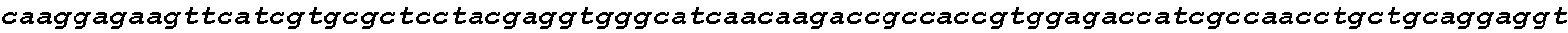

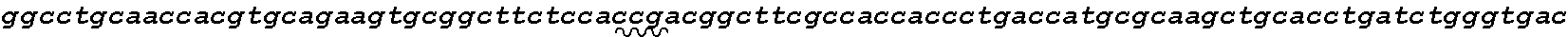

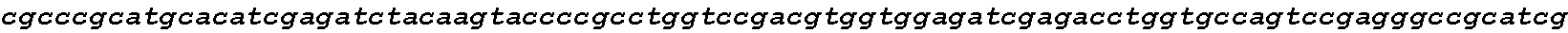

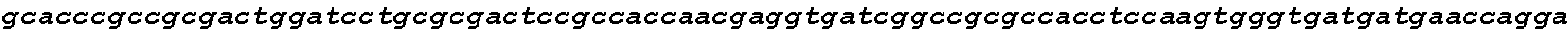

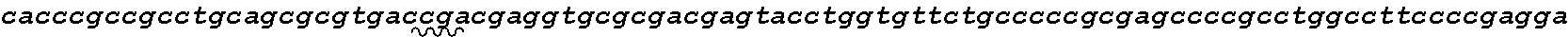

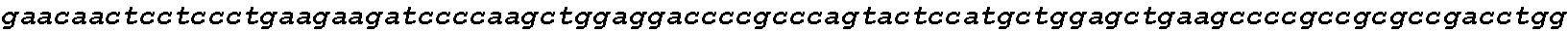

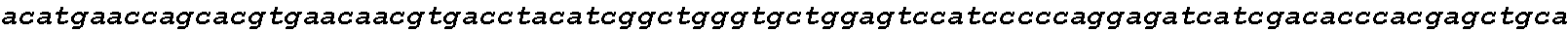

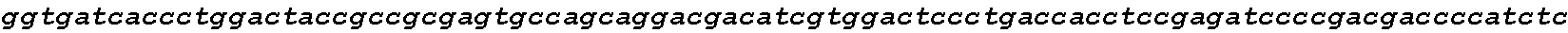

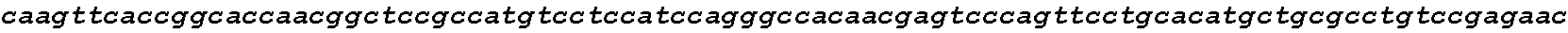

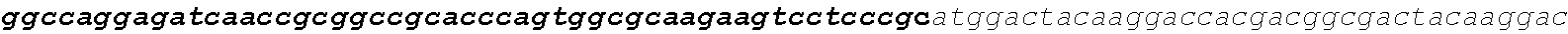

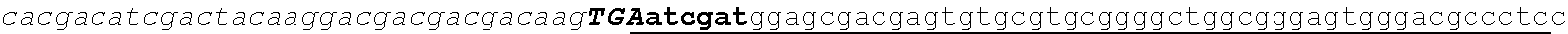

tcgctcctctctgttctgaacggaacaatcggccaccccgcgctacgcgccacgcatcgagcaacgaagaaaaccccccgatgataggttgcgg
tggctgccgggatatagatccggccgcacatcaaagggcccctccgccagagaagaagctcctttcccagcagactccttctgctgccaaaaca
cttctctgtccacagcaacaccaaaggatgaacagatcaacttgcgtctccgcgtagcttcctcggctagcgtgcttgcaacaggtccctgcacta
ttatcttcctgctttcctctgaattatgcggcaggcgagcgctcgctctggcgagcgctccttcgcgccgccctcgctgatcgagtgtacagtcaat
gaatggtgagctccgcgcctgcgcgaggacgcagaacaacgctgccgccgtgtcttttgcacgcgcgactccggcgcttcgctggtggcacccc
cataaagaaaccctcaattctgtttgtggaagacacggtgtacccccacccacccacctgcacctctattattggtattattgacgcgggagtgg
gcgttgtaccctacaacgtagcttctctagttttcagctggctcccaccattgtaaattcatgctagaatagtgcgtggttatgtgagaggtatag
tgtgtctgagcagacggggcgggatgcatgtcgtggtggtgatctttggctcaaggcgtcgtcgacgtgacgtgcccgatcatgagagcaatac
cgcgctcaaagccgacgcatagcctttactccgcaatccaaacgactgtcgctcgtattifttggatatctattttaaagagcgagcacagcgcc
gggcatgggcctgaaaggcctcgcggccgtgctcgtggtgggggccgcgagcgcgtggggcatcgcggcagtgcaccaggcgcagacggag
gaacgcatggtgcgtgcgcaatataagatacatgtattgttgtcctgcagg
Nucleotide sequence of BnOTE (D124A) in pSZ6316
SEQ ID NO: 132

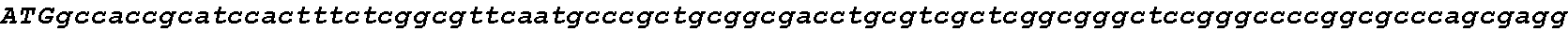

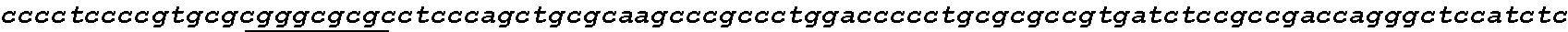

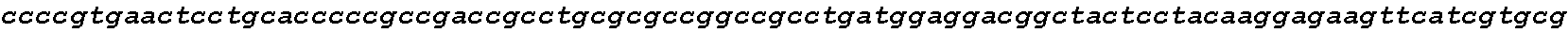

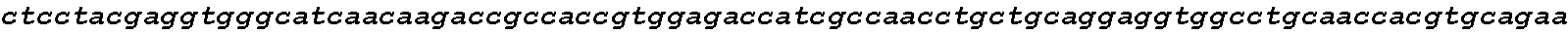

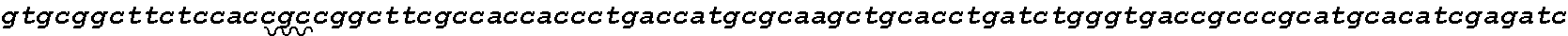

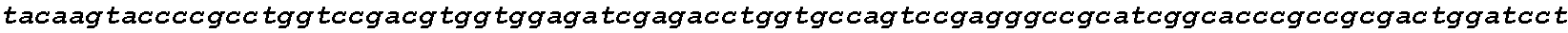

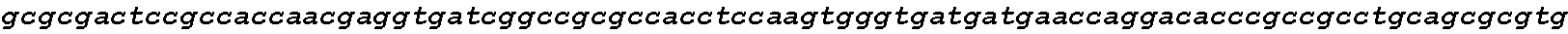

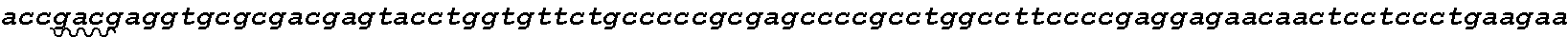

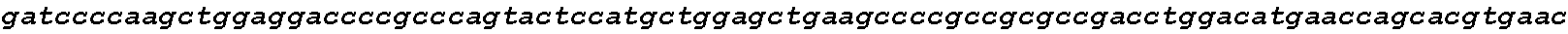

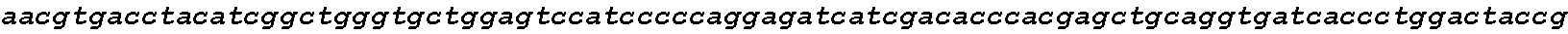

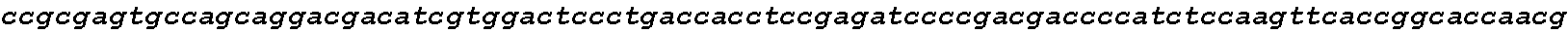

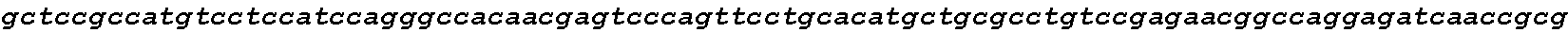

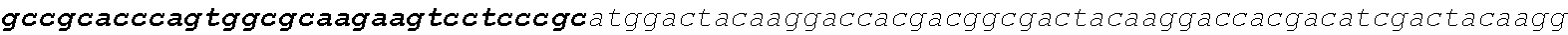

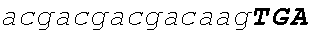

The sequence of the pSZ6317 transforming DNA is same as pSZ6315 except the D209A point mutation, the BnOTE D209A DNA sequence is provided below. The three-nucleotide codon corresponding to the target two amino acids, D124 and D209, are in lower case, italicized, bolded and wave underlined. pSZ6317 is written as FAD2-2::PmHXT1-ScarMEL1-PmPGK:PmSAD2-2 V3-CpSADtp-BnOTE (D209A)-PmSAD2-1 utr::FAD2-2

SEQ ID NO: 133 Nucleotide sequence of BnOTE (D209A) in pSZ6317:

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

atggactacaaggaccacgacggcgactacaaggaccacgacatcgactacaagg
acgacgacgacaag

[symbol]

The sequence of the pSZ6318 transforming DNA is same as pSZ6315 except two point mutations, D124A and D209A, the BnOTE (D124A, D209A) DNA sequence is provided below. The three-nucleotide codon corresponding to the target two amino acids, D124 and D209, are in lower case, italicized, bolded and wave underlined. pSZ6318 is written as FAD2-2::PmHXT1-ScarMEL1-PmPGK:PmSAD2-2 V3-CpSADtp-BnOTE (D124A, D209A)-PmSAD2-1 utr::FAD2-2

SEQ ID NO: 134 Nucleotide sequence of BnOTE (D124A, D209A) in pSZ6318

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

atggactacaaggaccacgacggcgactacaaggaccacgacatcgactacaagg
acgacgacgacaag

[symbol]

The DNA constructs containing the wild-type and mutant BnOTE genes were transformed into the parental strain S8588. Primary transformants were clonally purified and grown under standard lipid production conditions at pH5.0. The resulting profiles from representative clones arising from transformations with pSZ6315, pSZ6316, pSZ6317, and pSZ6318 into S8588 are shown in Table 26. The parental strain S8588 produces 5.4% C18:0, when transformed with the DNA cassette expressing wild-type BnOTE, the transgenic lines produce ˜11% C18:0. The BnOTE mutant (D124A) increased the amount of C18:0 by at least 2 fold compared to the wild-type protein. In contrast, the BnOTE D209A mutation appears to have no impact on the enzyme activity/specificity of the BnOTE thioesterase. Finally, expression of the BnOTE (D124A, D209A) resulted in very similar fatty acid profile to what we observed in the transformants from S8588 expressing BnOTE (D124A), again indicating that D209A has no significant impact on the enzyme activity.

TABLE 26
Fatty acid profiles in S8588 and derivative transgenic lines transformed with
wild-type and mutant BnOTE genes

| | | Fatty Acid Area % | | | |
|---|---|---|---|---|---|
| Transforming DNA | Sample ID | C16:0 | C18:0 | C18:1 | C18:2 |
| | pH5; S8588 (parental strain) | 3.00 | 5.43 | 81.75 | 6.47 |
| D5309, pSZ6315; | pH5; S8588, D5309-6; | 3.86 | 11.68 | 76.51 | 5.06 |
| wild-type BnOTE | pH5; S8588, D5309-2; | 3.50 | 11.00 | 77.80 | 4.95 |
| | pH5; S8588, D5309-9 ; | 3.51 | 10.72 | 78.03 | 5.00 |
| | pH5; S8588, D5309-10; | 3.55 | 10.69 | 78.06 | 4.96 |
| | pH5; S8588, D5309-11; | 3.61 | 10.69 | 78.05 | 4.95 |
| D5310, pSZ6316, | pH5; S8588, D5310-6; | 4.27 | 31.55 | 55.31 | 5.30 |
| BnOTE (D124A) | pH5; S8588, D5310-1; | 4.53 | 30.85 | 54.71 | 6.03 |
| | pH5; S8588, D5310-5; | 5.21 | 20.75 | 65.43 | 5.02 |
| | pH5; S8588, D5310-10; | 4.99 | 19.18 | 67.75 | 5.00 |
| | pH5; S8588, D5310-2; | 4.90 | 18.92 | 68.17 | 4.98 |
| D5311, pSZ6317, | pH5; S8588, D5311-3; | 3.50 | 11.90 | 76.95 | 4.98 |
| BnOTE (D209A) | pH5; S8588, D5311-4; | 3.63 | 11.35 | 77.44 | 4.94 |
| | pH5; S8588, D5311-14; | 3.47 | 11.23 | 77.68 | 4.98 |
| | pH5; S8588, D5311-10; | 3.60 | 11.20 | 77.53 | 5.00 |
| | pH5; S8588, D5311-12; | 3.53 | 11.12 | 77.59 | 5.09 |
| D5312, pSZ6318, | pH5; S8588, D5312-20; | 4.79 | 37.97 | 47.74 | 6.01 |
| BnOTE (D124A, | pH5; S8588, D5312-40; | 5.97 | 22.94 | 62.20 | 5.11 |
| D209A) | pH5; S8588, D5312-39; | 6.07 | 22.75 | 62.24 | 5.17 |
| | pH5; S8588, D5312-16; | 5.25 | 18.81 | 67.36 | 5.09 |
| | pH5; S8588, D5312-26; | 4.93 | 18.70 | 68.37 | 4.96 |

### Example 9 Variant Garcinia mangostana Thioeserase

In this example, we demonstrate the ability to modify the activity and specificity of a FATA thioesterase originally isolated from Garcinia mangostana (GmFATA, accession 004792), using site directed mutagenesis targeting six amino acid positions within the enzyme and various combinations thereof. Facciotti et al (NatBiotech 1999) had previously altered three of the amino acids (G108, S111, V193). The remaining three amino acids targeted are L91, G96, and T156.

To test the impact of each mutation on the activity of the GmFATA, the wild-type and mutant genes were cloned into a vector enabling expression within the P. moriformis strain S3150. Table 27 summarizes the results from a three day lipid profile screen comparing the wild-type GmFATA with the 14 mutants. Three GmFATA mutants (DNA lot numbers D3998, D4000, D4003) increased the amount of C18:0 by at least 1.5 fold compared to the wild-type protein (DNA lot number D3997). D3998 and D4003 were mutations that had been described by Facciotti et al (NatBiotech 1999) as substitutions that increased the activity of the GmFATA. Strain S3150 expressing the mutations contained in DNA lot number D4000 was based on research at Solazyme which demonstrated this position influenced the activity of the FATB thioesterases. All of the constructs were codon optimized to reflect UTEX 1435 codon usage. Non-mutated GmFATA increases the fatty acid content of C18:0 and decreases the fatty acid content of C18:1 and C18:2. As can be seen in Table 27 the G90A mutant GmFATA increases the fatty acid content of C18:0 and decreases the fatty acid content of C18:1 and C18:2 when compared to the wild-type GmFATA.

TABLE 27

| Algal | | | | | | | |
|---|---|---|---|---|---|---|---|
| Strain | DNA # | GmFATA | C14:0 | C16:0 | C18:0 | C18:1 | C18:2 |
| P. | S3150 | | 1.63 | 29.82 | 3.08 | 55.95 | 7.22 |
| moriformis | D3997 | Wild-Type | 1.79 | 29.28 | 7.32 | 52.88 | 6.21 |
| S3150 | pSZ5083 | GmFATA | | | | | |
| | D3998 | S111A, | 1.84 | 28.88 | 11.19 | 49.08 | 6.21 |
| | pSZ5084 | V193A | | | | | |
| | D3999 | S111V, | 1.73 | 29.92 | 3.23 | 56.48 | 6.46 |
| | pSZ5085 | V193A | | | | | |
| | D4000 | G96A | 1.76 | 30.19 | 12.66 | 45.99 | 6.01 |
| | pSZ5086 | | | | | | |
| | D4001 | G96T | 1.82 | 30.60 | 3.58 | 55.50 | 6.28 |
| | pSZ5087 | | | | | | |
| | D4002 | G96V | 1.78 | 29.35 | 3.45 | 56.77 | 6.43 |
| | pSZ5088 | | | | | | |
| | D4003 | G108A | 1.77 | 29.06 | 12.31 | 47.86 | 6.08 |
| | pSZ5089 | | | | | | |
| | D4007 | G108V | 1.81 | 28.78 | 5.71 | 55.05 | 6.26 |
| | pSZ5093 | | | | | | |
| | D4004 | L91F | 1.76 | 29.60 | 6.97 | 53.04 | 6.13 |
| | pSZ5090 | | | | | | |
| | D4005 | L91K | 1.87 | 28.89 | 4.38 | 56.24 | 6.35 |
| | pSZ5091 | | | | | | |
| | D4006 | L91S | 1.85 | 28.06 | 4.81 | 56.45 | 6.47 |
| | pSZ5092 | | | | | | |
| | D4008 | T156F | 1.81 | 28.71 | 3.65 | 57.35 | 6.31 |
| | pSZ5094 | | | | | | |
| | D4009 | T156A | 1.72 | 29.66 | 5.44 | 54.54 | 6.26 |
| | pSZ5095 | | | | | | |
| | D4010 | T156K | 1.73 | 29.95 | 3.17 | 56.86 | 6.21 |
| | pSZ5096 | | | | | | |
| | D4011 | T156V | 1.80 | 29.17 | 4.97 | 55.44 | 6.27 |
| | pSZ5097 | | | | | | |

Nucleotide sequence of the GmFATA wild-type parental gene expression vector is shown below (D3997, pSZ5083). The plasmid pSZ5083 can be written as THI4a::CrTUB2-NeoR-PmPGH:PmSAD2-2Ver3-CpSAD1tp_GarmFATA1 FLAG-CvNR::THI4a. The 5′ and 3′ homology arms enabling targeted integration into the Thi4 locus are noted with lowercase; the CrTUB2 promoter is noted in uppercase italic which drives expression of the neomycin selection marker noted with lowercase italic followed by the PmPGH 3′UTR terminator highlighted in uppercase. The PmSAD2-1 promoter (noted in bold text) drives the expression of the GmFATA gene (noted with lowercase bold text) and is terminated with the CvNR 3′UTR noted in underlined, lower case bold. Restriction cloning sites and spacer DNA fragments are noted as underlined, uppercase plain lettering. The nucleotide sequence for all of the GmFATA constructs disclosed in this example is identical to that of pSZ5083 with the exception of the encoded GmFATA. The promoter, 3′UTR, selection marker and targeting arms are the same as described for pSZ5083. The individual GmFATA mutant sequences are shown below. The amino acid sequence of the unmutagenized GmFATA is showing in FIG. 1. The amino acid sequences of the altered GmFATA proteins are shown below.

SEQ ID NO: 135 pSZ5083
ccctcaactgcgacgctgggaaccttctccgggcaggcgatgtgcgtgggtttgcctccttg
gcacggctctacaccgtcgagtacgccatgaggcggtgatggctgtgtcggttgccacttcg
tccagagacggcaagtcgtccatcctctgcgtgtgtggcgcgacgctgcagcagtccctctg
cagcagatgagcgtgactttggccatttcacgcactcgagtgtacacaatccatttttctta
aagcaaatgactgctgattgaccagatactgtaacgctgatttcgctccagatcgcacagat
agcgaccatgttgctgcgtctgaaaatctggattccgaattcgaccctggcgctccatccat
gcaacagatggcgacacttgttacaattcctgtcacccatcggcatggagcaggtccactta
gattcccgatcacccacgcacatctcgctaatagtcattcgttcgtgtcttcgatcaatctc
aagtgagtgtgcatggatcttggttgacgatgcggtatgggtttgcgccgctggctgcaggg
tctgcccaaggcaagctaacccagctcctctccccgacaatactctcgcaggcaaagccggt
cacttgccttccagattgccaataaactcaattatggcctctgtcatgccatccatgggtct
gatgaatggtcacgctcgtgtcctgaccgttccccagcctctggcgtcccctgccccgccca
ccagcccacgccgcgcggcagtcgctgccaaggctgtctcggaGGTACCCTTTCTTGCGCTA
TGACACTTCCAGCAAAAGGTAGGGCGGGCTGCGAGACGGCTTCCCGGCGCTGCATGCAACAC
CGATGATGCTTCGACCCCCCGAAGCTCCTTCGGGGCTGCATGGGCGCTCCGATGCCGCTCCA
GGGCGAGCGCTGTTTAAATAGCCAGGCCCCCGATTGCAAAGACATTATAGCGAGCTACCAAA
GCCATATTCAAACACCTAGATCACTACCACTTCTACACAGGCCACTCGAGCTTGTGATCGCA
CTCCGCTAAGGGGGCGCCTCTTCCTCTTCGTTTCAGTCACAACCCGCAAACTCTAGAATATC
Aatgatcgagcaggacggcctccacgccggctcccccgccgcctgggtggagcgcctgttcg
gctacgactgggcccagcagaccatcggctgctccgacgccgccgtgttccgcctgtccgcc
cagggccgccccgtgctgttcgtgaagaccgacctgtccggcgccctgaacgagctgcagga
cgaggccgcccgcctgtcctggctggccaccaccggcgtgccctgcgccgccgtgctggacg
tggtgaccgaggccggccgcgactggctgctgctgggcgaggtgcccggccaggacctgctg
tcctcccacctggcccccgccgagaaggtgtccatcatggccgacgccatgcgccgcctgca
caccctggaccccgccacctgccccttcgaccaccaggccaagcaccgcatcgagcgcgccc
gcacccgcatggaggccggcctggtggaccaggacgacctggacgaggagcaccagggcctg
gcccccgccgagctgttcgcccgcctgaaggcccgcatgcccgacggcgaggacctggtggt
gacccacggcgacgcctgcctgcccaacatcatggtggagaacggccgcttctccggcttca
tcgactgcggccgcctgggcgtggccgaccgctaccaggacatcgccctggccacccgcgac
atcgccgaggagctgggcggcgagtgggccgaccgcttcctggtgctgtacggcatcgccgc
ccccgactcccagcgcatcgccttctaccgcctgctggacgagttcttctgaCAATTGACGC
CCGCGCGGCGCACCTGACCTGTTCTCTCGAGGGCGCCTGTTCTGCCTTGCGAAACAAGCCCC
TGGAGCATGCGTGCATGATCGTCTCTGGCGCCCCGCCGCGCGGTTTGTCGCCCTCGCGGGCG
CCGCGGCCGCGGGGGCGCATTGAAATTGTTGCAAACCCCACCTGACAGATTGAGGGCCCAGG
CAGGAAGGCGTTGAGATGGAGGTACAGGAGTCAAGTAACTGAAAGTTTTTATGATAACTAAC
AACAAAGGGTCGTTTCTGGCCAGCGAATGACAAGAACAAGATTCCACATTTCCGTGTAGAGG
CTTGCCATCGAATGTGAGCGGGCGGGCCGCGGACCCGACAAAACCCTTACGACGTGGTAAGA
AAAACGTGGCGGGCACTGTCCCTGTAGCCTGAAGACCAGCAGGAGACGATCGGAAGCATCAC
AGCACAGGATCCCGCGTCTCGAACAGAGCGCGCAGAGGAACGCTGAAGGTCTCGCCTCTGTC
GCACCTCAGCGCGGCATACACCACAATAACCACCTGACGAATGCGCTTGGTTCTTCGTCCAT
TAGCGAAGCGTCCGGTTCACACACGTGCCACGTTGGCGAGGTGGCAGGTGACAATGATCGGT
GGAGCTGATGGTCGAAACGTTCACAGCCTAGGGATATCGTGAAAACTCGCTCGACCGCCCGC
GTCCCGCAGGCAGCGATGACGTGTGCGTGACCTGGGTGTTTCGTCGAAAGGCCAGCAACCCC
AAATCGCAGGCGATCCGGAGATTGGGATCTGATCCGAGCTTGGACCAGATCCCCCACGATGC
GGCACGGGAACTGCATCGACTCGGCGCGGAACCCAGCTTTCGTAAATGCCAGATTGGTGTCC
GATACCTTGATTTGCCATCAGCGAAACAAGACTTCAGCAGCGAGCGTATTTGGCGGGCGTGC
TACCAGGGTTGCATACATTGCCCATTTCTGTCTGGACCGCTTTACCGGCGCAGAGGGTGAGT
TGATGGGGTTGGCAGGCATCGAAACGCGCGTGCATGGTGTGTGTGTCTGTTTTCGGCTGCAC
AATTTCAATAGTCGGATGGGCGACGGTAGAATTGGGTGTTGCGCTCGCGTGCATGCCTCGCC
CCGTCGGGTGTCATGACCGGGACTGGAATCCCCCCTCGCGACCCTCCTGCTAACGCTCCCGA
CTCTCCCGCCCGCGCGCAGGATAGACTCTAGTTCAACCAATCGACAACTAGTatggccaccg
catccactttctcggcgttcaatgcccgctgcggcgacctgcgtcgctcggcgggctccggg
ccccggcgcccagcgaggcccctccccgtgcgcgggcgcgccatccccccccgcatcatcgt
ggtgtcctcctcctcctccaaggtgaaccccctgaagaccgaggccgtggtgtcctccggcc
tggccgaccgcctgcgcctgggctccctgaccgaggacggcctgtcctacaaggagaagttc
atcgtgcgctgctacgaggtgggcatcaacaagaccgccaccgtggagaccatcgccaacct
gctgcaggaggtgggctgcaaccacgcccagtccgtgggctactccaccggcggcttctcca
ccacccccaccatgcgcaagctgcgcctgatctgggtgaccgcccgcatgcacatcgagatc
tacaagtaccccgcctggtccgacgtggtggagatcgagtcctggggccagggcgagggcaa
gatcggcacccgccgcgactggatcctgcgcgactacgccaccggccaggtgatcggccgcg
ccacctccaagtgggtgatgatgaaccaggacacccgccgcctgcagaaggtggacgtggac
gtgcgcgacgagtacctggtgcactgcccccgcgagctgcgcctggccttccccgaggagaa
caactcctccctgaagaagatctccaagctggaggacccctcccagtactccaagctgggcc
tggtgccccgccgcgccgacctggacatgaaccagcacgtgaacaacgtgacctacatcggc
tgggtgctggagtccatgccccaggagatcatcgacacccacgagctgcagaccatcaccct
ggactaccgccgcgagtgccagcacgacgacgtggtggactccctgacctcccccgagccct
ccgaggacgccgaggccgtgttcaaccacaacggcaccaacggctccgccaacgtgtccgcc
aacgaccacggctgccgcaacttcctgcacctgctgcgcctgtccggcaacggcctggagat
caaccgcggccgcaccgagtggcgcaagaagcccacccgcatggactacaaggaccacgacg
gcgactacaaggaccacgacatcgactacaaggacgacgacgacaagtgaATCGATgcagca
gcagctcggatagtatcgacacactctggacgctggtcgtgtgatggactgttgccgccaca
cttgctgccttgacctgtgaatatccctgccgcttttatcaaacagcctcagtgtgtttgat
cttgtgtgtacgcgcttttgcgagttgctagctgcttgtgctatttgcgaataccaccccca
gcatccccttccctcgtttcatatcgcttgcatcccaaccgcaacttatctacgctgtcctg
ctatccctcagcgctgctcctgctcctgctcactgcccctcgcacagccttggtttgggctc
cgcctgtattctcctggtactgcaacctgtaaaccagcactgcaatgctgatgcacgggaag
tagtgggatgggaacacaaatggaAAGCTTGAGCTCcagcgccatgccacgccctttgatgg
cttcaagtacgattacggtgttggattgtgtgtttgttgcgtagtgtgcatggtttagaata
atacacttgatttcttgctcacggcaatctcggcttgtccgcaggttcaaccccatttcgga
gtctcaggtcagccgcgcaatgaccagccgctacttcaaggacttgcacgacaacgccgagg
tgagctatgtttaggacttgattggaaattgtcgtcgacgcatattcgcgctccgcgacagc
acccaagcaaaatgtcaagtgcgttccgatttgcgtccgcaggtcgatgttgtgatcgtcgg
cgccggatccgccggtctgtcctgcgcttacgagctgaccaagcaccctgacgtccgggtac
gcgagctgagattcgattagacataaattgaagattaaacccgtagaaaaatttgatggtcg
cgaaactgtgctcgattgcaagaaattgatcgtcctccactccgcaggtcgccatcatcgag
cagggcgttgctcccggcggcggcgcctggctggggggacagctgttctcggccatgtgtgt
acgtagaaggatgaatttcagctggttttcgttgcacagctgtttgtgcatgatttgtttca
gactattgttgaatgtttttagatttcttaggatgcatgatttgtctgcatgcgact
SEQ ID NO: 136 Amino acid sequence of Gm FATA wild-type parental gene;
D3997, pSZ5083. The algal transit peptide is underlined and the FLAG epitope tag is
uppercase bold
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
SEQ ID NO:137 Amino acid sequence of Gm FATA S111A, V193A mutant gene;
D3998, pSZ5084. The algal transit peptide is underlined, the FLAG epitope tag is
uppercase bold and the S111A, V193A residues are lower-case bold.
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFaTTPTMRKLRLIWVTARMHIEIYK
YPAWSDVVEIESWGQGEGKIGTRRDWILRDYATGQVIGRATSKWVMMNQDTRRLQKVDaDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
SEQ ID NO:138 Amino acid sequence of Gm FATA S111V, V193A mutant gene;
D3999, pSZ5085. The algal transit peptide is underlined, the FLAG epitope tag is
uppercase bold and the S111V, V193A residues are lower-case bold.
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFvTTPTMRKLRLIWVTARMHIEIYK
YPAWSDVVEIESWGQGEGKIGTRRDWILRDYATGQVIGRATSKWVMMNQDTRRLQKVDaDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
SEQ ID NO: 139 Amino acid sequence of Gm FATA G96A mutant gene; D4000,
pSZ5086. The algal transit peptide is underlined, the FLAG epitope tag is uppercase
bold and the G96A residue is lower-case bold.
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVaCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
SEQ ID NO: 140 Amino acid sequence of Gm FATA G96T mutant gene; D4001,
pSZ5087. The algal transit peptide is underlined, the FLAG epitope tag is uppercase
bold and the G96T residue is lower-case bold.
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVtCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
SEQ ID NO: 141 Amino acid sequence of Gm FATA G96V mutant gene; D4002,
pSZ5088. The algal transit peptide is underlined, the FLAG epitope tag is uppercase
bold and the G96V residue is lower-case bold.
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVvCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
SEQ ID NO: 142 Amino acid sequence of Gm FATA G108A mutant gene;
D4003, pSZ5089. The algal transit peptide is underlined, the FLAG epitope tag is
uppercase bold and the G108A residue is lower-case bold.
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTaGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
SEQ ID NO: 143 Amino acid sequence of Gm FATA L91F mutant gene; D4004,
pSZ5090. The algal transit peptide is underlined, the FLAG epitope tag is uppercase
bold and the L91F residue is lower-case bold.
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANfLQEVGCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
SEQ ID NO: 144 Amino acid sequence of Gm FATA L91K mutant gene; D4005,
pSZ5091. The algal transit peptide is underlined, the FLAG epitope tag is uppercase
bold and the L91K residue is lower-case bold
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANkLQEVGCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
SEQ ID NO:145 FIG. 10. Amino acid sequence of Gm FATA L915 mutant
gene; D4006, pSZ5092. The algal transit peptide is underlined, the FLAG epitope tag is
uppercase bold and the L91S residue is lower-case bold
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANsLQEVGCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
SEQ ID NO: 146 Amino acid sequence of Gm FATA G108V mutant gene;
D4007, pSZ5093. The algal transit peptide is underlined, the FLAG epitope tag is
uppercase bold and the G108V residue is lower-case bold.
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTvGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
SEQ ID NO: 147 Amino acid sequence of Gm FATA T156F mutant gene;
D4008, pSZ5094. The algal transit peptide is underlined, the FLAG epitope tag is
uppercase bold and the T156F residue is lower-case bold.
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFSTTPTMRKLRLIWVTARMHIEIYK
YPAWSDVVEIESWGQGEGKIGfRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
SEQ ID NO: 148 Amino acid sequence of Gm FATA T156A mutant gene;
D4009, pSZ5095. The algal transit peptide is underlined, the FLAG epitope tag is
uppercase bold and the T156A residue is lower-case bold.
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFSTTPTMRKLRLIWVTARMHIEIYK
YPAWSDVVEIESWGQGEGKIGaRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
SEQ ID NO: 149 Amino acid sequence of Gm FATA T156K mutant gene; D4010,
pSZ5096. The algal transit peptide is underlined, the FLAG epitope tag is uppercase
bold and the T156K residue is lower-case bold.
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFSTTPTMRKLRLIWVTARMHIEIYK
YPAWSDVVEIESWGQGEGKIGkRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
SEQ ID NO: 150 Amino acid sequence of Gm FATA T156V mutant gene;
D4011, pSZ5097. The algal transit peptide is underlined, the FLAG epitope tag is
uppercase bold and the T156V residue is lower-case bold.
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFSTTPTMRKLRLIWVTARMHIEIYK
YPAWSDVVEIESWGQGEGKIGvRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
SEQ ID NO: 151 Nucleotide sequence of the GmFATA S111A, V193A mutant gene
(D3998, pSZ5084). The promoter, 3′UTR, selection marker and targeting arms are the same
as pSZ5083.
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttcgccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgcggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
SEQ ID NO: 152 Nucleotide sequence of the GmFATA S111V, V193A mutant gene
(D3999, pSZ5085). The promoter, 3′UTR, selection marker and targeting arms are the
same as pSZ5083.
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttcgtcaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgcggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
SEQ ID NO: 153 Nucleotide sequence of the GmFATA G96A mutant gene (D4000,
pSZ5086). The promoter, 3′UTR, selection marker and targeting arms are the same as
pSZ5083
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtggcgtgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
SEQ ID NO: 154 Nucleotide sequence of the GmFATA G96T mutant gene (D4001,
pSZ5087). The promoter, 3′UTR, selection marker and targeting arms are the same as
pSZ5083
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgacgtgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
SEQ ID NO: 155 Nucleotide sequence of the GmFATA G96V mutant gene (D4002,
pSZ5088). The promoter, 3′UTR, selection marker and targeting arms are the same as
pSZ5083.
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtggtgtgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
SEQ ID NO: 156 Nucleotide sequence of the GmFATA G108A mutant gene
(D4003, pSZ5089). The promoter, 3′UTR, selection marker and targeting arms are the
same as pSZ50836.
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccgcc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
SEQ ID NO: 157 Nucleotide sequence of the GmFATA L91F mutant gene (D4004,
pSZ5090). The promoter, 3′UTR, selection marker and targeting arms are the same as
pSZ5083
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacttcctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
SEQ ID NO: 158 Nucleotide sequence of the GmFATA L91K mutant gene (D4005,
pSZ5091). The promoter, 3′UTR, selection marker and targeting arms are the same as
pSZ5083.
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacaagctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
SEQ ID NO: 159 Nucleotide sequence of the GmFATA L91S mutant gene (D4006,
pSZ5092). The promoter, 3′UTR, selection marker and targeting arms are the same as
pSZ5083.
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaactcgctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
SEQ ID NO: 160 Nucleotide sequence of the GmFATA G108V mutant gene
(D4007, pSZ5093). The promoter, 3′UTR, selection marker and targeting arms are the
same as pSZ5083.
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccgtc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
SEQ ID NO: 161 Nucleotide sequence of the GmFATA T156F mutant gene (D4008,
pSZ5094). The promoter, 3′UTR, selection marker and targeting arms are the same as
pSZ5083.
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcttccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
SEQ ID NO: 162 Nucleotide sequence of the GmFATA T156A mutant gene (D4009,
pSZ5095). The promoter, 3′UTR, selection marker and targeting arms are the same as
pSZ5083
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcgcgcgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
SEQ ID NO: 163 Nucleotide sequence of the GmFATA T156K mutant gene (D4010,
pSZ5096). The promoter, 3′UTR, selection marker and targeting arms are the same as
pSZ5083.
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcaagcgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
SEQ ID NO: 164 Nucleotide sequence of the GmFATA T156V mutant gene (D4011,
pSZ5097). The promoter, 3′UTR, selection marker and targeting arms are the same as
pSZ5083
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcgtgcgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga

SEQUENCES
SEQ ID NO: 1

| | | | | | | |
|---|---|---|---|---|---|---|
| gcgaggggtc | tgcctgggcc | agccgctccc | tctgaacacg | ggacgcgtgg | tccaattcgg | 60 |
| gcttcgggac | cctttggcgg | tttgaacgcc | tgggagaggg | cgcccgcgag | cctggggacc | 120 |
| ccggcaacgg | cttccccaga | gcctgccttg | caatctcgcg | cgtcctctcc | ctcagcacgt | 180 |
| ggcggttcca | cgtgtggtcg | ggcgtcccgg | actagctcac | gtcgtgacct | agcttaatga | 240 |
| acccagccgg | gcctgcagca | ccaccttaga | ggttttgatt | atttgattag | accaatctat | 300 |
| tcacc | | | | | | 305 |

SEQ ID NO: 2

| | | | | | | |
|---|---|---|---|---|---|---|
| ggcgaataga | ttggtataat | gaaataatca | aaacctctta | ggcggtgcta | caggcccggc | 60 |
| tgggttcatt | aagctaggtc | acgacgcgag | ctagtccggg | aagcccgacc | acacgtggaa | 120 |
| ccgccacgtg | ctgagggaga | ggacgcgcga | gattgcaagg | caggctctgg | ggaagccgtt | 180 |
| gccggggtcc | ccaggctcgc | gggcgcccca | tccctggcgt | tcaaaccgcc | aaagggtccc | 240 |
| gaagcccgaa | ttggaccacg | cgtcccgtgt | ttagagggag | cggctggccc | aggcagaccc | 300 |
| ctcgc | | | | | | 305 |

SEQ ID NO: 3

| | | | | | | |
|---|---|---|---|---|---|---|
| ggtgaataga | ttggtctaat | caaataatca | aaacctctaa | ggtggtgctg | caggcccggc | 60 |
| tgggttcatt | aagctaggtc | acgacgtgag | ctagtccggg | acgcccgacc | acacgtggaa | 120 |
| ccgccacgtg | ctgagggaga | ggacgcgcga | gattgcaagg | caggctctgg | ggaagccgtt | 180 |
| gccggggtcc | ccaggctcgc | gggcgccctc | tcccaggcg | tcaaaccgcc | aaagggtccc | 240 |
| gaagcccgaa | ttggaccacg | cgtcccgtgt | tcagagggag | cggctggccc | aggcagaccc | 300 |
| ctcgc | | | | | | 305 |

SEQ ID NO: 4

| | | | | | | |
|---|---|---|---|---|---|---|
| gtgatgggtt | ctttagacga | tccagcccag | gatcatgtgt | tgcccacatg | gagcctatcc | 60 |
| acgctggcct | agaaggcaag | cacatttcaa | ggtgaaccca | cgtccatgga | gcgatggcgc | 120 |
| caatatctcg | cctctagacc | aagcggttct | caccccaact | gcgtcatttg | tatgtatggc | 180 |
| tgcaaagttg | tcggtacgat | agaggccgcc | aacctggcgg | cgagggcgag | gagctggttg | 240 |
| ccgatctgtg | cccaagcatg | tgtcggagct | cggctgtctc | ggcagcgagc | tcctgtgcaa | 300 |
| ggggcttgca | tcgagaatgt | caggcgatag | acactgcacg | ttggggacac | ggaggtgccc | 360 |
| ctgtggcgtg | tcctggatgc | cctcgggtcc | gtcgcgagaa | gctctggcga | ccagcacccg | 420 |
| gccacaaccg | cagcaggcgt | tcacccacaa | gaatcttcca | gatcgtgatg | cgcatgtatc | 480 |
| gtgacacgat | tggcgaggtc | cgcaggacgc | acacggactc | gtccactcat | cagaactggt | 540 |
| cagggcaccc | atctgcgtcc | cttttcagga | accacccacc | gctgccaggc | accttcgcca | 600 |
| gcggcggact | ccacacagag | aatgccttgc | tgtgagagac | catggccggc | aagtgctgtc | 660 |
| ggatctgccc | gcatacggtc | agtccccagc | acaaggaagc | caagagtaca | ggctgttggt | 720 |
| gtcgatggag | gagtggccgt | tcccacaagt | agtgagcggc | agctgctcaa | cggcttcccc | 780 |
| ctgttcatct | tggcaaagcc | agtgacttcc | tacaagtatg | tgatgcagat | cggcactgca | 840 |
| atctgtcggc | atgcgtacag | aacatcggct | cgccagggca | gcgttgctcg | ctctggatga | 900 |
| gctgcttggg | aggaatcatc | ggcacacgcc | cgtgccgtgc | ccgcgccccg | cgcccgtcgg | 960 |
| gaaaggcccc | cggttaggac | actgccgcgt | cagccagtcg | tgggatcgat | cggacgtggc | 1020 |
| gaatcctcgc | ccggacaccc | tcatcacacc | ccacatttcc | ctgcaagcaa | tcttgccgac | 1080 |
| aaaatagtca | agatccattg | ggtttaggga | acacgtgcga | gactgggcag | ctgtatctgt | 1140 |
| ccttgccccg | cgtcaaattc | ctgggcgtga | cgcagtcaca | ggagaatcta | ttagaccctg | 1200 |
| gacttgcagc | tcagtcatgg | gcgtgagtgg | ctaaagcacc | taggtcaggc | gagtaccgcc | 1260 |
| ccttccccag | gattcactct | tctgcgattg | acgttgagcc | tgcatcgggc | tgcttcgtca | 1320 |
| cc | | | | | | 1322 |

SEQ ID NO: 5

| | | | | | | |
|---|---|---|---|---|---|---|
| tcggagctaa | agcagagact | ggacaagact | tgcgttcgca | tactggtgac | acagaatagc | 60 |
| tcccatctat | tcatacgcct | ttgggaaaag | gaacgagcct | tgtggcctct | gcattgctgc | 120 |
| ctgctttgag | gccgaggacg | gtgcgggacg | ctcagatcca | tcagcgatcg | ccccaccctc | 180 |
| agagcacctc | cgatccaagg | caatactatc | aggcaaagtt | tccaaattca | aacattccaa | 240 |
| aatcacgcca | gggactggat | cacacacgca | gatcagcgcc | gttttgctct | ttgcctacgg | 300 |
| gcgactgtgc | cacttgtcga | cccctggtga | cgggagggac | cacgcctgcg | gttggcatcc | 360 |
| acttcgacgg | acccagggac | ggtttctcat | gccaaacctg | agatttgagc | acccagatga | 420 |
| gcacattatg | cgttttagga | tgcctgagca | gcgggcgtgc | aggaatctgg | tctcgccaga | 480 |
| ttcaccgaag | atgcgcccat | cggagcgagg | cgagggcttt | gtgaccacgc | aaggcagtgt | 540 |
| gaggcaaaca | catagggaca | cctgcgtctt | tcaatgcaca | gacatctatg | gtgcccatgt | 600 |
| atataaaatg | ggctacttct | gagtcaaacc | aacgcaaact | gcgctatggc | aaggccggcc | 660 |
| aaggttggaa | tcccggtctg | tctggatttg | agtttgtggg | ggctatcacg | tgacaatccc | 720 |
| tgggattggg | cggcagcagc | gcacggcctg | ggtggcaatg | gcgcactaat | actgctgaaa | 780 |
| gcacggctct | gcatcccttt | ctcttgacct | gcgattggtc | cttttcgcaa | gcgtgatcat | 840 |
| c | | | | | | 841 |

SEQ ID NO: 6

| | | | | | | |
|---|---|---|---|---|---|---|
| tcggagctaa | agcagaaact | gaacaagact | tgcgttcgca | tacttgtgac | actgaatagg | 60 |
| ttcaatctat | tcatacgcct | ttgggaaact | gaacgagcct | tgtggcctct | gcattgctgc | 120 |
| ctgctttgag | gccgaggacg | gcgcggaacg | cacagatcca | tcagcgatcg | ccccaccctc | 180 |
| agagtacatc | cgatccaagg | caatactatc | aggcaaagtt | tccaaattca | aacattccaa | 240 |
| aattacgtca | gggactggat | cacacacgca | gatcagcgcc | gttttgctct | ttgcctacgg | 300 |
| gcgactgtgc | cacttgtcga | cgcctggtga | cgggagggac | cacgcctgcg | gttggcatcc | 360 |
| acttcgacgg | acccagggac | ggtctcacat | gccaaacctg | agatttgagc | accaagatga | 420 |
| gcacattatg | cgtttttgga | tgcctgagca | gcgggcgtgc | aggaatctgg | tctcgccaga | 480 |
| ttcaccgaag | atgcggccat | cggagcgagg | cgagggctgt | gtggccacgc | caggcagtgt | 540 |
| gaggcaaaca | cacagggaca | tctgcttctt | tcgatgcaca | gacatctatg | ttgcccgtgc | 600 |
| atataaaatg | ggctacttct | gaatcaaacc | aacgcaaact | tcgctatggc | aaggccggcc | 660 |
| aaggttggaa | tcccggtctg | tctggatttg | agtttgtggg | ggctatcacg | tgacaatccc | 720 |
| tgggattggg | cggcagcagc | gcacggcctg | gatggcaatg | gcgcactaat | actgctgaaa | 780 |
| gcacggctct | gcatcccttt | ctcttgacct | gcgattggtc | cttttcgcaa | gcgtgatcat | 840 |
| c | | | | | | 841 |

SEQ ID NO: 7

| | | | | | | |
|---|---|---|---|---|---|---|
| caccgatcac | tccgtcgccg | cccaagagaa | atcaacctcg | atggagggcg | aggtggatca | 60 |
| gaggtattgg | ttatcgttcg | ttcttagtct | caatcaatcg | tacaccttgc | agttgcccga | 120 |
| gtttctccac | acatacagca | cctcccgctc | ccagcccatt | cgagcgaccc | aatccgggcg | 180 |
| atcccagcga | tcgtcgtcgc | ttcagtgctg | accggtggaa | agcaggagat | ctcgggcgag | 240 |
| caggaccaca | tccagcccag | gatcttcgac | tggctcagag | ctgaccctca | cgcggcacag | 300 |
| caaaagtagc | acgcacgcgt | tatgcaaact | ggttacaacc | tgtccaacag | tgttgcgacg | 360 |
| ttgactggct | acattgtctg | tctgtcgcga | gtgcgcctgg | gcccttacgg | tgggacactg | 420 |
| gaactccgcc | ccgagtcgaa | cacctagggc | gacgcccgca | gcttggcatg | acagctctcc | 480 |
| ttgtgttcta | aataccttgc | gcgtgtggga | ga | | | 512 |

SEQ ID NO: 8

| | | | | | | |
|---|---|---|---|---|---|---|
| atccaccgat | cactccgtcg | ccgcccaaga | gaattcaacc | tcgatggagg | gcaaggtgga | 60 |
| tcagaggtat | tggttatcgt | tcgctattag | tctcaatcaa | tcgtgcacct | tgcagttgct | 120 |
| cgagtttctc | cacacataca | gcacctcccg | ctcccagccc | attcgagcga | cccaatccgg | 180 |
| gcgatcccag | cgatcgtcgt | cgcttcagtg | ctgaccggtg | gaaagcagga | gatctcgggc | 240 |
| gagcaggacc | acatccagca | caggatcttc | gactggctca | gagctgaccc | tcacgcggca | 300 |
| cagcaaaagt | agcccgcacg | cgttatgcaa | acaggttaca | acctgtccaa | cactgttgcg | 360 |
| acgttgactg | gctacattgt | ctgtctgtcg | cgagtacgcc | tggaccctta | cggtgggaca | 420 |
| ctggaactcc | gccccgagtc | gaacacctag | ggcgacgccc | gcagcttggc | atgacagctc | 480 |
| tccttgtatt | ctaaatacct | cgcgcgtgtg | ggagaa | | | 516 |

SEQ ID NO: 9

| | | | | | | |
|---|---|---|---|---|---|---|
| atgatgcgcg | tgtacgacta | tcaaggaaga | aagaggactt | aatttcttac | cttctaacca | 60 |
| ccatattctt | tttgctggat | gcttgctcgt | ctcgatgaca | attgtgaacc | tcttgtgtga | 120 |
| ccctgaccct | gctgcaaggc | tctccgaccg | cacgcaaggc | gcagccggcg | cgtccggagg | 180 |
| cgatcggatc | caatccagtc | gtcctcccgc | agcccgggca | cgtttgccca | tgcaggccct | 240 |
| tccacaccgc | tcaagagact | cccgaacacc | gcccactcgg | cactcgcttc | ggctgccgag | 300 |
| tgcgcgtttg | agtttgccct | gccacagaag | acacc | | | 335 |

SEQ ID NO: 10

| | | | | | | |
|---|---|---|---|---|---|---|
| atgatgcgcg | tgtacgacta | tcaaggaaga | aagaggactt | aatttcttac | cttctaacca | 60 |
| ccatattctt | tttgctggat | gcttgctcgt | ctcgatgaca | attgtgaacc | tcttgtgtga | 120 |
| ccctgaccct | gctgcaaggc | tctccgaccg | cacgcaaggc | gcagccggcg | cgtccggagg | 180 |
| cgatcggatc | caatccagtc | gtcctcccgc | agcccgggca | cgtttgccca | tgcaggccct | 240 |
| tccacaccgc | tcaagagact | cccgaacacc | gcccactcgg | cactcgcttc | ggctgccgag | 300 |
| tgcgcgtttg | agtttgccct | gccacaggag | acatc | | | 335 |

SEQ ID NO: 11

| | | | | | | |
|---|---|---|---|---|---|---|
| cccgggcgag | ctgtacgcct | acggagcgag | gcctggtgtg | accgttgcga | tctcgccagc | 60 |
| agacgtcgcg | gagcctcgtc | ccaaaggccc | tttctgatcg | agcttgtcgt | ccactggacg | 120 |
| ctttaagttg | cgcgcgcgat | gggataaccg | agctgatctg | cactcagatt | ttggtttgtt | 180 |
| ttcgcgcatg | gtgcagcgag | gggaggtact | acgctggggt | acgagatcct | ccggattccc | 240 |
| agaccgtgtt | gccggcattt | acccggtcat | cgccagcgat | tcgggacgac | aaggccttat | 300 |
| cctgtgctga | gacgctcgag | cacgtttata | aaattgtggg | taccgcggta | tgcacagcgt | 360 |
| tcaacacgcg | ccacgccgaa | attggttggt | gggggagcac | gtatgggact | gacgtatggc | 420 |
| cagcagcgaa | cactcaccga | acaagtgcca | atgtatacct | tgcatcaatg | atgctccggc | 480 |
| agcttcgatt | gactgtctcg | aaaaagtgtg | agcaagcaga | tcatgtggcc | gctctgtcgc | 540 |
| gcagcacctg | acgcattcga | cacccacggc | aatgcccagg | ccagggaata | gagagtaaga | 600 |
| caactcccat | tgttcagcaa | aacattgcac | tgcagtgcct | tcacaactat | acaatgaatg | 660 |
| ggagggaata | tgggctctgc | atgggacagc | ttagctggga | cattcggcta | ctgaacaaga | 720 |
| aaaccccacg | agaaccaatt | ggcgaaacct | gccgggagga | ggtgatcgtt | tctgtaaatg | 780 |
| gcttacgcat | tcccccccgg | cggctcacga | ggggtgtggt | gaaccctgcc | agctgatcaa | 840 |
| gtgcttgctg | acgtcggcca | gggaggtgta | tgtgattggg | ccgtggggcg | tgagttatcc | 900 |
| taccgccgga | cccgcgaagt | cacatgacga | atggccgtgc | gggatgacga | gagcacgact | 960 |
| cgctctttct | tcgccggccc | ggcttcatgg | aggacaataa | taaagggtgg | ccaccggcaa | 1020 |
| cagccctcca | tacctgaacc | gattccagac | ccaaacctct | tgaattttga | gggatccagt | 1080 |
| tcaccggtat | agtcacg | | | | | 1097 |

SEQ ID NO: 12

| | | | | | | |
|---|---|---|---|---|---|---|
| atccccgggc | gagctgtacg | cctacggagc | gaggcctggt | gtgaccgttg | cgatctcgcc | 60 |
| agcagacgtc | gcggagcctc | gtcccaaagg | ccctttctga | tcgagcttgt | cgtccactgg | 120 |
| acgctttaag | ttgcgcgcgc | gatgggataa | ccgagctgat | ctgcactcag | attttggttt | 180 |
| gttttcgcgc | atggtgcagc | gaggggaggt | actacgctgg | ggtacgagat | cctccggatt | 240 |
| cccagaccgt | gttgccggca | tttacccggt | catcgccagc | gattcgggac | gacaaggcct | 300 |
| tatcctgtgc | tgagacgctc | gagcacgttt | ataaaattgt | ggtcaccgtg | gtacgcacag | 360 |
| cgtccaacac | gcgccacgcc | gaaattcgtt | ggtgggggag | cacgtatcgg | actgacgtat | 420 |
| ggccagcagc | gaacactcac | caaacaggtg | ccaatgtata | gcttgcatca | atgatgctct | 480 |
| ggcagcttcg | attgactgtc | tcgaaaaagt | gtgtgcaaac | agattatgtg | gccgctctgt | 540 |
| ggccgcgcag | cacctgacgc | actcgacacc | cacggcaatg | cccaggccaa | ggaacagaga | 600 |
| gtaagacaac | tcccattgtt | cagtaaaaca | ttgcactgca | gtgccttcac | aaacatacaa | 660 |
| cgaatgggag | ggaatatggg | cttcgaatgg | gacagcttag | ctgggacatt | cggttactga | 720 |
| acaagaaaac | cccacgagaa | ccaactggcg | aaacctgccg | ggaggaggtg | atcgtttttg | 780 |
| taaatggctt | acgcattccc | cccccggcgg | ctcacggggg | gtgtggtgaa | ccctgccagc | 840 |
| tgatcaagtg | cttgctgacg | tcggccaggg | aggtgtatgt | gatttggccg | tggggcgtga | 900 |
| gttatcctac | cgccggaccc | gcgaagtcac | atgacgaatg | gccgtgcggg | atgacgagag | 960 |
| cagggctcgc | tctttcttcg | ccggcccggc | ttcatggagg | acaataataa | agggtggcca | 1020 |
| ccggcaacag | ccctccatac | ctgaaccga | ttccagaccca | aacctcttga | attttgaggg | 1080 |
| atccagttca | ccggtatagt | cacga | | | | 1105 |

SEQ ID NO: 13

| | | | | | | |
|---|---|---|---|---|---|---|
| gcgagtggtt | ttgctgccgg | gaagggagtg | gggagcgtcg | agcgagggac | gcggcgctcg | 60 |
| aggcgcacgt | cgtctgtcaa | cgcgcgcggc | cctcgcggcc | cgcggcccca | cccagctcta | 120 |
| atcatcgaaa | actaagaggc | tccacacgcc | tgtcgtagaa | tgcatgggat | tcgccagtag | 180 |
| accacgatct | gcgccgaaga | agctggtcta | cccgacgttt | tttgttgctc | ctttattctg | 240 |
| aatgatatga | agatagtgtg | cgcagtgcca | cgcataggca | tcaggagcaa | gggaggacgg | 300 |
| gtcaacttga | aagaaccaaa | ccatccatcc | gagaaatgcg | catcatcttt | gtagtaccat | 360 |
| caaacgcctt | ggccaatgtc | ttctgcatgg | acaacacaac | ctgctcctgg | ccacacggtc | 420 |
| gacttggagc | gccccatgcg | cccaggtcgc | cacgacccgc | ggcccagcgc | gcggcgattc | 480 |
| gcctcacgag | atcccggcgg | acccggcacg | cccgcgggcc | gacggtgcgc | ttggcgatgc | 540 |
| tgctcattaa | cccacggccg | tcacccgatc | cacatgctct | ttttcaacac | atccacattg | 600 |
| gaatagagct | ctaccagggt | gagtactgca | ttctttgggg | ctgggaggac | cccactcgac | 660 |
| acctggtcct | tcatcggccg | aaagcccgaa | cctgagcgct | tccccgcccc | gttcctcatc | 720 |
| cccgactttc | cgatggccca | ttgcagtttc | aaac | | | 754 |

SEQ ID NO: 14

| | | | | | | |
|---|---|---|---|---|---|---|
| atctgggtgg | aggactggga | gtaagatgta | aggatattaa | ttaaacattc | tagtttgttg | 60 |
| atggcacaac | agtcaatgca | tttcagtcgt | cttgctcctt | ataacctatg | cgtgtgccat | 120 |
| cgccggccat | gcacctgtgg | cgtggtaccg | accatcgggg | agaggcccga | gattcggagg | 180 |
| tacctcccgc | cctgggcgag | cccttcacgt | gacggcacaa | gtcccttgca | tcggcccgcg | 240 |
| agcacggaat | acagagcccc | gtgcccccca | cgggccctca | catcatccac | tccattgttc | 300 |
| ttgccacacc | gatcagca | | | | | 318 |

SEQ ID NO: 15

| | | | | | | |
|---|---|---|---|---|---|---|
| tgggtggagg | actgggaaga | agatgtaagg | atatcaattt | aacattctag | tttgttgatg | 60 |
| gcacaacagt | cactgaatac | cgggcgtctg | gctgctaaaa | tagccggagc | gtgtgccatc | 120 |
| gccggccatg | catctgtggc | gtggtaccga | ccatcaggga | gaggcccgag | attcggaggt | 180 |
| acctcccgcc | ctgggcgagc | ccttcacgtg | acggcacaag | tcccttgcat | cggcccgcga | 240 |
| gcacggaata | cagagccccg | tgctccccac | gggccctcac | atcatccact | ccattgttct | 300 |
| tgccacaccg | atcagc | | | | | 316 |

SEQ ID NO: 16

| | | | | | | |
|---|---|---|---|---|---|---|
| ataacgaggc | acaatgatcg | atatttctat | cgaacaactg | tatttagccc | tgtacgtacc | 60 |
| ccgctcttgg | gccagcccgt | ccgtgcttgc | cttcggaaaa | ttgcatggcg | cctcatgcaa | 120 |
| actcgcgctc | tcacagcaga | tctcgcccag | ctcccgggag | agcaatcgcg | ggtggggccc | 180 |
| ggggcgaatc | caggacgcgc | cccgcggggc | cgctccactc | gccagggcca | atgggcggct | 240 |
| tatagtcctg | gcatgggctc | tgcatgcaca | gtatcgcagt | ttgggcgagg | tgttgccccc | 300 |

| | | | | |
|---|---|---|---|---|
| gcgatttcga | atacgcgacg | cccggtactc | gtgcgagaac | agggttcttg |

Prototheca moriformis (UTEX 1435)Amt02 promoter
SEQ ID NO: 17
TCACCAGCGGACAAAGCACCGGTGTATCAGGTCCGTGTCATCCACTCTAAAGAGCTCGACTACGACCTACTGATG
GCCCTAGATTCTTCATCAAAAACGCCTGAGACACTTGCCCAGGATTGAAACTCCCTGAAGGGACCACCAGGGGCC
CTGAGTTGTTCCTTCCCCCCGTGGCGAGCTGCCAGCCAGGCTGTACCTGTGATCGGGGCTGGCGGGAAAACAGGC
TTCGTGTGCTCAGGTTATGGGAGGTGCAGGACAGCTCATTAAACGCCAACAATCGCACAATTCATGGCAAGCTAA
TCAGTTATTTCCCATTAACGAGCTATAATTGTCCCAAAATTCTGGTCTACCGGGGGTGATCCTTCGTGTACGGGC
CCTTCCCTCAACCCTAGGTATGCGCACATGCGGTCGCCGCGCAACGCGCGCGAGGGCCGAGGGTTTGGGACGGGC
CGTCCCGAAATGCAGTTGCACCCGGATGCGTGGCACCTTTTTTGCGATAATTTATGCAATGGACTGCTCTGCAAA
ATTCTGGCTCTGTCGCCAACCCTAGGATCAGCGGTGTAGGATTTCGTAATCATTCGTCCTGATGGGGAGCTACCG
ACTGCCCTAGTATCAGCCCGACTGCCTGACGCCAGCGTCCACTTTTGTGCACACATTCCATTCGTGCCCAAGACA
TTTCATTGTGGTGCGAAGCGTCCCCAGTTACGCTCACCTGATCCCCAACCTCCTTATTGTTCTGTCGACAGAGTG
GGCCCAGAGGCCGGTCGCAGCC
Prototheca moriformis (UTEX 1435) Amt03 promoter
SEQ ID NO: 18
Ggccgacaggacgcgcgtcaaaggtgctggtcgtgtatgccctggccggcaggtcgttgctgctgctggttagtg
attccgcaaccctgattttggcgtcttattttggcgtggcaaacgctggcgcccgcgagccgggccggcggcgat
gcggtgccccacggctgccggaatccaagggaggcaagagcgcccgggtcagttgaagggctttacgcgcaaggt
acagccgctcctgcaaggctgcgtggtggaattggacgtgcaggtcctgctgaagttcctccaccgcctcaccag
cggacaaagcaccggtgtatcaggtccgtgtcatccactctaaagagctcgactacgacctactgatggccctag
attcttcatcaaaaacgcctgagacacttgcccaggattgaaactccctgaagggaccaccaggggccctgagtt
gttccttccccccgtggcgagctgccagccaggctgtacctgtgatcgaggctggcgggaaaataggcttcgtgt
gctcaggtcatgggaggtgcaggacagctcatgaaacgccaacaatcgcacaattcatgtcaagctaatcagcta
tttcctcttcacgagctgtaattgtcccaaaattctggtctaccgggggtgatccttcgtgtacgggcccttccc
tcaaccctaggtatgcgcgcatgcggtcgccgcgcaactcgcgcgagggccgagggtttgggacgggccgtcccg
aaatgcagttgcacccggatgcgtggcaccttttttgcgataatttatgcaatggactgctctgcaaaattctgg
ctctgtcgccaaccctaggatcagcggcgtaggatttcgtaatcattcgtcctgatggggagctaccgactaccc
taatatcagcccgactgcctgacgccagcgtccacttttgtgcacacattccattcgtgcccaagacatttcatt
gtggtgcgaagcgtccccagttacgctcacctgtttcccgacctccttactgttctgtcgacagagcgggcccac
aggccggtcgcagcc
pSZ3840/D2554 transforming construct (CpauLPAAT1)
SEQ ID NO: 19
gctcttccgctaacggaggtctgtcaccaaatggaccccgtctattgcgggaaaccacggcgatggcacgtttcaaaacttgatga
aatacaatattcagtatgtcgcgggcggcgacggcggggagctgatgtcgcgctgggtattgcttaatcgccagcttcgcccccgt
cttggcgcgaggcgtgaacaagccgaccgatgtgcacgagcaaatcctgacactagaagggctgactcgcccggcacggctgaa
ttacacaggcttgcaaaaataccagaatttgcacgcaccgtattcgcggtattttgttggacagtgaatagcgatgcggcaatggc
ttgtggcgttagaaggtgcgacgaaggtggtgccaccactgtgccagccagtcctggcggctcccagggccccgatcaagagcca
ggacatccaaactacccacagcatcaacgccccggcctatactcgaaccccacttgcactctgcaatggtatgggaaccacgggg

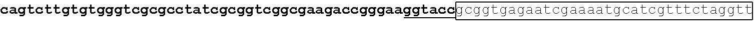

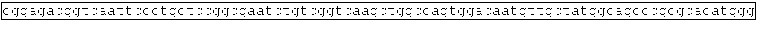

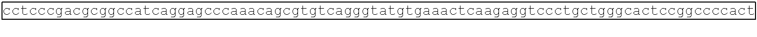

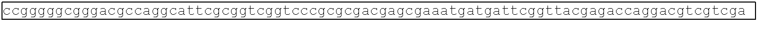

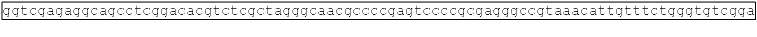

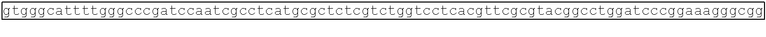

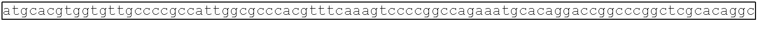

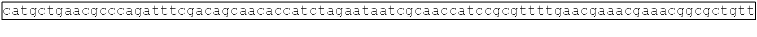

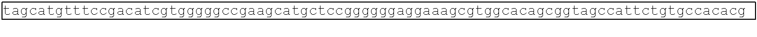

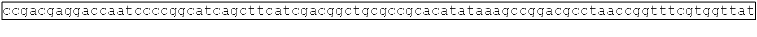

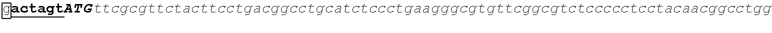

gcctgacgccccagatgggctgggacaactggaacacgttcgcctgcgacgtctccgagcagctgctgctggacaggccga
ccgcatctccgacctgggcctgaaggacatgggctacaagtacatcatcctggacgactgctggtcctccggccgcgactccga
cggcttcctggtcgccgacgagcagaagttccccaacggcatgggccacgtcgccgaccacctgcacaacaactccttcctgtt
cggcatgtactcctccgcgggcgagtacacgtgcgccggctaccccggctccctgggccgcgaggaggaggacgcccagact
tcgcgaacaaccgcgtggactacctgaagtacgacaactgctacaacaagggccagttcggcacgcccgagatctcctacca
ccgctacaaggccatgtccgacgccctgaacaagacgggccgccccatcttctactccctgtgcaactggggccaggacctga
ccactactggggctccggcatcgcgaactcctggcgcatgtccggcgacgtcacggcggagttcacgcgccccgactcccgct
gcccctgcgacggcgacgagtacgactgcaagtacgccggcttccactgctccatcatgaacatcctgaacaaggccgccccc
atgggccagaacgcgggcgtcggcggctggaacgacctggacaacctggaggtcggcgtcggcaacctgacggacgacga
ggagaaggcgcacttctccatgtgggccatggtgaagtcccccctgatcatcggcgcgaacgtgaacaacctgaaggcctcct
cctactccatctactcccaggcgtccgtcatcgccatcaaccaggactccaacggcatccccgccacgcgcgtctggcgctacta
cgtgtccgacacggacgagtacggccagggcgagatccagatgtggtccggccccctggacaacggcgaccaggtcgtggc
gctgctgaacggcggctccgtgtcccgccccatgaacacgaccctggaggagatcttcttcgactccaacctgggctccaagaa
gctgacctccacctgggacatctacgacctgtgggcgaaccgcgtcgacaactccacggcgtccgccatcctgggccgcaaca
agaccgccaccggcatcctgtacaacgccaccgagcagtcctacaaggacggcctgtccaagaacgacacccgcctgttcgg
ccagaagatcggctccctgtcccccaacgcgatcctgaacacgaccgtccccgcccacggcatcgcgttctaccgcctgcgcccc

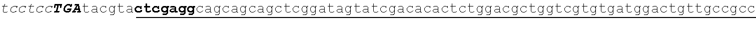

acacttgctgccttgacctgtgaatatccctgccgcttttatcaaacagcctcagtgtgtttgatcttgtgtgtacgcgcttttgcgagtt
gctagctgcttgtgctatttgcgaataccacccccagcatccccttccctcgtttcatatcgcttgcatcccaaccgcaacttatctacg
ctgtcctgctatccctcagcgctgctcctgctcctgctcactgcccctcgcacagccttggtttgggctccgcctgtattctcctggtact

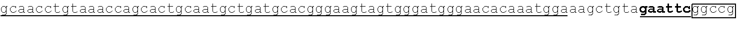

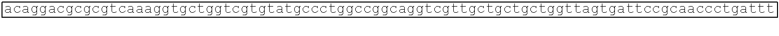

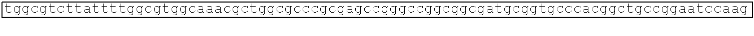

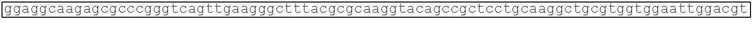

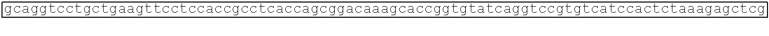

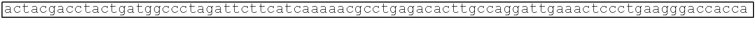

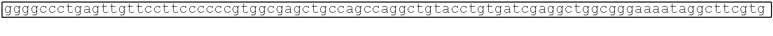

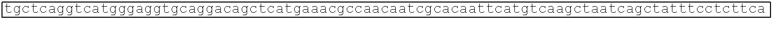

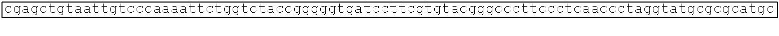

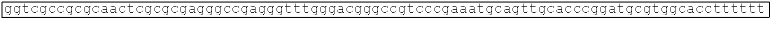

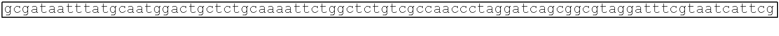

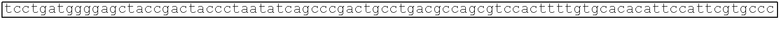

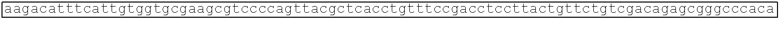

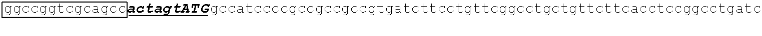

atcaacctgttccaggccctgtgcttcgtgctggtgtggcccctgtccaagaacgcctaccgccgcatcaaccgcgtgttcgccg
agctgctgctgtccgagctgctgtgcctgttcgactggtgggccggcgccaagctgaagctgttcaccgaccccgagaccttcc
gcctgatgggcaaggagcacgccctggtgatcatcaaccacatgaccgagctggactggatgctgggctgggtgatgggcca
gcacctgggctgcctgggctccatcctgtccgtggccaagaagtccaccaagttcctgcccgtgctgggctggtccatgtggttct
ccgagtacctgtacatcgagcgctcctgggccaaggaccgcaccaccctgaagtcccacatcgagcgcctgaccgactacccc
ctgcccactggatggtgatcttcgtggagggcacccgcttcacccgcaccaagctgctggccgcccagcagtacgccgcctcct
ccggcctgcccgtgccccgcaacgtgctgatcccccgcaccaagggcttcgtgtcctgcgtgtcccacatgcgctccttcgtgccc
gccgtgtacgacgtgaccgtggccttccccaagacctcccccccccccaccctgctgaacctgttcgagggccagtccatcgtgc
tgcacgtgcacatcaagcgccacgccatgaaggacctgcccgagtccgacgacgccgtggcccagtggtgccgcgacaagtt
cgtggagaaggacgccctgctggacaagcacaacgccgaggacaccttctccggccaggaggtgcaccgcaccggctcccg
ccccatcaagtccctgctggtggtgatctcctgggtggtggtgatcaccttcggcgccctgaagttcctgcagtggtcctcctgga
agggcaaggccttctccgtgatcggcctgggcatcgtgaccctgctgatgcacatgctgatcctgtcctcccaggccgagcgctc

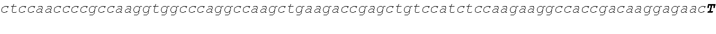

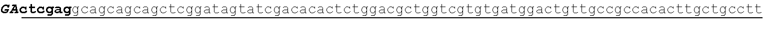

gacctgtgaatatccctgccgcttttatcaaacagcctcagtgtgtttgatcttgtgtgtacgcgcttttgcgagttgctagctgcttgtg
ctatttgcgaataccacccccagcatccccttccctcgtttcatatcgcttgcatcccaaccgcaacttatctacgctgtcctgctatcc
ctcagcgctgctcctgctcctgctcactgcccctcgcacagccttggtttgggctccgcctgtattctcctggtactgcaacctgtaaac
cagcactgcaatgctgatgcacgggaagtagtgggatgggaacacaaatggaaagcttgagctcagcggcgacggtcctgctacc
gtacgacgttgggcacgcccatgaaagtttgtataccgagcttgttgagcgaactgcaagcgcggctcaaggatacttgaactcct
ggattgatatcggtccaataatggatggaaaatccgaacctcgtgcaagaactgagcaaacctcgttacatggatgcacagtcgc
cagtccaatgaacattgaagtgagcgaactgttcgcttcggtggcagtactactcaaagaatgagctgctgttaaaaatgcactct
cgttctctcaagtgagtggcagatgagtgctcacgccttgcacttcgctgcccgtgtcatgccctgcgccccaaaatttgaaaaaag
ggatgagattattgggcaatggacgacgtcgtcgctccgggagtcaggaccggcggaaaataagaggcaacacactccgcttctt
agctcttc
pSZ3841/D2555 (CpaiLPAAT1)
SEQ ID NO: 20

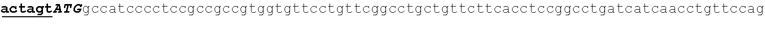

gccttctgcttcgtgctgatctcccccctgtccaagaacgcctaccgccgcatcaaccgcgtgacgccgagctgctgcccctgga
gacctgtggctgttccactggtgcgccggcgccaagctgaagctgttcaccgaccccgagaccttccgcctgatgggcaagga
gcacgccctggtgatcatcaaccacaagatcgagctggactggatggtgggctgggtgctgggccagcacctgggctgcctg
ggctccatcctgtccgtggccaagaagtccaccaagttcctgcccgtgttcggctggtccctgtggttctccggctacctgttcctg
gagcgctcctgggccaaggacaagatcaccctgaagtcccacatcgagtccctgaaggactaccccctgcccttctggctgatc
atcttcgtggagggcacccgcttcacccgcaccaagctgctggccgcccagcagtacgccgcctcctccggcctgcccgtgcccc
gcaacgtgctgatcccccacaccaagggcttcgtgtcctccgtgtcccacatgcgctccttcgtgcccgccatctacgacgtgacc
gtggccttccccaagacctcccccccccccaccatgctgaagctgacgagggccagtccgtggagctgcacgtgcacatcaag
cgccacgccatgaaggacctgcccgagtccgacgacgccgtggcccagtggtgccgcgacaagttcgtggagaaggacgcc
ctgctggacaagcacaactccgaggacaccttctccggccaggaggtgcaccacgtgggccgccccatcaaggccctgctggt
ggtgatctcctgggtggtggtgatcatcttcggcgccctgaagttcctgctgtggtcctccctgctgtcctcctggaagggcaagg
ccactccgtgatcggcctgggcatcgtggccggcatcgtgaccctgctgatgcacatcctgatcctgtcctcccaggccgaggg

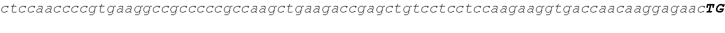

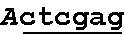

pSZ3842/D2556(CigneaLPAAT1)
SEQ ID NO: 21

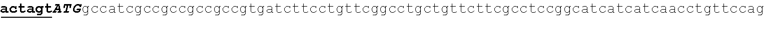

gccctgtgcttcgtgctgatctggcccctgtccaagaacgtgtaccgccgcatcaaccgcgtgacgccgagctgctgctgatgg
acctgctgtgcctgttccactggtgggccggcgccaagatcaagctgacaccgaccccgagaccttccgcctgatgggcatgg
agcacgccctggtgatcatgaaccacaagaccgacctggactggatggtgggctggatcctgggccagcacctgggctgcct
gggctccatcctgtccatcgccaagaagtccaccaagttcatccccgtgctgggctggtccgtgtggactccgagtacctgttcc
tggagcgctcctgggccaaggacaagtccaccctgaagtcccacatggagaagctgaaggactaccccctgcccttctggctg
gtgatcttcgtggagggcacccgcttcacccgcaccaagctgctggccgcccagcagtacgccgcctcctccggcctgcccgtgc
cccgcaacgtgctgatcccccacaccaagggcttcgtgtcctgcgtgtccaacatgcgctccacgtgcccgccgtgtacgacgt
gaccgtggccttccccaagtcctcccccccccccaccatgctgaagctgttcgagggccagtccatcgtgctgcacgtgcacatc
aagcgccacgccctgaaggacctgcccgagtccgacgacgccgtggcccagtggtgccgcgacaagttcgtggagaaggac
gccctgctggacaagcacaacgccgaggacaccttctccggccaggaggtgcaccacatcggccgccccatcaagtccctgct
ggtggtgatcgcctgggtggtggtgatcatcttcggcgccctgaagttcctgcagtggtcctccctgctgtccacctggaagggc
aaggccttctccgtgatcggcctgggcatcgccaccctgctgatgcacatgctgatcctgtcctcccaggccgagcgctccaacc

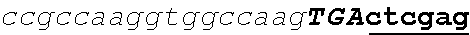

pSZ3844/D2557 (ChookLPAAT1)
SEQ ID NO: 22

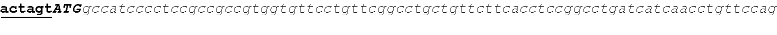

gccttctgcttcgtgctgatctcccccctgtccaagaacgcctaccgccgcatcaaccgcgtgacgccgagctgctgcccctgga
gacctgtggctgttccactggtgcgccggcgccaagctgaagctgttcaccgaccccgagaccttccgcctgatgggcaagga
gcacgccctggtgatcatcaaccacaagatcgagctggactggatggtgggctgggtgctgggccagcacctgggctgcctg
ggctccatcctgtccgtggccaagaagtccaccaagttcctgcccgtgttcggctggtccctgtggttctccgagtacctgttcctg
gagcgctcctgggccaaggacaagatcaccctgaagtcccacatcgagtccctgaaggactaccccctgcccttctggctgatc
atcttcgtggagggcacccgcttcacccgcaccaagctgctggccgcccagcagtacgccgcctcctccggcctgcccgtgcccc
gcaacgtgctgatcccccacaccaagggcttcgtgtcctccgtgtcccacatgcgctccttcgtgcccgccatctacgacgtgacc
gtggccttccccaagacctcccccccccccaccatgctgaagctgacgagggccagtccgtggagctgcacgtgcacatcaag
cgccacgccatgaaggacctgcccgagtccgacgacgccgtggcccagtggtgccgcgacaagttcgtggagaaggacgcc
ctgctggacaagcacaactccgaggacaccttctccggccaggaggtgcaccacgtgggccgccccatcaaggccctgctggt
ggtgatctcctgggtggtggtgatcatcttcggcgccctgaagttcctgctgtggtcctccctgctgtcctcctggaagggcaagg
ccactccgtgatcggcctgggcatcgtggccggcatcgtgaccctgctgatgcacatcctgatcctgtcctcccaggccgaggg

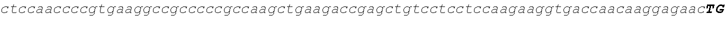

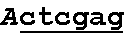

CpauLPAAT1
SEQ ID NO: 23
MAIPAAAVIFLFGLLFFTSGLIINLFQALCFVLVWPLSKNAYRRINRVFAELLLSELLCLFDWWAGAKLKLFTDP
ETFRLMGKEHALVIINHMTELDWMLGWVMGQHLGCLGSILSVAKKSTKFLPVLGWSMWFSEYLYIERSWAKDRTT
LKSHIERLTDYPLPFWMVIFVEGTRFTRTKLLAAQQYAASSGLPVPRNVLIPRTKGFVSCVSHMRSFVPAVYDVT
VAFPKTSPPPTLLNLFEGQSIVLHVHIKRHAMKDLPESDDAVAQWCRDKFVEKDALLDKHNAEDTFSGQEVHRTG
SRPIKSLLVVISWVVVITFGALKFLQWSSWKGKAFSVIGLGIVTLLMHMLILSSQAERSSNPAKVAQAKLKTELS
ISKKATDKEN SEQ ID NO:
CprocL2AAT1
SEQ ID NO: 24
MAIPAAAVIFLFGLIFFASGLIINLFQALCFVLIWPISKNAYRRINRVFAELLLSELLCLFDWWAGAKLKLFTDP
ETFRLMGKEHALVIINHMTELDWMVGWVMGQHFGCLGSILSVAKKSTKFLPVLGWSMWFTEYLYIERSWNKDKST
LKSHIERLKDYPLPFWLVIFAEGTRFTQTKLLAAQQYAASSGLPVPRNVLIPRTKGFVSCVSHMRSFVPAVYDLT
VAFPKTSPPPTLLNLFEGQSVVLHVHIKRHAMKDLPESDDEVAQWCRDKEVEKDALLDKHNAEDTFSGQELQHTG
RRPIKSLLVVISWVVVIAFGALKFLQWSSWKGKAFSVIGLGIVTLLMHMLILSSQAERSKPAKVAQAKLKTELSI
SKTVTDKEN
CprocL2AAT1b
SEQ ID NO: 25
MAIPAAAVIFLFGLIFFASGLIINLFQALCFVLIWPISKNAYRRINRVFAELLLSELLCLFDWWAGAKLKLFTDP
ETFRLMGKEHALVIINHMTELDWMVGWVMGQHFGCLGSILSVAKKSTKFLPVLGWSMWFTEYLYIERSWNKDKST
LKSHIERLKDYPLPFWLVIFAEGTRFTQTKLLAAQQYAASSGLPVPRNVLIPRTKGFVSCVSHMRSFVPAVYDLT
VAFPKTSPPPTLLNLFEGQSVVLHVHIKRHAMKDLPESDDEVAQWCRDKEVEK SEQ ID NO:
CprocL2AAT2a
SEQ ID NO: 26
IVNLVQAVCFVLVRPLSKNTYRRINRVVAELLWLELVWLIDWWAGVKIKVFTDHETFHLMGKEHALVICNHKSDI
DWLVGWVLAQRSGCLGSTLAVMKKSSKFLPVIGWSMWFSEYLFLERNWAKDESTLKSGLNRLKDYPLPFWLALFV
EGTRFTRAKLLAAQQYAASSGLPVPRNVLIPRTKGFVSSVSHMRSFVPAIYDVTVAIPKTSPPPTLIRMFKGQSS
VLHVHLKRHVMKDLPESDDAVAQWCRDIFVEKDALLDKHNADDTFSGQELQDTGRPIKSLLVVISWAVLEVFGAV
KFLQWSSLLSSWKGLAFSGIGLGIITLLMHILILFSQSERSTPAKVAPAKAKIEGESSKTEMEKEK
CprocL2AAT2b
SEQ ID NO: 27
IVNLVQAVCFVLVRPLSKNTYRRINRVVAELLWLELVWLIDWWAGVKIKVFTDHETFHLMGKEHALVICNHKSDI
DWLVGWVLAQRSGCLGSTLAVMKKSSKFLPVIGWSMWFSEYLFLERNWAKDESTLKSGLNRLKDYPLPFWLALFV
EGTRFTRAKLLAAQQYAASSGLPVPRNVLIPRTKGFVSSVSHMRSFVPAIYDVTVAIPKTSPPPTLIRMFKGQSS
VLHVHLKRHVMKDLPESDDAVAQWCRDIFVEKDALLDKHNADDTFSGQELQDTGRPIKSLLV
CpaiLPAAT1
SEQ ID NO: 28
MAIPSAAVVFLFGLLFFTSGLIINLFQAFCFVLISPLSKNAYRRINRVFAELLPLEFLWLFHWCAGAKLKLFTDP
ETFRLMGKEHALVIINHKIELDWMVGWVLGQHLGCLGSILSVAKKSTKFLPVFGWSLWFSGYLFLERSWAKDKIT
LKSHIESLKDYPLPFWLIIFVEGTRFTRTKLLAAQQYAASSGLPVPRNVLIPHTKGFVSSVSHMRSFVPAIYDVT
VAFPKTSPPPTMLKLFEGQSVELHVHIKRHAMKDLPESDDAVAQWCRDKEVEKDALLDKHNSEDTFSGQEVHHVG
RPIKALLVVISWVVVIIFGALKFLLWSSLLSSWKGKAFSVIGLGIVAGIVTLLMHILILSSQAEGSNPVKAAPAK
LKTELSSSKKVTNKEN
ChookLPAAT1
SEQ ID NO: 29
MAIPSAAVVFLFGLLFFTSGLIINLFQAFCFVLISPLSKNAYRRINRVFAELLPLEFLWLFHWCAGAKLKLFTDP
ETFRLMGKEHALVIINHKIELDWMVGWVLGQHLGCLGSILSVAKKSTKFLPVFGWSLWFSEYLFLERSWAKDKIT
LKSHIESLKDYPLPFWLIIFVEGTRFTRTKLLAAQQYAASSGLPVPRNVLIPHTKGFVSSVSHMRSFVPAIYDVT
VAFPKTSPPPTMLKLFEGQSVELHVHIKRHAMKDLPESDDAVAQWCRDKEVEKDALLDKHNSEDTFSGQEVHHVG
RPIKALLVVISWVVVIIFGALKFLLWSSLLSSWKGKAFSVIGLGIVAGIVTLLMHILILSSQAEGSNPVKAAPAK
LKTELSSSKKVTNKEN
ChookL2AAT2a
SEQ ID NO: 30
LSLLFFVSGLIVNLVQAVCFVLIRPLSKNTYRRINRVVAELLWLELVWLIDWWAGVKIKVFTDHETFNLMGKEHA
LVVCNHKSDIDWLVGWVLAQRSGCLGSTLAVMKKSSKFLPVIGWSMWFSEYLFLERSWAKDESTLKSGLKRLKDY
PLPFWLALFVEGTRFTQAKLLAAQQYAASSGLPVPRNVLIPRTKGFVSSVSHMRSFVPAIYDVTVAIPKTSVPPT
MLRIFKGQSSVLHVHLKRHLMKDLPESDDAVAQWCRDIFVEKDALLDKHNAEDTFSGQELQDIGRPIKSLLVVIS
WAVLVIFGAVKFLQWSSLLSSWKGLAFSGIGLGIVTLLMHILILFSQSERSTPAKVAPAKPKNEGESSKTEMEKE
H
ChookL2AAT2b
SEQ ID NO: 31
QIKVFTDHETFNLMGKEHALVVCNHKSDIDWLVGWVLAQWSGCLGSTLAVMKKSSKFLPVIGWSMWFSEYLFLER
SWAKDESTLKSGLKRLKDYPLPFWLALFVEGTRFTQAKLLAAQQYAASSGLPVPRNVLIPRTKGFVSSVSHMRSF
VPAIYDVTVAIPKTSVPPTMLRIFKGQSSVLHVHLKRHLMKDLPESDDAVAQWCRDIFVEKDALLDKHNAEDTFS
GQELQDIGRPIKSLLVVISWAVLVIFGAVKFLQWSSLLSSWKGLAFSGIGLGIVTLLMHILILFSQSERSTPAKV
APAKLKKEGESSKPETDKQN
ChookL2AAT3a
SEQ ID NO: 32
LSLLFFVSGLIVNLVQAVCFVLIRPLLKNTYRRINRVVAELLWLELVWLIDWWAGIKIKVFTDHETFHLMGKEHA
LVICNHKSDIDWLVGWVLAQRSGCLGSTLAVMKKSSKFLPVIGWSMWFSEYLFLERNWAKDESTLKSGLNRLKDY
PLPFWLALFVEGTRFTRAKLLAAQQYAASSGLPVPRNVLIPRTKGFVSSVSQMRSFVPAIYDVTVAIPKTSPPPT
LLRMFKGQSSVLHVHLKRHLMNDLPESDDAVAQWCRDIFVEKDALLDKHNAEDTFSGQELQDTGRPIKSLLVVIS
WATLVVFGAVKFLQWSSLLSSWKGLAFSGIGLGIITLLMHILILFSQSERSTPAKVAPAKPKNEGESSKTEMEKE
H
ChookL2AAT3b
SEQ ID NO: 33
LSLLFFVSGLIVNLVQAVCFVLIRPLLKNTYRRINRVVAELLWLELVWLIDWWAGIKIKVFTDHETFHLMGKEHA
LVICNHKSDIDWLVGWVLAQRSGCLGSTLAVMKKSSKFLPVIGWSMWFSEYLFLERNWAKDESTLKSGLNRLKDY
PLPFWLALFVEGTRFTRAKLLAAQQYAASSGLPVPRNVLIPRTKGFVSSVSQMRSFVPAIYDVTVAIPKTSPPPT
LLRMFKGQSSVLHVHLKRHLMNDLPESDDAVAQWCRDIFVEKDALLDKHNAEDTFSGQELQDIGRPIKSLLVVIS
WAVLEIFGAVKFLQWSSLLSSWKGLAFSGIGLGIVTLLMHILILFSQSERSTPAKVAPAKPKKEGESSKPETDKE
N
CigneaLPAAT1
SEQ ID NO: 34
MAIAAAAVIFLFGLLFFASGIIINLFQALCFVLIWPLSKNVYRRINRVFAELLLMDLLCLFHWWAGAKIKLFTDP
ETFRLMGMEHALVIMNHKTDLDWMVGWILGQHLGCLGSILSIAKKSTKFIPVLGWSVWFSEYLFLERSWAKDKST
LKSHMEKLKDYPLPFWLVIFVEGTRFTRTKLLAAQQYAASSGLPVPRNVLIPHTKGFVSCVSNMRSFVPAVYDVT
VAFPKSSPPPTMLKLFEGQSIVLHVHIKRHALKDLPESDDAVAQWCRDKEVEKDALLDKHNAEDTFSGQEVHHIG
RPIKSLLVVIAWVVVIIFGALKFLQWSSLLSTWKGKAFSVIGLGIATLLMHMLILSSQAERSNPAKVAK
CigneaLPAAT2
SEQ ID NO: 35
MAIAAAAVIFLFGLLFFASGIIINLFQALCFVLIWPLSKNVYRRINRVFAELLLMDLLCLFHWWAGAKIKLFTDP
ETFRLMGMEHALVIMNHKTDLDWMVGWILGQHLGCLGSILSIAKKSTKFIPVLGWSVWFSEYLFLERSWAKDEST
LKSGLNRLKDYPLPFWLALFVEGTRFTRAKLLAAQQYAASSGLPVPKNVLIPRTKGFVSSVSHMRSFVPAIYDVT
VAIPKTSAPPTLLRMFKGQSSVLHVHLKRHLMKDLPESDDAVAQWCRDIFVEKDALLDKHNAEDTFSGQELHDIG
RPVKSLLVVISWAMLVVFGAVKFLQWSSLLSSWKGLAFSGIGLGIITLLMHILILFSQSERSTPAKVAPAKQKNN
EGESSKTEMEKEH
DcLPAAT1
SEQ ID NO: 36
SGLVVNLIQAFFEVLVRPFSKNAYRKINRVVAELLWLELIWLIDWWAGVKIQLYTDPETFKLMGKEHALVICNHK
SDIDWLVGWILAQRSGCLGSALAVMKKSSKFLPVIGWSMWFSEYLFLERSWAKDENTLKSGFQRLRDFPHAFWLA
LEVEGTRFTQAKLLAAQEYASSMGLPAPRNVLIPRTKGFVTAVTHMRPFVPAVYDVTLAIPKTSPPPTMLRLFKG
QSSVVHIHLKRHLMSDLPKSDDSVAQWCKDAFVVKDNLLDKHKENDSFGDGVLQDTGRPLNSLVVVISWACLLIF
GALKFFQWSSILSSWKGLAFSAVGLGIVTVLMQILIQFSQSERSNRPMPSKHAK
DcLPAAT2
SEQ ID NO: 37
MAIPTAAYVVPLGAIFFFSGLLVNLIQAFFFITVWPLSKKTYIRINKVIVELLWLEFVWLADWWAGLKIEVYADA
ETFQLMGKEHALVICNHKSDIDWLVGWILAQRAGCLGSSFAVTKKSARYLPVVGWSIWFSGAIFLERSWEKDENT
LKAGFQRLREFPCAFWLGLFVEGTRFTQAKLLAAQEYASTMGLPFPRNVLIPRTKGFIAAVNHMREFVPAIYDLT
FAFPKDSPPPTMLRLLKGQPSVVHVHIKRHLMKDLPEKNEAVAQWCKDVFLVKDKLLDKHKDDGSFGDGELHEIG
RPLKSLVVVTTWACLLILGTLKFLLWSSLLSSWKGLIFSATGLAVLTVLMQFLIQSTQSERSNPASLSK
CcrLPAATla
SEQ ID NO: 38
LGLLFFISGLAVNLIQAVCFVFLRPLSKNTYRKINRVLAELLWLQLVWLVDWWAGVKIKVFADRESFNLMGKEHA
LVICNHKSDIDWLVGWVLAQRSGCLGSSLAVMKKSSKFLPVIGWSMWFSEYLFLERSWAKDESTLKEGLRRLKDF
PRPFWLALFVEGTRFTQAKLLAAQEYATSQGLPVPRNVLIPRTKVHVHVKRHLMKELPETDEAVAQWCKDLFVEK
DKLLDKHVAEDTFSDQPLQDIGRPVKPLLVVSSWACLVAYGALKFLQWSSLLSSWKGIAVSAVALAIVTILMQIM
ILFSQSERSIPAKVA
CcrLPAAT1b
SEQ ID NO: 39
LGLLFFISGLAVNLIQAVCFVFLRPLSKNTYRKINRVLAELLWLQLVWLVDWWAGVKIKVFADRESFNLMGKEHA
LVICNHKSDIDWLVGWVLAQRSGCLGSSLAVMKKSSKFLPVIGWSMWFSEYLFLERSWAKDESTLKEGLRRLKDF
PRPFWLALFVEGTRFTQAKLLAAQEYATSQGLPVPRNVLIPRTKGFVSAVSHMRSFVPAVYDMTVAIPKSSPSPT
MLRLFKGQSSVVHVHVKRHLMKELPETDEAVAQWCKDLFVEKDKLLDKHVAEDTFSDQPLQDIGRPVKPLLVVSS
WACLVAYGALKFLQWSSLLSSWKGIAVSAVALAIVTILMQIMILFSQSERSIPTKVA
CcrL2AAT2a
SEQ ID NO: 40
MAIAAAAVVFLFGLLFFTSGLIINLAQAVCFVLIWPLSKNAYRRINRVFAELLLLELLWLFHWRAGAKLKLFADP
ETFRLFGKEHALVICNHRTDLDWMVGWVLGQHFGCLGSILSVAKKSTKFLPVLGWSMWFSEYLFLERSWAKDKST
LKSHTERLKDYPLPFWLGIFVEGTRFTRAKLLAAQQYAASSGLPVPRNVLIPHTKLHVHIKRYAMKDLPESDDAV
AQWCRDIYVEKDAFLDKHNAEDTFSGQEVHHIGRPIKSLLVVISWVVVIIFGALKFLRWSSLLSSWKGKAFSVIG
LGIVTLLVNILILSSQAERSNPAKVAPAKLKTELSPSKKVTNKEN
CcrL2AAT2b
SEQ ID NO: 41
MAIAAAAVVFLFGLLFFTSGLIINLAQAVCFVLIWPLSKNAYRRINRVFAELLLLELLWLFHWRAGAKLKLFADP
ETFRLFGKEHALVICNHRTDLDWMVGWVLGQHFGCLGSILSVAKKSTKFLPVLGWSMWFSEYLFLERSWAKDKST
LKSHTERLKDYPLPFWLGIFVEGTRFTRAKLLAAQQYAASSGLPVPRNVLIPHTKGFVSSMSHMRSFVPAVYDLT
VAFPKTSPPPTLLKLFEGQSVVLHVHIKRYAMKDLPESDDAVAQWCRDIYVEKDAFLDKHNAEDTFSGQEVHHIG
RPIKSLLVVISWVVVIIFGALKFLRWSSLLSSWKGKAFSVIGLGIVTLLVNILILSSQAERSNPAKVAPAKLKTE
LSPSKKVTNKEN
BrLPAATla
SEQ ID NO: 42
AAAVIVPLGILFFISGLVVNLLQAICYVLIRPLSKNTYRKINRVVAETLWLELVWIVDWWAGVKIQVFADNETFN
RMGKEHALVVCNHRSDIDWLVGWILAQRSGCLGSALAVMKKSSKFLPVIGWSMWFSEYLFLERNWAKDESTLKSG
LQRLNDFPRPFWLALFVEGTRFTEAKLKAAQEYAASSELPVPRNVLIPRTKGFVSAVSNMRSFVPAIYDMTVAIP
KTSPPPTMLRLFKGQPSVVHVHIKCHSMKDLPESDDAIAQWCRDQFVAKDALLDKHIAADTFPGQQEQNIGRPIK
SLAVVLSWSCLLILGAMKFLHWSNLFSSWKGIAFSALGLGIITLCMQILIRSSQSERSTPAKVVPAKPKDNHNDS
GSSSQTE
BrLPAAT1b
SEQ ID NO: 43
AAAVIVPLGILFFISGLVVNLLQAVCYVLVRPMSKNTYRKINRVVAETLWLELVWIVDWWAGVKIQVFADDETFN
RMGKEHALVVCNHRSDIDWLVGWILAQRSGCLGSALAVMKKSSKFLPVIGWSMWFSEYLFLERNWAKDESTLKSG
LQRLNDFPRPFWLALFVEGTRFTEAKLKAAQEYAASSELPVPRNVLIPRTKGFVSAVSNMRSFVPAIYDMTVAIP
KTSPPPTMLRLFKGQPSVVHVHIKCHSMKDLPESDDAIAQWCRDQFVAKDALLDKHIAADTFPGQQEQNIGRPIK
SLAVVLSWSCLLILGAMKFLHWSNLFSSWKGIAFSALGLGIITLCMQILIRSSQSERSTPAKVVPAKPKDNHNDS
GSSSQTE
BrLPAAT1c
SEQ ID NO: 44
MAIAAAVIVPLGLLFFISGLLMNLLQAICYVLVRPLSKNTYRKINRVVAETLWLELVWIVDWWAGVKIKVFADNE
TFSRMGKEHALVVCNHRSDIDWLVGWILAQRSGCLGSALAVMKKSSKFLPVIGWSMWFSEYLFLERNWAKDESTL
KSGLQRLNDFPRPFWLALFVEGTRFTEAKLKAAQEYAASSELPVPRNVLIPRTKGFVSAVSNMRSFVPAIYDMTV
AIPKTSPPPTMLRLFKGQPSVVHVHIKCHSMKDLPESDDAIAQWCRDQFVAKDALLDKHIAADTFPGQQEQNIGR
PIKSLAVVLSWSCLLILGAMKFLHWSNLFSSWKGIAFSALGLGIITLCMQILIRSSQSERSTPAKVVPAKPKDNH
NDSGSSSQTE
BjLPAAT1a
SEQ ID NO: 45
INLVVAETLWLELVWIVDWWAGVKIQVFADDETFNRMGKEHALVVCNHRSDIDWLVGWILAQRSGCLGSALAVMK
KSSKFLPVIGWSMWFSEYLFLERNWAKDESTLKSGLQRLNDFPRPFWLALFVEGTRFTEAKLKAAQEYAASSELP
VPRNVLIPRTKGFVSAVSNMRSFVPAIYDMTVAIPKTSPPPTMLRLFKGQPSVVHVHIKCHSMKDLPESDDAIAQ
WCRDQFVAKDALLDKHIAADTFPGQKEQNIGRPIKSLAVSLIKTFPWLHPHQLTNIFVLFQVVVSWACLLTLGAM
KFLHWSNLFSSWKGIALSAFGLGIITLCMQILIRSSQSERSTPAKVAPAKPK
BjLPAAT1b
SEQ ID NO: 46
INLVVAETLWLELVWIVDWWAGVKIQVFADDETFNRMGKEHALVVCNHRSDIDWLVGWILAQRSGCLGSALAVMK
KSSKFLPVIGWSMWFSEYLFLERNWAKDESTLKSGLQRLNDFPRPFWLALFVEGTRFTEAKLKAAQEYAASSELP
VPRNVLIPRTKGFVSAVSNMRSFVPAIYDMTVAIPKTSPPPTMLRLFKGQPSVVHVHIKCHSMKDLPEPEDEIAQ
WCRDQFVAKDALLDKHIAADTFPGQKEQNIGRPIKSLAVVVSWACLLTLGAMKFLHWSNLFSSWKGIALSAFGLG
IITLCMQILIRSSQSERSTPAKVAPAKPK
BjLPAAT1c
SEQ ID NO: 47
INLVVAETLWLELVWIVDWWAGVKIQVFADDETFNRMGKEHALVVCNHRSDIDWLVGWILAQRSGCLGSALAVMK
KSSKFLPVIGWSMWFSEYLFLERNWAKDESTLKSGLQRLNDFPRPFWLALFVEGTRFTEAKLKAAQEYAASSELP
VPRNVLIPRTKGFVSAVSNMRSFVPAIYDMTVAIPKTSPPPTMLRLFKGQPSVVHVHIKCHSMKDLPESDDAIAQ
WCRDQFVAKDALLDKHIAADTFPGQQEQNIGRPIKSLAVVLSWSCLLILGAMKFLHWSNLFSSWKGIAFSALGLG
IITLCMQILIRSSQSERSTPAKVVPAKPKDNHNDSGSSSQTE
BjLPAAT1d
SEQ ID NO: 48
INLVVAETLWLELVWIVDWWAGVKIQVFADDETFNRMGKEHALVVCNHRSDIDWLVGWILAQRSGCLGSALAVMK
KSSKFLPVIGWSMWFSEYLFLERNWAKDESTLKSGLQRLNDFPRPFWLALFVEGTRFTEAKLKAAQEYAASSELP
VPRNVLIPRTKGFVSAVSNMRSFVPAIYDMTVAIPKTSPPPTMLRLFKGQPSVVHVHIKCHSMKDLPESDDAIAQ
WCRDQFVAKDALLDKHIAADTFPGQQEQNIGRPIKSLAVSLS
CcLPAAT1a
SEQ ID NO: 49
MAIGVAAIVVPLGLLFILSGLMVNLIQAICFILVRPLSKNMYRRVNRVVVELLWLELIWLIDWWGGVKVDVYADS
ETFQSLGKEHALVVSNHRSDIDWLVGWVLAQRSGCLGSTLAVMKKSSKFLPVIGWSMWFSEYVFLERSWAKDEST
LKSGLRRLKDFPRPFWLALFVEGTRFTQAKLLAAREYAASTGLPIPRNVLIPRTKGFVSAVSNMRSFVPAIYDVT
VAIPKTQPSPTMLRIFNRQPSVVHVHIKRHSMNQLPQTDEGVGQWCKDIFVAKDALLDRHLAE
CcLPAAT1b
SEQ ID NO: 50
MAIGVAAIVVPLGLLFILSGLMVNLIQAICFILVRPLSKNMYRRVNRVVVELLWLELIWLIDWWGGVKVDVYADS
ETFQSLGKEHALVVSNHRSDIDWLVGWVLAQRSGCLGSTLAVMKKSSKFLPVIGWSMWFSEYVFLERSWAKDEST
LKSGLRRLKDFPRPFWLALFVEGTRFTQAKLLAAREYAASTGLPIPRNVLIPRTKGFVSAVSNMRSFVPAIYDVT
VAIPKTQPSPTMLRIFNRQPSVVHVHIKRHSMNQLPQTDEGVAQWCKDIFVAKDALLDRHLAEGKFDEKEFKRIR
RPIKSLLVISSWSFLLMFGVFKFLKWSALLSTWKGVAVSTTVLLLVTVVMYMFILFSQSERSSPRKVAPSGPENG
UcLPAAT1a
SEQ ID NO: 51
MAIGVAAIVVPLGLLFILSGLIINLIQAICFILVRPLSKNMYRKVNRVVVELLWLELIWLIDWWGGVKVDVYADS
ETFQSLGKEHALVVSNHRSDIDWLVGWVLAQRSGCLGSTLAVMKKSSKFLPVIGWSMWFSEYVFLERSWAKDEST
LKSGLQRLKDFPRPFWLALFVEGTRFTQAKLLAAQEYAASTGLPIPRNVLIPRTKGEVSAVSNMRSFVPAIYDVT
VAIPKTQPSPTMLRIFNRQPSVVHVHIKRHSMNQLPQTDEGVAQWCKDIFVAKDALLDRHLAEGKFDEKEFKLIR
RPIKSLLVISSWSFLLMFGVFKFLKWSALLSTWKGVAVSTAVLLLVTVVMYMFILFSQSERSSPRKVAPIGPENG
UcLPAAT1b
SEQ ID NO: 52
MAIGVAAIVVPLGLLFILSGLIINLIQAICFILVRPLSKNMYRKVNRVVVELLWLELIWLIDWWGGVKVDVYADS
ETFQSLGKEHALVVSNHRSDIDWLVGWVLAQRSGCLGSTLAVMKKSSKFLPVIGWSMWFSEYVFLERSWAKDEST
LKSGLQRLKDFPRPFWLALFVEGTRFTQAKLLAAQEYAASTGLPIPRNVLIPRTKGEVSAVSNMRSFVPAIYDVT
VAIPKTQPSPTMLRIFNRQPSVVHVHIKRHSMNQLPQTDEGVAQWCKDIFVAKDALLDRHLAE
LdLPAAT1
SEQ ID NO: 53
SLLFFMSGLVVNFIQAVFYVLVRPISKNTYRRINTLVAELLWLELVWVIDWWAGVKVQLYTDTESFRLMGKEHAL
LICNHRSDIDWLIGWVLAQRCGCLSSSIAVMKKSSKFLPVIGWSMWFSEYLFLERNWAKDENTLKSGLQRLNDFP
KPFWLALFVEGTRFTKAKLLAAQEYAASAGLPVPRNVLIPRTKGFVSAVSNMRSFVPAIYDLTVAIPKTTEQPTM
LRLFRGKSSVVHVHLKRHLMKDLPKTDDGVAQWCKDQFISKDALLDKHVAEDTFSGLEVQDIGRPMKSLVVVVSW
MCLLCLGLVKFLQWSALLSSWKGMMITTFVLGIVTVLMHILIRSSQSEHSTPAK
CaequLPAAT1a
SEQ ID NO: 54
QRSGCLGSTLAVMKKSSKFLPVIGWSMWFSEYLFLERSWAKDESTLKSGLKRLKDYPLPFWLALFVEGTRFTQAK
LLAAQQYAASSGLPVPRNVLIPRTKGEVSSVSHMRSFVPAIYDVTVAIPKMSTPPTMLRIFKGQSSVLHVHLKRH
LMKDLPESDDAVAQWCRDIFVEKDALLDKHNAEDTFSGQELQDIGRPVKSLLVVISWAVLVIFGAVKFLQWSSLL
SSWKGLAFSGIGLGIVTLLMHILILFSQSERSTPAKVAPAKPKKEGESSKTETEKEN
CaequLPAAT1b
SEQ ID NO: 55
DWWAGVKIKVFTDHETLSLMGKEHALVISNHKSDIDWLVGWVLAQRSGCLGSTLAVMKKSSKFLPVIGWSMWFSE
YLFLERSWAKDESTLKSGLKRLKDYPLPFWLALFVEGTRFTQAKLLAAQQYAASSGLPVPRNVLIPRTKGEVSSV
SHMRSFVPAIYDVTVAIPKMSTPPTMLRIFKGQSSVLHVHLKRHLMKDLPESDDAVAQWCRDIFVEKDALLDKHN
AEDTFSGQELQDIGRPVKSLLV
CaequLPAAT1c
SEQ ID NO: 56
DWWAGVKIKVFTDHETLSLMGKEHALVISNHKSDIDWLVGWVLAQRSGCLGSTLAVMKKSSKFLPVIGWSMWFSE
YLFLERSWAKDESTLKSGLKRLKDYPLPFWLALFVEGTRFTQAKLLAAQQYAASSGLPVPRNVLIPRTKGEVSSV
SHMRSFVPAIYDVTVAIPKMSTPPTMLRIFKGQSSVLHVHLKRHLMKDLPESDDAVAQWCRDIFVEKDALLDKHN
AEDTFSGQELQDIGRPVKSLLVVISWAVLVIFGAVKFLQWSSLLSSWKGLAFSGIGLGIVTLLMHILILFSQSER
STPAKVAPAKPKKEGESSKTETEKEN
CaequLPAAT1d
SEQ ID NO: 57
QRSGCLGSTLAVMKKSSKFLPVIGWSMWFSEYLFLERSWAKDESTLKSGLKRLKDYPLPFWLALFVEGTRFTQAK
LLAAQQYAASSGLPVPRNVLIPRTKGEVSSVSHMRSFVPAIYDVTVAIPKMSTPPTMLRIFKGQSSVLHVHLKRH
LMKDLPESDDAVAQWCRDIFVEKDALLDKHNAEDTFSGQELQDIGRPVKSLLV
CglutLPAAT1a
SEQ ID NO: 58
LSLLFFVSGLFVNLVQAVCFVLIRPFSKNTYRRINRVVAELLWLELVWLIDWWAGVKIKVFTDHETLSLMGKEHA
LVISNHKSDIDWLVGWVLAQRSGCLGSTLAVMKKSSKFLPVIGWSMWFSEYLFLERSWAKDESTLKSGLKRLKDY
PLPFWLALFVEGTRFTQAKLLAAQQYAASSGLPVPRNVLIPRTKGFVSSVSHMRSFVPAIYDVTVAIPKMSTPPT
MLRIFKGQSSVLHVHLKRHLMKDLPESDDAVAQWCRDIFVEKDALLDKHNAEDTFSGQELQDIGRPVKSLLVVIS
WAVLVIFGAVKFLQWSSLLSSWKGLAFSGIGLGIVTLLMHILILFSQSERSTPAKVAPAKPKKEGESSKTETEKE
N
CglutLPAAT1b
SEQ ID NO: 59
QAVCFVLIRPFSKNTYRRINRVVAELLWLELVWLIDWWAGVKIKVFTDHETLSLMGKEHALVISNHKSDIDWLVG
WVLAQRSGCLGSTLAVMKKSSKFLPVIGWSMWFSEYLFLERSWAKDESTLKSGLKRLKDYPLPFWLALFVEGTRF
TQAKLLAAQQYAASSGLPVPRNVLIPRTKGFVSSVSHMRSFVPAIYDVTVAIPKMSTPPTMLRIFKGQSSVLHVH
LKRHLMKDLPESDDAVAQWCRDIFVEKDALLDKHNAEDTFSGQELQDIGRPVKSLLVVISWAVLVIFGAVKFLQW
SSLLSSWKGLAFSGIGLGIVTLLMHILILFSQSERSTPAKVAPAKPKKEGESSKTETEKEN
CprLPAAT1
SEQ ID NO: 60
MAIAAAAVVFLFGLLFFTSGLIINLAQAVCFVLIWPLSKNAYRRINRVFAELLLLELLWLFHWRAGAKLKLFADP
ETFRLFGKEHALVICNHRTDLDWMVGWVLGQHFGCLGSILSVAKKSTKFLPVLGWSMWFSEYLFLERSWAKDKST
LKSHTERLKDYPLPFWLGIFVEGTRFTRAKLLAAQQYAASSGLPVPRNVLIPHTKGFVSSMSHMRSFVPAVYDLT
VAFPKTSPPPTLLKLFEGQSVVLHVHIKRYAMKDLPESDDAVAQWCRDIYVEKDAFLDKHNAEDTFSGQEVHHIG
RPIKSLLVVISWVVVIIFGALKFLRWSSLLSSWKGKAFSVIGLGIVTLLVNILILSSQAERSNPAKVVPAKLKTE
LSPSKKVTNKEN
ChsLPAAT1
SEQ ID NO: 61
MAIPSAAVVFLFGLLFFASGLIINLVQAVCFVLIWPLSKNTCRRINIVFQDMLLSELLWLFHWRAGAKLKFFTDP
ETYRHMGKEHALVITNHRTDLDWMIGWVLGEHLGCLGSILSVVKKSTKFLPVLGWSMWFSEYLFLERNWAKDKST
FKSHIERLEDFPQPFWFGIFVEGTRFTRAKLLAAQQYAASSGLPVPRNVLIPHTKGFVSSVSHMRSFVPAVYETT
MTFPKTSPPPTLLKLFEGQPLVLHIHMKRHAMKDIPESDDAVAQWCRDKFVEKDALLDKHNAEDTFGGLEVHIGR
SIKSLMVVICWVVVIIFGALKFLQWSSLLSSWKGIAFIGIGLGIVNLLVHVLILSSQAERSAPTKVAPAKLKTKL
LSSKKITNKEN
ChsLPAAT2
SEQ ID NO: 62
MAIPSAAVVFLFGLLFFASGLIINLVQAVCFVLIWPLSKNTCRRINIVFQDMLLSELLWLFHWRAGAKLKFFTDP
ETYRHMGKEHALVITNHRTDLDWMIGWVLGEHLGCLGSILSVVKKSTKFLPVLGWSMWFSEYLFLERNWAKDKST
FKSHIERLEDFPQPFWFGIFVEGTRFTRAKLLAAQQYAASSGLPVPRNVLIPRTKGFVSSVSHMRSFVPAIYDVT
VAIPKTSPPPTMLRMFKGQSSVLHVHLKRHLMKDLPESDDAVAQWCRDIFVEKDALLDKHNAEDTFSGQELQDIG
RPIKSLVVVISWAALVVFGAVKFLQWSSLLSSWKGLAFSGIGLGIITLLMHILILFSQSERSTPAKVAPAKPKRE
GESSKTEMDKEN
CcalcLPAAT1a
SEQ ID NO: 63
MAIPAAAVVFLFGLLFFPSGLIINLFQAVCFVLIWPFSRNTCRRINIVFQEMLLSELLWLFHWRAGAKLKLFADP
ETYRHMGKEHALLITNHRTDLDWMIGWALGQHLGCLGSILSVVKKSTKFLPSHIERLEDFPQPFWMAIFVEGTRF
TRAKLLAAQQYAASSGLPVPRNVLIPRTKGFVSCVSHMRSFVPAVYETTMTFPKTSPPPTLLKLFEGQPIVLHVH
MKRHAMKDIPESDEAVAQWCRDKFVEKDSLLDKHNAGDTFSCQEIHIGRPIKSLMVVISWVVVIIFGALKFLQWS
SLLSSWKGIAFSGIGLGIVTLLVHILILSSQAERSTPAKVAPAKLKTELSSSTKVTNKEN
CcalcLPAAT1b
SEQ ID NO: 64
MAIPAAAVVFLFGLLFFPSGLIINLFQAVCFVLIWPFSRNTCRRINIVFQEMLLSELLWLFHWRAGAKLKLFADP
ETYRHMGKEHALLITNHRTDLDWMIGWALGQHLGCLGSILSVVKKSTKFLPVLGWSMWFSEYLFLERNWAKDKST
FKSHIERLEDFPQPFWMAIFVEGTRFTRAKLLAAQQYAASSGLPVPRNVLIPRTKGFVSCVSHMRSFVPAVYETT
MTFPKTSPPPTLLKLFEGQPIVLHVHMKRHAMKDIPESDEAVAQWCRDKFVEKDSLLDKHNAGDTFSCQEIHIGR
PIKSLMVVISWVVVIIFGALKFLQWSSLLSSWKGIAFSGIGLGIVTLLVHILILSSQAERSTPAKVAPAKLKTEL
SSSTKVTNKEN
Cca1cLPAAT2
SEQ ID NO: 65
LSLLFFVSGLIVNLVQAVCFVLIRPLSKNTYRRINRVVAELLWLELVWLIDWWAGVKIKVFTDHETFRLMGTEHA
LVISNHKSDIDWLVGWVLAQRSGCLGSTLAVMKKSSKFLPVIGWSMWFSEYLFLERSWAKDESTLKSGLNRLKDY
PLPFWLALFVEGTRFTRAKLLAAQQYAASSGLPVPRNVLIPRTKGFVSSVSHMRSFVPAIYDVTVAIPKTSPPPT
MLRMFKGQSSVLHVHLKRHLMKDLPESDDAVAQWCRDIFVEKDALLDKHNAEDTFSGQELQDIGRPIKSLVVVIS
WAALVVFGAVKFLQWSSLLSSWKGLAFSGIALGIITLLMHILILFSQSERSTPAKVAPAKPKKEGESSKTETDKE
N
ChtLPAAT1a
SEQ ID NO: 66
MAIPAAAVIFLFSILFFASGLIINLVQAVCFVLIWPLSKNTCRRINLVFQEMLLSELLGLFHWRAGAKLKLYTDP
ETYPLLGKEHALLMINHRTDLDWMIGWVLGQHLGCLGSILSVVKKSTKFLPVLGWSMWFSEYLFLERNWAKDKST
FKSHIERLEDFPQPFWMAIFVEGTRFTRAKLLAAQQYAASSGLPVPRNVLIPHTKGFVSTVSHMRSFVPAVYDTT
LTFPKTSPPPTLLNLFAGQPIVLHIHIKRHAMKDIPESDDAVAQWCRDKFVEKDALLDKHNAEDAFSDQEFPISR
SIKSLMVVISWVMVIIFGALKFLQWSSLLSSWKGKAFSVIAVGIVTLLMHMSILSSQAERSNPAKVALPKLKTEL
PSSKKVLNKEN
ChtLPAAT1b
SEQ ID NO: 67
MAIPAAAVIFLFSILFFASGLIINLVQAVCFVLIWPLSKNTCRRINLVFQEMLLSELLGLFHWRAGAKLKLYTDP
ETYPLLGKEHALLMINHRTDLDWMIGWVLGQHLGCLGSILSVVKKSTKFLPVLGWSMWFSEYLFLERNWAKDKST
FKSHIERLEDFPQPFWMAIFVEGTRFTRAKLLAAQQYAASSGLPVPRNVLIPHTKGFVSTVSHMRSFVPAVYDTT
LTFPKTSPPPTLLNLFAGQPIVLHIHIKRHAMKDIPESDDAVAQWCRDKFVEKDALLDKHNAEDAFSDQEFPISR
SIKSLMVVISWVMVIIFGALKFLQWSSLLSSWKGIAFSGIGLGIVTLLMHILILSSQAERSTPAKVAQAKVKTEL
PSSTKVTNKGN
CwLPAAT1
SEQ ID NO: 68
MAIPAAAVIFLFGILFFASGLIINLVQAVCFVLIWPLSKNTCRRINLVFQEMLLSELLWLFHWRAGAELKLFTDP
ETYRLLGKEHALVMTNHRTDLDWMIGWVTGQHLGCLGSILSIAKKSTKFLPVLGWSMWFSEYLFLERNWAKDKST
FKSHIERLEDFPQPFWMAIFVEGTRFTRAKLLAAQQYAASSGLPVPRNVLIPHTKGFVSSVCHMRSFVPAVYDTT
LTFPKNSPPPTLLNLFAGQPIVLHIHIKRHAMKDMPKSDDAVAQWCRDKFVKKDALLDKHNTEDTFSDQEFPIGR
PIKSLMVVISWVVVIIFGTLKFLQWSSLLSSWKGIAFSGIGLGIVTLLVHILILSSQAERSTPPKVAPAKLKTEL
SSTTKVINKGN
CwLPAAT2b
SEQ ID NO: 69
LGLLFFVSGLIVNLVQAVCFVLIRPLSKNTYRRLNRVVAELLWLELVWLIDWWAGVKIKVFTDHETFHLMGKEHA
LVICNHKSDIDWLVGWVLAQRSGCLGSTLAVMKKSSKFLPVIGWSMWFSEYLFLERSWAKDESTLKSGLNRLKDY
PLPFWLALFVEGTRFTRAKLLAAQQYAASSGLPVPRNVLIPRTKGFVSSVSHMRSFVPAIYDVTVAIPKTSPPPT
MLRMFKGQSSVDALLDKHNADDTFSGQELHDIGRPIKSLLVVISWAVLVVFGAVKFLQWSSLLSSWKGIAFSGIG
LGIVTLLVHILILSSQAERSTSAKVAQAKVKTELSSSKKVKNKGN
CwLPAAT2a
SEQ ID NO: 70
LGLLFFVSGLIVNLVQAVCFVLIRPLSKNTYRRLNRVVAELLWLELVWLIDWWAGVKIKVFTDHETFHLMGKEHA
LVICNHKSDIDWLVGWVLAQRSGCLGSTLAVMKKSSKFLPVIGWSMWFSEYLFLERSWAKDESTLKSGLNRLKDY
PLPFWLALFVEGTRFTRAKLLAAQQYAASSGLPVPRNVLIPRTKGFVSSVSHMRSFVPAIYDVTVAIPKTSPPPT
MLRMFKGQSSVLHVHLKRHLMKDLPESDDAVAQWCRDIFVEKDVLLDKHNAEDTFSGQELQDIGRPVKSLLVVIS
WTLLVIFGAVKFLQWSSLLSSWKGLAFSGIGLGIVTLLMHILILFSQSERSTPAKVAPAKPKKEGESSKMETDKE
N
CgLPAAT1a
SEQ ID NO: 71
LAGWMGSSSGCLGSTLAVMKKSSKFLPVIGWSMWFSEYLFLERSWAKDESTLKSGLNRLKDYPLPFWLALFVEGT
RFTRAKLLAAQQYAASLGLPVPRNVLIPRTKGFVSSVSHMRSFVPAIYDVTVAIPKTSPPPTMIRMFKGQSSVLH
VHLKRHVMKDLPESDDAVAQWCRDIFVEKDALLDKHNAEDTFSGQELQDTGRPIKSLLVVISWAVLEVFGAVKFL
QWSSLLSSWKGLAFSGIGLGIITLLMHILILFSQSERSTPAKVAPAKPKNEGESSKAEMEKEK
CgLPAAT1b
SEQ ID NO: 72
LAGWMGSSSGCLGSTLAVMKKSSKFLPVIGWSMWFSEYLFLERSWAKDESTLKSGLNRLKDYPLPFWLALFVEGT
RFTRAKLLAAQQYAASLGLPVPRNVLIPRTKGFVSSVSHMRSFVPAIYDVTVAIPKTSPPPTMIRMFKGQSSVLH
VHLKRHVMKDLPESDDAVAQWCRDIFVEKDALLDKHNAEDTFSGQELQDTGRPIKSLLVRCFLVLSLIYLNGIML
KLRGPCLQVVISWAVLEVFGAVKFLQWSSLLSSWKGLAFSGIGLGIITLLMHILILFSQSERSTPAKVAPAKPKN
EGESSKAEMEKEK
CgLPAAT1c
SEQ ID NO: 73
LAGWMGSSSGCLGSTLAVMKKSSKFLPVIGWSMWFSEYLFLERSWAKDESTLKSGLNRLKDYPLPFWLALFVEGT
RFTRAKLLAAQQYAASLGLPVPRNVLIPRTKGFVSSVSHMRSFVPAIYDVTVAIPKTSPPPTMIRMFKGQSSVLH
VHLKRHVMKDLPESDDAVAQWCRDIFVEKDALLDKHNAEDTFSGQELQDTGRPIKSLLVVTSWAVLVISGAVKFL
QWSSLLSSWKGLAFSGIGLGIVTLLMHILILFSQSERSTPAKVAPAKPKKEGESSKTEKDKEN
CpalLPAAT1
SEQ ID NO: 74
LGLLFFVSGLIVNLVQAVCFVLIRPLSKNTYRRINRVVAELLWLELVWLIDWWAGVKIKVFTDHETLSLMGKEHA
LVICNHKSDIDWLVGWVLAQRSGCLGSTLAVMKKSSKFLPVIGWSMWFSEYLFLERSWAKDENTLKSGLNRLKDY
PLPFWLALFVEGTRFTRAKLLAAQQYATSSGLPVPRNVLIPRTKGFVSSVSHMRSFVPAIYDVTVAIPKTSPPPT
MLRMFKGQSSVLHVHLKRHLMKDLPESDDAVAQWCRDIFVEKDALLDKHNAEDTFSGQELQDTGRPIKSLLVVIS
WAVLVIFGAVKFLQWSSLLSSWKGLAFSGVGLGIITLLMHILILFSQSERSTPAKVAPAKPKKDGESSKTEIEKE
N
CaLPAAT1
SEQ ID NO: 75
MAIAAAAVIVPVSLLFFVSGLIVNLVQAVCFVLIRPLFKNTYRRINRVVAELLWLELVWLIDWWAGVKIKVFTDH
ETFHLMGKEHALVICNHKSDIDWLVGWVLAQRSGCLGSTLAVMKKSSKFLPVIGWSMWFSEYLFLERNWAKDEST
LKSGLNRLKDYPLPFWLALFVEGTRFTRAKLLAAQQYAASSGLPVPRNVLIPRTKGFVSSVSHMRSFVPAIYDVT
VAIPKTSPPPTLLRMFKGQSSVLHVHLKRHQMNDLPESDDAVAQWCRDIFVEKDALLDKHNAEDTFSGQELQDTG
RPIKSLLIVISWAVLVVFGAVKFLQWSSLLSSWKGLAFSGIGLGVITLLMHILILFSQSERSTPAKVAPAKPKIE
GESSKTEMEKEH
CaLPAAT3
SEQ ID NO: 76
MTIASAAVVFLFGILLFTSGLIINLFQAFCSVLVWPLSKNAYRRINRVFAEFLPLEFLWLFHWWAGAKLKLFTDP
ETFRLMGKEHALVIINHKIELDWMVGWVLGQHLGCLGSILSVAKKSTKFLPVFGWSLWFSEYLFLERNWAKDKKT
LKSHIERLKDYPLPFWLIIFVEGTRFTRTKLLAAQQYAASAGLPVPRNVLIPHTKGFVSSVSHMRSFVPAIYDVT
VAFPKTSPPPTMLKLFEGHFVELHVHIKRHAMKDLPESEDAVAQWCRDKEVEKDALLDKHNAEDTFSGQEVHHVG
RPIKSLLVVISWVVVIIFGALKFLQWSSLLSSWKGIAFSVIGLGTVALLMQILILSSQAERSIPAKETPANLKTE
LSSSKKVTNKEN
SalL2AAT1
SEQ ID NO: 77
MAIGAAAIVVPLGLLFMLSGLMVNLIQAICFILVRPLSKNMYRRVNRVVVELLWLELIWLIDWWGGVKVDVYADS
ETFQSLGKEHALVVSNHKSDIDWLVGWVLAQRSGCLGSTLAVMKKSSKFLPVIGWSMWFSEYVFLERSWAKDEST
LKSGLQRLKDFPRPFWLALFVEGTRFTQAKLLAAQEYAASTGLPIPRNVLIPRTKGFVSAVSNMRSFVPAIYDVT
VAIPKTQPSPTMLRIFNRQPSVVHVRIKRHSMNQLPPTDEGVAQWCKDIFVAKDALLDRHLAEGKFDEKEFKRIR
RPIKSLLVISSWSFLLLFGVFKFLKWSALLSTWKGVAVSTAVLLLVTVVMYMFILFSQSERSSPRKVAPSGPENG
C1eptL2AAT1
SEQ ID NO: 78
MAIPAAVVIFLFGLLFFSSGLIINLFQALCFVLIWPLSKNAYRRINRVFAELLLSELLCLFDWWAGAKLKLFTDP
ETFRLMGKEHALVIINHMTELDWMVGWVMGQHFGCLGSILSVAKKSTKFLPVLGWSMWFTEYLYIERSWDKDKST
LKSHIERLKDYPLPFWLVIFAEGTRFTRTKLLAAQQYAASSGLPVPRNVLIPRTKGFVSCVNHMRSFVPAVYDLT
VAFPKTSPPPTLLNLFEGQSVVLHVHIKRHAMKDLPESDDAVAQWCRDKFVEKDALLDKHNAEDTFSSQEVHHTG
SRPIKSLLVVISWVVVITFGALKFLQWSSWKGKAFSVIGLGIVTLLMHMLILSSQAERSKPAKVTQAKLKTELSI
SKKVTDKEN
ClopLPAAT1
SEQ ID NO: 79
MAIAAAAVIFLFGLLFFASGLIINLFQALCFVLIRPLSKNAYRRINRVFAELLLSELLCLFDWWAGAKLKLFTDP
ETLRLMGKEHALIIINHMTELDWMVGWVMGQHFGCLGSIISVAKKSTKFLPVLGWSMWFSEYLYLERSWAKDKST
LKSHIERLKDYPLPFWLVIFVEGTRFTRTKLLAAQEYAASSGLPVPRNVLIPRTKGFVSCVNHMRSFVPAVYDVT
VAFPKTSPQPTLLNLFEGRSIVLHVHIKRHAMKDLPESDDAVAQWCRDKFVEKDALLDKHNAEDTFSGQEVHHTG
RRPIKSLLVVMSWVVVTTFGALKFLQWSSWKGKAFSVIGLGIVTLLMHVLILSSQAERSNPAKVVQAELNTELSI
SKKVTNKGN
CcrasLPAAT1a
SEQ ID NO: 80
MAIPAAAVIFLFGLIFFASGLIINLFQALCFVLIWPLWKNAYRRINRVFAELLLSELLCLFDWWAGAKLKLFTDP
ETFRLMGKEHALVIINHMTELDWMVGWVMGQHFGCLGSILSVAKKSTKFLPVLGWSMWFTEYLYIERSWDKDKST
LKSHIERLKDYPLPFWLVIFAEGTRFTRTKLLAAQQYAASSGLPVPRNVLIPRTKGFVSSVSHMRSFVPAIYDVT
VAIPKTSPPPTLIRMFKGQSSVLHVHLKRHVMKDLPESDDAVAQWCRDIFVEKDALLDKHNAEDTFSGQELQDTG
RPIKSLLVVISWAVLEVFGAVKFLQWSSLLSSWKGLAFSGIGLGIITLLMHILILFSQSERSTPAKVAPAKAK
CcrasLPAAT1b
SEQ ID NO: 81
MAIPAAAVIFLFGLIFFASGLIINLFQALCFVLIWPLWKNAYRRINRVFAELLLSELLCLFDWWAGAKLKLFTDP
ETFRLMGKEHALVIINHMTELDWMVGWVMGQHFGCLGSILSVAKKSTKFLPVLGWSMWFTEYLYIERSWDKDKST
LKSHIERLKDYPLPFWLVIFAEGTRFTRTKLLAAQQYAASSGLPVPRNVLIPRTKGFVSSVSHMRSFVPAIYDVT
VAIPKTSPPPTLIRMFKGQSSVLHVHLKRHVMKDLPESDDAVAQWCRDIFVEKDALLDKHNAEDTFSGQELQDTG
RPIKSLLVRCFLVLSLIYLNGIILKLCGLCLQVVISWAVLEVFGAVKFLQWSSLLSSWKGLAFSGIGLGIITLLM
HILILFSQSERSTPAKVAPAKAKSEQ ID NO:
CcrasLPAAT1c
SEQ ID NO: 82
MAIPAAAVIFLFGLIFFASGLIINLFQALCFVLIWPLWKNAYRRINRVFAELLLSELLCLFDWWAGAKLKLFTDP
ETFRLMGKEHALVIINHMTELDWMVGWVMGQHFGCLGSILSVAKKSTKFLPVLGWSMWFTEYLYIERSWDKDKST
LKSHIERLKDYPLPFWLVIFAEGTRFTRTKLLAAQQYAASSGLPVPRNVLIPRTKGFVSSVSHMRSFVPAIYDVT
VAIPKTSPPPTLIRMFKGQSSVLHVHLKRHVMKDLPESDDAVAQWCRDIFVEKDALLDKHNAEDTFSGQELQDTG
RPIKSLLVVISWAVLEVFGAVKFLQWSSLLSSWKGLAFSGIGLGIITLLMHILILFSQSERSTPAKVAPAKAKME
GESSKTEMEMEK
CcrasLPAAT1d
SEQ ID NO: 83
MAIPAAAVIFLFGLIFFASGLIINLFQALCFVLIWPLWKNAYRRINRVFAELLLSELLCLFDWWAGAKLKLFTDP
ETFRLMGKEHALVIINHMTELDWMVGWVMGQHFGCLGSILSVAKKSTKFLPVLGWSMWFTEYLYIERSWDKDKST
LKSHIERLKDYPLPFWLVIFAEGTRFTRTKLLAAQQYAASSGLPVPRNVLIPRTKGFVSSVSHMRSFVPAIYDVT
VAIPKTSPPPTLIRMFKGQSSVLHVHLKRHVMKDLPESDDAVAQWCRDIFVEKDALLDKHNAEDTFSGQELQDTG
RPIKSLLVRCFLVLSLIYLNGIILKLCGLCLQVVISWAVLEVFGAVKFLQWSSLLSSWKGLAFSGIGLGIITLLM
HILILFSQSERSTPAKVAPAKAKMEGESSKTEMEMEK
CkoeLPAAT1
SEQ ID NO: 84
MAIAAAPVIFLFGLLFFASGLIINLFQAICFVLIWPLSKNAYRRINRVFAELLLSELLCLFDWWAGAKLKLFTDP
ETFRLMGKEHALVITNHKIDLDWMIGWILGQHFGCLGSVISIAKKSTKFLPIFGWSLWFSEYLFLERNWAKDKRT
LKSHIERMKDYPLPLWLILFVEGTRFTRTKLLAAQQYAASSGLPVPRNVLIPHTKGFVSSVSHMRSFVPAIYDVT
VAIPKTSPPPTLIRMFKGQSSVLHVHLKRHLMKDLPESDDAVAQWCRDIFVEKDALLDKHNAEDTFSGQELQETG
RPIKSLLVVISWAVLEVYGAVKFLQWSSLLSSWKGLAFSGIGLGLITLLMHILILFSQSERSTPAKVAPAKPKKE
GESSKTEMEKEK
CkoeLPAAT2
SEQ ID NO: 85
MHVLLEMVTFRFSSFFVFDNVQALCFVLIWPLSKSAYRKINRVFAELLLSELLCLFDWWAGAKLKLFTDPETFRL
MGKEHALVITNHKIDLDWMIGWILGQHFGCLGSVISIAKKSTKFLPIFGWSLWFSEYLFLERNWAKDKRTLKSHI
ERMKDYPLPLWLILFVEGTRFTRTKLLAAQQYAASSGLPVPRNVLIPHTKGFVSSVSHMRSFVPAVYDVTVAFPK
TSPPPTMLSLFEGQSVVLHVHIKRHAMKDLPDSDDAVAQWCRDKFVEKDALLDKHNAEDTFSGQEVHHVGRPIKS
LLVVISWMVVIIFGALKFLQWSSLLSSWKGKAFSAIGLGIATLLMHVLVVFSQADRSNPAKVPPAKLNTELSSSK
KVTNKEN
pSZ2624 PmKASII
SEQ ID NO: 86
gtttaaacGCCGGTCACCACCCGCATGCTCGTACTACAGCGCACGCACCGCTTCGTGA
TCCACCGGGTGAACGTAGTCCTCGACGGAAACATCTGGTTCGGGCCTCCTGCTTG
CACTCCCGCCCATGCCGACAACCTTTCTGCTGTTACCACGACCCACAATGCAACG
CGACACGACCGTGTGGGACTGATCGGTTCACTGCACCTGCATGCAATTGTCACAA
GCGCTTACTCCAATTGTATTCGTTTGTTTTCTGGGAGCAGTTGCTCGACCGCCCGC
GTCCCGCAGGCAGCGATGACGTGTGCGTGGCCTGGGTGTTTCGTCGAAAGGCCA
GCAACCCTAAATCGCAGGCGATCCGGAGATTGGGATCTGATCCGAGTTTGGACC
AGATCCGCCCCGATGCGGCACGGGAACTGCATCGACTCGGCGCGGAACCCAGCT
TTCGTAAATGCCAGATTGGTGTCCGATACCTGGATTTGCCATCAGCGAAACAAGA
CTTCAGCAGCGAGCGTATTTGGCGGGCGTGCTACCAGGGTTGCATACATTGCCCA
TTTCTGTCTGGACCGCTTTACTGGCGCAGAGGGTGAGTTGATGGGGTTGGCAGGC
ATCGAAACGCGCGTGCATGGTGTGCGTGTCTGTTTTCGGCTGCACGAATTCAATA
GTCGGATGGGCGACGGTAGAATTGGGTGTGGCGCTCGCGTGCATGCCTCGCCCC
GTCGGGTGTCATGACCGGGACTGGAATCCCCCCTCGCGACCATCTTGCTAACGCT
CCCGACTCTCCCGACCGCGCGCAGGATAGACTCTTGTTCAACCAATCGACAactagt
ATGcagaccgcccaccagcgcccccccaccgagggccactgcttcggcgcccgcctgcccaccgcctcccgccgcgccgtgc
gccgcgcctggtcccgcatcgcccgcgggcgcgccgccgccgccgccgacgccaaccccgcccgccccgagcgccgcgtggt
gatcaccggccagggcgtggtgacctccctgggccagaccatcgagcagactactcctccctgctggagggcgtgtccggcatct
cccagatccagaagacgacaccaccggctacaccaccaccatcgccggcgagatcaagtccctgcagctggacccctacgtgc
ccaagcgctgggccaagcgcgtggacgacgtgatcaagtacgtgtacatcgccggcaagcaggccctggagtccgccggcctg
cccatcgaggccgccggcctggccggcgccggcctggaccccgccctgtgcggcgtgctgatcggcaccgccatggccggcat
gacctccacgccgccggcgtggaggccctgacccgcggcggcgtgcgcaagatgaaccccactgcatccccactccatctcca
acatgggcggcgccatgctggccatggacatcggatcatgggccccaactactccatctccaccgcctgcgccaccggcaacta
ctgcatcctgggcgccgccgaccacatccgccgcggcgacgccaacgtgatgctggccggcggcgccgacgccgccatcatcc
cctccggcatcggcggcttcatcgcctgcaaggccctgtccaagcgcaacgacgagcccgagcgcgcctcccgcccctgggac
gccgaccgcgacggatcgtgatgggcgagggcgccggcgtgctggtgctggaggagctggagcacgccaagcgccgcggcg
ccaccatcctggccgagctggtgggcggcgccgccacctccgacgcccaccacatgaccgagcccgacccccagggccgcgg
cgtgcgcctgtgcctggagcgcgccctggagcgcgcccgcctggcccccgagcgcgtgggctacgtgaacgcccacggcacct
ccacccccgccggcgacgtggccgagtaccgcgccatccgcgccgtgatcccccaggactccctgcgcatcaactccaccaagt
ccatgatcggccacctgctgggcggcgccggcgccgtggaggccgtggccgccatccaggccctgcgcaccggctggctgcac
cccaacctgaacctggagaaccccgcccccggcgtggaccccgtggtgctggtgggcccccgcaaggagcgcgccgaggacc
tggacgtggtgctgtccaactccttcggcttcggcggccacaactcctgcgtgatcttccgcaagtacgacgagatggactacaag
gaccacgacggcgactacaaggaccacgacatcgactacaaggatcgacgacgacaagTGAatcgatAGATCTCTT
AAGGCAGCAGCAGCTCGGATAGTATCGACACACTCTGGACGCTGGTCGTGTGAT
GGACTGTTGCCGCCACACTTGCTGCCTTGACCTGTGAATATCCCTGCCGCTTTTAT
CAAACAGCCTCAGTGTGTTTGATCTTGTGTGTACGCGCTTTTGCGAGTTGCTAGCT
GCTTGTGCTATTTGCGAATACCACCCCCAGCATCCCCTTCCCTCGTTTCATATCGC
TTGCATCCCAACCGCAACTTATCTACGCTGTCCTGCTATCCCTCAGCGCTGCTCCT
GCTCCTGCTCACTGCCCCTCGCACAGCCTTGGTTTGGGCTCCGCCTGTATTCTCCT
GGTACTGCAACCTGTAAACCAGCACTGCAATGCTGATGCACGGGAAGTAGTGGG
ATGGGAACACAAATGGAAAGCTTAATTAAgagctccgcgtctcgaacagagcgcgcagaggaacgct
gaaggtctcgcctctgtcgcacctcagcgcggcatacaccacaataaccacctgacgaatgcgcttggttcttcgtccattagcgaagc
gtccggttcacacacgtgccacgttggcgaggtggcaggtgacaatgatcggtggagctgatggtcgaaacgttcacagcctaggtg
atatccatcttaaggatctaagtaagattcgaagcgctcgaccgtgccggacggactgcagccccatgtcgtagtgaccgccaatgta
agtgggctggcgtaccctgtacgtgagtcaacgtcactgcacgcgcaccaccctctcgaccggcaggaccaggcatcgcgagatac
agcgcgagccagacacggagtgccgagctatgcgcacgctccaactaggtaccagtttaggtccagcgtccgtggggggggacg
ggctgggagcttgggccgggaagggcaagacgatgcagtccctctggggagtcacagccgactgtgtgtgttgcactgtgcggccc
gcagcactcacacgcaaaatgcctggccgacaggcaggccctgtccagtgcaacatccacggtccctctcatcaggctcaccttgct
cattgacataacggaatgcgtaccgctctttcagatctgtccatccagagaggggagcaggctccccaccgacgctgtcaaacttgctt
cctgcccaaccgaaaacattattgtttgagggggggggggggggggcagattgcatggcgggatatctcgtgaggaacatcactgg
gacactgtggaacacagtgagtgcagtatgcagagcatgtatgctaggggtcagcgcaggaagggggcctttcccagtctcccatgc
cactgcaccgtatccacgactcaccaggaccagcttcttgatcggcttccgctcccgtggacaccagtgtgtagcctctggactccagg
tatgcgtgcaccgcaaaggccagccgatcgtgccgattcctgggtggaggatatgagtcagccaacttggggctcagagtgcacact
ggggcacgatacgaaacaacatctacaccgtgtcctccatgctgacacaccacagcttcgctccacctgaatgtgggcgcatgggcc
cgaatcacagccaatgtcgctgctgccataatgtgatccagaccctctccgcccagatgccgagcggatcgtgggcgctgaatagatt
cctgtttcgatcactgtttgggtcctttccillicgtctcggatgcgcgtctcgaaacaggctgcgtcgggctttcggatcccttttgctccct
ccgtcaccatcctgcgcgcgggcaagttgcttgaccctgggctgataccagggttggagggtattaccgcgtcaggccattcccagcc
cggattcaattcaaagtctgggccaccaccctccgccgctctgtctgatcactccacattcgtgcatacactacgttcaagtcctgatcca
ggcgtgtctcgggacaaggtgtgcttgagtttgaatctcaaggacccactccagcacagctgctggttgaccccgccctcgcaatcta
gaATGgccgcgtccgtccactgcaccctgatgtccgtggtctgcaacaacaagaaccactccgcccgccccaagctgcccaac
tcctccctgctgcccggatcgacgtggtggtccaggccgcggccacccgatcaagaaggagacgacgaccacccgcgccacg
ctgacgacgacccccccacgaccaactccgagcgcgccaagcagcgcaagcacaccatcgacccctcctcccccgacacca
gcccatcccctccacgaggagtgatccccaagtccacgaaggagcacaaggaggtggtgcacgaggagtccggccacgtcct
gaaggtgcccaccgccgcgtgcacctgtccggcggcgagcccgccacgacaactacgacacgtccggcccccagaacgtcaa
cgcccacatcggcctggcgaagctgcgcaaggagtggatcgaccgccgcgagaagctgggcacgccccgctacacgcagatg
tactacgcgaagcagggcatcatcacggaggagatgctgtactgcgcgacgcgcgagaagctggaccccgagacgtccgctc
cgaggtcgcgcggggccgcgccatcatcccctccaacaagaagcacctggagctggagcccatgatcgtgggccgcaagacc
tggtgaaggtgaacgcgaacatcggcaactccgccgtggcctcctccatcgaggaggaggtctacaaggtgcagtgggccacc
atgtggggcgccgacaccatcatggacctgtccacgggccgccacatccacgagacgcgcgagtggatcctgcgcaactccgc
ggtccccgtgggcaccgtccccatctaccaggcgctggagaaggtggacggcatcgcggagaacctgaactgggaggtgacc
gcgagacgctgatcgagcaggccgagcagggcgtggactacttcacgatccacgcgggcgtgctgctgcgctacatccccctga
ccgccaagcgcctgacgggcatcgtgtcccgcggcggctccatccacgcgaagtggtgcctggcctaccacaaggagaacttcg
cctacgagcactgggacgacatcctggacatctgcaaccagtacgacgtcgccctgtccatcggcgacggcctgcgccccggct
ccatctacgacgccaacgacacggcccagacgccgagctgctgacccagggcgagctgacgcgccgcgcgtgggagaagga
cgtgcaggtgatgaacgagggccccggccacgtgcccatgcacaagatccccgagaacatgcagaagcagctggagtggtgc
aacgaggcgcccactacaccctgggccccctgacgaccgacatcgcgcccggctacgaccacatcacctccgccatcggcgcg
gccaacatcggcgccctgggcaccgccctgctgtgctacgtgacgcccaaggagcacctgggcctgcccaaccgcgacgacgt
gaaggcgggcgtcatcgcctacaagatcgccgcccacgcggccgacctggccaagcagcacccccacgcccaggcgtggga
cgacgcgctgtccaaggcgcgatcgagaccgctggatggaccagacgcgctgtccctggaccccatgacggcgatgtccacc
acgacgagacgctgcccgcggacggcgcgaaggtcgcccacactgctccatgtgcggccccaagactgctccatgaagatca
cggaggacatccgcaagtacgccgaggagaacggctacggctccgccgaggaggccatccgccagggcatggacgccatgt
ccgaggagacaacatcgccaagaagacgatctccggcgagcagcacggcgaggtcggcggcgagatctacctgcccgagtc
ctacgtcaaggccgcgcagaagTGAcaattgACGGAGCGTCGTGCGGGAGGGAGTGTGCCGAG
CGGGGAGTCCCGGTCTGTGCGAGGCCCGGCAGCTGACGCTGGCGAGCCGTACGC
CCCGAGGGTCCCCCTCCCCTGCACCCTCTTCCCCTTCCCTCTGACGGCCGCGCCTG
TTCTTGCATGTTCAGCGACggatccTAGGGAGCGACGAGTGTGCGTGCGGGGCTGGC
GGGAGTGGGACGCCCTCCTCGCTCCTCTCTGTTCTGAACGGAACAATCGGCCACC
CCGCGCTACGCGCCACGCATCGAGCAACGAAGAAAACCCCCCGATGATAGGTTG
CGGTGGCTGCCGGGATATAGATCCGGCCGCACATCAAAGGGCCCCTCCGCCAGA
GAAGAAGCTCCTTTCCCAGCAGACTCCTTCTGCTGCCAAAACACTTCTCTGTCCA
CAGCAACACCAAAGGATGAACAGATCAACTTGCGTCTCCGCGTAGCTTCCTCGG
CTAGCGTGCTTGCAACAGGTCCCTGCACTATTATCTTCCTGCTTTCCTCTGAATTA
TGCGGCAGGCGAGCGCTCGCTCTGGCGAGCGCTCCTTCGCGCCGCCCTCGCTGAT
CGAGTGTACAGTCAATGAATGGTCCTGGGCGAAGAACGAGGGAATTTGTGGGTA
AAACAAGCATCGTCTCTCAGGCCCCGGCGCAGTGGCCGTTAAAGTCCAAGACCG
TGACCAGGCAGCGCAGCGCGTCCGTGTGCGGGCCCTGCCTGGCGGCTCGGCGTG
CCAGGCTCGAGAGCAGCTCCCTCAGGTCGCCTTGGACGGCCTCTGCGAGGCCGG
TGAGGGCCTGCAGGAGCGCCTCGAGCGTGGCAGTGGCGGTCGTATCCGGGTCGC
CGGTCACCGCCTGCGACTCGCCATCCgaagagcgtttaaac
pSZ3204 GarmFATA
SEQ ID NO: 87
gctettcGCCGCCGCCACTCCTGCTCGAGCGCGCCCGCGCGTGCGCCGCCAGCGCCTT
GGCCTTTTCGCCGCGCTCGTGCGCGTCGCTGATGTCCATCACCAGGTCCATGAGG
TCTGCCTTGCGCCGGCTGAGCCACTGCTTCGTCCGGGCGGCCAAGAGGAGCATG
AGGGAGGACTCCTGGTCCAGGGTCCTGACGTGGTCGCGGCTCTGGGAGCGGGCC
AGCATCATCTGGCTCTGCCGCACCGAGGCCGCCTCCAACTGGTCCTCCAGCAGCC
GCAGTCGCCGCCGACCCTGGCAGAGGAAGACAGGTGAGGGGGGTATGAATTGTA
CAGAACAACCACGAGCCTTGTCTAGGCAGAATCCCTACCAGTCATGGCTTTACCT
GGATGACGGCCTGCGAACAGCTGTCCAGCGACCCTCGCTGCCGCCGCTTCTCCCG
CACGCTTCTTTCCAGCACCGTGATGGCGCGAGCCAGCGCCGCACGCTGGCGCTGC
GCTTCGCCGATCTGAGGACAGTCGGGGAACTCTGATCAGTCTAAACCCCCTTGCG
CGTTAGTGTTGCCATCCTTTGCAGACCGGTGAGAGCCGACTTGTTGTGCGCCACC
CCCCACACCACCTCCTCCCAGACCAATTCTGTCACCTTTTTGGCGAAGGCATCGG
CCTCGGCCTGCAGAGAGGACAGCAGTGCCCAGCCGCTGGGGGTTGGCGGATGCA
CGCTCAggtaccctttcttgcgctatgacacttccagcaaaaggtagggcgggctgcgagacggcttcccggcgctgcatgcaa
caccgatgatgcttcgaccccccgaagctccttcggggctgcatgggcgctccgatgccgctccagggcgagcgctgtttaaatagc
caggcccccgattgcaaagacattatagcgagctaccaaagccatattcaaacacctagatcactaccacttctacacaggccactcga
gcttgtgatcgcactccgctaagggggcgcctcttcctcttcgtttcagtcacaacccgcaaactctagaatatcaATGctgctgcag
gccttcctgttcctgctggcggcttcgccgccaagatcagcgcctccatgacgaacgagacgtccgaccgccccctggtgcactt
cacccccaacaagggctggatgaacgaccccaacggcctgtggtacgacgagaaggacgccaagtggcacctgtacaccagt
acaacccgaacgacaccgtctgggggacgccatgactggggccacgccacgtccgacgacctgaccaactgggaggaccag
cccatcgccatcgccccgaagcgcaacgactccggcgccactccggctccatggtggtggactacaacaacacctccggcactt
caacgacaccatcgacccgcgccagcgctgcgtggccatctggacctacaacaccccggagtccgaggagcagtacatctcct
acagcctggacggcggctacaccacaccgagtaccagaagaaccccgtgctggccgccaactccacccagaccgcgacccg
aaggtcactggtacgagccctcccagaagtggatcatgaccgcggccaagtcccaggactacaagatcgagatctactcctccg
acgacctgaagtcctggaagctggagtccgcgacgccaacgagggcacctcggctaccagtacgagtgccccggcctgatcga
ggtccccaccgagcaggaccccagcaagtcctactgggtgatgacatctccatcaaccccggcgccccggccggcggctcatc
aaccagtacttcgtcggcagatcaacggcacccacttcgaggccacgacaaccagtcccgcgtggtggacttcggcaaggact
actacgccctgcagaccacttcaacaccgacccgacctacgggagcgccctgggcatcgcgtgggcctccaactgggagtactc
cgccacgtgcccaccaacccctggcgctcctccatgtccctcgtgcgcaagactccctcaacaccgagtaccaggccaacccgg
agacggagctgatcaacctgaaggccgagccgatcctgaacatcagcaacgccggcccctggagccggacgccaccaacac
cacgagacgaaggccaacagctacaacgtcgacctgtccaacagcaccggcaccctggagacgagctggtgtacgccgtcaa
caccacccagacgatctccaagtccgtgacgcggacctctccctctggacaagggcctggaggaccccgaggagtacctccgc
atgggatcgaggtgtccgcgtcctccacacctggaccgcgggaacagcaaggtgaagacgtgaaggagaacccctacttcac
caaccgcatgagcgtgaacaaccagccatcaagagcgagaacgacctgtcctactacaaggtgtacggcagctggaccaga
acatcctggagctgtacttcaacgacggcgacgtcgtgtccaccaacacctacttcatgaccaccgggaacgccctgggctccgt
gaacatgacgacgggggtggacaacctgttctacatcgacaagttccaggtgcgcgaggtcaagTGAcaattgGCAGCA
GCAGCTCGGATAGTATCGACACACTCTGGACGCTGGTCGTGTGATGGACTGTTGC
CGCCACACTTGCTGCCTTGACCTGTGAATATCCCTGCCGCTTTTATCAAACAGCCT
CAGTGTGTTTGATCTTGTGTGTACGCGCTTTTGCGAGTTGCTAGCTGCTTGTGCTA
TTTGCGAATACCACCCCCAGCATCCCCTTCCCTCGTTTCATATCGCTTGCATCCCA
ACCGCAACTTATCTACGCTGTCCTGCTATCCCTCAGCGCTGCTCCTGCTCCTGCTC
ACTGCCCCTCGCACAGCCTTGGTTTGGGCTCCGCCTGTATTCTCCTGGTACTGCAA
CCTGTAAACCAGCACTGCAATGCTGATGCACGGGAAGTAGTGGGATGGGAACAC
AAATGGAggatcccgcgtctcgaacagagcgcgcagaggaacgctgaaggtctcgcctctgtcgcacctcagcgcggcata
caccacaataaccacctgacgaatgcgcttggttcttcgtccattagcgaagcgtccggttcacacacgtgccacgttggcgaggtgg
caggtgacaatgatcggtggagctgatggtcgaaacgttcacagcctagggatatcctgaagaatgggaggcaggtgttgttgattat
gagtgtgtaaaagaaaggggtagagagccgtcctcagatccgactactatgcaggtagccgctcgcccatgcccgcctggctgaata
ttgatgcatgcccatcaaggcaggcaggcatactgtgcacgcaccaagcccacaatcttccacaacacacagcatgtaccaacgcac
gcgtaaaagttggggtgctgccagtgcgtcatgccaggcatgatgtgctcctgcacatccgccatgatctcctccatcgtctcgggtgtt
tccggcgcctggtccgggagccgttccgccagatacccagacgccacctccgacctcacggggtactittcgagcgtctgccggtag
tcgacgatcgcgtccaccatggagtagccgaggcgccggaactggcgtgacggagggaggagagggaggagagagagggggg
ggggggggggggatgattacacgccagtctcacaacgcatgcaagacccgtttgattatgagtacaatcatgcactactagatggatg
agcgccaggcataaggcacaccgacgttgatggcatgagcaactcccgcatcatatttcctattgtcctcacgccaagccggtcaccat
ccgcatgctcatattacagcgcacgcaccgcttcgtgatccaccgggtgaacgtagtcctcgacggaaacatctggctcgggcctcgt
gctggcactccctcccatgccgacaacctttctgctgtcaccacgacccacgatgcaacgcgacacgacccggtgggactgatcggtt
cactgcacctgcatgcaattgtcacaagcgcatactccaatcgtatccgtttgatttctgtgaaaactcgctcgaccgcccgcgtcccgc
aggcagcgatgacgtgtgcgtgacctgggtgtttcgtcgaaaggccagcaaccccaaatcgcaggcgatccggagattgggatctg
atccgagcttggaccagatcccccagatgcggcacgggaactgcatcgactcggcgcggaacccagctttcgtaatgccagattg
gtgtccgataccttgatttgccatcagcgaaacaagacttcagcagcgagcgtatttggcgggcgtgctaccagggttgcatacattgc
ccatttctgtctggaccgctttaccggcgcagagggtgagttgatggggttggcaggcatcgaaacgcgcgtgcatggtgtglgtgtct
gttttcggctgcacaatttcaatagtcggatgggcgacggtagaattgggtgttgcgctcgcgtgcatgcctcgccccgtcgggtgtcat
gaccgggactggaatcccccctcgcgaccctcctgctaacgctcccgactctcccgcccgcgcgcaggatagactctagttcaacca
atcgacaactagtATGgccaccgcatccactactcggcgacaatgcccgctgcggcgacctgcgtcgctcggcgggctccggg
ccccggcgcccagcgaggcccctccccgtgcgcgggcgcgccatccccccccgcatcatcgtggtgtcctcctcctcctccaagg
tgaaccccctgaagaccgaggccgtggtgtcctccggcctggccgaccgcctgcgcctgggctccctgaccgaggacggcctgt
cctacaaggagaagacatcgtgcgctgctacgaggtgggcatcaacaagaccgccaccgtggagaccatcgccaacctgctgc
aggaggtgggctgcaaccacgcccagtccgtgggctactccaccggcggcactccaccacccccaccatgcgcaagctgcgcc
tgatctgggtgaccgcccgcatgcacatcgagatctacaagtaccccgcctggtccgacgtggtggagatcgagtcctggggcca
gggcgagggcaagatcggcacccgccgcgactggatcctgcgcgactacgccaccggccaggtgatcggccgcgccacctcc
aagtgggtgatgatgaaccaggacacccgccgcctgcagaaggtggacgtggacgtgcgcgacgagtacctggtgcactgccc
ccgcgagctgcgcctggccaccccgaggagaacaactcctccctgaagaagatctccaagctggaggacccctcccagtactc
caagctgggcctggtgccccgccgcgccgacctggacatgaaccagcacgtgaacaacgtgacctacatcggctgggtgctgg
agtccatgccccaggagatcatcgacacccacgagctgcagaccatcaccctggactaccgccgcgagtgccagcacgacga
cgtggtggactccctgacctcccccgagccctccgaggacgccgaggccgtgacaaccacaacggcaccaacggctccgcca
acgtgtccgccaacgaccacggctgccgcaacacctgcacctgctgcgcctgtccggcaacggcctggagatcaaccgcggcc
gcaccgagtggcgcaagaagcccacccgcATGGACTACAAGGACCACGACGGCGACTACAAGGA
CCACGACATCGACTACAAGGACGACGACGACAAGTGAategatagatctcttaagGCAGCAG
CAGCTCGGATAGTATCGACACACTCTGGACGCTGGTCGTGTGATGGACTGTTGCC
GCCACACTTGCTGCCTTGACCTGTGAATATCCCTGCCGCTTTTATCAAACAGCCTC
AGTGTGTTTGATCTTGTGTGTACGCGCTTTTGCGAGTTGCTAGCTGCTTGTGCTAT
TTGCGAATACCACCCCCAGCATCCCCTTCCCTCGTTTCATATCGCTTGCATCCCAA
CCGCAACTTATCTACGCTGTCCTGCTATCCCTCAGCGCTGCTCCTGCTCCTGCTCA
CTGCCCCTCGCACAGCCTTGGTTTGGGCTCCGCCTGTATTCTCCTGGTACTGCAAC
CTGTAAACCAGCACTGCAATGCTGATGCACGGGAAGTAGTGGGATGGGAACACA
AATGGAaagcttaattaagagctcTTGTTTTCCAGAAGGAGTTGCTCCTTGAGCCTTTCATTC
TCAGCCTCGATAACCTCCAAAGCCGCTCTAATTGTGGAGGGGGTTCGAATTTAAA
AGCTTGGAATGTTGGTTCGTGCGTCTGGAACAAGCCCAGACTTGTTGCTCACTGG
GAAAAGGACCATCAGCTCCAAAAAACTTGCCGCTCAAACCGCGTACCTCTGCTTT
CGCGCAATCTGCCCTGTTGAAATCGCCACCACATTCATATTGTGACGCTTGAGCA
GTCTGTAATTGCCTCAGAATGTGGAATCATCTGCCCCCTGTGCGAGCCCATGCCA
GGCATGTCGCGGGCGAGGACACCCGCCACTCGTACAGCAGACCATTATGCTACC
TCACAATAGTTCATAACAGTGACCATATTTCTCGAAGCTCCCCAACGAGCACCTC
CATGCTCTGAGTGGCCACCCCCCGGCCCTGGTGCTTGCGGAGGGCAGGTCAACC
GGCATGGGGCTACCGAAATCCCCGACCGGATCCCACCACCCCCGCGATGGGAAG
AATCTCTCCCCGGGATGTGGGCCCACCACCAGCACAACCTGCTGGCCCAGGCGA
GCGTCAAACCATACCACACAAATATCCTTGGCATCGGCCCTGAATTCCTTCTGCC
GCTCTGCTACCCGGTGCTTCTGTCCGAAGCAGGGGTTGCTAGGGATCGCTCCGAG
TCCGCAAACCCTTGTCGCGTGGCGGGGCTTGTTCGAGCTTgaagagc
pSZ4198 (BnLPAT2)
SEQ ID NO: 88
gctcttccgctAACGGAGGTCTGTCACCAAATGGACCCCGTCTATTGCGGGAAACCACG
GCGATGGCACGTTTCAAAACTTGATGAAATACAATATTCAGTATGTCGCGGGCGG
CGACGGCGGGGAGCTGATGTCGCGCTGGGTATTGCTTAATCGCCAGCTTCGCCCC
CGTCTTGGCGCGAGGCGTGAACAAGCCGACCGATGTGCACGAGCAAATCCTGAC
ACTAGAAGGGCTGACTCGCCCGGCACGGCTGAATTACACAGGCTTGCAAAAATA
CCAGAATTTGCACGCACCGTATTCGCGGTATTTTGTTGGACAGTGAATAGCGATG
CGGCAATGGCTTGTGGCGTTAGAAGGTGCGACGAAGGTGGTGCCACCACTGTGC
CAGCCAGTCCTGGCGGCTCCCAGGGCCCCGATCAAGAGCCAGGACATCCAAACT
ACCCACAGCATCAACGCCCCGGCCTATACTCGAACCCCACTTGCACTCTGCAATG
GTATGGGAACCACGGGGCAGTCTTGTGTGGGTCGCGCCTATCGCGGTCGGCGAA
GACCGGGAAggtaccgcggtgagaatcgaaaatgcatcgtttctaggttcggagacggtcaattccctgctccggcgaatctg
tcggtcaagctggccagtggacaatgttgctatggcagcccgcgcacatgggcctcccgacgcggccatcaggagcccaaacagc
gtgtcagggtatgtgaaactcaagaggtccctgctgggcactccggccccactccgggggcgggacgccaggcattcgcggtcggt
cccgcgcgacgagcgaaatgatgattcggttacgagaccaggacgtcgtcgaggtcgagaggcagcctcggacacgtctcgctag
ggcaacgccccgagtccccgcgagggccgtaaacattgtttctgggtgtcggagtgggcattttgggcccgatccaatcgcctcatgc
cgctctcgtctggtcctcacgttcgcgtacggcctggatcccggaaagggcggatgcacgtggtgttgccccgccattggcgcccac
gtttcaaagtccccggccagaaatgcacaggaccggcccggctcgcacaggccatgctgaacgcccagatttcgacagcaacacca
tctagaataatcgcaaccatccgcgttttgaacgaaacgaaacggcgctgtttagcatgtttccgacatcgtgggggccgaagcatgct
ccggggggaggaaagcgtggcacagcggtagcccattctgtgccacacgccgacgaggaccaatccccggcatcagccttcatcg
acggctgcgccgcacatataaagccggacgcctaaccggtttcgtggttatgactagtATGttcgcgttctacttcctgacggcctgc
atctccctgaagggcgtgacggcgtctccccctcctacaacggcctgggcctgacgccccagatgggctgggacaactggaaca
cgacgcctgcgacgtctccgagcagctgctgctggacacggccgaccgcatctccgacctgggcctgaaggacatgggctaca
agtacatcatcctggacgactgctggtcctccggccgcgactccgacggcacctggtcgccgacgagcagaagaccccaacgg
catgggccacgtcgccgaccacctgcacaacaactccacctgacggcatgtactcctccgcgggcgagtacacgtgcgccggct
accccggctccctgggccgcgaggaggaggacgcccagacttcgcgaacaaccgcgtggactacctgaagtacgacaactgc
tacaacaagggccagacggcacgcccgagatctcctaccaccgctacaaggccatgtccgacgccctgaacaagacgggccg
ccccatcactactccctgtgcaactggggccaggacctgaccactactggggctccggcatcgcgaactcctggcgcatgtccgg
cgacgtcacggcggagacacgcgccccgactcccgctgcccctgcgacggcgacgagtacgactgcaagtacgccggcacc
actgctccatcatgaacatcctgaacaaggccgcccccatgggccagaacgcgggcgtcggcggctggaacgacctggacaa
cctggaggtcggcgtcggcaacctgacggacgacgaggagaaggcgcacactccatgtgggccatggtgaagtcccccctgat
catcggcgcgaacgtgaacaacctgaaggcctcctcctactccatctactcccaggcgtccgtcatcgccatcaaccaggactcc
aacggcatccccgccacgcgcgtctggcgctactacgtgtccgacacggacgagtacggccagggcgagatccagatgtggtc
cggccccctggacaacggcgaccaggtcgtggcgctgctgaacggcggctccgtgtcccgccccatgaacacgaccctggagg
agatcacttcgactccaacctgggctccaagaagctgacctccacctgggacatctacgacctgtgggcgaaccgcgtcgacaa
ctccacggcgtccgccatcctgggccgcaacaagaccgccaccggcatcctgtacaacgccaccgagcagtcctacaaggacg
gcctgtccaagaacgacacccgcctgacggccagaagatcggctccctgtcccccaacgcgatcctgaacacgaccgtccccg
cccacggcatcgcgactaccgcctgcgcccctcctccTGAtacgtactcgagGCAGCAGCAGCTCGGATAGT
ATCGACACACTCTGGACGCTGGTCGTGTGATGGACTGTTGCCGCCACACTTGCTG
CCTTGACCTGTGAATATCCCTGCCGCTTTTATCAAACAGCCTCAGTGTGTTTGATC
TTGTGTGTACGCGCTTTTGCGAGTTGCTAGCTGCTTGTGCTATTTGCGAATACCAC
CCCCAGCATCCCCTTCCCTCGTTTCATATCGCTTGCATCCCAACCGCAACTTATCT
ACGCTGTCCTGCTATCCCTCAGCGCTGCTCCTGCTCCTGCTCACTGCCCCTCGCAC
AGCCTTGGTTTGGGCTCCGCCTGTATTCTCCTGGTACTGCAACCTGTAAACCAGC
ACTGCAATGCTGATGCACGGGAAGTAGTGGGATGGGAACACAAATGGAAagctgtag
aattcctggctcgggcctcgtgctggcactccctcccatgccgacaacctttctgctgtcaccacgacccacgatgcaacgcgacacg
acccggtgggactgatcggttcactgcacctgcatgcaattgtcacaagcgcatactccaatcgtatccgtttgatttctgtgaaaactcg
ctcgaccgcccgcgtcccgcaggcagcgatgacgtgtgcgtgacctgggtgtttcgtcgaaaggccagcaaccccaaatcgcaggc
gatccggagattgggatctgatccgagcttggaccagatcccccacgatgcggcacgggaactgcatcgactcggcgcggaaccca
gctttcgtaaatgccagattggtgtccgataccttgatttgccatcagcgaaacaagacttcagcagcgagcgtatttggcgggcgtgct
accagggttgcatacattgcccatttctgtctggaccgctttaccggcgcagagggtgagttgatggggttggcaggcatcgaaacgc
gcgtgcatggtgtgtgtgtctgttttcggctgcacaatttcaatagtcggatgggcgacggtagaattgggtgttgcgctcgcgtgcatgc
ctcgccccgtcgggtgtcatgaccgggactggaatcccccctcgcgaccctcctgctaacgctcccgactctcccgcccgcgcgcag
gatagactctagttcaaccaatcgacaactagtATGgccatggccgccgccgtgatcgtgcccctgggcatcctgttcttcatctcc
ggcctggtggtgaacctgctgcaggccatctgctacgtgctgatccgccccctgtccaagaacacctaccgcaagatcaaccgcg
tggtggccgagaccctgtggctggagctggtgtggatcgtggactggtgggccggcgtgaagatccaggtgacgccgacaacg
agaccttcaaccgcatgggcaaggagcacgccctggtggtgtgcaaccaccgctccgacatcgactggctggtgggctggatcc
tggcccagcgctccggctgcctgggctccgccctggccgtgatgaagaagtcctccaagacctgcccgtgatcggctggtccatgt
ggactccgagtacctgacctggagcgcaactgggccaaggacgagtccaccctgaagtccggcctgcagcgcctgaacgactt
cccccgccccactggctggccctgacgtggagggcacccgatcaccgaggccaagctgaaggccgcccaggagtacgccgc
ctcctccgagctgcccgtgccccgcaacgtgctgatcccccgcaccaagggatcgtgtccgccgtgtccaacatgcgctccacgt
gcccgccatctacgacatgaccgtggccatccccaagacctcccccccccccaccatgctgcgcctgacaagggccagccctcc
gtggtgcacgtgcacatcaagtgccactccatgaaggacctgcccgagtccgacgacgccatcgcccagtggtgccgcgacca
gacgtggccaaggacgccctgctggacaagcacatcgccgccgacaccaccccggccagcaggagcagaacatcggccgc
cccatcaagtccctggccgtggtgctgtcctggtcctgcctgctgatcctgggcgccatgaagacctgcactggtccaacctgactc
ctcctggaagggcatcgccactccgccctgggcctgggcatcatcaccctgtgcatgcagatcctgatccgctcctcccagtccga
gcgctccacccccgccaaggtggtgcccgccaagcccaaggacaaccacaacgactccggctcctcctcccagaccgaggtg
gagaagcagaagTGAatcgatagatctcttaagGCAGCAGCAGCTCGGATAGTATCGACACACT
CTGGACGCTGGTCGTGTGATGGACTGTTGCCGCCACACTTGCTGCCTTGACCTGT
GAATATCCCTGCCGCTTTTATCAAACAGCCTCAGTGTGTTTGATCTTGTGTGTACG
CGCTTTTGCGAGTTGCTAGCTGCTTGTGCTATTTGCGAATACCACCCCCAGCATCC
CCTTCCCTCGTTTCATATCGCTTGCATCCCAACCGCAACTTATCTACGCTGTCCTG
CTATCCCTCAGCGCTGCTCCTGCTCCTGCTCACTGCCCCTCGCACAGCCTTGGTTT
GGGCTCCGCCTGTATTCTCCTGGTACTGCAACCTGTAAACCAGCACTGCAATGCT
GATGCACGGGAAGTAGTGGGATGGGAACACAAATGGAaagettaattaagagatcAGCGG
CGACGGTCCTGCTACCGTACGACGTTGGGCACGCCCATGAAAGTTTGTATACCGA
GCTTGTTGAGCGAACTGCAAGCGCGGCTCAAGGATACTTGAACTCCTGGATTGAT
ATCGGTCCAATAATGGATGGAAAATCCGAACCTCGTGCAAGAACTGAGCAAACC
TCGTTACATGGATGCACAGTCGCCAGTCCAATGAACATTGAAGTGAGCGAACTGT
TCGCTTCGGTGGCAGTACTACTCAAAGAATGAGCTGCTGTTAAAAATGCACTCTC
GTTCTCTCAAGTGAGTGGCAGATGAGTGCTCACGCCTTGCACTTCGCTGCCCGTG
TCATGCCCTGCGCCCCAAAATTTGAAAAAAGGGATGAGATTATTGGGCAATGGA
CGACGTCGTCGCTCCGGGAGTCAGGACCGGCGGAAAATAAGAGGCAACACACTC
CGCTTCTTAgctcttc
pSZ4198 BnLPAT2(1.5)
SEQ ID NO: 89
ATGgccatggccgccgccgccgtgatcgtgcccctgggcatcctgacttcatctccggcctggtggtgaacctgctgcaggccgt
gtgctacgtgctgatccgccccctgtccaagaacacctaccgcaagatcaaccgcgtggtggccgagaccctgtggctggagctg
gtgtggatcgtggactggtgggccggcgtgaagatccaggtgacgccgacgacgagaccacaaccgcatgggcaaggagca
cgccctggtggtgtgcaaccaccgctccgacatcgactggctggtgggctggatcctggcccagcgctccggctgcctgggctcc
gccctggccgtgatgaagaagtcctccaagacctgcccgtgatcggctggtccatgtggactccgagtacctgacctggagcgca
actgggccaaggacgagtccaccctgaagtccggcctgcagcgcctgaacgacacccccgccccactggctggccctgacgtg
gagggcacccgatcaccgaggccaagctgaaggccgcccaggagtacgccgcctcctcccagctgcccgtgccccgcaacgt
gctgatcccccgcaccaagggcttcgtgtccgccgtgtccaacatgcgctccttcgtgcccgccatctacgacatgaccgtggccat
ccccaagacctcccccccccccaccatgctgcgcctgacaagggccagccctccgtggtgcacgtgcacatcaagtgccactcc
atgaaggacctgcccgagtccgacgacgccatcgcccagtggtgccgcgaccagacgtggccaaggacgccctgctggacaa
gcacatcgccgccgacaccaccccggccagaaggagcacaacatcggccgccccatcaagtccctggccgtggtggtgtcctg
ggcctgcctgctgaccctgggcgccatgaagacctgcactggtccaacctgactcctccctgaagggcatcgccctgtccgccctg
ggcctgggcatcatcaccctgtgcatgcagatcctgatccgctcctcccagtccgagcgctccacccccgccaaggtggcccccg
ccaagcccaaggacaagcaccagtccggctcctcctcccagaccgaggtggaggagaagcagaagTGA
pSZ4206 TcLPAT2 GhomLPAT2A
SEQ ID NO: 90
ATGgccatcgccgccgccgccgtgatcgtgcccctgggcctgctgttcttcatctccggcctggtggtgaacctgatccaggccctgtgcttc
gtgctgatccgccccctgtccaagaacacctaccgcaagatcaaccgcgtggtggccgagctgctgtggctggagctgatctggctggtgg
actggtgggccggcgtgaagatcaaggtgttcatggaccccgagtccttcaacctgatgggcaaggagcacgccctggtggtggccaacc
accgctccgacatcgactggctggtgggctggctgctggcccagcgctccggctgcctgggctccgccctggccgtgatgaagaagtcctcc
aagttcctgcccgtgatcggctggtccatgtggttctccgagtacctgttcctggagcgctcctgggccaaggacgagaacaccctgaaggc
cggcctgcagcgcctgaaggacttcccccgccccttctggctggccttcttcgtggagggcacccgcttcacccaggccaagttcctggccgc
ccaggagtacgccgcctcccagggcctgcccatcccccgcaacgtgctgatcccccgcaccaagggcttcgtgtccgccgtgtcccacatgc
gctccttcgtgcccgccatctacgacatgaccgtggccatccccaagtcctccccctcccccaccatgctgcgcctgttcaagggccagccctc
cgtggtgcacgtgcacatcaagcgctgcctgatgaaggagctgcccgagaccgacgaggccgtggcccagtggtgcaaggacatgttcg
tggagaaggacaagctgctggacaagcacatcgccgaggacaccttctccgaccagcccatgcaggacctgggccgccccatcaagtcc
ctgctggtggtggcctcctgggcctgcctgatggcctacggcgccctgaagttcctgcagtgctcctccctgctgtcctcctggaagggcatcg
ccttcttcctggtgggcctggccatcgtgaccatcctgatgcacatcctgatcctgttctcccagtccgagcgctccacccccgccaaggtggc
ccccggcaagcccaagaacgacggcgagacctccgaggcccgccgcgacaagcagcagTGA
Nucleotide sequence of the GhomLPAT2A coding sequence, used in the
transforming DNA from pSZ4412.
SEQ ID NO: 91
ATGgccatccccgccgccatcgtgatcgtgcccgtgggcctgctgttcttcatctccggcctgatcgtgaacctgctgcaggccctgtgcttcg
tgctgatccgccccctgtccaagtccgcctaccgcaccatcaaccgccagctggtggagctgctgtggctggagctggtgtgcatcgtggac
tggtgggcccgcgtgaagatccagctgttcaccgacaaggagaccctgaactccatgggcaaggagcacgccctggtgatgtgcaacca
ccgctccgacatcgactggctggtgggctggatcctggcccagcgctccggctgcctgggctccaccgtggccgtgatgaagaagtcctcca
aggtgctgcccgtgatcggctggtccatgtggttctccgagtacctgttcctggagcgcaactgggccaaggacgagtccaccctgaagtcc
ggcctgcagcgcctgcgcgacttcccccgccccttctggctggccctgttcgtggagggcacccgcttcacccagcccaagctgctggccgcc
caggagtacgccgcctccaccggcctgcccatcccccgcaacgtgctgatcccccgcaccaagggcttcgtgtccgccgtgtccatcacccgc
tccttcgtgcccgtgatctacgacatcaccgtggccatccccaagtcctccccccagcccaccatgctgcgcctgttcaagggccagtcctccg
tggtgcacgtgcacctgaagcgccacctgatgaaggacctgcccgagtccgacgacgacgtggcccagtggtgccgcgaccagttcgtgg
tgaaggactccctgctggacaagcacatcgccgaggacaccttctccgaccaggagctgcaggacatcggccgccccatcaagtccctgg
tggtgttcacctcctgggtgtgcatcatcaccttcggcgccctgaagttcctgcagtggtcctccctgctgcactcctggaagggcatcgccat
ctccgcctccggcctggccatcgtgaccgtgctgatgcacatcctgatccgcttctcccagtccgagcactccacctccgccaagatcgccgcc
gagaagcacaagaacggcggcgtgtcccaggagatgggccgcgagaagcagcacTGA
Nucleotide sequence of the GhomLPAT2B coding sequence, used in the
transforming DNA from pSZ4413.
SEQ ID NO: 92
ATGgagatccccgccgtggccgtgatcgtgcccatcggcatcctgttcttcatctccggcctgatcgtgaacctgatgcaggccatctgcttc
ttcctgatccgccccctgtccaagaacacccaccgcatcgtgaaccgccagctggccgagctgctgtggctggagctgatctggatcgtgga
ctggtgggccggcgtgaagatccagctgttcaccgacaaggagaccctgcacctgatgggcaaggagcacgccctggtgatctgcaacc
actcctccgacatcgactggctggtgggctggctgctgtgccagcgctccggctgcctgggctccgccctggccgtgatgaagtcctcctcca
aggtgctgcccgtgatcggctggtccatgtggttctccgagtacctgttcctggagcgctcctgggccaaggacgagtccaccctgaagtcc
ggcctgcagcgcctgaaggacttcccccgccccttctggctggccctgttcgtggagggcacccgcttcacccaggccaagctgctggccgc
ccaggagtacgccatgtccgccggcctgcccgtgccccgcaacgtgctgatcccccgcaccaagggcttcgtgtccgccgtgtccaacatgc
gctccttcgtgcccgccatctacgacgtgaccgtggccatccccaagtcctccgtgcagcccaccatgctgcgcctgttcaagggccagtcctc
cgtggtgcaggtgcacctgaagcgccactccatgaaggacctgcccgagtccgaggacgacgtggcccagtggtgccgcgaccgcttcgt
ggtgaaggactccctgctggacaagcacaaggtggaggacaccttcaccgaccaggagctgcaggacctgggccgccccatcaagtccc
tggtggtggtgacctgctgggcctgcatcatcatcttcggcatcctgaagttcctgcagtggtcctccctgctgtactcctggaagggcatggc
catctccgcctccggcctggccgtggtgaccttcctgatgcagatcctgatccgcttctcccagtccgagcgctccacccccgccaagatcgcc
cccgccaagcccaacaaggccggcaactcctccgagaccgtgcgcgacaagcaccagTGA
Nucleotide sequence of the GhomLPAt2C coding sequence, used in the
transforming DNA from pSZ4414.
SEQ ID NO: 93
ATGgccatccccgccgccatcatcatcgtgcccctgggcctgatcttcttcacctccggcctgatcatcaacctgatccaggccgtgtgctacg
tgctgatccgccccctgtccaagtccaccttccgccgcatcaaccgcgagctggccgagctgctgtggctggagctggtgtgggtggtggac
tggtgggccggcgtgaagatccagctgttcaccgacaaggagaccctgcactccatgggcaaggagcacgccctggtgatctgcaaccac
cgctccgacatcgactggctggtgggctggatcctggcccagcgctccggctgcctgggctccgccctggccgtgatgaagaagtcctccaa
ggtgctgcccgtgatcggctggtccatgtggttctccgagtacttcttcctggagcgcaactgggccatggacgagtccaccctgaagtccg
gcctgcagcgcctgaaggacttcccccagcccttctggctggccctgttcgtggagggcacccgcttcacccagcccaagctgctggccgccc
aggagtacgccgcctccgccggcctgcccatcccccgcaacgtgctgatcccccgcaccaagggcttcgtgtccgccgtgaacatcatgcgc
tccttcgtgcccgccatctacgacgtgaccgtggccatccccaagtcctccccccagcccaccatgctgcgcctgttcaagggccagtcctccg
tggtgcacgtgcacctgaagcgccacctgatggaggacctgcccgagaccgacgacgacgtggcccagtggtgccgcgaccgcttcgtgg
tgaaggactccctgctggacaagtacgtggccgaggacaccttctccgaccaggagctgcaggacctgggccgccccatcaagtccctgg
tggtggtgacctcctgggtgtgcatcatcgccttcggctccctgaagttcctgcagtggtcctccctgctgtactcctggaagggcatcgtgat
ctccgccgcctccctggccgtggtgaccgtgctgatgcagatcctgatccgcttctcccagtccgagcgctccacctccgccaagatcgccgcc
gccaagcgcaagaacgtgggcgagcacTGA
Nucleotide sequence of the GindPAT2A coding sequence, used in the transforming
DNA from pSZ4415.
SEQ ID NO: 94
ATGgccatccccgtggtggtggtgatcgtgcccgtgggcctgctgttcttcatctccggcctgatcgtgaacctgctgcaggccctgtgcttc
gtgctgatccgccccctgtccaagtccgcctaccgcaccatcaaccgccagctggtggagctgctgtggctggagctggtgtgcatcgtgga
ctggtgggcccgcgtgaagatccagctgttcatcgacaaggagaccctgaactccatgggcaaggagcacgccctggtgatgtgcaacc
accgctcctacatcgactggctggtgggctggatcctggcccagcgctccggctgcctgggctccaccgtggccgtgatgaagaagtcctcc
aaggtgctgcccgtgatcggctggtccatgtggttctccgagtacctgttcctggagcgcaactgggccaaggacgagtccaccctgaagt
ccggcctgcagcgcctgcgcgacttcccccgccccttctggctggccctgttcgtggagggcacccgcttcacccagcccaagctgctggccg
cccaggagtacgccgcctccaccggcctgcccatcccccgcaacgtgctgatcccccgcaccaagggcttcgtgtccgccgtgtccatcaccc
gctccttcgtgcccgtgatctacgacatcaccgtggccatccccaagtcctcctcccagcccaccatgctgaagctgttcaagggccagtcctc
cgtggtgcacgtgcacctgaagcgccacctgatgaaggacctgcccgagtccgacgacgacgtggcccagtggtgccgcgcccagttcgt
ggtgaaggactccctgctggacaagcacatcgccgaggacaccttctccgaccaggagctgcaggacatcggccgccccatcaagtccct
ggtggtgttcacctcctgggtgtgcatcatcaccttcggcgccctgaagttcctgcagtggtcctccctgctgcactcctggaagggcatcgcc
atctccgcctccggcctggccatcgtgaccgtgctgatgcacatcctgatccgcttctcccagtccgagcactccacctccgccaagatcgccg
ccgagaagcacaagaacggcggcgtgtcccaggagatgggccgcgagaagcagcacTGA
Nucleotide sequence of the GindPAT2B coding sequence, used in the transofrming
DNA from pSZ4416.
SEQ ID NO: 95
ATGggcatccccgccgtggccgtgatcgtgcccatcggcatcctgttcttcatctccggcttcatcgtgaacctgatgcaggccatctgcttcg
tgctgatccgccccctgtccaagaacacctaccgcatcgtgaaccgccagctggccgagttcctgtggctggagctgatctgggtggtggac
tggtgggccggcgtgaagatccagctgttcaccgacaaggagaccctgcacctgatgggcaaggagcacgccctggtgatctgcaacca
ccgctccgacatcgactggctggtgggctggctgctgtgccagcgctccggctgcctgggctccgccctggccgtgatgaagtcctcctccaa
ggtgctgcccgtgatcggctggtccatgtggttctccgagtacctgttcctggagcgctcctgggccaaggacgagtccaccctgaagctgg
gcctgcagcgcctgaaggacttcccccgccccttctggctggccctgttcgtggagggcacccgcttcacccaggccaagctgctggccgccc
aggagtacgccatgtccgccggcctgcccgtgccccgcaacgtgctgatcccccgcaccaagggcttcgtgtccgccgtgtccaacatgcgc
tccttcgtgcccgccatctacgacgtgaccgtggccatccccaagtcctccgtgcagcccaccatgctgggcctgttcaagggccagtcctgc
gtggtgcaggtgcacctgaagcgccacctgatgaaggacctgcccgagtccgaggacgacgtggcccagtggtgccgcgagcgcttcgt
ggtgaaggactccctgctggacaagcacaaggtggaggacaccttctccgaccaggagctgcaggacctgggccgccccatcaagtccct
ggtggtggtgatctcctgggcctgcatcctgatcttctggatcctgaagttcctgcagtggtcctccctgctgtactcctggaagggcatcgcc
atctccgcctgcgccatggccgtgatcgccttcctgatgcagatcctgctgcgcttctcccagtccgagcgctccacccccgccaagatcgccc
ccgccaagcccaacaacgcccgcaactcctccgagaccgtgcgcgacaagcaccagTGA
Nucleotide sequence of the GindPAT2C coding sequence, used in the transforming
DNA from pSZ4417.
SEQ ID NO: 96
ATGgccatccccgccgccatcatcatcgtgcccctgggcctgatcttcttcacctccggcttcatcatcaacctgatccaggccgtgtgctacg
tgctgatccgccccctgtccaagtccaccttccgccgcatcaaccgccagctggccgagctgctgtggctggagctggtgtgggtggtggac
tggtgggccggcgtgaagatccagctgttcaccaacaaggagaccctgcactccatcggcaaggagcacgccctggtgatctgcaaccag
cgctccgacatcgactggctggtgggctggatcctggcccagcgctccggctgcctgggctccgccctggccgtgatgaagaagtcctccaa
ggtgctgcccgtgatcggctggtccatgtggttctccgagtacctgttcctggagcgcaactgggccatggacgagtccaccctgaagtccg
gcctgcagtggctgaaggacttcccccagcccttctggctggccctgttcgtggagggcacccgcttcacccagcccaagctgctggccgcc
caggagtacgccgcctccgccggcctgcccatcccccgcaacgtgctgatcccccgcaccaagggcttcgtgtccgccgtgaacatcatgcg
ctccttcgtgcccgccgtgtacgacgtgaccgtggccatccccaagtcctccccccagcccaccatgctgcgcctgttcaagggccagtcctcc
gtggtgcacgtgcacctgaagcgccacctgatggaggacctgcccgagaccgacgacgacgtggcccagtggtgccgcgaccgcttcgtg
gtgaaggactccctgctggacaagcacctggccgaggacaccttctccgaccaggagctgcaggacctgggccgccccatcaagtccctg
gtggtggtgacctcctgggtgtgcatcatcgccttcggcgccctgaagttcctgcagtggtcctccctgctgtactcctggaagggcatcgtg
atctccgccgcctccctggccgtggtgaccgtgctgatgcagatcctgatccgcttctcccagtccgagcgctccacctccgccaaggtggtg
gccgagaagcgcaagaacgtgggcgagcacTGA
pSZEX61 Transorming DNA expressing CnLPAAT.
SEQ ID NO: 97
gtttaaacgccggtcaccacccgcatgctcgtactacagcgcacgcaccgcttcgtgatccaccgggtgaacgtagtcctcgacgg
aaacatctggttcgggcctcctgcttgcactcccgcccatgccgacaacctttctgctgttaccacgacccacaatgcaacgcgaca
cgaccgtgtgggactgatcggttcactgcacctgcatgcaattgtcacaagcgcttactccaattgtattcgtttgttttctgggagc
agttgctcgaccgcccgcgtcccgcaggcagcgatgacgtgtgcgtggcctgggtgtttcgtcgaaaggccagcaaccctaaatcg
caggcgatccggagattgggatctgatccgagtttggaccagatccgccccgatgcggcacgggaactgcatcgactcggcgcgg
aacccagctttcgtaaatgccagattggtgtccgatacctggatttgccatcagcgaaacaagacttcagcagcgagcgtatttgg
cgggcgtgctaccagggttgcatacattgcccatttctgtctggaccgctttactggcgcagagggtgagttgatggggttggcagg
catcgaaacgcgcgtgcatggtgtgcgtgtctgttttcggctgcacgaattcaatagtcggatgggcgacggtagaattgggtgtg
gcgctcgcgtgcatgcctcgccccgtcgggtgtcatgaccgggactggaatcccccctcgcgaccatcttgctaacgctcccgactc
tcccgaccgcgcgcaggatagactcttgttcaaccaatcgacaggtaccATGgacgcctccggcgcctcctccttcctgcgcggccgct
gcctggagtcctgcttcaaggcctccttcggctacgtaatgtcccagcccaaggacgccgccggccagccctcccgccgccccgccgacgcc
gacgacttcgtggacgacgaccgctggatcaccgtgatcctgtccgtggtgcgcatcgccgcctgcttcctgtccatgatggtgaccaccatc
gtgtggaacatgatcatgctgatcctgctgccctggccctacgcccgcatccgccagggcaacctgtacggccacgtgaccggccgcatgct
gatgtggattctgggcaaccccatcaccatcgagggctccgagttctccaacacccgcgccatctacatctgcaaccacgcctccctggtgg
acatcttcctgatcatgtggctgatccccaagggcaccgtgaccatcgccaagaaggagatcatctggtatcccctgttcggccagctgtac
gtgctggccaaccaccagcgcatcgaccgctccaacccctccgccgccatcgagtccatcaaggaggtggcccgcgccgtggtgaagaag
aacctgtccctgatcatcttccccgagggcacccgctccaagaccggccgcctgctgcccttcaagaagggcttcatccacatcgccctccag
acccgcctgcccatcgtgccgatggtgctgaccggcacccacctggcctggcgcaagaactccctgcgcgtgcgccccgcccccatcaccgt
gaagtacttctcccccatcaagaccgacgactgggaggaggagaagatcaaccactacgtggagatgatccacgccctgtacgtggacc
acctgcccgagtcccagaagcccctggtgtccaagggccgcgacgcctccggccgctccaactccTGAttaattaactcgagatgtggaga
tgtagggtggtcgactcgttggaggtgggtgtttttttttatcgagtgcgcggcgcggcaaacgggtccctttttatcgaggtgttccca
acgccgcaccgccctcttaaaacaacccccaccaccacttgtcgaccttctcgtttgttatccgccacggcgccccggaggggcgtcg
tctggccgcgcgggcagctgtatcgccgcgctcgctccaatggtgtgtaatcttggaaagataataatcgatggatgaggaggaga
gcgtgggagatcagagcaaggaatatacagttggcacgaagcagcagcgtactaagctgtagcgtgttaagaaagaaaaactcg

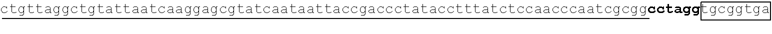

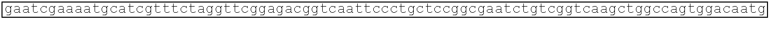

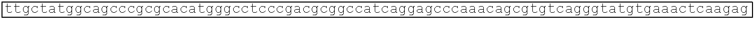

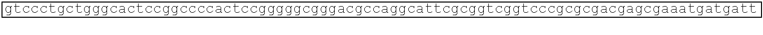

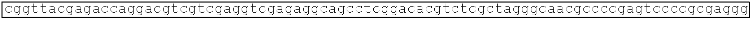

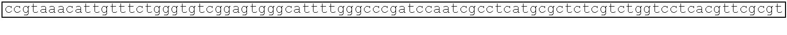

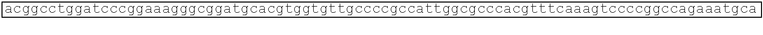

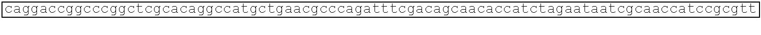

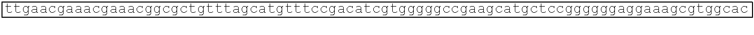

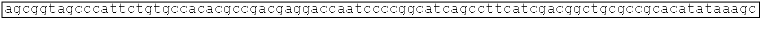

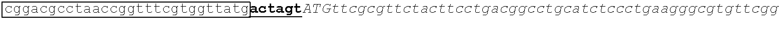

cgtctccccctcctacaacggcctgggcctgacgccccagatgggctgggacaactggaacacgttcgcctgcgacgtctccga
gcagctgctgctggacacggccgaccgcatctccgacctgggcctgaaggacatgggctacaagtacatcatcctggacgact
gctggtcctccggccgcgactccgacggcttcctggtcgccgacgagcagaagaccccaacggcatgggccacgtcgccgac
cacctgcacaacaactccttcctgacggcatgtactcctccgcgggcgagtacacgtgcgccggctaccccggctccctgggcc
gcgaggaggaggacgcccagttcttcgcgaacaaccgcgtggactacctgaagtacgacaactgctacaacaagggccagt
tcggcacgcccgagatctcctaccaccgctacaaggccatgtccgacgccctgaacaagacgggccgccccatcttctactccct
gtgcaactggggccaggacctgaccttctactggggctccggcatcgcgaactcctggcgcatgtccggcgacgtcacggcgg
agttcacgcgccccgactcccgctgcccctgcgacggcgacgagtacgactgcaagtacgccggcttccactgctccatcatga
acatcctgaacaaggccgcccccatgggccagaacgcgggcgtcggcggctggaacgacctggacaacctggaggtcggcg
tcggcaacctgacggacgacgaggagaaggcgcacttctccatgtgggccatggtgaagtcccccctgatcatcggcgcgaa
cgtgaacaacctgaaggcctcctcctactccatctactcccaggcgtccgtcatcgccatcaaccaggactccaacggcatcccc
gccacgcgcgtctggcgctactacgtgtccgacacggacgagtacggccagggcgagatccagatgtggtccggccccctgg
acaacggcgaccaggtcgtggcgctgctgaacggcggctccgtgtcccgccccatgaacacgaccctggaggagatcttcttc
gactccaacctgggctccaagaagctgacctccacctgggacatctacgacctgtgggcgaaccgcgtcgacaactccacggc
gtccgccatcctgggccgcaacaagaccgccaccggcatcctgtacaacgccaccgagcagtcctacaaggacggcctgtcca
agaacgacacccgcctgttcggccagaagatcggctccctgtcccccaacgcgatcctgaacacgaccgtccccgcccacggc
atcgcgactaccgcctgcgcccctcctccTGAtacaacttattacgtattctgaccggcgctgatgtggcgcggacgccgtcgtac
tctttcagactttactcttgaggaattgaacctttctcgcttgctggcatgtaaacattggcgcaattaattgtgtgatgaagaaaggg
tggcacaagatggatcgcgaatgtacgagatcgacaacgatggtgattgttatgaggggccaaacctggctcaatcttgtcgcatgt
ccggcgcaatgtgatccagcggcgtgactctcgcaacctggtagtgtgtgcgcaccgggtcgctttgattaaaactgatcgcattgcc
atcccgtcaactcacaagcctactctagctcccattgcgcactcgggcgcccggctcgatcaatgttctgagcggagggcgaagcgt
caggaaatcgtctcggcagctggaagcgcatggaatgcggagcggagatcgaatcagatatcAAGCTCCATCgagctccagc
cacggcaacaccgcgcgccttgcggccgagcacggcgacaagaacctgagcaagatctgcgggctgatcgccagcgacgaggg
ccggcacgagatcgcctacacgcgcatcgtggacgagttcttccgcctcgaccccgagggcgccgtcgccgcctacgccaacatga
tgcgcaagcagatcaccatgcccgcgcacctcatggacgacatgggccacggcgaggccaacccgggccgcaacctcttcgccga
cttctccgcggtcgccgagaagatcgacgtctacgacgccgaggactactgccgcatcctggagcacctcaacgcgcgctggaag
gtggacgagcgccaggtcagcggccaggccgccgcggaccaggagtacgtcctgggcctgccccagcgcttccggaaactcgcc
gagaagaccgccgccaagcgcaagcgcgtcgcgcgcaggcccgtcgccttctcctggatctccgggcgcgagatcatggtctagg
gagcgacgagtgtgcgtgcggggctggcgggagtgggacgccctcctcgctcctctctgttctgaacggaacaatcggccaccccg
cgctacgcgccacgcatcgagcaacgaagaaaaccccccgatgataggttgcggtggctgccgggatatagatccggccgcaca
tcaaagggcccctccgccagagaagaagctcctttcccagcagactcctgaagagcgtttaaac
CpauLPAAT1
SEQ ID NO: 98
ggtaccATGgccatccccgccgccgccgtgatcttcctgttcggcctgctgttcttcacctccggacctgatcatcaacctgttccaggccctgtg
cttcgtgctggtgtggcccctgtccaagaacgcctaccgccgcatcaaccgcgtgttcgccgagctgctgctgtccgagctgctgtgcctgttc
gactggtgggccggcgccaagctgaagctgttcaccgaccccgagaccttccgcctgatgggcaaggagcacgccctggtgatcatcaac
cacatgaccgagctggactggatgctgggctgggtgatgggccagcacctgggctgcctgggctccatcctgtccgtggccaagaagtcc
accaagttcctgcccgtgctgggctggtccatgtggttctccgagtacctgtacatcgagcgctcctgggccaaggaccgcaccaccctgaa
gtcccacatcgagcgcctgaccgactaccccctgcccttctggatggtgatcttcgtggagggcacccgcttcacccgcaccaagctgctggc
cgcccagcagtacgccgcctcctccggcctgcccgtgccccgcaacgtgctgatcccccgcaccaagggcttcgtgtcctgcgtgtcccacat
gcgctccttcgtgcccgccgtgtacgacgtgaccgtggccttccccaagacctcccccccccccaccctgctgaacctgttcgagggccagtcc
atcgtgctgcacgtgcacatcaagcgccacgccatgaaggacctgcccgagtccgacgacgccgtggcccagtggtgccgcgacaagttc
gtggagaaggacgccctgctggacaagcacaacgccgaggacaccttctccggccaggaggtgcaccgcaccggctcccgccccatcaa
gtccctgctggtggtgatctcctgggtggtggtgatcaccttcggcgccctgaagttcctgcagtggtcctcctggaagggcaaggccttctc
cgtgatcggcctgggcatcgtgaccctgctgatgcacatgctgatcctgtcctcccaggccgagcgctcctccaaccccgccaaggtggccc
aggccaagctgaagaccgagctgtccatctccaagaaggccaccgacaaggagaacTGActcgag
CprocLPAAT1
SEQ ID NO: 99
ggtaccATGgccatccccgccgccgccgtgatcttcctgttcggcctgatcttcttcgcctccggcctgatcatcaacctgttccag
gccctgtgcttcgtgctgatctggcccatctccaagaacgcctaccgccgcatcaaccgcgtgttcgccgagctgctgctgtccgag
ctgctgtgcctgttcgactggtgggccggcgccaagctgaagctgttcaccgaccccgagaccttccgcctgatgggcaaggagc
acgccaggtgatcatcaaccacatgaccgagaggactggatggtgggagggtgatgggccagcactteggagcctgggctc
catcctgtccgtggccaagaagtccaccaagttcctgcccgtgctgggctggtccatgtggttcaccgagtacctgtacatcgagcg
ctcctggaacaaggacaagtccaccctgaagtcccacatcgagcgcctgaaggactaccccctgcccttctggctggtgatcttcg
ccgagggcacccgatcacccagaccaagagaggccgcccagcagtacgccgcctcctccggcctgcccgtgccccgcaac
gtgctgatcccccgcaccaagggcttcgtgtcctgcgtgtcccacatgcgctccttcgtgcccgccgtgtacgacctgaccgtggcct
tccccaagacctcccccccccccaccctgctgaacctgttcgagggccagtccgtggtgctgcacgtgcacatcaagcgccacgc
catgaaggacctgcccgagtccgacgacgaggtggcccagtggtgccgcgacaagncgtggagaaggacgccagaggac
aagcacaacgccgaggacaccttaccggccaggagagcagcacaccggccgccgccccatcaagtccctgaggtggtgat
ctcctgggtggtggtgatcgccttcggcgccctgaagttcctgcagtggtcctcctggaagggcaaggccttctccgtgatcggcctg
ggcatcgtgaccagagatgcacatgagatcctgtecteccaggccgagcgaccaagcccgccaaggtggcccaggccaag
ctgaagaccgagctgtccatctccaagaccgtgaccgacaaggagaacTGActcgag
CpaiLPAAT1
SEQ ID NO: 100
ggtaccATGgccatcccctccgccgccgtggtgttcctgttcggcctgctgttcttcacctccggcctgatcatcaacctgttccagg
ccttctgcttcgtgctgatctcccccctgtccaagaacgcctaccgccgatcaaccgcgtgttcgccgagctgctgcccctggagtt
cctgtggctgaccactggtgcgccggcgccaagctgaagctgacaccgaccccgagaccaccgcctgatgggcaaggagcac
gccctggtgatcatcaaccacaagatcgagctggactggatggtgggctgggtgctgggccagcacctgggctgcctgggctcca
tcctgtccgtggccaagaagtccaccaagacctgcccgtgacggctggtccctgtggactccggctacctgacctggagcgctcc
tgggccaaggacaagatcaccctgaagtcccacatcgagtccctgaaggactaccccctgccataggctgatcatcacgtgga
gggcacccgatcacccgcaccaagctgctggccgcccagcagtacgccgcctcctccggcctgcccgtgccccgcaacgtgct
gatcccccacaccaagggcttcgtgtcctccgtgtcccacatgcgctccttcgtgcccgccatctacgacgtgaccgtggccttcccc
aagacctcccccccccccaccatgctgaagctgacgagggccagtccgtggagctgcacgtgcacatcaagcgccacgccatg
aaggacctgcccgagtccgacgacgccgtggcccagtggtgccgcgacaagacgtggagaaggacgccctgctggacaagc
acaactccgaggacaccactccggccaggaggtgcaccacgtgggccgccccatcaaggccctgctggtggtgatctcctgggt
ggtggtgatcatcacggcgccctgaagacctgctgtggtcctccctgctgtcctcctggaagggcaaggccactccgtgatcggcc
tgggcatcgtggccggcatcgtgaccctgctgatgcacatcctgatcctgtcctcccaggccgagggctccaaccccgtgaaggc
cgcccccgccaagctgaagaccgagctgtcctcctccaagaaggtgaccaacaggagaacTGActcgag
ChookLPAAT1
SEQ ID NO: 101
ggtaccATGgccatcccctccgccgccgtggtgttcctgttcggcctgctgttcttcacctccggcctgatcatcaacctgttccagg
ccttctgcttcgtgctgatctcccccctgtccaagaacgcctaccgccgcatcaaccgcgtgttcgccgagctgctgcccctggagtt
cctgtggctgaccactggtgcgccggcgccaagctgaagctgacaccgaccccgagaccaccgcctgatgggcaaggagcac
gccctggtgatcatcaaccacaagatcgagctggactggatggtgggctgggtgctgggccagcacctgggctgcctgggctcca
tcctgtccgtggccaagaagtccaccaagacctgcccgtgacggctggtccctgtggactccgagtacctgacctggagcgctcc
tgggccaaggacaagatcaccctgaagtcccacatcgagtccctgaaggactaccccctgccataggctgatcatcacgtgga
gggcacccgatcacccgcaccaagctgctggccgcccagcagtacgccgcctcctccggcctgcccgtgccccgcaacgtgct
gatcccccacaccaagggcttcgtgtcctccgtgtcccacatgcgctccttcgtgcccgccatctacgacgtgaccgtggccttcccc
aagacctcccccccccccaccatgctgaagctgacgagggccagtccgtggagctgcacgtgcacatcaagcgccacgccatg
aaggacctgcccgagtccgacgacgccgtggcccagtggtgccgcgacaagacgtggagaaggacgccctgctggacaagc
acaactccgaggacaccactccggccaggaggtgcaccacgtgggccgccccatcaaggccctgctggtggtgatctcctgggt
ggtggtgatcatcacggcgccctgaagacctgctgtggtcctccctgctgtcctcctggaagggcaaggccactccgtgatcggcc
tgggcatcgtggccggcatcgtgaccctgctgatgcacatcctgatcctgtcctcccaggccgagggctccaaccccgtgaaggc
cgcccccgccaagctgaagaccgagctgtcctcctccaagaaggtgaccaacaaggagaacTGActcgag
CignLPAAT1
SEQ ID NO: 102
ggtaccATGgccatcgccgccgccgccgtgatcttcctgttcggcctgctgttcttcgcctccggcatcatcatcaacctgttccag
gccctgtgcacgtgctgatctggcccctgtccaagaacgtgtaccgccgcatcaaccgcgtgacgccgagctgctgctgatggac
ctgctgtgcctgaccactggtgggccggcgccaagatcaagctgacaccgaccccgagaccaccgcctgatgggcatggagca
cgccctggtgatcatgaaccacaagaccgacctggactggatggtgggctggatcctgggccagcacctgggctgcctgggctc
catcctgtccatcgccaagaagtccaccaagacatccccgtgctgggctggtccgtgtggactccgagtacctgacctggagcgc
tcctgggccaaggacaagtccaccctgaagtcccacatggagaagctgaaggactaccccctgccataggctggtgatcacgt
ggagggcacccgatcacccgcaccaagctgctggccgcccagcagtacgccgcctcctccggcctgcccgtgccccgcaacgt
gctgatcccccacaccaagggcttcgtgtcctgcgtgtccaacatgcgctccttcgtgcccgccgtgtacgacgtgaccgtggcctt
ccccaagtcctcccccccccccaccatgctgaagctgacgagggccagtccatcgtgctgcacgtgcacatcaagcgccacgcc
ctgaaggacctgcccgagtccgacgacgccgtggcccagtggtgccgcgacaagacgtggagaaggacgccctgctggacaa
gcacaacgccgaggacaccactccggccaggaggtgcaccacatcggccgccccatcaagtccctgctggtggtgatcgcctg
ggtggtggtgatcatcacggcgccctgaagacctgcagtggtcctccctgctgtccacctggaagggcaaggccactccgtgatc
ggcctgggcatcgccaccctgctgatgcacatgctgatcctgtcctcccaggccgagcgctccaaccccgccaaggtggccaag
TGActcgag
CavigLPAAT1
SEQ ID NO: 103
ggtaccATGaccatcgcctccgccgccgtggtgttcctgttcggcatcctgctgttcacctccggcctgatcatcaacctgttccag
gccttctgctccgtgctggtgtggcccctgtccaagaacgcctaccgccgcatcaaccgcgtgttcgccgagttcctgcccctggag
ttcctgtggctgttccactggtgggccggcgccaagctgaagctgttcaccgaccccgagaccttccgcctgatgggcaaggagc
acgccctggtgatcatcaaccacaagatcgagctggactggatggtgggctgggtgctgggccagcacctgggctgcctgggctc
catcctgtccgtggccaagaagtccaccaagacctgcccgtgacggctggtccctgtggactccgagtacctgacctggagcgc
aactgggccaaggacaagaagaccctgaagtcccacatcgagcgcctgaaggactaccccctgccataggctgatcatcttcg
tggagggcacccgatcacccgcaccaagctgctggccgcccagcagtacgccgcctccgccggcctgcccgtgccccgcaac
gtgctgatcccccacaccaagggcttcgtgtcctccgtgtcccacatgcgctccttcgtgcccgccatctacgacgtgaccgtggcct
tccccaagacctcccccccccccaccatgctgaagctgacgagggccacttcgtggagctgcacgtgcacatcaagcgccacgc
catgaaggacctgcccgagtccgaggacgccgtggcccagtggtgccgcgacaagacgtggagaaggacgccctgctggac
aagcacaacgccgaggacaccactccggccaggaggtgcaccacgtgggccgccccatcaagtccctgctggtggtgatctcc
tgggtggtggtgatcatcacggcgccctgaagacctgcagtggtcctccctgctgtcctcctggaagggcatcgccactccgtgat
cggcctgggcaccgtggccctgctgatgcagatcctgatcctgtcctcccaggccgagcgctccatccccgccaaggagaccccc
gccaacctgaagaccgagctgtcctcctccaagaaggtgaccaacaaggagaacTGActcgag
CavigLPAAT2
SEQ ID NO: 104
ggtaccATGgccatcgccgccgccgccgtgatcgtgcccgtgtccctgctgttcttcgtgtccggcctgatcgtgaacctggtgca
ggccgtgtgatcgtgctgatccgccccctgacaagaacacctaccgccgcatcaaccgcgtggtggccgagctgctgtggctgg
agctggtgtggctgatcgactggtgggccggcgtgaagatcaaggtgacaccgaccacgagaccaccacctgatgggcaagg
agcacgccctggtgatctgcaaccacaagtccgacatcgactggctggtgggctgggtgctggcccagcgctccggctgcctggg
ctccaccctggccgtgatgaagaagtcctccaagacctgcccgtgatcggctggtccatgtggactccgagtacctgacctggag
cgcaactgggccaaggacgagtccaccctgaagtccggcctgaaccgcctgaaggactaccccctgccataggctggccctgt
tcgtggagggcacccgatcacccgcgccaagctgctggccgcccagcagtacgccgcctcctccggcctgcccgtgccccgca
acgtgctgatcccccgcaccaagggcttcgtgtcctccgtgtcccacatgcgctccttcgtgcccgccatctacgacgtgaccgtgg
ccatccccaagacctcccccccccccaccctgctgcgcatgacaagggccagtcctccgtgctgcacgtgcacctgaagcgcca
ccagatgaacgacctgcccgagtccgacgacgccgtggcccagtggtgccgcgacatcacgtggagaaggacgccctgctgg
acaagcacaacgccgaggacaccactccggccaggagctgcaggacaccggccgccccatcaagtccctgctgatcgtgatct
cctgggccgtgctggtggtgacggcgccgtgaagacctgcagtggtcctccctgctgtcctcctggaagggcctggccactccgg
catcggcctgggcgtgatcaccctgctgatgcacatcctgatcctgactcccagtccgagcgctccacccccgccaaggtggccc
ccgccaagcccaagatcgagggcgagtcctccaagaccgagatggagaaggagcacTGActcgag
CpalLPAAT1
SEQ ID NO: 105
ggtaccATGgccatcgccgccgccgccgtgatcgtgcccctgggcctgctgttcttcgtgtccggcctgatcgtgaacctggtgca
ggccgtgtgcttcgtgctgatccgccccctgtccaagaacacctaccgccgcatcaaccgcgtggtggccgagctgctgtggctgg
agctggtgtggctgatcgactggtgggccggcgtgaagatcaaggtgttcaccgaccacgagaccctgtccctgatgggcaagg
agcacgccctggtgatctgcaaccacaagtccgacatcgactggctggtgggctgggtgctggcccagcgctccggctgcctggg
ctccaccctggccgtgatgaagaagtcctccaagttcctgcccgtgatcggctggtccatgtggttctccgagtacctgcccgagtcc
gacgacgccgtggcccagtggtgccgcgacatcttcgtggagaaggacgccctgctggacaagcacaacgccgaggacacctt
ctccggccaggagctgcaggacaccggccgccccatcaagtccctgctggtggtgatctcctgggccgtgctggtgatcttcggcg
ccgtgaagttcctgcagtggtcctccctgctgtcctcctggaagggcctggccttctccggcgtgggcctgggcatcatcaccctgct
gatgcacatcctgatcctgttctcccagtccgagcgctccacccccgccaaggtggcccccgccaagccaagaaggacggcga
gtcctccaagaccgagatcgagaaggagaacgttcctggagcgctcctgggccaaggacgagaacaccctgaagtccggcct
gaaccgcctgaaggactaccccctgcccUctggctggccctgttcgtggagggcacccgcttcacccgcgccaagctgctggcc
gcccagcagtacgccacctcctccggcctgcccgtgccccgcaacgtgctgatcccccgcaccaagggcttcgtgtcctccgtgtc
ccacatgcgctcatcgtgcccgccatctacgacgtgaccgtggccatccccaagacctcccccccccccaccatgctgcgcatgtt
caagggccagtcctccgtgctgcacgtgcacctgaagcgccacctgatgaaggacctTGActcgag
CuPSR23 LPAAT2
SEQ ID NO: 106
ggtaccATGgccatcgccgccgccgccgtgatcttcctgttcggcctgatcttcttcgcctccggcctgatcatcaacctgttccag
gccctgtgcttcgtgctgatccgccccctgtccaagaacgcctaccgccgcatcaaccgcgtgttcgccgagctgctgctgtccgag
ctgctgtgcctgttcgactggtgggccggcgccaagctgaagctgttcaccgaccccgagaccttccgcctgatgggcaaggagc
acgccctggtgatcatcaaccacatgaccgagctggactggatggtgggctgggtgatgggccagcacttcggctgcctgggctc
catcatctccgtggccaagaagtccaccaagttcctgcccgtgctgggctggtccatgtggttctccgagtacctgtacctggagcg
ctcctgggccaaggacaagtccaccctgaagtcccacatcgagcgcctgatcgactaccccctgcccttctggctggtgatcttcgt
ggagggcacccgcttcacccgcaccaagctgctggccgcccagcagtacgccgtgtcctccggcctgcccgtgccccgcaacgt
gctgatcccccgcaccaagggcttcgtgtcctgcgtgtcccacatgcgctcatcgtgcccgccgtgtacgacgtgaccgtggccttc
cccaagacctcccccccccccaccctgctgaacctgttcgagggccagtccatcatgctgcacgtgcacatcaagcgccacgcca
tgaaggacctgcccgagtccgacgacgccgtggccgagtggtgccgcgacaagttcgtggagaaggacgccctgctggacaa
gcacaacgccgaggacaccttctccggccaggaggtgtgccactccggctcccgccagctgaagtccctgctggtggtgatctcc
tgggtggtggtgaccaccttcggcgccctgaagttcctgcagtggtcctcctggaagggcaaggccttctccgccatcggcctggg
catcgtgaccctgctgatgcacgtgctgatcctgtcctcccaggccgagcgctccaaccccgccgaggtggcccaggccaagctg
aagaccggcctgtccatctccaagaaggtgaccgacaaggagaacTGActcgag
CkoeLPAAT1
SEQ ID NO: 107
ggtaccATGgccatccccgccgccgtggccgtgatccccatcggcctgctgttcatcatctccggcctgatcgtgaacctgatcca
ggccgtggtgtacgtgctgatccgccccctgtccaagaacctgcaccgcaagatcaacaagcccatcgccgagctgctgtggctg
gagctgatctggctggtggactggtgggccggcatcaaggtggaggtgtacgccgactcccagaccctggagctgatgggcaag
gagcacgccctgctgatctgcaaccaccgctccgacatcgactggctggtgggctgggtgctggcccagcgcgcccgctgcctgg
gctccgccctggccatcatgaagaagtccgccaagttcctgcccgtgatcggctggtccatgtggttctccgactacatcttcctgga
ccgcacctgggccaaggacgagaagaccctgaagtccggcttcgagcgcctggccgacttccccatgcccttctggctggccctg
ttcgtggagggcacccgcttcaccaaggccaagctgctggccgcccaggagtacgccgcctcccgcggcctgcccgtgccccag
aacgtgctgatcccccgcaccaagggcttcgtgaccgccgtgacccacatgcgctcctacgtgcccgccatctacgactgcaccg
tggacatctccaaggcccaccccgccccctccatcctgcgcctgatccgcggccagtcctccgtggtgaaggtgcagatcacccg
ccactccatgcaggagctgcccgagaccgccgacggcatctcccagtggtgcatggacctgttcgtgaccaaggacggcttcctg
gagaagtaccactccaaggacatcttcggctccctgcccgtgcagaacatcggccgccccgtgaagtccctgatcgtggtgctgtg
ctggtactgcctgatggccttcggcctgttcaagttcttcatgtggtcctccctgctgtcctcctgggagggcatcctgtccctgggcctg
atcctgctggccgtggccatcgtgatgcagatcctgatccagtccaccgagtccgagcgctccacccccgtgaagtccatccaga
aggacccctccaaggagaccctgctgcagaacTGActcgag
CkoeLPAAT2
SEQ ID NO: 108
ggtaccATGcacgtgctgctggagatggtgaccttccgcttctcctccttcttcgtgttcgacaacgtgcaggccctgtgcttcgtgct
gatctggcccctgtccaagtccgcctaccgcaagatcaaccgcgtgttcgccgagctgctgctgtccgagctgctgtgcctgttcga
ctggtgggccggcgccaagctgaagctgttcaccgaccccgagaccttccgcctgatgggcaaggagcacgccctggtgatcac
caaccacaagatcgacctggactggatgatcggctggatcctgggccagcacttcggctgcctgggctccgtgatctccatcgcca
agaagtccaccaagttcctgcccatchcggctggtccctgtggttctccgagtacctgttcctggagcgcaactgggccaaggaca
agcgcaccctgaagtcccacatcgagcgcatgaaggactaccccctgcccctgtggctgatcctgttcgtggagggcacccgctt
cacccgcaccaagctgctggccgcccagcagtacgccgcctcctccggcctgcccgtgccccgcaacgtgctgatcccccacac
caagggcttcgtgtcctccgtgtcccacatgcgctcatcgtgcccgccgtgtacgacgtgaccgtggcchccccaagacctcccc
cccccccaccatgctgtccctgttcgagggccagtccgtggtgctgcacgtgcacatcaagcgccacgccatgaaggacctgccc
gactccgacgacgccgtggcccagtggtgccgcgacaagttcgtggagaaggacgccctgctggacaagcacaacgccgagg
acaccttctccggccaggaggtgcaccacgtgggccgccccatcaagtccctgctggtggtgatctcctggatggtggtgatcatct
tcggcgccctgaagttcctgcagtggtcctccctgctgtcctcctggaagggcaaggccttctccgccatcggcctgggcatcgcca
ccctgctgatgcacgtgctggtggtgttctcccaggccgaccgctccaaccccgccaaggtgccccccgccaagctgaacaccga
gctgtcctcctccaagaaggtgaccaacaaggagaacTGActcgag
CprocLPAAT2
SEQ ID NO: 109
ggtaccATGgccatccccgccgccgtggccgtgatccccatcggcctgctgttcatcatctccggcctgatcgtgaacctgatcca
ggccgtggtgtacgtgctgatccgccccctgtccaagaacctgtaccgcaagatcaacaagcccatcgccgagctgctgtggctg
gagctgatctggctggtggactggtgggccggcatcaaggtggaggtgtacgccgactccgagaccctggagtccatgggcaag
gagcacgccctgctgatctgcaaccaccgctccgacatcgactggctggtgggctgggtgctggcccagcgcgcccgctgcctgg
gctccgccctggccatcatgaagaagtccgccaagttcctgcccgtgatcggctggtccatgtggttctccgactacatcttcctgga
ccgcacctgggagaaggacgagaagaccctgaagtccggcttcgagcgcctggccgachccccatgcccttctggctggccct
gttcgtggagggcacccgcttcaccaaggccaagctgctggccgcccaggagttcgccgcctcccgcggcctgcccgtgcccca
gaacgtgctgatcccccgcaccaagggcttcgtgaccgccgtgacccacatgcgctcctacgtgcccgccatctacgactgcacc
gtggacatctccaaggcccaccccgccccctccatcctgcgcctgatccgcggccagtcctccgtggtgaaggtgcagatcaccc
gccactccatgcaggagctgcccgagacccccgacggcatctcccagtggtgcatggacctgttcgtgaccaaggacgcttcct
ggagaagtaccactccaaggacatcttcggctccctgcccgtgcacgacatcggccgccccgtgaagtccctgatcgtggtgctgt
gctggtactccctgatggcchcggcactacaagttcttcatgtggtcctccctgctgtcctcctgggagggcatcctgtccctgggcct
ggtgctgatcgtgatcgccatcgtgatgcagatcctgatccagtcctccgagtccgagcgctccacccccgtgaagtccgtgcaga
aggacccctccaaggagaccctgctgcagaacTGActcgag
CavigGPAT9
SEQ ID NO: 110
ggtaccATGgccaccggcggctccctgaagccctcctcctccgacctggacctggaccaccccaacatcgaggactacctgcc
ctccggctcctccatcaacgagcccgccggcaagctgcgcctgcgcgacctgctggacatctcccccaccctgaccgaggccgc
cggcgccatcgtggacgactcchcacccgctgatcaagtccatcccccgcgagccctggaactggaacctgtacctgttccccct
gtggtgcatcggcgtgctgatccgctacttcatcctgttccccggccgcgtgatcgtgctgaccatgggctggatcaccgtgatctcct
catcatcgccgtgcgcgtgctgctgaagggccacgacgccctgcagatcaagctggagcgcctgatcgtgcagctgctgtgctcc
tcatcgtggcctcctggaccggcgtggtgaagtaccacggcccccgcccctccatccgccccaagcaggtgtacgtggccaacc
acacctccatgatcgacttchcatcctggaccagatgaccgtgttctccgtgatcatgcagaagcaccccggctgggtgggcctgc
tgcagtccaccctgctggagtccgtgggctgcatctggacgaccgcgccgaggccaaggaccgcggcatcgtggccaagaagc
tgtgggaccacgtgcacggcgagggcaacaaccccctgctgatchccccgagggcacctgcgtgaacaacaactactccgtga
tgttcaagaagggcgcchcgagctgggctgcaccgtgtgccccgtggccatcaagtacaacaagatchcgtggacgcatctgg
aactccaagaagcagtcchcacccgccacctgctgcagctgatgacctcctgggccgtggtgtgcgacgtgtggtacttggagcc
ccagaccctgaagcccggcgagacccccatcgagttcgccgagcgcgtgcgcgacatcatctccgcccgcgccggcctgaaga
aggtgccctgggacggctacctgaagtactcccgcccctcccccaagcaccgcgagcgcaagcagcagacchcgccgagtcc
gtgctgcagcgcctggaggagTGActcgag
ChookGPAT9-1
SEQ ID NO: 111
ggtaccATGgccaccgccggctccctgaagccctcccgctccgagctggacttcgaccgccccaacatcgaggactacctgcc
ctccggctcctccatcatcgagcccgccggcaagctgcgcctgcgcgacctgctggacatctcccccaccctgaccgaggccgcc
ggcgccatcgtggacgactcatcacccgctgatcaagtccaacccccccgagccctggaactggaacatctacctgttccccct
gtggtgatcggcgtgctgatccgctacctgatcctgttccccgcccgcgtgatcgtgctgaccatcggctggatcatatcctgtcctc
atcatccccgtgcacctgctgctgaagggccacgacgccctgcgcatcaagctggagcgcctgctggtggagctgatctgctcat
atcgtggcctcctggaccggcgtggtgaagtaccacggcccccgcccctccatccgccccaagcaggtgtacgtggccaaccac
acctccatgatcgacttatcatcctggaccagatgaccgtgttctccgtgatcatgcagaagcaccccggctgggtgggcctgctg
cagtccaccctgctggagtccgtgggctgcatctggttcgaccgcgccgaggccaaggaccgcggcatcgtggccaagaagctg
tgggaccacgtgcacggcgagggcaacaaccccctgctgatatccccgagggcacctgcgtgaacaacaactactccgtgatg
ttcaagaagggcgcatcgagctgggctgcaccgtgtgccccgtggccatcaagtacaacaagatatcgtggacgcatctggaa
ctccaagaagcagtcatcacccgccacctgctgcagctgatgacctcctgggccgtggtgtgcgacgtgtggtacttggagcccc
agaccctgaagcccggcgagacccccatcgagttcgccgagcgcgtgcgcgacatcatctccgtgcgcgccggcctgaagaag
gtgccctgggacggctacctgaagtactcccgcccctcccccaagcacaccgagcgcaagcagcagaacttcgccgagtccgt
gctgcagcgcctggagaagaagTGActcgag
CignGPAT9-1
SEQ ID NO: 112
ggtaccATGgccaccggcggccgcctgaagccctcctcctccgagctggacctggaccgcgccaacaccgaggactacctgc
cctccggctcctccatcaacgagcccgtgggcaagctgcgcctgcgcgacctgctggacatctcccccaccctgaccgaggccg
ccggcgccatcgtggacgactcatcacccgctgatcaagtccatcccccccgagccctggaactggaacatctacctgttccccc
tgtggtgatcggcgtgctgatccgctacttcatcctgttccccgcccgcgtgatcgtgctgaccatcggctggatcaccgtgatctcct
catcaccgccgtgcgatcctgctgaagggccacaacgccctgcagatcaagctggagcgcctgatcgtgcagctgctgtgctcc
tcatcgtggcctcctggaccggcgtggtgaagtaccacggcccccgcccctccatccgccccaagcaggtgtacgtggccaacc
acacctccatgatcgacttcctgatcctggaccagatgaccgtgttctccgtgatcatgcagaagcaccccggctgggtgggcctg
ctgcagtccaccctgctggagtccgtgggctgcatctggttcaaccgcgccgaggccaaggaccgcgagatcgtggccaagaag
ctgtgggaccacgtgcacggcgagggcaacaaccccctgctgatatccccgagggcacctgcgtgaacaaccactactccgtg
atgttcaagaagggcgcatcgagctgggctgcaccgtgtgccccgtggccatcaagtacaacaagatatcgtggacgcatctg
gaactcccgcaagcagtcatcaccatgcacctgctgcagctgatgacctcctgggccgtggtgtgcgacgtgtggtacttggagc
cccagaccctgaagcccggcgagaccgccatcgagttcgccgagcgcgtgcgcgacatcatctccgtgcgcgccggcctgaag
aaggtgccctgggacggctacctgaagtactcccgcccctcccccaagcaccgcgagtccaagcagcagtcatcgccgagtcc
gtgctgcgccgcctggaggagaagTGActcgag
CignGPAT9-2
SEQ ID NO: 113
ggtaccATGgccaccggcggccgcctgaagccctcctcctccgagctggacctggaccgcgccaacaccgaggactacctgc
cctccggctcctccatcaacgagcccgtgggcaagctgcgcctgcgcgacctgctggacatctcccccaccctgaccgaggccg
ccggcgccatcgtggacgactcatcacccgctgatcaagtccatcccccccgagccctggaactggaacatctacctgttccccc
tgtggtgatcggcgtgctgatccgctacttcatcctgttccccgcccgcgtgatcgtgctgaccatcggctggatcaccgtgatctcct
catcaccgccgtgcgatcctgctgaagggccacaacgccctgcagatcaagctggagcgcctgatcgtgcagctgctgtgctcc
tcatcgtggcctcctggaccggcgtggtgaagtaccacggcccccgcccctccatccgccccaagcaggtgtacgtggccaacc
acacctccatgatcgacttcctgatcctggaccagatgaccgtgttctccgtgatcatgcagaagcaccccggctgggtgggcctg
ctgcagtccaccctgctggagtccgtgggctgcatctggttcaaccgcgccgaggccaaggaccgcgagatcgtggccaagaag
ctgtgggaccacgtgcacggcgagggcaacaaccccctgctgatatccccgagggcacctgcgtgaacaaccactactccgtg
atgttcaagaagggcgcatcgagctgggctgcaccgtgtgccccgtggccatcaagtacaacaagatatcgtggacgcatctg
gaactccaagaagcactcatcacccgccacctgctgcagctgatgacctcctgggccgtggtgtgcgacgtgtggtacttggagc
cccagaccctgaagcccggcgagacccccatcgagttcgccgagcgcgtgcgcgacatcatctccgtgcgcgccgacctgaag
aaggtgccctgggacggctacctgaagtactcccgcccctcccccaagcaccgcgagcgcaagcagcagaagttcgccgagtc
cgtgctgcgccgcctggaggagaagTGActcgag
CpalGPAT9-1
SEQ ID NO: 114
ggtaccATGgccaccgccggccgcctgaagccctcctcctccgagctggagctggacctggaccgcccaacatcgaggact
acctgccctccggctcctccatcaacgagcccgccggcaagctgcgcctgcgcgacctgctggacatctcccccatgctgaccga
ggccgccggcgccatcgtggacgactccacacccgctgatcaagtccatcccccccgagccctggaactggaacatctacctgt
tccccctgtggtgatcggcgtgctgatccgctacctgatcctgaccccgcccgcgtgatcgtgctgaccgtgggctggatcaccgtg
atctcctccacatcaccgtgcgcacctgctgaagggccacgactccctgcgcatcaagctggagcgcctgatcgtgcagctgttct
gctcctccacgtggcctcctggaccggcgtggtgaagtaccacggcccccgcccctccatccgcccccagcaggtgtacgtggcc
aaccacacctccatgatcgacttcatcatcctgaaccagatgaccgtgactccgccatcatgcagaagcaccccggctgggtggg
cctgatccagtccaccatcctggagtccgtgggctgcatctggacaaccgcgccgaggccaaggaccgcgagatcgtggccaa
gaagctgctggaccacgtgcacggcgagggcaacaaccccctgctgatcaccccgagggcacctgcgtgaacaaccactactc
cgtgatgacaagaagggcgccacgagctgggctgcaccgtgtgccccgtggccatcaagtacaacaagatcacgtggacgcct
tctggaactccaagaagcagtccacaccatgcacctgctgcagctgatgacctcctgggccgtggtgtgcgacgtgtggtacagg
agccccagaccctgaagcccggcgagacccccatcgagacgccgagcgcgtgcgcgacatcatctccgtgcgcgccggcctg
aagaaggtgccctgggacggctacctgaagtactcccgcccctcccccaagcaccgcgagcgcaagcagcagtccacgccga
gtccgtgctgcgccgcctggagaagcgcTGActcgag
CpalGPATt9-2
SEQ ID NO: 115
ggtaccATGgccaccgccggccgcctgaagccctcctcctccgagctggagctggacctggaccgccccaacatcgaggact
acctgccctccggctcctccatcaacgagcccgccggcaagctgcgcctgcgcgacctgctggacatctcccccatgctgaccga
ggccgccggcgccatcgtggacgactccacacccgctgatcaagtccatcccccccgagccctggaactggaacatctacctgt
tccccctgtggtgatcggcgtgctgatccgctacctgatcctgaccccgcccgcgtgatcgtgctgaccgtgggctggatcaccgtg
atctcctccacatcaccgtgcgcacctgctgaagggccacgactccctgcgcatcaagctggagcgcctgatcgtgcagctgttct
gctcctccacgtggcctcctggaccggcgtggtgaagtaccacggcccccgcccctccatccgcccccagcaggtgtacgtggcc
aaccacacctccatgatcgacttcatcatcctgaaccagatgaccgtgactccgccatcatgcagaagcaccccggctgggtggg
cctgatccagtccaccatcctggagtccgtgggctgcatctggacaaccgcgccgaggccaaggaccgcgagatcgtggccaa
gaagctgctggaccacgtgcacggcgagggcaacaaccccctgctgatcaccccgagggcacctgcgtgaacaaccactactc
cgtgatgacaagaagggcgccacgagctgggctgcaccgtgtgccccgtggccatcaagtacaacaagatcacgtggacgcct
tctggaactccaagaagctgtcatcaccatgcacctgctgcagctgatgacctcctgggccgtggtgtgcgacgtgtggtacagg
agccccagaccctgaagcccggcgagacccccatcgagacgccgagcgcgtgcgcgacatcatctccgtgcgcgccggcctg
aagaaggtgccctgggacggctacctgaagtactcccgcccctcccccaagcaccgcgagcgcaagcagcagaccacgccg
agtccgtgctgcgccgcctggaggagaagggcaacgtggtgcccaccgtgaacTGActcgag
CavigDGAT1
SEQ ID NO: 116
ggtaccATGgccatcgccgacggcggcatcatcggcgccgccggctccatctccgccctgaccgccgacaccgaccccccct
ccctgcgccgccgcaacgtgcccgccggccaggcctccgccgtgtccgccactccaccgagtccatggccaagcacctgtgcga
cccctcccgcgagccctccccctcccccaagtcctccgacgacggcaaggaccccgacatcggctccgtggactccctgaacga
gaagccctcctcccccgccgccggcaagggccgcctgcagcacgacctgcgcttcacctaccgcgcctcctcccccgcccaccg
caaggtgaaggagtcccccctgtcctcctccaacatcacaagcagtcccacgccggcctgacaacctgtgcgtggtggtgctggt
ggccgtgaactcccgcctgatcatcgagaacctgatgaagtacggcctgctgatcaagaccggataggactcctcccgctccct
gcgcgactggcccctgacatgtgctgcctgtccctgcccatcaccccctggccgccacctggtggagaagctggcccagaagaa
ccgcctgcaggagcccaccgtggtgtgctgccacgtgctgatcacctccgtgtccatcctgtaccccgtgctggtgatcctgcgctg
cgactccgccgtgctgtccggcgtggccctgatgctgacgcctgcatcgtgtggctgaagctggtgtcctacgcccactccaactac
gacatgcgctacgtggccaagtccctggacaagggcgagcccgtggtggactccgtgatcgccgaccacccctaccgcgtgga
ctacaaggacctggtgtacttcatggtggcccccaccctgtgctaccagctgtcctaccccctgaccccctgcgtgcgcaagtcctg
gatcgcccgccaggtgatgaagctggtgctgacaccggcgtgatgggatcatcgtggagcagtacatcaaccccatcgtgcag
aactccaagcaccccctgaagggcgacctgctgtacgccatcgagcgcgtgctgaagctgtccgtgcccaacctgtacgtgtggc
tgtgcatgactactgatcaccacctgtggctgaacatcctggccgagctgatctgatcggcgaccgcgagactacaaggactgg
tggaacgccaagaccgtggaggagtactggcgcatgtggaacatgcccgtgcacaagtggatggtgcgccacatctacacccct
gcctgcgcaacggcatcccccgcggcgtggccgtgctgatcgccacctggtgtccgccgtgaccacgagctgtgcatcgccgtgc
cctgccacgtgttcaagctgtgggcchcatcggcatcatgttccaggtgcccctggtgctggtgtccaactgcctgcagaagaagtt
ccagtcctccatggccggcaacatgttatctgghcatchctgcatchcggccagcccatgtgcgtgctgctgtactaccacgacct
gatgaaccgcaagggctccgcatcgacTGActcgag
ChookDGAT1-1
SEQ ID NO: 117
ggtaccATGgccatcgccgacggcggctccgccggcgccgccggctccatctccggctccgacccctccccctccaccgcccc
ctccctgcgccgccgcaacgcctccgccggccaggcchaccaccgagtccatggcccgcgacctgtgcgacccctcccgcga
gccctccctgtcccccaagtcctccgacgacggcaaggaccccgccgacgacatcggcgccgccgactccgtggactccggcg
gcgtgaaggacgagaagccctcctcccaggccgccgccaaggcccgcctggagcacgacctgcgatcacctaccgcgcctcc
tcccccgcccaccgcaaggtgaaggagtcccccctgtcctcctccaacatchcaagcagtcccacgccggcctgttcaacctgtg
cgtgggtggtgctggtggccgtgaactcccgcctgatcatcgagaacctgatgagtacggcctgctgataagaccggcttctggtt
ctcctcccgctccctgcgcgactggcccctgttcatgtgctgcctgtccctgcccatcaccccctggccgcchcctggtggagaagc
tggcccagaagaaccgcctgcaggagcccaccgtggtgtgctgccacgtgatcatcacctccgtgtccatcctgtaccccgtgctg
gtgatcctgcgctgcgactccgccgtgctgtccggcgtggccctgatgctgttcgcctgcatcgtgtggctgaagctggtgtcctacg
cccacgccaactacgacatgcgctccgtggccaagtccctggacaagggcgagaccgtggccgactccgtgatcgtggaccac
ccctaccgcgtggactacaaggacctggtgtacttcatggtggcccccaccctgtgctaccagctgtcctaccccctgaccccctac
gtgcgcaagtcctgggtggcccgccaggtgatgaagctggtgctgttcaccggcgtgatgggchcatcgtggagcagtacatcaa
ccccatcgtgcagaactccaagcaccccctgaagggcgacctgctgtacgccatcgagcgcgtgctgaagctgtccgtgcccaa
cctgtacgtgtggctgtgcatgttctactgatchccacctgtggctgaacatcctggccgagctgacctgatcggcgaccgcgagt
tctacaaggactggtggaacgccaagaccgtggaggagtactggcgcatgtggaacatgcccgtgcacaagtggatggtgcgc
cacatctacttcccctgcctgcgcaacggcatcccccgcggcgtggccgtgctgatcgcchcctggtgtccgccgtgttccacgag
ctgtgcatcgccgtgccctgccacgtgttcaagctgtgggcchcatcggcatcatgttccaggtgcccctggtgctggtgtccaactg
cctgcagaagaagttccagtcctccatggccggcaacatgttatctgghcatchctgcatchcggccagcccatgtgcgtgctgct
gtactaccacgacctgatgaaccgaagggctcccgcatcgacTGActcgag
CavigLPCAT
SEQ ID NO: 118
ggtaccATGggcctggtgtccgtggccgccgccatcggcgtgtccgtgcccgtggcccgcttcctgctgtgcttcctggccaccat
ccccgtgtcchcctgtggcgcctggtgcccggccgcctgcccaagcacctgtactccgccgcctccggcgccatcctgtcctacct
gtcatcggcgcctcctccaacctgcacttcatcgtgcccatgaccctgggctacctgtccatgctgttchccgcccatctccggcct
gctgaccttcttcctgggcttcggctacctgatcggctgccacgtgtactacatgtccggcgacgcctggaaggagggcggcatcg
acgccaccggcgccctgatggtgctgaccctgaaggtgatctcctgctccatgaactacaacgacggcctgctgaaggaggagg
gcctgcgcgagtcccagaagaagaaccgcctgaccaagatgccctccctgatcgagtacttcggctactgcctgtgctgcggctc
ccacttcgccggccccgtgtacgagatgaaggactacctggagtggaccgagggcaagggcatctggtcccgctcccagaagg
agcccaagccctccccatcggcggcgccctgcgcgccatcatccaggccgccgtgtgcatggccatgtacctgtacctggtgccc
caccaccccctgacccgatcaccgagcccgtgtactacgagtggggcttchccgccgcctgtcctaccagtacatggccgccctg
accgcccgctggaagtactacttcatctggtccatctccgaggcctccctgatcatctccggcctgggcttctccggctggaccgagt
cctccccccccaagccccgctgggaccgcgccaagaacgtggacatcatcggcgtggagttcgccaagtcctccgtgcagctgc
ccctggtgtggaacatccaggtgtccatctggctgcgccactacgtgtacgaccgcctggtgcagaacggcaagcgccccggat
caccagctgctggccacccagaccgtgtccgccgtgtggcacggcctgtaccccggctacatcatchatcgtgcagtccgccctg
atgatcgccggctcccgcgtgatctaccgctggcagcaggcggtgcccccaagatgggcctggtgaagaacatcttcgtgttctt
caacttcgcctacaccctgctggtgctgaactactccgccgtgggchcatggtgctgtccatgcacgagaccctggcctcctacgg
ctccgtgtactacatcggcaccatcctgcccatcaccctgatcctgctgtcctacgtgatcaagcccggcaagcccgcccgctccaa
ggcccacaaggagcagTGActcgag
CpalLPCAT
SEQ ID NO: 119
ggtaccATGgagctgggctccgtggccgccgccatcggcgtgtccgtgcccgtggcccgcttcctgctgtgcttcctggccaccat
ccccgtgtcchcctgtggcgcctggtgcccggccgcctgcccaagcacctgtactccgccgcctccggcgccatcctgtcctacct
gtccttcggcccctcctccaacctgcacttcatcgtgcccatgaccctgggctacctgtccatgctgttcttccgccccttctcggcct
gctgaccttcttcctgggcttcggctacctgatcggctgccacgtgtactacatgtccggcgacgcctggaaggagggcggcatcg
acgccaccggcgccctgatggtgctgaccctgaaggtgatctcctgctccatcaactacaacgacggcctgctgaaggaggagg
gcctgcgcgagtcccagaagaagaaccgcctgaccaagatgccctccctgatcgagtacatcggctactgcctgtgctgcggctc
ccacttcgccggccccgtgtacgagatgaaggactacctggagtggaccgagggcaagggcgtgtggtcccactccgagaagg
agcccaagccctccccatcggcggcgccctgcgcgccatcatccaggccgccgtgtgcatggccatgtacatgtacctggtgccc
caccaccccctgtcccgatcaccgagcccgtgtactacgagtggggcacttccgccgcctgtcctaccagtacatggccggcctg
accgcccgctggaagtactacttcatctggtccatctccgaggcctccctgatcatctccggcctgggcttctccggctggaccgagt
cctccccccccaagccccgctgggaccgcgccaagaacgtggacatcatcggcgtggagttcgccaagtcctccgtgcagctgc
ccctggtgtggaacatccaggtgtccacctggctgcgccactacgtgtacgaccgcctggtgcagaacggcaagcgccccggat
caccagctgctggccacccagaccgtgtccgccatctggcacggcctgtaccccggctacatcatatatcgtgcagtccgccctg
atgatcgccggctcccgcgtgatctaccgctggcagcaggccgtgccccccaagatgggcctggtgaagaacatcttcgtgttctt
caacttcgcctacaccctgctggtgctgaactactccgccgtgggatcatggtgctgtccatgcacgagaccctggcctcctacgg
ctccgtgtactacatcggcaccatcctgcccatcaccctgatcctgctgtcctacgtgatcaagcccggcaagcccgcccgctccaa
ggcccacaaggagcagTGActcgag
CpauLPCAT
SEQ ID NO: 120
ggtaccATGgagctggagatcggctccgtggccgccgccatcggcgtgtccgtgcccgtggcccgcttcctgctgtgcttcctgg
ccaccatccccgtgtccttcctgtgccgcctgctgcccgcccgcctgcccaagcacctgtactccgccgcctccggcgccatcctgt
cctacctgtccttcggcccctcctccaacctgcacttcatcgtgcccatgtccctgggctacctgtccatgctgttcttccgccccttctcc
ggcctgctgaccttcttcctgggcttcggctacctgatcggctgccacgtgtactacatgtccggcgacgcctggaaggagggcgg
catcgacgccaccggcgccctgatggtgctgaccctgaaggtgatctcctgctccatcaactacaacgacggcctgctgaaggag
gagggcctgcgcgagtcccagaagaagaaccgcctgaccaagatgccctccctgatcgagtacttcggctactgcctgtgctgcg
gctcccacttcgccggccccgtgtacgagatgaaggactacctggagtggaccgagggcaagggcatctggtcccgctccgaga
aggaccccaagccctccccatcggcggcgccctgcgcgccatcatccaggccgccgtgtgcatggccatgcacatgtacctggt
gccccaccaccccctgacccgcttcaccgagcccgtgtactacgagtggggcttcttccgccgcctgtcctaccagtacatggccg
cccagaccgcccgctggaagtactacttcatctggtccatctccgaggcctccctgatcatctccggcctgggcttctccggctggac
cgagtcctccccccccaagccccgctgggacaaggccaagaacgtggacatcatcggcgtggagttcgccaagtcctccgtgca
gctgcccctggtgtggaacatccaggtgtccacctggctgcgccactacgtgtacgaccgcctggtgcagaacggcaagcgccc
cggcttcttccagctgctggccacccagaccgtgtccgccgtgtggcacggcctgtaccccggctacatcatcttcttcgtgcagtcc
gccctgatgatcgccggctcccgcgtgatctaccgctggcagcaggccgtgccccagaagatgggcctggtgaagaacatcttcg
tgttcttcaacttcgcctacaccctgctggtgctgaactactccgccgtgggcttcatggtgctgtccatgcacgagaccctggcctcc
tacggctccgtgtactacatcggcaccatcctgcccatcaccctgatcctgctgtcctacgtgatcaagcccggcaagcccacccg
ctccaaggtgcacaaggagcagTGActcgag
CschuLPCAT
SEQ ID NO: 121
ggtaccATGgagctggagatggagcccctggccgccgccatcggcgtgtccgtggccgtgttccgcttcctggtgtgcttcatcg
ccaccatccccgtgtccttcatctgccgcctggtgcccggcggcctgccccgccacctgttctccgccgcctccggcgccgtgctgtc
ctacctgtccttcggcttctcctccaacctgcacttcctggtgcccatgaccctgggctacctgtccatgatcctgttccgccgcttctgc
ggcatcctgaccttcttcctgggcttcggctacctgatcggctgccacgtgtactacatgtccggcgacgcctggaaggagggcgg
catcgacgccaccggcgccctgatggtgctgaccctgaaggtgatctcctgctccatcaactacaacgacggcctgctgaaggag
gagggcctgcgcgagtcccagaagaagaaccgcctgatccgcctgccctccctgatcgagtacttcggctactgcctgtgctgcg
gctcccacttcgccggccccgtgtacgagatgaaggactacctggactggaccgagggcaagggcatctggtcccactccgaga
agggccccaagccctcccccctgcgcgccgccctgcgcgccatcatccaggccggcttctgcatggccatgtacctgtacctggtg
ccccactaccccctgacccgcttcaccgaccccgtgtactacgagtggggcatcctgcgccgcctgtcctaccagtacatggcctc
cttcaccgcccgctggaagtactacttcatctggtccatctccgaggcctccctgatcatctccggcctgggcttctccggctggacc
gagtcctccccccccaagccccgctgggaccgcgccaagaacgtggacatcctgggcgtggagctggccaagtcctccgtgca
gatccccctggtgtggaacatccaggtgtccacctggctgcgccactacgtgtacgaccgcctggtgcagaacggcaagcgccc
cggatcctgcagctgctggccacccagaccgtgtccgccatctggcacggcgtgtaccccggctacctgatcacttcgtgcagtcc
gccctgatgatcgccggctcccgcgccatctaccgctggcagcaggccgtgccccccaagatgtccctggtgaagaacaccctg
gtgacttcaacttcgcctacaccctgctggtgctgaactactccgccgtgggatcatggtgctgtccatgcacgagaccctggcctc
ctacggctccgtgtactacgtgggcaccatcctgcccgtgaccctgatcctgctgggctacgtgatcaagcccggcaagtcccccc
gctccaaggcctccaaggagcagTGActcgag
CavigPLA2-1
SEQ ID NO: 122
ggtaccATGaacttcgacttcctgtccaacatcccctggttcggcgccaaggcctccgacaacgccggctcctccttcggctccg
ccaccatcgtgatccagcagcccccccccgtgtcccgcggatcgacatccgccactggggctggccctggtccgtgctgtccgtg
ctgccctggggcaagcccggctgcgacgagctgcgcgccccccccaccaccatcaaccgccgcctgaagcgcaacgccacct
ccatgcactcctccgccgtgcgcggcaacgccgaggccgcccgcgtgcgcaccgcccctacgtgtccaaggtgccctggcaca
ccggcaccgcggcctgctgtcccagctgacccccgctacggccactactgcggccccaactggtcctccggcaagaacggcgg
ctcccccgtgtgggaccagcgccccatcgactggctggactactgctgctactgccacgacatcggctacgacacccacgacca
ggccaagctgctggaggccgacctggccacctggagtgcctggagcgcccctcctaccccaccaagggcgacgcccacgtgg
cccacatgtacaagaccatgtgcgtgaccggcctgcgcaacgtgctgatcccctaccgcacccagctgctgcgcctgaactcccg
ccagcccctgatcgacttcggctggctgtccaacgccgcctggaagggctggaacgcccagaagtccTGActcgag
CignPLA2-1
SEQ ID NO: 123
ggtaccATGaacctggacttcctgtccaagatcccctggttcgaggccaaggcctccgagaaccccggcctgaacctgggctcc
accaccatcgtgatcaagcagccccgccagggatcgacatccgccactggggctggccctggtccgtgctgacctggggcaac
cgcgtgaccgacgaggtgcacgccccccccaccaccatcaaccgccgcctgaagcgcaacgccaccggccccgccgtgcag
ggcgacaccgaggccgcccgcctgcgcaccgcccctacgtgtccaaggtgccctggcacaccggcaccgcggcctgctgtccc
agctgacccccgctacggccactactgcggccccaactggtcctccggcaagaacggcggctcccccgtgtgggaccagcgcc
ccatcgactggctggactactgctgctactgccacgacatcggctacgacacccacgaccaggccaagctgctggaggccgacc
tggccacctggagtgcctggagcgcccctcctaccccaccaccggcgacgcccacgtggcccacatgtacaagaccatgtgcgt
gaccggcctgcgcaacgtgctgatcccctaccgcacccagctgctgcgcctgaacttccgccagcccctgatcgacttcggctggc
tgtccaacgccgcctggaagggctggtccgcccagaagaccTGActcgag
CuPSR23PLA2-2
SEQ ID NO: 124
ggtaccATGgtgcacctgccccacaccctgaagctgggcctggtgatcgccatctccatctccggcctgtgcttctcctccacccc
cgcccgcgccctgaacgtgggcatccaggccgccggcgtgaccgtgtccgtgggcaagggctgctcccgcaagtgcgagtccg
acactgcaaggtgccccccacctgcgctacggcaagtactgcggcctgatgtactccggctgccccggcgagaagccctgcgac
ggcctggacgcctgctgcatgaagcacgacgcctgcgtgcaggccaagaacaacgactacctgtcccaggagtgctcccagaa
cctgctgaactgcatggcctccaccgcatgtccggcggcaagcagacaagggctccacctgccaggtggacgaggtggtggac
gtgctgaccgtggtgatggaggccgccctgctggccggccgctacctgcacaagcccTGActcgag
CprocPLA2-2
SEQ ID NO: 125
ggtaccATGgtgcacctgccccacaccctgaagctgggcctggtgatcgccatctccatctccggcctgtgcctgtcctccacccc
cgcccgcgccctgaacgtgggcatccaggccgccggcgtgaccgtgtccgtgggcaagggctgctcccgcaagtgcgagtccg
acactgcaaggtgccccccacctgcgctacggcaagtactgcggcctgatgtactccggctgccccggcgagaagccctgcgac
ggcctggacgcctgctgcatgaagcacgacgcctgcgtgcaggccaagaacgacgactacctgtcccaggagtgctcccagaa
cctgctgaactgcatggcctccaccgcatgtccggcggcaagcagacaagggctccacctgccaggtggacgaggtggtggac
gtgctgaccgtggtgatggaggccgccctgctggccgccgctacctgcacaagcccTGActcgag
Nucleotide sequence of transforming DNA contained in pSZ5654
PmKASII
SEQ ID NO: 126
gtttaaacgccggtcaccacccgcatgctcgtactacagcgcacgcaccgcttcgtgatccaccgggtgaacgtagtcctcgacgg
aaacatctggttcgggcctcctgcttgcactcccgcccatgccgacaacctttctgctgttaccacgacccacaatgcaacgcgaca
cgaccgtgtgggactgatcggttcactgcacctgcatgcaattgtcacaagcgcttactccaattgtattcgtttgttttctgggagc
agttgctcgaccgcccgcgtcccgcaggcagcgatgacgtgtgcgtggcctgggtgtttcgtcgaaaggccagcaaccctaaatcg
caggcgatccggagattgggatctgatccgagtttggaccagatccgccccgatgcggcacgggaactgcatcgactcggcgcgg
aacccagctttcgtaaatgccagattggtgtccgatacctggatttgccatcagcgaaacaagacttcagcagcgagcgtatttgg
cgggcgtgctaccagggttgcatacattgcccatttctgtctggaccgctttactggcgcagagggtgagttgatggggttggcagg
catcgaaacgcgcgtgcatggtgtgcgtgtctgttttcggctgcacgaattcaatagtcggatgggcgacggtagaattgggtgtg
gcgctcgcgtgcatgcctcgccccgtcgggtgtcatgaccgggactggaatcccccctcgcgaccatcttgctaacgctcccgactc

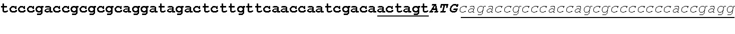

gccactgcttcggcgcccgcctgcccaccgcctcccgccgcgccgtgcgccgcgcctggtcccgcatcgcccggggcgcgccac
cgccgccgccgacgccaaccccgcccgccccgagcgccgcgtggtgatcaccggccagggcgtggtgacctccctgggccag
accatcgagcagttctactcctccctgctggagggcgtgtccggcatctcccagatccagaagttcgacaccaccggctacacc
accaccatcgccggcgagatcaagtccctgcagctggacccctacgtgcccaagcgctgggccaagcgcgtggacgacgtga
tcaagtacgtgtacatcgccggcaagcaggccctggagtccgccggcctgcccatcgaggccgccggcctggccggcgccgg
cctggaccccgccctgtgcggcgtgctgatcggcaccgccatggccggcatgacctccttcgccgccggcgtggaggccctgac
ccgcggcggcgtgcgcaagatgaaccccttctgcatccccttctccatctccaacatgggcggcgccatgctggccatggacatc
ggcttcatgggccccaactactccatctccaccgcctgcgccaccggcaactactgcatcctgggcgccgccgaccacatccgcc
gcggcgacgccaacgtgatgctggccggcggcgccgacgccgccatcatcccctccggcatcggcggcttcatcgcctgcaag
gccctgtccaagcgcaacgacgagcccgagcgcgcctcccgcccctgggacgccgaccgcgacggcttcgtgatgggcgagg
gcgccggcgtgctggtgctggaggagctggagcacgccaagcgccgcggcgccaccatcctggccgagctggtgggcggcg
ccgccacctccgacgcccaccacatgaccgagcccgacccccagggccgcggcgtgcgcctgtgcctggagcgcgccctggag
cgcgcccgcctggcccccgagcgcgtgggctacgtgaacgcccacggcacctccacccccgccggcgacgtggccgagtaccg
cgccatccgcgccgtgatcccccaggactccctgcgcatcaactccaccaagtccatgatcggccacctgctgggcggcgccgg
cgccgtggaggccgtggccgccatccaggccctgcgcaccggctggctgcaccccaacctgaacctggagaaccccgcccccg
gcgtggaccccgtggtgctggtgggcccccgcaaggagcgcgccgaggacctggacgtggtgctgtccaactccttcggcttc
ggcggccacaactcctgcgtgatcttccgcaagtacgacgagATGGACTACAAGGACCACGACGGCGACTACAA

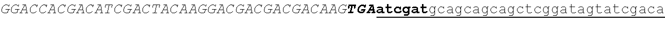

cactctggacgctggtcgtgtgatggactgttgccgccacacttgctgccttgacctgtgaatatccctgccgcttttatcaaacagcc
tcagtgtgtttgatcttgtgtgtacgcgcttttgcgagttgctagctgcttgtgctatttgcgaataccacccccagcatccccttccctc
gtttcatatcgcttgcatcccaaccgcaacttatctacgctgtcctgctatccctcagcgctgctcctgctcctgctcactgcccctcgca
cagccttggtttgggctccgcctgtattctcctggtactgcaacctgtaaaccagcactgcaatgctgatgcacgggaagtagtggga
tgggaacacaaatggagagctccgcgtctcgaacagagcgcgcagaggaacgctgaaggtctcgcctctgtcgcacctcagcgcg
gcatacaccacaataaccacctgacgaatgcgcttggttcttcgtccattagcgaagcgtccgttcacacacgtgccacgttggcg

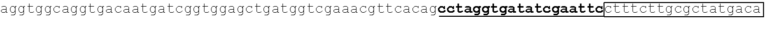

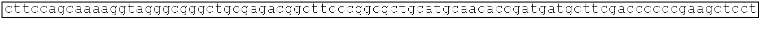

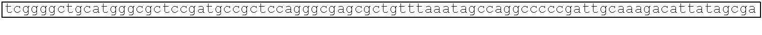

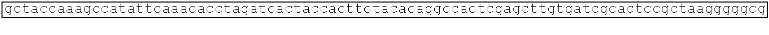

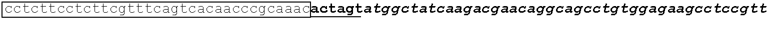

cacgatcgggacgctgcgcaaggccatccccgcgcactgtttcgagcgctcggcgcttcgtagcagcatgtacctggcctttg
acatcgcggtcatgtccctgctctacgtcgcgtcgacgtacatcgaccctgcaccggtgcctacgtgggtcaagtacggcatc
atgtggccgctctactggttcttccaggtgtgtttgagggttttggttgcccgtattgaggtcctggtggcgcgcatggaggag
aaggcgcctgtcccgctgacccccccggctaccctcccggcaccttccagggcgcgtacgagaagaaccagtagagcggcca
catgatgccgtacttgacccacgtaggcaccggtgcagggtcgatgtacgtcgacgcgacgtagagcagggacatgaccg
cgatgtcaaaggccaggtacatgctgctacgaagcgccgagcgctcgaaacagtgcgcggggatggccttgcgcagcgtc
ccgatcgtgaacggaggcttctccacaggctgcctgttcgtcttgatagccatctcgaggcagcagcagctcggatagtatcga
cacactctggacgctggtcgtgtgatggactgttgccgccacacttgctgccttgacctgtgaatatccctgccgcttttatcaaacag
cctcagtgtgtttgatcttgtgtgtacgcgcttttgcgagttgctagctgcttgtgctatttgcgaataccacccccagcatccccttccc
tcgtttcatatcgcttgcatcccaaccgcaacttatctacgctgtcctgctatccctcagcgctgctcctgctcctgctcactgcccctcg
cacagccttggtttgggctccgcctgtattctcctggtactgcaacctgtaaaccagcactgcaatgctgatgcacgggaagtagtgg
gatgggaacacaaatggaaagctgtagagctcgatctaagtaagattcgaagcgctcgaccgtgccggacggactgcagccccat
gtcgtagtgaccgccaatgtaagtgggctggcgtttccctgtacgtgagtcaacgtcactgcacgcgcaccaccctctcgaccggca

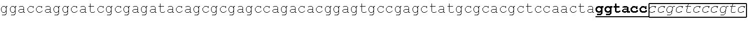

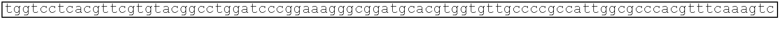

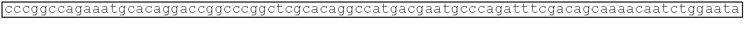

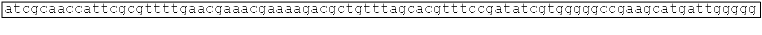

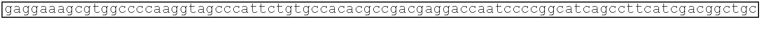

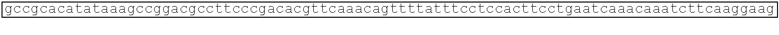

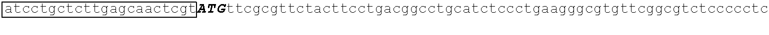

ctacaacggcctgggcctgacgccccagatgggctgggacaactggaacacgttcgcctgcgacgtctccgagcagctgctgc
tggacacggccgaccgcatctccgacctgggcctgaaggacatgggctacaagtacatcatcctggacgactgctggtcctcc
ggccgcgactccgacggcttcctggtcgccgacgagcagaagttccccaacggcatgggccacgtcgccgaccacctgcaca
acaactccttcctgttcggcatgtactcctccgcgggcgagtacacgtgcgccggctaccccggctccctgggccgcgaggagg
aggacgcccagttcttcgcgaacaaccgcgtggactacctgaagtacgacaactgctacaacaagggccagttcggcacgcc
cgagatctcctaccaccgctacaaggccatgtccgacgccctgaacaagacgggccgccccatcttctactccctgtgcaactg
gggccaggacctgaccttctactggggctccggcatcgcgaactcctggcgcatgtccggcgacgtcacggcggagttcacgc
gccccgactcccgctgcccctgcgacggcgacgagtacgactgcaagtacgccggcttccactgctccatcatgaacatcctga
acaaggccgcccccatgggccagaacgcgggcgtcggcggctggaacgacctggacaacctggaggtcggcgtcggcaac
ctgacggacgacgaggagaaggcgcacttctccatgtgggccatggtgaagtcccccctgatcatcggcgcgaacgtgaaca
acctgaaggcctcctcctactccatctactcccaggcgtccgtcatcgccatcaaccaggactccaacggcatccccgccacgcg
cgtctggcgctactacgtgtccgacacggacgagtacggccagggcgagatccagatgtggtccggccccctggacaacggc
gaccaggtcgtggcgctgctgaacggcggctccgtgtcccgccccatgaacacgaccctggaggagatcttcttcgactccaac
ctgggctccaagaagctgacctccacctgggacatctacgacctgtgggcgaaccgcgtcgacaactccacggcgtccgccat
cctgggccgcaacaagaccgccaccggcatcctgtacaacgccaccgagcagtcctacaaggacggcctgtccaagaacgac
acccgcctgttcggccagaagatcggctccctgtcccccaacgcgatcctgaacacgaccgtccccgcccacggcatcgcgttct

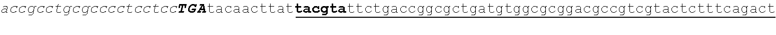

ttactcttgaggaattgaacctttctcgcttgctggcatgtaaacattggcgcaattaattgtgtgatgaagaaagggtggcacaaga
tggatcgcgaatgtacgagatcgacaacgatggtgattgttatgaggggccaaacctggctcaatcttgtcgcatgtccggcgcaat
gtgatccagcggcgtgactctcgcaacctggtagtgtgtgcgcaccgggtcgctttgattaaaactgatcgcattgccatcccgtcaa
ctcacaagcctactctagctcccattgcgcactcgggcgcccggctcgatcaatgttctgagcggagggcgaagcgtcaggaaatcg
tctcggcagctggaagcgcatggaatgcggagcggagatcgaatcaggatccttagggagcgacgagtgtgcgtgcggggctggc
gggagtgggacgccctcctcgctcctctctgttctgaacggaacaatcggccaccccgcgctacgcgccacgcatcgagcaacga
agaaaaccccccgatgataggttgcggtggctgccgggatatagatccggccgcacatcaaagggcccctccgccagagaagaa
gctcctttcccagcagactccttctgctgccaaaacacttctctgtccacagcaacaccaaaggatgaacagatcaacttgcgtctc
cgcgtagcttcctcggctagcgtgcttgcaacaggtccctgcactattatcttcctgctttcctctgaattatgcggcaggcgagcgct
cgctctggcgagcgctccttcgcgccgccctcgctgatcgagtgtacagtcaatgaatggtcctgggcgaagaacgagggaatttg
tgggtaaaacaagcatcgtctctcaggccccggcgcagtggccgttaaagtccaagaccgtgaccaggcagcgcagcgcgtccgt
gtgcgggccctgcctggcggctcggcgtgccaggctcgagagcagctccctcaggtcgccttggacggcctctgcgaggccggtga
gggcctgcaggagcgcctcgagcgtggcagtggcggtcgtatccgggtcgccggtcaccgcctgcgactcgccatccgaagagcg
tttaaac
Nucleotide sequence of transforming DNA contained in pSZ5868
GarmFATA1(G108A)
SEQ ID NO: 127
gaagagcgcccaatgtttaaacctcttttgctgcgtctcctcaggcttgggggcctccttgggcttgggtgccgccatgatctgcgcg
catcagagaaacgttgctggtaaaaaggagcgcccggctgcgcaatatatatataggcatgccaacacagcccaacctcactcg
ggagcccgtcccaccacccccaagtcgcgtgccttgacggcatactgctgcagaagcttcatgagaatgatgccgaacaagaggg
gcacgaggacccaatcccggacatccttgtcgataatgatctcgtgagtccccatcgtccgcccgacgctccggggagcccgccga
tgctcaagacgagagggccctcgaccaggaggggctggcccgggcgggcactggcgtcgaaggtgcgcccgtcgttcgcctgca
gtcctatgccacaaaacaagtcttctgacggggtgcgtttgctcccgtgcgggcaggcaacagaggtattcaccctggtcatgggg
agatcggcgatcgagctgggataagagatacggtcccgcgcaaggatcgctcatcctggtctgagccggacagtcattctggcaa
gcaatgacaacttgtcaggaccggaccgtgccatatatttctcacctagcgccgcaaaacctaacaatttgggagtcactgtgcca
ctgagttcgactggtagctgaatggagtcgctgctccactaaacgaattgtcagcaccgccagccggccgaggacccgagtcata

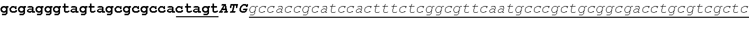

ggcgggctccgggccccggcgcccagcgaggcccctccccgtgcgcgggcgcgccatccccccccgcatcatcgtggtgtcctc
ctcctcctccaaggtgaaccccctgaagaccgaggccgtggtgtcctccggcctggccgaccgcctgcgcctgggctccctgacc
gaggacggcctgtcctacaaggagaagttcatcgtgcgctgctacgaggtgggcatcaacaagaccgccaccgtggagacc
atcgccaacctgctgcaggaggtgggctgcaaccacgcccagtccgtgggctactccaccgccggcttctccaccacccccacc
atgcgcaagctgcgcctgatctgggtgaccgcccgcatgcacatcgagatctacaagtaccccgcctggtccgacgtggtgga
gatcgagtcctggggccagggcgagggcaagatcggcacccgccgcgactggatcctgcgcgactacgccaccggccaggt
gatcggccgcgccacctccaagtgggtgatgatgaaccaggacacccgccgcctgcagaaggtggacgtggacgtgcgcga
cgagtacctggtgcactgcccccgcgagctgcgcctggccttccccgaggagaacaactcctccctgaagaagatctccaagct
ggaggacccctcccagtactccaagctgggcctggtgccccgccgcgccgacctggacatgaaccagcacgtgaacaacgtg
acctacatcggctgggtgctggagtccatgccccaggagatcatcgacacccacgagctgcagaccatcaccctggactaccg
ccgcgagtgccagcacgacgacgtggtggactccctgacctcccccgagccctccgaggacgccgaggccgtgttcaaccaca
acggcaccaacggctccgccaacgtgtccgccaacgaccacggctgccgcaacttcctgcacctgctgcgcctgtccggcaacg
gcctggagatcaaccgcggccgcaccgagtggcgcaagaagcccacccgcATGGACTACAAGGACCACGACGGCG
ACTACAAGGACCACGACATCGACTACAAGGACGACGACGACAAGTGAatcgatggagcgacgagtgtgcgt
gcggggctggcgggagtgggacgccctcctcgctcctctctgttctgaacggaacaatcggccaccccgcgctacgcgccacgcatc
gagcaacgaagaaaaccccccgatgataggttgcggtggctgccgggatatagatccggccgcacatcaaagggcccctccgcca
gagaagaagctcctttcccagcagactccttctgctgccaaaacacttctctgtccacagcaacaccaaaggatgaacagatcaact
tgcgtctccgcgtagcttcctcggctagcgtgcttgcaacaggtccctgcactattatcttcctgctttcctctgaattatgcggcaggc
gagcgctcgctctggcgagcgctccttcgcgccgccctcgctgatcgagtgtacagtcaatgaatggtgagctccgcgtctcgaaca
gagcgcgcagaggaacgctgaaggtctcgcctctgtcgcacctcagcgcggcatacaccacaataaccacctgacgaatgcgcttg
gttcttcgtccattagcgaagcgtccggttcacacacgtgccacgttggcgaggtggcaggtgacaatgatcggtggagctgatggt

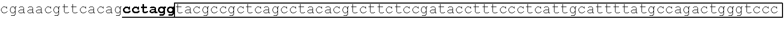

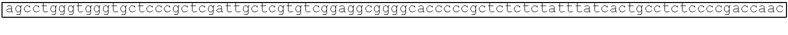

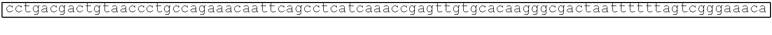

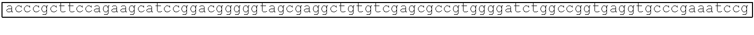

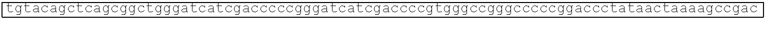

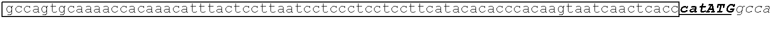

tcgccgccgccgccgtgatcgtgcccctgggcctgctgttcttcatctccggcctggtggtgaacctgatccaggccctgtgcttcg
tgctgatccgccccctgtccaagaacacctaccgcaagatcaaccgcgtggtggccgagctgctgtggctggagctgatctggc
tggtggactggtgggccggcgtgaagatcaaggtgttcatggaccccgagtccttcaacctgatgggcaaggagcacgccct
ggtggtggccaaccaccgctccgacatcgactggctggtgggctggctgctggcccagcgctccggctgcctgggctccgccct
ggccgtgatgaagaagtcctccaagttcctgcccgtgatcggctggtccatgtggttctccgagtacctgttcctggagcgctcct
gggccaaggacgagaacaccctgaaggccggcctgcagcgcctgaaggacttcccccgccccttctggctggccttcttcgtg
gagggcacccgcttcacccaggccaagttcctggccgcccaggagtacgccgcctcccagggcctgcccatcccccgcaacgt
gctgatcccccgcaccaagggcttcgtgtccgccgtgtcccacatgcgctccttcgtgcccgccatctacgacatgaccgtggcc
atccccaagtcctccccctcccccaccatgctgcgcctgttcaagggccagccctccgtggtgcacgtgcacatcaagcgctgcct
gatgaaggagctgcccgagaccgacgaggccgtggcccagtggtgcaaggacatgacgtggagaaggacaagctgctgg
acaagcacatcgccgaggacaccactccgaccagcccatgcaggacctgggccgccccatcaagtccctgctggtggtggcc
tcctgggcctgcctgatggcctacggcgccctgaagttcctgcagtgctcctccctgctgtcctcctggaagggcatcgccacttc
ctggtgggcctggccatcgtgaccatcctgatgcacatcctgatcctgttctcccagtccgagcgctccacccccgccaaggtgg

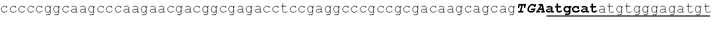

agggtggtcgactcgttggaggtgggtgtttttttttatcgagtgcgcggcgcggcaaacgggtccctttttatcgaggtgttcccaac
gccgcaccgccctcttaaaacaacccccaccaccacttgtcgaccttctcgtttgttatccgccacggcgccccggaggggcgtcgtc
tggccgcgcgggcagctgtatcgccgcgctcgctccaatggtgtgtaatcttggaaagataataatcgatggatgaggaggagagc
gtgggagatcagagcaaggaatatacagttggcacgaagcagcagcgtactaagctgtagcgtgttaagaaagaaaaactcgctg
ttaggctgttaatcaaggagcgtatcaataattaccgaccctatacctttatctccaacccaatcgcggcttaaggatctaagtaa
gattcgaagcgctcgaccgtgccggacggactgcagccccatgtcgtagtgaccgccaatgtaagtgggctggcgtttccctgtacg
tgagtcaacgtcactgcacgcgcaccaccctctcgaccggcaggaccaggcatcgcgagatacagcgcgagccagacacggagtg

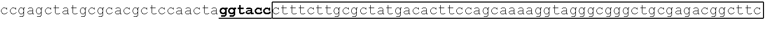

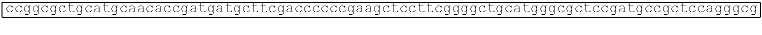

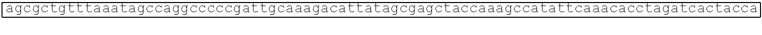

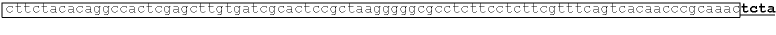

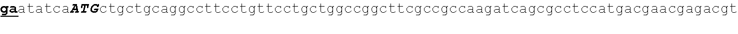

ccgaccgccccctggtgcacttcacccccaacaagggctggatgaacgaccccaacggcctgtggtacgacgagaaggacgc
caagtggcacctgtacttccagtacaacccgaacgacaccgtctgggggacgcccttgactggggccacgccacgtccgacg
acctgaccaactgggaggaccagcccatcgccatcgccccgaagcgcaacgactccggcgccttctccggctccatggtggtg
gactacaacaacacctccggcttcttcaacgacaccatcgacccgcgccagcgctgcgtggccatctggacctacaacaccccg
gagtccgaggagcagtacatctcctacagcctggacggcggctacaccttcaccgagtaccagaagaaccccgtgctggccg
ccaactccacccagttccgcgacccgaaggtcttctggtacgagccctcccagaagtggatcatgaccgcggccaagtcccag
gactacaagatcgagatctactcctccgacgacctgaagtcctggaagctggagtccgcgttcgccaacgagggcacctcgg
ctaccagtacgagtgccccggcctgatcgaggtccccaccgagcaggaccccagcaagtcctactgggtgatgttcatctccat
caaccccggcgccccggccggcggctccttcaaccagtacttcgtcggcagcttcaacggcacccacttcgaggccttcgacaa
ccagtcccgcgtggtggacttcggcaaggactactacgccctgcagaccttcttcaacaccgacccgacctacgggagcgccct
gggcatcgcgtgggcctccaactgggagtactccgccacgtgcccaccaacccctggcgctcctccatgtccctcgtgcgcaag
actccctcaacaccgagtaccaggccaacccggagacggagctgatcaacctgaaggccgagccgatcctgaacatcagca
acgccggcccctggagccggttcgccaccaacaccacgttgacgaaggccaacagctacaacgtcgacctgtccaacagcac
cggcaccctggagttcgagctggtgtacgccgtcaacaccacccagacgatctccaagtccgtgttcgcggacctctccctctgg
ttcaagggcctggaggaccccgaggagtacctccgcatgggcttcgaggtgtccgcgtcctccttcttcctggaccgcgggaac
agcaaggtgaagttcgtgaaggagaacccctacttcaccaaccgcatgagcgtgaacaaccagcccttcaagagcgagaac
gacctgtcctactacaaggtgtacggcttgctggaccagaacatcctggagctgtacttcaacgacggcgacgtcgtgtccacc
aacacctacttcatgaccaccgggaacgccctgggctccgtgaacatgacgacgggggtggacaacctgttctacatcgaca

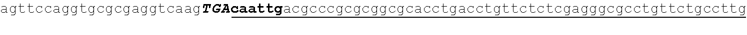

cgaaacaagcccctggagcatgcgtgcatgatcgtctctggcgccccgccgcgcggtttgtcgccctcgcgggcgccgcggccgcg
ggggcgcattgaaattgttgcaaaccccacctgacagattgagggcccaggcaggaaggcgttgagatggaggtacaggagtcaa
gtaactgaaagtttttatgataactaacaacaaagggtcgtttctggccagcgaatgacaagaacaagattccacatttccgtgtag
aggcttgccatcgaatgtgagcgggcgggccgcggacccgacaaaacccttacgacgtggtaagaaaaacgtggcgggcactgtc
cctgtagcctgaagaccagcaggagacgatcggaagcatcacagcacaggatcctgaggacagggtggttggctggatggggaa
acgctggtcgcgggattcgatcctgctgcttatatcctccctggaagcacacccacgactctgaagaagaaaacgtgcacacaca
caacccaaccggccgaatatttgcttccttatcccgggtccaagagagactgcgatgcccccctcaatcagcatcctcctccctgcc
gcttcaatcttccctgcttgcctgcgcccgcggtgcgccgtctgcccgcccagtcagtcactcctgcacaggccccttgtgcgcagtg
ctcctgtaccctttaccgctccttccattctgcgaggccccctattgaatgtattcgttgcctgtgtggccaagcgggctgctgggcgc
gccgccgtcgggcagtgctcggcgactttggcggaagccgattgttcttctgtaagccacgcgcttgctgctttgggaagagaagg
gggggggtactgaatggatgaggaggagaaggaggggtattggtattatctgagttggggaggcagggagagttggaaaatgt
aagtggcacgacgggcaaggagaatggtgagcatgtgcatggtgatgtcgttggtcgaggacgatcctgcacgcgtgtatctgat
gtagaatacggcaatcaccctagtctacatctataccttctccgtataacgccctttccaaatgccctcccgtttctctcctattcttg
atccacatgatgaccctggcactatttcaagggctggagaagagcgtttaaac
Nucleotide sequence of transforming DNA contained in pSZ6383
TcDGAT1 and GarmFATA1(G108A)
SEQ ID NO: 128
gctcttcgcgaaggtcattttccagaacaacgaccatggcttgtcttagcgatcgctcgaatgactgctagtgagtcgtacgctcga
cccagtcgctcgcaggagaacgcggcaactgccgagcttcggcttgccagtcgtgactcgtatgtgatcaggaatcattggcattg
gtagcattataattcggcttccgcgctgtttatgggcatggcaatgtctcatgcagtcgaccttagtcaaccaattctgggtggccag
ctccgggcgaccgggctccgtgtcgccgggcaccacctcctgccatgagtaacagggccgccctctcctcccgacgttggccaact
gaataccgtgtcttggggccctacatgatgggctgcctagtcgggcgggacgcgcaactgcccgcgcaatctgggacgtggtctga
atcctccaggcgggtttccccgagaaagaaagggtgccgatttcaaagcagagccatgtgccgggccctgtggcctgtgttggcgc
ctatgtagtcaccccccctcacccaattgtcgccagtttgcgcaatccataaactcaaaactgcagcttctgagctgcgctgttcaa

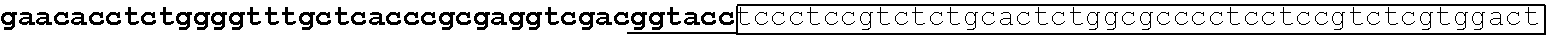

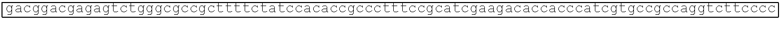

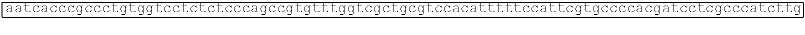

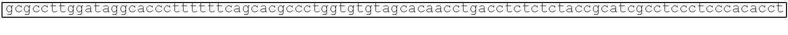

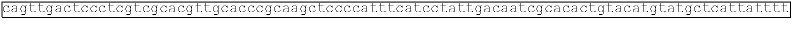

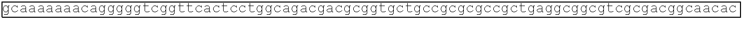

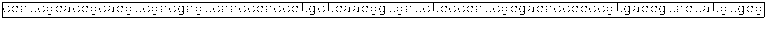

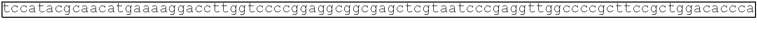

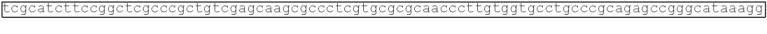

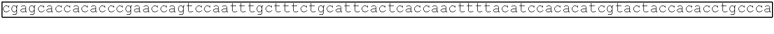

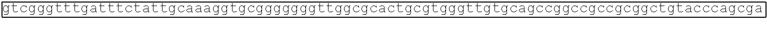

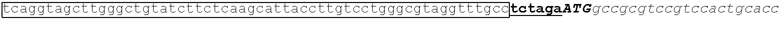

ctgatgtccgtggtctgcaacaacaagaaccactccgcccgccccaagctgcccaactcctccctgctgcccggcttcgacgtgg
tggtccaggccgcggccacccgcttcaagaaggagacgacgaccacccgcgccacgctgacgttcgacccccccacgaccaa
ctccgagcgcgccaagcagcgcaagcacaccatcgacccctcctcccccgacttccagcccatcccctccttcgaggagtgcttc
cccaagtccacgaaggagcacaaggaggtggtgcacgaggagtccggccacgtcctgaaggtgcccttccgccgcgtgcac
ctgtccggcggcgagcccgccttcgacaactacgacacgtccggcccccagaacgtcaacgcccacatcggcctggcgaagct
gcgcaaggagtggatcgaccgccgcgagaagctgggcacgccccgctacacgcagatgtactacgcgaagcagggcatcat
cacggaggagatgctgtactgcgcgacgcgcgagaagctggaccccgagttcgtccgctccgaggtcgcgcggggccgcgc
catcatcccctccaacaagaagcacctggagctggagcccatgatcgtgggccgcaagttcctggtgaaggtgaacgcgaac
atcggcaactccgccgtggcctcctccatcgaggaggaggtctacaaggtgcagtgggccaccatgtggggcgccgacacca
tcatggacctgtccacgggccgccacatccacgagacgcgcgagtggatcctgcgcaactccgcggtccccgtgggcaccgtc
cccatctaccaggcgctggagaaggtggacggcatcgcggagaacctgaactgggaggtgttccgcgagacgctgatcgag
caggccgagcagggcgtggactacttcacgatccacgcgggcgtgctgctgcgctacatccccctgaccgccaagcgcctgac
gggcatcgtgtcccgcggcggctccatccacgcgaagtggtgcctggcctaccacaaggagaacttcgcctacgagcactggg
acgacatcctggacatctgcaaccagtacgacgtcgccctgtccatcggcgacggcctgcgccccggctccatctacgacgcca
acgacacggcccagttcgccgagctgctgacccagggcgagctgacgcgccgcgcgtgggagaaggacgtgcaggtgatg
aacgagggccccggccacgtgcccatgcacaagatccccgagaacatgcagaagcagctggagtggtgcaacgaggcgcc
cttctacaccctgggccccctgacgaccgacatcgcgcccggctacgaccacatcacctccgccatcggcgcggccaacatcgg
cgccctgggcaccgccctgctgtgctacgtgacgcccaaggagcacctgggcctgcccaaccgcgacgacgtgaaggcgggc
gtcatcgcctacaagatcgccgcccacgcggccgacctggccaagcagcacccccacgcccaggcgtgggacgacgcgctgt
ccaaggcgcgcttcgagttccgctggatggaccagttcgcgctgtccctggaccccatgacggcgatgtccttccacgacgaga
cgctgcccgcggacggcgcgaaggtcgcccacttctgctccatgtgcggccccaagttctgctccatgaagatcacggaggac
atccgcaagtacgccgaggagaacggctacggctccgccgaggaggccatccgccagggcatggacgccatgtccgagga
gttcaacatcgccaagaagacgatctccggcgagcagcacggcgaggtcggcggcgagatctacctgcccgagtcctacgtc

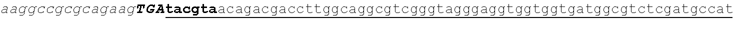

cgcacgcatccaacgaccgtatacgcatcgtccaatgaccgtcggtgtcctctctgcctccgttttgtgagatgtctcaggcttggtgc
atcctcgggtggccagccacgttgcgcgtcgtgctgcttgcctctcttgcgcctctgtggtactggaaaatatcatcgaggcccgttttt
ttgctcccatttcctttccgctacatcttgaaagcaaacgacaaacgaagcagcaagcaaagagcacgaggacggtgaacaagtct
gtcacctgtatacatctatttccccgcgggtgcacctactctctcctgccccggcagagtcagctgccttacgtgacggatcccgcg
tctcgaacagagcgcgcagaggaacgctgaaggtctcgcctctgtcgcacctcagcgcggcatacaccacaataaccacctgacga
atgcgcttggttcttcgtccattagcgaagcgtccggttcacacacgtgccacgttggcgaggtggcaggtgacaatgatcggtgga

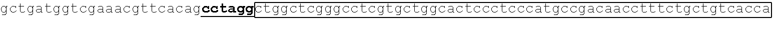

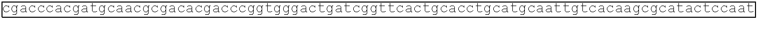

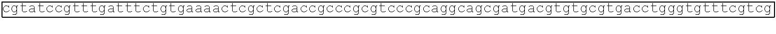

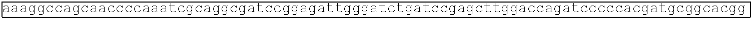

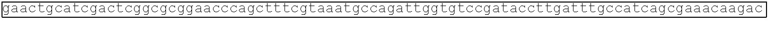

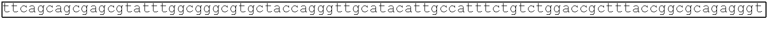

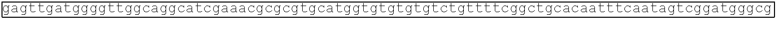

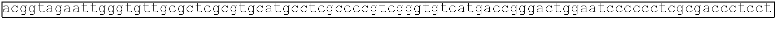

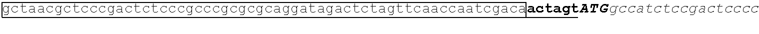

cgagatcctgggctccaccgccaccgtgacctcctcctcccactccgactccgacctgaacctgctgtccatccgccgccgcacct
ccaccaccgccgccgcccgcgcccccgaccgcgacgactccggcaacggcgaggccgtggacgaccgcgaccgcgtggagt
ccgccaacctgatgtccaacgtggccgagaacgccaacgagatgcccaactcctccgacacccgcttcacctaccgcccccgcg
tgcccgcccaccgccgcatcaaggagtcccccctgtcctccggcgccatcttcaagcagtcccacgccggcctgttcaacctgtgc
atcgtggtgctggtggccgtgaactcccgcctgatcatcgagaacctgatgaagtacggctggctgatccgctccggcttctggt
tctcctcccgctccctgtccgactggcccctgttcatgtgctgcctgaccctgcccatcttccccctggccgccttcgtggtggagaa
gctggtgcagcgcaactacatctccgagcccgtggtggtgttcctgcacgccatcatctccaccaccgccgtgctgtaccccgtg
atcgtgaacctgcgctgcgactccgccttcctgtccggcgtggccctgatgctgttcgcctgcatcgtgtggctgaagctggtgtc
ctacgcccacaccaacaacgacatgcgcgccctggccaagtccgccgagaagggcgacgtggacccctcctacgacgtgtcct
tcaagtccctggcctacttcatggtggcccccaccctgtgctaccagcagtcctacccccgcacccccgccgtgcgcaagtcctgg
gtggtgcgccagttcatcaagctgatcgtgttcaccggcctgatgggcttcatcatcgagcagtacatcaaccccatcgtgcag
aactcccagcaccccctgaagggcaacctgctgtacgccatcgagcgcgtgctgaagctgtccgtgcccaacctgtacgtgtgg
ctgtgcatgttctactgcttcttccacctgtggctgaacatcctggccgagctgctgcgcttcggcgaccgcgagttctacaagga
ctggtggaacgccaagaccgtggaggagtactggcgcatgtggaacatgcccgtgcacaagtggatggtgcgccacatctac
ttcccctgcctgcgcaacggcatccccaagggcgtggccatcgtgatcgccttcctggtgtccgccgtgttccacgagctgtgcat
cgccgtgccctgccacatgttcaagctgtgggccttcatcggcatcatgttccaggtgcccctggtgctgatcaccaactacctgc
aggacaagttccgctcctccatggtgggcaacatgatcttctggttcatcttctccatcctgggccagcccatgtgcgtgctgctgt

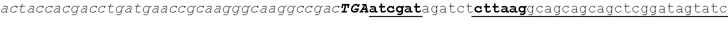

gacacactctggacgctggtcgtgtgatggactgttgccgccacacttgctgccttgacctgtgaatatccctgccgcttttatcaaac
agcctcagtgtgtttgatcttgtgtgtacgcgcttttgcgagttgctagctgcttgtgctatttgcgaataccacccccagcatccccttc
cctcgtttcatatcgcttgcatcccaaccgcaacttatctacgctgtcctgctatccctcagcgctgctcctgctcctgctcactgcccct
cgcacagccttggtttgggctccgcctgtattctcctggtactgcaacctgtaaaccagcactgcaatgctgatgcacgggaagtagt
gggatgggaacacaaatggacttaaggatctaagtaagattcgaagcgctcgaccgtgccggacggactgcagccccatgtcgta
gtgaccgccaatgtaagtgggctggcgtttccctgtacgtgagtcaacgtcactgcacgcgcaccaccctctcgaccggcaggacca
ggcatcgcgagatacagcgcgagccagacacggagtgccgagctatgcgcacgctccaactagatatcatgtggatgatgagcat

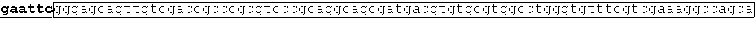

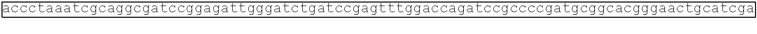

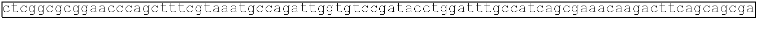

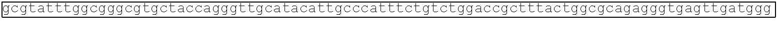

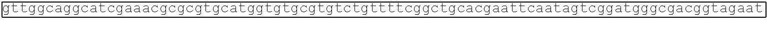

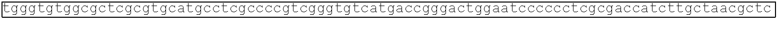

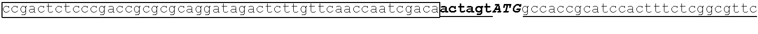

aatgcccgctgcggcgacctgcgtcgctcggcgggctccagggccccggcgcccagcgaggcccctccccgtgcgcggcgcgc
catccccccccgcatcatcgtggtgtcctcctcctcctccaaggtgaaccccctgaagaccgaggccgtggtgtcctccggcctgg
ccgaccgcctgcgcctgggctccctgaccgaggacggcctgtcctacaaggagaagttcatcgtgcgctgctacgaggtgggc
atcaacaagaccgccaccgtggagaccatcgccaacctgctgcaggaggtgggctgcaaccacgcccagtccgtgggctact
ccaccgccggcttctccaccacccccaccatgcgcaagctgcgcctgatctgggtgaccgcccgcatgcacatcgagatctaca
agtaccccgcctggtccgacgtggtggagatcgagtcctggggccagggcgagggcaagatcggcacccgccgcgactgga
tcctgcgcgactacgccaccggccaggtgatcggccgcgccacctccaagtgggtgatgatgaaccaggacacccgccgcctg
cagaaggtggacgtggacgtgcgcgacgagtacctggtgcactgcccccgcgagctgcgcctggccttccccgaggagaaca
actcctccctgaagaagatctccaagctggaggacccctcccagtactccaagctgggcctggtgccccgccgcgccgacctgg
acatgaaccagcacgtgaacaacgtgacctacatcggctgggtgctggagtccatgccccaggagatcatcgacacccacga
gctgcagaccatcaccctggactaccgccgcgagtgccagcacgacgacgtggtggactccctgacctcccccgagccctccg
aggacgccgaggccgtgttcaaccacaacggcaccaacggctccgccaacgtgtccgccaacgaccacggctgccgcaactt
cctgcacctgctgcgcctgtccggcaacggcctggagatcaaccgcggccgcaccgagtggcgcaagaagcccacccgcAT
GGACTACAAGGACCACGACGGCGACTACAAGGACCACGACATCGACTACAAGGACGACGACGACAA

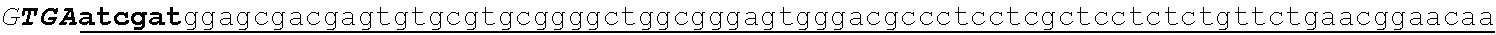

tcggccaccccgcgctacgcgccacgcatcgagcaacgaagaaaaccccccgatgataggttgcggtggctgccgggatatagat
ccggccgcacatcaaagggcccctccgccagagaagaagctcctttcccagcagactccttctgctgccaaaacacttctctgtcca
cagcaacaccaaaggatgaacagatcaacttgcgtctccgcgtagcttcctcggctagcgtgcttgcaacaggtccctgcactattat
cttcctgctttcctctgaattatgcggcaggcgagcgctcgctctggcgagcgctccttcgcgccgccctcgctgatcgagtgtacagt
caatgaatggtgagctcctcactcagcgcgcctgcgcggggatgcggaacgccgccgccgccttgtcttttgcacgcgcgactccgt
cgcttcgcgggtggcacccccattgaaaaaaacctcaattctgtttgtggaagacacggtgtacccccaaccacccacctgcacct
ctattattggtattattgacgcgggagcgggcgttgtactctacaacgtagcgtctctggttttcagctggctcccaccattgtaaatt
cttgctaaaatagtgcgtggttatgtgagaggtatggtgtaacagggcgtcagtcatgttggttttcgtgctgatctcgggcacaag
gcgtcgtcgacgtgacgtgcccgtgatgagagcaataccgcgctcaaagccgacgcatggcctttactccgcactccaaacgact
gtcgctcgtatttttcggatatctattttttaagagcgagcacagcgccgggcatgggcctgaaaggcctcgcggccgtgctcgtgg
tgggggccgcgagcgcgtggggcatcgcggcagtgcaccaggcgcagacggaggaacgcatggtgagtgcgcatcacaagatg
catgtcttgttgtctgtactataatgctagagcatcaccaggggcttagtcatcgcacctgctttggtcattacagaaattgcacaag
ggcgtcctccgggatgaggagatgtaccagctcaagctggagcggcttcgagccaagcaggagcgcggcgcatgacgacctacc
cacatgcgaagagc
Nucleotide sequence of transforming DNA contained in pSZ6384
TcDGAT2-and GarmFATA1(G108A)
SEQ ID NO: 129
gctcttcgcgaaggtcattttccagaacaacgaccatggcttgtcttagcgatcgctcgaatgactgctagtgagtcgtacgctcga
cccagtcgctcgcaggagaacgcggcaactgccgagcttcggcttgccagtcgtgactcgtatgtgatcaggaatcattggcattg
gtagcattataattcggcttccgcgctgtttatgggcatggcaatgtctcatgcagtcgaccttagtcaaccaattctgggtggccag
ctccgggcgaccgggctccgtgtcgccgggcaccacctcctgccatgagtaacagggccgccctctcctcccgacgttggccaact
gaataccgtgtcttggggccctacatgatgggctgcctagtcgggcgggacgcgcaactgcccgcgcaatctgggacgtggtctga
atcctccaggcgggtttccccgagaaagaaagggtgccgatttcaaagcagagccatgtgccgggccctgtggcctgtgttggcgc
ctatgtagtcaccccccctcacccaattgtcgccagtttgcgcaatccataaactcaaaactgcagcttctgagctgcgctgttcaa

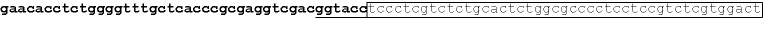

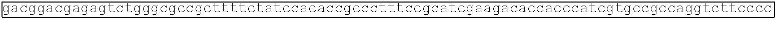

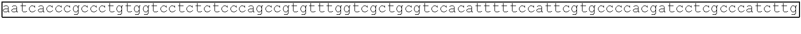

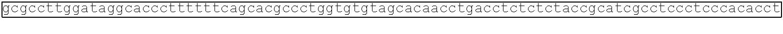

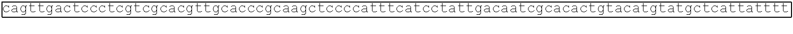

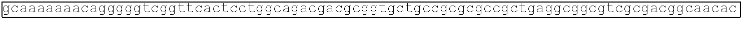

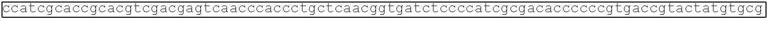

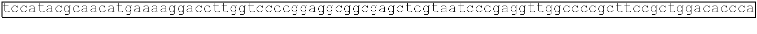

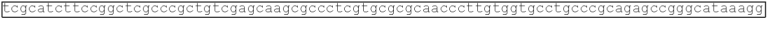

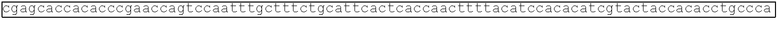

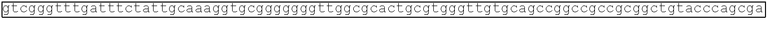

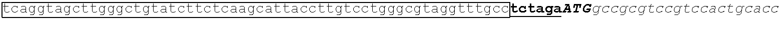

ctgatgtccgtggtctgcaacaacaagaaccactccgcccgccccaagctgcccaactcctccctgctgcccggcttcgacgtgg
tggtccaggccgcggccacccgcttcaagaaggagacgacgaccacccgcgccacgctgacgttcgacccccccacgaccaa
ctccgagcgcgccaagcagcgcaagcacaccatcgacccctcctcccccgacttccagcccatcccctccttcgaggagtgcttc
cccaagtccacgaaggagcacaaggaggtggtgcacgaggagtccggccacgtcctgaaggtgcccttccgccgcgtgcac
ctgtccggcggcgagcccgccttcgacaactacgacacgtccggcccccagaacgtcaacgcccacatcggcctggcgaagct
gcgcaaggagtggatcgaccgccgcgagaagctgggcacgccccgctacacgcagatgtactacgcgaagcagggcatcat
cacggaggagatgctgtactgcgcgacgcgcgagaagctggaccccgagttcgtccgctccgaggtcgcgcggggccgcgc
catcatcccctccaacaagaagcacctggagctggagcccatgatcgtgggccgcaagttcctggtgaaggtgaacgcgaac
atcggcaactccgccgtggcctcctccatcgaggaggaggtctacaaggtgcagtgggccaccatgtggggcgccgacacca
tcatggacctgtccacgggccgccacatccacgagacgcgcgagtggatcctgcgcaactccgcggtccccgtgggcaccgtc
cccatctaccaggcgctggagaaggtggacggcatcgcggagaacctgaactgggaggtgttccgcgagacgctgatcgag
caggccgagcagggcgtggactacttcacgatccacgcgggcgtgctgctgcgctacatccccctgaccgccaagcgcctgac
gggcatcgtgtcccgcggcggctccatccacgcgaagtggtgcctggcctaccacaaggagaacttcgcctacgagcactggg
acgacatcctggacatctgcaaccagtacgacgtcgccctgtccatcggcgacggcctgcgccccggctccatctacgacgcca
acgacacggcccagttcgccgagctgctgacccagggcgagctgacgcgccgcgcgtgggagaaggacgtgcaggtgatg
aacgagggccccggccacgtgcccatgcacaagatccccgagaacatgcagaagcagctggagtggtgcaacgaggcgcc
cttctacaccctgggccccctgacgaccgacatcgcgcccggctacgaccacatcacctccgccatcggcgcggccaacatcgg
cgccctgggcaccgccctgctgtgctacgtgacgcccaaggagcacctgggcctgcccaaccgcgacgacgtgaaggcgggc
gtcatcgcctacaagatcgccgcccacgcggccgacctggccaagcagcacccccacgcccaggcgtgggacgacgcgctgt
ccaaggcgcgcttcgagttccgctggatggaccagttcgcgctgtccctggaccccatgacggcgatgtccttccacgacgaga
cgctgcccgcggacggcgcgaaggtcgcccacttctgctccatgtgcggccccaagttctgctccatgaagatcacggaggac
atccgcaagtacgccgaggagaacggctacggctccgccgaggaggccatccgccagggcatggacgccatgtccgagga
gttcaacatcgccaagaagacgatctccggcgagcagcacggcgaggtcggcggcgagatctacctgcccgagtcctacgtc

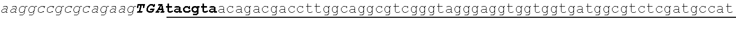

cgcacgcatccaacgaccgtatacgcatcgtccaatgaccgtcggtgtcctctctgcctccgttttgtgagatgtctcaggcttggtgc
atcctcgggtggccagccacgttgcgcgtcgtgctgcttgcctctcttgcgcctctgtggtactggaaaatatcatcgaggcccgttttt
ttgctcccatttcctttccgctacatcttgaaagcaaacgacaaacgaagcagcaagcaaagagcacgaggacggtgaacaagtct
gtcacctgtatacatctatttccccgcgggtgcacctactctctctcctgccccggcagagtcagctgccttacgtgacggatcccgcg
tctcgaacagagcgcgcagaggaacgctgaaggtctcgcctctgtcgcacctcagcgcggcatacaccacaataaccacctgacga
atgcgcttggttcttcgtccattagcgaagcgtccggttcacacacgtgccacgttggcgaggtggcaggtgacaatgatcggtgga

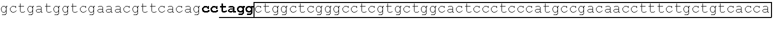

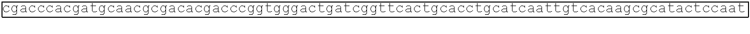

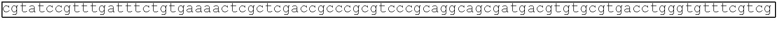

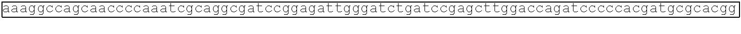

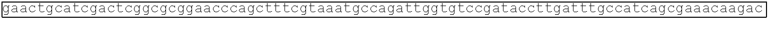

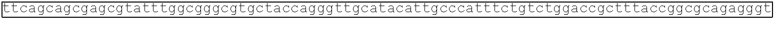

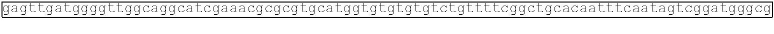

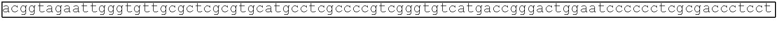

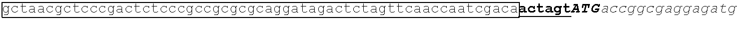

gaggagcgcaaggccaccggctaccgcgagttctccggccgccacgagttcccctccaacaccatgcacgccctgctggccat
gggcatctggctgggcgccatccacttcaacgccctgctgctgctgttctccttcctgttcctgcccttctccaagttcctggtggtgt
tcggcctgctgctgctgttcatgatcctgcccatcgacccctactccaagttcggccgccgcctgtcccgctacatctccaagcacg
cctgctcctacttccccatcaccctgcacgtggaggacatccacgccttccaccccgaccgcgcctacgtgttcggcttcgagccc
cactccgtgctgcccatcggcgtggtggccctggccgacctgaccggcttcatgcccctgcccaagatcaaggtgctggcctcct
ccgccgtgttctacacccccttcctgcgccacatctggacctggctgggcctgacccccgccaccaagaagaacttctcctccctg
ctggacgccggctactcctgcatcctggtgcccggcggcgtgcaggagaccttccacatggagcccggctccgagatcgccttc
ctgcgcgcccgccgcggcttcgtgcgcatcgccatggagatgggctcccccctggtgcccgtgttctgcttcggccagtcccacgt
gtacaagtggtggaagcccggcggcaagttctacctgcagttctcccgcgccatcaagttcacccccatcttcttctggggcatct
tcggctcccccctgccctaccagcaccccatgcacgtggtggtgggcaagcccatcgacgtgaagaagaacccccagcccatc
gtggaggaggtgatcgaggtgcacgaccgcttcgtggaggccctgcaggacctgttcgagcgccacaaggcccaggtgggc

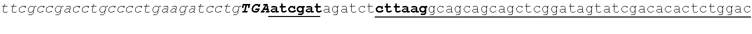

gctggtcgtgtgatggactgttgccgccacacttgctgccttgacctgtgaatatccctgccgcttttatcaaacagcctcagtgtgttt
gatcttgtgtgtacgcgcttttgcgagttgctagctgcttgtgctatttgcgaataccacccccagcatccccttccctcgtttcatatcg
cttgcatcccaaccgcaacttatctacgctgtcctgctatccctcagcgctgctcctgctcctgctcactgcccctcgcacagccttggt
ttgggctccgcctgtattctcctggtactgcaacctgtaaaccagcactgcaatgctgatgcacgggaagtagtgggatgggaacac
aaatggacttaaggatctaagtaagattcgaagcgctcgaccgtgccggacggactgcagccccatgtcgtagtgaccgccaatgt
aagtgggctggcgtttccctgtacgtgagtcaacgtcactgcacgcgcaccaccctctcgaccggcaggaccaggcatcgcgagat

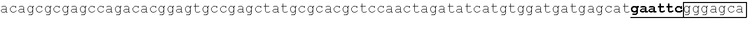

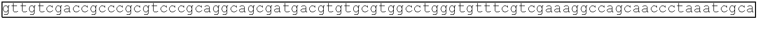

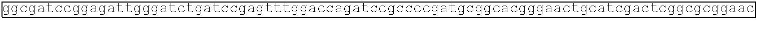

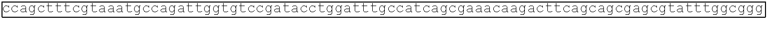

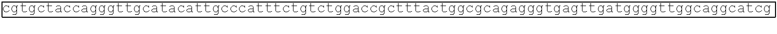

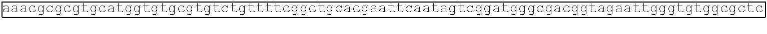

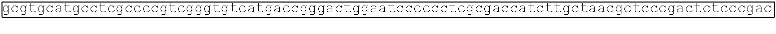

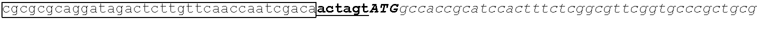

gcgacctgcgtcgctcggcgggctccgggccccggcgcccagcgaggcccctccccgtgcgcgggcgcgccatccccccccgca
tcatcgtggtgtcctcctcctcctccaaggtgaaccccctgaagaccgaggccgtggtgtcctccggcctggccgaccgcctgcg
cctgggctccctgaccgaggacggcctgtcctacaaggagaagttcatcgtgcgctgctacgaggtgggcatcaacaagacc
gccaccgtggagaccatcgccaacctgctgcaggaggtgggctgcaaccacgcccagtccgtgggctactccaccgccggctt
ctccaccacccccaccatgcgcaagctgcgcctgatctgggtgaccgcccgcatgcacatcgagatctacaagtaccccgcctg
gtccgacgtggtggagatcgagtcctggggccagggcgagggcaagatcggcacccgccgcgactggatcctgcgcgacta
cgccaccggccaggtgatcggccgcgccacctccaagtgggtgatgatgaaccaggacacccgccgcctgcagaaggtgga
cgtggacgtgcgcgacgagtacctggtgcactgcccccgcgagctgcgcctggccttccccgaggagaacaactcctccctga
agaagatctccaagctggaggacccctcccagtactccaagctgggcctggtgccccgccgcgccgacctggacatgaacca
gcacgtgaacaacgtgacctacatcggctgggtgctggagtccatgccccaggagatcatcgacacccacgagctgcagacc
atcaccctggactaccgccgcgagtgccagcacgacgacgtggtggactccctgacctcccccgagccctccgaggacgccga
ggccgtgttcaaccacaacggcaccaacggctccgccaacgtgtccgccaacgaccacggctgccgcaacttcctgcacctgct
gcgcctgtccggcaacggcctggagatcaaccgcggccgcaccgagtggcgcaagaagcccacccgcATGGACTACAA

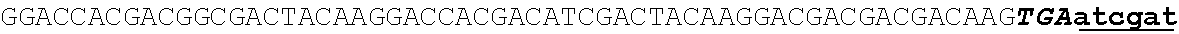

ggagcgacgagtgtgcgtgcggggctggcgggagtgggacgccctcctcgctcctctctgttctgaacggaacaatcggccacccc
gcgctacgcgccacgcatcgagcaacgaagaaaaccccccgatgataggttgcggtggctgccgggatatagatccggccgcaca
tcaaagggcccctccgccagagaagaagctcctttcccagcagactccttctgctgccaaaacacttctctgtccacagcaacacca
aaggatgaacagatcaacttgcgtctccgcgtagcttcctcggctagcgtgcttgcaacaggtccctgcactattatcttcctgctttc
ctctgaattatgcggcaggcgagcgctcgctctggcgagcgctccttcgcgccgccctcgctgatcgagtgtacagtcaatgaatggt
gagctcctcactcagcgcgcctgcgcggggatgcggaacgccgccgccgccttgtcttttgcacgcgcgactccgtcgcttcgcggg
tggcacccccattgaaaaaaacctcaattctgtttgtggaagacacggtgtacccccaaccacccacctgcacctctattattggta
ttattgacgcgggagcgggcgttgtactctacaacgtagcgtctctggttttcagctggctcccaccattgtaaattcttgctaaaat
agtgcgtggttatgtgagaggtatggtgtaacagggcgtcagtcatgttggttttcgtgctgatctcgggcacaaggcgtcgtcgac
gtgacgtgcccgtgatgagagcaataccgcgctcaaagccgacgcatggcctttactccgcactccaaacgactgtcgctcgtatt
tttcggatatctattttttaagagcgagcacagcgccgggcatgggcctgaaaggcctcgcggccgtgctcgtggtgggggccgcg
agcgcgtggggcatcgcggcagtgcaccaggcgcagacggaggaacgcatggtgagtgcgcatcacaagatgcatgtcttgttg
tctgtactataatgctagagcatcaccaggggcttagtcatcgcacctgctttggtcattacagaaattgcacaagggcgtcctccg
ggatgaggagatgtaccagctcaagctggagcggcttcgagccaagcaggagcgcggcgcatgacgacctacccacatgcgaa
gagc
Nucleotide sequence of transforming DNA contained in pSZ6377
GarmFATA1(G108A)
SEQ ID NO: 130
gctcttcgcgaaggtcattttccagaacaacgaccatggcttgtcttagcgatcgctcgaatgactgctagtgagtcgtacgctcga
cccagtcgctcgcaggagaacgcggcaactgccgagcttcggcttgccagtcgtgactcgtatgtgatcaggaatcattggcattg
gtagcattataattcggcttccgcgctgtttatgggcatggcaatgtctcatgcagtcgaccttagtcaaccaattctgggtggccag
ctccgggcgaccgggctccgtgtcgccgggcaccacctcctgccatgagtaacagggccgccctctcctcccgacgttggccaact
gaataccgtgtcttggggccctacatgatgggctgcctagtcgggcgggacgcgcaactgcccgcgcaatctgggacgtggtctga
atcctccaggcgggtttccccgagaaagaaagggtgccgatttcaaagcagagccatgtgccgggccctgtggcctgtgttggcgc
ctatgtagtcaccccccctcacccaattgtcgccagtttgcgcaatccataaactcaaaactgcagcttctgagctgcgctgttcaa

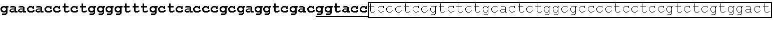

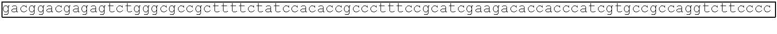

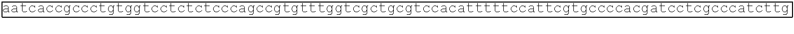

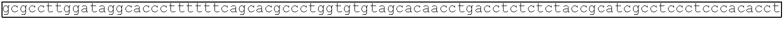

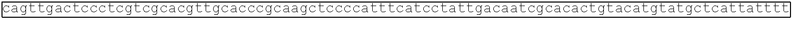

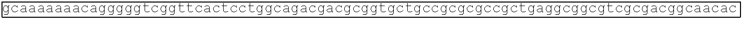

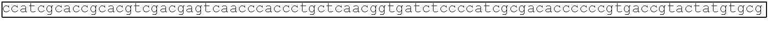

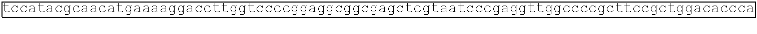

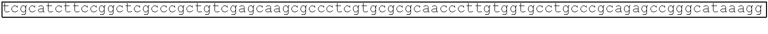

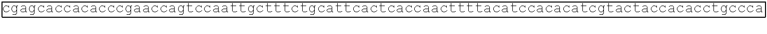

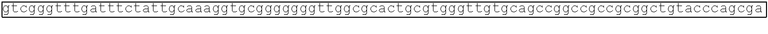

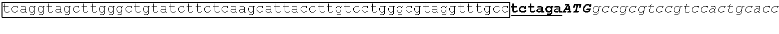

ctgatgtccgtggtctgcaacaacaagaaccactccgcccgccccaagctgcccaactcctccctgctgcccggcttcgacgtgg
tggtccaggccgcggccacccgcttcaagaaggagacgacgaccacccgcgccacgctgacgttcgacccccccacgaccaa
ctccgagcgcgccaagcagcgcaagcacaccatcgacccctcctcccccgacttccagcccatcccctccttcgaggagtgcttc
cccaagtccacgaaggagcacaaggaggtggtgcacgaggagtccggccacgtcctgaaggtgcccttccgccgcgtgcac
ctgtccggcggcgagcccgccttcgacaactacgacacgtccggcccccagaacgtcaacgcccacatcggcctggcgaagct
gcgcaaggagtggatcgaccgccgcgagaagctgggcacgccccgctacacgcagatgtactacgcgaagcagggcatcat
cacggaggagatgctgtactgcgcgacgcgcgagaagctggaccccgagttcgtccgctccgaggtcgcgcggggccgcgc
catcatcccctccaacaagaagcacctggagctggagcccatgatcgtgggccgcaagttcctggtgaaggtgaacgcgaac
atcggcaactccgccgtggcctcctccatcgaggaggaggtctacaaggtgcagtgggccaccatgtggggcgccgacacca
tcatggacctgtccacgggccgccacatccacgagacgcgcgagtggatcctgcgcaactccgcggtccccgtgggcaccgtc
cccatctaccaggcgctggagaaggtggacggcatcgcggagaacctgaactgggaggtgttccgcgagacgctgatcgag
caggccgagcagggcgtggactacttcacgatccacgcgggcgtgctgctgcgctacatccccctgaccgccaagcgcctgac
gggcatcgtgtcccgcggcggctccatccacgcgaagtggtgcctggcctaccacaaggagaacttcgcctacgagcactggg
acgacatcctggacatctgcaaccagtacgacgtcgccctgtccatcggcgacggcctgcgccccggctccatctacgacgcca
acgacacggcccagttcgccgagctgctgacccagggcgagctgacgcgccgcgcgtgggagaaggacgtgcaggtgatg
aacgagggccccggccacgtgcccatgcacaagatccccgagaacatgcagaagcagctggagtggtgcaacgaggcgcc
cttctacaccctgggccccctgacgaccgacatcgcgcccggctacgaccacatcacctccgccatcggcgcggccaacatcgg
cgccctgggcaccgccctgctgtgctacgtgacgcccaaggagcacctgggcctgcccaaccgcgacgacgtgaaggcgggc
gtcatcgcctacaagatcgccgcccacgcggccgacctggccaagcagcacccccacgcccaggcgtgggacgacgcgctgt
ccaaggcgcgcttcgagttccgctggatggaccagttcgcgctgtccctggaccccatgacggcgatgtccttccacgacgaga
cgctgcccgcggacggcgcgaaggtcgcccacttctgctccatgtgcggccccaagttctgctccatgaagatcacggaggac
atccgcaagtacgccgaggagaacggctacggctccgccgaggaggccatccgccagggcatggacgccatgtccgagga
gttcaacatcgccaagaagacgatctccggcgagcagcacggcgaggtcggcggcgagatctacctgcccgagtcctacgtc

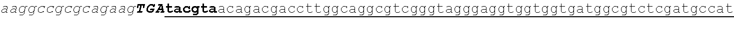

cgcacgcatccaacgaccgtatacgcatcgtccaatgaccgtcggtgtcctctctgcctccgttttgtgagatgtctcaggcttggtgc
atcctcgggtggccagccacgttgcgcgtcgtgctgcttgcctctcttgcgcctctgtggtactggaaaatatcatcgaggcccgttttt
ttgctcccatttcctttccgctacatcttgaaagcaaacgacaaacgaagcagcaagcaaagagcacgaggacggtgaacaagtct
gtcacctgtatacatctatttccccgcgggtgcacctactctctctcctgccccggcagagtcagctgccttacgtgacggatcccgcg
tctcgaacagagcgcgcagaggaacgctgaaggtctcgcctctgtcgcacctcagcgcggcatacaccacaataaccacctgacga
atgcgcttggttcttcgtccattagcgaagcgtccggttcacacacgtgccacgttggcgaggtggcaggtgacaatgatcggtgga

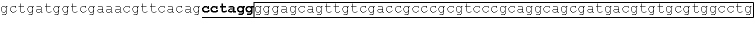

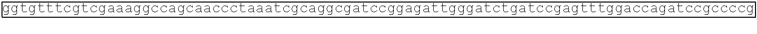

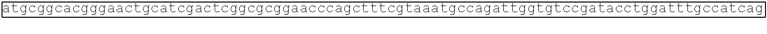

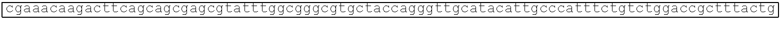

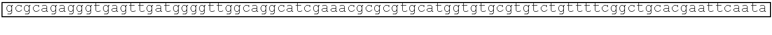

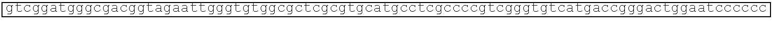

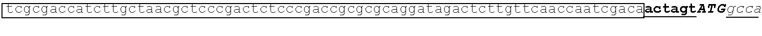

ccgcatccactttctcggcgttcaatgcccgctgcggcgacctgcgtcgctcggcgggctccgggccccggcgcccagcgaggc
ccctccccgtgcgcgggcgcgccatccccccccgcatcatcgtggtgtcctcctcctcctccaaggtgaaccccctgaagaccgag
gccgtggtgtcctccggcctggccgaccgcctgcgcctgggctccctgaccgaggacggcctgtcctacaaggagaagttcatc
gtgcgctgctacgaggtgggcatcaacaagaccgccaccgtggagaccatcgccaacctgctgcaggaggtgggctgcaac
cacgcccagtccgtgggctactccaccgccggcttctccaccacccccaccatgcgcaagctgcgcctgatctgggtgaccgccc
gcatgcacatcgagatctacaagtaccccgcctggtccgacgtggtggagatcgagtcctggggccagggcgagggcaaga
tcggcacccgccgcgactggatcctgcgcgactacgccaccggccaggtgatcggccgcgccacctccaagtgggtgatgatg
aaccaggacacccgccgcctgcagaaggtggacgtggacgtgcgcgacgagtacctggtgcactgcccccgcgagctgcgcc
tggccttccccgaggagaacaactcctccctgaagaagatctccaagctggaggacccctcccagtactccaagctgggcctg
gtgccccgccgcgccgacctggacatgaaccagcacgtgaacaacgtgacctacatcggctgggtgctggagtccatgcccca
ggagatcatcgacacccacgagctgcagaccatcaccctggactaccgccgcgagtgccagcacgacgacgtggtggactcc
ctgacctcccccgagccctccgaggacgccgaggccgtgacaaccacaacggcaccaacggctccgccaacgtgtccgccaa
cgaccacggctgccgcaacttcctgcacctgctgcgcctgtccggcaacggcctggagatcaaccgcggccgcaccgagtggc
gcaagaagcccacccgcATGGACTACAAGGACCACGACGGCGACTACAAGGACCACGACATCGACTACA

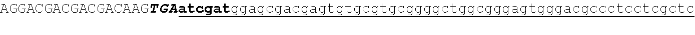

ctctctgttctgaacggaacaatcggccaccccgcgctacgcgccacgcatcgagcaacgaagaaaaccccccgatgataggttgc
ggtggctgccgggatatagatccggccgcacatcaaagggcccctccgccagagaagaagctcctttcccagcagactccttctgct
gccaaaacacttctctgtccacagcaacaccaaaggatgaacagatcaacttgcgtctccgcgtagcttcctcggctagcgtgcttg
caacaggtccctgcactattatcttcctgctttcctctgaattatgcggcaggcgagcgctcgctctggcgagcgctccttcgcgccgc
cctcgctgatcgagtgtacagtcaatgaatggtgagctcctcactcagcgcgcctgcgcggggatgcggaacgccgccgccgcctt
gtcttttgcacgcgcgactccgtcgcttcgcgggtggcacccccattgaaaaaaacctcaattctgtttgtggaagacacggtgtac
ccccaaccacccacctgcacctctattattggtattattgacgcgggagcgggcgttgtactctacaacgtagcgtctctggttttca
gctggctcccaccattgtaaattcttgctaaaatagtgcgtggttatgtgagaggtatggtgtaacagggcgtcagtcatgttggtt
ttcgtgctgatctcgggcacaaggcgtcgtcgacgtgacgtgcccgtgatgagagcaataccgcgctcaaagccgacgcatggcc
tttactccgcactccaaacgactgtcgctcgtatttttcggatatctattttttaagagcgagcacagcgccgggcatgggcctgaa
aggcctcgcggccgtgctcgtggtgggggccgcgagcgcgtggggcatcgcggcagtgcaccaggcgcagacggaggaacgcat
ggtgagtgcgcatcacaagatgcatgtcttgttgtctgtactataatgctagagcatcaccaggggcttagtcatcgcacctgcttt
ggtcattacagaaattgcacaagggcgtcctccgggatgaggagatgtaccagctcaagctggagcggcttcgagccaagcagg
agcgcggcgcatgacgacctacccacatgcgaagagc
Nucleotide sequence of transforming DNA contained in pSZ6315 BnOTE
SEQ ID NO: 131
caccggcgcgctgcttcgcgtgccgggtgcagcaatcagatccaagtctgacgacttgcgcgcacgcgccggatccttcaattccaaagtgtcg
tccgcgtgcgcttcttcgccttcgtcctcttgaacatccagcgacgcaagcgcagggcgctgggcggctggcgtcccgaaccggcctcggcgcac
gcggctgaaattgccgatgtcggcaatgtagtgccgctccgcccacctctcaattaagtttttcagcgcgtggttgggaatgatctgcgctcatg
gggcgaaagaaggggttcagaggtgctttattgttactcgactgggcgtaccagcattcgtgcatgactgattatacatacaaaagtacagctc
gcttcaatgccctgcgattcctactcccgagcgagcactcctctcaccgtcgggttgcttcccacgaccacgccggtaagagggtctgtggcctc
gcgcccctcgcgagcgcatattccagccacgtctgtatgattttgcgctcatacgtctggcccgtcgaccccaaaatgacgggatcctgcataa
tatcgcccgaaatgggatccaggcattcgtcaggaggcgtcagccccgcgggagatgccggtcccgccgcattggaaaggtgtagagggggt

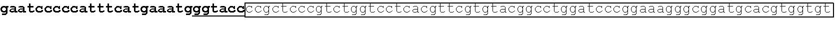

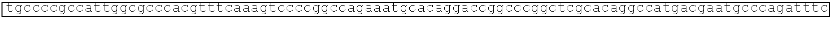

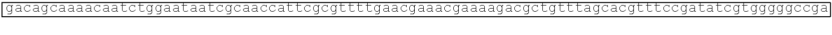

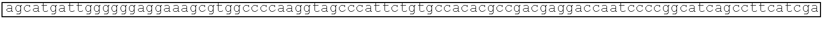

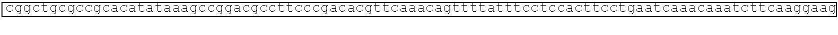

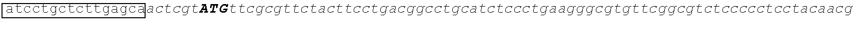

gcctgggcctgacgccccagatgggctgggacaactggaacacgttcgcctgcgacgtctccgagcagctgctgctggacacggccgacc
gcatctccgacctgggcctgaaggacatgggctacaagtacatcatcctggacgactgctggtcctccggccgcgactccgacggcttcctg
gtcgccgacgagcagaagttccccaacggcatgggccacgtcgccgaccacctgcacaacaactccttcctgttcggcatgtactcctccgc
gggcgagtacacgtgcgccggctaccccggctccctgggccgcgaggaggaggacgcccagttcttcgcgaacaaccgcgtggactacct
gaagtacgacaactgctacaacaagggccagttcggcacgcccgagatctcctaccaccgctacaaggccatgtccgacgccctgaacaa
gacgggccgccccatcttctactccctgtgcaactggggccaggacctgaccttctactggggctccggcatcgcgaactcctggcgcatgtc
cggcgacgtcacggcggagttcacgcgccccgactcccgctgcccctgcgacggcgacgagtacgactgcaagtacgccggcttccactgc
tccatcatgaacatcctgaacaaggccgcccccatgggccagaacgcgggcgtcggcggctggaacgacctggacaacctggaggtcgg
cgtcggcaacctgacggacgacgaggagaaggcgcacttctccatgtgggccatggtgaagtcccccctgatcatcggcgcgaacgtga
acaacctgaaggcctcctcctactccatctactcccaggcgtccgtcatcgccatcaaccaggactccaacggcatccccgccacgcgcgtct
ggcgctactacgtgtccgacacggacgagtacggccagggcgagatccagatgtggtccggccccctggacaacggcgaccaggtcgtg
gcgctgctgaacggcggctccgtgtcccgccccatgaacacgaccctggaggagatcttcttcgactccaacctgggctccaagaagctga
cctccacctgggacatctacgacctgtgggcgaaccgcgtcgacaactccacggcgtccgccatcctgggccgcaacaagaccgccaccg
gcatcctgtacaacgccaccgagcagtcctacaaggacggcctgtccaagaacgacacccgcctgttcggccagaagatcggctccctgtc

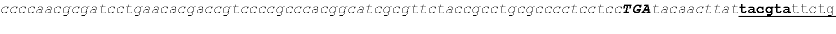

accggcgctgatgtggcgcggacgccgtcgtactctttcagactttactcttgaggaattgaacctttctcgcttgctggcatgtaaacattggcgc
aattaattgtgtgatgaagaaagggtggcacaagatggatcgcgaatgtacgagatcgacaacgatggtgattgttatgaggggccaaacctg
gctcaatcttgtcgcatgtccggcgcaatgtgatccagcggcgtgactctcgcaacctggtagtgtgtgcgcaccgggtcgctttgattaaaactg
atcgcattgccatcccgtcaactcacaagcctactctagctcccattgcgcactcgggcgcccggctcgatcaatgttctgagcggagggcgaag
cgtcaggaaatcgtctcggcagctggaagcgcatggaatgcggagcggagatcgaatcaggatcccgcgtctcgaacagagcgcgcagagga
acgctgaaggtctcgcctctgtcgcacctcagcgcggcatacaccacaataaccacctgacgaatgcgcttggttcttcgtccattagcgaagcgt

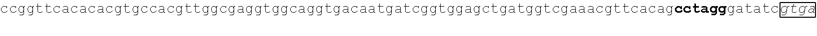

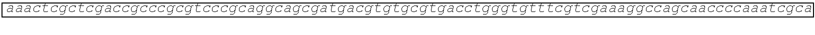

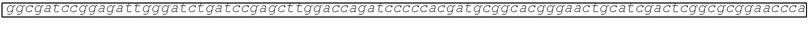

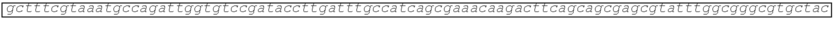

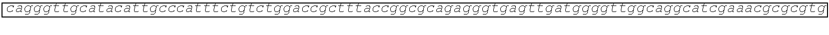

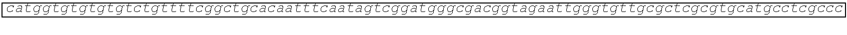

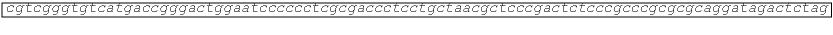

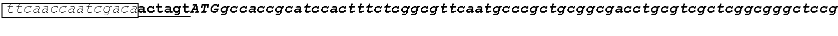

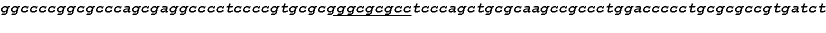

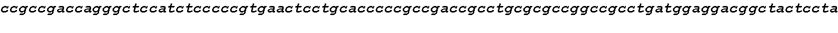

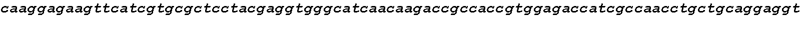

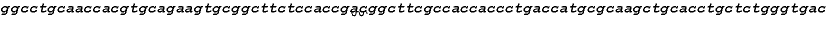

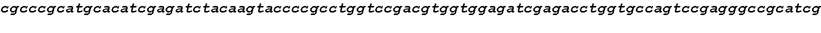

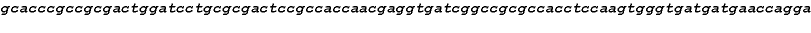

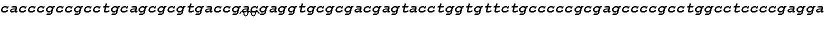

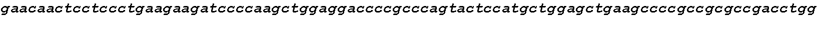

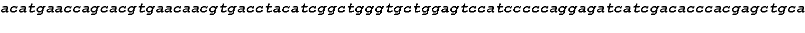

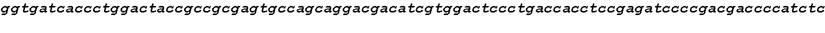

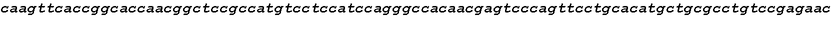

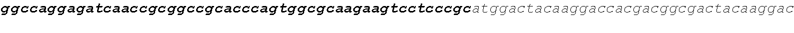

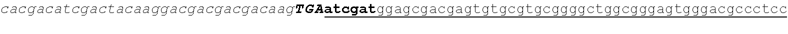

tcgctcctctctgttctgaacggaacaatcggccaccccgcgctacgcgccacgcatcgagcaacgaagaaaaccccccgatgataggttgcgg
tggctgccgggatatagatccggccgcacatcaaagggcccctccgccagagaagaagctcctttcccagcagactccttctgctgccaaaaca
cttctctgtccacagcaacaccaaaggatgaacagatcaacttgcgtctccgcgtagcttcctcggctagcgtgcttgcaacaggtccctgcacta
ttatcttcctgctttcctctgaattatgcggcaggcgagcgctcgctctggcgagcgctccttcgcgccgccctcgctgatcgagtgtacagtcaat
gaatggtgagctccgcgcctgcgcgaggacgcagaacaacgctgccgccgtgtcttttgcacgcgcgactccggcgcttcgctggtggcacccc
cataaagaaaccctcaattctgtttgtggaagacacggtgtacccccacccacccacctgcacctctattattggtattattgacgcgggagtgg
gcgttgtaccctacaacgtagcttctctagttttcagctggctcccaccattgtaaattcatgctagaatagtgcgtggttatgtgagaggtatag
tgtgtctgagcagacggggcgggatgcatgtcgtggtggtgatctttggctcaaggcgtcgtcgacgtgacgtgcccgatcatgagagcaatac
cgcgctcaaagccgacgcatagcctttactccgcaatccaaacgactgtcgctcgtatttfttggatatctattttaaagagcgagcacagcgcc
gggcatgggcctgaaaggcctcgcggccgtgctcgtggtgggggccgcgagcgcgtggggcatcgcggcagtgcaccaggcgcagacggag
gaacgcatggtgcgtgcgcaatataagatacatgtattgttgtcctgcagg
Nucleotide sequence of BnOTE (D124A) in pSZ6316:
SEQ ID NO: 132

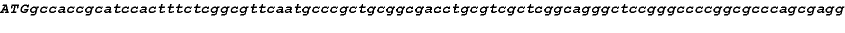

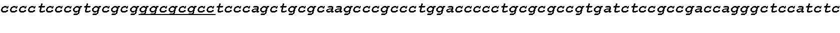

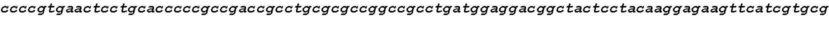

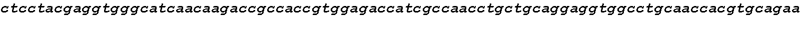

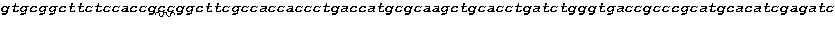

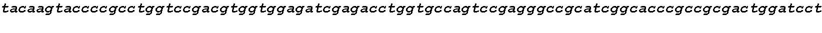

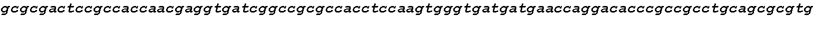

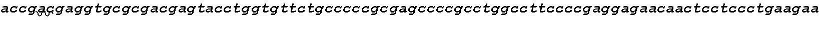

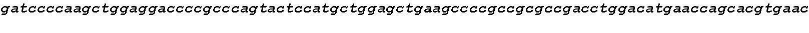

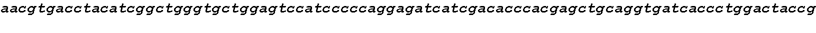

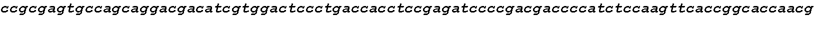

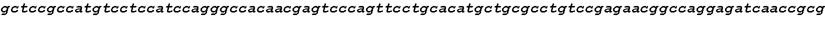

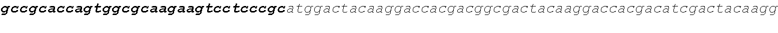

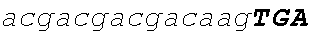

Nucleotide sequence of BnOTE (D209A) in pSZ6317:
SEQ ID NO: 133

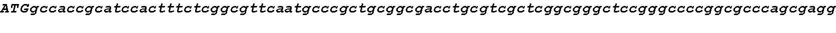

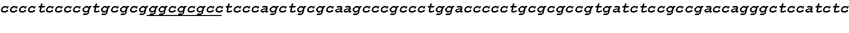

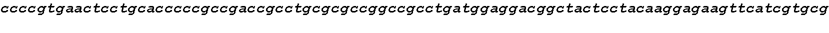

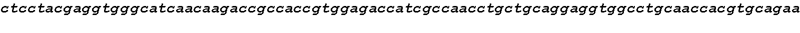

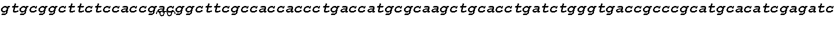

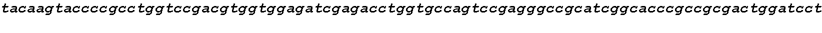

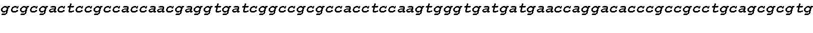

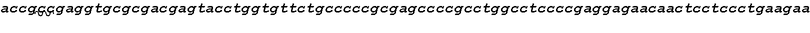

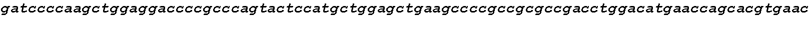

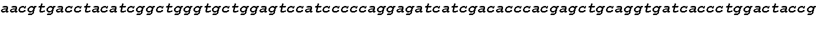

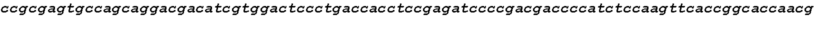

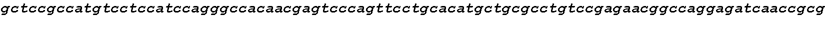

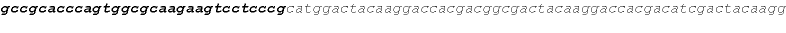

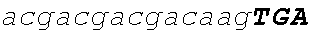

Nucleotide sequence of BnOTE (D124A, D209A) in pSZ6318
SEQ ID NO: 134

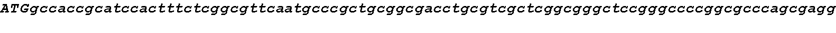

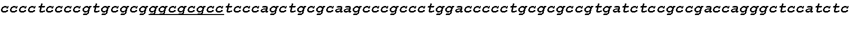

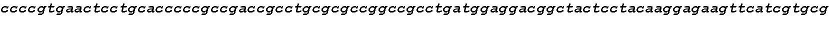

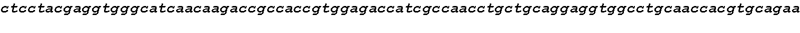

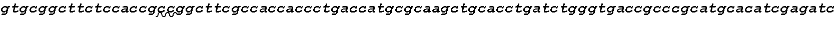

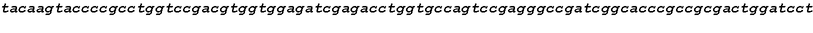

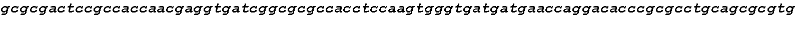

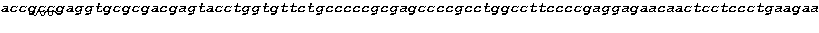

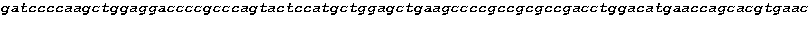

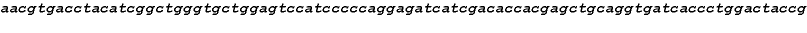

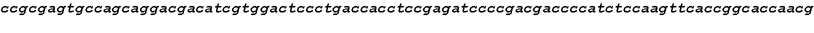

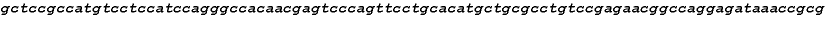

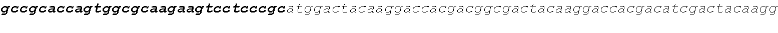

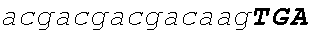

Nucleotide sequence of transforming DNA contained in pSZ5083
GarmGATA1
SEQ ID NO: 135
ccctcaactgcgacgctgggaaccttctccgggcaggcgatgtgcgtgggtttgcctccttg
gcacggctctacaccgtcgagtacgccatgaggcggtgatggctgtgtcggttgccacttcg
tccagagacggcaagtcgtccatcctctgcgtgtgtggcgcgacgctgcagcagtccctctg
cagcagatgagcgtgactttggccatttcacgcactcgagtgtacacaatccatttttctta
aagcaaatgactgctgattgaccagatactgtaacgctgatttcgctccagatcgcacagat
agcgaccatgttgctgcgtctgaaaatctggattccgaattcgaccctggcgctccatccat
gcaacagatggcgacacttgttacaattcctgtcacccatcggcatggagcaggtccactta
gattcccgatcacccacgcacatctcgctaatagtcattcgttcgtgtcttcgatcaatctc
aagtgagtgtgcatggatcttggttgacgatgcggtatgggtttgcgccgctggctgcaggg
tctgcccaaggcaagctaacccagctcctctccccgacaatactctcgcaggcaaagccggt
cacttgccttccagattgccaataaactcaattatggcctctgtcatgccatccatgggtct
gatgaatggtcacgctcgtgtcctgaccgttccccagcctctggcgtcccctgccccgccca
ccagcccacgccgcgcggcagtcgctgccaaggctgtctcggaGGTACCCTTTCTTGCGCTA
TGACACTTCCAGCAAAAGGTAGGGCGGGCTGCGAGACGGCTTCCCGGCGCTGCATGCAACAC
CGATGATGCTTCGACCCCCCGAAGCTCCTTCGGGGCTGCATGGGCGCTCCGATGCCGCTCCA
GGGCGAGCGCTGTTTAAATAGCCAGGCCCCCGATTGCAAAGACATTATAGCGAGCTACCAAA
GCCATATTCAAACACCTAGATCACTACCACTTCTACACAGGCCACTCGAGCTTGTGATCGCA
CTCCGCTAAGGGGGCGCCTCTTCCTCTTCGTTTCAGTCACAACCCGCAAACTCTAGAATATC
Aatgatcgagcaggacggcctccacgccggctcccccgccgcctgggtggagcgcctgttcg
gctacgactgggcccagcagaccatcggctgctccgacgccgccgtgttccgcctgtccgcc
cagggccgccccgtgctgttcgtgaagaccgacctgtccggcgccctgaacgagctgcagga
cgaggccgcccgcctgtcctggctggccaccaccggcgtgccctgcgccgccgtgctggacg
tggtgaccgaggccggccgcgactggctgctgctgggcgaggtgcccggccaggacctgctg
tcctcccacctggcccccgccgagaaggtgtccatcatggccgacgccatgcgccgcctgca
caccctggaccccgccacctgccccttcgaccaccaggccaagcaccgcatcgagcgcgccc
gcacccgcatggaggccggcctggtggaccaggacgacctggacgaggagcaccagggcctg
gcccccgccgagctgttcgcccgcctgaaggcccgcatgcccgacggcgaggacctggtggt
gacccacggcgacgcctgcctgcccaacatcatggtggagaacggccgcttctccggcttca
tcgactgcggccgcctgggcgtggccgaccgctaccaggacatcgccctggccacccgcgac
atcgccgaggagctgggcggcgagtgggccgaccgcttcctggtgctgtacggcatcgccgc
ccccgactcccagcgcatcgccttctaccgcctgctggacgagttcttctgaCAATTGACGC
CCGCGCGGCGCACCTGACCTGTTCTCTCGAGGGCGCCTGTTCTGCCTTGCGAAACAAGCCCC
TGGAGCATGCGTGCATGATCGTCTCTGGCGCCCCGCCGCGCGGTTTGTCGCCCTCGCGGGCG
CCGCGGCCGCGGGGGCGCATTGAAATTGTTGCAAACCCCACCTGACAGATTGAGGGCCCAGG
CAGGAAGGCGTTGAGATGGAGGTACAGGAGTCAAGTAACTGAAAGTTTTTATGATAACTAAC
AACAAAGGGTCGTTTCTGGCCAGCGAATGACAAGAACAAGATTCCACATTTCCGTGTAGAGG
CTTGCCATCGAATGTGAGCGGGCGGGCCGCGGACCCGACAAAACCCTTACGACGTGGTAAGA
AAAACGTGGCGGGCACTGTCCCTGTAGCCTGAAGACCAGCAGGAGACGATCGGAAGCATCAC
AGCACAGGATCCCGCGTCTCGAACAGAGCGCGCAGAGGAACGCTGAAGGTCTCGCCTCTGTC
GCACCTCAGCGCGGCATACACCACAATAACCACCTGACGAATGCGCTTGGTTCTTCGTCCAT
TAGCGAAGCGTCCGGTTCACACACGTGCCACGTTGGCGAGGTGGCAGGTGACAATGATCGGT
GGAGCTGATGGTCGAAACGTTCACAGCCTAGGGATATCGTGAAAACTCGCTCGACCGCCCGC
GTCCCGCAGGCAGCGATGACGTGTGCGTGACCTGGGTGTTTCGTCGAAAGGCCAGCAACCCC
AAATCGCAGGCGATCCGGAGATTGGGATCTGATCCGAGCTTGGACCAGATCCCCCACGATGC
GGCACGGGAACTGCATCGACTCGGCGCGGAACCCAGCTTTCGTAAATGCCAGATTGGTGTCC
GATACCTTGATTTGCCATCAGCGAAACAAGACTTCAGCAGCGAGCGTATTTGGCGGGCGTGC
TACCAGGGTTGCATACATTGCCCATTTCTGTCTGGACCGCTTTACCGGCGCAGAGGGTGAGT
TGATGGGGTTGGCAGGCATCGAAACGCGCGTGCATGGTGTGTGTGTCTGTTTTCGGCTGCAC
AATTTCAATAGTCGGATGGGCGACGGTAGAATTGGGTGTTGCGCTCGCGTGCATGCCTCGCC
CCGTCGGGTGTCATGACCGGGACTGGAATCCCCCCTCGCGACCCTCCTGCTAACGCTCCCGA
CTCTCCCGCCCGCGCGCAGGATAGACTCTAGTTCAACCAATCGACAACTAGTatggccaccg
catccactttctcggcgttcaatgcccgctgcggcgacctgcgtcgctcggcgggctccggg
ccccggcgcccagcgaggcccctccccgtgcgcgggcgcgccatccccccccgcatcatcgt
ggtgtcctcctcctcctccaaggtgaaccccctgaagaccgaggccgtggtgtcctccggcc
tggccgaccgcctgcgcctgggctccctgaccgaggacggcctgtcctacaaggagaagttc
atcgtgcgctgctacgaggtgggcatcaacaagaccgccaccgtggagaccatcgccaacct
gctgcaggaggtgggctgcaaccacgcccagtccgtgggctactccaccggcggcttctcca
ccacccccaccatgcgcaagctgcgcctgatctgggtgaccgcccgcatgcacatcgagatc
tacaagtaccccgcctggtccgacgtggtggagatcgagtcctggggccagggcgagggcaa
gatcggcacccgccgcgactggatcctgcgcgactacgccaccggccaggtgatcggccgcg
ccacctccaagtgggtgatgatgaaccaggacacccgccgcctgcagaaggtggacgtggac
gtgcgcgacgagtacctggtgcactgcccccgcgagctgcgcctggccttccccgaggagaa
caactcctccctgaagaagatctccaagctggaggacccctcccagtactccaagctgggcc
tggtgccccgccgcgccgacctggacatgaaccagcacgtgaacaacgtgacctacatcggc
tgggtgctggagtccatgccccaggagatcatcgacacccacgagctgcagaccatcaccct
ggactaccgccgcgagtgccagcacgacgacgtggtggactccctgacctcccccgagccct
ccgaggacgccgaggccgtgttcaaccacaacggcaccaacggctccgccaacgtgtccgcc
aacgaccacggctgccgcaacttcctgcacctgctgcgcctgtccggcaacggcctggagat
caaccgcggccgcaccgagtggcgcaagaagcccacccgcatggactacaaggaccacgacg
gcgactacaaggaccacgacatcgactacaaggacgacgacgacaagtgaATCGATgcagca
gcagctcggatagtatcgacacactctggacgctggtcgtgtgatggactgttgccgccaca
cttgctgccttgacctgtgaatatccctgccgcttttatcaaacagcctcagtgtgtttgat
cttgtgtgtacgcgcttttgcgagttgctagctgcttgtgctatttgcgaataccaccccca
gcatccccttccctcgtttcatatcgcttgcatcccaaccgcaacttatctacgctgtcctg
ctatccctcagcgctgctcctgctcctgctcactgcccctcgcacagccttggtttgggctc
cgcctgtattctcctggtactgcaacctgtaaaccagcactgcaatgctgatgcacgggaag
tagtgggatgggaacacaaatggaAAGCTTGAGCTCcagcgccatgccacgccctttgatgg
cttcaagtacgattacggtgttggattgtgtgtttgttgcgtagtgtgcatggtttagaata
atacacttgatttcttgctcacggcaatctcggcttgtccgcaggttcaaccccatttcgga
gtctcaggtcagccgcgcaatgaccagccgctacttcaaggacttgcacgacaacgccgagg
tgagctatgtttaggacttgattggaaattgtcgtcgacgcatattcgcgctccgcgacagc
acccaagcaaaatgtcaagtgcgttccgatttgcgtccgcaggtcgatgttgtgatcgtcgg
cgccggatccgccggtctgtcctgcgcttacgagctgaccaagcaccctgacgtccgggtac
gcgagctgagattcgattagacataaattgaagattaaacccgtagaaaaatttgatggtcg
cgaaactgtgctcgattgcaagaaattgatcgtcctccactccgcaggtcgccatcatcgag
cagggcgttgctcccggcggcggcgcctggctggggggacagctgttctcggccatgtgtgt
acgtagaaggatgaatttcagctggttttcgttgcacagctgtttgtgcatgatttgtttca
gactattgttgaatgtttttagatttcttaggatgcatgatttgtctgcatgcgact
Amino acid sequence of Gm FATA wild-type parental gene; D3997,
pSZ5083.
SEQ ID NO: 136
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
Amino acid sequence of Gm FATA S111A, V193A mutant gene; D3998, pSZ5084.
SEQ ID NO: 137
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFaTTPTMRKLRLIWVTARMHIEIYK
YPAWSDVVEI ESWGQGEGKIGTRRDWILRDYATGQVIGRATSKWVMMNQDTRRLQKVDaDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
Amino acid sequence of Gm FATA S111V, V193A mutant gene; D3999, pSZ5085.
SEQ ID NO: 138
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFvTTPTMRKLRLIWVTARMHIEIYK
YPAWSDVVEI ESWGQGEGKIGTRRDWILRDYATGQVIGRATSKWVMMNQDTRRLQKVDaDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
Amino acid sequence of Gm FATA G96A mutant gene; D4000,
pSZ5086.
SEQ ID NO: 139
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVaCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
Amino acid sequence of Gm FATA G96T mutant gene; D4001,
pSZ5087.
SEQ ID NO: 140
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVtCNHAQSVGYSTGGFSTTPTMRKLRLIWVTARMHIEIYK
YPAWSDVVEI ESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
Amino acid sequence of Gm FATA G96V mutant gene; D4002,
pSZ5088.
SEQ ID NO: 141
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVvCNHAQSVGYSTGGFSTT
YPAWSDVVEIESWGQGEGKIGTRRDWILRDYATGQVIGRATSKWVMMNQDTRRLQKVDVDVRDEYLVHCPRELRL
SKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
Amino acid sequence of GmFATA G108A mutant gene; D4003,
pSZ5089.
SEQ ID NO: 142
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTaGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
Amino acid sequence of GmFATA L91F mutant gene; D4004,
pSZ5090.
SEQ ID NO: 143
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANfLQEVGCDCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
Amino acid sequence of GmFATA L91K mutant gene; D4005,
pSZ5091.
SEQ ID NO: 144
MATASTFSAFNARCGDLRRSAGSGPRRPARPLPVRGRAIPPRIIVVSSSSKVNPLKTEAVVSSGLADRLRLGSL
TEDGLSYKEKFIVRCYEVGINKTATVETIANkLQEVGCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
Figure 10. Amino acid sequence of GmFATA L91S mutant gene;
D4006, pSZ5092. The algal transit peptide is underlined, the FLAG epitope tag is
uppercase bold and the L91S residue is lower-case cold
SEQ ID NO: 145
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANsLQEVGCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
Amino acid sequence of GmFATA G108V mutant gene; D4007, pSZ5093. The algal
transit peptide is underlined, the FLAG epitope tag is uppercase bold and the G108V residue is
lower-case bold.
SEQ ID NO: 146
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTvGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
Amino acid sequence of GmFATA T156F mutant gene; D4008,
pSZ5094. The algal transit peptide is underlined, the FLAG epitope tag is uppercase
bold and the T156F residue is lower-case bold.
SEQ ID NO: 147
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFSTTPTMRKLRLIWVTARMHIEIYK
YPAWSDVVEIESWGQGEGKIGfRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
Amino acid sequence of GmFATA T156A mutant gene; D4009,
pSZ5095. The algal transit peptide is underlined, the FLAG epitope tag is uppercase
bold and the T156A residue is lower-case bold.
SEQ ID NO: 148
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFSTTPTMRKLRLIWVTARMHIEIYK
YPAWSDVVEIESWGQGEGKIGaRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
Amino acid sequence of GmFATA T156K mutant gene; D4010, pSZ5096. The
algal transit peptide is underlined, the FLAG epitope tag is uppercase bold and the T156K residue
is lower-case bold.
SEQ ID NO: 149
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFSTTPTMRKLRLIWVTARMHIEIYK
YPAWSDVVEIESWGQGEGKIGkRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
Amino acid sequence of GmFATA T156V mutant gene; D4011, pSZ5097. The
algal transit peptide is underlined, the FLAG epitope tag is uppercase bold and the T156V residue
is lower-case bold.
SEQ ID NO: 150
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFSTTPTMRKLRLIWVTARMHIEIYK
YPAWSDVVEIESWGQGEGKIGvRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVFNHNGTNGSANVSANDHGCRNFLHLLRLSGNGLEINRGRTEWRKKPTRMDYKDHD
GDYKDHDIDYKDDDDK
Nucleotide sequence of the GmFATA S111A, V193A mutant gene (D3998,
pSZ5084). The promoter, 3′UTR, selection marker and targeting arms are the same as pSZ5083.
SEQ ID NO: 151
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttcgccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgcggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA s111V, V193A mutant gene (D3999,
pSZ5085). The promoter, 3′UTR, selection marker and targeting arms are the same as pSZ5083.
SEQ ID NO: 152
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttcgtcaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgcggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA G96A mutant gene (D4000, pSZ5086). The
promoter, 3′UTR, selection marker and targeting arms are the same as pSZ5083
SEQ ID NO: 153
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtggcgtgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA G96T mutant gene (D4001, pSZ5087). The
promoter, 3′UTR, selection marker and targeting arms are the same as pSZ5083
SEQ ID NO: 154
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgacgtgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA G96V mutant gene (D4002, pSZ5088). The
promoter, 3′UTR, selection marker and targeting arms are the same as pSZ5083
SEQ ID NO: 155
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtggtgtgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA G108A mutant gene (D4003, pSZ5089). The
promoter, 3′UTR, selection marker and targeting arms are the same as pSZ50836.
SEQ ID NO: 156
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccgcc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA L91F mutant gene (D4004, pSZ5090). The
promoter, 3′UTR, selection marker and targeting arms are the same as pSZ5083
SEQ ID NO: 157
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacttcctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA L91K mutant gene (D4005, pSZ5091).
SEQ ID NO: 158
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacaagctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA L91S mutant gene (D4006, pSZ5092).
SEQ ID NO: 159
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaactcgctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA G108V mutant gene (D4007, pSZ5093).
SEQ ID NO: 160
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccgtc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA T156F mutant gene (D4008, pSZ5094).
SEQ ID NO: 161
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcttccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA T156A mutant gene (D4009, pSZ5095)
SEQ ID NO: 162
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcgcgcgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA T156K mutant gene (D4010, pSZ5096).
SEQ ID NO: 163
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcaagcgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA T156V mutant gene (D4011, pSZ5097).
SEQ ID NO: 164
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcgtgcgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Amino acid sequence of wild type BnOTE in pSZ6315 (See SEQ ID NO: 131)
SEQ ID NO: 165
MATASTFSAFNARCGDLRRSAGSGPRRPARPLPVRGRASQLRKPALDPLRAVISADQGSISPVNSCTPADRLRAGR
LMEDGYSYKEKFIVRSYEVGINKTATVETIANLLQEVACNHVQKCGFSTDGFATTLTMRKLHLIWVTARMHIEIYKY
PAWSDVVEIETWCQSEGRIGTRRDWILRDSATNEVIGRATSKWVMMNQDTRRLQRVTDEVRDEYLVFCPREPRL
AFPEENNSSLKKIPKLEDPAQYSMLELKPRRADLDMNQHVNNVTYIGWVLESIPQEIIDTHELQVITLDYRRECQQD
DIVDSLTTSEIPDDPISKFTGTNGSAMSSIQGHNESQFLHMLRLSENGQEINRGRTQWRKKSSRMDYKDHDGDYK
DHDIDYKDDDDK
Amino Acid sequence of BnOTE (D124A) in pSZ6316 (See SEQ ID NO: 132)
SEQ ID NO: 166
MATASTFSAFNARCGDLRRSAGSGPRRPARPLPVRGRASQLRKPALDPLRAVISADQGSISPVNSCTPADRLRAGR
LMEDGYSYKEKFIVRSYEVGINKTATVETIANLLQEVACNHVQKCGFSTAGFATTLTMRKLHLIWVTARMHIEIYKYP
AWSDVVEIETWCQSEGRIGTRRDWILRDSATNEVIGRATSKWVMMNQDTRRLQRVTDEVRDEYLVFCPREPRLA
FPEENNSSLKKIPKLEDPAQYSMLELKPRRADLDMNQHVNNVTYIGWVLESIPQEIIDTHELQVITLDYRRECQQDD
IVDSLTTSEIPDDPISKFTGTNGSAMSSIQGHNESQFLHMLRLSENGQEINRGRTQWRKKSSRMDYKDHDGDYKD
HDIDYKDDDDK
Amino Acid sequence of BnOTE (D209A) in pSZ6317 (See SEQ ID NO: 133)
SEQ ID NO: 167
MATASTFSAFNARCGDLRRSAGSGPRRPARPLPVRGRASQLRKPALDPLRAVISADQGSISPVNSCTPADRLRAGR
LMEDGYSYKEKFIVRSYEVGINKTATVETIANLLQEVACNHVQKCGFSTDGFATTLTMRKLHLIWVTARMHIEIYKY
PAWSDVVEIETWCQSEGRIGTRRDWILRDSATNEVIGRATSKWVMMNQDTRRLQRVTAEVRDEYLVFCPREPRL
AFPEENNSSLKKIPKLEDPAQYSMLELKPRRADLDMNQHVNNVTYIGWVLESIPQEIIDTHELQVITLDYRRECQQD
DIVDSLTTSEIPDDPISKFTGTNGSAMSSIQGHNESQFLHMLRLSENGQEINRGRTQWRKKSSRMDYKDHDGDYK
DHDIDYKDDDDK
Amino acid sequence of BnOTE (D124A, D209A) in pSZ6318 (See SEQ ID NO: 134)
SEQ ID NO: 168
MATASTFSAFNARCGDLRRSAGSGPRRPARPLPVRGRASQLRKPALDPLRAVISADQGSISPVNSCTPADRLRAGR
LMEDGYSYKEKFIVRSYEVGINKTATVETIANLLQEVACNHVQKCGFSTAGFATTLTMRKLHLIWVTARMHIEIYKYP
AWSDVVEIETWCQSEGRIGTRRDWILRDSATNEVIGRATSKWVMMNQDTRRLQRVTAEVRDEYLVFCPREPRLA
FPEENNSSLKKIPKLEDPAQYSMLELKPRRADLDMNQHVNNVTYIGWVLESIPQEIIDTHELQVITLDYRRECQQDD
IVDSLTTSEIPDDPISKFTGTNGSAMSSIQGHNESQFLHMLRLSENGQEINRGRTQWRKKSSRMDYKDHDGDYKD
HDIDYKDDDDK
CpauLPAAT
SEQ ID NO: 169
MAIPAAAVIFLFGLLFFTSGLIINLFQALCFVLVWPLSKNAYRRINRVFAELLLS
ELLCLFDWWAGAKLKLFTDPETFRLMGKEHALVIINHMTELDWMLGWVMGQHLGC
LGS IL SVAKKS TKFLPVLGWSMWFSEYLYIERSWAKDRTTLKSHIERLTDYPLPF
WMVIFVEGTRFTRTKLLAAQQYAASSGLPVPRNVLIPRTKGFVSCVSHMRSFVPA
VYDVTVAFPKTSPPPTLLNLFEGQSIVLHVHIKRHAMKDLPESDDAVAQWCRDKF
VEKDALLDKHNAEDTFSGQEVHRTGSRPIKSLLVVISWVVVITFGALKFLQWSSW
KGKAFSVIGLGIVTLLMHMLILSSQAERSSNPAKVAQAKLKTELSISKKATDKEN
CprocLPAAT1i
SEQ ID NO: 170
MAIPAAAVIFLFGLIFFASGLIINLFQALCFVLIWPISKNAYRRINRVFAELLLS
ELLCLFDWWAGAKLKLFTDPETFRLMGKEHALVIINHMTELDWMVGWVMGQHFGC
LGSILSVAKKSTKFLPVLGWSMWFTEYLYIERSWNKDKSTLKSHIERLKDYPLPF
WLVIFAEGTRFTQTKLLAAQQYAASSGLPVPRNVLIPRTKGFVSCVSHMRSFVPA
VYDLTVAFPKTSPPPTLLNLFEGQSVVLHVHIKRHAMKDLPESDDEVAQWCRDKF
VEKDALLDKHNAEDTFSGQELQHTGRRPIKSLLVVISWVVVIAFGALKFLQWSSW
KGKAFSVIGLGIVTLLMHMLILSSQAERSKPAKVAQAKLKTELSISKTVTDKEN
CpcuLPAAT1
SEQ ID NO: 171
MAIPSAAVVFLFGLLFFTSGLIINLFQAFCFVLISPLSKNAYRRINRVFAELLPL
EFLWLFHWCAGAKLKLFTDPETFRLMGKEHALVIINHKIELDWMVGWVLGQHLGC
LGS ISVAKKSTKFLPVFGWSLWFSGYLFLERSWAKDKITLKSHIESLKDYPLPF
WLIIFVEGTRFTRTKLLAAQQYAASSGLPVPRNVLIPHTKGFVSSVSHMRSFVPA
IYDVTVAFPKTSPPPTMLKLFEGQSVELHVHIKRHAMKDLPESDDAVAQWCRDKF
VEKDALLDKHNSEDTFSGQEVHHVGRPIKALLVVISWVVVIIFGALKFLLWSSLL
SSWKGKAFSVIGLGIVAGIVTLLMHILILSSQAEGSNPVKAAPAKLKTELSSSKK
VTNKEN
ChookLPAAT1
SEQ ID NO: 172
MAIPSAAVVFLFGLLFFTSGLINLFQAFCFVLISPLSKNAYRRINRVFAELLPL
EFLWLFHWCAGAKLKLFTDPETFRLMGKEHALVIINHKIELDWMVGWVLGQHLGC
LGSILSVAKKSTKFLPVFGWSLWFSEYLFLERSWAKDKITLKSHIESLKDYPLPF
WLIIFVEGTRFTRTKLLAAQQYAASSGLPVPRNVLIPHTKGFVSSVSHMRSFVPA
IYDVTVAFPKTSPPPTMLKLFEGQSVELHVHIKRHAMKDLPESDDAVAQWCRDKF
VEKDALLDKHNSEDTFSGQEVHHVGRPIKALLVVISWVVVIIFGALKFLLWSSLL
SSWKGKAFSVIGLGIVAGIVTLLMHILILSSQAEGSNPVKAAPAKLKTELSSSKK
VTNKEN
CignLPAAT1
SEQ ID NO: 173
MAIAAAAVI FLFGLLFFASGIIINLFQALCFVLIWPLSKNVYRRINRVFAELLLM
DLLCLFHWWAGAKIKLFTDPETFRLMGMEHALVIMNHKIDLDWMVGWILGQHLGC
LGSILSIAKKSTKFIPVLGWSVWFSEYLFLERSWAKDKSTLKSHMEKLKDYPLPF
WLVIFVEGTRFTRTKLLAAQQYAASSGLPVPRNVLIPHTKGFVSCVSNMRSFVPA
VYDVTVAFPKSSPPPTMLKLFEGQSIVLHVHIKRHALKDLPESDDAVAQWCRDKF
VEKDALLDKHNAEDTFSGQEVHHIGRPIKSLLVVIAWVVVIIFGALKFLQWSSLL
STWKGKAFSVIGLGIATLLMHMLILSSQAERSNPAKVAK
CavigLPAAT1
SEQ ID NO: 174
MTIASAAVVFLFGILLFTSGLIINLFQAFCSVLVWPLSKNAYRRINRVFAEFLPL
EFLWLFHWWAGAKLKLFTDPETFRLMGKEHALVIINHKIELDWMVGWVLGQHLGC
LGSILSVAKKSTKFLPVFGWSLWFSEYLFLERNWAKDKKTLKSHIERLKDYPLPF
WLIIFVEGTRFTRTKLLAAQQYAASAGLPVPRNVLIPHTKGFVSSVSHMRSFVPA
IYDVTVAFPKTSPPPTMLKLFEGHFVELHVHIKRHAMKDLPESEDAVAQWCRDKF
VEKDALLDKHNAEDTFSGQEVHHVGRPIKSLLVVISWVVVIIFGALKFLQWSSLL
SSWKGIAFSVIGLGTVALLMQILILSSQAERSIPAKETPANLKTELSSSKKVTNK
EN
CavigLPAAT2
SEQ ID NO: 175
MAIAAAAVIVPVSLLFFVSGLIVNLVQAVCFVLIRPLFKNTYRRINRVVAELWL
ELVWLIDWWAGVKIKVFTDHETFHLMGKEHALVICNHKSDIDWLVGWVLAQRSGC
LGSTLAVMKKSSKFLPVIGWSMWFSEYLFLERNWAKDESTLKSGLNRLKDYPLPF
WLALFVEGTRFTRAKLLAAQQYAASSGLPVPRNVLIPRTKGFVSSVSHMRSFVPA
IYDVTVAI PKTSPPPTLLRMFKGQSSVLHVHLKRHQMNDLPESDDAVAQWCRDIF
VEKDALLDKHNAEDTFSGQELQDTGRPIKSLLIVISWAVLVVFGAVKFLQWSSLL
SSWKGLAFSGIGLGVITLLMHILILFSQSERSTPAKVAPAKPKIEGESSKTEMEK
EH
CpalLPAAT1
SEQ ID NO: 176
MAIAAAAVIVPLGLLFFVSGLIVNLVQAVCFVLIRPLSKNTYRRINRVVAELLWL
ELVWLIDWWAGVKIKVFTDHETLSLMGKEHALVICNHKSDIDWLVGWVLAQRSGC
LGSTLAVMKKSSKFLPVIGWSMWFSEYLPESDDAVAQWCRDIFVEKDALLDKHNA
EDTFSGQELQDTGRPIKSLLVVISWAVLVIFGAVKFLQWSSLLSSWKGLAFSGVG
LGIITLLMHILILFSQSERSTPAKVAPAKPKKDGESSKTEIEKENVPGALLGQGR
EHPEVRPEPPEGLPPALLAGPVRGGHPLHPRQAAGRPAVRHLLRPARAPQRADPP
HQGLRVLRVPHALLRARHLRRDRGHPQDLPPPHHAAHVQGPVLRAARAPEAPPDE
GP
CuPSR23 LPAAT2
SEQ ID NO: 177
MAIAAAAVI FLFGLIFFASGLIINLFQALCFVLIRPLSKNAYRRINRVFAELLLS
ELLCLFDWWAGAKLKLFTDPETFRLMGKEHALVIINHMTELDWMVGWVMGQHFGC
LGSIISVAKKSTKFLPVLGWSMWFSEYLYLERSWAKDKSTLKSHIERLIDYPLPF
WLVIFVEGTRFTRTKLLAAQQYAVSSGLPVPRNVLIPRTKGFVSCVSHMRSFVPA
VYDVTVAFPKTSPPPTLLNLFEGQSIMLHVHIKRHAMKDLPESDDAVAEWCRDKF
VEKDALLDKHNAEDTFSGQEVCHSGSRQLKSLLVVISWVVVTTFGALKFLQWSSW
KGKAFSAIGLGIVTLLMHVLILSSQAERSNPAEVAQAKLKTGLSISKKVTDKEN
CkoeLPAAT1
SEQ ID NO: 178
MAI PAAVAVI PIGLLFIISGLIVNLIQAVVYVLIRPLSKNLHRKINKPIAELLWL
ELIWLVDWWAGIKVEVYADSQTLELMGKEHALLICNHRSDIDWLVGWVLAQRARC
LGSALAIMKKSAKFLPVIGWSMWFSDYIFLDRTWAKDEKTLKSGFERLADFPMPF
WLALFVEGTRFTKAKLLAAQEYAASRGLPVPQNVLIPRTKGFVTAVTHMRSYVPA
IYDCTVDISKAHPAPSILRLIRGQSSVVKVQITRHSMQELPETADGISQWCMDLF
VTKDGFLEKYHSKDIFGSLPVQNIGRPVKSLIVVLCWYCLMAFGLFKFFMWSSLL
SSWEGILSLGLILLAVAIVMQILIQSTESERSTPVKSIQKDPSKETLLQN
CkoeLPAAT2
SEQ ID NO: 179
MHVLLEMVTFRFSSFFVFDNVQALCFVLIWPLSKSAYRKINRVFAELLLSELLCL
FDWWAGAKLKLFTDPETFRLMGKEHALVITNHKIDLDWMIGWILGQHFGCLGSVI
SIAKKSTKFLPIFGWSLWFSEYLFLERNWAKDKRTLKSHIERMKDYPLPLWLILF
VEGTRFTRTKLLAAQQYAASSGLPVPRNVLIPHTKGFVSSVSHMRSFVPAVYDVT
VAFPKTSPPPTMLSLFEGQSVVLHVHIKRHAMKDLPDSDDAVAQWCRDKFVEKDA
LLDKHNAEDTFSGQEVHHVGRTIKSLLVVISWMVVIIFGALKFLQWSSLLSSWKG
KAFSAIGLGIATLLMHVLVVFSQADRSNPAKVPPAKLNTELSSSKKVTNKEN
CprocLPAAT2
SEQ ID NO: 180
MAI PAAVAVIPIGLLFIISGLIVNLIQAVVYVLIRPLSKNLYRKINKPIAELLWL
ELIWLVDWWAGIKVEVYADSETLESMGKEHALLICNHRSDIDWLVGWVLAQRARC
LGSALAIMKKSAKFLPVIGWSMWFSDYIFLDRTWEKDEKTLKSGFERLADFPMPF
WLALFVEGTRFTKAKLLAAQEFAASRGLPVPQNVLIPRTKGFVTAVTHMRSYVPA
IYDCTVDISKAHPAPSILRLIRGQSSVVKVQITRHSMQELPETPDGISQWCMDLF
VTKDAFLEKYHSKDIFGSLPVHDIGRPVKSLIVVLCWYSLMAFGFYKFFMWSSLL
SSWEGILSLGLVLIVIAIVMQILIQSSESERSTPVKSVQKDPSKETLLQN
CavigGPAT9
SEQ ID NO: 181
MATGGSLKPSSSDLDLDHPNIEDYLPSGSSINEPAGKLRLRDLLDISPTLTEAAG
AIVDDSFTRCFKSIPREPWNWNLYLFPLWCIGVLIRYFILFPGRVIVLTMGWITV
ISSFIAVRVLLKGHDALQIKLERLIVQLLCSSFVASWTGVVKYHGPRPSIRPKQV
YVANHT SMIDFFILDQMTVFSVIMQKHPGWVGLLQSTLLESVGCIWFDRAEAKDR
GIVAKKLWDHVHGEGNNPLLIFPEGTCVNNNYSVMFKKGAFELGCTVCPVAIKYN
KIFVDAFWNSKKQSFTRHLLQLMTSWAVVCDVWYLEPQTLKPGETPIEFAERVRD
IISARAGLKKVPWDGYLKYSRPSPKHRERKQQTFAESVLQRLEE
ChookGPAT9-1
SEQ ID NO: 182
MATAGSLKPSRSELDFDRPNIEDYLPSGSSIIEPAGKLRLRDLLDISPTLTEAAG
AIVDDSFTRCFKSNPPEPWNWNIYLFPLWCFGVLIRYLILFPARVIVLTIGWIIF
LSSFIPVHLLLKGHDALRI KLERLLVELICSFFVASWTGVVKYHGPRPSIRPKQV
YVANHTSMIDFFILDQMTVFSVIMQKHPGWVGLLQSTLLESVGCIWFDRAEAKDR
GIVAKKLWDHVHGEGNNPLLIFPEGTCVNNNYSVMFKKGAFELGCTVCPVAIKYN
KIFVDAFWNSKKQSFTRHLLQLMTSWAVVCDVWYLEPQTLKPGETPIEFAERVRD
IISVRAGLKKVPWDGYLKYSRPSPKHTERKQQNFAESVLQRLEKK
CignGPAT9-1
SEQ ID NO: 183
MATGGRLKPSSSELDLDRANTEDYLPSGSSINEPVGKLRLRDLLDISPTLTEAAG
AIVDDSFTRCFKSIPPEPWNWNIYLFPLWCFGVLIRYFILFPARVIVLTIGWITV
ISSFTAVRFLLKGHNALQIKLERLIVQLLCSSFVASWTGVVKYHGPRPSIRPKQV
YVANHTSMIDFLILDQMTVFSVIMQKHPGWVGLLQSTLLESVGCIWFNRAEAKDR
EIVAKKLWDHVHGEGNNPLLIFPEGTCVNNHYSVMFKKGAFELGCTVCPVAIKYN
KIFVDAFWNSRKQSFTMHLLQLMTSWAVVCDVWYLEPQTLKPGETAIEFAERVRD
IIVRAGLKKVPWDGYLKYSRPSPKHRESKQQSFAESVLRRLEEK
CignGPAT9-2
SEQ ID NO: 184
MATGGRLKPSSSELDLDRANTEDYLPSGSSINEPVGKLRLRDLLDISPTLTEAAG
AIVDDSFTRCFKSIPPEPWNWNIYLFPLWCFGVLIRYFILFPARVIVLTIGWITV
ISSFTAVRFLLKGHNALQIKLERLIVQLLCSSFVASWTGVVKYHGPRPSIRPKQV
YVANHTSMIDFLILDQMTVFSVIMQKHPGWVGLLQSTLLESVGCIWFNRAEAKDR
EIVAKKLWDHVHGEGNNPLLI FPEGTCVNNHYSVMFKKGAFELGCTVCPVAIKYN
KIFVDAFWNSKKHSFTRHLLQLMTSWAVVCDVWYLEPQTLKPGETPIEFAERVRD
IISVRADLKKVPWDGYLKYSRPSPKHRERKQQKFAESVLRRLEEK
CpalGPAT9-1
SEQ ID NO: 185
MATAGRLKPSSSELELDLDRPNIEDYLPSGSSINEPAGKLRLRDLLDISPMLTEA
AGAIVDDSFTRCFKSIPPEPWNWNIYLFPLWCFGVLIRYLILFPARVIVLTVGWI
TVISSFITVRFLLKGHDSLRIKLERLIVQLFCSSFVASWTGVVKYHGPRPSIRPQ
QVYVANHTSMIDFIILNQMTVFSAIMQKHPGWVGLIQSTILESVGCIWFNRAEAK
DREIVAKKLLDHVHGEGNNPLLIFPEGTCVNNHYSVMFKKGAFELGCTVCPVAIK
YNKIFVDAFWNSKKQSFTMHLLQLMTSWAVVCDVWYLEPQTLKPGETPIEFAERV
RDIISVRAGLKKVPWDGYLKYSRPSPKHRERKQQSFAESVLRRLEKR
CpalGPATt9-2
SEQ ID NO: 186
MATAGRLKPSSSELELDLDRPNIEDYLPSGSSINEPAGKLRLRDLLDISPMLTEA
AGAIVDDSFTRCFKSIPPEPWNWNIYLFPLWCFGVLIRYLILFPARVIVLTVGWI
TVISSFITVRFLLKGHDSLRIKLERLIVQLFCSSFVASWTGVVKYHGPRPSIRPQ
QVYVANHTSMIDFIILNQMTVFSAIMQKHPGWVGLIQSTILESVGCIWFNRAEAK
DREIVAKKLLDHVHGEGNNPLLIFPEGTCVNNHYSVMFKKGAFELGCTVCPVAIK
YNKIFVDAFWNSKKLSFTMHLLQLMTSWAVVCDVWYLEPQTLKPGETPIEFAERV
RDIISVRAGLKKVPWDGYLKYSRPSPKHRERKQQTFAESVLRRLEEKGNVVPTVN
CavigDGAT1
SEQ ID NO: 187
MAIADGGIIGAAGSISALTADTDPPSLRRRNVPAGQASAVSAFSTESMAKHLCDP
SREPSPSPKSSDDGKDPDIGSVDSLNEKPSSPAAGKGRLQHDLRFTYRASSPAHR
KVKESPLSSSNIFKQSHAGLFNLCVVVLVAVNSRLIIENLMKYGLLIKTGFWFSS
RSLRDWPLFMCCLSLPIFPLAAFLVEKLAQKNRLQEPTVVCCHVLITSVSILYPV
LVILRCDSAVLSGVALMLFACIVWLKLVSYAHSNYDMRYVAKSLDKGEPVVDSVI
ADHPYRVDYKDLVYFMVAPTLCYQLSYPLTPCVRKSWIARQVMKLVLFTGVMGFI
VEQYINP IVQNSKHPLKGDLLYAIERVLKLSVPNLYVWLCMFYCFFHLWLNILAE
LICFGDREFYKDWWNAKTVEEYWRMWNMPVHKWMVRHIYFPCLRNGIPRGVAVLI
AFLVSAVFHELCIAVPCHVFKLWAFIGIMFQVPLVLVSNCLQKKFQSSMAGNMFF
WFIFCIFGQPMCVLLYYHDLMNRKGSRID
ChookDGAT1-1
SEQ ID NO: 188
MAIADGGSAGAAGSISGSDPSPSTAPSLRRRNASAGQAFSTESMARDLCDPSREP
SLSPKSSDDGKDPADDIGAADSVDSGGVKDEKPSSQAAAKARLEHDLRFTYRASS
PAHRKVKESPLSSSNIFKQSHAGLFNLCVVVLVAVNSRLIIENLMKYGLLIKTGF
WFSSRSLRDWPLFMCCLSLPIFPLAAFLVEKLAQKNRLQEPTVVCCHVIITSVSI
LYPVLVILRCDSAVLSGVALMLFACIVWLKLVSYAHANYDMRSVAKSLDKGETVA
DSVIVDHPYRVDYKDLVYFMVAPTLCYQLSYPLTPYVRKSWVARQVMKLVLFTGV
MGFIVEQYINPIVQNSKHPLKGDLLYAIERVLKLSVPNLYVWLCMFYCFFHLWLN
ILAELTCFGDREFYKDWWNAKTVEEYWRMWNMPVHKWMVRHIYFPCLRNGIPRGV
AVLIAFLVSAVFHELCIAVPCHVFKLWAFIGIMFQVPLVLVSNCLQKKFQSSMAG
NMFFWFIFCIFGQPMCVLLYYHDLMNRKGSRID
CavigLPCAT
SEQ ID NO: 189
MGLVSVAAAIGVSVPVARFLLCFLATIPVSFLWRLVPGRLPKHLYSAASGAILSY
LSFGASSNLHFIVPMTLGYLSMLFFRPFSGLLTFFLGFGYLIGCHVYYMSGDAWK
EGGIDATGALMVLTLKVISCSMNYNDGLLKEEGLRESQKKNRLTKMPSLIEYFGY
CLCCGSHFAGPVYEMKDYLEWTEGKGIWSRSQKEPKPSPFGGALRAIIQAAVCMA
MYLYLVPHHPLTRFTEPVYYEWGFFRRLSYQYMAALTARWKYYFIWSISEASLII
SGLGFSGWTESSPPKPRWDRAKNVDIIGVEFAKSSVQLPLVWNIQVSIWLRHYVY
DRLVQNGKRPGFFQLLATQTVSAVWHGLYPGYIIFFVQSALMIAGSRVIYRWQQA
VPPKMGLVKNIFVFFNFAYTLLVLNYSAVGFMVLSMHETLASYGSVYYIGTILPI
TLILLSYVIKPGKPARSKAHKEQ
CpalLPCAT
SEQ ID NO: 190
MELGSVAAAIGVSVPVARFLLCFLATIPVSFLWRLVPGRLPKHLYSAASGAILSY
LSFGPSSNLHFIVPMTLGYLSMLFFRFSGLLTFFLGFGYLIGCHVYYMSGDAWK
EGGIDATGALMVLTLKVISCSINYNDGLLKEEGLRESQKKNRLTKMPSLIEYIGY
CLCCGSHFAGPVYEMKDYLEWTEGKGVWSHSEKEPKPSPFGGALRAIIQAAVCMA
MYMYLVPHHPLSRFTEPVYYEWGFFRRLSYQYMAGLTARWKYYFIWSISEASLII
SGLGFSGWTESSPPKPRWDRAKNVDIIGVEFAKSSVQLPLVWNIQVSTWLRHYVY
DRLVQNGKRPGFFQLLATQTVSAIWHGLYPGYIIFFVQSALMIAGSRVIYRWQQA
VPPKMGLVKNIFVFFNFAYTLLVLNYSAVGFMVLSMHETLASYGSVYYIGTILPI
TLILLSYVIKPGKPARSKAHKEQ
CpauLPCAT
SEQ ID NO: 191
MELEIGSVAAAIGVSVPVARFLLCFLATIPVSFLCRLLPARLPKHLYSAASGAIL
SYLSFGPSSNLHFIVPMSLGYLSMLFFRPFSGLLTFFLGFGYLIGCHVYYMSGDA
WKEGGIDATGALMVLTLKVISCSINYNDGLLKEEGLRESQKKNRLTKMPSLIEYF
GYCLCCGSHFAGPVYEMKDYLEWTEGKGIWSRSEKDPKPSPFGGALRAIIQAAVC
MAMHMYLVPHHPLTRFTEPVYYEWGFFRRLSYQYMAAQTARWKYYFIWSISEASL
IISGLGFSGWTESSPPKPRWDKAKNVDIIGVEFAKSSVQLPLVWNIQVSTWLRHY
VYDRLVQNGKRPGFFQLLATQTVSAVWHGLYPGYIIFFVQSALMIAGSRVIYRWQ
QAVPQKMGLVKNIFVFFNFAYTLLVLNYSAVGFMVLSMHETLASYGSVYYIGTIL
PITLILLSYVIKPGKPTRSKVHKEQ
CschuLPCAT
SEQ ID NO: 192
MELEMEPLAAAIGVSVAVFRFLVCFIATIPVSFICRLVPGGLPRHLFSAASGAVL
SYLSFGFSSNLHFLVPMTLGYLSMILFRRFCGILTFFLGFGYLIGCHVYYMSGDA
WKEGGIDATGALMVLTLKVISCSINYNDGLLKEEGLRESQKKNRLIRLPSLIEYF
GYCLCCGSHFAGPVYEMKDYLDWTEGKGIWSHSEKGPKPSPLRAALRAIIQAGFC
MAMYLYLVPHYPLTRFTDPVYYEWGILRRLSYQYMASFTARWKYYFIWSISEASL
IISGLGFSGWTESSPPKPRWDRAKNVDILGVELAKSSVQIPLVWNIQVSTWLRHY
VYDRLVQNGKRPGFLQLLATQTVSAIWHGVYPGYLIFFVQSALMIAGSRAIYRWQ
QAVPPKMSLVKNTLVFFNFAYTLLVLNYSAVGFMVLSMHETLASYGSVYYVGTIL
PVTLILLGYVIKPGKSPRSKASKEQ
CawgPLA2-1
SEQ ID NO: 193
MNFDFLSNIPWFGAKASDNAGSSFGSATIVIQQPPPVSRGFDIRHWGWPWSVLSV
LPWGKPGCDELRAPPTTINRRLKRNATSMHSSAVRGNAEAARVRFRPYVSKVPWH
TGFRGLLSQLFPRYGHYCGPNWSSGKNGGSPVWDQRPIDWLDYCCYCHDIGYDTH
DQAKLLEADLAFLECLERPSYPTKGDAHVAHMYKTMCVTGLRNVLIPYRTQLLRL
NSRQPLIDFGWLSNAAWKGWNAQKS
CignPLA2-1
SEQ ID NO: 194
MNLDFLSKI PWFEAKASENPGLNLGSTTIVIKQPRQGFDIRHWGWPWSVLTWGNR
VTDEVHAPPTTINRRLKRNATGPAVQGDTEAARLRFRPYVSKVPWHTGFRGLLSQ
LFPRYGHYCGPNWSSGKNGGSPVWDQRPIDWLDYCCYCHDIGYDTHDQAKLLEAD
LAFLECLERPSYPTTGDAHVAHMYKTMCVTGLRNVLIPYRTQLLRLNFRQPLIDF
GWLSNAAWKGWSAQKT
CuPSR23PLA2-2
SEQ ID NO: 195
MVHLPHTLKLGLVIAISISGLCFSSTPARALNVGIQAAGVTVSVGKGCSRKCESD
FCKVPPFLRYGKYCGLMYSGCPGEKPCDGLDACCMKHDACVQAKNNDYLSQECSQ
NLLNCMASFRMSGGKQFKGSTCQVDEVVDVLTVVMEAALLAGRYLHKP
CprocPLA2-2
SEQ ID NO: 196
MVHLPHTLKLGLVIAISISGLCLSSTPARALNVGIQAAGVTVSVGKGCSRKCESD
FCKVPPFLRYGKYCGLMYSGCPGEKPCDGLDACCMKHDACVQAKNDDYLSQECSQ
NLLNCMASFRMSGGKQFKGSTCQVDEVVDVLTVVMEAALLAGRYLHKP
pSZ4329
SEQ ID NO: 197
agcggaagagcgcccaatgtttaaacccctcaactgcgacgctgggaaccttctccgggcaggcgatgtgcgtgggtttgcctccttggcacgg
ctctacaccgtcgagtacgccatgaggcggtgatggctgtgtcggttgccacttcgtccagagacggcaagtcgtccatcctctgcgtgtgtggc
gcgacgctgcagcagtccctctgcagcagatgagcgtgactttggccatttcacgcactcgagtgtacacaatccatttttcttaaagcaaatga
ctgctgattgaccagatactgtaacgctgatttcgctccagatcgcacagatagcgaccatgttgctgcgtctgaaaatctggattccgaattcg
accctggcgctccatccatgcaacagatggcgacacttgttacaattcctgtcacccatcggcatggagcaggtccacttagattcccgatcacc
cacgcacatctcgctaatagtcattcgttcgtgtcttcgatcaatctcaagtgagtgtgcatggatcttggttgacgatgcggtatgggtttgcgc
cgctggctgcagggtctgcccaaggcaagctaacccagctcctctccccgacaatactctcgcaggcaaagccggtcacttgccttccagattg
ccaataaactcaattatggcctctgtcatgccatccatgggtctgatgaatggtcacgctcgtgtcctgaccgttccccagcctctggcgtcccct
gccccgcccaccagcccacgccgcgcggcagtcgctgccaaggctgtctcggaggtaccctttcttgcgctatgacacttccagcaaaaggtag
ggcgggctgcgagacggcttcccggcgctgcatgcaacaccgatgatgcttcgaccccccgaagctccttcggggctgcatgggcgctccgatg
ccgctccagggcgagcgctgtttaaatagccaggcccccgattgcaaagacattatagcgagctaccaaagccatattcaaacacctagatca
ctaccacttctacacaggccactcgagcttgtgatcgcactccgctaagggggcgcctcttcctcttcgtttcagtcacaacccgcaaactctaga
atatcaatgctgctgcaggccttcctgttcctgctggccggcttcgccgccaagatcagcgcctccatgacgaacgagacgtccgaccgccccct
ggtgcacttcacccccaacaagggctggatgaacgaccccaacggcctgtggtacgacgagaaggacgccaagtggcacctgtacttccagt
acaacccgaacgacaccgtctgggggacgcccttgttctggggccacgccacgtccgacgacctgaccaactgggaggaccagcccatcgcc
atcgccccgaagcgcaacgactccggcgccttctccggctccatggtggtggactacaacaacacctccggcttcttcaacgacaccatcgacc
cgcgccagcgctgcgtggccatctggacctacaacaccccggagtccgaggagcagtacatctcctacagcctggacggcggctacaccttca
ccgagtaccagaagaaccccgtgctggccgccaactccacccagttccgcgacccgaaggtcttctggtacgagccctcccagaagtggatca
tgaccgcggccaagtcccaggactacaagatcgagatctactcctccgacgacctgaagtcctggaagctggagtccgcgttcgccaacgagg
gcttcctcggctaccagtacgagtgccccggcctgatcgaggtccccaccgagcaggaccccagcaagtcctactgggtgatgttcatctccatc
aaccccggcgccccggccggcggctccttcaaccagtacttcgtcggcagcttcaacggcacccacttcgaggccttcgacaaccagtcccgcg
tggtggacttcggcaaggactactacgccctgcagaccttcttcaacaccgacccgacctacgggagcgccctgggcatcgcgtgggcctccaa
ctgggagtactccgccttcgtgcccaccaacccctggcgctcctccatgtccctcgtgcgcaagttctccctcaacaccgagtaccaggccaacc
cggagacggagctgatcaacctgaaggccgagccgatcctgaacatcagcaacgccggcccctggagccggttcgccaccaacaccacgttg
acgaaggccaacagctacaacgtcgacctgtccaacagcaccggcaccctggagttcgagctggtgtacgccgtcaacaccacccagacgat
ctccaagtccgtgttcgcggacctctccctctggttcaagggcctggaggaccccgaggagtacctccgcatgggcttcgaggtgtccgcgtcct
ccttcttcctggaccgcgggaacagcaaggtgaagttcgtgaaggagaacccctacttcaccaaccgcatgagcgtgaacaaccagcccttca
agagcgagaacgacctgtcctactacaaggtgtacggcttgctggaccagaacatcctggagctgtacttcaacgacggcgacgtcgtgtcca
ccaacacctacttcatgaccaccgggaacgccctgggctccgtgaacatgacgacgggggtggacaacctgttctacatcgacaagttccagg
tgcgcgaggtcaagtgacaattgacgcccgcgcggcgcacctgacctgttctctcgagggcgcctgttctgccttgcgaaacaagcccctggag
catgcgtgcatgatcgtctctggcgccccgccgcgcggtttgtcgccctcgcgggcgccgcggccgcgggggcgcattgaaattgttgcaaacc
ccacctgacagattgagggcccaggcaggaaggcgttgagatggaggtacaggagtcaagtaactgaaagtttttatgataactaacaaca
aagggtcgtttctggccagcgaatgacaagaacaagattccacatttccgtgtagaggcttgccatcgaatgtgagcgggcgggccgcggacc
cgacaaaacccttacgacgtggtaagaaaaacgtggcgggcactgtccctgtagcctgaagaccagcaggagacgatcggaagcatcacag
cacaggatcccgcgtctcgaacagagcgcgcagaggaacgctgaaggtctcgcctctgtcgcacctcagcgcggcatacaccacaataacca
cctgacgaatgcgcttggttcttcgtccattagcgaagcgtccggttcacacacgtgccacgttggcgaggtggcaggtgacaatgatcggtgg
agctgatggtcgaaacgttca cagcctagggatatcgcctgctcaagcgggcgctcaacatgcagagcgtcagcgagacgggctgtggcgat
cgcgagacggacgaggccgcctctgccctgtttgaactgagcgtcagcgctggctaaggggagggagactcatccccaggctcgcgccaggg
ctctgatcccgtctcgggcggtgatcggcgcgcatgactacgacccaacgacgtacgagactgatgtcggtcccgacgaggagcgccgcgagg
cactcccgggccaccgaccatgtttacaccgaccgaaagcactcgctcgtatccattccgtgcgcccgcacatgcatcatcttttggtaccgactt
cggtcttgttttacccctacgacctgccttccaaggtgtgagcaactcgcccggacatgaccgagggtgatcatccggatccccaggccccagc
agcccctgccagaatggctcgcgctttccagcctgcaggcccgtctcccaggtcgacgcaacctacatgaccaccccaatctgtcccagacccc
aaacaccctccttccctgcttctctgtgatcgctgatcagcaacaactagtaacaatggccaccgcctccaccttctccgccttcaacgcccgctg
cggcgacctgcgccgctccgccggctccggcccccgccgccccgcccgccccctgcccgtgcgcgccgccatcaacgactccgcccaccccaag
gccaacggctccgccgtgagcctgaagagcggcagcctgaacacccaggaggacacctcctccagcccccccccccgcaccttcctgcaccag
ctgcccgactggagccgcctgctgaccgccatcaccaccgtgttcgtgaagtccaagcgccccgacatgcacgaccgcaagtccaagcgcccc
gacatgctggtggacagcttcggcctggagtccaccgtgcaggacggcctggtgttccgccagtccttctccatccgctcctacgagatcggcac
cgaccgcaccgccagcatcgagaccctgatgaaccacctgcaggagacctccctgaaccactgcaagagcaccggcatcctgctggacggctt
cggccgcaccctggagatgtgcaagcgcgacctgatctgggtggtgatcaagatgcagatcaaggtgaaccgctaccccgcctggggcgaca
ccgtggagatcaacacccgcttcagccgcctgggcaagatcggcatgggccgcgactggctgatctccgactgcaacaccggcgagatcctgg
tgcgcgccaccagcgcctacgccatgatgaaccagaagacccgccgcctgtccaagctgccctacgaggtgcaccaggagatcgtgcccctgt
tcgtggacagccccgtgatcgaggactccgacctgaaggtgcacaagttcaaggtgaagaccggcgacagcatccagaagggcctgaccccc
ggctggaacgacctggacgtgaaccagcacgtgtccaacgtgaagtacatcggctggatcctggagagcatgcccaccgaggtgctggagac
ccaggagctgtgctccctggccctggagtaccgccgcgagtgcggccgcgactccgtgctggagagcgtgaccgccatggaccccagcaaggt
gggcgtgcgctcccagtaccagcacctgctgcgcctggaggacggcaccgccatcgtgaacggcgccaccgagtggcgccccaagaacgccg
gcgccaacggcgccatctccaccggcaagaccagcaacggcaactccgtgtccatggactacaaggaccacgacggcgactacaaggacca
cgacatcgactacaaggacgacgacgacaagtgactcgaggcagcagcagctcggatagtatcgacacactctggacgctggtcgtgtgatg
gactgttgccgccacacttgctgccttgacctgtgaatatccctgccgcttttatcaaacagcctcagtgtgtttgatcttgtgtgtacgcgcttttg
cgagttgctagctgcttgtgctatttgcgaataccacccccagcatccccttccctcgtttcatatcgcttgcatcccaaccgcaacttatttacgc
tgtcctgctatccctcagcgctgctcctgctcctgctcactgccctcgcacagccttggtttgggctccgcctgtattctcctggtactgcaacctg
taaaccagcactgcaatgctgatgcacgggaagtagtgggatgggaacacaaatggaaagcttgagctccagcgccatgccacgccctttga
tggcttcaagtacgattacggtgttggattgtgtgtttgttgcgtagtgtgcatggtttagaataatacacttgatttcttgctcacggcaatctcg
gcttgtccgcaggttcaaccccatttcggagtctcaggtcagccgcgcaatgaccagccgctacttcaaggacttgcacgacaacgccgaggtg
agctatgtttaggacttgattggaaattgtcgtcgacgcatattcgcgctccgcgacagcacccaagcaaaatgtcaagtgcgttccgatttgcg
tccgcaggtcgatgttgtgatcgtcggcgccggatccgccggtctgtcctgcgcttacgagctgaccaagcaccctgacgtccgggtacgcgag
ctgagattcgattagacataaattgaagattaaacccgtagaaaaatttgatggtcgcgaaactgtgctcgattgcaagaaattgatcgtcctc
cactccgcaggtcgccatcatcgagcagggcgttgctcccggcggcggcgcctggctggggggacagctgttctcggccatgtgtgtacgtaga
aggatgaatttcagctggttttcgttgcacagctgtttgtgcatgatttgtttcagactattgttgaatgtttttagatttcttaggatgcatgattt
gtctgcatgcgactgaagagcgttt
pSZ5078
SEQ ID NO: 198
agcggaagagcgcccaatgtttaaacccctcaactgcgacgctgggaaccttctccgggcaggcgatgtgcgtgggtttgcctccttggcacgg
ctctacaccgtcgagtacgccatgaggcggtgatggctgtgtcggttgccacttcgtccagagacggcaagtcgtccatcctctgcgtgtgtggc
gcgacgctgcagcagtccctctgcagcagatgagcgtgactttggccatttcacgcactcgagtgtacacaatccatttttcttaaagcaaatga
ctgctgattgaccagatactgtaacgctgatttcgctccagatcgcacagatagcgaccatgttgctgcgtctgaaaatctggattccgaattcg
accctggcgctccatccatgcaacagatggcgacacttgttacaattcctgtcacccatcggcatggagcaggtccacttagattcccgatcacc
cacgcacatctcgctaatagtcattcgttcgtgtcttcgatcaatctcaagtgagtgtgcatggatcttggttgacgatgcggtatgggtttgcgc
cgctggctgcagggtctgcccaaggcaagctaacccagctcctctccccgacaatactctcgcaggcaaagccggtcacttgccttccagattg
ccaataaactcaattatggcctctgtcatgccatccatgggtctgatgaatggtcacgctcgtgtcctgaccgttccccagcctctggcgtcccct
gccccgcccaccagcccacgccgcgcggcagtcgctgccaaggctgtctcggaggtaccctttcttgcgctatgacacttccagcaaaaggtag
ggcgggctgcgagacggcttcccggcgctgcatgcaacaccgatgatgcttcgaccccccgaagctccttcggggctgcatgggcgctccgatg
ccgctccagggcgagcgctgtttaaatagccaggcccccgattgcaaagacattatagcgagctaccaaagccatattcaaacacctagatca
ctaccacttctacacaggccactcgagcttgtgatcgcactccgctaagggggcgcctcttcctcttcgtttcagtcacaacccgcaaactctaga
atatcaatgctgctgcaggccttcctgttcctgctggccggcttcgccgccaagatcagcgcctccatgacgaacgagacgtccgaccgccccct
ggtgcacttcacccccaacaagggctggatgaacgaccccaacggcctgtggtacgacgagaaggacgccaagtggcacctgtacttccagt
acaacccgaacgacaccgtctgggggacgcccttgttctggggccacgccacgtccgacgacctgaccaactgggaggaccagcccatcgcc
atcgccccgaagcgcaacgactccggcgccttctccggctccatggtggtggactacaacaacacctccggcttcttcaacgacaccatcgacc
cgcgccagcgctgcgtggccatctggacctacaacaccccggagtccgaggagcagtacatctcctacagcctggacggcggctacaccttca
ccgagtaccagaagaaccccgtgctggccgccaactccacccagttccgcgacccgaaggtcttctggtacgagccctcccagaagtggatca
tgaccgcggccaagtcccaggactacaagatcgagatctactcctccgacgacctgaagtcctggaagctggagtccgcgttcgccaacgagg
gcttcctcggctaccagtacgagtgccccggcctgatcgaggtccccaccgagcaggaccccagcaagtcctactgggtgatgttcatctccatc
aaccccggcgccccggccggcggctccttcaaccagtacttcgtcggcagcttcaacggcacccacttcgaggccttcgacaaccagtcccgcg
tggtggacttcggcaaggactactacgccctgcagaccttcttcaacaccgacccgacctacgggagcgccctgggcatcgcgtgggcctccaa
ctgggagtactccgccttcgtgcccaccaacccctggcgctcctccatgtccctcgtgcgcaagttctccctcaacaccgagtaccaggccaacc
cggagacggagctgatcaacctgaaggccgagccgatcctgaacatcagcaacgccggcccctggagccggttcgccaccaacaccacgttg
acgaaggccaacagctacaacgtcgacctgtccaacagcaccggcaccctggagttcgagctggtgtacgccgtcaacaccacccagacgat
ctccaagtccgtgttcgcggacctctccctctggttcaagggcctggaggaccccgaggagtacctccgcatgggcttcgaggtgtccgcgtcct
ccttcttcctggaccgcgggaacagcaaggtgaagttcgtgaaggagaacccctacttcaccaaccgcatgagcgtgaacaaccagcccttca
agagcgagaacgacctgtcctactacaaggtgtacggcttgctggaccagaacatcctggagctgtacttcaacgacggcgacgtcgtgtcca
ccaacacctacttcatgaccaccgggaacgccctgggctccgtgaacatgacgacgggggtggacaacctgttctacatcgacaagttccagg
tgcgcgaggtcaagtgacaattgacgcccgcgcggcgcacctgacctgttctctcgagggcgcctgttctgccttgcgaaacaagcccctggag
catgcgtgcatgatcgtctctggcgccccgccgcgcggtttgtcgccctcgcgggcgccgcggccgcgggggcgcattgaaattgttgcaaacc
ccacctgacagattgagggcccaggcaggaaggcgttgagatggaggtacaggagtcaagtaactgaaagtttttatgataactaacaaca
aagggtcgtttctggccagcgaatgacaagaacaagattccacatttccgtgtagaggcttgccatcgaatgtgagcgggcgggccgcggacc
cgacaaaacccttacgacgtggtaagaaaaacgtggcgggcactgtccctgtagcctgaagaccagcaggagacgatcggaagcatcacag
cacaggatcccgcgtctcgaacagagcgcgcagaggaacgctgaaggtctcgcctctgtcgcacctcagcgcggcatacaccacaataacca
cctgacgaatgcgcttggttcttcgtccattagcgaagcgtccggttcacacacgtgccacgttggcgaggtggcaggtgacaatgatcggtgg
agctgatggtcgaaacgttcacagcctagggatatcgaattcggccgacaggacgcgcgtcaaaggtgctggtcgtgtatgccctggccggca
ggtcgttgctgctgctggttagtgattccgcaaccctgattttggcgtcttattttggcgtggcaaacgctggcgcccgcgagccgggccggcggc
gatgcggtgccccacggctgccggaatccaagggaggcaagagcgcccgggtcagttgaagggctttacgcgcaaggtacagccgctcctgc
aaggctgcgtggtggaattggacgtgcaggtcctgctgaagttcctccaccgcctcaccagcggacaaagcaccggtgtatcaggtccgtgtca
tccactctaaagagctcgactacgacctactgatggccctagattcttcatcaaaaacgcctgagacacttgcccaggattgaaactccctgaa
gggaccaccaggggccctgagttgttccttccccccgtggcgagctgccagccaggctgtacctgtgatcgaggctggcgggaaaataggcttc
gtgtgctcaggtcatgggaggtgcaggacagctcatgaaacgccaacaatcgcacaattcatgtcaagctaatcagctatttcctcttcacgag
ctgtaattgtcccaaaattctggtctaccgggggtgatccttcgtgtacgggcccttccctcaaccctaggtatgcgcgcatgcggtcgccgcgca
a ctcgcgcgagggccgagggtttgggacgggccgtcccgaaatgcagttgcacccggatgcgtggcacctiftttgcgataatttatgcaatgg
actgctctgcaaaattctggctctgtcgccaaccctaggatcagcggcgtaggatttcgtaatcattcgtcctgatggggagctaccgactaccc
taatatcagcccgactgcctgacgccagcgtccacttttgtgcacacattccattcgtgcccaagacatttcattgtggtgcgaagcgtccccagt
tacgctcacctgtttcccgacctccttactgttctgtcgacagagcgggcccacaggccggtcgcagccactagtatggccaccgcctccaccttc
tccgccttcaacgcccgctgcggcgacctgcgccgctccgccggctccggcccccgccgccccgcccgccccctgcccgtgcgcgccgccatcaa
ctcccgcgcccaccccaaggccaacggctccgccgtgtccctgaagtccggctccctgaacacccaggaggacacctcctcctcccccccccccc
gcaccttcctgcaccagctgcccgactggtcccgcctgctgaccgccatcaccaccgtgttcgtgaagtccaagcgccccgacatgcacgaccg
caagtccaagcgccccgacatgctgatggactccttcggcctggagtccatcgtgcaggagggcctggagttccgccagtccttctccatccgct
cctacgagatcggcaccgaccgcaccgcctccatcgagaccctgatgaactacctgcaggagacctccctgaaccactgcaagtccaccggca
tcctgctggacggcttcggccgcacccccgagatgtgcaagcgcgacctgatctgggtggtgaccaagatgaagatcaaggtgaaccgctacc
ccgcctggggcgacaccgtggagatcaacacctggttctcccgcctgggcaagatcggcaagggccgcgactggctgatctccgactgcaaca
ccggcgagatcctgatccgcgccacctccgcctacgccaccatgaaccagaagacccgccgcctgtccaagctgccctacgaggtgcaccagg
agatcgcccccctgttcgtggactccccccccgtgatcgaggacaacgacctgaagctgcacaagttcgaggtgaagaccggcgactccatcc
acaagggcctgacccccggctggaacgacctggacgtgaaccagcacgtgtccaacgtgaagtacatcggctggatcctggagtccatgccc
accgaggtgctggagacccaggagctgtgctccctggccctggagtaccgccgcgagtgcggccgcgactccgtgctggagtccgtgaccgcc
atggaccccaccaaggtgggcggccgctcccagtaccagcacctgctgcgcctggaggacggcaccgacatcgtgaagtgccgcaccgagtg
gcgccccaagaaccccggcgccaacggcgccatctccaccggcaagacctccaacggcaactccgtgtccatggactacaaggaccacgacg
gcgactacaaggaccacgacatcgactacaaggacgacgacgacaagtgattaattaactcgaggcagcagcagctcggatagtatcgaca
cactctggacgctggtcgtgtgatggactgttgccgccacacttgctgccttgacctgtgaatatccctgccgcttttatcaaacagcctcagtgt
gtttgatcttgtgtgtacgcgcttttgcgagttgctagctgcttgtgctatttgcgaataccacccccagcatccccttccctcgtttcatatcgcttg
catcccaaccgcaacttatctacgctgtcctgctatccctcagcgctgctcctgctcctgctcactgcccctcgcacagccttggtttgggctccgc
ctgtattctcctggtactgcaacctgtaaaccagcactgcaatgctgatgcacgggaagtagtgggatgggaacacaaatggaaagcttgagc
tccagcgccatgccacgccctttgatggcttcaagtacgattacggtgttggattgtgtgtttgttgcgtagtgtgcatggtttagaataatacac
ttgatttcttgctcacggcaatctcggcttgtccgcaggttcaaccccatttcggagtctcaggtcagccgcgcaatgaccagccgctacttcaag
gacttgcacgacaacgccgaggtgagctatgtttaggacttgattggaaattgtcgtcgacgcatattcgcgctccgcgacagcacccaagca
aaatgtcaagtgcgttccgatttgcgtccgcaggtcgatgttgtgatcgtcggcgccggatccgccggtctgtcctgcgcttacgagctgaccaa
gcaccctgacgtccgggtacgcgagctgagattcgattagacataaattgaagattaaacccgtagaaaaatttgatggtcgcgaaactgtgc
tcgattgcaagaaattgatcgtcctccactccgcaggtcgccatcatcgagcagggcgttgctcccggcggcggcgcctggctggggggacagc
tgttctcggccatgtgtgtacgtagaaggatgaatttcagctggttttcgttgcacagctgtttgtgcatgatttgtttcagactattgttgaatgtt
tttagatttcttaggatgcatgatttgtctgcatgcgactgaagcgtttaaaccgcct

What is claimed:

1. A microalgal cell having an ablated or downregulated fatty acyl-ACP thioesterase (FATA) gene, wherein the cell is modified to express a heterologous lysophosphatidic acid acyltransferase (LPAAT) comprising an amino acid sequence that has at least 80% identity to an acyltransferase encoded by SEQ ID NO: 90, 89, 92, 93 or 95 and wherein the modified microalgal cell produces an oil with an elevated ratio of saturated-unsaturated-saturated triglycerides over trisaturated triglycerides as compared to a corresponding unmodified cell.

2. The microalgal cell of claim 1, wherein the cell is modified to coexpress with the heterologous LPAAT at least one exogenous gene that encodes an enzyme selected from the group consisting of invertase, a fatty acyl-ACP thioesterase, a melibiase, a ketoacyl synthase and a THIC.

3. The microalgal cell of claim 1, wherein the cell is modified to ablate or downregulate the expression of at least one endogenous gene selected from the group consisting of: a stearoyl ACP desaturase, a fatty acyl desaturase, a fatty acyl-ACP thioesterase (FATA or FATB), a ketoacyl synthase (KASI, KASII, KASIII or KAS IV) and an acyltransferase (DGAT, GPAT or LPCAT).

4. The microalgal cell of claim 2, wherein the cell is further modified to overexpress a gene encoding a C18:0-specific FATA1 thioesterase.

5. The microalgal cell of claim 4, wherein the C18:0-specific FATA1 thioesterase is a variant Garcinia thioesterase.

6. The microalgal cell of claim 5, wherein the variant Garcinia thioesterase has at least 80% identity to SEQ ID NO: 142.

7. The microalgal cell of claim 6, wherein the variant Garcinia thioesterase comprises one or more of amino acid variants selected from the group consisting of L91F, L91K, L91S, G96A, G96T, G96V, G108A, G108V, S111A, S111V T156F, T156A, T156K, T156V and V193A.

8. The microalgal cell of claim 7, wherein the variant Garcinia thioesterase is a variant comprising the substitutions S111A and V193A, a variant comprising the substitution G96A, or a variant comprising the substitution G108A.

9. The microalgal cell of claim 2, wherein the ketoacyl synthase is a KASII.

10. The microalgal cell of claim 4, wherein the ketoacyl synthase is a KASII.

11. The microalgal cell of claim 3, wherein the cell is modified to ablate or downregulate the expression of an endogenous stearoyl ACP desaturase-2 (SAD2) gene and an endogenous fatty acyl desaturase-2 (FAD2) gene.

12. The microalgal cell of claim 10, wherein the cell is modified to ablate or downregulate the expression of an endogenous stearoyl ACP desaturase-2 (SAD2) gene and an endogenous fatty acyl desaturase-2 (FAD2) gene.

13. The microalgal cell of claim 1, wherein the cell is modified to express a Theobroma cacao diacylglycerol O-acyltransferase.

14. The microalgal cell of claim 13, wherein the Theobroma cacao diacylglycerol O-acyltransferase is a Theobroma cacao diacylglycerol O-acyltransferase-1 or a Theobroma cacao diacylglycerol O-acyltransferase-2.

15. The microalgal cell of claim 12, wherein the Theobroma cacao diacylglycerol O-acyltransferase is a Theobroma cacao diacylglycerol O-acyltransferase-1 or a Theobroma cacao diacylglycerol O-acyltransferase-2.

16. The microalgal cell of claim 1, wherein the cell is of the genus Prototheca or Chlorella.

17. The microalgal cell of claim 16, wherein the cell is a Prototheca moriformis cell.

18. The microalgal cell of claim 15, wherein the cell is a Prototheca moriformis cell.

19. A method of producing an oil comprising: (a) cultivating the microalgal cell of claim 1 under conditions to produce the oil; and (b) extracting the oil from the microalgal cell; wherein the oil comprises at least 50% stearate-oleate-stearate (SOS) triglycerides with an elevated ratio of saturated-unsaturated-saturated triglycerides over trisaturated triglycerides as compared to a corresponding unmodified cell.

20. A method of producing an oil comprising: (a) cultivating the microalgal cell of claim under conditions to produce the oil; and (b) extracting the oil from the microalgal cell; wherein the oil comprises at least 60% stearate-oleate-stearate (SOS) triglycerides, less than 5% trisaturates and wherein the fatty acid profile of the oil comprises at least 50% C18:0.


SEQUENCE LISTING (198 sequences)

SEQ ID NO: 1 - DNA, 305 bp, Artificial Sequence
  Description of Artificial Sequence Synthetic polynucleotide
gcgaggggtctgcctgggccagccgctccctctgaacacgggacgcgtggtccaattcgggcttcgggaccctttggcggtttgaacgcctgggagagggcgcccgcgagcctggggaccccggcaacggcttccccagagcctgccttgcaatctcgcgcgtcctctccctcagcacgtggcggttccacgtgtggtcgggcgtcccggactagctcacgtcgtgacctagcttaatgaacccagccgggcctgcagcaccaccttagaggttttgattatttgattagaccaatctattcacc

SEQ ID NO: 2 - DNA, 305 bp, Artificial Sequence
  Description of Artificial Sequence Synthetic polynucleotide
ggcgaatagattggtataatgaaataatcaaaacctcttaggcggtgctacaggcccggctgggttcattaagctaggtcacgacgcgagctagtccgggaagcccgaccacacgtggaaccgccacgtgctgagggagaggacgcgcgagattgcaaggcaggctctggggaagccgttgccggggtccccaggctcgcgggcgccccatccctggcgttcaaaccgccaaagggtcccgaagcccgaattggaccacgcgtcccgtgtttagagggagcggctggcccaggcagacccctcgc

SEQ ID NO: 3 - DNA, 305 bp, Artificial Sequence
  Description of Artificial Sequence Synthetic polynucleotide
ggtgaatagattggtctaatcaaataatcaaaacctctaaggtggtgctgcaggcccggctgggttcattaagctaggtcacgacgtgagctagtccgggacgcccgaccacacgtggaaccgccacgtgctgagggagaggacgcgcgagattgcaaggcaggctctggggaagccgttgccggggtccccaggctcgcgggcgccctctcccaggcgttcaaaccgccaaagggtcccgaagcccgaattggaccacgcgtcccgtgttcagagggagcggctggcccaggcagacccctcgc

SEQ ID NO: 4 - DNA, 1322 bp, Artificial Sequence
  Description of Artificial Sequence Synthetic polynucleotide
gtgatgggttctttagacgatccagcccaggatcatgtgttgcccacatggagcctatccacgctggcctagaaggcaagcacatttcaaggtgaacccacgtccatggagcgatggcgccaatatctcgcctctagaccaagcggttctcaccccaactgcgtcatttgtatgtatggctgcaaagttgtcggtacgatagaggccgccaacctggcggcgagggcgaggagctggttgccgatctgtgcccaagcatgtgtcggagctcggctgtctcggcagcgagctcctgtgcaaggggcttgcatcgagaatgtcaggcgatagacactgcacgttggggacacggaggtgcccctgtggcgtgtcctggatgccctcgggtccgtcgcgagaagctctggcgaccagcacccggccacaaccgcagcaggcgttcacccacaagaatcttccagatcgtgatgcgcatgtatcgtgacacgattggcgaggtccgcaggacgcacacggactcgtccactcatcagaactggtcagggcacccatctgcgtcccttttcaggaaccacccaccgctgccaggcaccttcgccagcggcggactccacacagagaatgccttgctgtgagagaccatggccggcaagtgctgtcggatctgcccgcatacggtcagtccccagcacaaggaagccaagagtacaggctgttggtgtcgatggaggagtggccgttcccacaagtagtgagcggcagctgctcaacggcttccccctgttcatcttggcaaagccagtgacttcctacaagtatgtgatgcagatcggcactgcaatctgtcggcatgcgtacagaacatcggctcgccagggcagcgttgctcgctctggatgagctgcttgggaggaatcatcggcacacgcccgtgccgtgcccgcgccccgcgcccgtcgggaaaggcccccggttaggacactgccgcgtcagccagtcgtgggatcgatcggacgtggcgaatcctcgcccggacaccctcatcacaccccacatttccctgcaagcaatcttgccgacaaaatagtcaagatccattgggtttagggaacacgtgcgagactgggcagctgtatctgtccttgccccgcgtcaaattcctgggcgtgacgcagtcacaggagaatctattagaccctggacttgcagctcagtcatgggcgtgagtggctaaagcacctaggtcaggcgagtaccgccccttccccaggattcactcttctgcgattgacgttgagcctgcatcgggctgcttcgtcacc

SEQ ID NO: 5 - DNA, 841 bp, Artificial Sequence
  Description of Artificial Sequence Synthetic polynucleotide
tcggagctaaagcagagactggacaagacttgcgttcgcatactggtgacacagaatagctcccatctattcatacgcctttgggaaaaggaacgagccttgtggcctctgcattgctgcctgctttgaggccgaggacggtgcgggacgctcagatccatcagcgatcgccccaccctcagagcacctccgatccaaggcaatactatcaggcaaagtttccaaattcaaacattccaaaatcacgccagggactggatcacacacgcagatcagcgccgttttgctctttgcctacgggcgactgtgccacttgtcgacccctggtgacgggagggaccacgcctgcggttggcatccacttcgacggacccagggacggtttctcatgccaaacctgagatttgagcacccagatgagcacattatgcgttttaggatgcctgagcagcgggcgtgcaggaatctggtctcgccagattcaccgaagatgcgcccatcggagcgaggcgagggctttgtgaccacgcaaggcagtgtgaggcaaacacatagggacacctgcgtctttcaatgcacagacatctatggtgcccatgtatataaaatgggctacttctgagtcaaaccaacgcaaactgcgctatggcaaggccggccaaggttggaatcccggtctgtctggatttgagtttgtgggggctatcacgtgacaatccctgggattgggcggcagcagcgcacggcctgggtggcaatggcgcactaatactgctgaaagcacggctctgcatccctttctcttgacctgcgattggtccttttcgcaagcgtgatcatc

SEQ ID NO: 6 - DNA, 841 bp, Artificial Sequence
  Description of Artificial Sequence Synthetic polynucleotide
tcggagctaaagcagaaactgaacaagacttgcgttcgcatacttgtgacactgaataggttcaatctattcatacgcctttgggaaactgaacgagccttgtggcctctgcattgctgcctgctttgaggccgaggacggcgcggaacgcacagatccatcagcgatcgccccaccctcagagtacatccgatccaaggcaatactatcaggcaaagtttccaaattcaaacattccaaaattacgtcagggactggatcacacacgcagatcagcgccgttttgctctttgcctacgggcgactgtgccacttgtcgacgcctggtgacgggagggaccacgcctgcggttggcatccacttcgacggacccagggacggtctcacatgccaaacctgagatttgagcaccaagatgagcacattatgcgtttttggatgcctgagcagcgggcgtgcaggaatctggtctcgccagattcaccgaagatgcggccatcggagcgaggcgagggctgtgtggccacgccaggcagtgtgaggcaaacacacagggacatctgcttctttcgatgcacagacatctatgttgcccgtgcatataaaatgggctacttctgaatcaaaccaacgcaaacttcgctatggcaaggccggccaaggttggaatcccggtctgtctggatttgagtttgtgggggctatcacgtgacaatccctgggattgggcggcagcagcgcacggcctggatggcaatggcgcactaatactgctgaaagcacggctctgcatccctttctcttgacctgcgattggtccttttcgcaagcgtgatcatc

SEQ ID NO: 7 - DNA, 512 bp, Artificial Sequence
  Description of Artificial Sequence Synthetic polynucleotide
caccgatcactccgtcgccgcccaagagaaatcaacctcgatggagggcgaggtggatcagaggtattggttatcgttcgttcttagtctcaatcaatcgtacaccttgcagttgcccgagtttctccacacatacagcacctcccgctcccagcccattcgagcgacccaatccgggcgatcccagcgatcgtcgtcgcttcagtgctgaccggtggaaagcaggagatctcgggcgagcaggaccacatccagcccaggatcttcgactggctcagagctgaccctcacgcggcacagcaaaagtagcacgcacgcgttatgcaaactggttacaacctgtccaacagtgttgcgacgttgactggctacattgtctgtctgtcgcgagtgcgcctgggcccttacggtgggacactggaactccgccccgagtcgaacacctagggcgacgcccgcagcttggcatgacagctctccttgtgttctaaataccttgcgcgtgtgggaga

SEQ ID NO: 8 - DNA, 516 bp, Artificial Sequence
  Description of Artificial Sequence Synthetic polynucleotide
atccaccgatcactccgtcgccgcccaagagaattcaacctcgatggagggcaaggtggatcagaggtattggttatcgttcgctattagtctcaatcaatcgtgcaccttgcagttgctcgagtttctccacacatacagcacctcccgctcccagcccattcgagcgacccaatccgggcgatcccagcgatcgtcgtcgcttcagtgctgaccggtggaaagcaggagatctcgggcgagcaggaccacatccagcacaggatcttcgactggctcagagctgaccctcacgcggcacagcaaaagtagcccgcacgcgttatgcaaacaggttacaacctgtccaacactgttgcgacgttgactggctacattgtctgtctgtcgcgagtacgcctggacccttacggtgggacactggaactccgccccgagtcgaacacctagggcgacgcccgcagcttggcatgacagctctccttgtattctaaatacctcgcgcgtgtgggagaa

SEQ ID NO: 9 - DNA, 335 bp, Artificial Sequence
  Description of Artificial Sequence Synthetic polynucleotide
atgatgcgcgtgtacgactatcaaggaagaaagaggacttaatttcttaccttctaaccaccatattctttttgctggatgcttgctcgtctcgatgacaattgtgaacctcttgtgtgaccctgaccctgctgcaaggctctccgaccgcacgcaaggcgcagccggcgcgtccggaggcgatcggatccaatccagtcgtcctcccgcagcccgggcacgtttgcccatgcaggcccttccacaccgctcaagagactcccgaacaccgcccactcggcactcgcttcggctgccgagtgcgcgtttgagtttgccctgccacagaagacacc

SEQ ID NO: 10 - DNA, 335 bp, Artificial Sequence
  Description of Artificial Sequence Synthetic polynucleotide
atgatgcgcgtgtacgactatcaaggaagaaagaggacttaatttcttaccttctaaccaccatattctttttgctggatgcttgctcgtctcgatgacaattgtgaacctcttgtgtgaccctgaccctgctgcaaggctctccgaccgcacgcaaggcgcagccggcgcgtccggaggcgatcggatccaatccagtcgtcctcccgcagcccgggcacgtttgcccatgcaggcccttccacaccgctcaagagactcccgaacaccgcccactcggcactcgcttcggctgccgagtgcgcgtttgagtttgccctgccacaggagacatc

SEQ ID NO: 11 - DNA, 1097 bp, Artificial Sequence
  Description of Artificial Sequence Synthetic polynucleotide
cccgggcgagctgtacgcctacggagcgaggcctggtgtgaccgttgcgatctcgccagcagacgtcgcggagcctcgtcccaaaggccctttctgatcgagcttgtcgtccactggacgctttaagttgcgcgcgcgatgggataaccgagctgatctgcactcagattttggtttgttttcgcgcatggtgcagcgaggggaggtactacgctggggtacgagatcctccggattcccagaccgtgttgccggcatttacccggtcatcgccagcgattcgggacgacaaggccttatcctgtgctgagacgctcgagcacgtttataaaattgtgggtaccgcggtatgcacagcgttcaacacgcgccacgccgaaattggttggtgggggagcacgtatgggactgacgtatggccagcagcgaacactcaccgaacaagtgccaatgtataccttgcatcaatgatgctccggcagcttcgattgactgtctcgaaaaagtgtgagcaagcagatcatgtggccgctctgtcgcgcagcacctgacgcattcgacacccacggcaatgcccaggccagggaatagagagtaagacaactcccattgttcagcaaaacattgcactgcagtgccttcacaactatacaatgaatgggagggaatatgggctctgcatgggacagcttagctgggacattcggctactgaacaagaaaaccccacgagaaccaattggcgaaacctgccgggaggaggtgatcgtttctgtaaatggcttacgcattcccccccggcggctcacgaggggtgtggtgaaccctgccagctgatcaagtgcttgctgacgtcggccagggaggtgtatgtgattgggccgtggggcgtgagttatcctaccgccggacccgcgaagtcacatgacgaatggccgtgcgggatgacgagagcacgactcgctctttcttcgccggcccggcttcatggaggacaataataaagggtggccaccggcaacagccctccatacctgaaccgattccagacccaaacctcttgaattttgagggatccagttcaccggtatagtcacg

SEQ ID NO: 12 - DNA, 1105 bp, Artificial Sequence
  Description of Artificial Sequence Synthetic polynucleotide
atccccgggcgagctgtacgcctacggagcgaggcctggtgtgaccgttgcgatctcgccagcagacgtcgcggagcctcgtcccaaaggccctttctgatcgagcttgtcgtccactggacgctttaagttgcgcgcgcgatgggataaccgagctgatctgcactcagattttggtttgttttcgcgcatggtgcagcgaggggaggtactacgctggggtacgagatcctccggattcccagaccgtgttgccggcatttacccggtcatcgccagcgattcgggacgacaaggccttatcctgtgctgagacgctcgagcacgtttataaaattgtggtcaccgtggtacgcacagcgtccaacacgcgccacgccgaaattcgttggtgggggagcacgtatcggactgacgtatggccagcagcgaacactcaccaaacaggtgccaatgtatagcttgcatcaatgatgctctggcagcttcgattgactgtctcgaaaaagtgtgtgcaaacagattatgtggccgctctgtggccgcgcagcacctgacgcactcgacacccacggcaatgcccaggccaaggaacagagagtaagacaactcccattgttcagtaaaacattgcactgcagtgccttcacaaacatacaacgaatgggagggaatatgggcttcgaatgggacagcttagctgggacattcggttactgaacaagaaaaccccacgagaaccaactggcgaaacctgccgggaggaggtgatcgtttttgtaaatggcttacgcattccccccccggcggctcacggggggtgtggtgaaccctgccagctgatcaagtgcttgctgacgtcggccagggaggtgtatgtgatttggccgtggggcgtgagttatcctaccgccggacccgcgaagtcacatgacgaatggccgtgcgggatgacgagagcagggctcgctctttcttcgccggcccggcttcatggaggacaataataaagggtggccaccggcaacagccctccatacctgaaccgattccagacccaaacctcttgaattttgagggatccagttcaccggtatagtcacga

SEQ ID NO: 13 - DNA, 754 bp, Artificial Sequence
  Description of Artificial Sequence Synthetic polynucleotide
gcgagtggttttgctgccgggaagggagtggggagcgtcgagcgagggacgcggcgctcgaggcgcacgtcgtctgtcaacgcgcgcggccctcgcggcccgcggccccacccagctctaatcatcgaaaactaagaggctccacacgcctgtcgtagaatgcatgggattcgccagtagaccacgatctgcgccgaagaagctggtctacccgacgttttttgttgctcctttattctgaatgatatgaagatagtgtgcgcagtgccacgcataggcatcaggagcaagggaggacgggtcaacttgaaagaaccaaaccatccatccgagaaatgcgcatcatctttgtagtaccatcaaacgccttggccaatgtcttctgcatggacaacacaacctgctcctggccacacggtcgacttggagcgccccatgcgcccaggtcgccacgacccgcggcccagcgcgcggcgattcgcctcacgagatcccggcggacccggcacgcccgcgggccgacggtgcgcttggcgatgctgctcattaacccacggccgtcacccgatccacatgctctttttcaacacatccacattggaatagagctctaccagggtgagtactgcattctttggggctgggaggaccccactcgacacctggtccttcatcggccgaaagcccgaacctgagcgcttccccgccccgttcctcatccccgactttccgatggcccattgcagtttcaaac

SEQ ID NO: 14 - DNA, 318 bp, Artificial Sequence
  Description of Artificial Sequence Synthetic polynucleotide
atctgggtggaggactgggagtaagatgtaaggatattaattaaacattctagtttgttgatggcacaacagtcaatgcatttcagtcgtcttgctccttataacctatgcgtgtgccatcgccggccatgcacctgtggcgtggtaccgaccatcggggagaggcccgagattcggaggtacctcccgccctgggcgagcccttcacgtgacggcacaagtcccttgcatcggcccgcgagcacggaatacagagccccgtgccccccacgggccctcacatcatccactccattgttcttgccacaccgatcagca

SEQ ID NO: 15 - DNA, 316 bp, Artificial Sequence
  Description of Artificial Sequence Synthetic polynucleotide
tgggtggaggactgggaagaagatgtaaggatatcaatttaacattctagtttgttgatggcacaacagtcactgaataccgggcgtctggctgctaaaatagccggagcgtgtgccatcgccggccatgcatctgtggcgtggtaccgaccatcagggagaggcccgagattcggaggtacctcccgccctgggcgagcccttcacgtgacggcacaagtcccttgcatcggcccgcgagcacggaatacagagccccgtgctccccacgggccctcacatcatccactccattgttcttgccacaccgatcagc

SEQ ID NO: 16 - DNA, 350 bp, Artificial Sequence
  Description of Artificial Sequence Synthetic polynucleotide
ataacgaggcacaatgatcgatatttctatcgaacaactgtatttagccctgtacgtaccccgctcttgggccagcccgtccgtgcttgccttcggaaaattgcatggcgcctcatgcaaactcgcgctctcacagcagatctcgcccagctcccgggagagcaatcgcgggtggggcccggggcgaatccaggacgcgccccgcggggccgctccactcgccagggccaatgggcggcttatagtcctggcatgggctctgcatgcacagtatcgcagtttgggcgaggtgttgcccccgcgatttcgaatacgcgacgcccggtactcgtgcgagaacagggttcttg

SEQ ID NO: 17 - DNA, 772 bp, Prototheca moriformis
tcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcggggctggcgggaaaacaggcttcgtgtgctcaggttatgggaggtgcaggacagctcattaaacgccaacaatcgcacaattcatggcaagctaatcagttatttcccattaacgagctataattgtcccaaaattctggtctaccgggggtgatccttcgtgtacgggcccttccctcaaccctaggtatgcgcacatgcggtcgccgcgcaacgcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggtgtaggatttcgtaatcattcgtcctgatggggagctaccgactgccctagtatcagcccgactgcctgacgccagcgtccacttttgtgcacacattccattcgtgcccaagacatttcattgtggtgcgaagcgtccccagttacgctcacctgatccccaacctccttattgttctgtcgacagagtgggcccagaggccggtcgcagcc

SEQ ID NO: 18 - DNA, 1065 bp, Prototheca moriformis
ggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagcc

SEQ ID NO: 19 - DNA, 6332 bp, Artificial Sequence
  Description of Artificial Sequence Synthetic polynucleotide
gctcttccgctaacggaggtctgtcaccaaatggaccccgtctattgcgggaaaccacggcgatggcacgtttcaaaacttgatgaaatacaatattcagtatgtcgcgggcggcgacggcggggagctgatgtcgcgctgggtattgcttaatcgccagcttcgcccccgtcttggcgcgaggcgtgaacaagccgaccgatgtgcacgagcaaatcctgacactagaagggctgactcgcccggcacggctgaattacacaggcttgcaaaaataccagaatttgcacgcaccgtattcgcggtattttgttggacagtgaatagcgatgcggcaatggcttgtggcgttagaaggtgcgacgaaggtggtgccaccactgtgccagccagtcctggcggctcccagggccccgatcaagagccaggacatccaaactacccacagcatcaacgccccggcctatactcgaaccccacttgcactctgcaatggtatgggaaccacggggcagtcttgtgtgggtcgcgcctatcgcggtcggcgaagaccgggaaggtaccgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatgactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacgtactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagaattcggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccactagtatggccatccccgccgccgccgtgatcttcctgttcggcctgctgttcttcacctccggcctgatcatcaacctgttccaggccctgtgcttcgtgctggtgtggcccctgtccaagaacgcctaccgccgcatcaaccgcgtgttcgccgagctgctgctgtccgagctgctgtgcctgttcgactggtgggccggcgccaagctgaagctgttcaccgaccccgagaccttccgcctgatgggcaaggagcacgccctggtgatcatcaaccacatgaccgagctggactggatgctgggctgggtgatgggccagcacctgggctgcctgggctccatcctgtccgtggccaagaagtccaccaagttcctgcccgtgctgggctggtccatgtggttctccgagtacctgtacatcgagcgctcctgggccaaggaccgcaccaccctgaagtcccacatcgagcgcctgaccgactaccccctgcccttctggatggtgatcttcgtggagggcacccgcttcacccgcaccaagctgctggccgcccagcagtacgccgcctcctccggcctgcccgtgccccgcaacgtgctgatcccccgcaccaagggcttcgtgtcctgcgtgtcccacatgcgctccttcgtgcccgccgtgtacgacgtgaccgtggccttccccaagacctcccccccccccaccctgctgaacctgttcgagggccagtccatcgtgctgcacgtgcacatcaagcgccacgccatgaaggacctgcccgagtccgacgacgccgtggcccagtggtgccgcgacaagttcgtggagaaggacgccctgctggacaagcacaacgccgaggacaccttctccggccaggaggtgcaccgcaccggctcccgccccatcaagtccctgctggtggtgatctcctgggtggtggtgatcaccttcggcgccctgaagttcctgcagtggtcctcctggaagggcaaggccttctccgtgatcggcctgggcatcgtgaccctgctgatgcacatgctgatcctgtcctcccaggccgagcgctcctccaaccccgccaaggtggcccaggccaagctgaagaccgagctgtccatctccaagaaggccaccgacaaggagaactgactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttgagctcagcggcgacggtcctgctaccgtacgacgttgggcacgcccatgaaagtttgtataccgagcttgttgagcgaactgcaagcgcggctcaaggatacttgaactcctggattgatatcggtccaataatggatggaaaatccgaacctcgtgcaagaactgagcaaacctcgttacatggatgcacagtcgccagtccaatgaacattgaagtgagcgaactgttcgcttcggtggcagtactactcaaagaatgagctgctgttaaaaatgcactctcgttctctcaagtgagtggcagatgagtgctcacgccttgcacttcgctgcccgtgtcatgccctgcgccccaaaatttgaaaaaagggatgagattattgggcaatggacgacgtcgtcgctccgggagtcaggaccggcggaaaataagaggcaacacactccgcttcttagctcttc

SEQ ID NO: 20 - DNA, 1188 bp, Artificial Sequence
  Description of Artificial Sequence Synthetic polynucleotide
actagtatggccatcccctccgccgccgtggtgttcctgttcggcctgctgttcttcacctccggcctgatcatcaacctgttccaggccttctgcttcgtgctgatctcccccctgtccaagaacgcctaccgccgcatcaaccgcgtgttcgccgagctgctgcccctggagttcctgtggctgttccactggtgcgccggcgccaagctgaagctgttcaccgaccccgagaccttccgcctgatgggcaaggagcacgccctggtgatcatcaaccacaagatcgagctggactggatggtgggctgggtgctgggccagcacctgggctgcctgggctccatcctgtccgtggccaagaagtccaccaagttcctgcccgtgttcggctggtccctgtggttctccggctacctgttcctggagcgctcctgggccaaggacaagatcaccctgaagtcccacatcgagtccctgaaggactaccccctgcccttctggctgatcatcttcgtggagggcacccgcttcacccgcaccaagctgctggccgcccagcagtacgccgcctcctccggcctgcccgtgccccgcaacgtgctgatcccccacaccaagggcttcgtgtcctccgtgtcccacatgcgctccttcgtgcccgccatctacgacgtgaccgtggccttccccaagacctcccccccccccaccatgctgaagctgttcgagggccagtccgtggagctgcacgtgcacatcaagcgccacgccatgaaggacctgcccgagtccgacgacgccgtggcccagtggtgccgcgacaagttcgtggagaaggacgccctgctggacaagcacaactccgaggacaccttctccggccaggaggtgcaccacgtgggccgccccatcaaggccctgctggtggtgatctcctgggtggtggtgatcatcttcggcgccctgaagttcctgctgtggtcctccctgctgtcctcctggaagggcaaggccttctccgtgatcggcctgggcatcgtggccggcatcgtgaccctgctgatgcacatcctgatcctgtcctcccaggccgagggctccaaccccgtgaaggccgcccccgccaagctgaagaccgagctgtcctcctccaagaaggtgaccaacaaggagaactgactcgag

SEQ ID NO: 21 - DNA, 1122 bp, Artificial Sequence
  Description of Artificial Sequence Synthetic polynucleotide
actagtatggccatcgccgccgccgccgtgatcttcctgttcggcctgctgttcttcgcctccggcatcatcatcaacctgttccaggccctgtgcttcgtgctgatctggcccctgtccaagaacgtgtaccgccgcatcaaccgcgtgttcgccgagctgctgctgatggacctgctgtgcctgttccactggtgggccggcgccaagatcaagctgttcaccgaccccgagaccttccgcctgatgggcatggagcacgccctggtgatcatgaaccacaagaccgacctggactggatggtgggctggatcctgggccagcacctgggctgcctgggctccatcctgtccatcgccaagaagtccaccaagttcatccccgtgctgggctggtccgtgtggttctccgagtacctgttcctggagcgctcctgggccaaggacaagtccaccctgaagtcccacatggagaagctgaaggactaccccctgcccttctggctggtgatcttcgtggagggcacccgcttcacccgcaccaagctgctggccgcccagcagtacgccgcctcctccggcctgcccgtgccccgcaacgtgctgatcccccacaccaagggcttcgtgtcctgcgtgtccaacatgcgctccttcgtgcccgccgtgtacgacgtgaccgtggccttccccaagtcctcccccccccccaccatgctgaagctgttcgagggccagtccatcgtgctgcacgtgcacatcaagcgccacgccctgaaggacctgcccgagtccgacgacgccgtggcccagtggtgccgcgacaagttcgtggagaaggacgccctgctggacaagcacaacgccgaggacaccttctccggccaggaggtgcaccacatcggccgccccatcaagtccctgctggtggtgatcgcctgggtggtggtgatcatcttcggcgccctgaagttcctgcagtggtcctccctgctgtccacctggaagggcaaggccttctccgtgatcggcctgggcatcgccaccctgctgatgcacatgctgatcctgtcctcccaggccgagcgctccaaccccgccaaggtggccaagtgactcgag

SEQ ID NO: 22 - DNA, 1188 bp, Artificial Sequence
  Description of Artificial Sequence Synthetic polynucleotide
actagtatggccatcccctccgccgccgtggtgttcctgttcggcctgctgttcttcacctccggcctgatcatcaacctgttccaggccttctgcttcgtgctgatctcccccctgtccaagaacgcctaccgccgcatcaaccgcgtgttcgccgagctgctgcccctggagttcctgtggctgttccactggtgcgccggcgccaagctgaagctgttcaccgaccccgagaccttccgcctgatgggcaaggagcacgccctggtgatcatcaaccacaagatcgagctggactggatggtgggctgggtgctgggccagcacctgggctgcctgggctccatcctgtccgtggccaagaagtccaccaagttcctgcccgtgttcggctggtccctgtggttctccgagtacctgttcctggagcgctcctgggccaaggacaagatcaccctgaagtcccacatcgagtccctgaaggactaccccctgcccttctggctgatcatcttcgtggagggcacccgcttcacccgcaccaagctgctggccgcccagcagtacgccgcctcctccggcctgcccgtgccccgcaacgtgctgatcccccacaccaagggcttcgtgtcctccgtgtcccacatgcgctccttcgtgcccgccatctacgacgtgaccgtggccttccccaagacctcccccccccccaccatgctgaagctgttcgagggccagtccgtggagctgcacgtgcacatcaagcgccacgccatgaaggacctgcccgagtccgacgacgccgtggcccagtggtgccgcgacaagttcgtggagaaggacgccctgctggacaagcacaactccgaggacaccttctccggccaggaggtgcaccacgtgggccgccccatcaaggccctgctggtggtgatctcctgggtggtggtgatcatcttcggcgccctgaagttcctgctgtggtcctccctgctgtcctcctggaagggcaaggccttctccgtgatcggcctgggcatcgtggccggcatcgtgaccctgctgatgcacatcctgatcctgtcctcccaggccgagggctccaaccccgtgaaggccgcccccgccaagctgaagaccgagctgtcctcctccaagaaggtgaccaacaaggagaactgactcgag

SEQ ID NO: 23 - protein, 385 aa, Artificial Sequence
  Description of Artificial Sequence Synthetic polypeptide
MAIPAAAVIFLFGLLFFTSGLIINLFQALCFVLVWPLSKNAYRRINRVFAELLLSELLCLFDWWAGAKLKLFTDPETFRLMGKEHALVIINHMTELDWMLGWVMGQHLGCLGSILSVAKKSTKFLPVLGWSMWFSEYLYIERSWAKDRTTLKSHIERLTDYPLPFWMVIFVEGTRFTRTKLLAAQQYAASSGLPVPRNVLIPRTKGFVSCVSHMRSFVPAVYDVTVAFPKTSPPPTLLNLFEGQSIVLHVHIKRHAMKDLPESDDAVAQWCRDKFVEKDALLDKHNAEDTFSGQEVHRTGSRPIKSLLVVISWVVVITFGALKFLQWSSWKGKAFSVIGLGIVTLLMHMLILSSQAERSSNPAKVAQAKLKTELSISKKATDKEN

SEQ ID NO: 24 - protein, 384 aa, Artificial Sequence
  Description of Artificial Sequence Synthetic polypeptide
MAIPAAAVIFLFGLIFFASGLIINLFQALCFVLIWPISKNAYRRINRVFAELLLSELLCLFDWWAGAKLKLFTDPETFRLMGKEHALVIINHMTELDWMVGWVMGQHFGCLGSILSVAKKSTKFLPVLGWSMWFTEYLYIERSWNKDKSTLKSHIERLKDYPLPFWLVIFAEGTRFTQTKLLAAQQYAASSGLPVPRNVLIPRTKGFVSCVSHMRSFVPAVYDLTVAFPKTSPPPTLLNLFEGQSVVLHVHIKRHAMKDLPESDDEVAQWCRDKFVEKDALLDKHNAEDTFSGQELQHTGRRPIKSLLVVISWVVVIAFGALKFLQWSSWKGKAFSVIGLGIVTLLMHMLILSSQAERSKPAKVAQAKLKTELSISKTVTDKEN

SEQ ID NO: 25 - protein, 278 aa, Artificial Sequence
  Description of Artificial Sequence Synthetic polypeptide
MAIPAAAVIFLFGLIFFASGLIINLFQALCFVLIWPISKNAYRRINRVFAELLLSELLCLFDWWAGAKLKLFTDPETFRLMGKEHALVIINHMTELDWMVGWVMGQHFGCLGSILSVAKKSTKFLPVLGWSMWFTEYLYIERSWNKDKSTLKSHIERLKDYPLPFWLVIFAEGTRFTQTKLLAAQQYAASSGLPVPRNVLIPRTKGFVSCVSHMRSFVPAVYDLTVAFPKTSPPPTLLNLFEGQSVVLHVHIKRHAMKDLPESDDEVAQWCRDKFVEK

SEQ ID NO: 26 - protein, 366 aa, Artificial Sequence
  Description of Artificial Sequence Synthetic polypeptide
IVNLVQAVCFVLVRPLSKNTYRRINRVVAELLWLELVWLIDWWAGVKIKVFTDHETFHLMGKEHALVICNHKSDIDWLVGWVLAQRSGCLGSTLAVMKKSSKFLPVIGWSMWFSEYLFLERNWAKDESTLKSGLNRLKDYPLPFWLALFVEGTRFTRAKLLAAQQYAASSGLPVPRNVLIPRTKGFVSSVSHMRSFVPAIYDVTVAIPKTSPPPTLIRMFKGQSSVLHVHLKRHVMKDLPESDDAVAQWCRDIFVEKDALLDKHNADDTFSGQELQDTGRPIKSLLVVISWAVLEVFGAVKFLQWSSLLSSWKGLAFSGIGLGIITLLMHILILFSQSERSTPAKVAPAKAKIEGESSKTEMEKEK

SEQ ID NO: 27 - protein, 287 aa, Artificial Sequence
  Description of Artificial Sequence Synthetic polypeptide
IVNLVQAVCFVLVRPLSKNTYRRINRVVAELLWLELVWLIDWWAGVKIKVFTDHETFHLMGKEHALVICNHKSDIDWLVGWVLAQRSGCLGSTLAVMKKSSKFLPVIGWSMWFSEYLFLERNWAKDESTLKSGLNRLKDYPLPFWLALFVEGTRFTRAKLLAAQQYAASSGLPVPRNVLIPRTKGFVSSVSHMRSFVPAIYDVTVAIPKTSPPPTLIRMFKGQSSVLHVHLKRHVMKDLPESDDAVAQWCRDIFVEKDALLDKHNADDTFSGQELQDTGRPIKSLLV

SEQ ID NO: 28 - protein, 391 aa, Artificial Sequence
  Description of Artificial Sequence Synthetic polypeptide
MAIPSAAVVFLFGLLFFTSGLIINLFQAFCFVLISPLSKNAYRRINRVFAELLPLEFLWLFHWCAGAKLKLFTDPETFRLMGKEHALVIINHKIELDWMVGWVLGQHLGCLGSILSVAKKSTKFLPVFGWSLWFSGYLFLERSWAKDKITLKSHIESLKDYPLPFWLIIFVEGTRFTRTKLLAAQQYAASSGLPVPRNVLIPHTKGFVSSVSHMRSFVPAIYDVTVAFPKTSPPPTMLKLFEGQSVELHVHIKRHAMKDLPESDDAVAQWCRDKFVEKDALLDKHNSEDTFSGQEVHHVGRPIKALLVVISWVVVIIFGALKFLLWSSLLSSWKGKAFSVIGLGIVAGIVTLLMHILILSSQAEGSNPVKAAPAKLKTELSSSKKVTNKEN

SEQ ID NO: 29 - protein, 391 aa, Artificial Sequence
  Description of Artificial Sequence Synthetic polypeptide
MAIPSAAVVFLFGLLFFTSGLIINLFQAFCFVLISPLSKNAYRRINRVFAELLPLEFLWLFHWCAGAKLKLFTDPETFRLMGKEHALVIINHKIELDWMVGWVLGQHLGCLGSILSVAKKSTKFLPVFGWSLWFSEYLFLERSWAKDKITLKSHIESLKDYPLPFWLIIFVEGTRFTRTKLLAAQQYAASSGLPVPRNVLIPHTKGFVSSVSHMRSFVPAIYDVTVAFPKTSPPPTMLKLFEGQSVELHVHIKRHAMKDLPESDDAVAQWCRDKFVEKDALLDKHNSEDTFSGQEVHHVGRPIKALLVVISWVVVIIFGALKFLLWSSLLSSWKGKAFSVIGLGIVAGIVTLLMHILILSSQAEGSNPVKAAPAKLKTELSSSKKVTNKEN

SEQ ID NO: 30 - protein, 376 aa, Artificial Sequence
  Description of Artificial Sequence Synthetic polypeptide
LSLLFFVSGLIVNLVQAVCFVLIRPLSKNTYRRINRVVAELLWLELVWLIDWWAGVKIKVFTDHETFNLMGKEHALVVCNHKSDIDWLVGWVLAQRSGCLGSTLAVMKKSSKFLPVIGWSMWFSEYLFLERSWAKDESTLKSGLKRLKDYPLPFWLALFVEGTRFTQAKLLAAQQYAASSGLPVPRNVLIPRTKGFVSSVSHMRSFVPAIYDVTVAIPKTSVPPTMLRIFKGQSSVLHVHLKRHLMKDLPESDDAVAQWCRDIFVEKDALLDKHNAEDTFSGQELQDIGRPIKSLLVVISWAVLVIFGAVKFLQWSSLLSSWKGLAFSGIGLGIVTLLMHILILFSQSERSTPAKVAPAKPKNEGESSKTEMEKEH

SEQ ID NO: 31 - protein, 320 aa, Artificial Sequence
  Description of Artificial Sequence Synthetic polypeptide
QIKVFTDHETFNLMGKEHALVVCNHKSDIDWLVGWVLAQWSGCLGSTLAVMKKSSKFLPVIGWSMWFSEYLFLERSWAKDESTLKSGLKRLKDYPLPFWLALFVEGTRFTQAKLLAAQQYAASSGLPVPRNVLIPRTKGFVSSVSHMRSFVPAIYDVTVAIPKTSVPPTMLRIFKGQSSVLHVHLKRHLMKDLPESDDAVAQWCRDIFVEKDALLDKHNAEDTFSGQELQDIGRPIKSLLVVISWAVLVIFGAVKFLQWSSLLSSWKGLAFSGIGLGIVTLLMHILILFSQSERSTPAKVAPAKLKKEGESSKPETDKQN

SEQ ID NO: 32 - protein, 376 aa, Artificial Sequence
  Description of Artificial Sequence Synthetic polypeptide
LSLLFFVSGLIVNLVQAVCFVLIRPLLKNTYRRINRVVAELLWLELVWLIDWWAGIKIKVFTDHETFHLMGKEHALVICNHKSDIDWLVGWVLAQRSGCLGSTLAVMKKSSKFLPVIGWSMWFSEYLFLERNWAKDESTLKSGLNRLKDYPLPFWLALFVEGTRFTRAKLLAAQQYAASSGLPVPRNVLIPRTKGFVSSVSQMRSFVPAIYDVTVAIPKTSPPPTLLRMFKGQSSVLHVHLKRHLMNDLPESDDAVAQWCRDIFVEKDALLDKHNAEDTFSGQELQDTGRPIKSLLVVISWATLVVFGAVKFLQWSSLLSSWKGLAFSGIGLGIITLLMHILILFSQSERSTPAKVAPAKPKNEGESSKTEMEKEH

SEQ ID NO: 33 - protein, 376 aa, Artificial Sequence
  Description of Artificial Sequence Synthetic polypeptide
LSLLFFVSGLIVNLVQAVCFVLIRPLLKNTYRRINRVVAELLWLELVWLIDWWAGIKIKVFTDHETFHLMGKEHALVICNHKSDIDWLVGWVLAQRSGCLGSTLAVMKKSSKFLPVIGWSMWFSEYLFLERNWAKDESTLKSGLNRLKDYPLPFWLALFVEGTRFTRAKLLAAQQYAASSGLPVPRNVLIPRTKGFVSSVSQMRSFVPAIYDVTVAIPKTSPPPTLLRMFKGQSSVLHVHLKRHLMNDLPESDDAVAQWCRDIFVEKDALLDKHNAEDTFSGQELQDIGRPIKSLLVVISWAVLEIFGAVKFLQWSSLLSSWKGLAFSGIGLGIVTLLMHILILFSQSERSTPAKVAPAKPKKEGESSKPETDKEN

SEQ ID NO: 34 - protein, 369 aa, Artificial Sequence
  Description of Artificial Sequence Synthetic polypeptide
MAIAAAAVIFLFGLLFFASGIIINLFQALCFVLIWPLSKNVYRRINRVFAELLLMDLLCLFHWWAGAKIKLFTDPETFRLMGMEHALVIMNHKTDLDWMVGWILGQHLGCLGSILSIAKKSTKFIPVLGWSVWFSEYLFLERSWAKDKSTLKSHMEKLKDYPLPFWLVIFVEGTRFTRTKLLAAQQYAASSGLPVPRNVLIPHTKGFVSCVSNMRSFVPAVYDVTVAFPKSSPPPTMLKLFEGQSIVLHVHIKRHALKDLPESDDAVAQWCRDKFVEKDALLDKHNAEDTFSGQEVHHIGRPIKSLLVVIAWVVVIIFGALKFLQWSSLLSTWKGKAFSVIGLGIATLLMHMLILSSQAERSNPAKVAK

SEQ ID NO: 35 - protein, 388 aa, Artificial Sequence
  Description of Artificial Sequence Synthetic polypeptide
MAIAAAAVIFLFGLLFFASGIIINLFQALCFVLIWPLSKNVYRRINRVFAELLLMDLLCLFHWWAGAKIKLFTDPETFRLMGMEHALVIMNHKTDLDWMVGWILGQHLGCLGSILSIAKKSTKFIPVLGWSVWFSEYLFLERSWAKDESTLKSGLNRLKDYPLPFWLALFVEGTRFTRAKLLAAQQYAASSGLPVPKNVLIPRTKGFVSSVSHMRSFVPAIYDVTVAIPKTSAPPTLLRMFKGQSSVLHVHLKRHLMKDLPESDDAVAQWCRDIFVEKDALLDKHNAEDTFSGQELHDIGRPVKSLLVVISWAMLVVFGAVKFLQWSSLLSSWKGLAFSGIGLGIITLLMHILILFSQSERSTPAKVAPAKQKNNEGESSKTEMEKEH

SEQ ID NO: 36 - protein, 354 aa, Artificial Sequence
  Description of Artificial Sequence Synthetic polypeptide
SGLVVNLIQAFFFVLVRPFSKNAYRKINRVVAELLWLELIWLIDWWAGVKIQLYTDPETFKLMGKEHALVICNHKSDIDWLVGWILAQRSGCLGSALAVMKKSSKFLPVIGWSMWFSEYLFLERSWAKDENTLKSGFQRLRDFPHAFWLALFVEGTRFTQAKLLAAQEYASSMGLPAPRNVLIPRTKGFVTAVTHMRPFVPAVYDVTLAIPKTSPPPTMLRLFKGQSSVVHIHLKRHLMSDLPKSDDSVAQWCKDAFVVKDNLLDKHKENDSFGDGVLQDTGRPLNSLVVVISWACLLIFGALKFFQWSSILSSWKGLAFSAVGLGIVTVLMQILIQFSQSERSNRPMPSKHAK

SEQ ID NO: 37 - protein, 369 aa, Artificial Sequence
  Description of Artificial Sequence Synthetic polypeptide
MAIPTAAYVVPLGAIFFFSGLLVNLIQAFFFITVWPLSKKTYIRINKVIVELLWLEFVWLADWWAGLKIEVYADAETFQLMGKEHALVICNHKSDIDWLVGWILAQRAGCLGSSFAVTKKSARYLPVVGWSIWFSGAIFLERSWEKDENTLKAGFQRLREFPCAFWLGLFVEGTRFTQAKLLAAQEYASTMGLPFPRNVLIPRTKGFIAAVNHMREFVPAIYDLTFAFPKDSPPPTMLRLLKGQPSVVHVHIKRHLMKDLPEKNEAVAQWCKDVFLVKDKLLDKHKDDGSFGDGELHEIGRPLKSLVVVTTWACLLILGTLKFLLWSSLLSSWKGLIFSATGLAVLTVLMQFLIQSTQSERSNPASLSK

SEQ ID NO: 38 - protein, 315 aa, Artificial Sequence
  Description of Artificial Sequence Synthetic polypeptide
LGLLFFISGLAVNLIQAVCFVFLRPLSKNTYRKINRVLAELLWLQLVWLVDWWAGVKIKVFADRESFNLMGKEHALVICNHKSDIDWLVGWVLAQRSGCLGSSLAVMKKSSKFLPVIGWSMWFSEYLFLERSWAKDESTLKEGLRRLKDFPRPFWLALFVEGTRFTQAKLLAAQEYATSQGLPVPRNVLIPRTKVHVHVKRHLMKELPETDEAVAQWCKDLFVEKDKLLDKHVAEDTFSDQPLQDIGRPVKPLLVVSSWACLVAYGALKFLQWSSLLSSWKGIAVSAVALAIVTILMQIMILFSQSERSIPAKVA

SEQ ID NO: 39 - protein, 357 aa, Artificial Sequence
  Description of Artificial Sequence Synthetic polypeptide
LGLLFFISGLAVNLIQAVCFVFLRPLSKNTYRKINRVLAELLWLQLVWLVDWWAGVKIKVFADRESFNLMGKEHALVICNHKSDIDWLVGWVLAQRSGCLGSSLAVMKKSSKFLPVIGWSMWFSEYLFLERSWAKDESTLKEGLRRLKDFPRPFWLALFVEGTRFTQAKLLAAQEYATSQGLPVPRNVLIPRTKGFVSAVSHMRSFVPAVYDMTVAIPKSSPSPTMLRLFKGQSSVVHVHVKRHLMKELPETDEAVAQWCKDLFVEKDKLLDKHVAEDTFSDQPLQDIGRPVKPLLVVSSWACLVAYGALKFLQWSSLLSSWKGIAVSAVALAIVTILMQIMILFSQSERSIPTKVA

SEQ ID NO: 40 - protein, 345 aa, Artificial Sequence
  Description of Artificial Sequence Synthetic polypeptide
MAIAAAAVVFLFGLLFFTSGLIINLAQAVCFVLIWPLSKNAYRRINRVFAELLLLELLWLFHWRAGAKLKLFADPETFRLFGKEHALVICNHRTDLDWMVGWVLGQHFGCLGSILSVAKKSTKFLPVLGWSMWFSEYLFLERSWAKDKSTLKSHTERLKDYPLPFWLGIFVEGTRFTRAKLLAAQQYAASSGLPVPRNVLIPHTKLHVHIKRYAMKDLPESDDAVAQWCRDIYVEKDAFLDKHNAEDTFSGQEVHHIGRPIKSLLVVISWVVVIIFGALKFLRWSSLLSSWKGKAFSVIGLGIVTLLVNILILSSQAERSNPAKVAPAKLKTELSPSKKVTNKEN

SEQ ID NO: 41 - protein, 387 aa, Artificial Sequence
  Description of Artificial Sequence Synthetic polypeptide
MAIAAAAVVFLFGLLFFTSGLIINLAQAVCFVLIWPLSKNAYRRINRVFAELLLLELLWLFHWRAGAKLKLFADPETFRLFGKEHALVICNHRTDLDWMVGWVLGQHFGCLGSILSVAKKSTKFLPVLGWSMWFSEYLFLERSWAKDKSTLKSHTERLKDYPLPFWLGIFVEGTRFTRAKLLAAQQYAASSGLPVPRNVLIPHTKGFVSSMSHMRSFVPAVYDLTVAFPKTSPPPTLLKLFEGQSVVLHVHIKRYAMKDLPESDDAVAQWCRDIYVEKDAFLDKHNAEDTFSGQEVHHIGRPIKSLLVVISWVVVIIFGALKFLRWSSLLSSWKGKAFSVIGLGIVTLLVNILILSSQAERSNPAKVAPAKLKTELSPSKKVTNKEN

SEQ ID NO: 42 - protein, 382 aa, Artificial Sequence
  Description of Artificial Sequence Synthetic polypeptide
AAAVIVPLGILFFISGLVVNLLQAICYVLIRPLSKNTYRKINRVVAETLWLELVWIVDWWAGVKIQVFADNETFNRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPESDDAIAQWCRDQFVAKDALLDKHIAADTFPGQQEQNIGRPIKSLAVVLSWSCLLILGAMKFLHWSNLFSSWKGIAFSALGLGIITLCMQILIRSSQSERSTPAKVVPAKPKDNHNDSGSSSQTE

SEQ ID NO: 43 - protein, 382 aa, Artificial Sequence
  Description of Artificial Sequence Synthetic polypeptide
AAAVIVPLGILFFISGLVVNLLQAVCYVLVRPMSKNTYRKINRVVAETLWLELVWIVDWWAGVKIQVFADDETFNRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPESDDAIAQWCRDQFVAKDALLDKHIAADTFPGQQEQNIGRPIKSLAVVLSWSCLLILGAMKFLHWSNLFSSWKGIAFSALGLGIITLCMQILIRSSQSERSTPAKVVPAKPKDNHNDSGSSSQTE

SEQ ID NO: 44 - protein, 385 aa, Artificial Sequence
  Description of Artificial Sequence Synthetic polypeptide
MAIAAAVIVPLGLLFFISGLLMNLLQAICYVLVRPLSKNTYRKINRVVAETLWLELVWIVDWWAGVKIKVFADNETFSRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPESDDAIAQWCRDQFVAKDALLDKHIAADTFPGQQEQNIGRPIKSLAVVLSWSCLLILGAMKFLHWSNLFSSWKGIAFSALGLGIITLCMQILIRSSQSERSTPAKVVPAKPKDNHNDSGSSSQTE

SEQ ID NO: 45 - protein, 352 aa, Artificial Sequence
  Description of Artificial Sequence Synthetic polypeptide
INLVVAETLWLELVWIVDWWAGVKIQVFADDETFNRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPESDDAIAQWCRDQFVAKDALLDKHIAADTFPGQKEQNIGRPIKSLAVSLIKTFPWLHPHQLTNIFVLFQVVVSWACLLTLGAMKFLHWSNLFSSWKGIALSAFGLGIITLCMQILIRSSQSERSTPAKVAPAKPK

SEQ ID NO: 46 - protein, 329 aa, Artificial Sequence
  Description of Artificial Sequence Synthetic polypeptide
INLVVAETLWLELVWIVDWWAGVKIQVFADDETFNRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPEPEDEIAQWCRDQFVAKDALLDKHIAADTFPGQKEQNIGRPIKSLAVVVSWACLLTLGAMKFLHWSNLFSSWKGIALSAFGLGIITLCMQILIRSSQSERSTPAKVAPAKPK

SEQ ID NO: 47 - protein, 342 aa, Artificial Sequence
  Description of Artificial Sequence Synthetic polypeptide
INLVVAETLWLELVWIVDWWAGVKIQVFADDETFNRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPESDDAIAQWCRDQFVAKDALLDKHIAADTFPGQQEQNIGRPIKSLAVVLSWSCLLILGAMKFLHWSNLFSSWKGIAFSALGLGIITLCMQILIRSSQSERSTPAKVVPAKPKDNHNDSGSSSQTE

SEQ ID NO: 48 - protein, 267 aa, Artificial Sequence
  Description of Artificial Sequence Synthetic polypeptide
INLVVAETLWLELVWIVDWWAGVKIQVFADDETFNRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPESDDAIAQWCRDQFVAKDALLDKHIAADTFPGQQEQNIGRPIKSLAVSLS

SEQ ID NO: 49 - protein, 288 aa, Artificial Sequence
  Description of Artificial Sequence Synthetic polypeptide
MAIGVAAIVVPLGLLFILSGLMVNLIQAICFILVRPLSKNMYRRVNRVVVELLWLELIWLIDWWGGVKVDVYADSETFQSLGKEHALVVSNHRSDIDWLVGWVLAQRSGCLGSTLAVMKKSSKFLPVIGWSMWFSEYVFLERSWAKDESTLKSGLRRLKDFPRPFWLALFVEGTRFTQAKLLAAREYAASTGLPIPRNVLIPRTKGFVSAVSNMRSFVPAIYDVTVAIPKTQPSPTMLRIFNRQPSVVHVHIKRHSMNQLPQTDEGVGQWCKDIFVAKDALLDRHLAE

SEQ ID NO: 50 - protein, 375 aa, Artificial Sequence
  Description of Artificial Sequence Synthetic polypeptide
MAIGVAAIVVPLGLLFILSGLMVNLIQAICFILVRPLSKNMYRRVNRVVVELLWLELIWLIDWWGGVKVDVYADSETFQSLGKEHALVVSNHRSDIDWLVGWVLAQRSGCLGSTLAVMKKSSKFLPVIGWSMWFSEYVFLERSWAKDESTLKSGLRRLKDFPRPFWLALFVEGTRFTQAKLLAAREYAASTGLPIPRNVLIPRTKGFVSAVSNMRSFVPAIYDVTVAIPKTQPSPTMLRIFNRQPSVVHVHIKRHSMNQLPQTDEGVAQWCKDIFVAKDALLDRHLAEGKFDEKEFKRIRRPIKSLLVISSWSFLLMFGVFKFLKWSALLSTWKGVAVSTTVLLLVTVVMYMFILFSQSERSSPRKVAPSGPENG

SEQ ID NO: 51 - protein, 375 aa, Artificial Sequence
  Description of Artificial Sequence Synthetic polypeptide
MAIGVAAIVVPLGLLFILSGLIINLIQAICFILVRPLSKNMYRKVNRVVVELLWLELIWLIDWWGGVKVDVYADSETFQSLGKEHALVVSNHRSDIDWLVGWVLAQRSGCLGSTLAVMKKSSKFLPVIGWSMWFSEYVFLERSWAKDESTLKSGLQRLKDFPRPFWLALFVEGTRFTQAKLLAAQEYAASTGLPIPRNVLIPRTKGFVSAVSNMRSFVPAIYDVTVAIPKTQPSPTMLRIFNRQPSVVHVHIKRHSMNQLPQTDEGVAQWCKDIFVAKDALLDRHLAEGKFDEKEFKLIRRPIKSLLVISSWSFLLMFGVFKFLKWSALLSTWKGVAVSTAVLLLVTVVMYMFILFSQSERSSPRKVAPIGPENG

SEQ ID NO: 52 - protein, 288 aa, Artificial Sequence
  Description of Artificial Sequence Synthetic polypeptide
MAIGVAAIVVPLGLLFILSGLIINLIQAICFILVRPLSKNMYRKVNRVVVELLWLELIWLIDWWGGVKVDVYADSETFQSLGKEHALVVSNHRSDIDWLVGWVLAQRSGCLGSTLAVMKKSSKFLPVIGWSMWFSEYVFLERSWAKDESTLKSGLQRLKDFPRPFWLALFVEGTRFTQAKLLAAQEYAASTGLPIPRNVLIPRTKGFVSAVSNMRSFVPAIYDVTVAIPKTQPSPTMLRIFNRQPSVVHVHIKRHSMNQLPQTDEGVAQWCKDIFVAKDALLDRHLAE

SEQ ID NO: 53 - protein, 354 aa, Artificial Sequence
  Description of Artificial Sequence Synthetic polypeptide
SLLFFMSGLVVNFIQAVFYVLVRPISKNTYRRINTLVAELLWLELVWVIDWWAGVKVQLYTDTESFRLMGKEHALLICNHRSDIDWLIGWVLAQRCGCLSSSIAVMKKSSKFLPVIGWSMWFSEYLFLERNWAKDENTLKSGLQRLNDFPKPFWLALFVEGTRFTKAKLLAAQEYAASAGLPVPRNVLIPRTKGFVSAVSNMRSFVPAIYDLTVAIPKTTEQPTMLRLFRGKSSVVHVHLKRHLMKDLPKTDDGVAQWCKDQFISKDALLDKHVAEDTFSGLEVQDIGRPMKSLVVVVSWMCLLCLGLVKFLQWSALLSSWKGMMITTFVLGIVTVLMHILIRSSQSEHSTPAK

SEQ ID NO: 54 - protein, 282 aa, Artificial Sequence
  Description of Artificial Sequence Synthetic polypeptide
QRSGCLGSTLAVMKKSSKFLPVIGWSMWFSEYLFLERSWAKDESTLKSGLKRLKDYPLPFWLALFVEGTRFTQAKLLAAQQYAASSGLPVPRNVLIPRTKGFVSSVSHMRSFVPAIYDVTVAIPKMSTPPTMLRIFKGQSSVLHVHLKRHLMKDLPESDDAVAQWCRDIFVEKDALLDKHNAEDTFSGQELQDIGRPVKSLLVVISWAVLVIFGAVKFLQWSSLLSSWKGLAFSGIGLGIVTLLMHILILFSQSERSTPAKVAPAKPKKEGESSKTETEKEN

SEQ ID NO: 55 - protein, 247 aa, Artificial Sequence
  Description of Artificial Sequence Synthetic polypeptide
DWWAGVKIKVFTDHETLSLMGKEHALVISNHKSDIDWLVGWVLAQRSGCLGSTLAVMKKSSKFLPVIGWSMWFSEYLFLERSWAKDESTLKSGLKRLKDYPLPFWLALFVEGTRFTQAKLLAAQQYAASSGLPVPRNVLIPRTKGFVSSVSHMRSFVPAIYDVTVAIPKMSTPPTMLRIFKGQSSVLHVHLKRHLMKDLPESDDAVAQWCRDIFVEKDALLDKHNAEDTFSGQELQDIGRPVKSLLV

SEQ ID NO: 56 - protein, 326 aa, Artificial Sequence
  Description of Artificial Sequence Synthetic polypeptide
DWWAGVKIKVFTDHETLSLMGKEHALVISNHKSDIDWLVGWVLAQRSGCLGSTLAVMKKSSKFLPVIGWSMWFSEYLFLERSWAKDESTLKSGLKRLKDYPLPFWLALFVEGTRFTQAKLLAAQQYAASSGLPVPRNVLIPRTKGFVSSVSHMRSFVPAIYDVTVAIPKMSTPPTMLRIFKGQSSVLHVHLKRHLMKDLPESDDAVAQWCRDIFVEKDALLDKHNAEDTFSGQELQDIGRPVKSLLVVISWAVLVIFGAVKFLQWSSLLSSWKGLAFSGIGLGIVTLLMHILILFSQSERSTPAKVAPAKPKKEGESSKTETEKEN

SEQ ID NO: 57 - protein, 203 aa, Artificial Sequence
  Description of Artificial Sequence Synthetic polypeptide
QRSGCLGSTLAVMKKSSKFLPVIGWSMWFSEYLFLERSWAKDESTLKSGLKRLKDYPLPFWLALFVEGTRFTQAKLLAAQQYAASSGLPVPRNVLIPRTKGFVSSVSHMRSFVPAIYDVTVAIPKMSTPPTMLRIFKGQSSVLHVHLKRHLMKDLPESDDAVAQWCRDIFVEKDALLDKHNAEDTFSGQELQDIGRPVKSLLV

SEQ ID NO: 58 - protein, 376 aa, Artificial Sequence
  Description of Artificial Sequence Synthetic polypeptide
LSLLFFVSGLFVNLVQAVCFVLIRPFSKNTYRRINRVVAELLWLELVWLIDWWAGVKIKVFTDHETLSLMGKEHALVISNHKSDIDWLVGWVLAQRSGCLGSTLAVMKKSSKFLPVIGWSMWFSEYLFLERSWAKDESTLKSGLKRLKDYPLPFWLALFVEGTRFTQAKLLAAQQYAASSGLPVPRNVLIPRTKGFVSSVSHMRSFVPAIYDVTVAIPKMSTPPTMLRIFKGQSSVLHVHLKRHLMKDLPESDDAVAQWCRDIFVEKDALLDKHNAEDTFSGQELQDIGRPVKSLLVVISWAVLVIFGAVKFLQWSSLLSSWKGLAFSGIGLGIVTLLMHILILFSQSERSTPAKVAPAKPKKEGESSKTETEKEN

SEQ ID NO: 59 - protein, 361 aa, Artificial Sequence
  Description of Artificial Sequence Synthetic polypeptide
QAVCFVLIRPFSKNTYRRINRVVAELLWLELVWLIDWWAGVKIKVFTDHETLSLMGKEHALVISNHKSDIDWLVGWVLAQRSGCLGSTLAVMKKSSKFLPVIGWSMWFSEYLFLERSWAKDESTLKSGLKRLKDYPLPFWLALFVEGTRFTQAKLLAAQQYAASSGLPVPRNVLIPRTKGFVSSVSHMRSFVPAIYDVTVAIPKMSTPPTMLRIFKGQSSVLHVHLKRHLMKDLPESDDAVAQWCRDIFVEKDALLDKHNAEDTFSGQELQDIGRPVKSLLVVISWAVLVIFGAVKFLQWSSLLSSWKGLAFSGIGLGIVTLLMHILILFSQSERSTPAKVAPAKPKKEGESSKTETEKEN

SEQ ID NO: 60 - protein, 387 aa, Artificial Sequence
  Description of Artificial Sequence Synthetic polypeptide
MAIAAAAVVFLFGLLFFTSGLIINLAQAVCFVLIWPLSKNAYRRINRVFAELLLLELLWLFHWRAGAKLKLFADPETFRLFGKEHALVICNHRTDLDWMVGWVLGQHFGCLGSILSVAKKSTKFLPVLGWSMWFSEYLFLERSWAKDKSTLKSHTERLKDYPLPFWLGIFVEGTRFTRAKLLAAQQYAASSGLPVPRNVLIPHTKGFVSSMSHMRSFVPAVYDLTVAFPKTSPPPTLLKLFEGQSVVLHVHIKRYAMKDLPESDDAVAQWCRDIYVEKDAFLDKHNAEDTFSGQEVHHIGRPIKSLLVVISWVVVIIFGALKFLRWSSLLSSWKGKAFSVIGLGIVTLLVNILILSSQAERSNPAKVVPAKLKTELSPSKKVTNKEN

SEQ ID NO: 61 - protein, 386 aa, Artificial Sequence
  Description of Artificial Sequence Synthetic polypeptide
MAIPSAAVVFLFGLLFFASGLIINLVQAVCFVLIWPLSKNTCRRINIVFQDMLLSELLWLFHWRAGAKLKFFTDPETYRHMGKEHALVITNHRTDLDWMIGWVLGEHLGCLGSILSVVKKSTKFLPVLGWSMWFSEYLFLERNWAKDKSTFKSHIERLEDFPQPFWFGIFVEGTRFTRAKLLAAQQYAASSGLPVPRNVLIPHTKGFVSSVSHMRSFVPAVYETTMTFPKTSPPPTLLKLFEGQPLVLHIHMKRHAMKDIPESDDAVAQWCRDKFVEKDALLDKHNAEDTFGGLEVHIGRSIKSLMVVICWVVVIIFGALKFLQWSSLLSSWKGIAFIGIGLGIVNLLVHVLILSSQAERSAPTKVAPAKLKTKLLSSKKITNKEN

SEQ ID NO: 62 - protein, 387 aa, Artificial Sequence
  Description of Artificial Sequence Synthetic polypeptide
MAIPSAAVVFLFGLLFFASGLIINLVQAVCFVLIWPLSKNTCRRINIVFQDMLLSELLWLFHWRAGAKLKFFTDPETYRHMGKEHALVITNHRTDLDWMIGWVLGEHLGCLGSILSVVKKSTKFLPVLGWSMWFSEYLFLERNWAKDKSTFKSHIERLEDFPQPFWFGIFVEGTRFTRAKLLAAQQYAASSGLPVPRNVLIPRTKGFVSSVSHMRSFVPAIYDVTVAIPKTSPPPTMLRMFKGQSSVLHVHLKRHLMKDLPESDDAVAQWCRDIFVEKDALLDKHNAEDTFSGQELQDIGRPIKSLVVVISWAALVVFGAVKFLQWSSLLSSWKGLAFSGIGLGIITLLMHILILFSQSERSTPAKVAPAKPKREGESSKTEMDKEN

SEQ ID NO: 63 - protein, 360 aa, Artificial Sequence
  Description of Artificial Sequence Synthetic polypeptide
MAIPAAAVVFLFGLLFFPSGLIINLFQAVCFVLIWPFSRNTCRRINIVFQEMLLSELLWLFHWRAGAKLKLFADPETYRHMGKEHALLITNHRTDLDWMIGWALGQHLGCLGSILSVVKKSTKFLPSHIERLEDFPQPFWMAIFVEGTRFTRAKLLAAQQYAASSGLPVPRNVLIPRTKGFVSCVSHMRSFVPAVYETTMTFPKTSPPPTLLKLFEGQPIVLHVHMKRHAMKDIPESDEAVAQWCRDKFVEKDSLLDKHNAGDTFSCQEIHIGRPIKSLMVVISWVVVIIFGALKFLQWSSLLSSWKGIAFSGIGLGIVTLLVHILILSSQAERSTPAKVAPAKLKTELSSSTKVTNKEN

SEQ ID NO: 64 - protein, 386 aa, Artificial Sequence
  Description of Artificial Sequence Synthetic polypeptide
MAIPAAAVVFLFGLLFFPSGLIINLFQAVCFVLIWPFSRNTCRRINIVFQEMLLSELLWLFHWRAGAKLKLFADPETYRHMGKEHALLITNHRTDLDWMIGWALGQHLGCLGSILSVVKKSTKFLPVLGWSMWFSEYLFLERNWAKDKSTFKSHIERLEDFPQPFWMAIFVEGTRFTRAKLLAAQQYAASSGLPVPRNVLIPRTKGFVSCVSHMRSFVPAVYETTMTFPKTSPPPTLLKLFEGQPIVLHVHMKRHAMKDIPESDEAVAQWCRDKFVEKDSLLDKHNAGDTFSCQEIHIGRPIKSLMVVISWVVVIIFGALKFLQWSSLLSSWKGIAFSGIGLGIVTLLVHILILSSQAERSTPAKVAPAKLKTELSSSTKVTNKEN

SEQ ID NO: 65 - protein, 376 aa, Artificial Sequence
  Description of Artificial Sequence Synthetic polypeptide
LSLLFFVSGLIVNLVQAVCFVLIRPLSKNTYRRINRVVAELLWLELVWLIDWWAGVKIKVFTDHETFRLMGTEHALVISNHKSDIDWLVGWVLAQRSGCLGSTLAVMKKSSKFLPVIGWSMWFSEYLFLERSWAKDESTLKSGLNRLKDYPLPFWLALFVEGTRFTRAKLLAAQQYAASSGLPVPRNVLIPRTKGFVSSVSHMRSFVPAIYDVTVAIPKTSPPPTMLRMFKGQSSVLHVHLKRHLMKDLPESDDAVAQWCRDIFVEKDALLDKHNAEDTFSGQELQDIGRPIKSLVVVISWAALVVFGAVKFLQWSSLLSSWKGLAFSGIALGIITLLMHILILFSQSERSTPAKVAPAKPKKEGESSKTETDKEN

SEQ ID NO: 66 - protein, 386 aa, Artificial Sequence
  Description of Artificial Sequence Synthetic polypeptide
MAIPAAAVIFLFSILFFASGLIINLVQAVCFVLIWPLSKNTCRRINLVFQEMLLSELLGLFHWRAGAKLKLYTDPETYPLLGKEHALLMINHRTDLDWMIGWVLGQHLGCLGSILSVVKKSTKFLPVLGWSMWFSEYLFLERNWAKDKSTFKSHIERLEDFPQPFWMAIFVEGTRFTRAKLLAAQQYAASSGLPVPRNVLIPHTKGFVSTVSHMRSFVPAVYDTTLTFPKTSPPPTLLNLFAGQPIVLHIHIKRHAMKDIPESDDAVAQWCRDKFVEKDALLDKHNAEDAFSDQEFPISRSIKSLMVVISWVMVIIFGALKFLQWSSLLSSWKGKAFSVIAVGIVTLLMHMSILSSQAERSNPAKVALPKLKTELPSSKKVLNKEN

SEQ ID NO: 67 - protein, 386 aa, Artificial Sequence
  Description of Artificial Sequence Synthetic polypeptide
MAIPAAAVIFLFSILFFASGLIINLVQAVCFVLIWPLSKNTCRRINLVFQEMLLSELLGLFHWRAGAKLKLYTDPETYPLLGKEHALLMINHRTDLDWMIGWVLGQHLGCLGSILSVVKKSTKFLPVLGWSMWFSEYLFLERNWAKDKSTFKSHIERLEDFPQPFWMAIFVEGTRFTRAKLLAAQQYAASSGLPVPRNVLIPHTKGFVSTVSHMRSFVPAVYDTTLTFPKTSPPPTLLNLFAGQPIVLHIHIKRHAMKDIPESDDAVAQWCRDKFVEKDALLDKHNAEDAFSDQEFPISRSIKSLMVVISWVMVIIFGALKFLQWSSLLSSWKGIAFSGIGLGIVTLLMHILILSSQAERSTPAKVAQAKVKTELPSSTKVTNKGN

SEQ ID NO: 68 - protein, 386 aa, Artificial Sequence
  Description of Artificial Sequence Synthetic polypeptide
MAIPAAAVIFLFGILFFASGLIINLVQAVCFVLIWPLSKNTCRRINLVFQEMLLSELLWLFHWRAGAELKLFTDPETYRLLGKEHALVMTNHRTDLDWMIGWVTGQHLGCLGSILSIAKKSTKFLPVLGWSMWFSEYLFLERNWAKDKSTFKSHIERLEDFPQPFWMAIFVEGTRFTRAKLLAAQQYAASSGLPVPRNVLIPHTKGFVSSVCHMRSFVPAVYDTTLTFPKNSPPPTLLNLFAGQPIVLHIHIKRHAMKDMPKSDDAVAQWCRDKFVKKDALLDKHNTEDTFSDQEFPIGRPIKSLMVVISWVVVIIFGTLKFLQWSSLLSSWKGIAFSGIGLGIVTLLVHILILSSQAERSTPPKVAPAKLKTELSSTTKVINKGN

SEQ ID NO: 69 - protein, 345 aa, Artificial Sequence
  Description of Artificial Sequence Synthetic polypeptide
LGLLFFVSGLIVNLVQAVCFVLIRPLSKNTYRRLNRVVAELLWLELVWLIDWWAGVKIKVFTDHETFHLMGKEHALVICNHKSDIDWLVGWVLAQRSGCLGSTLAVMKKSSKFLPVIGWSMWFSEYLFLERSWAKDESTLKSGLNRLKDYPLPFWLALFVEGTRFTRAKLLAAQQYAASSGLPVPRNVLIPRTKGFVSSVSHMRSFVPAIYDVTVAIPKTSPPPTMLRMFKGQSSVDALLDKHNADDTFSGQELHDIGRPIKSLLVVISWAVLVVFGAVKFLQWSSLLSSWKGIAFSGIGLGIVTLLVHILILSSQAERSTSAKVAQAKVKTELSSSKKVKNKGN

SEQ ID NO: 70 - protein, 376 aa, Artificial Sequence
  Description of Artificial Sequence Synthetic polypeptide
LGLLFFVSGLIVNLVQAVCFVLIRPLSKNTYRRLNRVVAELLWLELVWLIDWWAGVKIKVFTDHETFHLMGKEHALVICNHKSDIDWLVGWVLAQRSGCLGSTLAVMKKSSKFLPVIGWSMWFSEYLFLERSWAKDESTLKSGLNRLKDYPLPFWLALFVEGTRFTRAKLLAAQQYAASSGLPVPRNVLIPRTKGFVSSVSHMRSFVPAIYDVTVAIPKTSPPPTMLRMFKGQSSVLHVHLKRHLMKDLPESDDAVAQWCRDIFVEKDVLLDKHNAEDTFSGQELQDIGRPVKSLLVVISWTLLVIFGAVKFLQWSSLLSSWKGLAFSGIGLGIVTLLMHILILFSQSERSTPAKVAPAKPKKEGESSKMETDKEN

SEQ ID NO: 71 - protein, 288 aa, Artificial Sequence
  Description of Artificial Sequence Synthetic polypeptide
LAGWMGSSSGCLGSTLAVMKKSSKFLPVIGWSMWFSEYLFLERSWAKDESTLKSGLNRLKDYPLPFWLALFVEGTRFTRAKLLAAQQYAASLGLPVPRNVLIPRTKGFVSSVSHMRSFVPAIYDVTVAIPKTSPPPTMIRMFKGQSSVLHVHLKRHVMKDLPESDDAVAQWCRDIFVEKDALLDKHNAEDTFSGQELQDTGRPIKSLLVVISWAVLEVFGAVKFLQWSSLLSSWKGLAFSGIGLGIITLLMHILILFSQSERSTPAKVAPAKPKNEGESSKAEMEKEK

SEQ ID NO: 72 - protein, 313 aa, Artificial Sequence
  Description of Artificial Sequence Synthetic polypeptide
LAGWMGSSSGCLGSTLAVMKKSSKFLPVIGWSMWFSEYLFLERSWAKDESTLKSGLNRLKDYPLPFWLALFVEGTRFTRAKLLAAQQYAASLGLPVPRNVLIPRTKGFVSSVSHMRSFVPAIYDVTVAIPKTSPPPTMIRMFKGQSSVLHVHLKRHVMKDLPESDDAVAQWCRDIFVEKDALLDKHNAEDTFSGQELQDTGRPIKSLLVRCFLVLSLIYLNGIMLKLRGPCLQVVISWAVLEVFGAVKFLQWSSLLSSWKGLAFSGIGLGIITLLMHILILFSQSERSTPAKVAPAKPKNEGESSKAEMEKEK

SEQ ID NO: 73 - protein, 288 aa, Artificial Sequence
  Description of Artificial Sequence Synthetic polypeptide
LAGWMGSSSGCLGSTLAVMKKSSKFLPVIGWSMWFSEYLFLERSWAKDESTLKSGLNRLKDYPLPFWLALFVEGTRFTRAKLLAAQQYAASLGLPVPRNVLIPRTKGFVSSVSHMRSFVPAIYDVTVAIPKTSPPPTMIRMFKGQSSVLHVHLKRHVMKDLPESDDAVAQWCRDIFVEKDALLDKHNAEDTFSGQELQDTGRPIKSLLVVTSWAVLVISGAVKFLQWSSLLSSWKGLAFSGIGLGIVTLLMHILILFSQSERSTPAKVAPAKPKKEGESSKTEKDKEN

SEQ ID NO: 74 - protein, 376 aa, Artificial Sequence
  Description of Artificial Sequence Synthetic polypeptide
LGLLFFVSGLIVNLVQAVCFVLIRPLSKNTYRRINRVVAELLWLELVWLIDWWAGVKIKVFTDHETLSLMGKEHALVICNHKSDIDWLVGWVLAQRSGCLGSTLAVMKKSSKFLPVIGWSMWFSEYLFLERSWAKDENTLKSGLNRLKDYPLPFWLALFVEGTRFTRAKLLAAQQYATSSGLPVPRNVLIPRTKGFVSSVSHMRSFVPAIYDVTVAIPKTSPPPTMLRMFKGQSSVLHVHLKRHLMKDLPESDDAVAQWCRDIFVEKDALLDKHNAEDTFSGQELQDTGRPIKSLLVVISWAVLVIFGAVKFLQWSSLLSSWKGLAFSGVGLGIITLLMHILILFSQSERSTPAKVAPAKPKKDGESSKTEIEKEN

SEQ ID NO: 75 - protein, 387 aa, Artificial Sequence
  Description of Artificial Sequence Synthetic polypeptide
MAIAAAAVIVPVSLLFFVSGLIVNLVQAVCFVLIRPLFKNTYRRINRVVAELLWLELVWLIDWWAGVKIKVFTDHETFHLMGKEHALVICNHKSDIDWLVGWVLAQRSGCLGSTLAVMKKSSKFLPVIGWSMWFSEYLFLERNWAKDESTLKSGLNRLKDYPLPFWLALFVEGTRFTRAKLLAAQQYAASSGLPVPRNVLIPRTKGFVSSVSHMRSFVPAIYDVTVAIPKTSPPPTLLRMFKGQSSVLHVHLKRHQMNDLPESDDAVAQWCRDIFVEKDALLDKHNAEDTFSGQELQDTGRPIKSLLIVISWAVLVVFGAVKFLQWSSLLSSWKGLAFSGIGLGVITLLMHILILFSQSERSTPAKVAPAKPKIEGESSKTEMEKEH

SEQ ID NO: 76 - protein, 387 aa, Artificial Sequence
  Description of Artificial Sequence Synthetic polypeptide
MTIASAAVVFLFGILLFTSGLIINLFQAFCSVLVWPLSKNAYRRINRVFAEFLPLEFLWLFHWWAGAKLKLFTDPETFRLMGKEHALVIINHKIELDWMVGWVLGQHLGCLGSILSVAKKSTKFLPVFGWSLWFSEYLFLERNWAKDKKTLKSHIERLKDYPLPFWLIIFVEGTRFTRTKLLAAQQYAASAGLPVPRNVLIPHTKGFVSSVSHMRSFVPAIYDVTVAFPKTSPPPTMLKLFEGHFVELHVHIKRHAMKDLPESEDAVAQWCRDKFVEKDALLDKHNAEDTFSGQEVHHVGRPIKSLLVVISWVVVIIFGALKFLQWSSLLSSWKGIAFSVIGLGTVALLMQILILSSQAERSIPAKETPANLKTELSSSKKVTNKEN

SEQ ID NO: 77 - protein, 375 aa, Artificial Sequence
  Description of Artificial Sequence Synthetic polypeptide
MAIGAAAIVVPLGLLFMLSGLMVNLIQAICFILVRPLSKNMYRRVNRVVVELLWLELIWLIDWWGGVKVDVYADSETFQSLGKEHALVVSNHKSDIDWLVGWVLAQRSGCLGSTLAVMKKSSKFLPVIGWSMWFSEYVFLERSWAKDESTLKSGLQRLKDFPRPFWLALFVEGTRFTQAKLLAAQEYAASTGLPIPRNVLIPRTKGFVSAVSNMRSFVPAIYDVTVAIPKTQPSPTMLRIFNRQPSVVHVRIKRHSMNQLPPTDEGVAQWCKDIFVAKDALLDRHLAEGKFDEKEFKRIRRPIKSLLVISSWSFLLLFGVFKFLKWSALLSTWKGVAVSTAVLLLVTVVMYMFILFSQSERSSPRKVAPSGPENG

SEQ ID NO: 78 - protein, 384 aa, Artificial Sequence
  Description of Artificial Sequence Synthetic polypeptide
MAIPAAVVIFLFGLLFFSSGLIINLFQALCFVLIWPLSKNAYRRINRVFAELLLSELLCLFDWWAGAKLKLFTDPETFRLMGKEHALVIINHMTELDWMVGWVMGQHFGCLGSILSVAKKSTKFLPVLGWSMWFTEYLYIERSWDKDKSTLKSHIERLKDYPLPFWLVIFAEGTRFTRTKLLAAQQYAASSGLPVPRNVLIPRTKGFVSCVNHMRSFVPAVYDLTVAFPKTSPPPTLLNLFEGQSVVLHVHIKRHAMKDLPESDDAVAQWCRDKFVEKDALLDKHNAEDTFSSQEVHHTGSRPIKSLLVVISWVVVITFGALKFLQWSSWKGKAFSVIGLGIVTLLMHMLILSSQAERSKPAKVTQAKLKTELSISKKVTDKEN

SEQ ID NO: 79 - protein, 384 aa, Artificial Sequence
  Description of Artificial Sequence Synthetic polypeptide
MAIAAAAVIFLFGLLFFASGLIINLFQALCFVLIRPLSKNAYRRINRVFAELLLSELLCLFDWWAGAKLKLFTDPETLRLMGKEHALIIINHMTELDWMVGWVMGQHFGCLGSIISVAKKSTKFLPVLGWSMWFSEYLYLERSWAKDKSTLKSHIERLKDYPLPFWLVIFVEGTRFTRTKLLAAQEYAASSGLPVPRNVLIPRTKGFVSCVNHMRSFVPAVYDVTVAFPKTSPQPTLLNLFEGRSIVLHVHIKRHAMKDLPESDDAVAQWCRDKFVEKDALLDKHNAEDTFSGQEVHHTGRRPIKSLLVVMSWVVVTTFGALKFLQWSSWKGKAFSVIGLGIVTLLMHVLILSSQAERSNPAKVVQAELNTELSISKKVTNKGN

SEQ ID NO: 80 - protein, 373 aa, Artificial Sequence
  Description of Artificial Sequence Synthetic polypeptide
MAIPAAAVIFLFGLIFFASGLIINLFQALCFVLIWPLWKNAYRRINRVFAELLLSELLCLFDWWAGAKLKLFTDPETFRLMGKEHALVIINHMTELDWMVGWVMGQHFGCLGSILSVAKKSTKFLPVLGWSMWFTEYLYIERSWDKDKSTLKSHIERLKDYPLPFWLVIFAEGTRFTRTKLLAAQQYAASSGLPVPRNVLIPRTKGFVSSVSHMRSFVPAIYDVTVAIPKTSPPPTLIRMFKGQSSVLHVHLKRHVMKDLPESDDAVAQWCRDIFVEKDALLDKHNAEDTFSGQELQDTGRPIKSLLVVISWAVLEVFGAVKFLQWSSLLSSWKGLAFSGIGLGIITLLMHILILFSQSERSTPAKVAPAKAK

SEQ ID NO: 81 - protein, 398 aa, Artificial Sequence
  Description of Artificial Sequence Synthetic polypeptide
MAIPAAAVIFLFGLIFFASGLIINLFQALCFVLIWPLWKNAYRRINRVFAELLLSELLCLFDWWAGAKLKLFTDPETFRLMGKEHALVIINHMTELDWMVGWVMGQHFGCLGSILSVAKKSTKFLPVLGWSMWFTEYLYIERSWDKDKSTLKSHIERLKDYPLPFWLVIFAEGTRFTRTKLLAAQQYAASSGLPVPRNVLIPRTKGFVSSVSHMRSFVPAIYDVTVAIPKTSPPPTLIRMFKGQSSVLHVHLKRHVMKDLPESDDAVAQWCRDIFVEKDALLDKHNAEDTFSGQELQDTGRPIKSLLVRCFLVLSLIYLNGIILKLCGLCLQVVISWAVLEVFGAVKFLQWSSLLSSWKGLAFSGIGLGIITLLMHILILFSQSERSTPAKVAPAKAK

SEQ ID NO: 82 - protein, 387 aa, Artificial Sequence
  Description of Artificial Sequence Synthetic polypeptide
MAIPAAAVIFLFGLIFFASGLIINLFQALCFVLIWPLWKNAYRRINRVFAELLLSELLCLFDWWAGAKLKLFTDPETFRLMGKEHALVIINHMTELDWMVGWVMGQHFGCLGSILSVAKKSTKFLPVLGWSMWFTEYLYIERSWDKDKSTLKSHIERLKDYPLPFWLVIFAEGTRFTRTKLLAAQQYAASSGLPVPRNVLIPRTKGFVSSVSHMRSFVPAIYDVTVAIPKTSPPPTLIRMFKGQSSVLHVHLKRHVMKDLPESDDAVAQWCRDIFVEKDALLDKHNAEDTFSGQELQDTGRPIKSLLVVISWAVLEVFGAVKFLQWSSLLSSWKGLAFSGIGLGIITLLMHILILFSQSERSTPAKVAPAKAKMEGESSKTEMEMEK

SEQ ID NO: 83 - protein, 412 aa, Artificial Sequence
  Description of Artificial Sequence Synthetic polypeptide
MAIPAAAVIFLFGLIFFASGLIINLFQALCFVLIWPLWKNAYRRINRVFAELLLSELLCLFDWWAGAKLKLFTDPETFRLMGKEHALVIINHMTELDWMVGWVMGQHFGCLGSILSVAKKSTKFLPVLGWSMWFTEYLYIERSWDKDKSTLKSHIERLKDYPLPFWLVIFAEGTRFTRTKLLAAQQYAASSGLPVPRNVLIPRTKGFVSSVSHMRSFVPAIYDVTVAIPKTSPPPTLIRMFKGQSSVLHVHLKRHVMKDLPESDDAVAQWCRDIFVEKDALLDKHNAEDTFSGQELQDTGRPIKSLLVRCFLVLSLIYLNGIILKLCGLCLQVVISWAVLEVFGAVKFLQWSSLLSSWKGLAFSGIGLGIITLLMHILILFSQSERSTPAKVAPAKAKMEGESSKTEMEMEK

SEQ ID NO: 84 - protein, 387 aa, Artificial Sequence
  Description of Artificial Sequence Synthetic polypeptide
MAIAAAPVIFLFGLLFFASGLIINLFQAICFVLIWPLSKNAYRRINRVFAELLLSELLCLFDWWAGAKLKLFTDPETFRLMGKEHALVITNHKIDLDWMIGWILGQHFGCLGSVISIAKKSTKFLPIFGWSLWFSEYLFLERNWAKDKRTLKSHIERMKDYPLPLWLILFVEGTRFTRTKLLAAQQYAASSGLPVPRNVLIPHTKGFVSSVSHMRSFVPAIYDVTVAIPKTSPPPTLIRMFKGQSSVLHVHLKRHLMKDLPESDDAVAQWCRDIFVEKDALLDKHNAEDTFSGQELQETGRPIKSLLVVISWAVLEVYGAVKFLQWSSLLSSWKGLAFSGIGLGLITLLMHILILFSQSERSTPAKVAPAKPKKEGESSKTEMEKEK

SEQ ID NO: 85 - protein, 382 aa, Artificial Sequence
  Description of Artificial Sequence Synthetic polypeptide
MHVLLEMVTFRFSSFFVFDNVQALCFVLIWPLSKSAYRKINRVFAELLLSELLCLFDWWAGAKLKLFTDPETFRLMGKEHALVITNHKIDLDWMIGWILGQHFGCLGSVISIAKKSTKFLPIFGWSLWFSEYLFLERNWAKDKRTLKSHIERMKDYPLPLWLILFVEGTRFTRTKLLAAQQYAASSGLPVPRNVLIPHTKGFVSSVSHMRSFVPAVYDVTVAFPKTSPPPTMLSLFEGQSVVLHVHIKRHAMKDLPDSDDAVAQWCRDKFVEKDALLDKHNAEDTFSGQEVHHVGRPIKSLLVVISWMVVIIFGALKFLQWSSLLSSWKGKAFSAIGLGIATLLMHVLVVFSQADRSNPAKVPPAKLNTELSSSKKVTNKEN

SEQ ID NO: 86 - DNA, 7194 bp, Artificial Sequence
  Description of Artificial Sequence Synthetic polynucleotide
gtttaaacgccggtcaccacccgcatgctcgtactacagcgcacgcaccgcttcgtgatccaccgggtgaacgtagtcctcgacggaaacatctggttcgggcctcctgcttgcactcccgcccatgccgacaacctttctgctgttaccacgacccacaatgcaacgcgacacgaccgtgtgggactgatcggttcactgcacctgcatgcaattgtcacaagcgcttactccaattgtattcgtttgttttctgggagcagttgctcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaactagtatgcagaccgcccaccagcgcccccccaccgagggccactgcttcggcgcccgcctgcccaccgcctcccgccgcgccgtgcgccgcgcctggtcccgcatcgcccgcgggcgcgccgccgccgccgccgacgccaaccccgcccgccccgagcgccgcgtggtgatcaccggccagggcgtggtgacctccctgggccagaccatcgagcagttctactcctccctgctggagggcgtgtccggcatctcccagatccagaagttcgacaccaccggctacaccaccaccatcgccggcgagatcaagtccctgcagctggacccctacgtgcccaagcgctgggccaagcgcgtggacgacgtgatcaagtacgtgtacatcgccggcaagcaggccctggagtccgccggcctgcccatcgaggccgccggcctggccggcgccggcctggaccccgccctgtgcggcgtgctgatcggcaccgccatggccggcatgacctccttcgccgccggcgtggaggccctgacccgcggcggcgtgcgcaagatgaaccccttctgcatccccttctccatctccaacatgggcggcgccatgctggccatggacatcggcttcatgggccccaactactccatctccaccgcctgcgccaccggcaactactgcatcctgggcgccgccgaccacatccgccgcggcgacgccaacgtgatgctggccggcggcgccgacgccgccatcatcccctccggcatcggcggcttcatcgcctgcaaggccctgtccaagcgcaacgacgagcccgagcgcgcctcccgcccctgggacgccgaccgcgacggcttcgtgatgggcgagggcgccggcgtgctggtgctggaggagctggagcacgccaagcgccgcggcgccaccatcctggccgagctggtgggcggcgccgccacctccgacgcccaccacatgaccgagcccgacccccagggccgcggcgtgcgcctgtgcctggagcgcgccctggagcgcgcccgcctggcccccgagcgcgtgggctacgtgaacgcccacggcacctccacccccgccggcgacgtggccgagtaccgcgccatccgcgccgtgatcccccaggactccctgcgcatcaactccaccaagtccatgatcggccacctgctgggcggcgccggcgccgtggaggccgtggccgccatccaggccctgcgcaccggctggctgcaccccaacctgaacctggagaaccccgcccccggcgtggaccccgtggtgctggtgggcccccgcaaggagcgcgccgaggacctggacgtggtgctgtccaactccttcggcttcggcggccacaactcctgcgtgatcttccgcaagtacgacgagatggactacaaggaccacgacggcgactacaaggaccacgacatcgactacaaggacgacgacgacaagtgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctaggtgatatccatcttaaggatctaagtaagattcgaagcgctcgaccgtgccggacggactgcagccccatgtcgtagtgaccgccaatgtaagtgggctggcgtttccctgtacgtgagtcaacgtcactgcacgcgcaccaccctctcgaccggcaggaccaggcatcgcgagatacagcgcgagccagacacggagtgccgagctatgcgcacgctccaactaggtaccagtttaggtccagcgtccgtggggggggacgggctgggagcttgggccgggaagggcaagacgatgcagtccctctggggagtcacagccgactgtgtgtgttgcactgtgcggcccgcagcactcacacgcaaaatgcctggccgacaggcaggccctgtccagtgcaacatccacggtccctctcatcaggctcaccttgctcattgacataacggaatgcgtaccgctctttcagatctgtccatccagagaggggagcaggctccccaccgacgctgtcaaacttgcttcctgcccaaccgaaaacattattgtttgagggggggggggggggggcagattgcatggcgggatatctcgtgaggaacatcactgggacactgtggaacacagtgagtgcagtatgcagagcatgtatgctaggggtcagcgcaggaagggggcctttcccagtctcccatgccactgcaccgtatccacgactcaccaggaccagcttcttgatcggcttccgctcccgtggacaccagtgtgtagcctctggactccaggtatgcgtgcaccgcaaaggccagccgatcgtgccgattcctgggtggaggatatgagtcagccaacttggggctcagagtgcacactggggcacgatacgaaacaacatctacaccgtgtcctccatgctgacacaccacagcttcgctccacctgaatgtgggcgcatgggcccgaatcacagccaatgtcgctgctgccataatgtgatccagaccctctccgcccagatgccgagcggatcgtgggcgctgaatagattcctgtttcgatcactgtttgggtcctttccttttcgtctcggatgcgcgtctcgaaacaggctgcgtcgggctttcggatcccttttgctccctccgtcaccatcctgcgcgcgggcaagttgcttgaccctgggctgataccagggttggagggtattaccgcgtcaggccattcccagcccggattcaattcaaagtctgggccaccaccctccgccgctctgtctgatcactccacattcgtgcatacactacgttcaagtcctgatccaggcgtgtctcgggacaaggtgtgcttgagtttgaatctcaaggacccactccagcacagctgctggttgaccccgccctcgcaatctagaatggccgcgtccgtccactgcaccctgatgtccgtggtctgcaacaacaagaaccactccgcccgccccaagctgcccaactcctccctgctgcccggcttcgacgtggtggtccaggccgcggccacccgcttcaagaaggagacgacgaccacccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcctgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgacaattgacggagcgtcgtgcgggagggagtgtgccgagcggggagtcccggtctgtgcgaggcccggcagctgacgctggcgagccgtacgccccgagggtccccctcccctgcaccctcttccccttccctctgacggccgcgcctgttcttgcatgttcagcgacggatcctagggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtcctgggcgaagaacgagggaatttgtgggtaaaacaagcatcgtctctcaggccccggcgcagtggccgttaaagtccaagaccgtgaccaggcagcgcagcgcgtccgtgtgcgggccctgcctggcggctcggcgtgccaggctcgagagcagctccctcaggtcgccttggacggcctctgcgaggccggtgagggcctgcaggagcgcctcgagcgtggcagtggcggtcgtatccgggtcgccggtcaccgcctgcgactcgccatccgaagagcgtttaaac

SEQ ID NO: 87 - DNA, 7081 bp, Artificial Sequence
  Description of Artificial Sequence Synthetic polynucleotide
gctcttcgccgccgccactcctgctcgagcgcgcccgcgcgtgcgccgccagcgccttggccttttcgccgcgctcgtgcgcgtcgctgatgtccatcaccaggtccatgaggtctgccttgcgccggctgagccactgcttcgtccgggcggccaagaggagcatgagggaggactcctggtccagggtcctgacgtggtcgcggctctgggagcgggccagcatcatctggctctgccgcaccgaggccgcctccaactggtcctccagcagccgcagtcgccgccgaccctggcagaggaagacaggtgaggggggtatgaattgtacagaacaaccacgagccttgtctaggcagaatccctaccagtcatggctttacctggatgacggcctgcgaacagctgtccagcgaccctcgctgccgccgcttctcccgcacgcttctttccagcaccgtgatggcgcgagccagcgccgcacgctggcgctgcgcttcgccgatctgaggacagtcggggaactctgatcagtctaaacccccttgcgcgttagtgttgccatcctttgcagaccggtgagagccgacttgttgtgcgccaccccccacaccacctcctcccagaccaattctgtcacctttttggcgaaggcatcggcctcggcctgcagagaggacagcagtgcccagccgctgggggttggcggatgcacgctcaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcctgaagaatgggaggcaggtgttgttgattatgagtgtgtaaaagaaaggggtagagagccgtcctcagatccgactactatgcaggtagccgctcgcccatgcccgcctggctgaatattgatgcatgcccatcaaggcaggcaggcatttctgtgcacgcaccaagcccacaatcttccacaacacacagcatgtaccaacgcacgcgtaaaagttggggtgctgccagtgcgtcatgccaggcatgatgtgctcctgcacatccgccatgatctcctccatcgtctcgggtgtttccggcgcctggtccgggagccgttccgccagatacccagacgccacctccgacctcacggggtacttttcgagcgtctgccggtagtcgacgatcgcgtccaccatggagtagccgaggcgccggaactggcgtgacggagggaggagagggaggagagagaggggggggggggggggggatgattacacgccagtctcacaacgcatgcaagacccgtttgattatgagtacaatcatgcactactagatggatgagcgccaggcataaggcacaccgacgttgatggcatgagcaactcccgcatcatatttcctattgtcctcacgccaagccggtcaccatccgcatgctcatattacagcgcacgcaccgcttcgtgatccaccgggtgaacgtagtcctcgacggaaacatctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctcttgttttccagaaggagttgctccttgagcctttcattctcagcctcgataacctccaaagccgctctaattgtggagggggttcgaatttaaaagcttggaatgttggttcgtgcgtctggaacaagcccagacttgttgctcactgggaaaaggaccatcagctccaaaaaacttgccgctcaaaccgcgtacctctgctttcgcgcaatctgccctgttgaaatcgccaccacattcatattgtgacgcttgagcagtctgtaattgcctcagaatgtggaatcatctgccccctgtgcgagcccatgccaggcatgtcgcgggcgaggacacccgccactcgtacagcagaccattatgctacctcacaatagttcataacagtgaccatatttctcgaagctccccaacgagcacctccatgctctgagtggccaccccccggccctggtgcttgcggagggcaggtcaaccggcatggggctaccgaaatccccgaccggatcccaccacccccgcgatgggaagaatctctccccgggatgtgggcccaccaccagcacaacctgctggcccaggcgagcgtcaaaccataccacacaaatatccttggcatcggccctgaattccttctgccgctctgctacccggtgcttctgtccgaagcaggggttgctagggatcgctccgagtccgcaaacccttgtcgcgtggcggggcttgttcgagcttgaagagc

SEQ ID NO: 88 - DNA, 6029 bp, Artificial Sequence
  Description of Artificial Sequence Synthetic polynucleotide
gctcttccgctaacggaggtctgtcaccaaatggaccccgtctattgcgggaaaccacggcgatggcacgtttcaaaacttgatgaaatacaatattcagtatgtcgcgggcggcgacggcggggagctgatgtcgcgctgggtattgcttaatcgccagcttcgcccccgtcttggcgcgaggcgtgaacaagccgaccgatgtgcacgagcaaatcctgacactagaagggctgactcgcccggcacggctgaattacacaggcttgcaaaaataccagaatttgcacgcaccgtattcgcggtattttgttggacagtgaatagcgatgcggcaatggcttgtggcgttagaaggtgcgacgaaggtggtgccaccactgtgccagccagtcctggcggctcccagggccccgatcaagagccaggacatccaaactacccacagcatcaacgccccggcctatactcgaaccccacttgcactctgcaatggtatgggaaccacggggcagtcttgtgtgggtcgcgcctatcgcggtcggcgaagaccgggaaggtaccgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatgactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacgtactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagaattcctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccatggccgccgccgtgatcgtgcccctgggcatcctgttcttcatctccggcctggtggtgaacctgctgcaggccatctgctacgtgctgatccgccccctgtccaagaacacctaccgcaagatcaaccgcgtggtggccgagaccctgtggctggagctggtgtggatcgtggactggtgggccggcgtgaagatccaggtgttcgccgacaacgagaccttcaaccgcatgggcaaggagcacgccctggtggtgtgcaaccaccgctccgacatcgactggctggtgggctggatcctggcccagcgctccggctgcctgggctccgccctggccgtgatgaagaagtcctccaagttcctgcccgtgatcggctggtccatgtggttctccgagtacctgttcctggagcgcaactgggccaaggacgagtccaccctgaagtccggcctgcagcgcctgaacgacttcccccgccccttctggctggccctgttcgtggagggcacccgcttcaccgaggccaagctgaaggccgcccaggagtacgccgcctcctccgagctgcccgtgccccgcaacgtgctgatcccccgcaccaagggcttcgtgtccgccgtgtccaacatgcgctccttcgtgcccgccatctacgacatgaccgtggccatccccaagacctcccccccccccaccatgctgcgcctgttcaagggccagccctccgtggtgcacgtgcacatcaagtgccactccatgaaggacctgcccgagtccgacgacgccatcgcccagtggtgccgcgaccagttcgtggccaaggacgccctgctggacaagcacatcgccgccgacaccttccccggccagcaggagcagaacatcggccgccccatcaagtccctggccgtggtgctgtcctggtcctgcctgctgatcctgggcgccatgaagttcctgcactggtccaacctgttctcctcctggaagggcatcgccttctccgccctgggcctgggcatcatcaccctgtgcatgcagatcctgatccgctcctcccagtccgagcgctccacccccgccaaggtggtgcccgccaagcccaaggacaaccacaacgactccggctcctcctcccagaccgaggtggagaagcagaagtgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctcagcggcgacggtcctgctaccgtacgacgttgggcacgcccatgaaagtttgtataccgagcttgttgagcgaactgcaagcgcggctcaaggatacttgaactcctggattgatatcggtccaataatggatggaaaatccgaacctcgtgcaagaactgagcaaacctcgttacatggatgcacagtcgccagtccaatgaacattgaagtgagcgaactgttcgcttcggtggcagtactactcaaagaatgagctgctgttaaaaatgcactctcgttctctcaagtgagtggcagatgagtgctcacgccttgcacttcgctgcccgtgtcatgccctgcgccccaaaatttgaaaaaagggatgagattattgggcaatggacgacgtcgtcgctccgggagtcaggaccggcggaaaataagaggcaacacactccgcttcttagctcttc

SEQ ID NO: 89 - DNA, 1176 bp, Artificial Sequence
  Description of Artificial Sequence Synthetic polynucleotide
atggccatggccgccgccgccgtgatcgtgcccctgggcatcctgttcttcatctccggcctggtggtgaacctgctgcaggccgtgtgctacgtgctgatccgccccctgtccaagaacacctaccgcaagatcaaccgcgtggtggccgagaccctgtggctggagctggtgtggatcgtggactggtgggccggcgtgaagatccaggtgttcgccgacgacgagaccttcaaccgcatgggcaaggagcacgccctggtggtgtgcaaccaccgctccgacatcgactggctggtgggctggatcctggcccagcgctccggctgcctgggctccgccctggccgtgatgaagaagtcctccaagttcctgcccgtgatcggctggtccatgtggttctccgagtacctgttcctggagcgcaactgggccaaggacgagtccaccctgaagtccggcctgcagcgcctgaacgacttcccccgccccttctggctggccctgttcgtggagggcacccgcttcaccgaggccaagctgaaggccgcccaggagtacgccgcctcctcccagctgcccgtgccccgcaacgtgctgatcccccgcaccaagggcttcgtgtccgccgtgtccaacatgcgctccttcgtgcccgccatctacgacatgaccgtggccatccccaagacctcccccccccccaccatgctgcgcctgttcaagggccagccctccgtggtgcacgtgcacatcaagtgccactccatgaaggacctgcccgagtccgacgacgccatcgcccagtggtgccgcgaccagttcgtggccaaggacgccctgctggacaagcacatcgccgccgacaccttccccggccagaaggagcacaacatcggccgccccatcaagtccctggccgtggtggtgtcctgggcctgcctgctgaccctgggcgccatgaagttcctgcactggtccaacctgttctcctccctgaagggcatcgccctgtccgccctgggcctgggcatcatcaccctgtgcatgcagatcctgatccgctcctcccagtccgagcgctccacccccgccaaggtggcccccgccaagcccaaggacaagcaccagtccggctcctcctcccagaccgaggtggaggagaagcagaagtga

SEQ ID NO: 90 - DNA, 1164 bp, Artificial Sequence
  Description of Artificial Sequence Synthetic polynucleotide
atggccatcgccgccgccgccgtgatcgtgcccctgggcctgctgttcttcatctccggcctggtggtgaacctgatccaggccctgtgcttcgtgctgatccgccccctgtccaagaacacctaccgcaagatcaaccgcgtggtggccgagctgctgtggctggagctgatctggctggtggactggtgggccggcgtgaagatcaaggtgttcatggaccccgagtccttcaacctgatgggcaaggagcacgccctggtggtggccaaccaccgctccgacatcgactggctggtgggctggctgctggcccagcgctccggctgcctgggctccgccctggccgtgatgaagaagtcctccaagttcctgcccgtgatcggctggtccatgtggttctccgagtacctgttcctggagcgctcctgggccaaggacgagaacaccctgaaggccggcctgcagcgcctgaaggacttcccccgccccttctggctggccttcttcgtggagggcacccgcttcacccaggccaagttcctggccgcccaggagtacgccgcctcccagggcctgcccatcccccgcaacgtgctgatcccccgcaccaagggcttcgtgtccgccgtgtcccacatgcgctccttcgtgcccgccatctacgacatgaccgtggccatccccaagtcctccccctcccccaccatgctgcgcctgttcaagggccagccctccgtggtgcacgtgcacatcaagcgctgcctgatgaaggagctgcccgagaccgacgaggccgtggcccagtggtgcaaggacatgttcgtggagaaggacaagctgctggacaagcacatcgccgaggacaccttctccgaccagcccatgcaggacctgggccgccccatcaagtccctgctggtggtggcctcctgggcctgcctgatggcctacggcgccctgaagttcctgcagtgctcctccctgctgtcctcctggaagggcatcgccttcttcctggtgggcctggccatcgtgaccatcctgatgcacatcctgatcctgttctcccagtccgagcgctccacccccgccaaggtggcccccggcaagcccaagaacgacggcgagacctccgaggcccgccgcgacaagcagcagtga

SEQ ID NO: 91 - DNA, 1164 bp, Artificial Sequence
  Description of Artificial Sequence Synthetic polynucleotide
atggccatccccgccgccatcgtgatcgtgcccgtgggcctgctgttcttcatctccggcctgatcgtgaacctgctgcaggccctgtgcttcgtgctgatccgccccctgtccaagtccgcctaccgcaccatcaaccgccagctggtggagctgctgtggctggagctggtgtgcatcgtggactggtgggcccgcgtgaagatccagctgttcaccgacaaggagaccctgaactccatgggcaaggagcacgccctggtgatgtgcaaccaccgctccgacatcgactggctggtgggctggatcctggcccagcgctccggctgcctgggctccaccgtggccgtgatgaagaagtcctccaaggtgctgcccgtgatcggctggtccatgtggttctccgagtacctgttcctggagcgcaactgggccaaggacgagtccaccctgaagtccggcctgcagcgcctgcgcgacttcccccgccccttctggctggccctgttcgtggagggcacccgcttcacccagcccaagctgctggccgcccaggagtacgccgcctccaccggcctgcccatcccccgcaacgtgctgatcccccgcaccaagggcttcgtgtccgccgtgtccatcacccgctccttcgtgcccgtgatctacgacatcaccgtggccatccccaagtcctccccccagcccaccatgctgcgcctgttcaagggccagtcctccgtggtgcacgtgcacctgaagcgccacctgatgaaggacctgcccgagtccgacgacgacgtggcccagtggtgccgcgaccagttcgtggtgaaggactccctgctggacaagcacatcgccgaggacaccttctccgaccaggagctgcaggacatcggccgccccatcaagtccctggtggtgttcacctcctgggtgtgcatcatcaccttcggcgccctgaagttcctgcagtggtcctccctgctgcactcctggaagggcatcgccatctccgcctccggcctggccatcgtgaccgtgctgatgcacatcctgatccgcttctcccagtccgagcactccacctccgccaagatcgccgccgagaagcacaagaacggcggcgtgtcccaggagatgggccgcgagaagcagcactga

SEQ ID NO: 92 - DNA, 1164 bp, Artificial Sequence
  Description of Artificial Sequence Synthetic polynucleotide
atggagatccccgccgtggccgtgatcgtgcccatcggcatcctgttcttcatctccggcctgatcgtgaacctgatgcaggccatctgcttcttcctgatccgccccctgtccaagaacacccaccgcatcgtgaaccgccagctggccgagctgctgtggctggagctgatctggatcgtggactggtgggccggcgtgaagatccagctgttcaccgacaaggagaccctgcacctgatgggcaaggagcacgccctggtgatctgcaaccactcctccgacatcgactggctggtgggctggctgctgtgccagcgctccggctgcctgggctccgccctggccgtgatgaagtcctcctccaaggtgctgcccgtgatcggctggtccatgtggttctccgagtacctgttcctggagcgctcctgggccaaggacgagtccaccctgaagtccggcctgcagcgcctgaaggacttcccccgccccttctggctggccctgttcgtggagggcacccgcttcacccaggccaagctgctggccgcccaggagtacgccatgtccgccggcctgcccgtgccccgcaacgtgctgatcccccgcaccaagggcttcgtgtccgccgtgtccaacatgcgctccttcgtgcccgccatctacgacgtgaccgtggccatccccaagtcctccgtgcagcccaccatgctgcgcctgttcaagggccagtcctccgtggtgcaggtgcacctgaagcgccactccatgaaggacctgcccgagtccgaggacgacgtggcccagtggtgccgcgaccgcttcgtggtgaaggactccctgctggacaagcacaaggtggaggacaccttcaccgaccaggagctgcaggacctgggccgccccatcaagtccctggtggtggtgacctgctgggcctgcatcatcatcttcggcatcctgaagttcctgcagtggtcctccctgctgtactcctggaagggcatggccatctccgcctccggcctggccgtggtgaccttcctgatgcagatcctgatccgcttctcccagtccgagcgctccacccccgccaagatcgcccccgccaagcccaacaaggccggcaactcctccgagaccgtgcgcgacaagcaccagtga

SEQ ID NO: 93 - DNA, 1137 bp, Artificial Sequence
  Description of Artificial Sequence Synthetic polynucleotide
atggccatccccgccgccatcatcatcgtgcccctgggcctgatcttcttcacctccggcctgatcatcaacctgatccaggccgtgtgctacgtgctgatccgccccctgtccaagtccaccttccgccgcatcaaccgcgagctggccgagctgctgtggctggagctggtgtgggtggtggactggtgggccggcgtgaagatccagctgttcaccgacaaggagaccctgcactccatgggcaaggagcacgccctggtgatctgcaaccaccgctccgacatcgactggctggtgggctggatcctggcccagcgctccggctgcctgggctccgccctggccgtgatgaagaagtcctccaaggtgctgcccgtgatcggctggtccatgtggttctccgagtacttcttcctggagcgcaactgggccatggacgagtccaccctgaagtccggcctgcagcgcctgaaggacttcccccagcccttctggctggccctgttcgtggagggcacccgcttcacccagcccaagctgctggccgcccaggagtacgccgcctccgccggcctgcccatcccccgcaacgtgctgatcccccgcaccaagggcttcgtgtccgccgtgaacatcatgcgctccttcgtgcccgccatctacgacgtgaccgtggccatccccaagtcctccccccagcccaccatgctgcgcctgttcaagggccagtcctccgtggtgcacgtgcacctgaagcgccacctgatggaggacctgcccgagaccgacgacgacgtggcccagtggtgccgcgaccgcttcgtggtgaaggactccctgctggacaagtacgtggccgaggacaccttctccgaccaggagctgcaggacctgggccgccccatcaagtccctggtggtggtgacctcctgggtgtgcatcatcgccttcggctccctgaagttcctgcagtggtcctccctgctgtactcctggaagggcatcgtgatctccgccgcctccctggccgtggtgaccgtgctgatgcagatcctgatccgcttctcccagtccgagcgctccacctccgccaagatcgccgccgccaagcgcaagaacgtgggcgagcactga

SEQ ID NO: 94 - DNA, 1164 bp, Artificial Sequence
  Description of Artificial Sequence Synthetic polynucleotide
atggccatccccgtggtggtggtgatcgtgcccgtgggcctgctgttcttcatctccggcctgatcgtgaacctgctgcaggccctgtgcttcgtgctgatccgccccctgtccaagtccgcctaccgcaccatcaaccgccagctggtggagctgctgtggctggagctggtgtgcatcgtggactggtgggcccgcgtgaagatccagctgttcatcgacaaggagaccctgaactccatgggcaaggagcacgccctggtgatgtgcaaccaccgctcctacatcgactggctggtgggctggatcctggcccagcgctccggctgcctgggctccaccgtggccgtgatgaagaagtcctccaaggtgctgcccgtgatcggctggtccatgtggttctccgagtacctgttcctggagcgcaactgggccaaggacgagtccaccctgaagtccggcctgcagcgcctgcgcgacttcccccgccccttctggctggccctgttcgtggagggcacccgcttcacccagcccaagctgctggccgcccaggagtacgccgcctccaccggcctgcccatcccccgcaacgtgctgatcccccgcaccaagggcttcgtgtccgccgtgtccatcacccgctccttcgtgcccgtgatctacgacatcaccgtggccatccccaagtcctcctcccagcccaccatgctgaagctgttcaagggccagtcctccgtggtgcacgtgcacctgaagcgccacctgatgaaggacctgcccgagtccgacgacgacgtggcccagtggtgccgcgcccagttcgtggtgaaggactccctgctggacaagcacatcgccgaggacaccttctccgaccaggagctgcaggacatcggccgccccatcaagtccctggtggtgttcacctcctgggtgtgcatcatcaccttcggcgccctgaagttcctgcagtggtcctccctgctgcactcctggaagggcatcgccatctccgcctccggcctggccatcgtgaccgtgctgatgcacatcctgatccgcttctcccagtccgagcactccacctccgccaagatcgccgccgagaagcacaagaacggcggcgtgtcccaggagatgggccgcgagaagcagcactga

SEQ ID NO: 95 - DNA, 1164 bp, Artificial Sequence
  Description of Artificial Sequence Synthetic polynucleotide
atgggcatccccgccgtggccgtgatcgtgcccatcggcatcctgttcttcatctccggcttcatcgtgaacctgatgcaggccatctgcttcgtgctgatccgccccctgtccaagaacacctaccgcatcgtgaaccgccagctggccgagttcctgtggctggagctgatctgggtggtggactggtgggccggcgtgaagatccagctgttcaccgacaaggagaccctgcacctgatgggcaaggagcacgccctggtgatctgcaaccaccgctccgacatcgactggctggtgggctggctgctgtgccagcgctccggctgcctgggctccgccctggccgtgatgaagtcctcctccaaggtgctgcccgtgatcggctggtccatgtggttctccgagtacctgttcctggagcgctcctgggccaaggacgagtccaccctgaagctgggcctgcagcgcctgaaggacttcccccgccccttctggctggccctgttcgtggagggcacccgcttcacccaggccaagctgctggccgcccaggagtacgccatgtccgccggcctgcccgtgccccgcaacgtgctgatcccccgcaccaagggcttcgtgtccgccgtgtccaacatgcgctccttcgtgcccgccatctacgacgtgaccgtggccatccccaagtcctccgtgcagcccaccatgctgggcctgttcaagggccagtcctgcgtggtgcaggtgcacctgaagcgccacctgatgaaggacctgcccgagtccgaggacgacgtggcccagtggtgccgcgagcgcttcgtggtgaaggactccctgctggacaagcacaaggtggaggacaccttctccgaccaggagctgcaggacctgggccgccccatcaagtccctggtggtggtgatctcctgggcctgcatcctgatcttctggatcctgaagttcctgcagtggtcctccctgctgtactcctggaagggcatcgccatctccgcctgcgccatggccgtgatcgccttcctgatgcagatcctgctgcgcttctcccagtccgagcgctccacccccgccaagatcgcccccgccaagcccaacaacgcccgcaactcctccgagaccgtgcgcgacaagcaccagtga

SEQ ID NO: 96 - DNA, 1137 bp, Artificial Sequence
  Description of Artificial Sequence Synthetic polynucleotide
atggccatccccgccgccatcatcatcgtgcccctgggcctgatcttcttcacctccggcttcatcatcaacctgatccaggccgtgtgctacgtgctgatccgccccctgtccaagtccaccttccgccgcatcaaccgccagctggccgagctgctgtggctggagctggtgtgggtggtggactggtgggccggcgtgaagatccagctgttcaccaacaaggagaccctgcactccatcggcaaggagcacgccctggtgatctgcaaccagcgctccgacatcgactggctggtgggctggatcctggcccagcgctccggctgcctgggctccgccctggccgtgatgaagaagtcctccaaggtgctgcccgtgatcggctggtccatgtggttctccgagtacctgttcctggagcgcaactgggccatggacgagtccaccctgaagtccggcctgcagtggctgaaggacttcccccagcccttctggctggccctgttcgtggagggcacccgcttcacccagcccaagctgctggccgcccaggagtacgccgcctccgccggcctgcccatcccccgcaacgtgctgatcccccgcaccaagggcttcgtgtccgccgtgaacatcatgcgctccttcgtgcccgccgtgtacgacgtgaccgtggccatccccaagtcctccccccagcccaccatgctgcgcctgttcaagggccagtcctccgtggtgcacgtgcacctgaagcgccacctgatggaggacctgcccgagaccgacgacgacgtggcccagtggtgccgcgaccgcttcgtggtgaaggactccctgctggacaagcacctggccgaggacaccttctccgaccaggagctgcaggacctgggccgccccatcaagtccctggtggtggtgacctcctgggtgtgcatcatcgccttcggcgccctgaagttcctgcagtggtcctccctgctgtactcctggaagggcatcgtgatctccgccgcctccctggccgtggtgaccgtgctgatgcagatcctgatccgcttctcccagtccgagcgctccacctccgccaaggtggtggccgagaagcgcaagaacgtgggcgagcactga

SEQ ID NO: 97 - DNA, 5674 bp, Artificial Sequence
  Description of Artificial Sequence Synthetic polynucleotide
gtttaaacgccggtcaccacccgcatgctcgtactacagcgcacgcaccgcttcgtgatccaccgggtgaacgtagtcctcgacggaaacatctggttcgggcctcctgcttgcactcccgcccatgccgacaacctttctgctgttaccacgacccacaatgcaacgcgacacgaccgtgtgggactgatcggttcactgcacctgcatgcaattgtcacaagcgcttactccaattgtattcgtttgttttctgggagcagttgctcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaggtaccatggacgcctccggcgcctcctccttcctgcgcggccgctgcctggagtcctgcttcaaggcctccttcggctacgtaatgtcccagcccaaggacgccgccggccagccctcccgccgccccgccgacgccgacgacttcgtggacgacgaccgctggatcaccgtgatcctgtccgtggtgcgcatcgccgcctgcttcctgtccatgatggtgaccaccatcgtgtggaacatgatcatgctgatcctgctgccctggccctacgcccgcatccgccagggcaacctgtacggccacgtgaccggccgcatgctgatgtggattctgggcaaccccatcaccatcgagggctccgagttctccaacacccgcgccatctacatctgcaaccacgcctccctggtggacatcttcctgatcatgtggctgatccccaagggcaccgtgaccatcgccaagaaggagatcatctggtatcccctgttcggccagctgtacgtgctggccaaccaccagcgcatcgaccgctccaacccctccgccgccatcgagtccatcaaggaggtggcccgcgccgtggtgaagaagaacctgtccctgatcatcttccccgagggcacccgctccaagaccggccgcctgctgcccttcaagaagggcttcatccacatcgccctccagacccgcctgcccatcgtgccgatggtgctgaccggcacccacctggcctggcgcaagaactccctgcgcgtgcgccccgcccccatcaccgtgaagtacttctcccccatcaagaccgacgactgggaggaggagaagatcaaccactacgtggagatgatccacgccctgtacgtggaccacctgcccgagtcccagaagcccctggtgtccaagggccgcgacgcctccggccgctccaactcctgattaattaactcgagatgtggagatgtagggtggtcgactcgttggaggtgggtgtttttttttatcgagtgcgcggcgcggcaaacgggtccctttttatcgaggtgttcccaacgccgcaccgccctcttaaaacaacccccaccaccacttgtcgaccttctcgtttgttatccgccacggcgccccggaggggcgtcgtctggccgcgcgggcagctgtatcgccgcgctcgctccaatggtgtgtaatcttggaaagataataatcgatggatgaggaggagagcgtgggagatcagagcaaggaatatacagttggcacgaagcagcagcgtactaagctgtagcgtgttaagaaagaaaaactcgctgttaggctgtattaatcaaggagcgtatcaataattaccgaccctatacctttatctccaacccaatcgcggcctaggtgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatgactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcagatatcaagctccatcgagctccagccacggcaacaccgcgcgccttgcggccgagcacggcgacaagaacctgagcaagatctgcgggctgatcgccagcgacgagggccggcacgagatcgcctacacgcgcatcgtggacgagttcttccgcctcgaccccgagggcgccgtcgccgcctacgccaacatgatgcgcaagcagatcaccatgcccgcgcacctcatggacgacatgggccacggcgaggccaacccgggccgcaacctcttcgccgacttctccgcggtcgccgagaagatcgacgtctacgacgccgaggactactgccgcatcctggagcacctcaacgcgcgctggaaggtggacgagcgccaggtcagcggccaggccgccgcggaccaggagtacgtcctgggcctgccccagcgcttccggaaactcgccgagaagaccgccgccaagcgcaagcgcgtcgcgcgcaggcccgtcgccttctcctggatctccgggcgcgagatcatggtctagggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactcctgaagagcgtttaaac

SEQ ID NO: 98 - DNA, 1170 bp, Artificial Sequence
  Description of Artificial Sequence Synthetic polynucleotide
ggtaccatggccatccccgccgccgccgtgatcttcctgttcggcctgctgttcttcacctccggcctgatcatcaacctgttccaggccctgtgcttcgtgctggtgtggcccctgtccaagaacgcctaccgccgcatcaaccgcgtgttcgccgagctgctgctgtccgagctgctgtgcctgttcgactggtgggccggcgccaagctgaagctgttcaccgaccccgagaccttccgcctgatgggcaaggagcacgccctggtgatcatcaaccacatgaccgagctggactggatgctgggctgggtgatgggccagcacctgggctgcctgggctccatcctgtccgtggccaagaagtccaccaagttcctgcccgtgctgggctggtccatgtggttctccgagtacctgtacatcgagcgctcctgggccaaggaccgcaccaccctgaagtcccacatcgagcgcctgaccgactaccccctgcccttctggatggtgatcttcgtggagggcacccgcttcacccgcaccaagctgctggccgcccagcagtacgccgcctcctccggcctgcccgtgccccgcaacgtgctgatcccccgcaccaagggcttcgtgtcctgcgtgtcccacatgcgctccttcgtgcccgccgtgtacgacgtgaccgtggccttccccaagacctcccccccccccaccctgctgaacctgttcgagggccagtccatcgtgctgcacgtgcacatcaagcgccacgccatgaaggacctgcccgagtccgacgacgccgtggcccagtggtgccgcgacaagttcgtggagaaggacgccctgctggacaagcacaacgccgaggacaccttctccggccaggaggtgcaccgcaccggctcccgccccatcaagtccctgctggtggtgatctcctgggtggtggtgatcaccttcggcgccctgaagttcctgcagtggtcctcctggaagggcaaggccttctccgtgatcggcctgggcatcgtgaccctgctgatgcacatgctgatcctgtcctcccaggccgagcgctcctccaaccccgccaaggtggcccaggccaagctgaagaccgagctgtccatctccaagaaggccaccgacaaggagaactgactcgag

SEQ ID NO: 99 - DNA, 1167 bp, Artificial Sequence
  Description of Artificial Sequence Synthetic polynucleotide
ggtaccatggccatccccgccgccgccgtgatcttcctgttcggcctgatcttcttcgcctccggcctgatcatcaacctgttccaggccctgtgcttcgtgctgatctggcccatctccaagaacgcctaccgccgcatcaaccgcgtgttcgccgagctgctgctgtccgagctgctgtgcctgttcgactggtgggccggcgccaagctgaagctgttcaccgaccccgagaccttccgcctgatgggcaaggagcacgccctggtgatcatcaaccacatgaccgagctggactggatggtgggctgggtgatgggccagcacttcggctgcctgggctccatcctgtccgtggccaagaagtccaccaagttcctgcccgtgctgggctggtccatgtggttcaccgagtacctgtacatcgagcgctcctggaacaaggacaagtccaccctgaagtcccacatcgagcgcctgaaggactaccccctgcccttctggctggtgatcttcgccgagggcacccgcttcacccagaccaagctgctggccgcccagcagtacgccgcctcctccggcctgcccgtgccccgcaacgtgctgatcccccgcaccaagggcttcgtgtcctgcgtgtcccacatgcgctccttcgtgcccgccgtgtacgacctgaccgtggccttccccaagacctcccccccccccaccctgctgaacctgttcgagggccagtccgtggtgctgcacgtgcacatcaagcgccacgccatgaaggacctgcccgagtccgacgacgaggtggcccagtggtgccgcgacaagttcgtggagaaggacgccctgctggacaagcacaacgccgaggacaccttctccggccaggagctgcagcacaccggccgccgccccatcaagtccctgctggtggtgatctcctgggtggtggtgatcgccttcggcgccctgaagttcctgcagtggtcctcctggaagggcaaggccttctccgtgatcggcctgggcatcgtgaccctgctgatgcacatgctgatcctgtcctcccaggccgagcgctccaagcccgccaaggtggcccaggccaagctgaagaccgagctgtccatctccaagaccgtgaccgacaaggagaactgactcgag

SEQ ID NO: 100 - DNA, 1188 bp, Artificial Sequence
  Description of Artificial Sequence Synthetic polynucleotide
ggtaccatggccatcccctccgccgccgtggtgttcctgttcggcctgctgttcttcacctccggcctgatcatcaacctgttccaggccttctgcttcgtgctgatctcccccctgtccaagaacgcctaccgccgcatcaaccgcgtgttcgccgagctgctgcccctggagttcctgtggctgttccactggtgcgccggcgccaagctgaagctgttcaccgaccccgagaccttccgcctgatgggcaaggagcacgccctggtgatcatcaaccacaagatcgagctggactggatggtgggctgggtgctgggccagcacctgggctgcctgggctccatcctgtccgtggccaagaagtccaccaagttcctgcccgtgttcggctggtccctgtggttctccggctacctgttcctggagcgctcctgggccaaggacaagatcaccctgaagtcccacatcgagtccctgaaggactaccccctgcccttctggctgatcatcttcgtggagggcacccgcttcacccgcaccaagctgctggccgcccagcagtacgccgcctcctccggcctgcccgtgccccgcaacgtgctgatcccccacaccaagggcttcgtgtcctccgtgtcccacatgcgctccttcgtgcccgccatctacgacgtgaccgtggccttccccaagacctcccccccccccaccatgctgaagctgttcgagggccagtccgtggagctgcacgtgcacatcaagcgccacgccatgaaggacctgcccgagtccgacgacgccgtggcccagtggtgccgcgacaagttcgtggagaaggacgccctgctggacaagcacaactccgaggacaccttctccggccaggaggtgcaccacgtgggccgccccatcaaggccctgctggtggtgatctcctgggtggtggtgatcatcttcggcgccctgaagttcctgctgtggtcctccctgctgtcctcctggaagggcaaggccttctccgtgatcggcctgggcatcgtggccggcatcgtgaccctgctgatgcacatcctgatcctgtcctcccaggccgagggctccaaccccgtgaaggccgcccccgccaagctgaagaccgagctgtcctcctccaagaaggtgaccaacaaggagaactgactcgag

SEQ ID NO: 101 - DNA, 1188 bp, Artificial Sequence
  Description of Artificial Sequence Synthetic polynucleotide
ggtaccatggccatcccctccgccgccgtggtgttcctgttcggcctgctgttcttcacctccggcctgatcatcaacctgttccaggccttctgcttcgtgctgatctcccccctgtccaagaacgcctaccgccgcatcaaccgcgtgttcgccgagctgctgcccctggagttcctgtggctgttccactggtgcgccggcgccaagctgaagctgttcaccgaccccgagaccttccgcctgatgggcaaggagcacgccctggtgatcatcaaccacaagatcgagctggactggatggtgggctgggtgctgggccagcacctgggctgcctgggctccatcctgtccgtggccaagaagtccaccaagttcctgcccgtgttcggctggtccctgtggttctccgagtacctgttcctggagcgctcctgggccaaggacaagatcaccctgaagtcccacatcgagtccctgaaggactaccccctgcccttctggctgatcatcttcgtggagggcacccgcttcacccgcaccaagctgctggccgcccagcagtacgccgcctcctccggcctgcccgtgccccgcaacgtgctgatcccccacaccaagggcttcgtgtcctccgtgtcccacatgcgctccttcgtgcccgccatctacgacgtgaccgtggccttccccaagacctcccccccccccaccatgctgaagctgttcgagggccagtccgtggagctgcacgtgcacatcaagcgccacgccatgaaggacctgcccgagtccgacgacgccgtggcccagtggtgccgcgacaagttcgtggagaaggacgccctgctggacaagcacaactccgaggacaccttctccggccaggaggtgcaccacgtgggccgccccatcaaggccctgctggtggtgatctcctgggtggtggtgatcatcttcggcgccctgaagttcctgctgtggtcctccctgctgtcctcctggaagggcaaggccttctccgtgatcggcctgggcatcgtggccggcatcgtgaccctgctgatgcacatcctgatcctgtcctcccaggccgagggctccaaccccgtgaaggccgcccccgccaagctgaagaccgagctgtcctcctccaagaaggtgaccaacaaggagaactgactcgag

SEQ ID NO: 102 - DNA, 1122 bp, Artificial Sequence
  Description of Artificial Sequence Synthetic polynucleotide
ggtaccatggccatcgccgccgccgccgtgatcttcctgttcggcctgctgttcttcgcctccggcatcatcatcaacctgttccaggccctgtgcttcgtgctgatctggcccctgtccaagaacgtgtaccgccgcatcaaccgcgtgttcgccgagctgctgctgatggacctgctgtgcctgttccactggtgggccggcgccaagatcaagctgttcaccgaccccgagaccttccgcctgatgggcatggagcacgccctggtgatcatgaaccacaagaccgacctggactggatggtgggctggatcctgggccagcacctgggctgcctgggctccatcctgtccatcgccaagaagtccaccaagttcatccccgtgctgggctggtccgtgtggttctccgagtacctgttcctggagcgctcctgggccaaggacaagtccaccctgaagtcccacatggagaagctgaaggactaccccctgcccttctggctggtgatcttcgtggagggcacccgcttcacccgcaccaagctgctggccgcccagcagtacgccgcctcctccggcctgcccgtgccccgcaacgtgctgatcccccacaccaagggcttcgtgtcctgcgtgtccaacatgcgctccttcgtgcccgccgtgtacgacgtgaccgtggccttccccaagtcctcccccccccccaccatgctgaagctgttcgagggccagtccatcgtgctgcacgtgcacatcaagcgccacgccctgaaggacctgcccgagtccgacgacgccgtggcccagtggtgccgcgacaagttcgtggagaaggacgccctgctggacaagcacaacgccgaggacaccttctccggccaggaggtgcaccacatcggccgccccatcaagtccctgctggtggtgatcgcctgggtggtggtgatcatcttcggcgccctgaagttcctgcagtggtcctccctgctgtccacctggaagggcaaggccttctccgtgatcggcctgggcatcgccaccctgctgatgcacatgctgatcctgtcctcccaggccgagcgctccaaccccgccaaggtggccaagtgactcgag

SEQ ID NO: 103 - DNA, 1176 bp, Artificial Sequence
  Description of Artificial Sequence Synthetic polynucleotide
ggtaccatgaccatcgcctccgccgccgtggtgttcctgttcggcatcctgctgttcacctccggcctgatcatcaacctgttccaggccttctgctccgtgctggtgtggcccctgtccaagaacgcctaccgccgcatcaaccgcgtgttcgccgagttcctgcccctggagttcctgtggctgttccactggtgggccggcgccaagctgaagctgttcaccgaccccgagaccttccgcctgatgggcaaggagcacgccctggtgatcatcaaccacaagatcgagctggactggatggtgggctgggtgctgggccagcacctgggctgcctgggctccatcctgtccgtggccaagaagtccaccaagttcctgcccgtgttcggctggtccctgtggttctccgagtacctgttcctggagcgcaactgggccaaggacaagaagaccctgaagtcccacatcgagcgcctgaaggactaccccctgcccttctggctgatcatcttcgtggagggcacccgcttcacccgcaccaagctgctggccgcccagcagtacgccgcctccgccggcctgcccgtgccccgcaacgtgctgatcccccacaccaagggcttcgtgtcctccgtgtcccacatgcgctccttcgtgcccgccatctacgacgtgaccgtggccttccccaagacctcccccccccccaccatgctgaagctgttcgagggccacttcgtggagctgcacgtgcacatcaagcgccacgccatgaaggacctgcccgagtccgaggacgccgtggcccagtggtgccgcgacaagttcgtggagaaggacgccctgctggacaagcacaacgccgaggacaccttctccggccaggaggtgcaccacgtgggccgccccatcaagtccctgctggtggtgatctcctgggtggtggtgatcatcttcggcgccctgaagttcctgcagtggtcctccctgctgtcctcctggaagggcatcgccttctccgtgatcggcctgggcaccgtggccctgctgatgcagatcctgatcctgtcctcccaggccgagcgctccatccccgccaaggagacccccgccaacctgaagaccgagctgtcctcctccaagaaggtgaccaacaaggagaactgactcgag

SEQ ID NO: 104 - DNA, 1176 bp, Artificial Sequence
  Description of Artificial Sequence Synthetic polynucleotide
ggtaccatggccatcgccgccgccgccgtgatcgtgcccgtgtccctgctgttcttcgtgtccggcctgatcgtgaacctggtgcaggccgtgtgcttcgtgctgatccgccccctgttcaagaacacctaccgccgcatcaaccgcgtggtggccgagctgctgtggctggagctggtgtggctgatcgactggtgggccggcgtgaagatcaaggtgttcaccgaccacgagaccttccacctgatgggcaaggagcacgccctggtgatctgcaaccacaagtccgacatcgactggctggtgggctgggtgctggcccagcgctccggctgcctgggctccaccctggccgtgatgaagaagtcctccaagttcctgcccgtgatcggctggtccatgtggttctccgagtacctgttcctggagcgcaactgggccaaggacgagtccaccctgaagtccggcctgaaccgcctgaaggactaccccctgcccttctggctggccctgttcgtggagggcacccgcttcacccgcgccaagctgctggccgcccagcagtacgccgcctcctccggcctgcccgtgccccgcaacgtgctgatcccccgcaccaagggcttcgtgtcctccgtgtcccacatgcgctccttcgtgcccgccatctacgacgtgaccgtggccatccccaagacctcccccccccccaccctgctgcgcatgttcaagggccagtcctccgtgctgcacgtgcacctgaagcgccaccagatgaacgacctgcccgagtccgacgacgccgtggcccagtggtgccgcgacatcttcgtggagaaggacgccctgctggacaagcacaacgccgaggacaccttctccggccaggagctgcaggacaccggccgccccatcaagtccctgctgatcgtgatctcctgggccgtgctggtggtgttcggcgccgtgaagttcctgcagtggtcctccctgctgtcctcctggaagggcctggccttctccggcatcggcctgggcgtgatcaccctgctgatgcacatcctgatcctgttctcccagtccgagcgctccacccccgccaaggtggcccccgccaagcccaagatcgagggcgagtcctccaagaccgagatggagaaggagcactgactcgag

SEQ ID NO: 105 - DNA, 1176 bp, Artificial Sequence
  Description of Artificial Sequence Synthetic polynucleotide
ggtaccatggccatcgccgccgccgccgtgatcgtgcccctgggcctgctgttcttcgtgtccggcctgatcgtgaacctggtgcaggccgtgtgcttcgtgctgatccgccccctgtccaagaacacctaccgccgcatcaaccgcgtggtggccgagctgctgtggctggagctggtgtggctgatcgactggtgggccggcgtgaagatcaaggtgttcaccgaccacgagaccctgtccctgatgggcaaggagcacgccctggtgatctgcaaccacaagtccgacatcgactggctggtgggctgggtgctggcccagcgctccggctgcctgggctccaccctggccgtgatgaagaagtcctccaagttcctgcccgtgatcggctggtccatgtggttctccgagtacctgcccgagtccgacgacgccgtggcccagtggtgccgcgacatcttcgtggagaaggacgccctgctggacaagcacaacgccgaggacaccttctccggccaggagctgcaggacaccggccgccccatcaagtccctgctggtggtgatctcctgggccgtgctggtgatcttcggcgccgtgaagttcctgcagtggtcctccctgctgtcctcctggaagggcctggccttctccggcgtgggcctgggcatcatcaccctgctgatgcacatcctgatcctgttctcccagtccgagcgctccacccccgccaaggtggcccccgccaagcccaagaaggacggcgagtcctccaagaccgagatcgagaaggagaacgttcctggagcgctcctgggccaaggacgagaacaccctgaagtccggcctgaaccgcctgaaggactaccccctgcccttctggctggccctgttcgtggagggcacccgcttcacccgcgccaagctgctggccgcccagcagtacgccacctcctccggcctgcccgtgccccgcaacgtgctgatcccccgcaccaagggcttcgtgtcctccgtgtcccacatgcgctccttcgtgcccgccatctacgacgtgaccgtggccatccccaagacctcccccccccccaccatgctgcgcatgttcaagggccagtcctccgtgctgcacgtgcacctgaagcgccacctgatgaaggaccttgactcgag

SEQ ID NO: 106 - DNA, 1167 bp, Artificial Sequence
  Description of Artificial Sequence Synthetic polynucleotide
ggtaccatggccatcgccgccgccgccgtgatcttcctgttcggcctgatcttcttcgcctccggcctgatcatcaacctgttccaggccctgtgcttcgtgctgatccgccccctgtccaagaacgcctaccgccgcatcaaccgcgtgttcgccgagctgctgctgtccgagctgctgtgcctgttcgactggtgggccggcgccaagctgaagctgttcaccgaccccgagaccttccgcctgatgggcaaggagcacgccctggtgatcatcaaccacatgaccgagctggactggatggtgggctgggtgatgggccagcacttcggctgcctgggctccatcatctccgtggccaagaagtccaccaagttcctgcccgtgctgggctggtccatgtggttctccgagtacctgtacctggagcgctcctgggccaaggacaagtccaccctgaagtcccacatcgagcgcctgatcgactaccccctgcccttctggctggtgatcttcgtggagggcacccgcttcacccgcaccaagctgctggccgcccagcagtacgccgtgtcctccggcctgcccgtgccccgcaacgtgctgatcccccgcaccaagggcttcgtgtcctgcgtgtcccacatgcgctccttcgtgcccgccgtgtacgacgtgaccgtggccttccccaagacctcccccccccccaccctgctgaacctgttcgagggccagtccatcatgctgcacgtgcacatcaagcgccacgccatgaaggacctgcccgagtccgacgacgccgtggccgagtggtgccgcgacaagttcgtggagaaggacgccctgctggacaagcacaacgccgaggacaccttctccggccaggaggtgtgccactccggctcccgccagctgaagtccctgctggtggtgatctcctgggtggtggtgaccaccttcggcgccctgaagttcctgcagtggtcctcctggaagggcaaggccttctccgccatcggcctgggcatcgtgaccctgctgatgcacgtgctgatcctgtcctcccaggccgagcgctccaaccccgccgaggtggcccaggccaagctgaagaccggcctgtccatctccaagaaggtgaccgacaaggagaactgactcgag

SEQ ID NO: 107 - DNA, 1155 bp, Artificial Sequence
  Description of Artificial Sequence Synthetic polynucleotide
ggtaccatggccatccccgccgccgtggccgtgatccccatcggcctgctgttcatcatctccggcctgatcgtgaacctgatccaggccgtggtgtacgtgctgatccgccccctgtccaagaacctgcaccgcaagatcaacaagcccatcgccgagctgctgtggctggagctgatctggctggtggactggtgggccggcatcaaggtggaggtgtacgccgactcccagaccctggagctgatgggcaaggagcacgccctgctgatctgcaaccaccgctccgacatcgactggctggtgggctgggtgctggcccagcgcgcccgctgcctgggctccgccctggccatcatgaagaagtccgccaagttcctgcccgtgatcggctggtccatgtggttctccgactacatcttcctggaccgcacctgggccaaggacgagaagaccctgaagtccggcttcgagcgcctggccgacttccccatgcccttctggctggccctgttcgtggagggcacccgcttcaccaaggccaagctgctggccgcccaggagtacgccgcctcccgcggcctgcccgtgccccagaacgtgctgatcccccgcaccaagggcttcgtgaccgccgtgacccacatgcgctcctacgtgcccgccatctacgactgcaccgtggacatctccaaggcccaccccgccccctccatcctgcgcctgatccgcggccagtcctccgtggtgaaggtgcagatcacccgccactccatgcaggagctgcccgagaccgccgacggcatctcccagtggtgcatggacctgttcgtgaccaaggacggcttcctggagaagtaccactccaaggacatcttcggctccctgcccgtgcagaacatcggccgccccgtgaagtccctgatcgtggtgctgtgctggtactgcctgatggccttcggcctgttcaagttcttcatgtggtcctccctgctgtcctcctgggagggcatcctgtccctgggcctgatcctgctggccgtggccatcgtgatgcagatcctgatccagtccaccgagtccgagcgctccacccccgtgaagtccatccagaaggacccctccaaggagaccctgctgcagaactgactcgag

SEQ ID NO: 108 - DNA, 1161 bp, Artificial Sequence
  Description of Artificial Sequence Synthetic polynucleotide
ggtaccatgcacgtgctgctggagatggtgaccttccgcttctcctccttcttcgtgttcgacaacgtgcaggccctgtgcttcgtgctgatctggcccctgtccaagtccgcctaccgcaagatcaaccgcgtgttcgccgagctgctgctgtccgagctgctgtgcctgttcgactggtgggccggcgccaagctgaagctgttcaccgaccccgagaccttccgcctgatgggcaaggagcacgccctggtgatcaccaaccacaagatcgacctggactggatgatcggctggatcctgggccagcacttcggctgcctgggctccgtgatctccatcgccaagaagtccaccaagttcctgcccatcttcggctggtccctgtggttctccgagtacctgttcctggagcgcaactgggccaaggacaagcgcaccctgaagtcccacatcgagcgcatgaaggactaccccctgcccctgtggctgatcctgttcgtggagggcacccgcttcacccgcaccaagctgctggccgcccagcagtacgccgcctcctccggcctgcccgtgccccgcaacgtgctgatcccccacaccaagggcttcgtgtcctccgtgtcccacatgcgctccttcgtgcccgccgtgtacgacgtgaccgtggccttccccaagacctcccccccccccaccatgctgtccctgttcgagggccagtccgtggtgctgcacgtgcacatcaagcgccacgccatgaaggacctgcccgactccgacgacgccgtggcccagtggtgccgcgacaagttcgtggagaaggacgccctgctggacaagcacaacgccgaggacaccttctccggccaggaggtgcaccacgtgggccgccccatcaagtccctgctggtggtgatctcctggatggtggtgatcatcttcggcgccctgaagttcctgcagtggtcctccctgctgtcctcctggaagggcaaggccttctccgccatcggcctgggcatcgccaccctgctgatgcacgtgctggtggtgttctcccaggccgaccgctccaaccccgccaaggtgccccccgccaagctgaacaccgagctgtcctcctccaagaaggtgaccaacaaggagaactgactcgag

SEQ ID NO: 109 - DNA, 1155 bp, Artificial Sequence
  Description of Artificial Sequence Synthetic polynucleotide
ggtaccatggccatccccgccgccgtggccgtgatccccatcggcctgctgttcatcatctccggcctgatcgtgaacctgatccaggccgtggtgtacgtgctgatccgccccctgtccaagaacctgtaccgcaagatcaacaagcccatcgccgagctgctgtggctggagctgatctggctggtggactggtgggccggcatcaaggtggaggtgtacgccgactccgagaccctggagtccatgggcaaggagcacgccctgctgatctgcaaccaccgctccgacatcgactggctggtgggctgggtgctggcccagcgcgcccgctgcctgggctccgccctggccatcatgaagaagtccgccaagttcctgcccgtgatcggctggtccatgtggttctccgactacatcttcctggaccgcacctgggagaaggacgagaagaccctgaagtccggcttcgagcgcctggccgacttccccatgcccttctggctggccctgttcgtggagggcacccgcttcaccaaggccaagctgctggccgcccaggagttcgccgcctcccgcggcctgcccgtgccccagaacgtgctgatcccccgcaccaagggcttcgtgaccgccgtgacccacatgcgctcctacgtgcccgccatctacgactgcaccgtggacatctccaaggcccaccccgccccctccatcctgcgcctgatccgcggccagtcctccgtggtgaaggtgcagatcacccgccactccatgcaggagctgcccgagacccccgacggcatctcccagtggtgcatggacctgttcgtgaccaaggacgccttcctggagaagtaccactccaaggacatcttcggctccctgcccgtgcacgacatcggccgccccgtgaagtccctgatcgtggtgctgtgctggtactccctgatggccttcggcttctacaagttcttcatgtggtcctccctgctgtcctcctgggagggcatcctgtccctgggcctggtgctgatcgtgatcgccatcgtgatgcagatcctgatccagtcctccgagtccgagcgctccacccccgtgaagtccgtgcagaaggacccctccaaggagaccctgctgcagaactgactcgag

SEQ ID NO: 110 - DNA, 1137 bp, Artificial Sequence
  Description of Artificial Sequence Synthetic polynucleotide
ggtaccatggccaccggcggctccctgaagccctcctcctccgacctggacctggaccaccccaacatcgaggactacctgccctccggctcctccatcaacgagcccgccggcaagctgcgcctgcgcgacctgctggacatctcccccaccctgaccgaggccgccggcgccatcgtggacgactccttcacccgctgcttcaagtccatcccccgcgagccctggaactggaacctgtacctgttccccctgtggtgcatcggcgtgctgatccgctacttcatcctgttccccggccgcgtgatcgtgctgaccatgggctggatcaccgtgatctcctccttcatcgccgtgcgcgtgctgctgaagggccacgacgccctgcagatcaagctggagcgcctgatcgtgcagctgctgtgctcctccttcgtggcctcctggaccggcgtggtgaagtaccacggcccccgcccctccatccgccccaagcaggtgtacgtggccaaccacacctccatgatcgacttcttcatcctggaccagatgaccgtgttctccgtgatcatgcagaagcaccccggctgggtgggcctgctgcagtccaccctgctggagtccgtgggctgcatctggttcgaccgcgccgaggccaaggaccgcggcatcgtggccaagaagctgtgggaccacgtgcacggcgagggcaacaaccccctgctgatcttccccgagggcacctgcgtgaacaacaactactccgtgatgttcaagaagggcgccttcgagctgggctgcaccgtgtgccccgtggccatcaagtacaacaagatcttcgtggacgccttctggaactccaagaagcagtccttcacccgccacctgctgcagctgatgacctcctgggccgtggtgtgcgacgtgtggtacttggagccccagaccctgaagcccggcgagacccccatcgagttcgccgagcgcgtgcgcgacatcatctccgcccgcgccggcctgaagaaggtgccctgggacggctacctgaagtactcccgcccctcccccaagcaccgcgagcgcaagcagcagaccttcgccgagtccgtgctgcagcgcctggaggagtgactcgag

SEQ ID NO: 111 - DNA, 1140 bp, Artificial Sequence
  Description of Artificial Sequence Synthetic polynucleotide
ggtaccatggccaccgccggctccctgaagccctcccgctccgagctggacttcgaccgccccaacatcgaggactacctgccctccggctcctccatcatcgagcccgccggcaagctgcgcctgcgcgacctgctggacatctcccccaccctgaccgaggccgccggcgccatcgtggacgactccttcacccgctgcttcaagtccaacccccccgagccctggaactggaacatctacctgttccccctgtggtgcttcggcgtgctgatccgctacctgatcctgttccccgcccgcgtgatcgtgctgaccatcggctggatcatcttcctgtcctccttcatccccgtgcacctgctgctgaagggccacgacgccctgcgcatcaagctggagcgcctgctggtggagctgatctgctccttcttcgtggcctcctggaccggcgtggtgaagtaccacggcccccgcccctccatccgccccaagcaggtgtacgtggccaaccacacctccatgatcgacttcttcatcctggaccagatgaccgtgttctccgtgatcatgcagaagcaccccggctgggtgggcctgctgcagtccaccctgctggagtccgtgggctgcatctggttcgaccgcgccgaggccaaggaccgcggcatcgtggccaagaagctgtgggaccacgtgcacggcgagggcaacaaccccctgctgatcttccccgagggcacctgcgtgaacaacaactactccgtgatgttcaagaagggcgccttcgagctgggctgcaccgtgtgccccgtggccatcaagtacaacaagatcttcgtggacgccttctggaactccaagaagcagtccttcacccgccacctgctgcagctgatgacctcctgggccgtggtgtgcgacgtgtggtacttggagccccagaccctgaagcccggcgagacccccatcgagttcgccgagcgcgtgcgcgacatcatctccgtgcgcgccggcctgaagaaggtgccctgggacggctacctgaagtactcccgcccctcccccaagcacaccgagcgcaagcagcagaacttcgccgagtccgtgctgcagcgcctggagaagaagtgactcgag

SEQ ID NO: 112 - DNA, 1140 bp, Artificial Sequence
  Description of Artificial Sequence Synthetic polynucleotide
ggtaccatggccaccggcggccgcctgaagccctcctcctccgagctggacctggaccgcgccaacaccgaggactacctgccctccggctcctccatcaacgagcccgtgggcaagctgcgcctgcgcgacctgctggacatctcccccaccctgaccgaggccgccggcgccatcgtggacgactccttcacccgctgcttcaagtccatcccccccgagccctggaactggaacatctacctgttccccctgtggtgcttcggcgtgctgatccgctacttcatcctgttccccgcccgcgtgatcgtgctgaccatcggctggatcaccgtgatctcctccttcaccgccgtgcgcttcctgctgaagggccacaacgccctgcagatcaagctggagcgcctgatcgtgcagctgctgtgctcctccttcgtggcctcctggaccggcgtggtgaagtaccacggcccccgcccctccatccgccccaagcaggtgtacgtggccaaccacacctccatgatcgacttcctgatcctggaccagatgaccgtgttctccgtgatcatgcagaagcaccccggctgggtgggcctgctgcagtccaccctgctggagtccgtgggctgcatctggttcaaccgcgccgaggccaaggaccgcgagatcgtggccaagaagctgtgggaccacgtgcacggcgagggcaacaaccccctgctgatcttccccgagggcacctgcgtgaacaaccactactccgtgatgttcaagaagggcgccttcgagctgggctgcaccgtgtgccccgtggccatcaagtacaacaagatcttcgtggacgccttctggaactcccgcaagcagtccttcaccatgcacctgctgcagctgatgacctcctgggccgtggtgtgcgacgtgtggtacttggagccccagaccctgaagcccggcgagaccgccatcgagttcgccgagcgcgtgcgcgacatcatctccgtgcgcgccggcctgaagaaggtgccctgggacggctacctgaagtactcccgcccctcccccaagcaccgcgagtccaagcagcagtccttcgccgagtccgtgctgcgccgcctggaggagaagtgactcgag

SEQ ID NO: 113 - DNA, 1140 bp, Artificial Sequence
  Description of Artificial Sequence Synthetic polynucleotide
ggtaccatggccaccggcggccgcctgaagccctcctcctccgagctggacctggaccgcgccaacaccgaggactacctgccctccggctcctccatcaacgagcccgtgggcaagctgcgcctgcgcgacctgctggacatctcccccaccctgaccgaggccgccggcgccatcgtggacgactccttcacccgctgcttcaagtccatcccccccgagccctggaactggaacatctacctgttccccctgtggtgcttcggcgtgctgatccgctacttcatcctgttccccgcccgcgtgatcgtgctgaccatcggctggatcaccgtgatctcctccttcaccgccgtgcgcttcctgctgaagggccacaacgccctgcagatcaagctggagcgcctgatcgtgcagctgctgtgctcctccttcgtggcctcctggaccggcgtggtgaagtaccacggcccccgcccctccatccgccccaagcaggtgtacgtggccaaccacacctccatgatcgacttcctgatcctggaccagatgaccgtgttctccgtgatcatgcagaagcaccccggctgggtgggcctgctgcagtccaccctgctggagtccgtgggctgcatctggttcaaccgcgccgaggccaaggaccgcgagatcgtggccaagaagctgtgggaccacgtgcacggcgagggcaacaaccccctgctgatcttccccgagggcacctgcgtgaacaaccactactccgtgatgttcaagaagggcgccttcgagctgggctgcaccgtgtgccccgtggccatcaagtacaacaagatcttcgtggacgccttctggaactccaagaagcactccttcacccgccacctgctgcagctgatgacctcctgggccgtggtgtgcgacgtgtggtacttggagccccagaccctgaagcccggcgagacccccatcgagttcgccgagcgcgtgcgcgacatcatctccgtgcgcgccgacctgaagaaggtgccctgggacggctacctgaagtactcccgcccctcccccaagcaccgcgagcgcaagcagcagaagttcgccgagtccgtgctgcgccgcctggaggagaagtgactcgag

SEQ ID NO: 114 - DNA, 1146 bp, Artificial Sequence
  Description of Artificial Sequence Synthetic polynucleotide
ggtaccatggccaccgccggccgcctgaagccctcctcctccgagctggagctggacctggaccgccccaacatcgaggactacctgccctccggctcctccatcaacgagcccgccggcaagctgcgcctgcgcgacctgctggacatctcccccatgctgaccgaggccgccggcgccatcgtggacgactccttcacccgctgcttcaagtccatcccccccgagccctggaactggaacatctacctgttccccctgtggtgcttcggcgtgctgatccgctacctgatcctgttccccgcccgcgtgatcgtgctgaccgtgggctggatcaccgtgatctcctccttcatcaccgtgcgcttcctgctgaagggccacgactccctgcgcatcaagctggagcgcctgatcgtgcagctgttctgctcctccttcgtggcctcctggaccggcgtggtgaagtaccacggcccccgcccctccatccgcccccagcaggtgtacgtggccaaccacacctccatgatcgacttcatcatcctgaaccagatgaccgtgttctccgccatcatgcagaagcaccccggctgggtgggcctgatccagtccaccatcctggagtccgtgggctgcatctggttcaaccgcgccgaggccaaggaccgcgagatcgtggccaagaagctgctggaccacgtgcacggcgagggcaacaaccccctgctgatcttccccgagggcacctgcgtgaacaaccactactccgtgatgttcaagaagggcgccttcgagctgggctgcaccgtgtgccccgtggccatcaagtacaacaagatcttcgtggacgccttctggaactccaagaagcagtccttcaccatgcacctgctgcagctgatgacctcctgggccgtggtgtgcgacgtgtggtacttggagccccagaccctgaagcccggcgagacccccatcgagttcgccgagcgcgtgcgcgacatcatctccgtgcgcgccggcctgaagaaggtgccctgggacggctacctgaagtactcccgcccctcccccaagcaccgcgagcgcaagcagcagtccttcgccgagtccgtgctgcgccgcctggagaagcgctgactcgag

SEQ ID NO: 115 - DNA, 1170 bp, Artificial Sequence
  Description of Artificial Sequence Synthetic polynucleotide
ggtaccatggccaccgccggccgcctgaagccctcctcctccgagctggagctggacctggaccgccccaacatcgaggactacctgccctccggctcctccatcaacgagcccgccggcaagctgcgcctgcgcgacctgctggacatctcccccatgctgaccgaggccgccggcgccatcgtggacgactccttcacccgctgcttcaagtccatcccccccgagccctggaactggaacatctacctgttccccctgtggtgcttcggcgtgctgatccgctacctgatcctgttccccgcccgcgtgatcgtgctgaccgtgggctggatcaccgtgatctcctccttcatcaccgtgcgcttcctgctgaagggccacgactccctgcgcatcaagctggagcgcctgatcgtgcagctgttctgctcctccttcgtggcctcctggaccggcgtggtgaagtaccacggcccccgcccctccatccgcccccagcaggtgtacgtggccaaccacacctccatgatcgacttcatcatcctgaaccagatgaccgtgttctccgccatcatgcagaagcaccccggctgggtgggcctgatccagtccaccatcctggagtccgtgggctgcatctggttcaaccgcgccgaggccaaggaccgcgagatcgtggccaagaagctgctggaccacgtgcacggcgagggcaacaaccccctgctgatcttccccgagggcacctgcgtgaacaaccactactccgtgatgttcaagaagggcgccttcgagctgggctgcaccgtgtgccccgtggccatcaagtacaacaagatcttcgtggacgccttctggaactccaagaagctgtccttcaccatgcacctgctgcagctgatgacctcctgggccgtggtgtgcgacgtgtggtacttggagccccagaccctgaagcccggcgagacccccatcgagttcgccgagcgcgtgcgcgacatcatctccgtgcgcgccggcctgaagaaggtgccctgggacggctacctgaagtactcccgcccctcccccaagcaccgcgagcgcaagcagcagaccttcgccgagtccgtgctgcgccgcctggaggagaagggcaacgtggtgcccaccgtgaactgactcgag

SEQ ID NO: 116 - DNA, 1587 bp, Artificial Sequence
  Description of Artificial Sequence Synthetic polynucleotide
ggtaccatggccatcgccgacggcggcatcatcggcgccgccggctccatctccgccctgaccgccgacaccgaccccccctccctgcgccgccgcaacgtgcccgccggccaggcctccgccgtgtccgccttctccaccgagtccatggccaagcacctgtgcgacccctcccgcgagccctccccctcccccaagtcctccgacgacggcaaggaccccgacatcggctccgtggactccctgaacgagaagccctcctcccccgccgccggcaagggccgcctgcagcacgacctgcgcttcacctaccgcgcctcctcccccgcccaccgcaaggtgaaggagtcccccctgtcctcctccaacatcttcaagcagtcccacgccggcctgttcaacctgtgcgtggtggtgctggtggccgtgaactcccgcctgatcatcgagaacctgatgaagtacggcctgctgatcaagaccggcttctggttctcctcccgctccctgcgcgactggcccctgttcatgtgctgcctgtccctgcccatcttccccctggccgccttcctggtggagaagctggcccagaagaaccgcctgcaggagcccaccgtggtgtgctgccacgtgctgatcacctccgtgtccatcctgtaccccgtgctggtgatcctgcgctgcgactccgccgtgctgtccggcgtggccctgatgctgttcgcctgcatcgtgtggctgaagctggtgtcctacgcccactccaactacgacatgcgctacgtggccaagtccctggacaagggcgagcccgtggtggactccgtgatcgccgaccacccctaccgcgtggactacaaggacctggtgtacttcatggtggcccccaccctgtgctaccagctgtcctaccccctgaccccctgcgtgcgcaagtcctggatcgcccgccaggtgatgaagctggtgctgttcaccggcgtgatgggcttcatcgtggagcagtacatcaaccccatcgtgcagaactccaagcaccccctgaagggcgacctgctgtacgccatcgagcgcgtgctgaagctgtccgtgcccaacctgtacgtgtggctgtgcatgttctactgcttcttccacctgtggctgaacatcctggccgagctgatctgcttcggcgaccgcgagttctacaaggactggtggaacgccaagaccgtggaggagtactggcgcatgtggaacatgcccgtgcacaagtggatggtgcgccacatctacttcccctgcctgcgcaacggcatcccccgcggcgtggccgtgctgatcgccttcctggtgtccgccgtgttccacgagctgtgcatcgccgtgccctgccacgtgttcaagctgtgggccttcatcggcatcatgttccaggtgcccctggtgctggtgtccaactgcctgcagaagaagttccagtcctccatggccggcaacatgttcttctggttcatcttctgcatcttcggccagcccatgtgcgtgctgctgtactaccacgacctgatgaaccgcaagggctcccgcatcgactgactcgag

SEQ ID NO: 117 - DNA, 1599 bp, Artificial Sequence
  Description of Artificial Sequence Synthetic polynucleotide
ggtaccatggccatcgccgacggcggctccgccggcgccgccggctccatctccggctccgacccctccccctccaccgccccctccctgcgccgccgcaacgcctccgccggccaggccttctccaccgagtccatggcccgcgacctgtgcgacccctcccgcgagccctccctgtcccccaagtcctccgacgacggcaaggaccccgccgacgacatcggcgccgccgactccgtggactccggcggcgtgaaggacgagaagccctcctcccaggccgccgccaaggcccgcctggagcacgacctgcgcttcacctaccgcgcctcctcccccgcccaccgcaaggtgaaggagtcccccctgtcctcctccaacatcttcaagcagtcccacgccggcctgttcaacctgtgcgtggtggtgctggtggccgtgaactcccgcctgatcatcgagaacctgatgaagtacggcctgctgatcaagaccggcttctggttctcctcccgctccctgcgcgactggcccctgttcatgtgctgcctgtccctgcccatcttccccctggccgccttcctggtggagaagctggcccagaagaaccgcctgcaggagcccaccgtggtgtgctgccacgtgatcatcacctccgtgtccatcctgtaccccgtgctggtgatcctgcgctgcgactccgccgtgctgtccggcgtggccctgatgctgttcgcctgcatcgtgtggctgaagctggtgtcctacgcccacgccaactacgacatgcgctccgtggccaagtccctggacaagggcgagaccgtggccgactccgtgatcgtggaccacccctaccgcgtggactacaaggacctggtgtacttcatggtggcccccaccctgtgctaccagctgtcctaccccctgaccccctacgtgcgcaagtcctgggtggcccgccaggtgatgaagctggtgctgttcaccggcgtgatgggcttcatcgtggagcagtacatcaaccccatcgtgcagaactccaagcaccccctgaagggcgacctgctgtacgccatcgagcgcgtgctgaagctgtccgtgcccaacctgtacgtgtggctgtgcatgttctactgcttcttccacctgtggctgaacatcctggccgagctgacctgcttcggcgaccgcgagttctacaaggactggtggaacgccaagaccgtggaggagtactggcgcatgtggaacatgcccgtgcacaagtggatggtgcgccacatctacttcccctgcctgcgcaacggcatcccccgcggcgtggccgtgctgatcgccttcctggtgtccgccgtgttccacgagctgtgcatcgccgtgccctgccacgtgttcaagctgtgggccttcatcggcatcatgttccaggtgcccctggtgctggtgtccaactgcctgcagaagaagttccagtcctccatggccggcaacatgttcttctggttcatcttctgcatcttcggccagcccatgtgcgtgctgctgtactaccacgacctgatgaaccgcaagggctcccgcatcgactgactcgag

SEQ ID NO: 118 - DNA, 1404 bp, Artificial Sequence
  Description of Artificial Sequence Synthetic polynucleotide
ggtaccatgggcctggtgtccgtggccgccgccatcggcgtgtccgtgcccgtggcccgcttcctgctgtgcttcctggccaccatccccgtgtccttcctgtggcgcctggtgcccggccgcctgcccaagcacctgtactccgccgcctccggcgccatcctgtcctacctgtccttcggcgcctcctccaacctgcacttcatcgtgcccatgaccctgggctacctgtccatgctgttcttccgccccttctccggcctgctgaccttcttcctgggcttcggctacctgatcggctgccacgtgtactacatgtccggcgacgcctggaaggagggcggcatcgacgccaccggcgccctgatggtgctgaccctgaaggtgatctcctgctccatgaactacaacgacggcctgctgaaggaggagggcctgcgcgagtcccagaagaagaaccgcctgaccaagatgccctccctgatcgagtacttcggctactgcctgtgctgcggctcccacttcgccggccccgtgtacgagatgaaggactacctggagtggaccgagggcaagggcatctggtcccgctcccagaaggagcccaagccctcccccttcggcggcgccctgcgcgccatcatccaggccgccgtgtgcatggccatgtacctgtacctggtgccccaccaccccctgacccgcttcaccgagcccgtgtactacgagtggggcttcttccgccgcctgtcctaccagtacatggccgccctgaccgcccgctggaagtactacttcatctggtccatctccgaggcctccctgatcatctccggcctgggcttctccggctggaccgagtcctccccccccaagccccgctgggaccgcgccaagaacgtggacatcatcggcgtggagttcgccaagtcctccgtgcagctgcccctggtgtggaacatccaggtgtccatctggctgcgccactacgtgtacgaccgcctggtgcagaacggcaagcgccccggcttcttccagctgctggccacccagaccgtgtccgccgtgtggcacggcctgtaccccggctacatcatcttcttcgtgcagtccgccctgatgatcgccggctcccgcgtgatctaccgctggcagcaggccgtgccccccaagatgggcctggtgaagaacatcttcgtgttcttcaacttcgcctacaccctgctggtgctgaactactccgccgtgggcttcatggtgctgtccatgcacgagaccctggcctcctacggctccgtgtactacatcggcaccatcctgcccatcaccctgatcctgctgtcctacgtgatcaagcccggcaagcccgcccgctccaaggcccacaaggagcagtgactcgag

SEQ ID NO: 119 - DNA, 1404 bp, Artificial Sequence
  Description of Artificial Sequence Synthetic polynucleotide
ggtaccatggagctgggctccgtggccgccgccatcggcgtgtccgtgcccgtggcccgcttcctgctgtgcttcctggccaccatccccgtgtccttcctgtggcgcctggtgcccggccgcctgcccaagcacctgtactccgccgcctccggcgccatcctgtcctacctgtccttcggcccctcctccaacctgcacttcatcgtgcccatgaccctgggctacctgtccatgctgttcttccgccccttctccggcctgctgaccttcttcctgggcttcggctacctgatcggctgccacgtgtactacatgtccggcgacgcctggaaggagggcggcatcgacgccaccggcgccctgatggtgctgaccctgaaggtgatctcctgctccatcaactacaacgacggcctgctgaaggaggagggcctgcgcgagtcccagaagaagaaccgcctgaccaagatgccctccctgatcgagtacatcggctactgcctgtgctgcggctcccacttcgccggccccgtgtacgagatgaaggactacctggagtggaccgagggcaagggcgtgtggtcccactccgagaaggagcccaagccctcccccttcggcggcgccctgcgcgccatcatccaggccgccgtgtgcatggccatgtacatgtacctggtgccccaccaccccctgtcccgcttcaccgagcccgtgtactacgagtggggcttcttccgccgcctgtcctaccagtacatggccggcctgaccgcccgctggaagtactacttcatctggtccatctccgaggcctccctgatcatctccggcctgggcttctccggctggaccgagtcctccccccccaagccccgctgggaccgcgccaagaacgtggacatcatcggcgtggagttcgccaagtcctccgtgcagctgcccctggtgtggaacatccaggtgtccacctggctgcgccactacgtgtacgaccgcctggtgcagaacggcaagcgccccggcttcttccagctgctggccacccagaccgtgtccgccatctggcacggcctgtaccccggctacatcatcttcttcgtgcagtccgccctgatgatcgccggctcccgcgtgatctaccgctggcagcaggccgtgccccccaagatgggcctggtgaagaacatcttcgtgttcttcaacttcgcctacaccctgctggtgctgaactactccgccgtgggcttcatggtgctgtccatgcacgagaccctggcctcctacggctccgtgtactacatcggcaccatcctgcccatcaccctgatcctgctgtcctacgtgatcaagcccggcaagcccgcccgctccaaggcccacaaggagcagtgactcgag

SEQ ID NO: 120 - DNA, 1410 bp, Artificial Sequence
  Description of Artificial Sequence Synthetic polynucleotide
ggtaccatggagctggagatcggctccgtggccgccgccatcggcgtgtccgtgcccgtggcccgcttcctgctgtgcttcctggccaccatccccgtgtccttcctgtgccgcctgctgcccgcccgcctgcccaagcacctgtactccgccgcctccggcgccatcctgtcctacctgtccttcggcccctcctccaacctgcacttcatcgtgcccatgtccctgggctacctgtccatgctgttcttccgccccttctccggcctgctgaccttcttcctgggcttcggctacctgatcggctgccacgtgtactacatgtccggcgacgcctggaaggagggcggcatcgacgccaccggcgccctgatggtgctgaccctgaaggtgatctcctgctccatcaactacaacgacggcctgctgaaggaggagggcctgcgcgagtcccagaagaagaaccgcctgaccaagatgccctccctgatcgagtacttcggctactgcctgtgctgcggctcccacttcgccggccccgtgtacgagatgaaggactacctggagtggaccgagggcaagggcatctggtcccgctccgagaaggaccccaagccctcccccttcggcggcgccctgcgcgccatcatccaggccgccgtgtgcatggccatgcacatgtacctggtgccccaccaccccctgacccgcttcaccgagcccgtgtactacgagtggggcttcttccgccgcctgtcctaccagtacatggccgcccagaccgcccgctggaagtactacttcatctggtccatctccgaggcctccctgatcatctccggcctgggcttctccggctggaccgagtcctccccccccaagccccgctgggacaaggccaagaacgtggacatcatcggcgtggagttcgccaagtcctccgtgcagctgcccctggtgtggaacatccaggtgtccacctggctgcgccactacgtgtacgaccgcctggtgcagaacggcaagcgccccggcttcttccagctgctggccacccagaccgtgtccgccgtgtggcacggcctgtaccccggctacatcatcttcttcgtgcagtccgccctgatgatcgccggctcccgcgtgatctaccgctggcagcaggccgtgccccagaagatgggcctggtgaagaacatcttcgtgttcttcaacttcgcctacaccctgctggtgctgaactactccgccgtgggcttcatggtgctgtccatgcacgagaccctggcctcctacggctccgtgtactacatcggcaccatcctgcccatcaccctgatcctgctgtcctacgtgatcaagcccggcaagcccacccgctccaaggtgcacaaggagcagtgactcgag

SEQ ID NO: 121 - DNA, 1410 bp, Artificial Sequence
  Description of Artificial Sequence Synthetic polynucleotide
ggtaccatggagctggagatggagcccctggccgccgccatcggcgtgtccgtggccgtgttccgcttcctggtgtgcttcatcgccaccatccccgtgtccttcatctgccgcctggtgcccggcggcctgccccgccacctgttctccgccgcctccggcgccgtgctgtcctacctgtccttcggcttctcctccaacctgcacttcctggtgcccatgaccctgggctacctgtccatgatcctgttccgccgcttctgcggcatcctgaccttcttcctgggcttcggctacctgatcggctgccacgtgtactacatgtccggcgacgcctggaaggagggcggcatcgacgccaccggcgccctgatggtgctgaccctgaaggtgatctcctgctccatcaactacaacgacggcctgctgaaggaggagggcctgcgcgagtcccagaagaagaaccgcctgatccgcctgccctccctgatcgagtacttcggctactgcctgtgctgcggctcccacttcgccggccccgtgtacgagatgaaggactacctggactggaccgagggcaagggcatctggtcccactccgagaagggccccaagccctcccccctgcgcgccgccctgcgcgccatcatccaggccggcttctgcatggccatgtacctgtacctggtgccccactaccccctgacccgcttcaccgaccccgtgtactacgagtggggcatcctgcgccgcctgtcctaccagtacatggcctccttcaccgcccgctggaagtactacttcatctggtccatctccgaggcctccctgatcatctccggcctgggcttctccggctggaccgagtcctccccccccaagccccgctgggaccgcgccaagaacgtggacatcctgggcgtggagctggccaagtcctccgtgcagatccccctggtgtggaacatccaggtgtccacctggctgcgccactacgtgtacgaccgcctggtgcagaacggcaagcgccccggcttcctgcagctgctggccacccagaccgtgtccgccatctggcacggcgtgtaccccggctacctgatcttcttcgtgcagtccgccctgatgatcgccggctcccgcgccatctaccgctggcagcaggccgtgccccccaagatgtccctggtgaagaacaccctggtgttcttcaacttcgcctacaccctgctggtgctgaactactccgccgtgggcttcatggtgctgtccatgcacgagaccctggcctcctacggctccgtgtactacgtgggcaccatcctgcccgtgaccctgatcctgctgggctacgtgatcaagcccggcaagtccccccgctccaaggcctccaaggagcagtgactcgag

SEQ ID NO: 122 - DNA, 750 bp, Artificial Sequence
  Description of Artificial Sequence Synthetic polynucleotide
ggtaccatgaacttcgacttcctgtccaacatcccctggttcggcgccaaggcctccgacaacgccggctcctccttcggctccgccaccatcgtgatccagcagcccccccccgtgtcccgcggcttcgacatccgccactggggctggccctggtccgtgctgtccgtgctgccctggggcaagcccggctgcgacgagctgcgcgccccccccaccaccatcaaccgccgcctgaagcgcaacgccacctccatgcactcctccgccgtgcgcggcaacgccgaggccgcccgcgtgcgcttccgcccctacgtgtccaaggtgccctggcacaccggcttccgcggcctgctgtcccagctgttcccccgctacggccactactgcggccccaactggtcctccggcaagaacggcggctcccccgtgtgggaccagcgccccatcgactggctggactactgctgctactgccacgacatcggctacgacacccacgaccaggccaagctgctggaggccgacctggccttcctggagtgcctggagcgcccctcctaccccaccaagggcgacgcccacgtggcccacatgtacaagaccatgtgcgtgaccggcctgcgcaacgtgctgatcccctaccgcacccagctgctgcgcctgaactcccgccagcccctgatcgacttcggctggctgtccaacgccgcctggaagggctggaacgcccagaagtcctgactcgag

SEQ ID NO: 123 - DNA, 723 bp, Artificial Sequence
  Description of Artificial Sequence Synthetic polynucleotide
ggtaccatgaacctggacttcctgtccaagatcccctggttcgaggccaaggcctccgagaaccccggcctgaacctgggctccaccaccatcgtgatcaagcagccccgccagggcttcgacatccgccactggggctggccctggtccgtgctgacctggggcaaccgcgtgaccgacgaggtgcacgccccccccaccaccatcaaccgccgcctgaagcgcaacgccaccggccccgccgtgcagggcgacaccgaggccgcccgcctgcgcttccgcccctacgtgtccaaggtgccctggcacaccggcttccgcggcctgctgtcccagctgttcccccgctacggccactactgcggccccaactggtcctccggcaagaacggcggctcccccgtgtgggaccagcgccccatcgactggctggactactgctgctactgccacgacatcggctacgacacccacgaccaggccaagctgctggaggccgacctggccttcctggagtgcctggagcgcccctcctaccccaccaccggcgacgcccacgtggcccacatgtacaagaccatgtgcgtgaccggcctgcgcaacgtgctgatcccctaccgcacccagctgctgcgcctgaacttccgccagcccctgatcgacttcggctggctgtccaacgccgcctggaagggctggtccgcccagaagacctgactcgag

SEQ ID NO: 124 - DNA, 489 bp, Artificial Sequence
  Description of Artificial Sequence Synthetic polynucleotide
ggtaccatggtgcacctgccccacaccctgaagctgggcctggtgatcgccatctccatctccggcctgtgcttctcctccacccccgcccgcgccctgaacgtgggcatccaggccgccggcgtgaccgtgtccgtgggcaagggctgctcccgcaagtgcgagtccgacttctgcaaggtgccccccttcctgcgctacggcaagtactgcggcctgatgtactccggctgccccggcgagaagccctgcgacggcctggacgcctgctgcatgaagcacgacgcctgcgtgcaggccaagaacaacgactacctgtcccaggagtgctcccagaacctgctgaactgcatggcctccttccgcatgtccggcggcaagcagttcaagggctccacctgccaggtggacgaggtggtggacgtgctgaccgtggtgatggaggccgccctgctggccggccgctacctgcacaagccctgactcgag

SEQ ID NO: 125 - DNA, 489 bp, Artificial Sequence
  Description of Artificial Sequence Synthetic polynucleotide
ggtaccatggtgcacctgccccacaccctgaagctgggcctggtgatcgccatctccatctccggcctgtgcctgtcctccacccccgcccgcgccctgaacgtgggcatccaggccgccggcgtgaccgtgtccgtgggcaagggctgctcccgcaagtgcgagtccgacttctgcaaggtgccccccttcctgcgctacggcaagtactgcggcctgatgtactccggctgccccggcgagaagccctgcgacggcctggacgcctgctgcatgaagcacgacgcctgcgtgcaggccaagaacgacgactacctgtcccaggagtgctcccagaacctgctgaactgcatggcctccttccgcatgtccggcggcaagcagttcaagggctccacctgccaggtggacgaggtggtggacgtgctgaccgtggtgatggaggccgccctgctggccggccgctacctgcacaagccctgactcgag

SEQ ID NO: 126 - DNA, 7557 bp, Artificial Sequence
  Description of Artificial Sequence Synthetic polynucleotide
gtttaaacgccggtcaccacccgcatgctcgtactacagcgcacgcaccgcttcgtgatccaccgggtgaacgtagtcctcgacggaaacatctggttcgggcctcctgcttgcactcccgcccatgccgacaacctttctgctgttaccacgacccacaatgcaacgcgacacgaccgtgtgggactgatcggttcactgcacctgcatgcaattgtcacaagcgcttactccaattgtattcgtttgttttctgggagcagttgctcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaactagtatgcagaccgcccaccagcgcccccccaccgagggccactgcttcggcgcccgcctgcccaccgcctcccgccgcgccgtgcgccgcgcctggtcccgcatcgcccgcgggcgcgccgccgccgccgccgacgccaaccccgcccgccccgagcgccgcgtggtgatcaccggccagggcgtggtgacctccctgggccagaccatcgagcagttctactcctccctgctggagggcgtgtccggcatctcccagatccagaagttcgacaccaccggctacaccaccaccatcgccggcgagatcaagtccctgcagctggacccctacgtgcccaagcgctgggccaagcgcgtggacgacgtgatcaagtacgtgtacatcgccggcaagcaggccctggagtccgccggcctgcccatcgaggccgccggcctggccggcgccggcctggaccccgccctgtgcggcgtgctgatcggcaccgccatggccggcatgacctccttcgccgccggcgtggaggccctgacccgcggcggcgtgcgcaagatgaaccccttctgcatccccttctccatctccaacatgggcggcgccatgctggccatggacatcggcttcatgggccccaactactccatctccaccgcctgcgccaccggcaactactgcatcctgggcgccgccgaccacatccgccgcggcgacgccaacgtgatgctggccggcggcgccgacgccgccatcatcccctccggcatcggcggcttcatcgcctgcaaggccctgtccaagcgcaacgacgagcccgagcgcgcctcccgcccctgggacgccgaccgcgacggcttcgtgatgggcgagggcgccggcgtgctggtgctggaggagctggagcacgccaagcgccgcggcgccaccatcctggccgagctggtgggcggcgccgccacctccgacgcccaccacatgaccgagcccgacccccagggccgcggcgtgcgcctgtgcctggagcgcgccctggagcgcgcccgcctggcccccgagcgcgtgggctacgtgaacgcccacggcacctccacccccgccggcgacgtggccgagtaccgcgccatccgcgccgtgatcccccaggactccctgcgcatcaactccaccaagtccatgatcggccacctgctgggcggcgccggcgccgtggaggccgtggccgccatccaggccctgcgcaccggctggctgcaccccaacctgaacctggagaaccccgcccccggcgtggaccccgtggtgctggtgggcccccgcaaggagcgcgccgaggacctggacgtggtgctgtccaactccttcggcttcggcggccacaactcctgcgtgatcttccgcaagtacgacgagatggactacaaggaccacgacggcgactacaaggaccacgacatcgactacaaggacgacgacgacaagtgaatcgatgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggagagctccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctaggtgatatcgaattcctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacactagtatggctatcaagacgaacaggcagcctgtggagaagcctccgttcacgatcgggacgctgcgcaaggccatccccgcgcactgtttcgagcgctcggcgcttcgtagcagcatgtacctggcctttgacatcgcggtcatgtccctgctctacgtcgcgtcgacgtacatcgaccctgcaccggtgcctacgtgggtcaagtacggcatcatgtggccgctctactggttcttccaggtgtgtttgagggttttggttgcccgtattgaggtcctggtggcgcgcatggaggagaaggcgcctgtcccgctgacccccccggctaccctcccggcaccttccagggcgcgtacgggaagaaccagtagagcggccacatgatgccgtacttgacccacgtaggcaccggtgcagggtcgatgtacgtcgacgcgacgtagagcagggacatgaccgcgatgtcaaaggccaggtacatgctgctacgaagcgccgagcgctcgaaacagtgcgcggggatggccttgcgcagcgtcccgatcgtgaacggaggcttctccacaggctgcctgttcgtcttgatagccatctcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagagctcgatctaagtaagattcgaagcgctcgaccgtgccggacggactgcagccccatgtcgtagtgaccgccaatgtaagtgggctggcgtttccctgtacgtgagtcaacgtcactgcacgcgcaccaccctctcgaccggcaggaccaggcatcgcgagatacagcgcgagccagacacggagtgccgagctatgcgcacgctccaactaggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactcgtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcaggatccttagggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtcctgggcgaagaacgagggaatttgtgggtaaaacaagcatcgtctctcaggccccggcgcagtggccgttaaagtccaagaccgtgaccaggcagcgcagcgcgtccgtgtgcgggccctgcctggcggctcggcgtgccaggctcgagagcagctccctcaggtcgccttggacggcctctgcgaggccggtgagggcctgcaggagcgcctcgagcgtggcagtggcggtcgtatccgggtcgccggtcaccgcctgcgactcgccatccgaagagcgtttaaac

SEQ ID NO: 127 - DNA, 8094 bp, Artificial Sequence
  Description of Artificial Sequence Synthetic polynucleotide
gaagagcgcccaatgtttaaacctcttttgctgcgtctcctcaggcttgggggcctccttgggcttgggtgccgccatgatctgcgcgcatcagagaaacgttgctggtaaaaaggagcgcccggctgcgcaatatatatataggcatgccaacacagcccaacctcactcgggagcccgtcccaccacccccaagtcgcgtgccttgacggcatactgctgcagaagcttcatgagaatgatgccgaacaagaggggcacgaggacccaatcccggacatccttgtcgataatgatctcgtgagtccccatcgtccgcccgacgctccggggagcccgccgatgctcaagacgagagggccctcgaccaggaggggctggcccgggcgggcactggcgtcgaaggtgcgcccgtcgttcgcctgcagtcctatgccacaaaacaagtcttctgacggggtgcgtttgctcccgtgcgggcaggcaacagaggtattcaccctggtcatggggagatcggcgatcgagctgggataagagatacggtcccgcgcaaggatcgctcatcctggtctgagccggacagtcattctggcaagcaatgacaacttgtcaggaccggaccgtgccatatatttctcacctagcgccgcaaaacctaacaatttgggagtcactgtgccactgagttcgactggtagctgaatggagtcgctgctccactaaacgaattgtcagcaccgccagccggccgaggacccgagtcatagcgagggtagtagcgcgccactagtatggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccgccggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtgaatcgatggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtgagctccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctaggtacgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcatcaaaccgagttgtgcacaagggcgactaattttttagtcgggaaacaacccgcttccagaagcatccggacgggggtagcgaggctgtgtcgagcgccgtggggatctggccggtgaggtgcccgaaatccgtgtacagctcagcggctgggatcatcgacccccgggatcatcgaccccgtgggccgggcccccggaccctataactaaaagccgacgccagtgcaaaaccacaaacatttactccttaatcctccctcctccttcatacacacccacaagtaatcaactcacccatatggccatcgccgccgccgccgtgatcgtgcccctgggcctgctgttcttcatctccggcctggtggtgaacctgatccaggccctgtgcttcgtgctgatccgccccctgtccaagaacacctaccgcaagatcaaccgcgtggtggccgagctgctgtggctggagctgatctggctggtggactggtgggccggcgtgaagatcaaggtgttcatggaccccgagtccttcaacctgatgggcaaggagcacgccctggtggtggccaaccaccgctccgacatcgactggctggtgggctggctgctggcccagcgctccggctgcctgggctccgccctggccgtgatgaagaagtcctccaagttcctgcccgtgatcggctggtccatgtggttctccgagtacctgttcctggagcgctcctgggccaaggacgagaacaccctgaaggccggcctgcagcgcctgaaggacttcccccgccccttctggctggccttcttcgtggagggcacccgcttcacccaggccaagttcctggccgcccaggagtacgccgcctcccagggcctgcccatcccccgcaacgtgctgatcccccgcaccaagggcttcgtgtccgccgtgtcccacatgcgctccttcgtgcccgccatctacgacatgaccgtggccatccccaagtcctccccctcccccaccatgctgcgcctgttcaagggccagccctccgtggtgcacgtgcacatcaagcgctgcctgatgaaggagctgcccgagaccgacgaggccgtggcccagtggtgcaaggacatgttcgtggagaaggacaagctgctggacaagcacatcgccgaggacaccttctccgaccagcccatgcaggacctgggccgccccatcaagtccctgctggtggtggcctcctgggcctgcctgatggcctacggcgccctgaagttcctgcagtgctcctccctgctgtcctcctggaagggcatcgccttcttcctggtgggcctggccatcgtgaccatcctgatgcacatcctgatcctgttctcccagtccgagcgctccacccccgccaaggtggcccccggcaagcccaagaacgacggcgagacctccgaggcccgccgcgacaagcagcagtgaatgcatatgtggagatgtagggtggtcgactcgttggaggtgggtgtttttttttatcgagtgcgcggcgcggcaaacgggtccctttttatcgaggtgttcccaacgccgcaccgccctcttaaaacaacccccaccaccacttgtcgaccttctcgtttgttatccgccacggcgccccggaggggcgtcgtctggccgcgcgggcagctgtatcgccgcgctcgctccaatggtgtgtaatcttggaaagataataatcgatggatgaggaggagagcgtgggagatcagagcaaggaatatacagttggcacgaagcagcagcgtactaagctgtagcgtgttaagaaagaaaaactcgctgttaggctgtattaatcaaggagcgtatcaataattaccgaccctatacctttatctccaacccaatcgcggcttaaggatctaagtaagattcgaagcgctcgaccgtgccggacggactgcagccccatgtcgtagtgaccgccaatgtaagtgggctggcgtttccctgtacgtgagtcaacgtcactgcacgcgcaccaccctctcgaccggcaggaccaggcatcgcgagatacagcgcgagccagacacggagtgccgagctatgcgcacgctccaactaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattgacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggatcctgaggacagggtggttggctggatggggaaacgctggtcgcgggattcgatcctgctgcttatatcctccctggaagcacacccacgactctgaagaagaaaacgtgcacacacacaacccaaccggccgaatatttgcttccttatcccgggtccaagagagactgcgatgcccccctcaatcagcatcctcctccctgccgcttcaatcttccctgcttgcctgcgcccgcggtgcgccgtctgcccgcccagtcagtcactcctgcacaggccccttgtgcgcagtgctcctgtaccctttaccgctccttccattctgcgaggccccctattgaatgtattcgttgcctgtgtggccaagcgggctgctgggcgcgccgccgtcgggcagtgctcggcgactttggcggaagccgattgttcttctgtaagccacgcgcttgctgctttgggaagagaagggggggggtactgaatggatgaggaggagaaggaggggtattggtattatctgagttggggaggcagggagagttggaaaatgtaagtggcacgacgggcaaggagaatggtgagcatgtgcatggtgatgtcgttggtcgaggacgatcctgcacgcgtgtatctgatgtagaatacggcaatcaccctagtctacatctataccttctccgtataacgccctttccaaatgccctcccgtttctctcctattcttgatccacatgatgaccctggcactatttcaagggctggagaagagcgtttaaac

SEQ ID NO: 128 - DNA, 10062 bp, Artificial Sequence
  Description of Artificial Sequence Synthetic polynucleotide
gctcttcgcgaaggtcattttccagaacaacgaccatggcttgtcttagcgatcgctcgaatgactgctagtgagtcgtacgctcgacccagtcgctcgcaggagaacgcggcaactgccgagcttcggcttgccagtcgtgactcgtatgtgatcaggaatcattggcattggtagcattataattcggcttccgcgctgtttatgggcatggcaatgtctcatgcagtcgaccttagtcaaccaattctgggtggccagctccgggcgaccgggctccgtgtcgccgggcaccacctcctgccatgagtaacagggccgccctctcctcccgacgttggccaactgaataccgtgtcttggggccctacatgatgggctgcctagtcgggcgggacgcgcaactgcccgcgcaatctgggacgtggtctgaatcctccaggcgggtttccccgagaaagaaagggtgccgatttcaaagcagagccatgtgccgggccctgtggcctgtgttggcgcctatgtagtcaccccccctcacccaattgtcgccagtttgcgcaatccataaactcaaaactgcagcttctgagctgcgctgttcaagaacacctctggggtttgctcacccgcgaggtcgacggtacctccctccgtctctgcactctggcgcccctcctccgtctcgtggactgacggacgagagtctgggcgccgcttttctatccacaccgccctttccgcatcgaagacaccacccatcgtgccgccaggtcttccccaatcacccgccctgtggtcctctctcccagccgtgtttggtcgctgcgtccacatttttccattcgtgccccacgatcctcgcccatcttggcgccttggataggcacccttttttcagcacgccctggtgtgtagcacaacctgacctctctctaccgcatcgcctccctcccacacctcagttgactccctcgtcgcacgttgcacccgcaagctccccatttcatcctattgacaatcgcacactgtacatgtatgctcattattttgcaaaaaaacagggggtcggttcactcctggcagacgacgcggtgctgccgcgcgccgctgaggcggcgtcgcgacggcaacacccatcgcaccgcacgtcgacgagtcaacccaccctgctcaacggtgatctccccatcgcgacaccccccgtgaccgtactatgtgcgtccatacgcaacatgaaaaggaccttggtccccggaggcggcgagctcgtaatcccgaggttggccccgcttccgctggacacccatcgcatcttccggctcgcccgctgtcgagcaagcgccctcgtgcgcgcaacccttgtggtgcctgcccgcagagccgggcataaaggcgagcaccacacccgaaccagtccaatttgctttctgcattcactcaccaacttttacatccacacatcgtactaccacacctgcccagtcgggtttgatttctattgcaaaggtgcgggggggttggcgcactgcgtgggttgtgcagccggccgccgcggctgtacccagcgatcaggtagcttgggctgtatcttctcaagcattaccttgtcctgggcgtaggtttgcctctagaatggccgcgtccgtccactgcaccctgatgtccgtggtctgcaacaacaagaaccactccgcccgccccaagctgcccaactcctccctgctgcccggcttcgacgtggtggtccaggccgcggccacccgcttcaagaaggagacgacgaccacccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcctgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgatacgtaacagacgaccttggcaggcgtcgggtagggaggtggtggtgatggcgtctcgatgccatcgcacgcatccaacgaccgtatacgcatcgtccaatgaccgtcggtgtcctctctgcctccgttttgtgagatgtctcaggcttggtgcatcctcgggtggccagccacgttgcgcgtcgtgctgcttgcctctcttgcgcctctgtggtactggaaaatatcatcgaggcccgtttttttgctcccatttcctttccgctacatcttgaaagcaaacgacaaacgaagcagcaagcaaagagcacgaggacggtgaacaagtctgtcacctgtatacatctatttccccgcgggtgcacctactctctctcctgccccggcagagtcagctgccttacgtgacggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctaggctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccatctccgactcccccgagatcctgggctccaccgccaccgtgacctcctcctcccactccgactccgacctgaacctgctgtccatccgccgccgcacctccaccaccgccgccgcccgcgcccccgaccgcgacgactccggcaacggcgaggccgtggacgaccgcgaccgcgtggagtccgccaacctgatgtccaacgtggccgagaacgccaacgagatgcccaactcctccgacacccgcttcacctaccgcccccgcgtgcccgcccaccgccgcatcaaggagtcccccctgtcctccggcgccatcttcaagcagtcccacgccggcctgttcaacctgtgcatcgtggtgctggtggccgtgaactcccgcctgatcatcgagaacctgatgaagtacggctggctgatccgctccggcttctggttctcctcccgctccctgtccgactggcccctgttcatgtgctgcctgaccctgcccatcttccccctggccgccttcgtggtggagaagctggtgcagcgcaactacatctccgagcccgtggtggtgttcctgcacgccatcatctccaccaccgccgtgctgtaccccgtgatcgtgaacctgcgctgcgactccgccttcctgtccggcgtggccctgatgctgttcgcctgcatcgtgtggctgaagctggtgtcctacgcccacaccaacaacgacatgcgcgccctggccaagtccgccgagaagggcgacgtggacccctcctacgacgtgtccttcaagtccctggcctacttcatggtggcccccaccctgtgctaccagcagtcctacccccgcacccccgccgtgcgcaagtcctgggtggtgcgccagttcatcaagctgatcgtgttcaccggcctgatgggcttcatcatcgagcagtacatcaaccccatcgtgcagaactcccagcaccccctgaagggcaacctgctgtacgccatcgagcgcgtgctgaagctgtccgtgcccaacctgtacgtgtggctgtgcatgttctactgcttcttccacctgtggctgaacatcctggccgagctgctgcgcttcggcgaccgcgagttctacaaggactggtggaacgccaagaccgtggaggagtactggcgcatgtggaacatgcccgtgcacaagtggatggtgcgccacatctacttcccctgcctgcgcaacggcatccccaagggcgtggccatcgtgatcgccttcctggtgtccgccgtgttccacgagctgtgcatcgccgtgccctgccacatgttcaagctgtgggccttcatcggcatcatgttccaggtgcccctggtgctgatcaccaactacctgcaggacaagttccgctcctccatggtgggcaacatgatcttctggttcatcttctccatcctgggccagcccatgtgcgtgctgctgtactaccacgacctgatgaaccgcaagggcaaggccgactgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacttaaggatctaagtaagattcgaagcgctcgaccgtgccggacggactgcagccccatgtcgtagtgaccgccaatgtaagtgggctggcgtttccctgtacgtgagtcaacgtcactgcacgcgcaccaccctctcgaccggcaggaccaggcatcgcgagatacagcgcgagccagacacggagtgccgagctatgcgcacgctccaactagatatcatgtggatgatgagcatgaattcgggagcagttgtcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccgccggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtgaatcgatggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtgagctcctcactcagcgcgcctgcgcggggatgcggaacgccgccgccgccttgtcttttgcacgcgcgactccgtcgcttcgcgggtggcacccccattgaaaaaaacctcaattctgtttgtggaagacacggtgtacccccaaccacccacctgcacctctattattggtattattgacgcgggagcgggcgttgtactctacaacgtagcgtctctggttttcagctggctcccaccattgtaaattcttgctaaaatagtgcgtggttatgtgagaggtatggtgtaacagggcgtcagtcatgttggttttcgtgctgatctcgggcacaaggcgtcgtcgacgtgacgtgcccgtgatgagagcaataccgcgctcaaagccgacgcatggcctttactccgcactccaaacgactgtcgctcgtatttttcggatatctattttttaagagcgagcacagcgccgggcatgggcctgaaaggcctcgcggccgtgctcgtggtgggggccgcgagcgcgtggggcatcgcggcagtgcaccaggcgcagacggaggaacgcatggtgagtgcgcatcacaagatgcatgtcttgttgtctgtactataatgctagagcatcaccaggggcttagtcatcgcacctgctttggtcattacagaaattgcacaagggcgtcctccgggatgaggagatgtaccagctcaagctggagcggcttcgagccaagcaggagcgcggcgcatgacgacctacccacatgcgaagagc

SEQ ID NO: 129 - DNA, 9540 bp, Artificial Sequence
  Description of Artificial Sequence Synthetic polynucleotide
gctcttcgcgaaggtcattttccagaacaacgaccatggcttgtcttagcgatcgctcgaatgactgctagtgagtcgtacgctcgacccagtcgctcgcaggagaacgcggcaactgccgagcttcggcttgccagtcgtgactcgtatgtgatcaggaatcattggcattggtagcattataattcggcttccgcgctgtttatgggcatggcaatgtctcatgcagtcgaccttagtcaaccaattctgggtggccagctccgggcgaccgggctccgtgtcgccgggcaccacctcctgccatgagtaacagggccgccctctcctcccgacgttggccaactgaataccgtgtcttggggccctacatgatgggctgcctagtcgggcgggacgcgcaactgcccgcgcaatctgggacgtggtctgaatcctccaggcgggtttccccgagaaagaaagggtgccgatttcaaagcagagccatgtgccgggccctgtggcctgtgttggcgcctatgtagtcaccccccctcacccaattgtcgccagtttgcgcaatccataaactcaaaactgcagcttctgagctgcgctgttcaagaacacctctggggtttgctcacccgcgaggtcgacggtacctccctccgtctctgcactctggcgcccctcctccgtctcgtggactgacggacgagagtctgggcgccgcttttctatccacaccgccctttccgcatcgaagacaccacccatcgtgccgccaggtcttccccaatcacccgccctgtggtcctctctcccagccgtgtttggtcgctgcgtccacatttttccattcgtgccccacgatcctcgcccatcttggcgccttggataggcacccttttttcagcacgccctggtgtgtagcacaacctgacctctctctaccgcatcgcctccctcccacacctcagttgactccctcgtcgcacgttgcacccgcaagctccccatttcatcctattgacaatcgcacactgtacatgtatgctcattattttgcaaaaaaacagggggtcggttcactcctggcagacgacgcggtgctgccgcgcgccgctgaggcggcgtcgcgacggcaacacccatcgcaccgcacgtcgacgagtcaacccaccctgctcaacggtgatctccccatcgcgacaccccccgtgaccgtactatgtgcgtccatacgcaacatgaaaaggaccttggtccccggaggcggcgagctcgtaatcccgaggttggccccgcttccgctggacacccatcgcatcttccggctcgcccgctgtcgagcaagcgccctcgtgcgcgcaacccttgtggtgcctgcccgcagagccgggcataaaggcgagcaccacacccgaaccagtccaatttgctttctgcattcactcaccaacttttacatccacacatcgtactaccacacctgcccagtcgggtttgatttctattgcaaaggtgcgggggggttggcgcactgcgtgggttgtgcagccggccgccgcggctgtacccagcgatcaggtagcttgggctgtatcttctcaagcattaccttgtcctgggcgtaggtttgcctctagaatggccgcgtccgtccactgcaccctgatgtccgtggtctgcaacaacaagaaccactccgcccgccccaagctgcccaactcctccctgctgcccggcttcgacgtggtggtccaggccgcggccacccgcttcaagaaggagacgacgaccacccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcctgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgatacgtaacagacgaccttggcaggcgtcgggtagggaggtggtggtgatggcgtctcgatgccatcgcacgcatccaacgaccgtatacgcatcgtccaatgaccgtcggtgtcctctctgcctccgttttgtgagatgtctcaggcttggtgcatcctcgggtggccagccacgttgcgcgtcgtgctgcttgcctctcttgcgcctctgtggtactggaaaatatcatcgaggcccgtttttttgctcccatttcctttccgctacatcttgaaagcaaacgacaaacgaagcagcaagcaaagagcacgaggacggtgaacaagtctgtcacctgtatacatctatttccccgcgggtgcacctactctctctcctgccccggcagagtcagctgccttacgtgacggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctaggctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatgaccggcgaggagatggaggagcgcaaggccaccggctaccgcgagttctccggccgccacgagttcccctccaacaccatgcacgccctgctggccatgggcatctggctgggcgccatccacttcaacgccctgctgctgctgttctccttcctgttcctgcccttctccaagttcctggtggtgttcggcctgctgctgctgttcatgatcctgcccatcgacccctactccaagttcggccgccgcctgtcccgctacatctccaagcacgcctgctcctacttccccatcaccctgcacgtggaggacatccacgccttccaccccgaccgcgcctacgtgttcggcttcgagccccactccgtgctgcccatcggcgtggtggccctggccgacctgaccggcttcatgcccctgcccaagatcaaggtgctggcctcctccgccgtgttctacacccccttcctgcgccacatctggacctggctgggcctgacccccgccaccaagaagaacttctcctccctgctggacgccggctactcctgcatcctggtgcccggcggcgtgcaggagaccttccacatggagcccggctccgagatcgccttcctgcgcgcccgccgcggcttcgtgcgcatcgccatggagatgggctcccccctggtgcccgtgttctgcttcggccagtcccacgtgtacaagtggtggaagcccggcggcaagttctacctgcagttctcccgcgccatcaagttcacccccatcttcttctggggcatcttcggctcccccctgccctaccagcaccccatgcacgtggtggtgggcaagcccatcgacgtgaagaagaacccccagcccatcgtggaggaggtgatcgaggtgcacgaccgcttcgtggaggccctgcaggacctgttcgagcgccacaaggcccaggtgggcttcgccgacctgcccctgaagatcctgtgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacttaaggatctaagtaagattcgaagcgctcgaccgtgccggacggactgcagccccatgtcgtagtgaccgccaatgtaagtgggctggcgtttccctgtacgtgagtcaacgtcactgcacgcgcaccaccctctcgaccggcaggaccaggcatcgcgagatacagcgcgagccagacacggagtgccgagctatgcgcacgctccaactagatatcatgtggatgatgagcatgaattcgggagcagttgtcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccgccggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtgaatcgatggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtgagctcctcactcagcgcgcctgcgcggggatgcggaacgccgccgccgccttgtcttttgcacgcgcgactccgtcgcttcgcgggtggcacccccattgaaaaaaacctcaattctgtttgtggaagacacggtgtacccccaaccacccacctgcacctctattattggtattattgacgcgggagcgggcgttgtactctacaacgtagcgtctctggttttcagctggctcccaccattgtaaattcttgctaaaatagtgcgtggttatgtgagaggtatggtgtaacagggcgtcagtcatgttggttttcgtgctgatctcgggcacaaggcgtcgtcgacgtgacgtgcccgtgatgagagcaataccgcgctcaaagccgacgcatggcctttactccgcactccaaacgactgtcgctcgtatttttcggatatctattttttaagagcgagcacagcgccgggcatgggcctgaaaggcctcgcggccgtgctcgtggtgggggccgcgagcgcgtggggcatcgcggcagtgcaccaggcgcagacggaggaacgcatggtgagtgcgcatcacaagatgcatgtcttgttgtctgtactataatgctagagcatcaccaggggcttagtcatcgcacctgctttggtcattacagaaattgcacaagggcgtcctccgggatgaggagatgtaccagctcaagctggagcggcttcgagccaagcaggagcgcggcgcatgacgacctacccacatgcgaagagc

SEQ ID NO: 130 - DNA, 7158 bp, Artificial Sequence
  Description of Artificial Sequence Synthetic polynucleotide
gctcttcgcgaaggtcattttccagaacaacgaccatggcttgtcttagcgatcgctcgaatgactgctagtgagtcgtacgctcgacccagtcgctcgcaggagaacgcggcaactgccgagcttcggcttgccagtcgtgactcgtatgtgatcaggaatcattggcattggtagcattataattcggcttccgcgctgtttatgggcatggcaatgtctcatgcagtcgaccttagtcaaccaattctgggtggccagctccgggcgaccgggctccgtgtcgccgggcaccacctcctgccatgagtaacagggccgccctctcctcccgacgttggccaactgaataccgtgtcttggggccctacatgatgggctgcctagtcgggcgggacgcgcaactgcccgcgcaatctgggacgtggtctgaatcctccaggcgggtttccccgagaaagaaagggtgccgatttcaaagcagagccatgtgccgggccctgtggcctgtgttggcgcctatgtagtcaccccccctcacccaattgtcgccagtttgcgcaatccataaactcaaaactgcagcttctgagctgcgctgttcaagaacacctctggggtttgctcacccgcgaggtcgacggtacctccctccgtctctgcactctggcgcccctcctccgtctcgtggactgacggacgagagtctgggcgccgcttttctatccacaccgccctttccgcatcgaagacaccacccatcgtgccgccaggtcttccccaatcacccgccctgtggtcctctctcccagccgtgtttggtcgctgcgtccacatttttccattcgtgccccacgatcctcgcccatcttggcgccttggataggcacccttttttcagcacgccctggtgtgtagcacaacctgacctctctctaccgcatcgcctccctcccacacctcagttgactccctcgtcgcacgttgcacccgcaagctccccatttcatcctattgacaatcgcacactgtacatgtatgctcattattttgcaaaaaaacagggggtcggttcactcctggcagacgacgcggtgctgccgcgcgccgctgaggcggcgtcgcgacggcaacacccatcgcaccgcacgtcgacgagtcaacccaccctgctcaacggtgatctccccatcgcgacaccccccgtgaccgtactatgtgcgtccatacgcaacatgaaaaggaccttggtccccggaggcggcgagctcgtaatcccgaggttggccccgcttccgctggacacccatcgcatcttccggctcgcccgctgtcgagcaagcgccctcgtgcgcgcaacccttgtggtgcctgcccgcagagccgggcataaaggcgagcaccacacccgaaccagtccaatttgctttctgcattcactcaccaacttttacatccacacatcgtactaccacacctgcccagtcgggtttgatttctattgcaaaggtgcgggggggttggcgcactgcgtgggttgtgcagccggccgccgcggctgtacccagcgatcaggtagcttgggctgtatcttctcaagcattaccttgtcctgggcgtaggtttgcctctagaatggccgcgtccgtccactgcaccctgatgtccgtggtctgcaacaacaagaaccactccgcccgccccaagctgcccaactcctccctgctgcccggcttcgacgtggtggtccaggccgcggccacccgcttcaagaaggagacgacgaccacccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcctgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgatacgtaacagacgaccttggcaggcgtcgggtagggaggtggtggtgatggcgtctcgatgccatcgcacgcatccaacgaccgtatacgcatcgtccaatgaccgtcggtgtcctctctgcctccgttttgtgagatgtctcaggcttggtgcatcctcgggtggccagccacgttgcgcgtcgtgctgcttgcctctcttgcgcctctgtggtactggaaaatatcatcgaggcccgtttttttgctcccatttcctttccgctacatcttgaaagcaaacgacaaacgaagcagcaagcaaagagcacgaggacggtgaacaagtctgtcacctgtatacatctatttccccgcgggtgcacctactctctctcctgccccggcagagtcagctgccttacgtgacggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggggagcagttgtcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccgccggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtgaatcgatggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtgagctcctcactcagcgcgcctgcgcggggatgcggaacgccgccgccgccttgtcttttgcacgcgcgactccgtcgcttcgcgggtggcacccccattgaaaaaaacctcaattctgtttgtggaagacacggtgtacccccaaccacccacctgcacctctattattggtattattgacgcgggagcgggcgttgtactctacaacgtagcgtctctggttttcagctggctcccaccattgtaaattcttgctaaaatagtgcgtggttatgtgagaggtatggtgtaacagggcgtcagtcatgttggttttcgtgctgatctcgggcacaaggcgtcgtcgacgtgacgtgcccgtgatgagagcaataccgcgctcaaagccgacgcatggcctttactccgcactccaaacgactgtcgctcgtatttttcggatatctattttttaagagcgagcacagcgccgggcatgggcctgaaaggcctcgcggccgtgctcgtggtgggggccgcgagcgcgtggggcatcgcggcagtgcaccaggcgcagacggaggaacgcatggtgagtgcgcatcacaagatgcatgtcttgttgtctgtactataatgctagagcatcaccaggggcttagtcatcgcacctgctttggtcattacagaaattgcacaagggcgtcctccgggatgaggagatgtaccagctcaagctggagcggcttcgagccaagcaggagcgcggcgcatgacgacctacccacatgcgaagagc

SEQ ID NO: 131 - DNA, 6046 bp, Artificial Sequence
  Description of Artificial Sequence Synthetic polynucleotide
caccggcgcgctgcttcgcgtgccgggtgcagcaatcagatccaagtctgacgacttgcgcgcacgcgccggatccttcaattccaaagtgtcgtccgcgtgcgcttcttcgccttcgtcctcttgaacatccagcgacgcaagcgcagggcgctgggcggctggcgtcccgaaccggcctcggcgcacgcggctgaaattgccgatgtcggcaatgtagtgccgctccgcccacctctcaattaagtttttcagcgcgtggttgggaatgatctgcgctcatggggcgaaagaaggggttcagaggtgctttattgttactcgactgggcgtaccagcattcgtgcatgactgattatacatacaaaagtacagctcgcttcaatgccctgcgattcctactcccgagcgagcactcctctcaccgtcgggttgcttcccacgaccacgccggtaagagggtctgtggcctcgcgcccctcgcgagcgcatctttccagccacgtctgtatgattttgcgctcatacgtctggcccgtcgaccccaaaatgacgggatcctgcataatatcgcccgaaatgggatccaggcattcgtcaggaggcgtcagccccgcgggagatgccggtcccgccgcattggaaaggtgtagagggggtgaatcccccatttcatgaaatgggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactcgtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgcctcccagctgcgcaagcccgccctggaccccctgcgcgccgtgatctccgccgaccagggctccatctcccccgtgaactcctgcacccccgccgaccgcctgcgcgccggccgcctgatggaggacggctactcctacaaggagaagttcatcgtgcgctcctacgaggtgggcatcaacaagaccgccaccgtggagaccatcgccaacctgctgcaggaggtggcctgcaaccacgtgcagaagtgcggcttctccaccgacggcttcgccaccaccctgaccatgcgcaagctgcacctgatctgggtgaccgcccgcatgcacatcgagatctacaagtaccccgcctggtccgacgtggtggagatcgagacctggtgccagtccgagggccgcatcggcacccgccgcgactggatcctgcgcgactccgccaccaacgaggtgatcggccgcgccacctccaagtgggtgatgatgaaccaggacacccgccgcctgcagcgcgtgaccgacgaggtgcgcgacgagtacctggtgttctgcccccgcgagccccgcctggccttccccgaggagaacaactcctccctgaagaagatccccaagctggaggaccccgcccagtactccatgctggagctgaagccccgccgcgccgacctggacatgaaccagcacgtgaacaacgtgacctacatcggctgggtgctggagtccatcccccaggagatcatcgacacccacgagctgcaggtgatcaccctggactaccgccgcgagtgccagcaggacgacatcgtggactccctgaccacctccgagatccccgacgaccccatctccaagttcaccggcaccaacggctccgccatgtcctccatccagggccacaacgagtcccagttcctgcacatgctgcgcctgtccgagaacggccaggagatcaaccgcggccgcacccagtggcgcaagaagtcctcccgcatggactacaaggaccacgacggcgactacaaggaccacgacatcgactacaaggacgacgacgacaagtgaatcgatggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtgagctccgcgcctgcgcgaggacgcagaacaacgctgccgccgtgtcttttgcacgcgcgactccggcgcttcgctggtggcacccccataaagaaaccctcaattctgtttgtggaagacacggtgtacccccacccacccacctgcacctctattattggtattattgacgcgggagtgggcgttgtaccctacaacgtagcttctctagttttcagctggctcccaccattgtaaattcatgctagaatagtgcgtggttatgtgagaggtatagtgtgtctgagcagacggggcgggatgcatgtcgtggtggtgatctttggctcaaggcgtcgtcgacgtgacgtgcccgatcatgagagcaataccgcgctcaaagccgacgcatagcctttactccgcaatccaaacgactgtcgctcgtattttttggatatctattttaaagagcgagcacagcgccgggcatgggcctgaaaggcctcgcggccgtgctcgtggtgggggccgcgagcgcgtggggcatcgcggcagtgcaccaggcgcagacggaggaacgcatggtgcgtgcgcaatataagatacatgtattgttgtcctgcagg

SEQ ID NO: 132 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgcctcccagctgcgcaagcccgccctggaccccctgcgcgccgtgatctccgccgaccagggctccatctcccccgtgaactcctgcacccccgccgaccgcctgcgcgccggccgcctgatggaggacggctactcctacaaggagaagttcatcgtgcgctcctacgaggtgggcatcaacaagaccgccaccgtggagaccatcgccaacctgctgcaggaggtggcctgcaaccacgtgcagaagtgcggcttctccaccgccggcttcgccaccaccctgaccatgcgcaagctgcacctgatctgggtgaccgcccgcatgcacatcgagatctacaagtaccccgcctggtccgacgtggtggagatcgagacctggtgccagtccgagggccgcatcggcacccgccgcgactggatcctgcgcgactccgccaccaacgaggtgatcggccgcgccacctccaagtgggtgatgatgaaccaggacacccgccgcctgcagcgcgtgaccgacgaggtgcgcgacgagtacctggtgttctgcccccgcgagccccgcctggccttccccgaggagaacaactcctccctgaagaagatccccaagctggaggaccccgcccagtactccatgctggagctgaagccccgccgcgccgacctggacatgaaccagcacgtgaacaacgtgacctacatcggctgggtgctggagtccatcccccaggagatcatcgacacccacgagctgcaggtgatcaccctggactaccgccgcgagtgccagcaggacgacatcgtggactccctgaccacctccgagatccccgacgaccccatctccaagttcaccggcaccaacggctccgccatgtcctccatccagggccacaacgagtcccagttcctgcacatgctgcgcctgtccgagaacggccaggagatcaaccgcggccgcacccagtggcgcaagaagtcctcccgcatggactacaaggaccacgacggcgactacaaggaccacgacatcgactacaaggacgacgacgacaagtga

SEQ ID NO: 133 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgcctcccagctgcgcaagcccgccctggaccccctgcgcgccgtgatctccgccgaccagggctccatctcccccgtgaactcctgcacccccgccgaccgcctgcgcgccggccgcctgatggaggacggctactcctacaaggagaagttcatcgtgcgctcctacgaggtgggcatcaacaagaccgccaccgtggagaccatcgccaacctgctgcaggaggtggcctgcaaccacgtgcagaagtgcggcttctccaccgacggcttcgccaccaccctgaccatgcgcaagctgcacctgatctgggtgaccgcccgcatgcacatcgagatctacaagtaccccgcctggtccgacgtggtggagatcgagacctggtgccagtccgagggccgcatcggcacccgccgcgactggatcctgcgcgactccgccaccaacgaggtgatcggccgcgccacctccaagtgggtgatgatgaaccaggacacccgccgcctgcagcgcgtgaccgccgaggtgcgcgacgagtacctggtgttctgcccccgcgagccccgcctggccttccccgaggagaacaactcctccctgaagaagatccccaagctggaggaccccgcccagtactccatgctggagctgaagccccgccgcgccgacctggacatgaaccagcacgtgaacaacgtgacctacatcggctgggtgctggagtccatcccccaggagatcatcgacacccacgagctgcaggtgatcaccctggactaccgccgcgagtgccagcaggacgacatcgtggactccctgaccacctccgagatccccgacgaccccatctccaagttcaccggcaccaacggctccgccatgtcctccatccagggccacaacgagtcccagttcctgcacatgctgcgcctgtccgagaacggccaggagatcaaccgcggccgcacccagtggcgcaagaagtcctcccgcatggactacaaggaccacgacggcgactacaaggaccacgacatcgactacaaggacgacgacgacaagtga

SEQ ID NO: 134 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgcctcccagctgcgcaagcccgccctggaccccctgcgcgccgtgatctccgccgaccagggctccatctcccccgtgaactcctgcacccccgccgaccgcctgcgcgccggccgcctgatggaggacggctactcctacaaggagaagttcatcgtgcgctcctacgaggtgggcatcaacaagaccgccaccgtggagaccatcgccaacctgctgcaggaggtggcctgcaaccacgtgcagaagtgcggcttctccaccgccggcttcgccaccaccctgaccatgcgcaagctgcacctgatctgggtgaccgcccgcatgcacatcgagatctacaagtaccccgcctggtccgacgtggtggagatcgagacctggtgccagtccgagggccgcatcggcacccgccgcgactggatcctgcgcgactccgccaccaacgaggtgatcggccgcgccacctccaagtgggtgatgatgaaccaggacacccgccgcctgcagcgcgtgaccgccgaggtgcgcgacgagtacctggtgttctgcccccgcgagccccgcctggccttccccgaggagaacaactcctccctgaagaagatccccaagctggaggaccccgcccagtactccatgctggagctgaagccccgccgcgccgacctggacatgaaccagcacgtgaacaacgtgacctacatcggctgggtgctggagtccatcccccaggagatcatcgacacccacgagctgcaggtgatcaccctggactaccgccgcgagtgccagcaggacgacatcgtggactccctgaccacctccgagatccccgacgaccccatctccaagttcaccggcaccaacggctccgccatgtcctccatccagggccacaacgagtcccagttcctgcacatgctgcgcctgtccgagaacggccaggagatcaaccgcggccgcacccagtggcgcaagaagtcctcccgcatggactacaaggaccacgacggcgactacaaggaccacgacatcgactacaaggacgacgacgacaagtga

SEQ ID NO: 135 - DNA, 5451 bp, Artificial Sequence
  Description of Artificial Sequence Synthetic polynucleotide
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgatcgagcaggacggcctccacgccggctcccccgccgcctgggtggagcgcctgttcggctacgactgggcccagcagaccatcggctgctccgacgccgccgtgttccgcctgtccgcccagggccgccccgtgctgttcgtgaagaccgacctgtccggcgccctgaacgagctgcaggacgaggccgcccgcctgtcctggctggccaccaccggcgtgccctgcgccgccgtgctggacgtggtgaccgaggccggccgcgactggctgctgctgggcgaggtgcccggccaggacctgctgtcctcccacctggcccccgccgagaaggtgtccatcatggccgacgccatgcgccgcctgcacaccctggaccccgccacctgccccttcgaccaccaggccaagcaccgcatcgagcgcgcccgcacccgcatggaggccggcctggtggaccaggacgacctggacgaggagcaccagggcctggcccccgccgagctgttcgcccgcctgaaggcccgcatgcccgacggcgaggacctggtggtgacccacggcgacgcctgcctgcccaacatcatggtggagaacggccgcttctccggcttcatcgactgcggccgcctgggcgtggccgaccgctaccaggacatcgccctggccacccgcgacatcgccgaggagctgggcggcgagtgggccgaccgcttcctggtgctgtacggcatcgccgcccccgactcccagcgcatcgccttctaccgcctgctggacgagttcttctgacaattgacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtgaatcgatgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttgagctccagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 136 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTGGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 137 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTGGFATTPTMRKLRLIWVTARMHIEIYKYPAWSDVVEIESWGQGEGKIGTRRDWILRDYATGQVIGRATSKWVMMNQDTRRLQKVDADVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 138 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTGGFVTTPTMRKLRLIWVTARMHIEIYKYPAWSDVVEIESWGQGEGKIGTRRDWILRDYATGQVIGRATSKWVMMNQDTRRLQKVDADVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 139 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVACNHAQSVGYSTGGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 140 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVTCNHAQSVGYSTGGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 141 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVVCNHAQSVGYSTGGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 142 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTAGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 143 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANFLQEVGCNHAQSVGYSTGGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 144 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANKLQEVGCNHAQSVGYSTGGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 145 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANSLQEVGCNHAQSVGYSTGGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 146 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTVGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 147 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTGGFSTTPTMRKLRLIWVTARMHIEIYKYPAWSDVVEIESWGQGEGKIGFRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 148 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTGGFSTTPTMRKLRLIWVTARMHIEIYKYPAWSDVVEIESWGQGEGKIGARRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 149 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTGGFSTTPTMRKLRLIWVTARMHIEIYKYPAWSDVVEIESWGQGEGKIGKRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 150 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTGGFSTTPTMRKLRLIWVTARMHIEIYKYPAWSDVVEIESWGQGEGKIGVRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 151 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttcgccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgcggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 152 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttcgtcaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgcggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 153 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtggcgtgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 154 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgacgtgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 155 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtggtgtgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 156 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccgccggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 157 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacttcctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 158 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacaagctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 159 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaactcgctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 160 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccgtcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 161 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcttccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 162 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcgcgcgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 163 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcaagcgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 164 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcgtgcgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 165 - protein, 391 aa, Artificial Sequence
  Description of Artificial Sequence Synthetic polypeptide
MATASTFSAFNARCGDLRRSAGSGPRRPARPLPVRGRASQLRKPALDPLRAVISADQGSISPVNSCTPADRLRAGRLMEDGYSYKEKFIVRSYEVGINKTATVETIANLLQEVACNHVQKCGFSTDGFATTLTMRKLHLIWVTARMHIEIYKYPAWSDVVEIETWCQSEGRIGTRRDWILRDSATNEVIGRATSKWVMMNQDTRRLQRVTDEVRDEYLVFCPREPRLAFPEENNSSLKKIPKLEDPAQYSMLELKPRRADLDMNQHVNNVTYIGWVLESIPQEIIDTHELQVITLDYRRECQQDDIVDSLTTSEIPDDPISKFTGTNGSAMSSIQGHNESQFLHMLRLSENGQEINRGRTQWRKKSSRMDYKDHDGDYKDHDIDYKDDDDK

SEQ ID NO: 166 - protein, 391 aa, Artificial Sequence
  Description of Artificial Sequence Synthetic polypeptide
MATASTFSAFNARCGDLRRSAGSGPRRPARPLPVRGRASQLRKPALDPLRAVISADQGSISPVNSCTPADRLRAGRLMEDGYSYKEKFIVRSYEVGINKTATVETIANLLQEVACNHVQKCGFSTAGFATTLTMRKLHLIWVTARMHIEIYKYPAWSDVVEIETWCQSEGRIGTRRDWILRDSATNEVIGRATSKWVMMNQDTRRLQRVTDEVRDEYLVFCPREPRLAFPEENNSSLKKIPKLEDPAQYSMLELKPRRADLDMNQHVNNVTYIGWVLESIPQEIIDTHELQVITLDYRRECQQDDIVDSLTTSEIPDDPISKFTGTNGSAMSSIQGHNESQFLHMLRLSENGQEINRGRTQWRKKSSRMDYKDHDGDYKDHDIDYKDDDDK

SEQ ID NO: 167 - protein, 391 aa, Artificial Sequence
  Description of Artificial Sequence Synthetic polypeptide
MATASTFSAFNARCGDLRRSAGSGPRRPARPLPVRGRASQLRKPALDPLRAVISADQGSISPVNSCTPADRLRAGRLMEDGYSYKEKFIVRSYEVGINKTATVETIANLLQEVACNHVQKCGFSTDGFATTLTMRKLHLIWVTARMHIEIYKYPAWSDVVEIETWCQSEGRIGTRRDWILRDSATNEVIGRATSKWVMMNQDTRRLQRVTAEVRDEYLVFCPREPRLAFPEENNSSLKKIPKLEDPAQYSMLELKPRRADLDMNQHVNNVTYIGWVLESIPQEIIDTHELQVITLDYRRECQQDDIVDSLTTSEIPDDPISKFTGTNGSAMSSIQGHNESQFLHMLRLSENGQEINRGRTQWRKKSSRMDYKDHDGDYKDHDIDYKDDDDK

SEQ ID NO: 168 - protein, 391 aa, Artificial Sequence
  Description of Artificial Sequence Synthetic polypeptide
MATASTFSAFNARCGDLRRSAGSGPRRPARPLPVRGRASQLRKPALDPLRAVISADQGSISPVNSCTPADRLRAGRLMEDGYSYKEKFIVRSYEVGINKTATVETIANLLQEVACNHVQKCGFSTAGFATTLTMRKLHLIWVTARMHIEIYKYPAWSDVVEIETWCQSEGRIGTRRDWILRDSATNEVIGRATSKWVMMNQDTRRLQRVTAEVRDEYLVFCPREPRLAFPEENNSSLKKIPKLEDPAQYSMLELKPRRADLDMNQHVNNVTYIGWVLESIPQEIIDTHELQVITLDYRRECQQDDIVDSLTTSEIPDDPISKFTGTNGSAMSSIQGHNESQFLHMLRLSENGQEINRGRTQWRKKSSRMDYKDHDGDYKDHDIDYKDDDDK

SEQ ID NO: 169 - protein, 385 aa, Artificial Sequence; identical to SEQ ID NO: 23
  Description of Artificial Sequence Synthetic polypeptide

SEQ ID NO: 170 - protein, 384 aa, Artificial Sequence; identical to SEQ ID NO: 24
  Description of Artificial Sequence Synthetic polypeptide

SEQ ID NO: 171 - protein, 391 aa, Artificial Sequence; identical to SEQ ID NO: 28
  Description of Artificial Sequence Synthetic polypeptide

SEQ ID NO: 172 - protein, 391 aa, Artificial Sequence; identical to SEQ ID NO: 29
  Description of Artificial Sequence Synthetic polypeptide

SEQ ID NO: 173 - protein, 369 aa, Artificial Sequence; identical to SEQ ID NO: 34
  Description of Artificial Sequence Synthetic polypeptide

SEQ ID NO: 174 - protein, 387 aa, Artificial Sequence; identical to SEQ ID NO: 76
  Description of Artificial Sequence Synthetic polypeptide

SEQ ID NO: 175 - protein, 387 aa, Artificial Sequence; identical to SEQ ID NO: 75
  Description of Artificial Sequence Synthetic polypeptide

SEQ ID NO: 176 - protein, 387 aa, Artificial Sequence
  Description of Artificial Sequence Synthetic polypeptide
MAIAAAAVIVPLGLLFFVSGLIVNLVQAVCFVLIRPLSKNTYRRINRVVAELLWLELVWLIDWWAGVKIKVFTDHETLSLMGKEHALVICNHKSDIDWLVGWVLAQRSGCLGSTLAVMKKSSKFLPVIGWSMWFSEYLPESDDAVAQWCRDIFVEKDALLDKHNAEDTFSGQELQDTGRPIKSLLVVISWAVLVIFGAVKFLQWSSLLSSWKGLAFSGVGLGIITLLMHILILFSQSERSTPAKVAPAKPKKDGESSKTEIEKENVPGALLGQGREHPEVRPEPPEGLPPALLAGPVRGGHPLHPRQAAGRPAVRHLLRPARAPQRADPPHQGLRVLRVPHALLRARHLRRDRGHPQDLPPPHHAAHVQGPVLRAARAPEAPPDEGP

SEQ ID NO: 177 - protein, 384 aa, Artificial Sequence
  Description of Artificial Sequence Synthetic polypeptide
MAIAAAAVIFLFGLIFFASGLIINLFQALCFVLIRPLSKNAYRRINRVFAELLLSELLCLFDWWAGAKLKLFTDPETFRLMGKEHALVIINHMTELDWMVGWVMGQHFGCLGSIISVAKKSTKFLPVLGWSMWFSEYLYLERSWAKDKSTLKSHIERLIDYPLPFWLVIFVEGTRFTRTKLLAAQQYAVSSGLPVPRNVLIPRTKGFVSCVSHMRSFVPAVYDVTVAFPKTSPPPTLLNLFEGQSIMLHVHIKRHAMKDLPESDDAVAEWCRDKFVEKDALLDKHNAEDTFSGQEVCHSGSRQLKSLLVVISWVVVTTFGALKFLQWSSWKGKAFSAIGLGIVTLLMHVLILSSQAERSNPAEVAQAKLKTGLSISKKVTDKEN

SEQ ID NO: 178 - protein, 380 aa, Artificial Sequence
  Description of Artificial Sequence Synthetic polypeptide
MAIPAAVAVIPIGLLFIISGLIVNLIQAVVYVLIRPLSKNLHRKINKPIAELLWLELIWLVDWWAGIKVEVYADSQTLELMGKEHALLICNHRSDIDWLVGWVLAQRARCLGSALAIMKKSAKFLPVIGWSMWFSDYIFLDRTWAKDEKTLKSGFERLADFPMPFWLALFVEGTRFTKAKLLAAQEYAASRGLPVPQNVLIPRTKGFVTAVTHMRSYVPAIYDCTVDISKAHPAPSILRLIRGQSSVVKVQITRHSMQELPETADGISQWCMDLFVTKDGFLEKYHSKDIFGSLPVQNIGRPVKSLIVVLCWYCLMAFGLFKFFMWSSLLSSWEGILSLGLILLAVAIVMQILIQSTESERSTPVKSIQKDPSKETLLQN

SEQ ID NO: 179 - protein, 382 aa, Artificial Sequence; identical to SEQ ID NO: 85
  Description of Artificial Sequence Synthetic polypeptide

SEQ ID NO: 180 - protein, 380 aa, Artificial Sequence
  Description of Artificial Sequence Synthetic polypeptide
MAIPAAVAVIPIGLLFIISGLIVNLIQAVVYVLIRPLSKNLYRKINKPIAELLWLELIWLVDWWAGIKVEVYADSETLESMGKEHALLICNHRSDIDWLVGWVLAQRARCLGSALAIMKKSAKFLPVIGWSMWFSDYIFLDRTWEKDEKTLKSGFERLADFPMPFWLALFVEGTRFTKAKLLAAQEFAASRGLPVPQNVLIPRTKGFVTAVTHMRSYVPAIYDCTVDISKAHPAPSILRLIRGQSSVVKVQITRHSMQELPETPDGISQWCMDLFVTKDAFLEKYHSKDIFGSLPVHDIGRPVKSLIVVLCWYSLMAFGFYKFFMWSSLLSSWEGILSLGLVLIVIAIVMQILIQSSESERSTPVKSVQKDPSKETLLQN

SEQ ID NO: 181 - protein, 374 aa, Artificial Sequence
  Description of Artificial Sequence Synthetic polypeptide
MATGGSLKPSSSDLDLDHPNIEDYLPSGSSINEPAGKLRLRDLLDISPTLTEAAGAIVDDSFTRCFKSIPREPWNWNLYLFPLWCIGVLIRYFILFPGRVIVLTMGWITVISSFIAVRVLLKGHDALQIKLERLIVQLLCSSFVASWTGVVKYHGPRPSIRPKQVYVANHTSMIDFFILDQMTVFSVIMQKHPGWVGLLQSTLLESVGCIWFDRAEAKDRGIVAKKLWDHVHGEGNNPLLIFPEGTCVNNNYSVMFKKGAFELGCTVCPVAIKYNKIFVDAFWNSKKQSFTRHLLQLMTSWAVVCDVWYLEPQTLKPGETPIEFAERVRDIISARAGLKKVPWDGYLKYSRPSPKHRERKQQTFAESVLQRLEE

SEQ ID NO: 182 - protein, 375 aa, Artificial Sequence
  Description of Artificial Sequence Synthetic polypeptide
MATAGSLKPSRSELDFDRPNIEDYLPSGSSIIEPAGKLRLRDLLDISPTLTEAAGAIVDDSFTRCFKSNPPEPWNWNIYLFPLWCFGVLIRYLILFPARVIVLTIGWIIFLSSFIPVHLLLKGHDALRIKLERLLVELICSFFVASWTGVVKYHGPRPSIRPKQVYVANHTSMIDFFILDQMTVFSVIMQKHPGWVGLLQSTLLESVGCIWFDRAEAKDRGIVAKKLWDHVHGEGNNPLLIFPEGTCVNNNYSVMFKKGAFELGCTVCPVAIKYNKIFVDAFWNSKKQSFTRHLLQLMTSWAVVCDVWYLEPQTLKPGETPIEFAERVRDIISVRAGLKKVPWDGYLKYSRPSPKHTERKQQNFAESVLQRLEKK

SEQ ID NO: 183 - protein, 375 aa, Artificial Sequence
  Description of Artificial Sequence Synthetic polypeptide
MATGGRLKPSSSELDLDRANTEDYLPSGSSINEPVGKLRLRDLLDISPTLTEAAGAIVDDSFTRCFKSIPPEPWNWNIYLFPLWCFGVLIRYFILFPARVIVLTIGWITVISSFTAVRFLLKGHNALQIKLERLIVQLLCSSFVASWTGVVKYHGPRPSIRPKQVYVANHTSMIDFLILDQMTVFSVIMQKHPGWVGLLQSTLLESVGCIWFNRAEAKDREIVAKKLWDHVHGEGNNPLLIFPEGTCVNNHYSVMFKKGAFELGCTVCPVAIKYNKIFVDAFWNSRKQSFTMHLLQLMTSWAVVCDVWYLEPQTLKPGETAIEFAERVRDIISVRAGLKKVPWDGYLKYSRPSPKHRESKQQSFAESVLRRLEEK

SEQ ID NO: 184 - protein, 375 aa, Artificial Sequence
  Description of Artificial Sequence Synthetic polypeptide
MATGGRLKPSSSELDLDRANTEDYLPSGSSINEPVGKLRLRDLLDISPTLTEAAGAIVDDSFTRCFKSIPPEPWNWNIYLFPLWCFGVLIRYFILFPARVIVLTIGWITVISSFTAVRFLLKGHNALQIKLERLIVQLLCSSFVASWTGVVKYHGPRPSIRPKQVYVANHTSMIDFLILDQMTVFSVIMQKHPGWVGLLQSTLLESVGCIWFNRAEAKDREIVAKKLWDHVHGEGNNPLLIFPEGTCVNNHYSVMFKKGAFELGCTVCPVAIKYNKIFVDAFWNSKKHSFTRHLLQLMTSWAVVCDVWYLEPQTLKPGETPIEFAERVRDIISVRADLKKVPWDGYLKYSRPSPKHRERKQQKFAESVLRRLEEK

SEQ ID NO: 185 - protein, 377 aa, Artificial Sequence
  Description of Artificial Sequence Synthetic polypeptide
MATAGRLKPSSSELELDLDRPNIEDYLPSGSSINEPAGKLRLRDLLDISPMLTEAAGAIVDDSFTRCFKSIPPEPWNWNIYLFPLWCFGVLIRYLILFPARVIVLTVGWITVISSFITVRFLLKGHDSLRIKLERLIVQLFCSSFVASWTGVVKYHGPRPSIRPQQVYVANHTSMIDFIILNQMTVFSAIMQKHPGWVGLIQSTILESVGCIWFNRAEAKDREIVAKKLLDHVHGEGNNPLLIFPEGTCVNNHYSVMFKKGAFELGCTVCPVAIKYNKIFVDAFWNSKKQSFTMHLLQLMTSWAVVCDVWYLEPQTLKPGETPIEFAERVRDIISVRAGLKKVPWDGYLKYSRPSPKHRERKQQSFAESVLRRLEKR

SEQ ID NO: 186 - protein, 385 aa, Artificial Sequence
  Description of Artificial Sequence Synthetic polypeptide
MATAGRLKPSSSELELDLDRPNIEDYLPSGSSINEPAGKLRLRDLLDISPMLTEAAGAIVDDSFTRCFKSIPPEPWNWNIYLFPLWCFGVLIRYLILFPARVIVLTVGWITVISSFITVRFLLKGHDSLRIKLERLIVQLFCSSFVASWTGVVKYHGPRPSIRPQQVYVANHTSMIDFIILNQMTVFSAIMQKHPGWVGLIQSTILESVGCIWFNRAEAKDREIVAKKLLDHVHGEGNNPLLIFPEGTCVNNHYSVMFKKGAFELGCTVCPVAIKYNKIFVDAFWNSKKLSFTMHLLQLMTSWAVVCDVWYLEPQTLKPGETPIEFAERVRDIISVRAGLKKVPWDGYLKYSRPSPKHRERKQQTFAESVLRRLEEKGNVVPTVN

SEQ ID NO: 187 - protein, 524 aa, Artificial Sequence
  Description of Artificial Sequence Synthetic polypeptide
MAIADGGIIGAAGSISALTADTDPPSLRRRNVPAGQASAVSAFSTESMAKHLCDPSREPSPSPKSSDDGKDPDIGSVDSLNEKPSSPAAGKGRLQHDLRFTYRASSPAHRKVKESPLSSSNIFKQSHAGLFNLCVVVLVAVNSRLIIENLMKYGLLIKTGFWFSSRSLRDWPLFMCCLSLPIFPLAAFLVEKLAQKNRLQEPTVVCCHVLITSVSILYPVLVILRCDSAVLSGVALMLFACIVWLKLVSYAHSNYDMRYVAKSLDKGEPVVDSVIADHPYRVDYKDLVYFMVAPTLCYQLSYPLTPCVRKSWIARQVMKLVLFTGVMGFIVEQYINPIVQNSKHPLKGDLLYAIERVLKLSVPNLYVWLCMFYCFFHLWLNILAELICFGDREFYKDWWNAKTVEEYWRMWNMPVHKWMVRHIYFPCLRNGIPRGVAVLIAFLVSAVFHELCIAVPCHVFKLWAFIGIMFQVPLVLVSNCLQKKFQSSMAGNMFFWFIFCIFGQPMCVLLYYHDLMNRKGSRID

SEQ ID NO: 188 - protein, 528 aa, Artificial Sequence
  Description of Artificial Sequence Synthetic polypeptide
MAIADGGSAGAAGSISGSDPSPSTAPSLRRRNASAGQAFSTESMARDLCDPSREPSLSPKSSDDGKDPADDIGAADSVDSGGVKDEKPSSQAAAKARLEHDLRFTYRASSPAHRKVKESPLSSSNIFKQSHAGLFNLCVVVLVAVNSRLIIENLMKYGLLIKTGFWFSSRSLRDWPLFMCCLSLPIFPLAAFLVEKLAQKNRLQEPTVVCCHVIITSVSILYPVLVILRCDSAVLSGVALMLFACIVWLKLVSYAHANYDMRSVAKSLDKGETVADSVIVDHPYRVDYKDLVYFMVAPTLCYQLSYPLTPYVRKSWVARQVMKLVLFTGVMGFIVEQYINPIVQNSKHPLKGDLLYAIERVLKLSVPNLYVWLCMFYCFFHLWLNILAELTCFGDREFYKDWWNAKTVEEYWRMWNMPVHKWMVRHIYFPCLRNGIPRGVAVLIAFLVSAVFHELCIAVPCHVFKLWAFIGIMFQVPLVLVSNCLQKKFQSSMAGNMFFWFIFCIFGQPMCVLLYYHDLMNRKGSRID

SEQ ID NO: 189 - protein, 463 aa, Artificial Sequence
  Description of Artificial Sequence Synthetic polypeptide
MGLVSVAAAIGVSVPVARFLLCFLATIPVSFLWRLVPGRLPKHLYSAASGAILSYLSFGASSNLHFIVPMTLGYLSMLFFRPFSGLLTFFLGFGYLIGCHVYYMSGDAWKEGGIDATGALMVLTLKVISCSMNYNDGLLKEEGLRESQKKNRLTKMPSLIEYFGYCLCCGSHFAGPVYEMKDYLEWTEGKGIWSRSQKEPKPSPFGGALRAIIQAAVCMAMYLYLVPHHPLTRFTEPVYYEWGFFRRLSYQYMAALTARWKYYFIWSISEASLIISGLGFSGWTESSPPKPRWDRAKNVDIIGVEFAKSSVQLPLVWNIQVSIWLRHYVYDRLVQNGKRPGFFQLLATQTVSAVWHGLYPGYIIFFVQSALMIAGSRVIYRWQQAVPPKMGLVKNIFVFFNFAYTLLVLNYSAVGFMVLSMHETLASYGSVYYIGTILPITLILLSYVIKPGKPARSKAHKEQ

SEQ ID NO: 190 - protein, 463 aa, Artificial Sequence
  Description of Artificial Sequence Synthetic polypeptide
MELGSVAAAIGVSVPVARFLLCFLATIPVSFLWRLVPGRLPKHLYSAASGAILSYLSFGPSSNLHFIVPMTLGYLSMLFFRPFSGLLTFFLGFGYLIGCHVYYMSGDAWKEGGIDATGALMVLTLKVISCSINYNDGLLKEEGLRESQKKNRLTKMPSLIEYIGYCLCCGSHFAGPVYEMKDYLEWTEGKGVWSHSEKEPKPSPFGGALRAIIQAAVCMAMYMYLVPHHPLSRFTEPVYYEWGFFRRLSYQYMAGLTARWKYYFIWSISEASLIISGLGFSGWTESSPPKPRWDRAKNVDIIGVEFAKSSVQLPLVWNIQVSTWLRHYVYDRLVQNGKRPGFFQLLATQTVSAIWHGLYPGYIIFFVQSALMIAGSRVIYRWQQAVPPKMGLVKNIFVFFNFAYTLLVLNYSAVGFMVLSMHETLASYGSVYYIGTILPITLILLSYVIKPGKPARSKAHKEQ

SEQ ID NO: 191 - protein, 465 aa, Artificial Sequence
  Description of Artificial Sequence Synthetic polypeptide
MELEIGSVAAAIGVSVPVARFLLCFLATIPVSFLCRLLPARLPKHLYSAASGAILSYLSFGPSSNLHFIVPMSLGYLSMLFFRPFSGLLTFFLGFGYLIGCHVYYMSGDAWKEGGIDATGALMVLTLKVISCSINYNDGLLKEEGLRESQKKNRLTKMPSLIEYFGYCLCCGSHFAGPVYEMKDYLEWTEGKGIWSRSEKDPKPSPFGGALRAIIQAAVCMAMHMYLVPHHPLTRFTEPVYYEWGFFRRLSYQYMAAQTARWKYYFIWSISEASLIISGLGFSGWTESSPPKPRWDKAKNVDIIGVEFAKSSVQLPLVWNIQVSTWLRHYVYDRLVQNGKRPGFFQLLATQTVSAVWHGLYPGYIIFFVQSALMIAGSRVIYRWQQAVPQKMGLVKNIFVFFNFAYTLLVLNYSAVGFMVLSMHETLASYGSVYYIGTILPITLILLSYVIKPGKPTRSKVHKEQ

SEQ ID NO: 192 - protein, 465 aa, Artificial Sequence
  Description of Artificial Sequence Synthetic polypeptide
MELEMEPLAAAIGVSVAVFRFLVCFIATIPVSFICRLVPGGLPRHLFSAASGAVLSYLSFGFSSNLHFLVPMTLGYLSMILFRRFCGILTFFLGFGYLIGCHVYYMSGDAWKEGGIDATGALMVLTLKVISCSINYNDGLLKEEGLRESQKKNRLIRLPSLIEYFGYCLCCGSHFAGPVYEMKDYLDWTEGKGIWSHSEKGPKPSPLRAALRAIIQAGFCMAMYLYLVPHYPLTRFTDPVYYEWGILRRLSYQYMASFTARWKYYFIWSISEASLIISGLGFSGWTESSPPKPRWDRAKNVDILGVELAKSSVQIPLVWNIQVSTWLRHYVYDRLVQNGKRPGFLQLLATQTVSAIWHGVYPGYLIFFVQSALMIAGSRAIYRWQQAVPPKMSLVKNTLVFFNFAYTLLVLNYSAVGFMVLSMHETLASYGSVYYVGTILPVTLILLGYVIKPGKSPRSKASKEQ

SEQ ID NO: 193 - protein, 245 aa, Artificial Sequence
  Description of Artificial Sequence Synthetic polypeptide
MNFDFLSNIPWFGAKASDNAGSSFGSATIVIQQPPPVSRGFDIRHWGWPWSVLSVLPWGKPGCDELRAPPTTINRRLKRNATSMHSSAVRGNAEAARVRFRPYVSKVPWHTGFRGLLSQLFPRYGHYCGPNWSSGKNGGSPVWDQRPIDWLDYCCYCHDIGYDTHDQAKLLEADLAFLECLERPSYPTKGDAHVAHMYKTMCVTGLRNVLIPYRTQLLRLNSRQPLIDFGWLSNAAWKGWNAQKS

SEQ ID NO: 194 - protein, 236 aa, Artificial Sequence
  Description of Artificial Sequence Synthetic polypeptide
MNLDFLSKIPWFEAKASENPGLNLGSTTIVIKQPRQGFDIRHWGWPWSVLTWGNRVTDEVHAPPTTINRRLKRNATGPAVQGDTEAARLRFRPYVSKVPWHTGFRGLLSQLFPRYGHYCGPNWSSGKNGGSPVWDQRPIDWLDYCCYCHDIGYDTHDQAKLLEADLAFLECLERPSYPTTGDAHVAHMYKTMCVTGLRNVLIPYRTQLLRLNFRQPLIDFGWLSNAAWKGWSAQKT

SEQ ID NO: 195 - protein, 158 aa, Artificial Sequence
  Description of Artificial Sequence Synthetic polypeptide
MVHLPHTLKLGLVIAISISGLCFSSTPARALNVGIQAAGVTVSVGKGCSRKCESDFCKVPPFLRYGKYCGLMYSGCPGEKPCDGLDACCMKHDACVQAKNNDYLSQECSQNLLNCMASFRMSGGKQFKGSTCQVDEVVDVLTVVMEAALLAGRYLHKP

SEQ ID NO: 196 - protein, 158 aa, Artificial Sequence
  Description of Artificial Sequence Synthetic polypeptide
MVHLPHTLKLGLVIAISISGLCLSSTPARALNVGIQAAGVTVSVGKGCSRKCESDFCKVPPFLRYGKYCGLMYSGCPGEKPCDGLDACCMKHDACVQAKNDDYLSQECSQNLLNCMASFRMSGGKQFKGSTCQVDEVVDVLTVVMEAALLAGRYLHKP

SEQ ID NO: 197 - DNA, 6405 bp, Artificial Sequence
  Description of Artificial Sequence Synthetic polynucleotide
agcggaagagcgcccaatgtttaaacccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattgacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcgcctgctcaagcgggcgctcaacatgcagagcgtcagcgagacgggctgtggcgatcgcgagacggacgaggccgcctctgccctgtttgaactgagcgtcagcgctggctaaggggagggagactcatccccaggctcgcgccagggctctgatcccgtctcgggcggtgatcggcgcgcatgactacgacccaacgacgtacgagactgatgtcggtcccgacgaggagcgccgcgaggcactcccgggccaccgaccatgtttacaccgaccgaaagcactcgctcgtatccattccgtgcgcccgcacatgcatcatcttttggtaccgacttcggtcttgttttacccctacgacctgccttccaaggtgtgagcaactcgcccggacatgaccgagggtgatcatccggatccccaggccccagcagcccctgccagaatggctcgcgctttccagcctgcaggcccgtctcccaggtcgacgcaacctacatgaccaccccaatctgtcccagaccccaaacaccctccttccctgcttctctgtgatcgctgatcagcaacaactagtaacaatggccaccgcctccaccttctccgccttcaacgcccgctgcggcgacctgcgccgctccgccggctccggcccccgccgccccgcccgccccctgcccgtgcgcgccgccatcaacgactccgcccaccccaaggccaacggctccgccgtgagcctgaagagcggcagcctgaacacccaggaggacacctcctccagcccccccccccgcaccttcctgcaccagctgcccgactggagccgcctgctgaccgccatcaccaccgtgttcgtgaagtccaagcgccccgacatgcacgaccgcaagtccaagcgccccgacatgctggtggacagcttcggcctggagtccaccgtgcaggacggcctggtgttccgccagtccttctccatccgctcctacgagatcggcaccgaccgcaccgccagcatcgagaccctgatgaaccacctgcaggagacctccctgaaccactgcaagagcaccggcatcctgctggacggcttcggccgcaccctggagatgtgcaagcgcgacctgatctgggtggtgatcaagatgcagatcaaggtgaaccgctaccccgcctggggcgacaccgtggagatcaacacccgcttcagccgcctgggcaagatcggcatgggccgcgactggctgatctccgactgcaacaccggcgagatcctggtgcgcgccaccagcgcctacgccatgatgaaccagaagacccgccgcctgtccaagctgccctacgaggtgcaccaggagatcgtgcccctgttcgtggacagccccgtgatcgaggactccgacctgaaggtgcacaagttcaaggtgaagaccggcgacagcatccagaagggcctgacccccggctggaacgacctggacgtgaaccagcacgtgtccaacgtgaagtacatcggctggatcctggagagcatgcccaccgaggtgctggagacccaggagctgtgctccctggccctggagtaccgccgcgagtgcggccgcgactccgtgctggagagcgtgaccgccatggaccccagcaaggtgggcgtgcgctcccagtaccagcacctgctgcgcctggaggacggcaccgccatcgtgaacggcgccaccgagtggcgccccaagaacgccggcgccaacggcgccatctccaccggcaagaccagcaacggcaactccgtgtccatggactacaaggaccacgacggcgactacaaggaccacgacatcgactacaaggacgacgacgacaagtgactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatttacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttgagctccagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgactgaagagcgttt

SEQ ID NO: 198 - DNA, 6922 bp, Artificial Sequence
  Description of Artificial Sequence Synthetic polynucleotide
agcggaagagcgcccaatgtttaaacccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattgacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcgaattcggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccactagtatggccaccgcctccaccttctccgccttcaacgcccgctgcggcgacctgcgccgctccgccggctccggcccccgccgccccgcccgccccctgcccgtgcgcgccgccatcaactcccgcgcccaccccaaggccaacggctccgccgtgtccctgaagtccggctccctgaacacccaggaggacacctcctcctccccccccccccgcaccttcctgcaccagctgcccgactggtcccgcctgctgaccgccatcaccaccgtgttcgtgaagtccaagcgccccgacatgcacgaccgcaagtccaagcgccccgacatgctgatggactccttcggcctggagtccatcgtgcaggagggcctggagttccgccagtccttctccatccgctcctacgagatcggcaccgaccgcaccgcctccatcgagaccctgatgaactacctgcaggagacctccctgaaccactgcaagtccaccggcatcctgctggacggcttcggccgcacccccgagatgtgcaagcgcgacctgatctgggtggtgaccaagatgaagatcaaggtgaaccgctaccccgcctggggcgacaccgtggagatcaacacctggttctcccgcctgggcaagatcggcaagggccgcgactggctgatctccgactgcaacaccggcgagatcctgatccgcgccacctccgcctacgccaccatgaaccagaagacccgccgcctgtccaagctgccctacgaggtgcaccaggagatcgcccccctgttcgtggactccccccccgtgatcgaggacaacgacctgaagctgcacaagttcgaggtgaagaccggcgactccatccacaagggcctgacccccggctggaacgacctggacgtgaaccagcacgtgtccaacgtgaagtacatcggctggatcctggagtccatgcccaccgaggtgctggagacccaggagctgtgctccctggccctggagtaccgccgcgagtgcggccgcgactccgtgctggagtccgtgaccgccatggaccccaccaaggtgggcggccgctcccagtaccagcacctgctgcgcctggaggacggcaccgacatcgtgaagtgccgcaccgagtggcgccccaagaaccccggcgccaacggcgccatctccaccggcaagacctccaacggcaactccgtgtccatggactacaaggaccacgacggcgactacaaggaccacgacatcgactacaaggacgacgacgacaagtgattaattaactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttgagctccagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgactgaagagcgtttaaaccgcct